(19) United States
(12) Patent Application Publication
(10) Pub. No.: US 2026/0173646 A1
(43) Pub. Date: Jun. 18, 2026

(54) COMPOUND AND ORGANIC LIGHT-EMITTING DEVICE COMPRISING SAME

(71) Applicant: LT MATERIALS CO.,LTD., Yongin-si (KR)

(72) Inventors: Hyun-Sun PARK, Yongin-si (KR); Won-Jang JEONG, Yongin-si (KR); Dong-Jun KIM, Yongin-si (KR); Dae-Hyuk CHOI, Yongin-si (KR)

(21) Appl. No.: 19/422,407

(22) Filed: Dec. 16, 2025

(30) Foreign Application Priority Data
Dec. 18, 2024 (KR) 10-2024-0189621
Nov. 24, 2025 (KR) 10-2025-0179311

(51) Int. Cl. H10K 50/15; C07B 59/00; C07C 211/54; C09K 11/06; H10K 50/18; H10K 85/60

(52) U.S. Cl. CPC H10K 50/15; C07B 59/004; C07C 211/54; C09K 11/06; H10K 50/181; H10K 85/622; H10K 85/626; H10K 85/633; H10K 85/636; H10K 85/654; H10K 85/6572; H10K 85/6574; H10K 85/6576; C07B 2200/05; C09K 2211/1011

(57) ABSTRACT

Provided are a compound and an organic light-emitting device containing the same. The compound may be used as an organic layer material of an organic light-emitting device. That is to say, it may serve as a hole transport material, an electron-blocking material, etc. in an organic light-emitting device. Specifically, when the compound is used in an organic layer of an organic light-emitting device, remarkably excellent effect may be achieved in terms of device operation, efficiency and lifetime.

### CROSS-REFERENCE TO RELATED APPLICATION

This application claims the priority of Korean Patent Application No. 10-2024-0189621, filed on Dec. 18, 2024, and Korean Patent Application No. 10-2025-0179311, filed on Nov. 24, 2025, and all the benefits accruing therefrom, the contents of which in their entirety are herein incorporated by reference.

### BACKGROUND

### 1. Field

The present disclosure relates to a compound and an organic light-emitting device containing the same.

### 2. Description of the Related Art

An electroluminescent device is a light-emitting display device which has the advantages of a wide viewing angle, excellent contrast, and a fast response speed.

An organic light-emitting device has a structure in which an organic thin film is placed between two electrodes. When a voltage is applied to the organic light-emitting device, electrons and holes injected from the two electrodes recombine in the organic thin film and emit light as they disappear. The organic thin film may consist of a single layer or multiple layers, as needed.

The material of the organic thin film can have light-emitting function, if necessary. For example, as the organic thin film material, a compound that can form a light-emitting layer on its own may be used, or a compound that can serve as a host or a dopant of a host-dopant light-emitting layer may be used. In addition, a compound that can perform the function of hole injection, hole transport, electron blocking, hole blocking, electron transport, electron injection, etc. may be used as the organic thin film material.

The development of organic thin film materials is continuously required to improve the performance, efficiency and lifetime of the organic light-emitting device.

### REFERENCES OF THE RELATED ART

### Patent Documents

(Patent document 1) U.S. Pat. No. 4,356,429.

### SUMMARY

The present disclosure is directed to providing a compound and an organic light-emitting device containing the same.

In an exemplary embodiment of the present application, there is provided a compound represented by Chemical Formula 1.

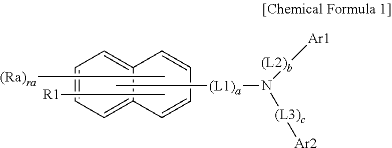

In Chemical Formula 1,
R1 is a substituted or unsubstituted C₁ to C₆₀ branched alkyl group,
L1 is a substituted or unsubstituted C₆ to C₆₀ arylene group,
a is an integer from 1 to 3, wherein if a is 2 or greater, L1's are identical or different,
L2 and L3, which are identical or different, are independently a direct bond; or a substituted or unsubstituted C₆ to C₆₀ arylene group,
b and c are respectively an integer from 0 to 3, wherein if b is 2 or greater, L2's are identical or different, and if c is 2 or greater, L3's are identical or different,
Ra is hydrogen; deuterium; a halogen group; a cyano group; a substituted or unsubstituted C₁ to C₆₀ alkyl group; a substituted or unsubstituted C₆ to C₆₀ aryl group; or a substituted or unsubstituted C₂ to C₆₀ heteroaryl group,
ra is an integer from 0 to 6, wherein if ra is 2 or greater, Ra's are identical or different,
with the proviso that L1 and R1 are not bonded at para positions,
Ar1 and Ar2, which are identical or different, are independently a substituted or unsubstituted C₄ to C₆₀ monocyclic or polycyclic aliphatic ring group; a substituted or unsubstituted C₆ to C₆₀ aryl group consisting of one or more benzene ring; a substituted or unsubstituted C₂ to C₆₀ tricyclic heteroaryl group; or Structural Formula A,

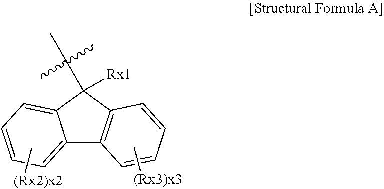

in Structural Formula A,

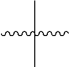

is a moiety connected to Chemical Formula 1,
Rx1 is a substituted or unsubstituted C₁ to C₆₀ alkyl group; a substituted or unsubstituted C₄ to C₆₀ monocyclic or polycyclic aliphatic ring group; or a substituted or unsubstituted C₆ to Coo aryl group,
Rx2 and Rx3 are hydrogen; deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group, or two or more adjacent substituents are bonded to each other to form a substituted or unsubstituted ring, and
x2 and x3 are integers from 0 to 4, wherein if x2 and x3 are respectively 2 or greater, the substituents in the parentheses are identical or different.

In another exemplary embodiment of the present application, there is provided an organic light-emitting device including a first electrode; a second electrode facing the first electrode; and one or more organic layer provided between the first electrode and the second electrode, wherein one or more layer of the organic layer contains the compound represented by Chemical Formula 1 described above.

The compound described in the present specification may be used as an organic layer material of an organic light-emitting device. That is to say, it may serve as a hole transport material, an electron-blocking material, etc. in an organic light-emitting device.

Specifically, when the compound is used in an organic layer of an organic light-emitting device, remarkably excellent effect may be achieved in terms of device operation, efficiency and lifetime.

### BRIEF DESCRIPTION OF THE DRAWINGS

FIGS. 1 to 4 schematically show the stacking structures of organic light-emitting devices according to exemplary embodiments of the present application.

### DETAILED DESCRIPTION

Hereinafter, the present specification is described in more detail.

In the present specification, when a part is said to “include” a certain component, this does not mean that other components are excluded, but rather that other components may be further included, unless the context specially states otherwise.

In the present specification,

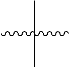

in chemical formulas refers to the site of bonding.

In the present specification, n in Cn means the number of carbon atoms. For example, C₆ to C₆₀ means 6 to 60 carbon atoms.

In the present specification, the term “substitution” means that a hydrogen atom bonded to a carbon atom of a compound is replaced with another substituent, and the position of substitution is not limited as long as it is the position where substitution is possible. When there are two or more substitutions, the two or more substituents may be identical to or different from each other.

In the present specification, “substituted or unsubstituted” means being substituted or unsubstituted with one or more substituent selected from a group consisting of deuterium; a halogen group; —CN; a C₁ to C₆₀ alkyl group; a C₂ to C₆₀ alkenyl group; a C₂ to C₆₀ alkynyl group; a C₁ to C₆₀ haloalkyl group; a C₁ to C₆₀ alkoxy group; a C₆ to C₆₀ aryloxy group; a C₁ to C₆₀ alkylthioxy group; a C₆ to C₆₀ arylthioxy group; a C₁ to C₆₀ alkylsulfoxy group; a C₆ to C₆₀ arylsulfoxy group; a C₃ to C₆₀ cycloalkyl group; a C₂ to C₆₀ heterocycloalkyl group; a C₆ to C₆₀ aryl group; a C₂ to C₆₀ heteroaryl group; —SiRR′R″; —P(═O)RR′; and —NRR′, or a substituent in which two or more of substituents selected from the above-exemplified substituents are connected, wherein R, R′ and R″ are independently a substituent selected from hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not shown in a chemical formula or a compound structure”, it means that a hydrogen atom is bonded to a carbon atom. However, since deuterium (²H) is an isotope of hydrogen, some of hydrogen atoms may be deuterium.

In an exemplary embodiment of the present application, “when a substituent is not shown in a chemical formula or a compound structure”, it may mean that all possible substituents are hydrogen or deuterium. That is to say, some of hydrogen atoms may be deuterium, which is an isotope of hydrogen, and the content of deuterium may be 0% to 100%.

In an exemplary embodiment of the present application, “when a substituent is not shown in a chemical formula or a compound structure”, hydrogen and deuterium may be used interchangeably unless deuterium is excluded explicitly, such as, the content of deuterium is 0%, the content of hydrogen is 100%, all the substituents are hydrogen, etc.

In an exemplary embodiment of the present application, deuterium is an isotope of hydrogen having a deuteron consisting of one proton and one neutron as a nucleus. It may be expressed as hydrogen-2, and may be represented with the element symbol D or ²H.

In an exemplary embodiment of the present application, the isotope is an atom having the same atomic number (Z) but a different mass number (A). It may be interpreted as an element having the same number of protons but a different number of neutrons.

In an exemplary embodiment of the present application, if the total number of substituents that a compound can have is defined as T1 and the number of a specific substituent is defined as T2, the content of the specific substituent, T %, may be defined as T2/T1×100=T %.

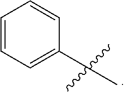 [SMILES: *c1ccccc1]

That is to say, for example, if a phenyl group represented by has a deuterium content of 20%, it means that the total number of substituents that the phenyl group can have (T1) is 5, and 1 (T2) out of them is deuterium (T %=20%). That is to say, the phenyl group having a deuterium content of 20% may be represented by the following structural formulas.

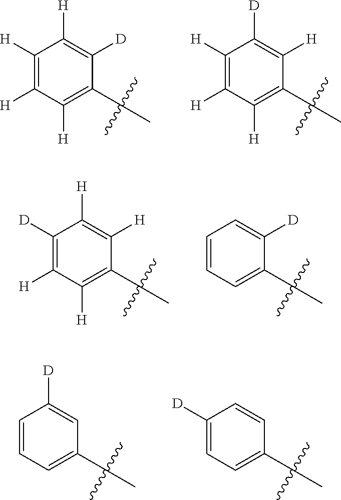 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In another exemplary embodiment of the present application, “a phenyl group having a deuterium content of 0%” may mean a phenyl group not containing a deuterium atom, i.e., having 5 hydrogen atoms.

In the present specification, the halogen may be fluorine, chlorine, bromine or iodine. In the present specification, the alkyl group includes a C₁ to C₆₀ linear or branched alkyl group, and it may be further substituted with other substituents. The alkyl group may have 1 to 60, specifically 1 to 40, more specifically, 1 to 20, carbon atoms. Specific examples include a methyl group, an ethyl group, a propyl group, a n-propyl group, an isopropyl group, a butyl group, a n-butyl group, an isobutyl group, a tert-butyl group, a sec-butyl group, a 1-methyl-butyl group, a 1-ethyl-butyl group, a pentyl group, a n-pentyl group, an isopentyl group, a neopentyl group, a tert-pentyl group, a hexyl group, a n-hexyl group, a 1-methylpentyl group, a 2-methylpentyl group, a 4-methyl-2-pentyl group, a 3,3-dimethylbutyl group, a 2-ethylbutyl group, heptyl group, a n-heptyl group, a 1-methylhexyl group, an octyl group, a n-octyl group, a tert-octyl group, a 1-methylheptyl group, a 2-ethylhexyl group, a 2-propylpentyl group, a n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group,

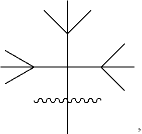 [SMILES: *C(C(C)(C)C)(C(C)(C)C)C(C)(C)C]

etc., although not being limited thereto.

In the present specification, the alkenyl group includes a C₂ to C₆₀ linear or branched alkenyl group, and it may be further substituted with other substituents. The alkenyl group may have 2 to 60, specifically 2 to 40, more specifically, 2 to 20, carbon atoms. Specific examples include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, etc., although not being limited thereto.

In the present specification, the alkynyl group includes a C₂ to C₆₀ linear or branched alkynyl group, and it may be further substituted with other substituents. The alkynyl group may have 2 to 60, specifically 2 to 40, more specifically, 2 to 20, carbon atoms.

In the present specification, the haloalkyl group refers to an alkyl group substituted with a halogen group. Specific examples include —CF₃, —CF₂CF₃, etc., although not being limited thereto.

In the present specification, the alkoxy group is represented by —O(R101), wherein R101 may be the alkyl group exemplified above.

In the present specification, the aryloxy group is represented by —O(R102), wherein R102 may be the aryl group exemplified above.

In the present specification, the alkylthioxy group is represented by —S(R103), wherein R103 may be the alkyl group exemplified above.

In the present specification, the arylthioxy group is represented by —S(R104), wherein R104 may be the aryl group exemplified above.

In the present specification, the alkylsulfoxy group is represented by —S(═O)₂(R105), wherein R105 may be the alkyl group exemplified above.

In the present specification, the arylsulfoxy group is represented by —S(═O)₂(R106), wherein R106 may be the aryl group exemplified above.

In the present specification, the cycloalkyl group includes a C₃ to C₆₀ monocyclic or polycyclic cycloalkyl group, and it may be further substituted with other substituents. Here, the polycyclic group means a group in which the cycloalkyl group is directly connected to or condensed with another ring group. Here, the another ring group may be a cycloalkyl group, or other ring groups, e.g., a heterocycloalkyl group, an aryl group, a heteroaryl group, etc. The cycloalkyl group may have 3 to 60, specifically 3 to 40, more specifically 5 to 20, carbon atoms. Specific examples include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, etc., although not being limited thereto.

In the present specification, the heterocycloalkyl group includes a C₂ to C₆₀ monocyclic or polycyclic heterocycloalkyl group containing O, S, Se, N or Si as a heteroatom, and it may be further substituted with other substituents. Here, the polycyclic group means a group in which the heterocycloalkyl group is directly connected to or condensed with another ring group. Here, the another ring group may be a heterocycloalkyl group, or other ring groups, e.g., a cycloalkyl group, an aryl group, a heteroaryl group, etc. The heterocycloalkyl group may have 2 to 60, specifically 2 to 40, more specifically 3 to 20, carbon atoms.

In the present specification, the aryl group includes a C₆ to C₆₀ monocyclic or polycyclic, and it may be further substituted with other substituents. Here, the polycyclic group means a group in which the aryl group is directly connected to or condensed with another ring group. Here, the another ring group may be an aryl group, or other ring groups, e.g., a cycloalkyl group, a heterocycloalkyl group, a heteroaryl group, etc. The aryl group includes a spiro group. The aryl group may have 6 to 60, specifically 6 to 40, more specifically 6 to 25, carbon atoms. Specific examples of the aryl group include a phenyl group, a biphenyl group, a terphenyl group, a naphthyl group, an anthryl group, a chrysenyl group, a phenanthryl group, a perylenyl group, a fluoranthenyl group, a triphenylenyl group, a phenalenyl group, a pyrenyl group, a tetracenyl group, a pentacenyl group, a fluorenyl group, an indenyl group, an acenaphtylenyl group, a benzofluorenyl group, a spirobifluorenyl group, a 2,3-dihydro-1H-indenyl group, a condensed ring group thereof, etc., although not being limited thereto.

In the present specification, the terphenyl group may be selected from the following structures.

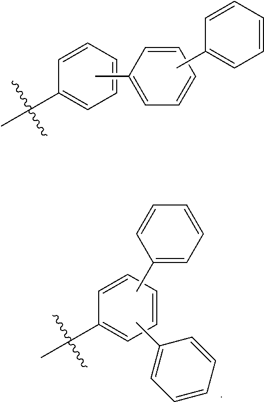

In the present specification, the fluorenyl group may be substituted, and adjacent substituents may be bonded to each other to form a ring.

When the fluorenyl group is substituted, it may be one of the following structures, although not being limited thereto

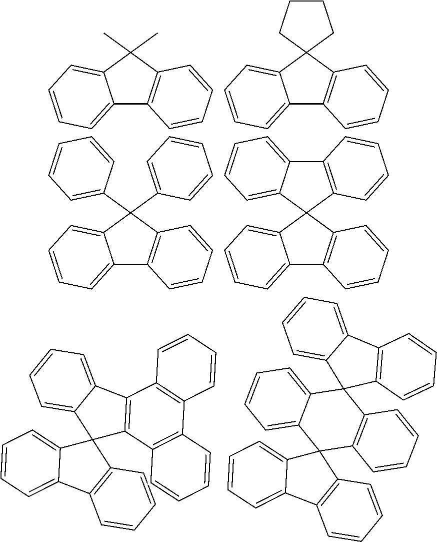

In the present specification, the heteroaryl group includes a C₂ to C₆₀ monocyclic or polycyclic heteroaryl group containing S, O, Se, N or Si as a heteroatom, and it may be further substituted with other substituents. Here, the polycyclic group means a group in which the heteroaryl group is directly connected to or condensed with another ring group. Here, the another ring group may be a heteroaryl group, or other ring groups, e.g., a cycloalkyl group, a heterocycloalkyl group, an aryl group, etc. The heteroaryl group may have 2 to 60, specifically 2 to 40, more specifically 3 to 25, carbon atoms. Specific examples of the heteroaryl group include a pyridine group, a pyrrole group, a pyrimidine group, a pyridazine group, a furan group, a thiophene group, an imidazolyl group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group a naphthyridine group, an acridine group, a phenanthridine group, an imidazopyridine group, a diazanaphthalene group, a triazaindene group, an indole group, an indolizine group, a benzothiazole group, a benzoxazole group, a benzimidazolyl group, a benzothiophene group, a benzofuran group, a dibenzothiophene group, a dibenzofuran group, a carbazole group, a benzocarbazole group, a dibenzocarbazole group, a phenazine group, a dibenzosilole group, a spirobi (dibenzosilole) group, a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, etc., although not being limited thereto.

In the present specification, when the substituent is a carbazole group, the substituent is bonded to the nitrogen or carbon of carbazole.

In the present specification, when the carbazole group is a carbazole group, an additional substituent may be substituted at the nitrogen or carbon of carbazole.

In the present specification, the benzocarbazole group may be one of the following structures.

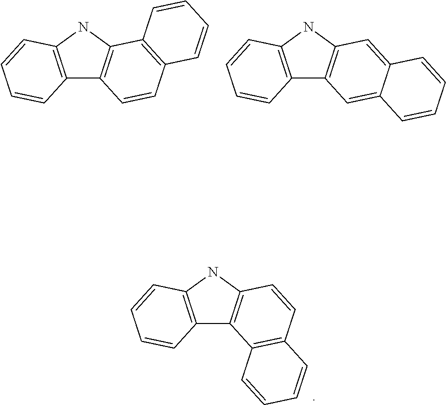

In the present specification, the dibenzocarbazole group may be one of the following structures.

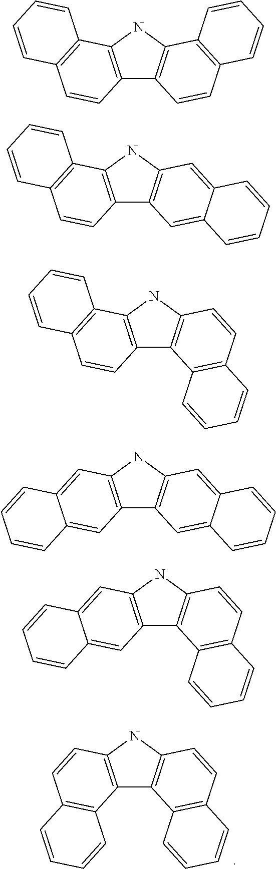

In the present specification, the naphthobenzofuran group may be one of the following structures.

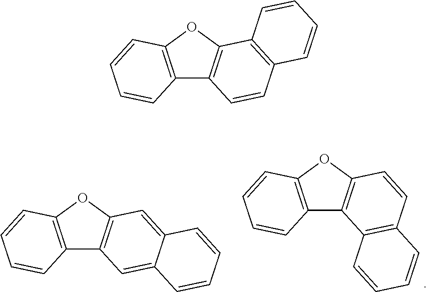

In the present specification, the naphthobenzothiophene group may be one of the following structures.

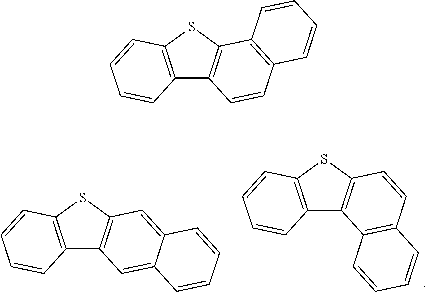

In the present specification, the aliphatic ring group includes a C₃ to C₆₀ monocyclic or polycyclic aliphatic ring group, and it may be further substituted with other substituents. Here, the polycyclic group means a group in which the cycloalkyl group is directly connected to or condensed with another ring group. Here, the another ring group may be a cycloalkyl group, or other ring groups, e.g., a heterocycloalkyl group, an aryl group, a heteroaryl group, etc. The cycloalkyl group may have 3 to 60, specifically 3 to 40, more specifically 5 to 20, carbon atoms. Specific examples of the aliphatic ring group include a cyclopropyl group, a cyclobutyl group, a cyclopentyl group, a cyclohexyl group, a cycloheptyl group, a cyclooctyl group, a cyclopentene group, a cyclohexene group, a norbornyl group, benzonorbornyl group, an adamantyl group, a spiro[4.5]decanyl group, a tetrahydronaphthyl group, an indanyl group, etc., although not being limited thereto.

In the present specification, the aliphatic ring group may be any one of the following structures, although not being limited thereto.

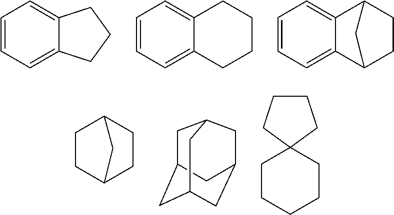

In the present specification, the silyl group is a substituent containing Si, wherein the Si atom is directly connected as a radical. It may be represented by —Si(R107)(R108)(R109), wherein R107 to R109, which are identical or different, may independently be at least one substituent selected from hydrogen; deuterium; a halogen group; an alkyl group; an alkenyl group; an alkoxy group; a cycloalkyl group; a heterocycloalkyl group; an aryl group; and a heteroaryl group.

Specific examples of the silyl group include

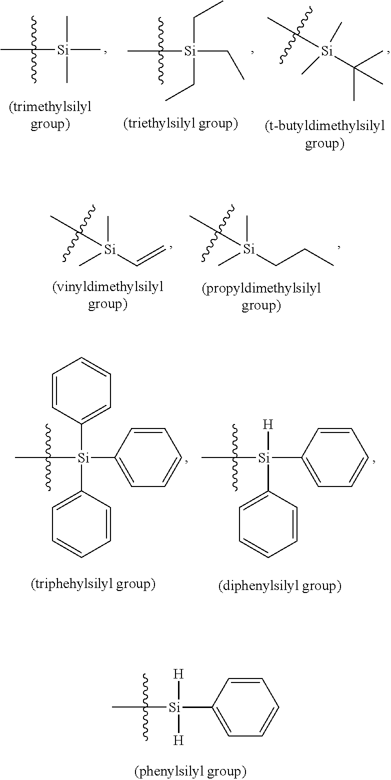

etc., although not being limited thereto.

In the present specification, the phosphine oxide group is represented by —P(═O)(R110)(R111), wherein R110 and R111, which are identical or different, may independently be at least one substituent selected from hydrogen; deuterium; a halogen group; an alkyl group; an alkenyl group; an alkoxy group; a cycloalkyl group; a heterocycloalkyl group; an aryl group; and a heteroaryl group. Specifically, it may be substituted with an alkyl group or an aryl group, and the alkyl group and the aryl group may be those exemplified above. For example, the phosphine oxide group may be a dimethylphosphine oxide group, a diphenylphosphine oxide group, a dinaphthylphosphine oxide group, etc., although not being limited thereto.

In the present specification, the amine group is represented by —N(R112)(R113), wherein R112 and R113, which are identical or different, may independently be at least one substituent selected from hydrogen; deuterium; a halogen group; an alkyl group; an alkenyl group; an alkoxy group; a cycloalkyl group; a heterocycloalkyl group; an aryl group; and a heteroaryl group. The amine group may be selected from a group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and may have 1 to 30 carbon atoms, although not being specially limited thereto. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, etc., although not being limited thereto.

In the present specification, the foregoing description of the aryl group applies to the arylene group, except that it is a divalent group.

In the present specification, the foregoing description of the heteroaryl group applies to the heteroarylene group, except that it is a divalent group.

In the present specification, the “adjacent” group may refer to a substituent substituted on an atom directly connected to the atom on which the substituent is substituted, a substituent stereostructurally closest to the substituent, or another substituent substituted on the atom on which the substituent is substituted. For example, two substituents substituted at the ortho positions in a benzene ring and two substituents substituted at the same carbon in an aliphatic ring may be interpreted as “adjacent” groups.

Hydrocarbon rings and hetero rings that can be formed by adjacent groups include aliphatic hydrocarbon rings, aromatic hydrocarbon rings, aliphatic hetero rings and aromatic hetero rings, and the rings may be the structures exemplified above as the cycloalkyl group, the aryl group, the heterocycloalkyl group and the heteroaryl group, except that they are not monovalent groups.

In an exemplary embodiment of the present application, a group not represented as a substituent; or a group represented as hydrogen may be substituted with deuterium. That is to say, they may be substituted with hydrogen; or deuterium.

In general, a compound consisting only of hydrogen exhibits thermodynamic behaviors different from those of a compound substituted with deuterium. This is because the mass of the deuterium atom is twice that of hydrogen. Due to the difference in the atomic mass, deuterium has a smaller vibrational energy.

In addition, the bond dissociation energy of a bond between carbon and deuterium is higher than the bond dissociation energy of a bond between carbon and hydrogen. Therefore, the substitution with deuterium increases the thermal stability of the molecule, which improves the lifetime of a device using the same.

When a compound is deposited on a silicon wafer, a material containing deuterium tends to be packed with a smaller intermolecular distance. Furthermore, when the thin film surface is observed with an atomic force microscope (AFM), it can be confirmed that a thin film prepared from a compound containing deuterium is deposited more uniformly without aggregation.

In an exemplary embodiment of the present application, there is provided a compound represented by Chemical Formula 1.

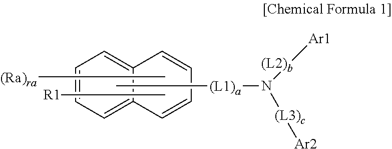

In Chemical Formula 1,
R1 is a substituted or unsubstituted C₁ to C₆₀ branched alkyl group,
L1 is a substituted or unsubstituted C₆ to C₆₀ arylene group,
a is an integer from 1 to 3, wherein if a is 2 or greater, L1's are identical or different,
L2 and L3, which are identical or different, are independently a direct bond; or a substituted or unsubstituted C₆ to C₆₀ arylene group,
b and c are respectively an integer from 0 to 3, wherein if b is 2 or greater, L2's are identical or different, and if c is 2 or greater, L3's are identical or different,
Ra is hydrogen; deuterium; a halogen group; a cyano group; a substituted or unsubstituted C₁ to C₆₀ alkyl group; a substituted or unsubstituted C₆ to C₆₀ aryl group; or a substituted or unsubstituted C₂ to C₆₀ heteroaryl group,
ra is an integer from 0 to 6, wherein if ra is 2 or greater, Ra's are identical or different,
with the proviso that L1 and R1 are not bonded at para positions,
Ar1 and Ar2, which are identical or different, are independently a substituted or unsubstituted C₄ to C₆₀ monocyclic or polycyclic aliphatic ring group; a substituted or unsubstituted C₆ to C₆₀ aryl group consisting of one or more benzene ring; a substituted or unsubstituted C₂ to C₆₀ tricyclic heteroaryl group; or Structural Formula A,

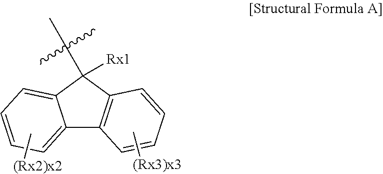

in Structural Formula A,

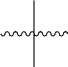

is a moiety connected to Chemical Formula 1,
Rx1 is a substituted or unsubstituted C₁ to C₆₀ alkyl group; a substituted or unsubstituted C₄ to C₆₀ monocyclic or polycyclic aliphatic ring group; or a substituted or unsubstituted C₆ to C₆₀ aryl group,
Rx2 and Rx3 are hydrogen; deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group, or two or more adjacent substituents are bonded to each other to form a substituted or unsubstituted ring, and
x2 and x3 are integers from 0 to 4, wherein if x2 and x3 are respectively 2 or greater, the substituents in the parentheses are identical or different.

The “substituted or unsubstituted C₆ to C₆₀ aryl group consisting of one or more benzene ring” refers to an aryl-based substituent consisting only of a benzene ring, such as a phenyl group, a biphenyl group, a terphenyl group, a naphthyl group, a triphenylene group or a phenanthrene group, and does not refer to an aryl-based substituent containing both a benzene ring and a hydrocarbon ring, such as a fluorenyl group.

The expression that “L1 and R1 are not bonded at para positions” means that the case where L1 and R1 are bonded as in the following structure (e.g., the substituents R1 and L1 are bonded at C-1 and C-4 positions of the same benzene moiety) is excluded.

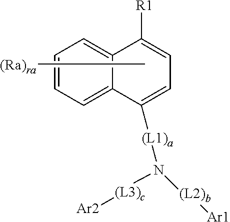

The compound represented by Chemical Formula 1 of the present application can have appropriate hole mobility and electron suppression ability in a device since L1 is a substituted or unsubstituted C₆ to C₆₀ arylene group. In particular, if L1 is a direct bond, the effect of suppressing deterioration of the hole transport material caused by electrons transferred to the hole transport layer may not be good because the LUMO Level is lower as compared to when it is not a direct bond.

In addition, since L1 and R1 are bonded at specific positions in the core, hole mobility may be controlled appropriately. In particular, when they are bonded at para positions, the overall charge balance required for the device cannot be stabilized since charge transfer is faster than required due to the short intermolecular distance.

In an exemplary embodiment of the present application, Chemical Formula 1 may be represented by any one of Chemical Formulas 1-1 to 1-13.

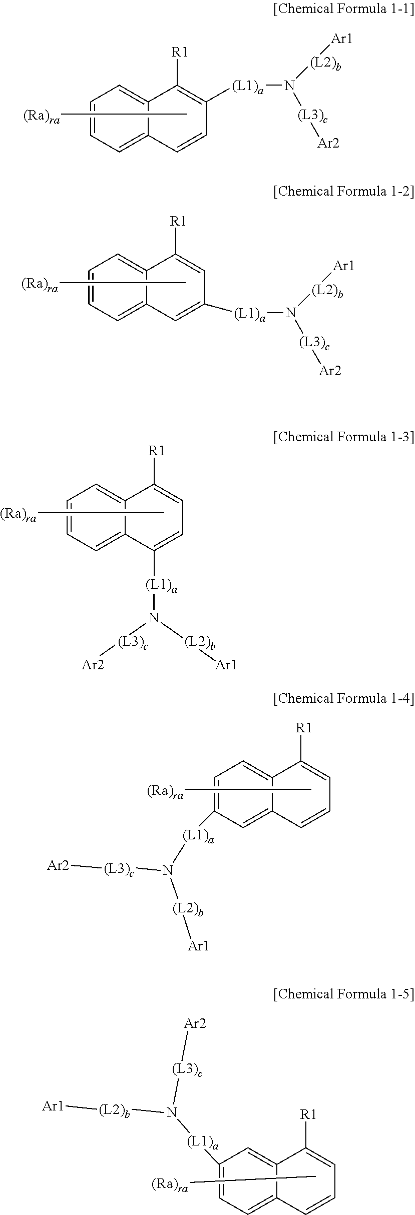

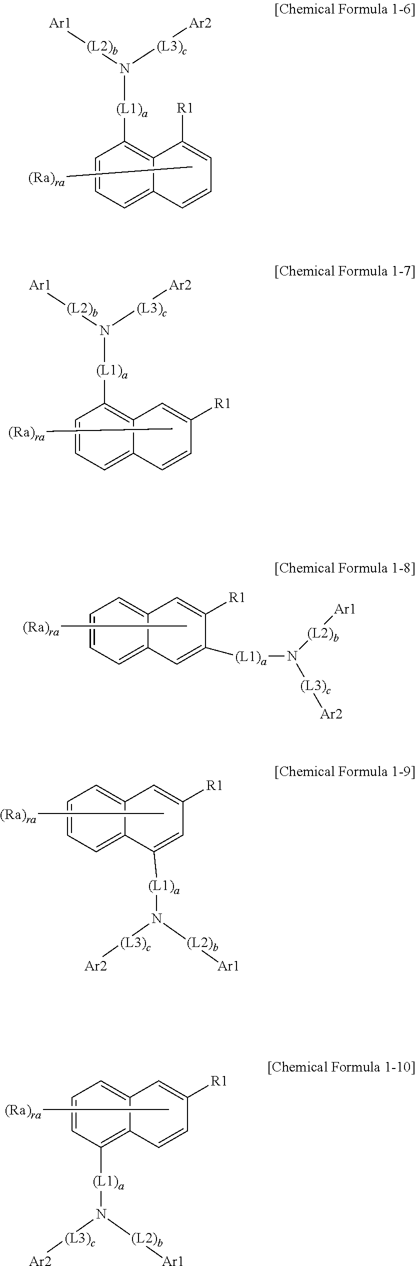

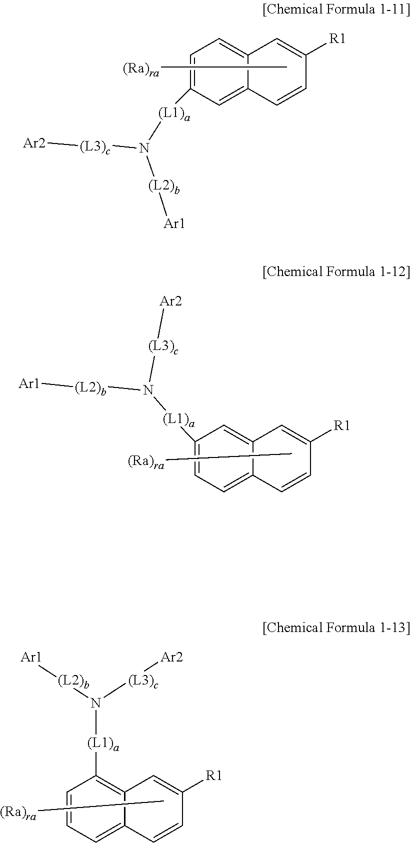

In Chemical Formulas 1-1 to 1-13,
L1, L2, L3, Ar1, Ar2, R1, Ra, a, b, c and ra are the same as defined in Chemical Formula 1.

In Chemical Formulas 1-1 to 1-13, R1 may be represented by Chemical Formula H-1.

In an exemplary embodiment of the present application,

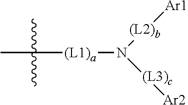

of Chemical Formula 1 may be represented by any one of Chemical Formulas A to E.

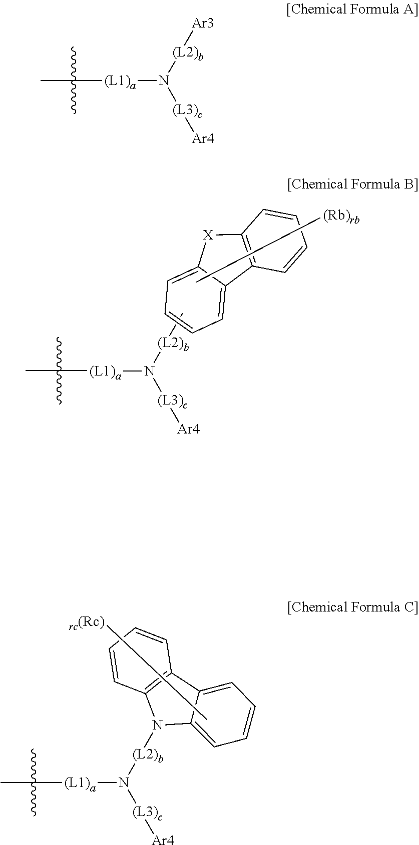

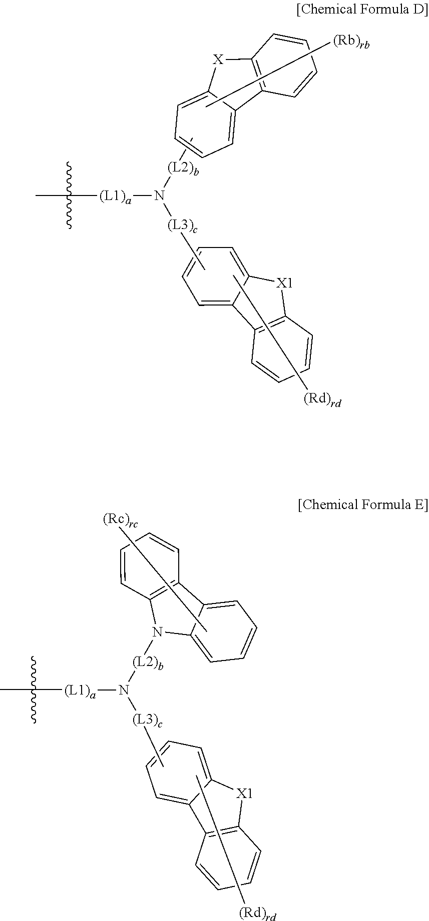

In Chemical Formulas A to E,
L1, L2, L3, a, b and c are the same as defined in Chemical Formula 1,
Ar3 and Ar4, which are identical or different, are independently a substituted or unsubstituted cyclopentyl group; a substituted or unsubstituted cyclohexyl group; a substituted or unsubstituted adamantyl group; a substituted or unsubstituted norbornyl group; a substituted or unsubstituted benzonorbornyl group; a substituted or unsubstituted indanyl group; a substituted or unsubstituted tetrahydronaphthyl group; a substituted or unsubstituted spiro[4.5]decanyl group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; or Structural Formula A,
X is O, S or NR,
R is hydrogen; deuterium; a substituted or unsubstituted C₁ to C₆₀ alkyl group; or a substituted or unsubstituted C₆ to C₆₀ aryl group,
X1 is O or S,
Rb, Rc and Rd, which are identical or different, are independently hydrogen; deuterium; a halogen group; a cyano group; a substituted or unsubstituted C₁ to C₆₀ alkyl group; a substituted or unsubstituted C₆ to C₆₀ aryl group; or a substituted or unsubstituted C₂ to C₆₀ heteroaryl group,
rb and rd are respectively an integer from 0 to 7, wherein if rb is 2 or greater, Rb's are identical or different, and if rd is 2 or greater, Rd's are identical or different, and
rc is an integer from 0 to 8, and if rc is 2 or greater, Rc's are identical or different.

In an exemplary embodiment of the present application, Ar1 and Ar2, which are identical or different, may independently be a substituted or unsubstituted cyclopentyl group; a substituted or unsubstituted cyclohexyl group; a substituted or unsubstituted adamantyl group; a substituted or unsubstituted norbornyl group; a substituted or unsubstituted benzonorbornyl group; a substituted or unsubstituted indanyl group; a substituted or unsubstituted tetrahydronaphthyl group; a substituted or unsubstituted spiro[4.5]decanyl group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; a substituted or unsubstituted dibenzofuran group; a substituted or unsubstituted dibenzothiophene group; a substituted or unsubstituted carbazole group; or Structural Formula A, and

Structural Formula A may be any one of Structural Formulas A-1 to A-4.

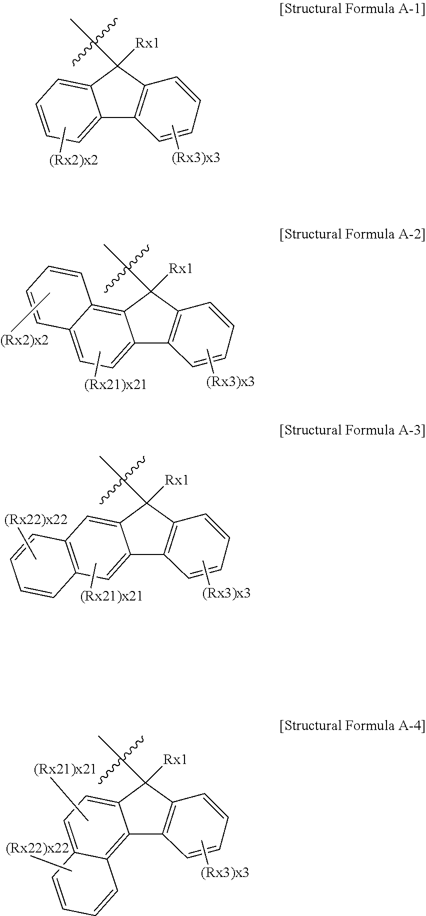

Rx21 and Rx22, which are identical or different, are independently, hydrogen; deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group,
x21 is an integer from 0 to 2, and if x21 is 2, the substituents in the parentheses are identical or different,
x22 is an integer from 0 to 4, and if x22 is 2 or greater, the substituents in the parentheses are identical or different, and
Rx1 to Rx3, x2 and x3 are the same as defined in Structural Formula A.

In an exemplary embodiment of the present application, R1 may be represented by Chemical Formula H-1.

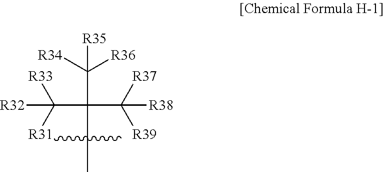

In Chemical Formula H-1,
R31 to R39, which are identical or different, are independently hydrogen; or deuterium.

In another exemplary embodiment, R1 may be a tert-butyl group substituted or unsubstituted with deuterium.

In another exemplary embodiment, R1 may be a tert-butyl group;

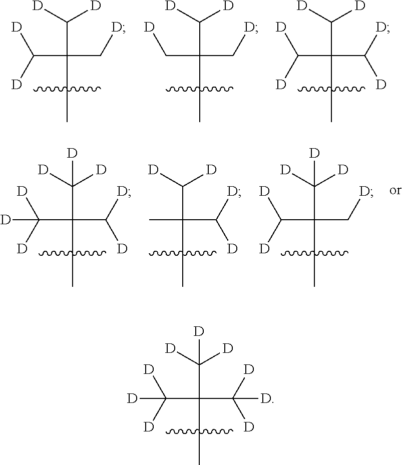

In another exemplary embodiment, R1 may be

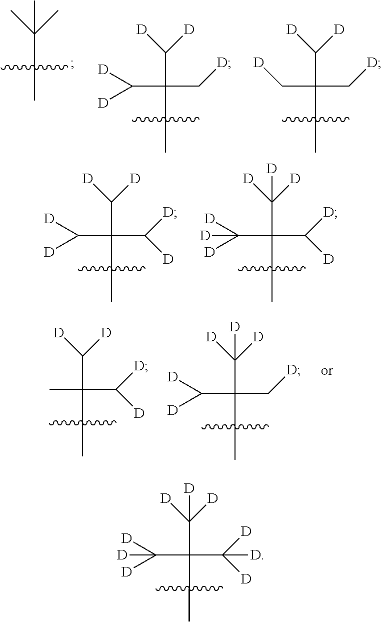

In an exemplary embodiment of the present application, L1 may be a substituted or unsubstituted C₆ to C₄₀ arylene group.

In another exemplary embodiment, L1 may be a substituted or unsubstituted C₆ to C₃₀ arylene group.

In another exemplary embodiment, L1 may be a substituted or unsubstituted phenylene group; a substituted or unsubstituted biphenylene group; a substituted or unsubstituted naphthylene group; or a substituted or unsubstituted phenanthrenylene group.

In another exemplary embodiment, L1 may be a phenylene group substituted or unsubstituted with deuterium; a biphenylene group substituted or unsubstituted with deuterium; a naphthylene group substituted or unsubstituted with deuterium; or a phenanthrenylene group substituted or unsubstituted with deuterium.

In an exemplary embodiment of the present application, a may be an integer 1 or 2, and if a is 2, L1's may be identical or different.

In another exemplary embodiment, a may be 2, and L1's may be identical or different.

In another exemplary embodiment, a may be 1.

In an exemplary embodiment of the present application, L2 and L3 may respectively be a direct bond; or a substituted or unsubstituted C₆ to C₄₀ arylene group.

In another exemplary embodiment, L2 and L3 may respectively be a direct bond; or a substituted or unsubstituted C₆ to C₃₀ arylene group.

In another exemplary embodiment, L2 and L3 may respectively be a direct bond; a substituted or unsubstituted phenylene group; a substituted or unsubstituted biphenylene group; a substituted or unsubstituted naphthylene group; or a substituted or unsubstituted phenanthrenylene group.

In another exemplary embodiment, L2 and L3 may respectively be a direct bond; a phenylene group substituted or unsubstituted with deuterium; a biphenylene group substituted or unsubstituted with deuterium; a naphthylene group substituted or unsubstituted with deuterium; or a phenanthrenylene group substituted or unsubstituted with deuterium.

In an exemplary embodiment of the present application, b and c may be integers from 0 to 2, wherein if b is 2, L2's may be identical or different, and if c is 2, L3's may be identical or different.

In another exemplary embodiment, b and c may be 2, and L2's and L3's may be identical or different.

In another exemplary embodiment, b and c may be 0 or 1.

In an exemplary embodiment of the present application, Ar1 and Ar2, which are identical or different, may independently be a substituted or unsubstituted aliphatic ring group; a substituted or unsubstituted C₆ to C₄₀ aryl group consisting of one or more benzene ring; or a substituted or unsubstituted C₂ to C₄₀ tricyclic heteroaryl group; or Structural Formula A.

In another exemplary embodiment, Ar1 and Ar2, which are identical or different, may independently be a substituted or unsubstituted aliphatic ring group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; a substituted or unsubstituted C₂ to C₄₀ tricyclic heteroaryl group; or Structural Formula A.

In another exemplary embodiment, Ar1 and Ar2, which are identical or different, may independently be a substituted or unsubstituted aliphatic ring group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; substituted or unsubstituted C₂ to C₃₀ tricyclic heteroaryl group; or Structural Formula A.

In another exemplary embodiment, Ar1 and Ar2, which are identical or different, may independently be a substituted or unsubstituted cyclopentyl group; a substituted or unsubstituted cyclohexyl group; a substituted or unsubstituted cyclopentyl group condensed with a phenyl group; a substituted or unsubstituted cyclohexyl group condensed with a phenyl group; a substituted or unsubstituted adamantyl group; a substituted or unsubstituted norbornyl group; a substituted or unsubstituted benzonorbornyl group; a substituted or unsubstituted indanyl group; a substituted or unsubstituted tetrahydronaphthyl group; a substituted or unsubstituted spiro[4.5]decanyl group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; a substituted or unsubstituted dibenzofuran group; a substituted or unsubstituted dibenzothiophene group; a substituted or unsubstituted carbazole group; or Structural Formula A.

In another exemplary embodiment, Ar1 and Ar2, which are identical or different, may independently be a cyclopentyl group substituted or unsubstituted with deuterium; a cyclohexyl group substituted or unsubstituted with deuterium; an adamantyl group substituted or unsubstituted with deuterium; a norbornyl group substituted or unsubstituted with deuterium; a benzonorbornyl group substituted or unsubstituted with deuterium; an indanyl group substituted or unsubstituted with deuterium; a tetrahydronaphthyl group substituted or unsubstituted with deuterium; a spiro[4.5]decanyl group substituted or unsubstituted with deuterium; a phenyl group substituted or unsubstituted with deuterium; a biphenyl group substituted or unsubstituted with deuterium; a terphenyl group substituted or unsubstituted with deuterium; a naphthyl group substituted or unsubstituted with deuterium; a triphenylene substituted or unsubstituted with deuterium; a phenanthrene substituted or unsubstituted with deuterium; a dibenzofuran group substituted or unsubstituted with deuterium; a dibenzothiophene substituted or unsubstituted with deuterium; a carbazole substituted or unsubstituted with deuterium; or Structural Formula A.

In another exemplary embodiment, Ar1 and Ar2, which are identical or different, may independently be a cyclopentyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a cyclohexyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; an adamantyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a norbornyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a benzonorbornyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; an indanyl group unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a tetrahydronaphthyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a spiro[4.5]decanyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a phenyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a biphenyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a terphenyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a naphthyl group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a triphenylene group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a phenanthrene group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a dibenzofuran group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a dibenzothiophene group substituted or unsubstituted with deuterium, a C₁ to C₆₀ branched alkyl group substituted or unsubstituted with deuterium, a C₄ to C₆₀ cycloalkyl group substituted or unsubstituted with deuterium or a naphthyl group; a carbazole group substituted or unsubstituted with deuterium, a C to C₆₀ branched alkyl group substituted or unsubstituted with deuterium, a C₄ to C₆₀ cycloalkyl group substituted or unsubstituted with deuterium or a naphthyl group; or Structural Formula A.

In another exemplary embodiment, Ar1 and Ar2, which are identical or different, may independently be a cyclopentyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

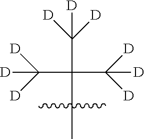 [SMILES: *C(C([2H])([2H])[2H])(C([2H])([2H])[2H])C([2H])([2H])[2H]]

or deuterium; a cyclohexyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, a an adamantyl group substituted or unsubstituted with deuterium or a naphthyl group substituted or unsubstituted with

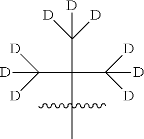 [SMILES: *C(C([2H])([2H])[2H])(C([2H])([2H])[2H])C([2H])([2H])[2H]]

or deuterium; an adamantyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

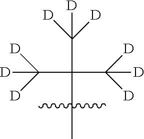 [SMILES: *C(C([2H])([2H])[2H])(C([2H])([2H])[2H])C([2H])([2H])[2H]]

or deuterium; a norbornyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

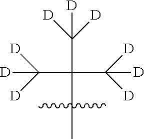 [SMILES: *C(C([2H])([2H])[2H])(C([2H])([2H])[2H])C([2H])([2H])[2H]]

or deuterium; a benzonorbornyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

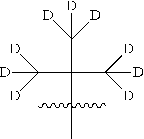 [SMILES: *C(C([2H])([2H])[2H])(C([2H])([2H])[2H])C([2H])([2H])[2H]]

or deuterium; an indanyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

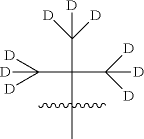 [SMILES: *C(C([2H])([2H])[2H])(C([2H])([2H])[2H])C([2H])([2H])[2H]]

or deuterium; a tetrahydronaphthyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

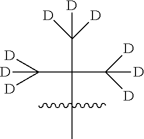 [SMILES: *C(C([2H])([2H])[2H])(C([2H])([2H])[2H])C([2H])([2H])[2H]]

or deuterium; a spiro[4.5]decanyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

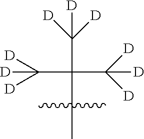 [SMILES: *C(C([2H])([2H])[2H])(C([2H])([2H])[2H])C([2H])([2H])[2H]]

or deuterium; a phenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

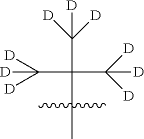 [SMILES: *C(C([2H])([2H])[2H])(C([2H])([2H])[2H])C([2H])([2H])[2H]]

or deuterium; a biphenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

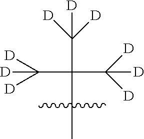 [SMILES: *C(C([2H])([2H])[2H])(C([2H])([2H])[2H])C([2H])([2H])[2H]]

or deuterium; a terphenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

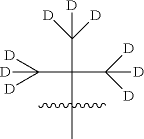 [SMILES: *C(C([2H])([2H])[2H])(C([2H])([2H])[2H])C([2H])([2H])[2H]]

or deuterium; a naphthyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

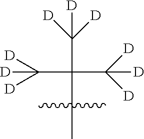 [SMILES: *C(C([2H])([2H])[2H])(C([2H])([2H])[2H])C([2H])([2H])[2H]]

or deuterium; a triphenylene substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

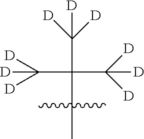 [SMILES: *C(C([2H])([2H])[2H])(C([2H])([2H])[2H])C([2H])([2H])[2H]]

or deuterium; a phenanthrene substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

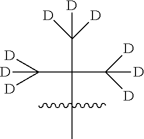 [SMILES: *C(C([2H])([2H])[2H])(C([2H])([2H])[2H])C([2H])([2H])[2H]]

or deuterium; a dibenzofuran group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

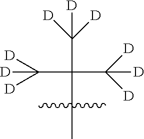 [SMILES: *C(C([2H])([2H])[2H])(C([2H])([2H])[2H])C([2H])([2H])[2H]]

or deuterium; a dibenzothiophene substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

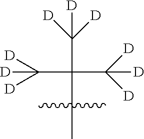 [SMILES: *C(C([2H])([2H])[2H])(C([2H])([2H])[2H])C([2H])([2H])[2H]]

or deuterium; a carbazole substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

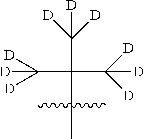 [SMILES: *C(C([2H])([2H])[2H])(C([2H])([2H])[2H])C([2H])([2H])[2H]]

or deuterium; or Structural Formula A.

In an exemplary embodiment of the present application, Ra may be hydrogen; deuterium; a halogen group; a cyano group; a substituted or unsubstituted C₁ to C₄₀ alkyl group; a substituted or unsubstituted C₆ to C₄₀ aryl group; or a substituted or unsubstituted C₂ to C₄₀ heteroaryl group.

In another exemplary embodiment, Ra may be hydrogen; deuterium; a halogen group; a cyano group; a substituted or unsubstituted C₁ to C₂₀ alkyl group; a substituted or unsubstituted C₆ to C₂₀ aryl group; or a substituted or unsubstituted C₂ to C₂₀ heteroaryl group.

In another exemplary embodiment, Ra may be hydrogen; deuterium; a halogen group; a cyano group; a substituted or unsubstituted methyl group; a substituted or unsubstituted propyl group; a substituted or unsubstituted butyl group; a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

In another exemplary embodiment, Ra may be hydrogen; deuterium; a halogen group; a cyano group; a methyl group; a propyl group; a butyl group; a phenyl group; a biphenyl group; a terphenyl group; a naphthyl group; a dibenzofuran group; or a dibenzothiophene group.

In another exemplary embodiment, Ra may be hydrogen; or deuterium. In an exemplary embodiment of the present application, ra may be an integer from 0 to 5, and if ra is 2 or greater, Ra's may be identical or different.

In another exemplary embodiment, ra may be an integer from 0 to 4, and if ra is 2 or greater, Ra's may be identical or different.

In another exemplary embodiment, ra may be an integer from 0 to 3, and if ra is 2 or greater, Ra's may be identical or different.

In another exemplary embodiment, ra may be an integer from 0 to 2, and if ra is 2, Ra's may be identical or different.

In another exemplary embodiment, ra may be 0 or 1.

In an exemplary embodiment of the present application, Rx1 may be a substituted or unsubstituted C₁ to C₆₀ alkyl group; a substituted or unsubstituted C₄ to C₆₀ monocyclic or polycyclic aliphatic ring group; or a substituted or unsubstituted C₆ to C₆₀ aryl group.

In another exemplary embodiment, Rx1 may be a substituted or unsubstituted C₁ to C₁₀ alkyl group; a substituted or unsubstituted C₄ to C₂₀ monocyclic or polycyclic aliphatic ring group; or a substituted or unsubstituted C₆ to C₃₀ aryl group.

In another exemplary embodiment, Rx1 may be a C₁ to C₁₀ alkyl group substituted or unsubstituted with deuterium or a substituted or unsubstituted C₁ to C₅ alkyl group or a substituted or unsubstituted C₆ to C₃₀ aryl group; a C₄ to C₂₀ monocyclic or polycyclic aliphatic ring group substituted or unsubstituted with deuterium or a substituted or unsubstituted C₁ to C₅ alkyl group or a substituted or unsubstituted C₆ to C₃₀ aryl group; or a C₆ to C₃₀ aryl group substituted or unsubstituted with deuterium or a substituted or unsubstituted C₁ to C₅ alkyl group or a substituted or unsubstituted C₆ to C₃₀ aryl group.

In another exemplary embodiment, Rx1 may be a substituted or unsubstituted methyl group; a substituted or unsubstituted tert-butyl group; a substituted or unsubstituted cyclopentyl group; a substituted or unsubstituted cyclohexyl group; substituted or unsubstituted norbornyl group; a substituted or unsubstituted adamantyl group; a substituted or unsubstituted phenyl group; a substituted or unsubstituted biphenyl group; a substituted or unsubstituted naphthyl group; or a substituted or unsubstituted phenanthrene group.

In another exemplary embodiment, Rx1 may be a methyl group substituted or unsubstituted with deuterium, a phenyl group or a tert-butyl group; a tert-butyl group substituted or unsubstituted with deuterium, a phenyl group or a tert-butyl group; a cyclopentyl group substituted or unsubstituted with deuterium, a phenyl group or a tert-butyl group; a cyclohexyl group substituted or unsubstituted with deuterium, a phenyl group or a tert-butyl group; a norbornyl group substituted or unsubstituted with deuterium, a phenyl group or a tert-butyl group; an adamantyl group substituted or unsubstituted with deuterium, a phenyl group or a tert-butyl group; a phenyl group substituted or unsubstituted with deuterium, a phenyl group or a tert-butyl group; a biphenyl group substituted or unsubstituted with deuterium, a phenyl group or a tert-butyl group; a naphthyl group substituted or unsubstituted with deuterium, a phenyl group or a tert-butyl group; or a phenanthrene group substituted or unsubstituted with deuterium, a phenyl group or a tert-butyl group.

In an exemplary embodiment of the present application, Rx2 and Rx3, which are identical or different, may independently be hydrogen; deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group, or two or more adjacent substituents may be bonded to each other to form a substituted or unsubstituted ring.

In another exemplary embodiment, Rx2 and Rx3, which are identical or different, may independently be hydrogen; deuterium; or a substituted or unsubstituted C₁ to C₆₀ alkyl group, or two or more adjacent substituents may be bonded to each other to form a substituted or unsubstituted ring.

In another exemplary embodiment, Rx2 and Rx3, which are identical or different, may independently be hydrogen; deuterium; or a substituted or unsubstituted C₆ to C₆₀ aryl group, or two or more adjacent substituents may be bonded to each other to form a substituted or unsubstituted ring.

In another exemplary embodiment, Rx2 and Rx3, which are identical or different, may independently be hydrogen; deuterium; or a substituted or unsubstituted C₄ to C₆₀ monocyclic or polycyclic aliphatic ring group, or two or more adjacent substituents may be bonded to each other to form a substituted or unsubstituted ring.

In another exemplary embodiment, Rx2 and Rx3, which are identical or different, may independently be hydrogen; deuterium; a cyano group; a C₁ to C₆₀ alkyl group substituted or unsubstituted with deuterium, a substituted or unsubstituted C₁ to C₁₀ alkyl group or a substituted or unsubstituted C₆ to C₁₈ aryl group; a C₄ to C₆₀ monocyclic or polycyclic aliphatic ring group substituted or unsubstituted with deuterium, a substituted or unsubstituted C₁ to C₁₀ alkyl group or a substituted or unsubstituted C₆ to C₁₈ aryl group; a C₆ to C₆₀ aryl group substituted or unsubstituted with deuterium, a substituted or unsubstituted C₁ to C₁₀ alkyl group or a substituted or unsubstituted C₆ to C₁₈ aryl group; or a substituted or unsubstituted C₂ to C₆₀ heteroaryl group, or two or more adjacent substituents may be bonded to each other to form a ring substituted or unsubstituted with deuterium, a substituted or unsubstituted C₁ to C₁₀ alkyl group or a substituted or unsubstituted C₆ to C₁₈ aryl group.

In another exemplary embodiment, Rx2 and Rx3, which are identical or different, may independently be hydrogen; deuterium; a C₆ to C₆₀ aryl group substituted or unsubstituted with deuterium, a substituted or unsubstituted C₁ to C₁₀ alkyl group or a substituted or unsubstituted C₆ to C₁₈ aryl group, or two or more adjacent substituents may be bonded to each other to form a ring substituted or unsubstituted with deuterium, a substituted or unsubstituted C₁ to C₁₀ alkyl group or a substituted or unsubstituted C₆ to C₁₈ aryl group.

In another exemplary embodiment, Rx2 and Rx3, which are identical or different, may independently be hydrogen; deuterium; a C₄ to C₆₀ monocyclic or polycyclic aliphatic ring group substituted or unsubstituted with deuterium, a substituted or unsubstituted C₁ to C₁₀ alkyl group or a substituted or unsubstituted C₆ to C₁₈ aryl group, or two or more adjacent substituents may be bonded to each other to form a ring substituted or unsubstituted with deuterium, a substituted or unsubstituted C₁ to C₁₀ alkyl group or a substituted or unsubstituted C₆ to C₁₈ aryl group.

In another exemplary embodiment, Rx2 and Rx3, which are identical or different, may independently be hydrogen; deuterium; a C₆ to C₆₀ aryl group substituted or unsubstituted with deuterium, a substituted or unsubstituted C₁ to C₁₀ alkyl group or a substituted or unsubstituted C₆ to C₁₈ aryl group, or two or more adjacent substituents may be bonded to each other to form a ring substituted or unsubstituted with deuterium, a substituted or unsubstituted C₁ to C₁₀ alkyl group or a substituted or unsubstituted C₆ to C₁₈ aryl group.

In another exemplary embodiment, Rx2 and Rx3, which are identical or different, may independently be hydrogen; deuterium; a cyano group; a substituted or unsubstituted methyl group; a substituted or unsubstituted tert-butyl group; a substituted or unsubstituted cyclopentyl group; a substituted or unsubstituted cyclohexyl group; a substituted or unsubstituted norbornyl group; a substituted or unsubstituted adamantyl group; a substituted or unsubstituted phenyl group; a substituted or unsubstituted biphenyl group; a substituted or unsubstituted terphenyl group; a substituted or unsubstituted naphthyl group; or a substituted or unsubstituted phenanthrene group, or two or more adjacent substituents may be bonded to each other to form a substituted or unsubstituted benzene ring.

In another exemplary embodiment, Rx2 and Rx3, which are identical or different, may independently be hydrogen; deuterium; a cyano group; a methyl group substituted or unsubstituted with deuterium, a tert-butyl group or a phenyl group; a tert-butyl group substituted or unsubstituted with deuterium, a tert-butyl group or a phenyl group; a cyclopentyl group substituted or unsubstituted with deuterium, a tert-butyl group or a phenyl group; a cyclohexyl group substituted or unsubstituted with deuterium, a tert-butyl group or a phenyl group; a norbornyl group substituted or unsubstituted with deuterium, a tert-butyl group or a phenyl group; an adamantyl group substituted or unsubstituted with deuterium, a tert-butyl group or a phenyl group; a phenyl group substituted or unsubstituted with deuterium, a tert-butyl group or a phenyl group; a naphthyl group substituted or unsubstituted with deuterium, a tert-butyl group or a phenyl group; or a phenanthrene group substituted or unsubstituted with deuterium, a tert-butyl group or a phenyl group, or two or more adjacent substituents may be bonded to each other to form a benzene ring substituted or unsubstituted with deuterium, a tert-butyl group or a phenyl group.

In an exemplary embodiment of the present application, Rx21 and Rx22, which are identical or different, may independently be hydrogen; deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group.

In another exemplary embodiment, Rx21 and Rx22, which are identical or different, may independently be hydrogen.

In another exemplary embodiment, Rx21 and Rx22, which are identical or different, may independently be hydrogen or deuterium.

In another exemplary embodiment, Rx21 and Rx22, which are identical or different, may independently be hydrogen or a cyano group.

In another exemplary embodiment, Rx21 and Rx22, which are identical or different, may independently be hydrogen or a substituted or unsubstituted C₁ to C₆₀ alkyl group.

In another exemplary embodiment, Rx21 and Rx22, which are identical or different, may independently be hydrogen; deuterium; a cyano group or a substituted or unsubstituted C₁ to C₆₀ alkyl group.

In another exemplary embodiment, Rx21 and Rx22, which are identical or different, may independently be hydrogen; deuterium; a cyano group or a substituted or unsubstituted C₁ to C₁₀ alkyl group.

In another exemplary embodiment, Rx21 and Rx22, which are identical or different, may independently be hydrogen; deuterium; a cyano group or a C₁ to C₆₀ alkyl group substituted or unsubstituted with deuterium.

In another exemplary embodiment, Rx21 and Rx22, which are identical or different, may independently be hydrogen; deuterium; a cyano group or a C₁ to C₁₀ alkyl group substituted or unsubstituted with deuterium.

In another exemplary embodiment, Rx21 and Rx22, which are identical or different, may independently be hydrogen; deuterium; a cyano group or a substituted or unsubstituted methyl group; or a substituted or unsubstituted tert-butyl group.

In another exemplary embodiment, Rx21 and Rx22, which are identical or different, may independently be hydrogen; deuterium; a cyano group or a methyl group substituted or unsubstituted with deuterium; or a tert-butyl group substituted or unsubstituted with deuterium.

In an exemplary embodiment of the present application, x2, x22 and x3 may be integers from 0 to 4, wherein if x2, x22 and x3 are respectively 2 or greater, the substituents in the parentheses are identical or different.

In another exemplary embodiment, x2, x22 and x3 may be 0.

In another exemplary embodiment, x2, x22 and x3 may be 1.

In another exemplary embodiment, x2, x22 and x3 may be 0 or 1.

In another exemplary embodiment, x2, x22 and x3 may be 0 to 2, and if x2, x22 and x3 are respectively 2, the substituents in the parentheses are identical or different.

In an exemplary embodiment of the present application, x21 may be an integer from 0 to 2, and if x21 and x31 are respectively 2, the substituents in the parentheses are identical or different.

In another exemplary embodiment, x21 may be 0.

In another exemplary embodiment, x21 may be 1.

In another exemplary embodiment, x21 may be 0 or 1.

In an exemplary embodiment of the present application, Ar3 and Ar4, which are identical or different, may independently be a cyclopentyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a cyclohexyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; an adamantly group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a norbornyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a benzonorbornyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; an indanyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a tetrahydronaphthyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a spiro[4.5]decanyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a phenyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a biphenyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a terphenyl group substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a naphthyl group substituted or unsubstituted with deuterium, a phenyl group substituted or unsubstituted with deuterium or a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl group substituted or unsubstituted with deuterium; a triphenylene substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl substituted or unsubstituted with deuterium; a phenanthrene substituted or unsubstituted with deuterium, a C₁ to C₆₀ branched alkyl group substituted or unsubstituted with deuterium, a C₄ to C₆₀ cycloalkyl group substituted or unsubstituted with deuterium, a C₄ to C₆₀ monocyclic or polycyclic aliphatic ring group substituted or unsubstituted with deuterium or a naphthyl substituted or unsubstituted with deuterium; or Structural Formula A.

In another exemplary embodiment, Ar3 and Ar4, which are identical or different, may independently be a cyclopentyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

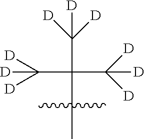 [SMILES: *C(C([2H])([2H])[2H])(C([2H])([2H])[2H])C([2H])([2H])[2H]]

or deuterium; a cyclohexyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

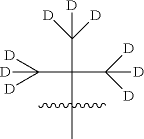 [SMILES: *C(C([2H])([2H])[2H])(C([2H])([2H])[2H])C([2H])([2H])[2H]]

or deuterium; an adamantyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

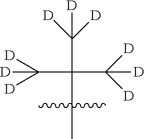 [SMILES: *C(C([2H])([2H])[2H])(C([2H])([2H])[2H])C([2H])([2H])[2H]]

or deuterium; a norbornyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

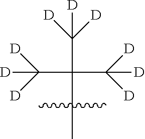 [SMILES: *C(C([2H])([2H])[2H])(C([2H])([2H])[2H])C([2H])([2H])[2H]]

or deuterium; a benzonorbornyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

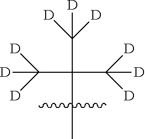 [SMILES: *C(C([2H])([2H])[2H])(C([2H])([2H])[2H])C([2H])([2H])[2H]]

or deuterium; an indanyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

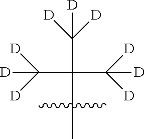 [SMILES: *C(C([2H])([2H])[2H])(C([2H])([2H])[2H])C([2H])([2H])[2H]]

or deuterium; a tetrahydronaphthyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

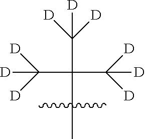 [SMILES: *C(C([2H])([2H])[2H])(C([2H])([2H])[2H])C([2H])([2H])[2H]]

or deuterium; a spiro[4.5]decanyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

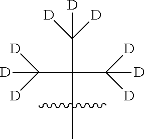 [SMILES: *C(C([2H])([2H])[2H])(C([2H])([2H])[2H])C([2H])([2H])[2H]]

or deuterium; a phenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

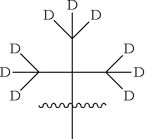 [SMILES: *C(C([2H])([2H])[2H])(C([2H])([2H])[2H])C([2H])([2H])[2H]]

or deuterium; a biphenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

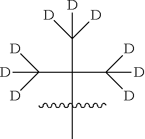 [SMILES: *C(C([2H])([2H])[2H])(C([2H])([2H])[2H])C([2H])([2H])[2H]]

or deuterium; a terphenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

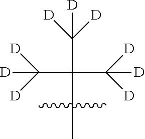 [SMILES: *C(C([2H])([2H])[2H])(C([2H])([2H])[2H])C([2H])([2H])[2H]]

or deuterium; a naphthyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

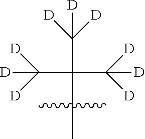 [SMILES: *C(C([2H])([2H])[2H])(C([2H])([2H])[2H])C([2H])([2H])[2H]]

or deuterium; a triphenylene substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

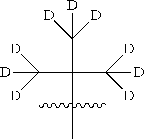 [SMILES: *C(C([2H])([2H])[2H])(C([2H])([2H])[2H])C([2H])([2H])[2H]]

or deuterium; a phenanthrene substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

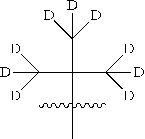 [SMILES: *C(C([2H])([2H])[2H])(C([2H])([2H])[2H])C([2H])([2H])[2H]]

or deuterium; a fluorenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

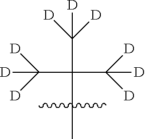 [SMILES: *C(C([2H])([2H])[2H])(C([2H])([2H])[2H])C([2H])([2H])[2H]]

or deuterium; a benzofluorenyl group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl group substituted or unsubstituted with

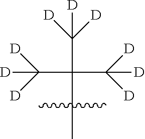 [SMILES: *C(C([2H])([2H])[2H])(C([2H])([2H])[2H])C([2H])([2H])[2H]]

or deuterium; a dibenzofuran group substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

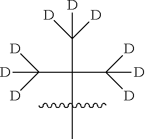 [SMILES: *C(C([2H])([2H])[2H])(C([2H])([2H])[2H])C([2H])([2H])[2H]]

or deuterium; a dibenzothiophene substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

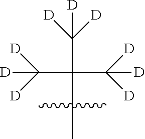 [SMILES: *C(C([2H])([2H])[2H])(C([2H])([2H])[2H])C([2H])([2H])[2H]]

or deuterium; a carbazole substituted or unsubstituted with deuterium, a methyl group substituted or unsubstituted with deuterium, an ethyl group substituted or unsubstituted with deuterium, a tert-butyl group substituted or unsubstituted with deuterium, a cyclopentyl group substituted or unsubstituted with deuterium, a cyclohexyl group substituted or unsubstituted with deuterium, a norbornyl group substituted or unsubstituted with deuterium, an adamantyl group substituted or unsubstituted with deuterium or a naphthyl substituted or unsubstituted with

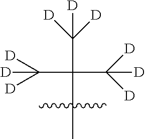 [SMILES: *C(C([2H])([2H])[2H])(C([2H])([2H])[2H])C([2H])([2H])[2H]]

or deuterium; or Structural Formula A.

In an exemplary embodiment of the present application, X may be O; S; or NR.

In another exemplary embodiment, X may be O.

In another exemplary embodiment, X may be S.

In another exemplary embodiment, X may be NR.

In another exemplary embodiment, X may be O; or S.

In another exemplary embodiment, X may be O; or NR.

In another exemplary embodiment, X may be S; or NR.

In an exemplary embodiment of the present application, R may be hydrogen; deuterium; a substituted or unsubstituted C₁ to C₄₀ alkyl group; or a substituted or unsubstituted C₆ to C₄₀ aryl group.

In another exemplary embodiment, R may be hydrogen; deuterium; a substituted or unsubstituted C₁ to C₂₀ alkyl group; or a substituted or unsubstituted C₆ to C₂₀ aryl group.

In another exemplary embodiment, R may be hydrogen; deuterium; a substituted or unsubstituted methyl group; a substituted or unsubstituted propyl group; a substituted or unsubstituted phenyl group; a substituted or unsubstituted biphenyl group; or a substituted or unsubstituted naphthyl group.

In another exemplary embodiment, R may be hydrogen; deuterium; a methyl group; a phenyl group; a biphenyl group; or a naphthyl group.

In another exemplary embodiment, R may be hydrogen or a phenyl group.

In an exemplary embodiment of the present application, X1 may be O; or S.

In another exemplary embodiment, X1 may be O.

In another exemplary embodiment, X1 may be S.

In an exemplary embodiment of the present application, Rb, Rc and Rd, which are identical or different, may independently be hydrogen; deuterium; a halogen group; a cyano group; a substituted or unsubstituted C₁ to C₄₀ alkyl group; a substituted or unsubstituted C₆ to C₄₀ aryl group; or a substituted or unsubstituted C₂ to C₄₀ heteroaryl group.

In another exemplary embodiment, Rb, Rc and Rd, which are identical or different, may independently be hydrogen; deuterium; a halogen group; a cyano group; a substituted or unsubstituted C₁ to C₂₀ alkyl group; a substituted or unsubstituted C₆ to C₂₀ aryl group; or a substituted or unsubstituted C₂ to C₂₀ heteroaryl group.

In another exemplary embodiment, Rb, Rc and Rd, which are identical or different, may independently be hydrogen; deuterium; a halogen group; a cyano group; a substituted or unsubstituted methyl group; a substituted or unsubstituted propyl group; a substituted or unsubstituted butyl group; a substituted or unsubstituted phenyl group; a substituted or unsubstituted biphenyl group; a substituted or unsubstituted naphthyl group; a substituted or unsubstituted dibenzofuran group; a substituted or unsubstituted dibenzothiophene group; or a substituted or unsubstituted carbazole group.

In another exemplary embodiment, Rb, Rc and Rd, which are identical or different, may independently be hydrogen; deuterium; a halogen group; a cyano group; a methyl group; a propyl group; a butyl group; a phenyl group; a biphenyl group; or a naphthyl group.

In another exemplary embodiment, Rb, Rc and Rd, which are identical or different, may independently be hydrogen; deuterium; or a phenyl group.

In an exemplary embodiment of the present application, rb and rd may respectively be an integer from 0 to 6, wherein if rb is 2 or greater, Rb's may be identical or different, and if rd is 2 or greater, Rd's may be identical or different.

In another exemplary embodiment, rb and rd may respectively be an integer from 0 to 5, wherein if rb is 2 or greater, Rb's may be identical or different, and if rd is 2 or greater, Rd's may be identical or different.

In another exemplary embodiment, rb and rd may respectively be an integer from 0 to 4, wherein if rb is 2 or greater, Rb's may be identical or different, and if rd is 2 or greater, Rd's may be identical or different.

In another exemplary embodiment, rb and rd may respectively be an integer from 0 to 3, wherein if rb is 2 or greater, Rb's may be identical or different, and if rd is 2 or greater, Rd's may be identical or different.

In another exemplary embodiment, rb and rd may respectively be an integer from 0 to 2, wherein if rb is 2 or greater, Rb's may be may be identical or different, and if rd is 2 or greater, Rd's may be identical or different.

In another exemplary embodiment, rb and rd may respectively be 0 or 1.

In an exemplary embodiment of the present application, rc may be an integer from 0 to 7, and if rc is 2 or greater, Rc's may be identical or different.

In another exemplary embodiment, rc may be an integer from 0 to 6, and if rc is 2 or greater, Rc's may be identical or different.

In another exemplary embodiment, rc may be an integer from 0 to 5, and if rc is 2 or greater, Rc's may be identical or different.

In another exemplary embodiment, rc may be an integer from 0 to 4, and if rc is 2 or greater, Rc's may be identical or different.

In another exemplary embodiment, rc may be an integer from 0 to 3, and if rc is 2 or greater, Rc's may be identical or different.

In another exemplary embodiment, rc may be an integer from 0 to 2, and if rc is 2, Rc's may be identical or different.

In another exemplary embodiment, rc may be 0 or 1.

In an exemplary embodiment of the present application, the compound of Chemical Formula 1 may have a deuterium substitution rate of 0% or 10% to 100%.

In another exemplary embodiment, the compound of Chemical Formula 1 may have a deuterium substitution rate of 10% to 80%.

In another exemplary embodiment, the compound of Chemical Formula 1 may have a deuterium substitution rate of 10% to 70%.

In another exemplary embodiment, the compound of Chemical Formula 1 may have a deuterium substitution rate of 10% to 60%.

In another exemplary embodiment, the compound of Chemical Formula 1 may have a deuterium substitution rate of 15% to 60%.

In another exemplary embodiment, the compound of Chemical Formula 1 may have a deuterium substitution rate of 100%.

In an exemplary embodiment of the present application, Chemical Formula 1 may be represented by any one of the following compounds.

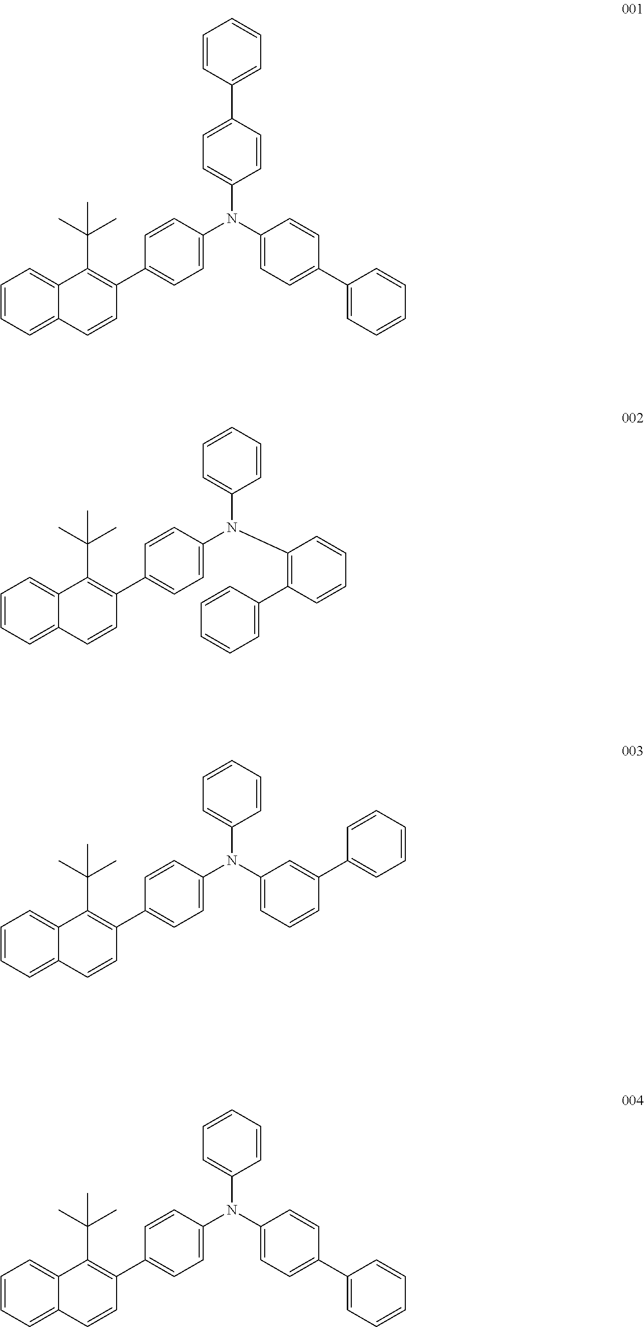 [SMILES: CC(C)(C)c1c(-c2ccc(N(c3ccc(-c4ccccc4)cc3)c3ccc(-c4ccccc4)cc3)cc2)ccc2ccccc12 ; CC(C)(C)c1c(-c2ccc(N(c3ccccc3)c3ccccc3-c3ccccc3)cc2)ccc2ccccc12 ; CC(C)(C)c1c(-c2ccc(N(c3ccccc3)c3cccc(-c4ccccc4)c3)cc2)ccc2ccccc12 ; CC(C)(C)c1c(-c2ccc(N(c3ccccc3)c3ccc(-c4ccccc4)cc3)cc2)ccc2ccccc12]

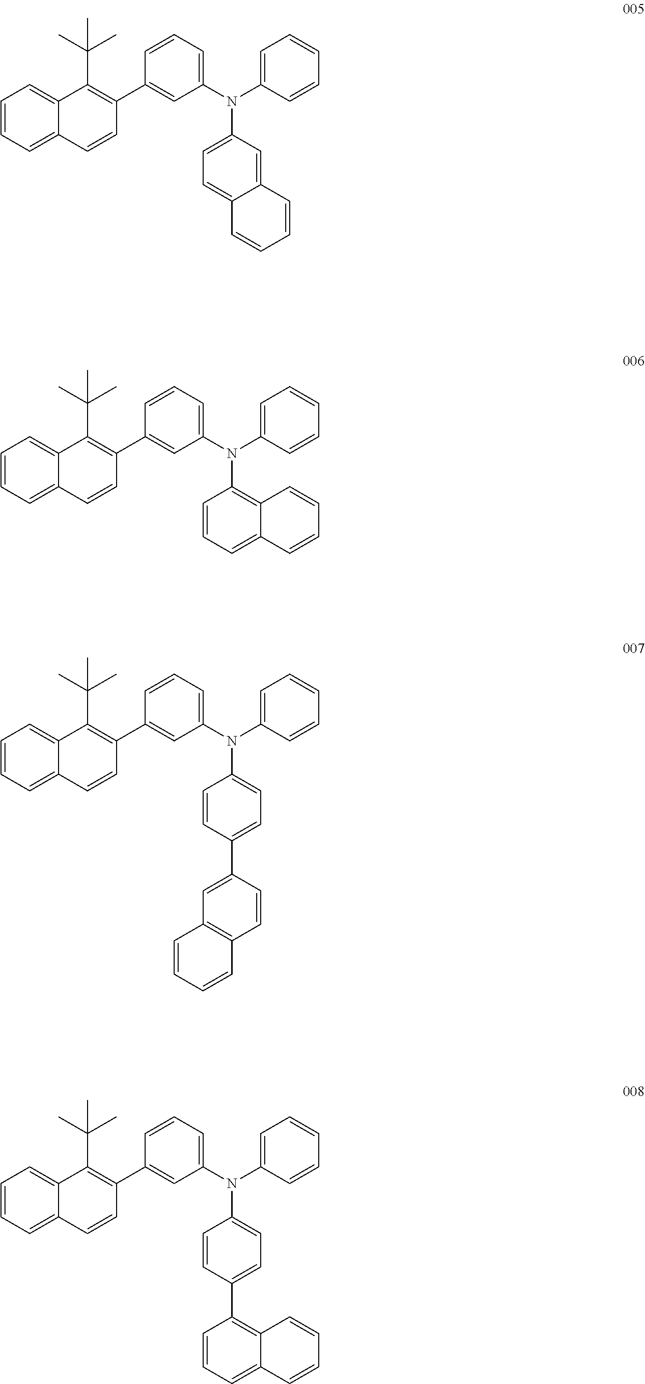 [SMILES: CC(C)(C)c1c(-c2cccc(N(c3ccccc3)c3ccc4ccccc4c3)c2)ccc2ccccc12 ; CC(C)(C)c1c(-c2cccc(N(c3ccccc3)c3cccc4ccccc34)c2)ccc2ccccc12 ; CC(C)(C)c1c(-c2cccc(N(c3ccccc3)c3ccc(-c4ccc5ccccc5c4)cc3)c2)ccc2ccccc12 ; CC(C)(C)c1c(-c2cccc(N(c3ccccc3)c3ccc(-c4cccc5ccccc45)cc3)c2)ccc2ccccc12]

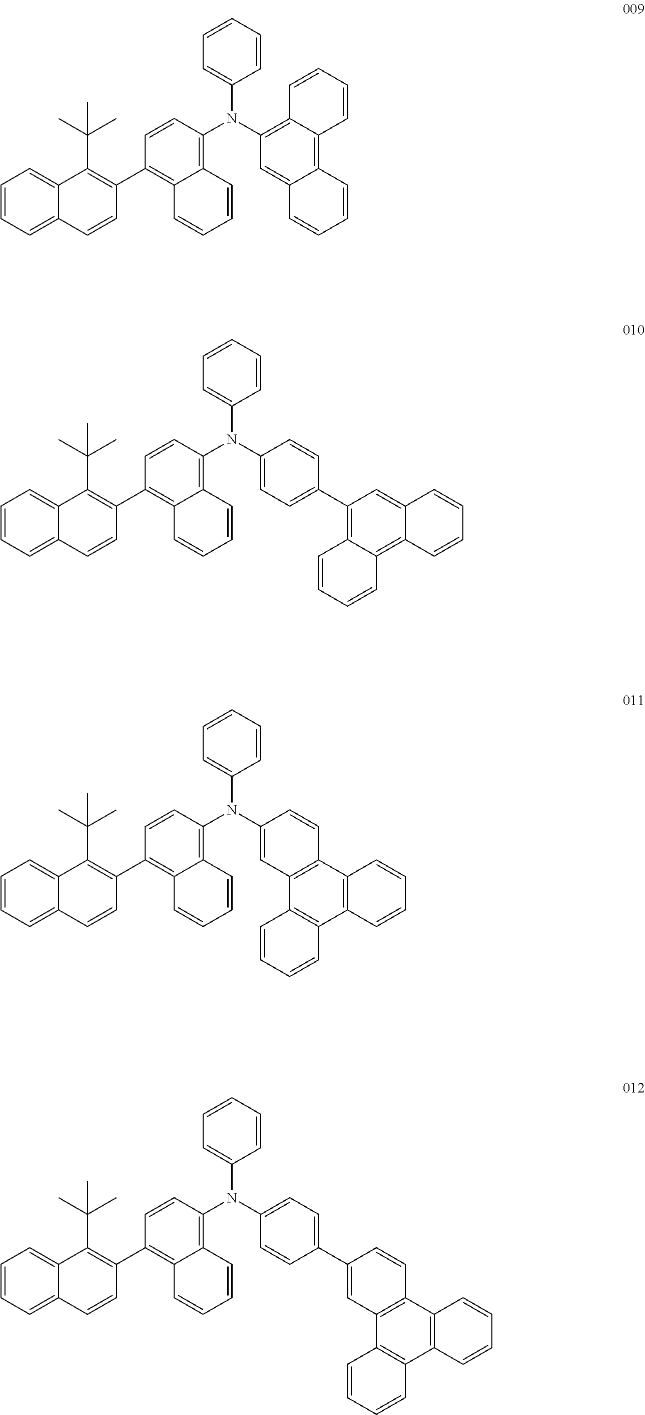 [SMILES: CC(C)(C)c1c(-c2ccc(N(c3ccccc3)c3cc4ccccc4c4ccccc34)c3ccccc23)ccc2ccccc12 ; CC(C)(C)c1c(-c2ccc(N(c3ccccc3)c3ccc(-c4cc5ccccc5c5ccccc45)cc3)c3ccccc23)ccc2ccccc12 ; CC(C)(C)c1c(-c2ccc(N(c3ccccc3)c3ccc4c5ccccc5c5ccccc5c4c3)c3ccccc23)ccc2ccccc12 ; CC(C)(C)c1c(-c2ccc(N(c3ccccc3)c3ccc(-c4ccc5c6ccccc6c6ccccc6c5c4)cc3)c3ccccc23)ccc2ccccc12]

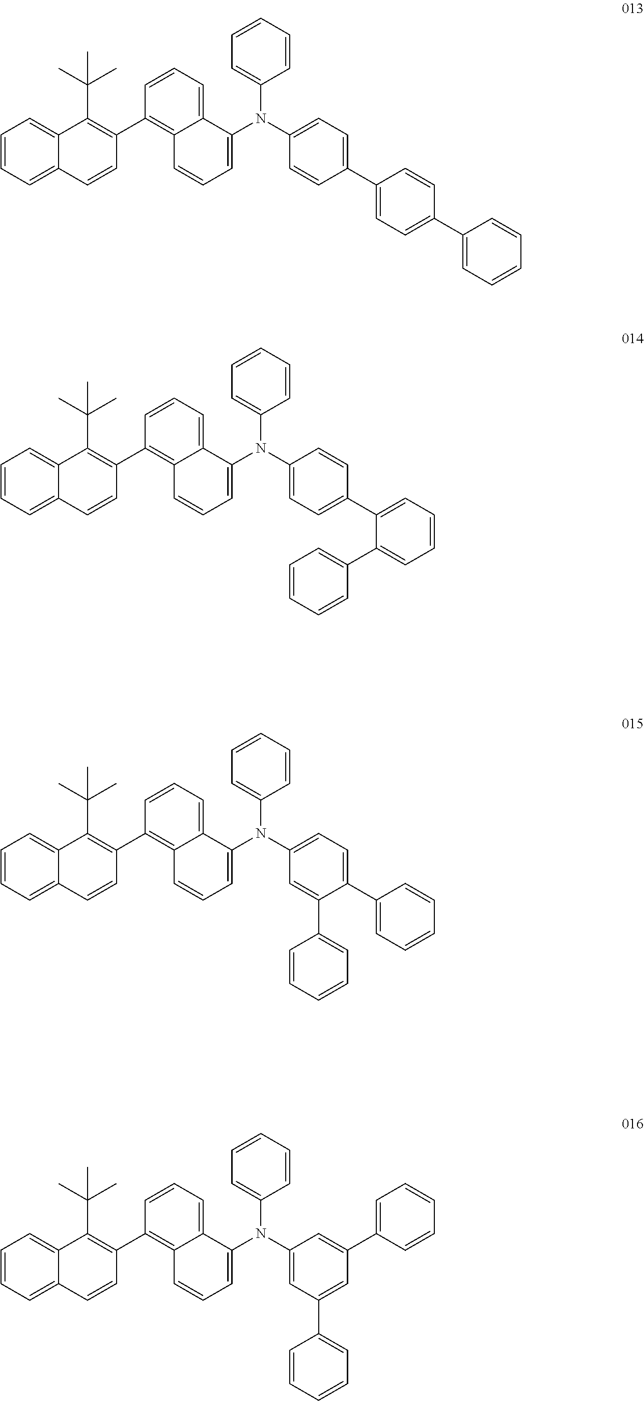 [SMILES: CC(C)(C)c1c(-c2cccc3c(N(c4ccccc4)c4ccc(-c5ccc(-c6ccccc6)cc5)cc4)cccc23)ccc2ccccc12 ; CC(C)(C)c1c(-c2cccc3c(N(c4ccccc4)c4ccc(-c5ccccc5-c5ccccc5)cc4)cccc23)ccc2ccccc12 ; CC(C)(C)c1c(-c2cccc3c(N(c4ccccc4)c4ccc(-c5ccccc5)c(-c5ccccc5)c4)cccc23)ccc2ccccc12 ; CC(C)(C)c1c(-c2cccc3c(N(c4ccccc4)c4cc(-c5ccccc5)cc(-c5ccccc5)c4)cccc23)ccc2ccccc12]

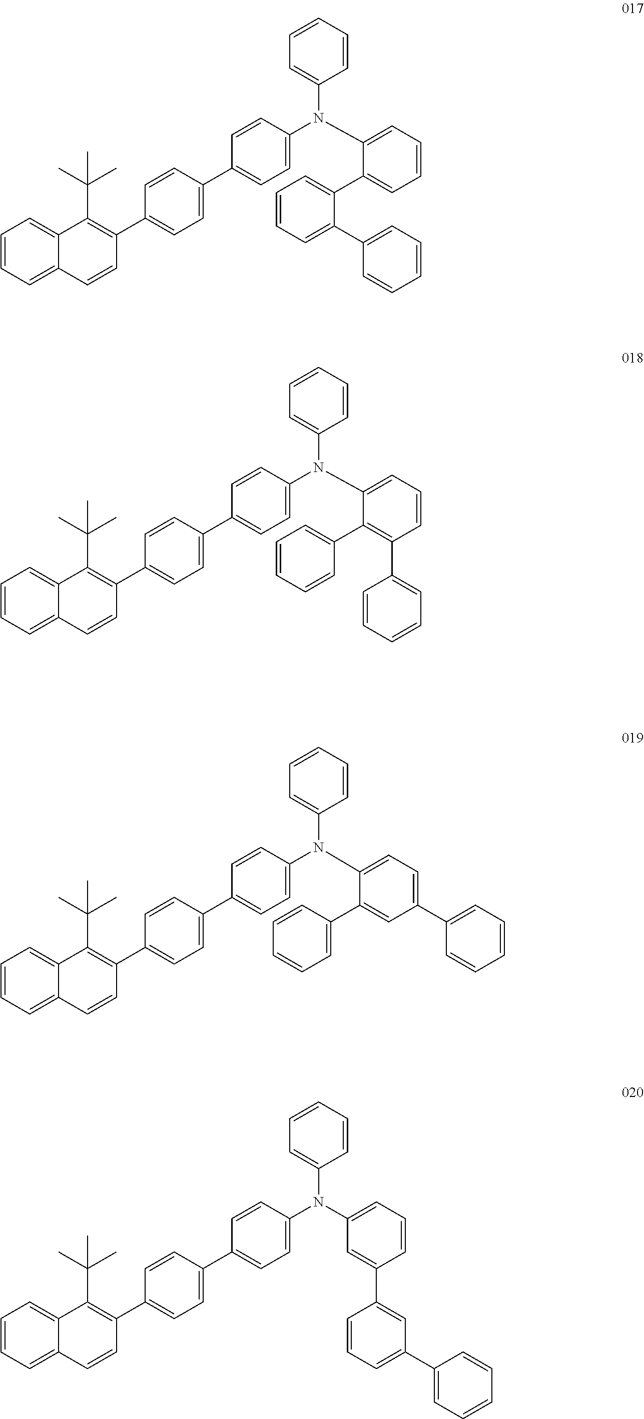 [SMILES: CC(C)(C)c1c(-c2ccc(-c3ccc(N(c4ccccc4)c4ccccc4-c4ccccc4-c4ccccc4)cc3)cc2)ccc2ccccc12 ; CC(C)(C)c1c(-c2ccc(-c3ccc(N(c4ccccc4)c4cccc(-c5ccccc5)c4-c4ccccc4)cc3)cc2)ccc2ccccc12 ; CC(C)(C)c1c(-c2ccc(-c3ccc(N(c4ccccc4)c4ccc(-c5ccccc5)cc4-c4ccccc4)cc3)cc2)ccc2ccccc12 ; CC(C)(C)c1c(-c2ccc(-c3ccc(N(c4ccccc4)c4cccc(-c5cccc(-c6ccccc6)c5)c4)cc3)cc2)ccc2ccccc12]

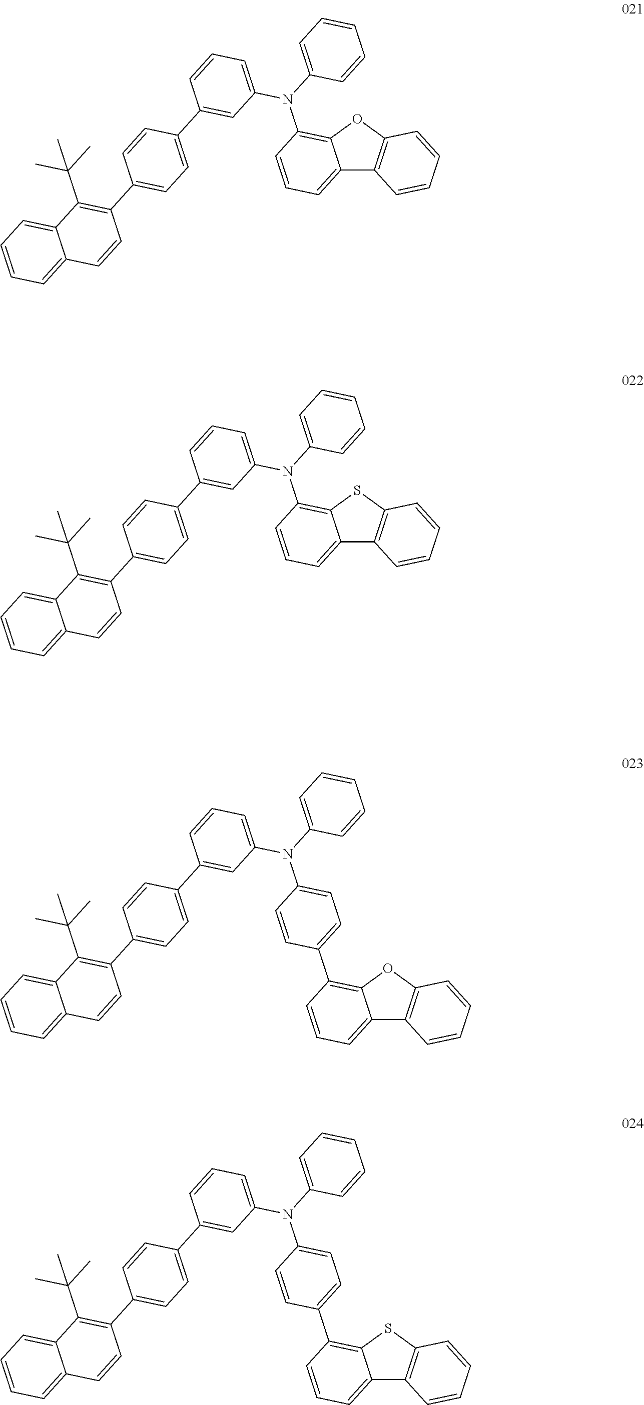 [SMILES: CC(C)(C)c1c(-c2ccc(-c3cccc(N(c4ccccc4)c4cccc5c4oc4ccccc45)c3)cc2)ccc2ccccc12 ; CC(C)(C)c1c(-c2ccc(-c3cccc(N(c4ccccc4)c4cccc5c4sc4ccccc45)c3)cc2)ccc2ccccc12 ; CC(C)(C)c1c(-c2ccc(-c3cccc(N(c4ccccc4)c4ccc(-c5cccc6c5oc5ccccc56)cc4)c3)cc2)ccc2ccccc12 ; CC(C)(C)c1c(-c2ccc(-c3cccc(N(c4ccccc4)c4ccc(-c5cccc6c5sc5ccccc56)cc4)c3)cc2)ccc2ccccc12]

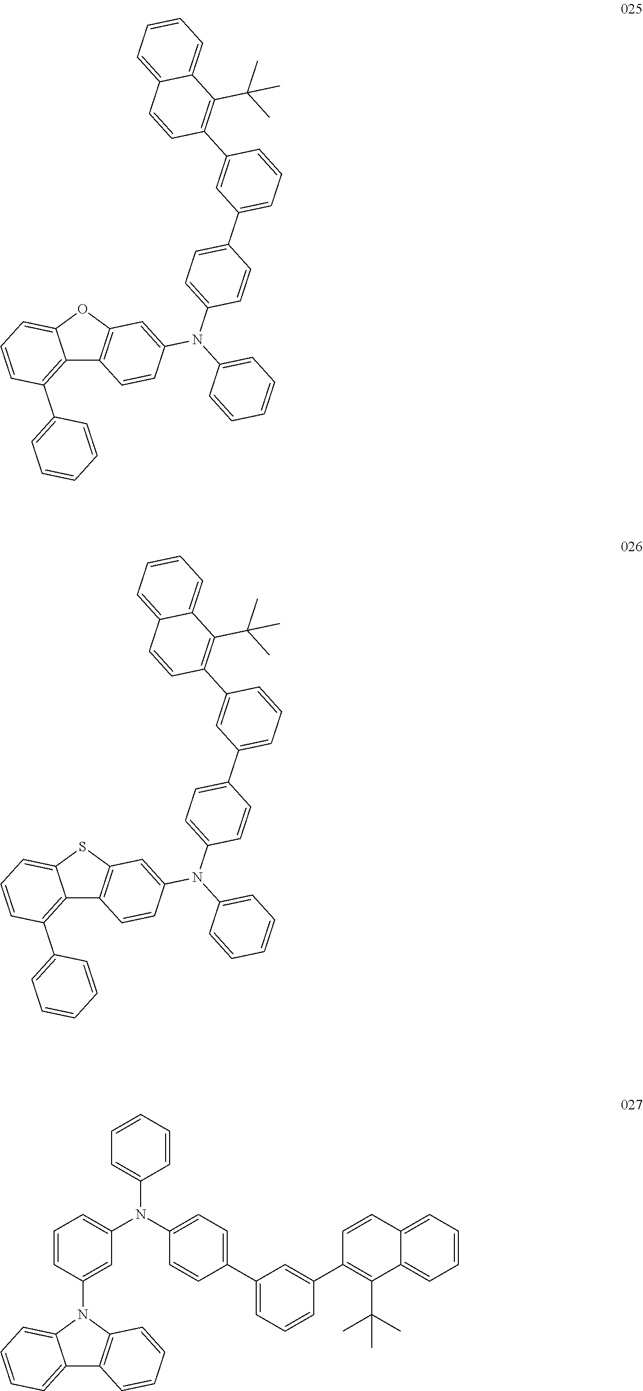 [SMILES: CC(C)(C)c1c(-c2cccc(-c3ccc(N(c4ccccc4)c4ccc5c(c4)oc4cccc(-c6ccccc6)c45)cc3)c2)ccc2ccccc12 ; CC(C)(C)c1c(-c2cccc(-c3ccc(N(c4ccccc4)c4ccc5c(c4)sc4cccc(-c6ccccc6)c45)cc3)c2)ccc2ccccc12 ; CC(C)(C)c1c(-c2cccc(-c3ccc(N(c4ccccc4)c4cccc(-n5c6ccccc6c6ccccc65)c4)cc3)c2)ccc2ccccc12]

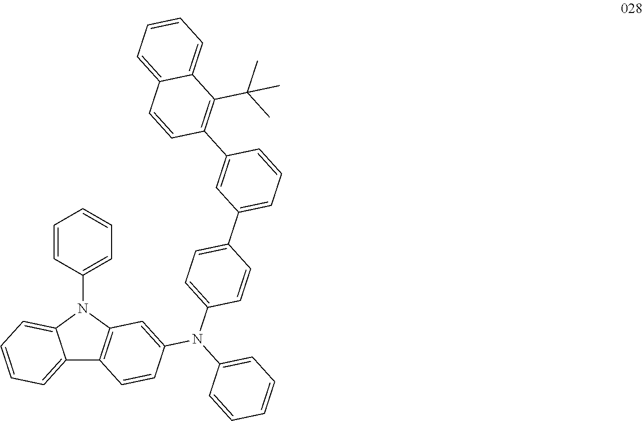 [SMILES: CC(C)(C)c1c(-c2cccc(-c3ccc(N(c4ccccc4)c4ccc5c6ccccc6n(-c6ccccc6)c5c4)cc3)c2)ccc2ccccc12]

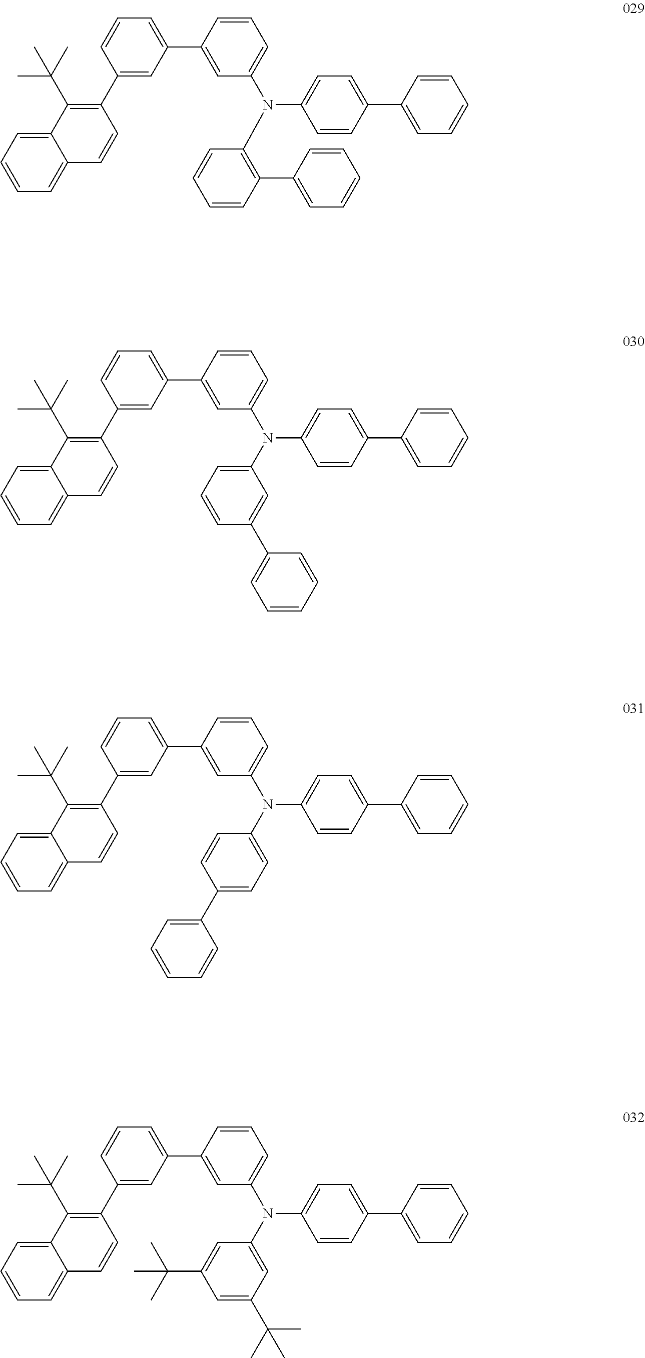 [SMILES: CC(C)(C)c1c(-c2cccc(-c3cccc(N(c4ccc(-c5ccccc5)cc4)c4ccccc4-c4ccccc4)c3)c2)ccc2ccccc12 ; CC(C)(C)c1c(-c2cccc(-c3cccc(N(c4ccc(-c5ccccc5)cc4)c4cccc(-c5ccccc5)c4)c3)c2)ccc2ccccc12 ; CC(C)(C)c1c(-c2cccc(-c3cccc(N(c4ccc(-c5ccccc5)cc4)c4ccc(-c5ccccc5)cc4)c3)c2)ccc2ccccc12 ; CC(C)(C)c1cc(N(c2ccc(-c3ccccc3)cc2)c2cccc(-c3cccc(-c4ccc5ccccc5c4C(C)(C)C)c3)c2)cc(C(C)(C)C)c1]

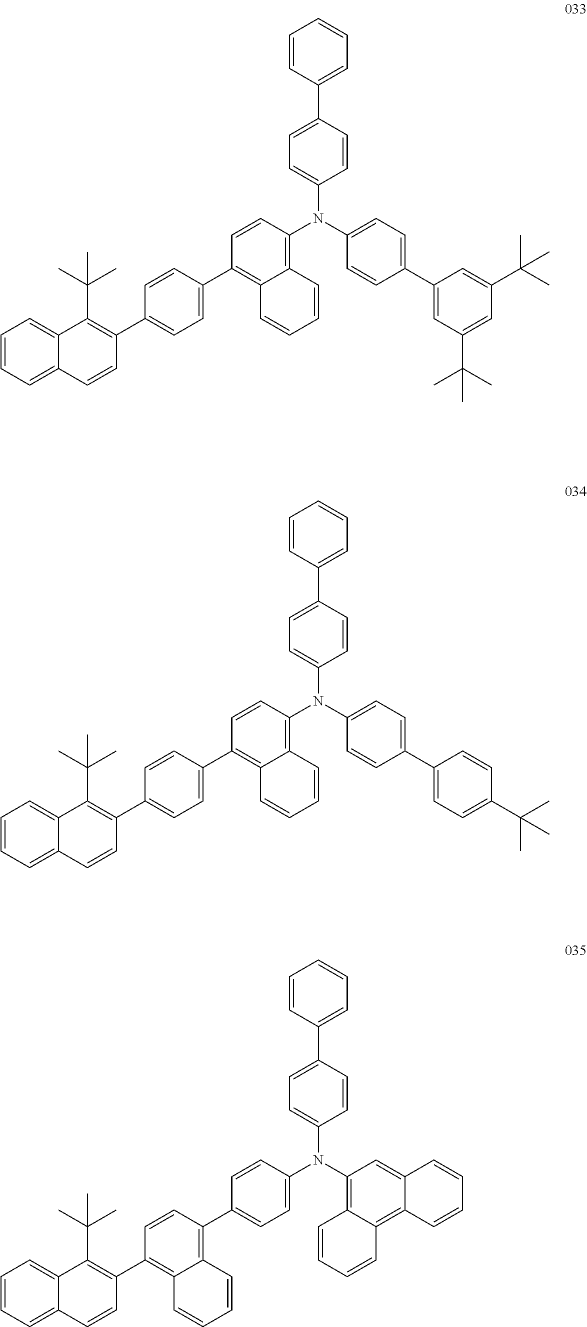 [SMILES: CC(C)(C)c1cc(-c2ccc(N(c3ccc(-c4ccccc4)cc3)c3ccc(-c4ccc(-c5ccc6ccccc6c5C(C)(C)C)cc4)c4ccccc34)cc2)cc(C(C)(C)C)c1 ; CC(C)(C)c1ccc(-c2ccc(N(c3ccc(-c4ccccc4)cc3)c3ccc(-c4ccc(-c5ccc6ccccc6c5C(C)(C)C)cc4)c4ccccc34)cc2)cc1 ; CC(C)(C)c1c(-c2ccc(-c3ccc(N(c4ccc(-c5ccccc5)cc4)c4cc5ccccc5c5ccccc45)cc3)c3ccccc23)ccc2ccccc12]

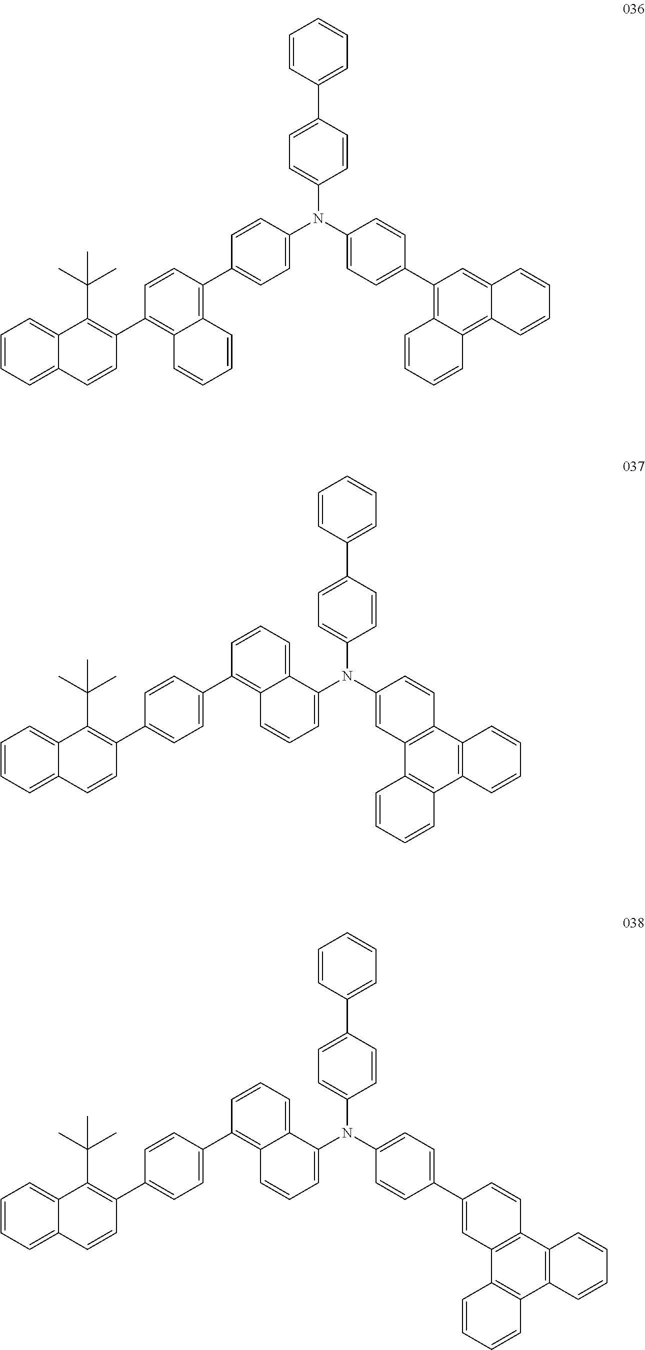 [SMILES: CC(C)(C)c1c(-c2ccc(-c3ccc(N(c4ccc(-c5ccccc5)cc4)c4ccc(-c5cc6ccccc6c6ccccc56)cc4)cc3)c3ccccc23)ccc2ccccc12 ; CC(C)(C)c1c(-c2ccc(-c3cccc4c(N(c5ccc(-c6ccccc6)cc5)c5ccc6c7ccccc7c7ccccc7c6c5)cccc34)cc2)ccc2ccccc12 ; CC(C)(C)c1c(-c2ccc(-c3cccc4c(N(c5ccc(-c6ccccc6)cc5)c5ccc(-c6ccc7c8ccccc8c8ccccc8c7c6)cc5)cccc34)cc2)ccc2ccccc12]

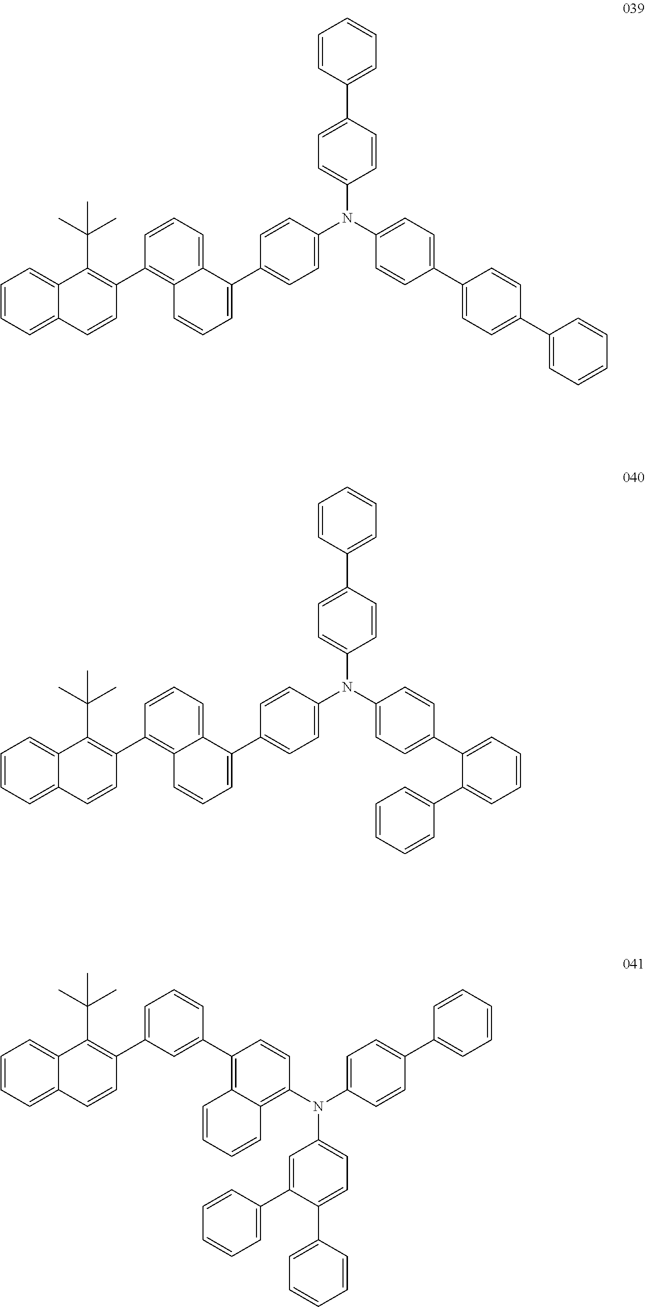 [SMILES: CC(C)(C)c1c(-c2cccc3c(-c4ccc(N(c5ccc(-c6ccccc6)cc5)c5ccc(-c6ccc(-c7ccccc7)cc6)cc5)cc4)cccc23)ccc2ccccc12 ; CC(C)(C)c1c(-c2cccc3c(-c4ccc(N(c5ccc(-c6ccccc6)cc5)c5ccc(-c6ccccc6-c6ccccc6)cc5)cc4)cccc23)ccc2ccccc12 ; CC(C)(C)c1c(-c2cccc(-c3ccc(N(c4ccc(-c5ccccc5)cc4)c4ccc(-c5ccccc5)c(-c5ccccc5)c4)c4ccccc34)c2)ccc2ccccc12]

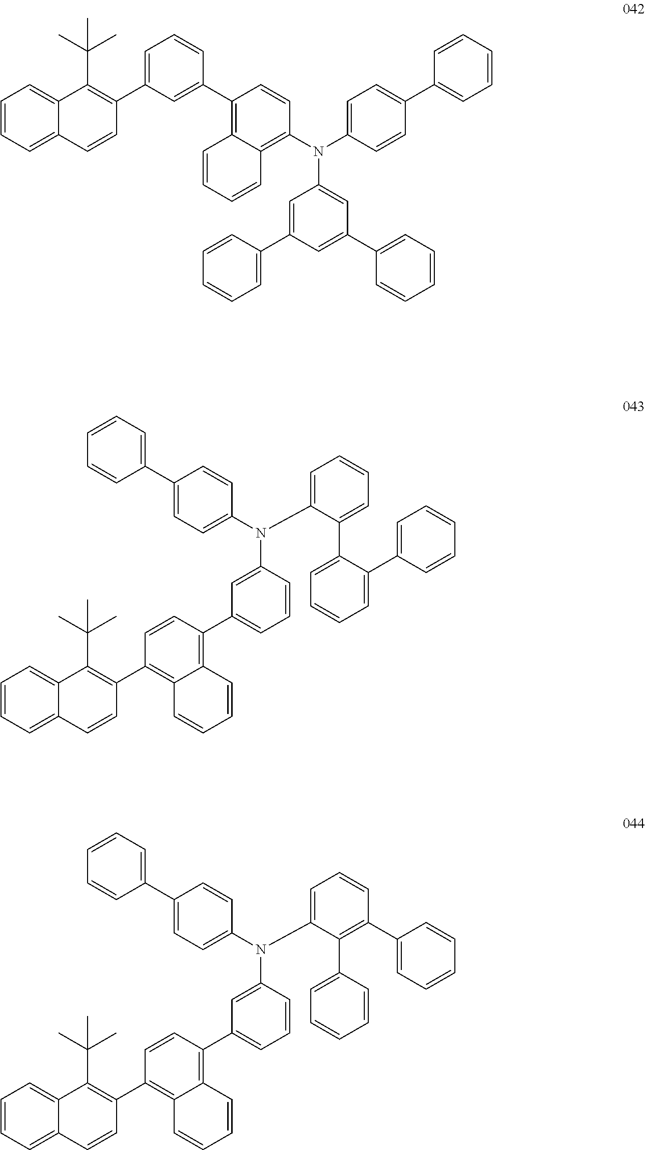 [SMILES: CC(C)(C)c1c(-c2cccc(-c3ccc(N(c4ccc(-c5ccccc5)cc4)c4cc(-c5ccccc5)cc(-c5ccccc5)c4)c4ccccc34)c2)ccc2ccccc12 ; CC(C)(C)c1c(-c2ccc(-c3cccc(N(c4ccc(-c5ccccc5)cc4)c4ccccc4-c4ccccc4-c4ccccc4)c3)c3ccccc23)ccc2ccccc12 ; CC(C)(C)c1c(-c2ccc(-c3cccc(N(c4ccc(-c5ccccc5)cc4)c4cccc(-c5ccccc5)c4-c4ccccc4)c3)c3ccccc23)ccc2ccccc12]

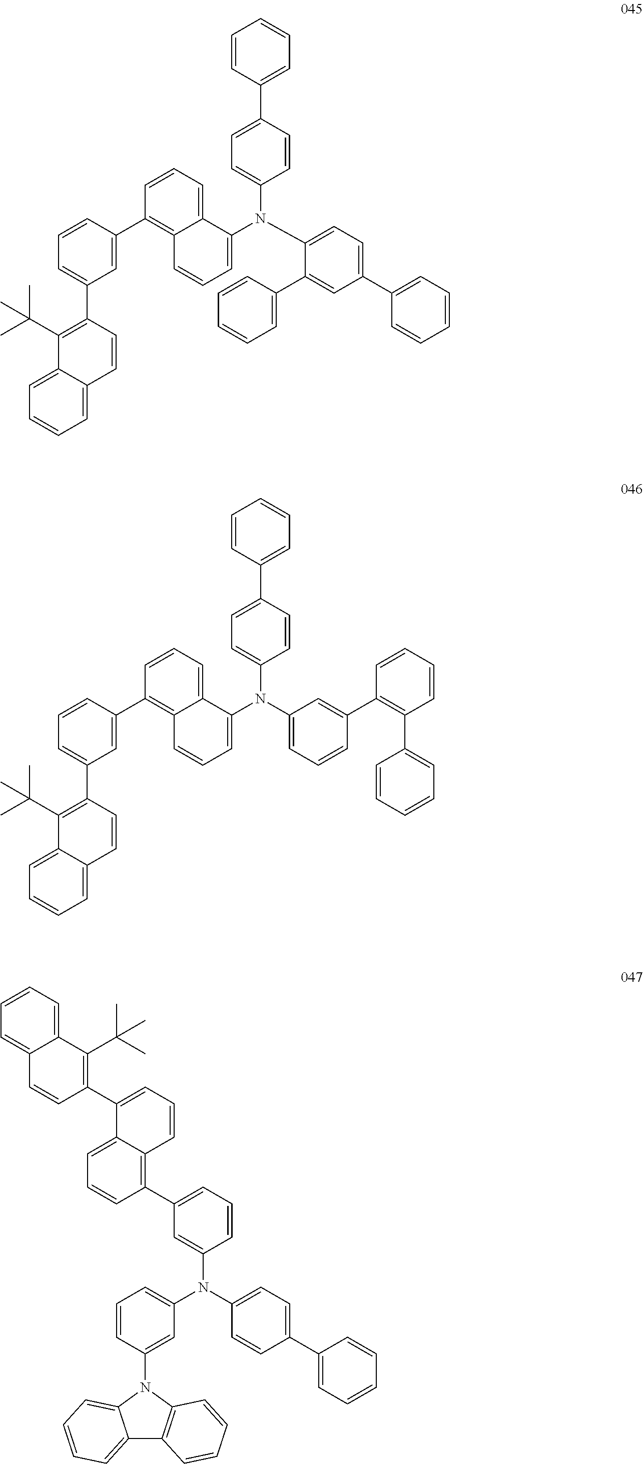 [SMILES: CC(C)(C)c1c(-c2cccc(-c3cccc4c(N(c5ccc(-c6ccccc6)cc5)c5ccc(-c6ccccc6)cc5-c5ccccc5)cccc34)c2)ccc2ccccc12 ; CC(C)(C)c1c(-c2cccc(-c3cccc4c(N(c5ccc(-c6ccccc6)cc5)c5cccc(-c6ccccc6-c6ccccc6)c5)cccc34)c2)ccc2ccccc12 ; CC(C)(C)c1c(-c2cccc3c(-c4cccc(N(c5ccc(-c6ccccc6)cc5)c5cccc(-n6c7ccccc7c7ccccc76)c5)c4)cccc23)ccc2ccccc12]

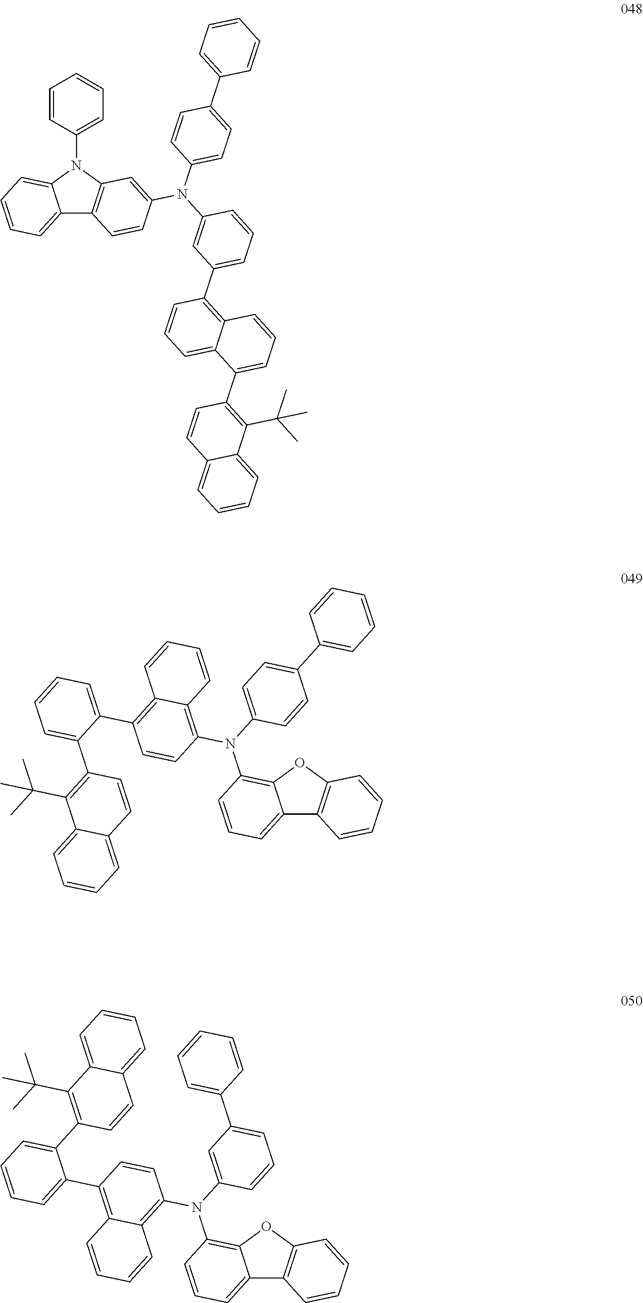

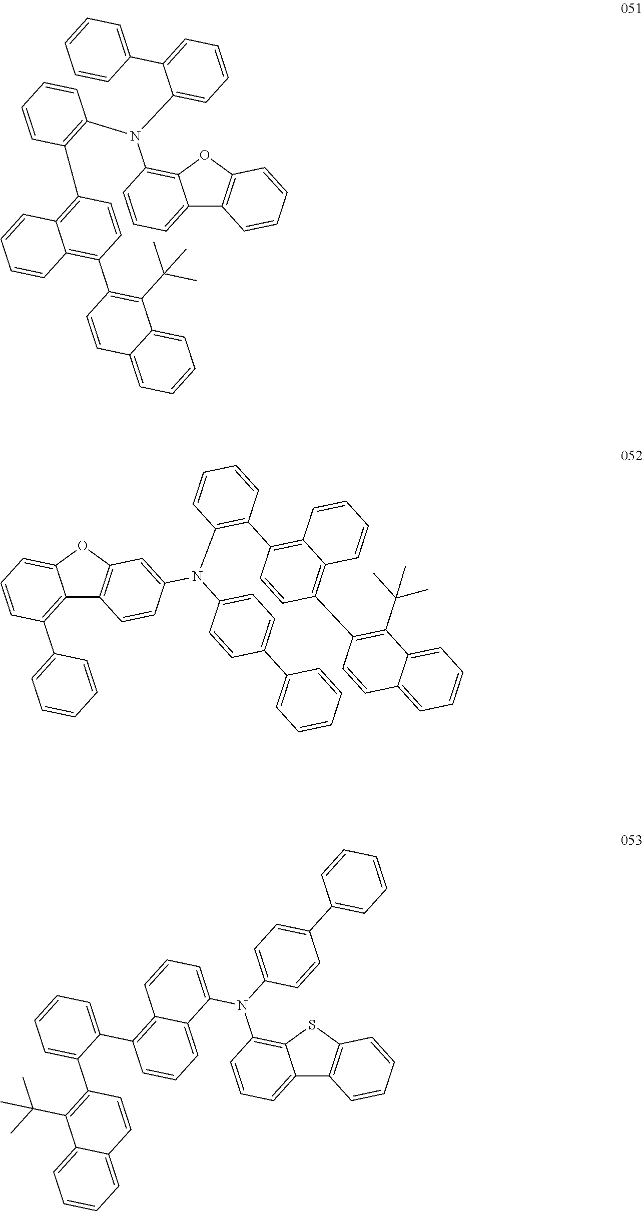 [SMILES: CC(C)(C)c1c(-c2ccc(-c3ccccc3N(c3ccccc3-c3ccccc3)c3cccc4c3oc3ccccc34)c3ccccc23)ccc2ccccc12 ; CC(C)(C)c1c(-c2ccc(-c3ccccc3N(c3ccc(-c4ccccc4)cc3)c3ccc4c(c3)oc3cccc(-c5ccccc5)c34)c3ccccc23)ccc2ccccc12 ; CC(C)(C)c1c(-c2ccccc2-c2cccc3c(N(c4ccc(-c5ccccc5)cc4)c4cccc5c4sc4ccccc45)cccc23)ccc2ccccc12]

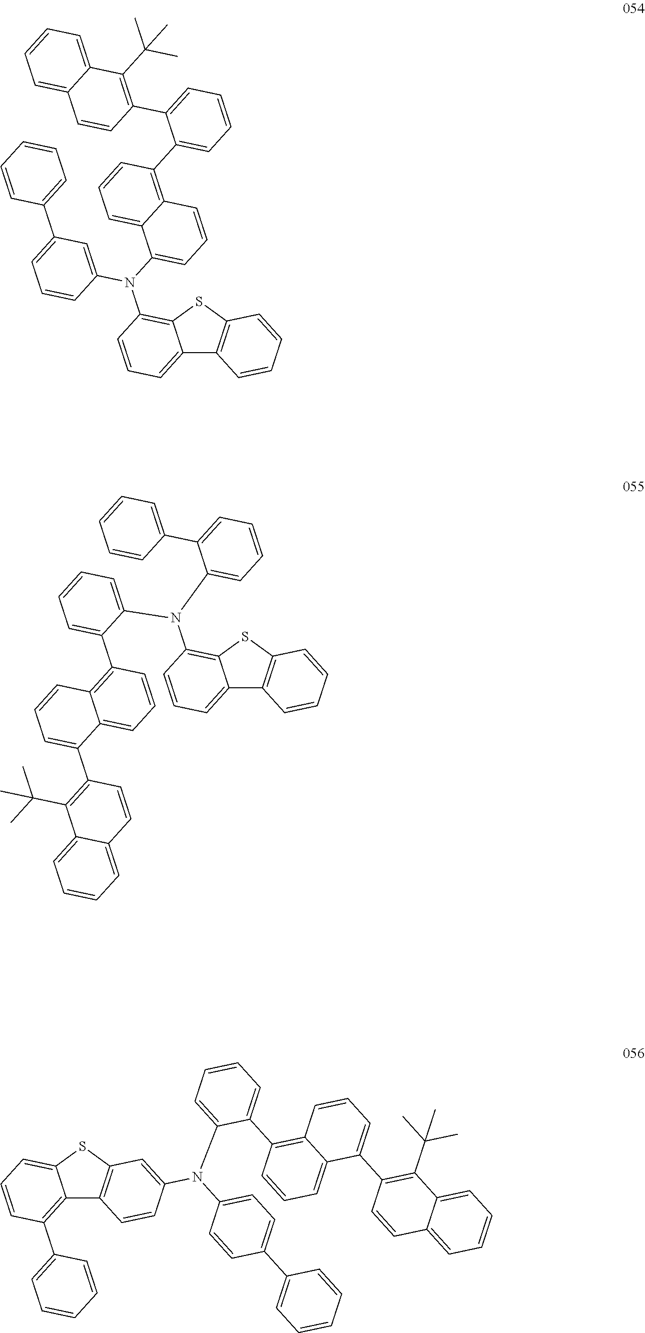 [SMILES: CC(C)(C)c1c(-c2ccccc2-c2cccc3c(N(c4cccc(-c5ccccc5)c4)c4cccc5c4sc4ccccc45)cccc23)ccc2ccccc12 ; CC(C)(C)c1c(-c2cccc3c(-c4ccccc4N(c4ccccc4-c4ccccc4)c4cccc5c4sc4ccccc45)cccc23)ccc2ccccc12 ; CC(C)(C)c1c(-c2cccc3c(-c4ccccc4N(c4ccc(-c5ccccc5)cc4)c4ccc5c(c4)sc4cccc(-c6ccccc6)c45)cccc23)ccc2ccccc12]

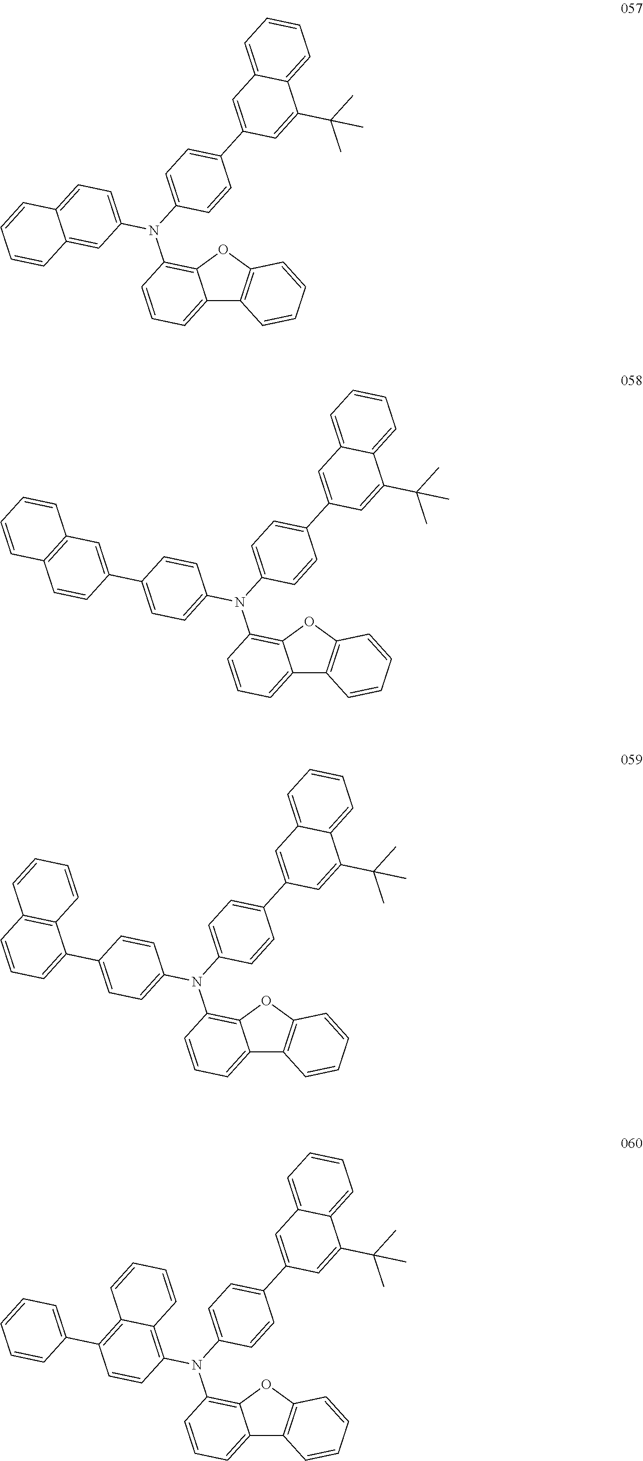 [SMILES: CC(C)(C)c1cc(-c2ccc(N(c3ccc4ccccc4c3)c3cccc4c3oc3ccccc34)cc2)cc2ccccc12 ; CC(C)(C)c1cc(-c2ccc(N(c3ccc(-c4ccc5ccccc5c4)cc3)c3cccc4c3oc3ccccc34)cc2)cc2ccccc12 ; CC(C)(C)c1cc(-c2ccc(N(c3ccc(-c4cccc5ccccc45)cc3)c3cccc4c3oc3ccccc34)cc2)cc2ccccc12 ; CC(C)(C)c1cc(-c2ccc(N(c3ccc(-c4ccccc4)c4ccccc34)c3cccc4c3oc3ccccc34)cc2)cc2ccccc12]

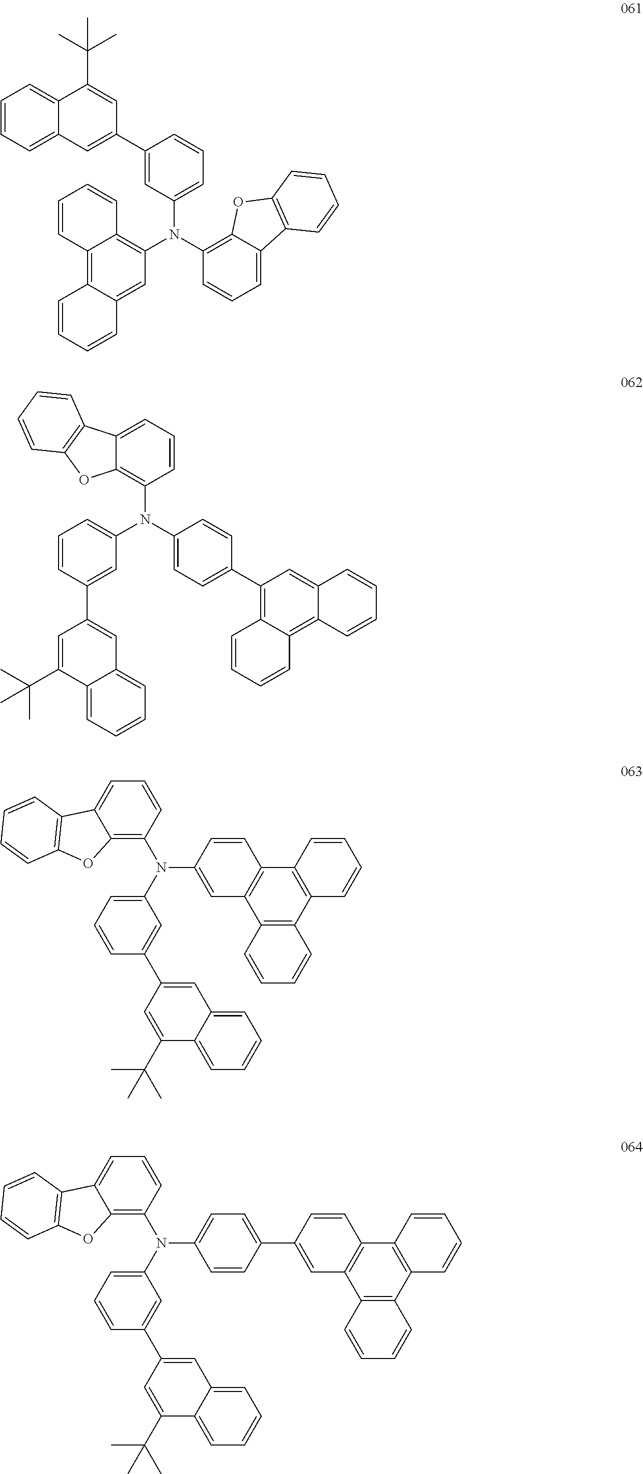

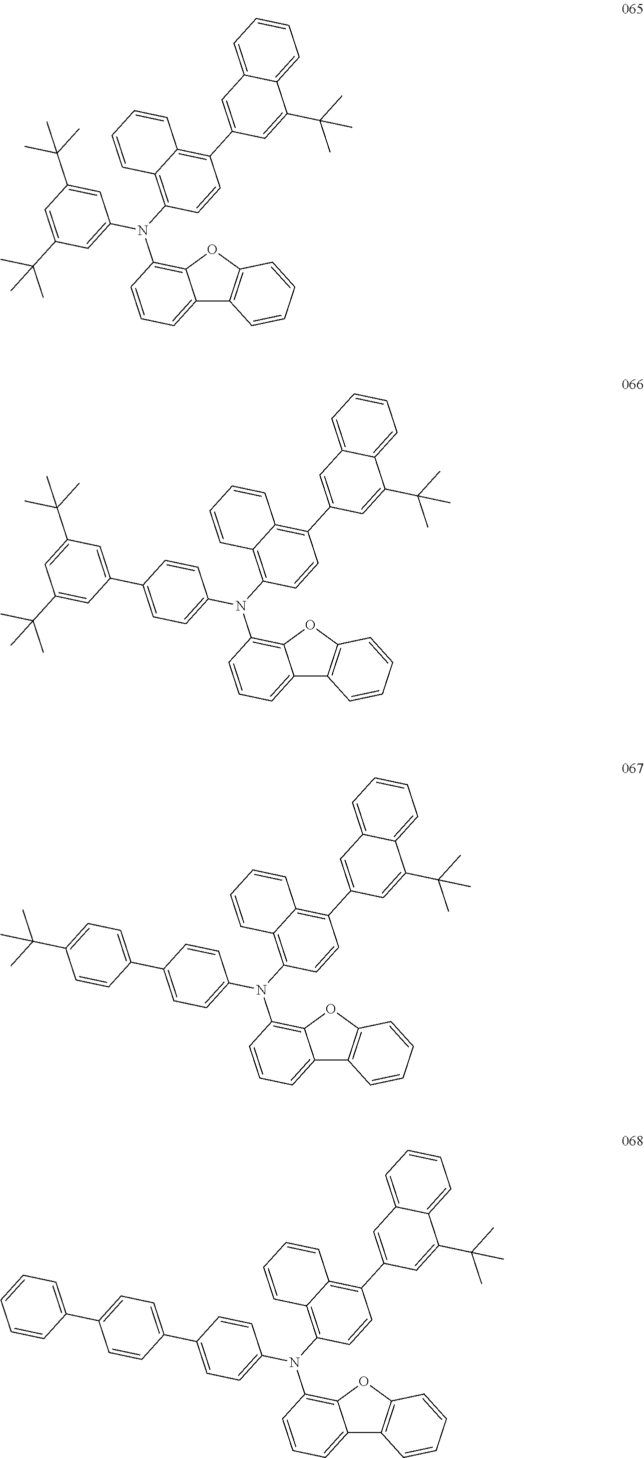

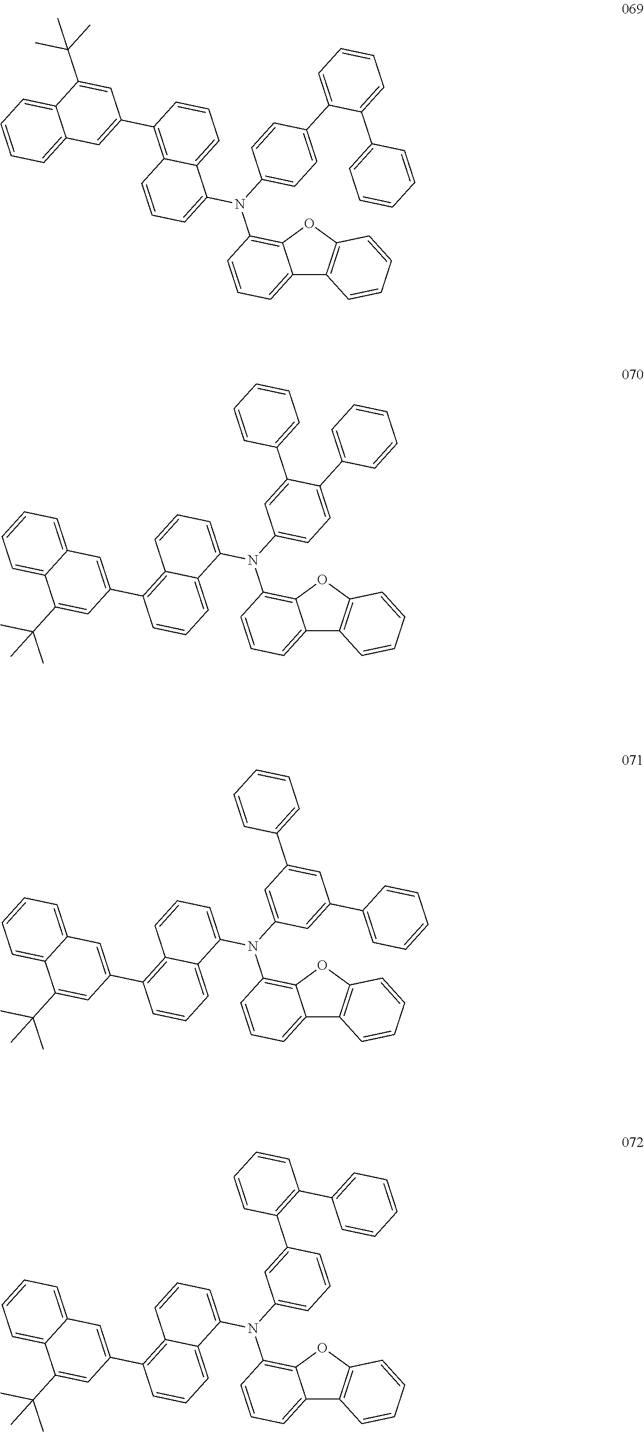 [SMILES: CC(C)(C)c1cc(-c2cccc3c(N(c4ccc(-c5ccccc5-c5ccccc5)cc4)c4cccc5c4oc4ccccc45)cccc23)cc2ccccc12 ; CC(C)(C)c1cc(-c2cccc3c(N(c4ccc(-c5ccccc5)c(-c5ccccc5)c4)c4cccc5c4oc4ccccc45)cccc23)cc2ccccc12 ; CC(C)(C)c1cc(-c2cccc3c(N(c4cc(-c5ccccc5)cc(-c5ccccc5)c4)c4cccc5c4oc4ccccc45)cccc23)cc2ccccc12 ; CC(C)(C)c1cc(-c2cccc3c(N(c4cccc(-c5ccccc5-c5ccccc5)c4)c4cccc5c4oc4ccccc45)cccc23)cc2ccccc12]

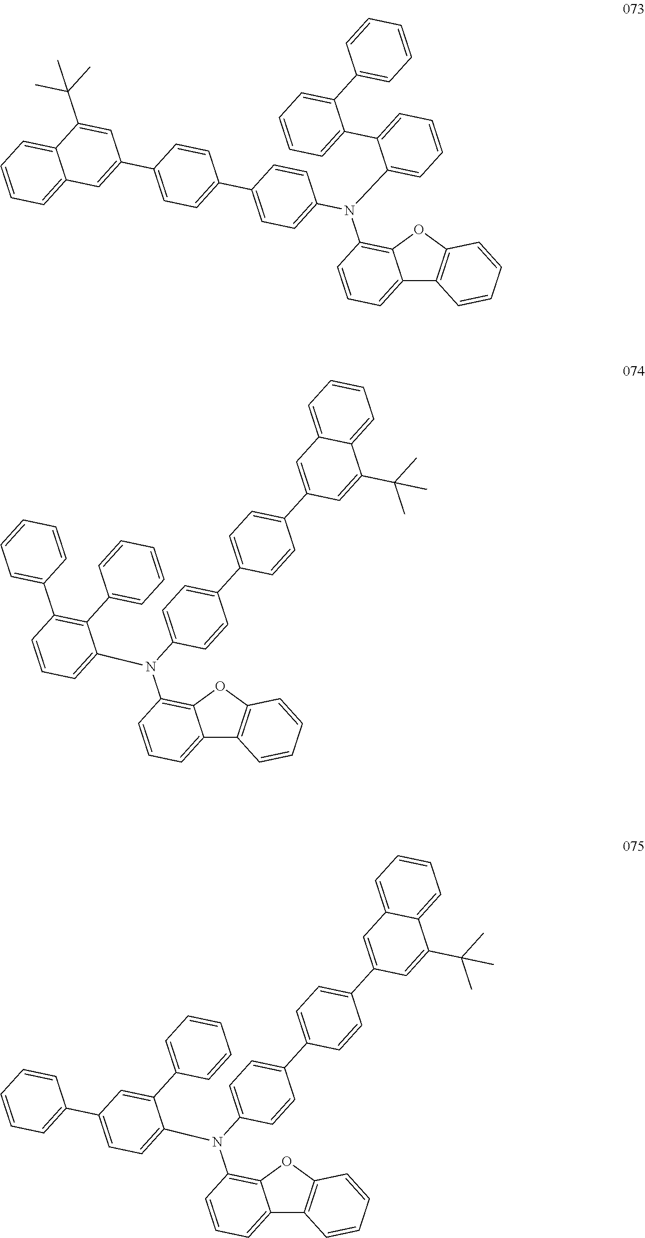 [SMILES: CC(C)(C)c1cc(-c2ccc(-c3ccc(N(c4ccccc4-c4ccccc4-c4ccccc4)c4cccc5c4oc4ccccc45)cc3)cc2)cc2ccccc12 ; CC(C)(C)c1cc(-c2ccc(-c3ccc(N(c4cccc(-c5ccccc5)c4-c4ccccc4)c4cccc5c4oc4ccccc45)cc3)cc2)cc2ccccc12 ; CC(C)(C)c1cc(-c2ccc(-c3ccc(N(c4ccc(-c5ccccc5)cc4-c4ccccc4)c4cccc5c4oc4ccccc45)cc3)cc2)cc2ccccc12]

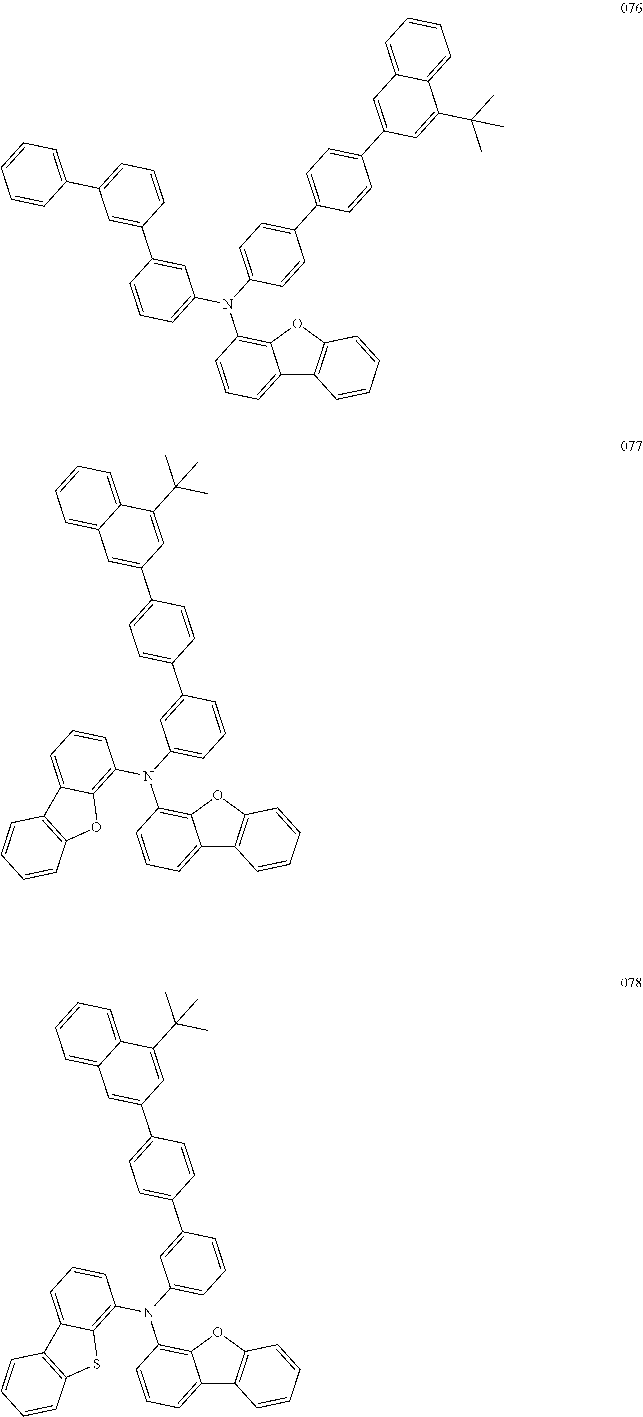 [SMILES: CC(C)(C)c1cc(-c2ccc(-c3ccc(N(c4cccc(-c5cccc(-c6ccccc6)c5)c4)c4cccc5c4oc4ccccc45)cc3)cc2)cc2ccccc12 ; CC(C)(C)c1cc(-c2ccc(-c3cccc(N(c4cccc5c4oc4ccccc45)c4cccc5c4oc4ccccc45)c3)cc2)cc2ccccc12 ; CC(C)(C)c1cc(-c2ccc(-c3cccc(N(c4cccc5c4oc4ccccc45)c4cccc5c4sc4ccccc45)c3)cc2)cc2ccccc12]

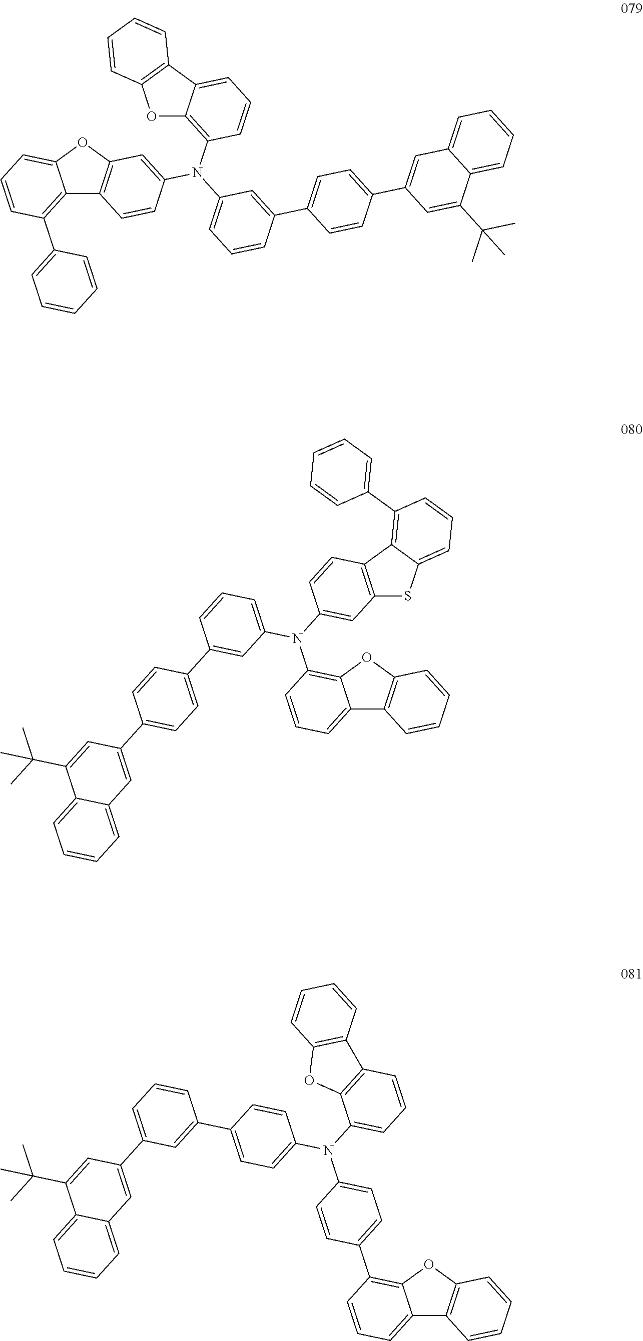

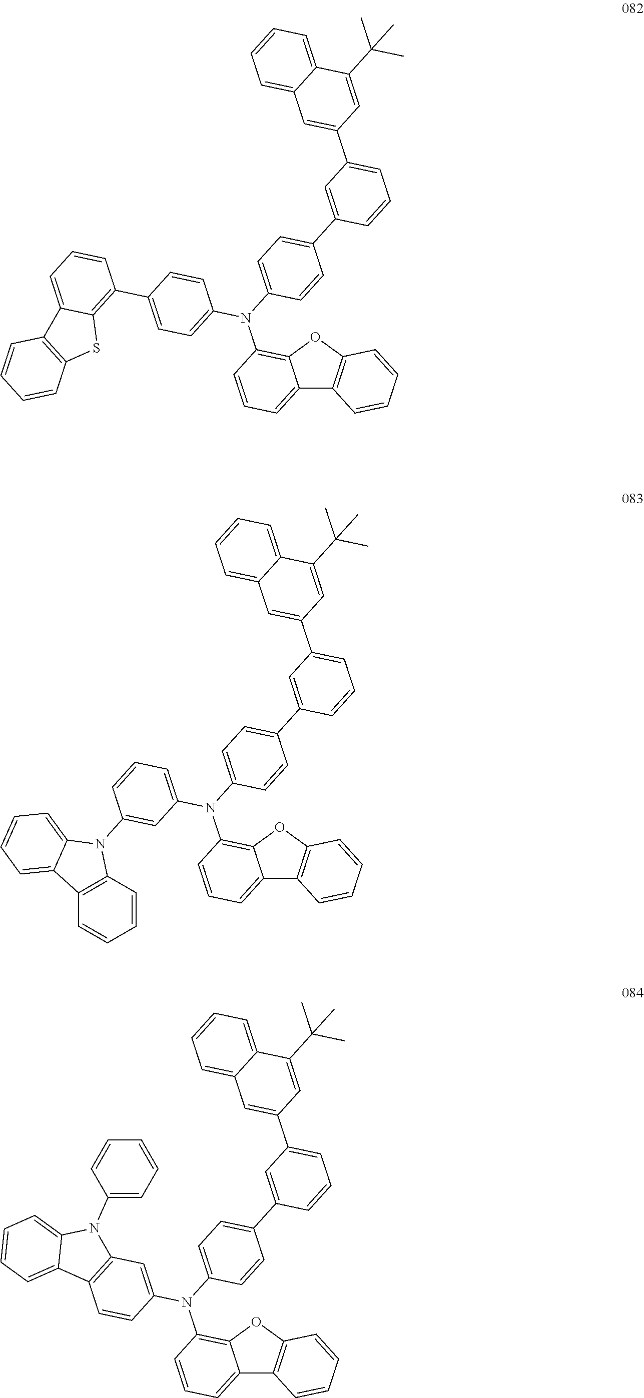 [SMILES: CC(C)(C)c1cc(-c2cccc(-c3ccc(N(c4ccc(-c5cccc6c5sc5ccccc56)cc4)c4cccc5c4oc4ccccc45)cc3)c2)cc2ccccc12 ; CC(C)(C)c1cc(-c2cccc(-c3ccc(N(c4cccc(-n5c6ccccc6c6ccccc65)c4)c4cccc5c4oc4ccccc45)cc3)c2)cc2ccccc12 ; CC(C)(C)c1cc(-c2cccc(-c3ccc(N(c4ccc5c6ccccc6n(-c6ccccc6)c5c4)c4cccc5c4oc4ccccc45)cc3)c2)cc2ccccc12]

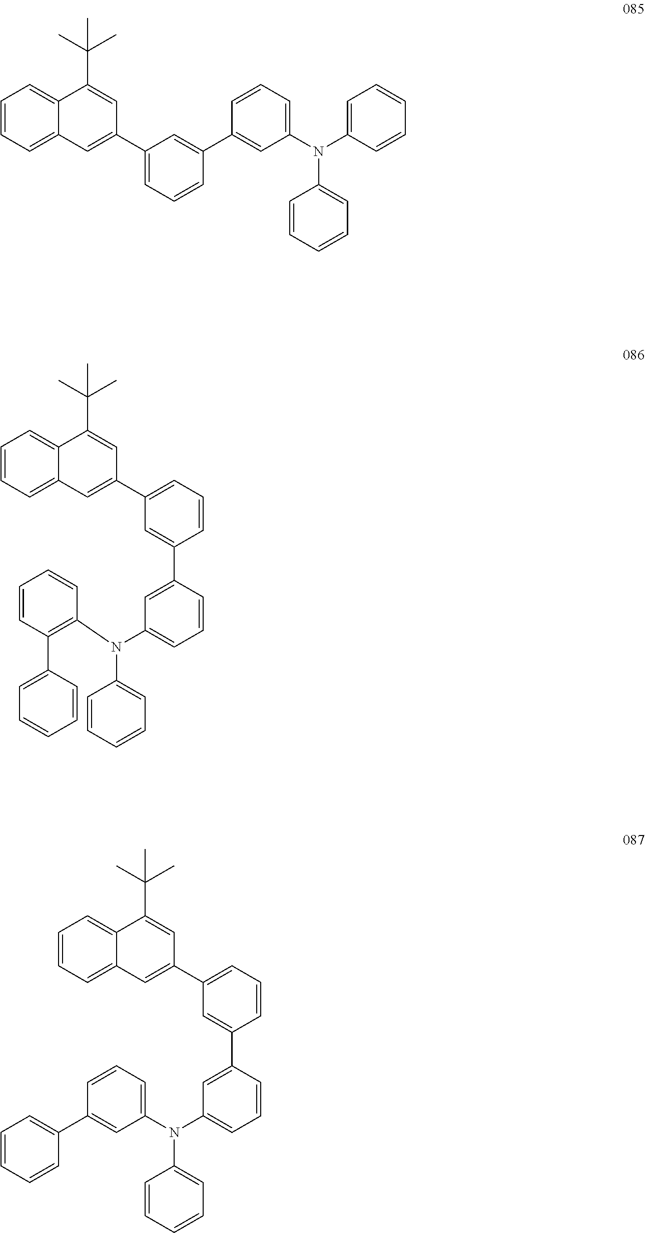 [SMILES: CC(C)(C)c1cc(-c2cccc(-c3cccc(N(c4ccccc4)c4ccccc4)c3)c2)cc2ccccc12 ; CC(C)(C)c1cc(-c2cccc(-c3cccc(N(c4ccccc4)c4ccccc4-c4ccccc4)c3)c2)cc2ccccc12 ; CC(C)(C)c1cc(-c2cccc(-c3cccc(N(c4ccccc4)c4cccc(-c5ccccc5)c4)c3)c2)cc2ccccc12]

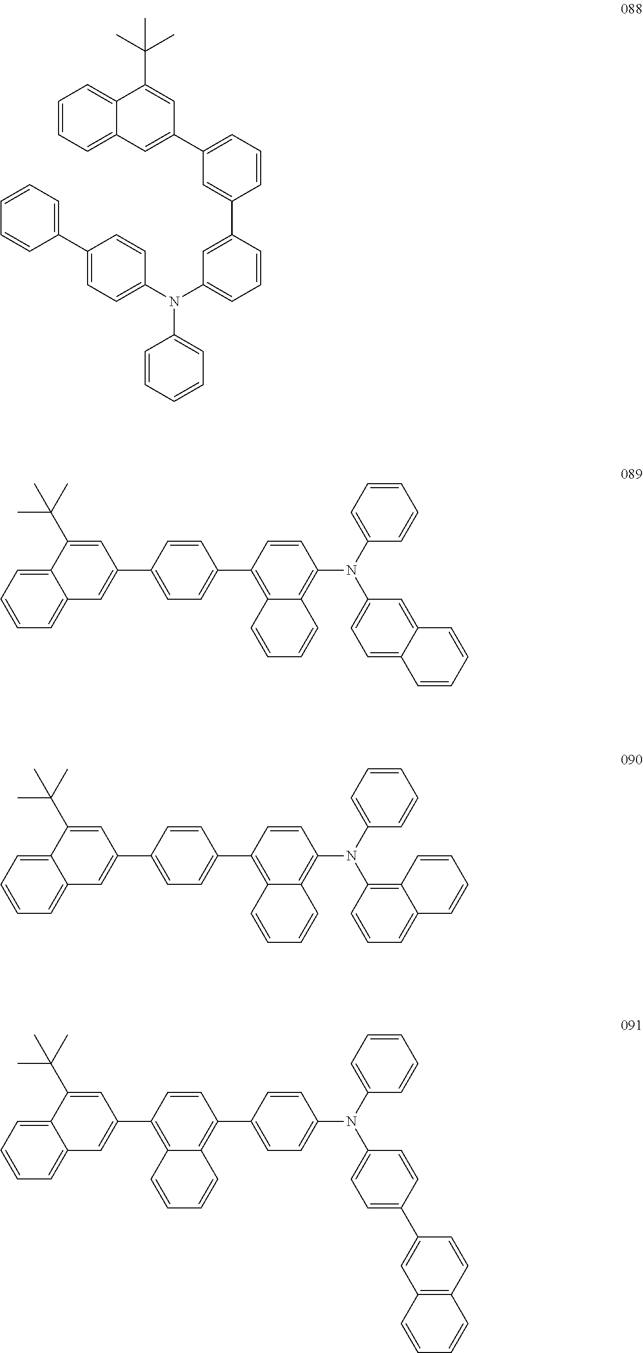 [SMILES: CC(C)(C)c1cc(-c2cccc(-c3cccc(N(c4ccccc4)c4ccc(-c5ccccc5)cc4)c3)c2)cc2ccccc12 ; CC(C)(C)c1cc(-c2ccc(-c3ccc(N(c4ccccc4)c4ccc5ccccc5c4)c4ccccc34)cc2)cc2ccccc12 ; CC(C)(C)c1cc(-c2ccc(-c3ccc(N(c4ccccc4)c4cccc5ccccc45)c4ccccc34)cc2)cc2ccccc12 ; CC(C)(C)c1cc(-c2ccc(-c3ccc(N(c4ccccc4)c4ccc(-c5ccc6ccccc6c5)cc4)cc3)c3ccccc23)cc2ccccc12]

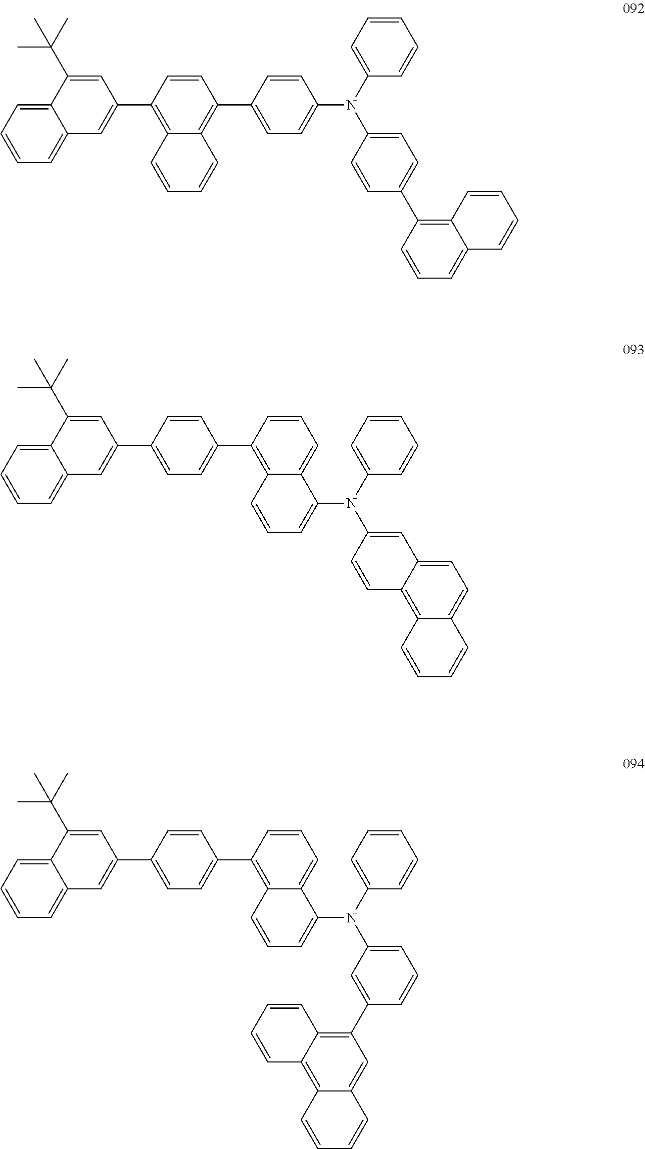 [SMILES: CC(C)(C)c1cc(-c2ccc(-c3ccc(N(c4ccccc4)c4ccc(-c5cccc6ccccc56)cc4)cc3)c3ccccc23)cc2ccccc12 ; CC(C)(C)c1cc(-c2ccc(-c3cccc4c(N(c5ccccc5)c5ccc6c(ccc7ccccc76)c5)cccc34)cc2)cc2ccccc12 ; CC(C)(C)c1cc(-c2ccc(-c3cccc4c(N(c5ccccc5)c5cccc(-c6cc7ccccc7c7ccccc67)c5)cccc34)cc2)cc2ccccc12]

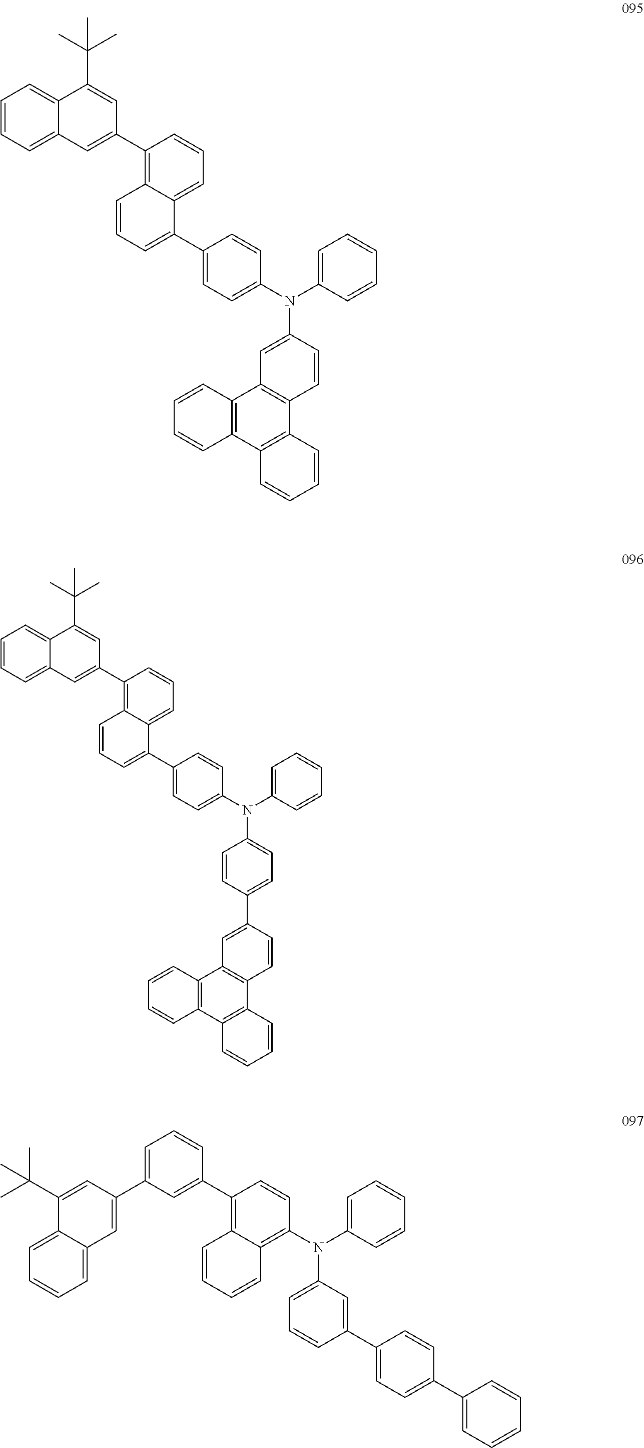 [SMILES: CC(C)(C)c1cc(-c2cccc3c(-c4ccc(N(c5ccccc5)c5ccc6c7ccccc7c7ccccc7c6c5)cc4)cccc23)cc2ccccc12 ; CC(C)(C)c1cc(-c2cccc3c(-c4ccc(N(c5ccccc5)c5ccc(-c6ccc7c8ccccc8c8ccccc8c7c6)cc5)cc4)cccc23)cc2ccccc12 ; CC(C)(C)c1cc(-c2cccc(-c3ccc(N(c4ccccc4)c4cccc(-c5ccc(-c6ccccc6)cc5)c4)c4ccccc34)c2)cc2ccccc12]

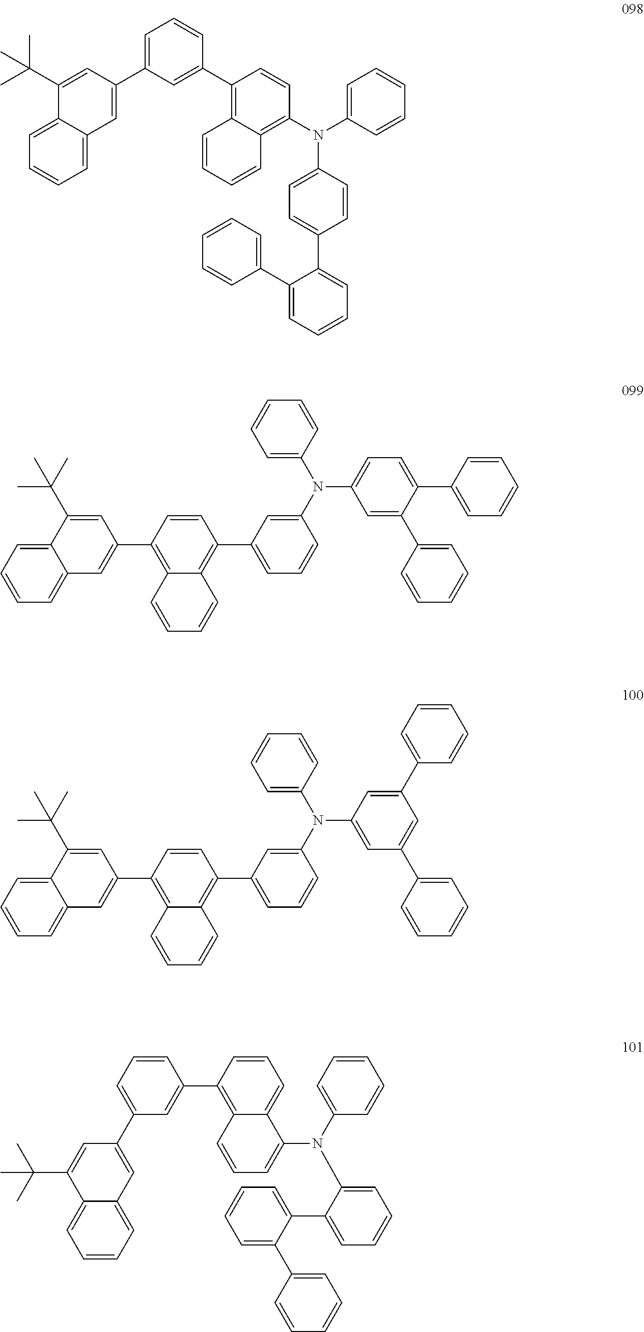 [SMILES: CC(C)(C)c1cc(-c2cccc(-c3ccc(N(c4ccccc4)c4ccc(-c5ccccc5-c5ccccc5)cc4)c4ccccc34)c2)cc2ccccc12 ; CC(C)(C)c1cc(-c2ccc(-c3cccc(N(c4ccccc4)c4ccc(-c5ccccc5)c(-c5ccccc5)c4)c3)c3ccccc23)cc2ccccc12 ; CC(C)(C)c1cc(-c2ccc(-c3cccc(N(c4ccccc4)c4cc(-c5ccccc5)cc(-c5ccccc5)c4)c3)c3ccccc23)cc2ccccc12 ; CC(C)(C)c1cc(-c2cccc(-c3cccc4c(N(c5ccccc5)c5ccccc5-c5ccccc5-c5ccccc5)cccc34)c2)cc2ccccc12]

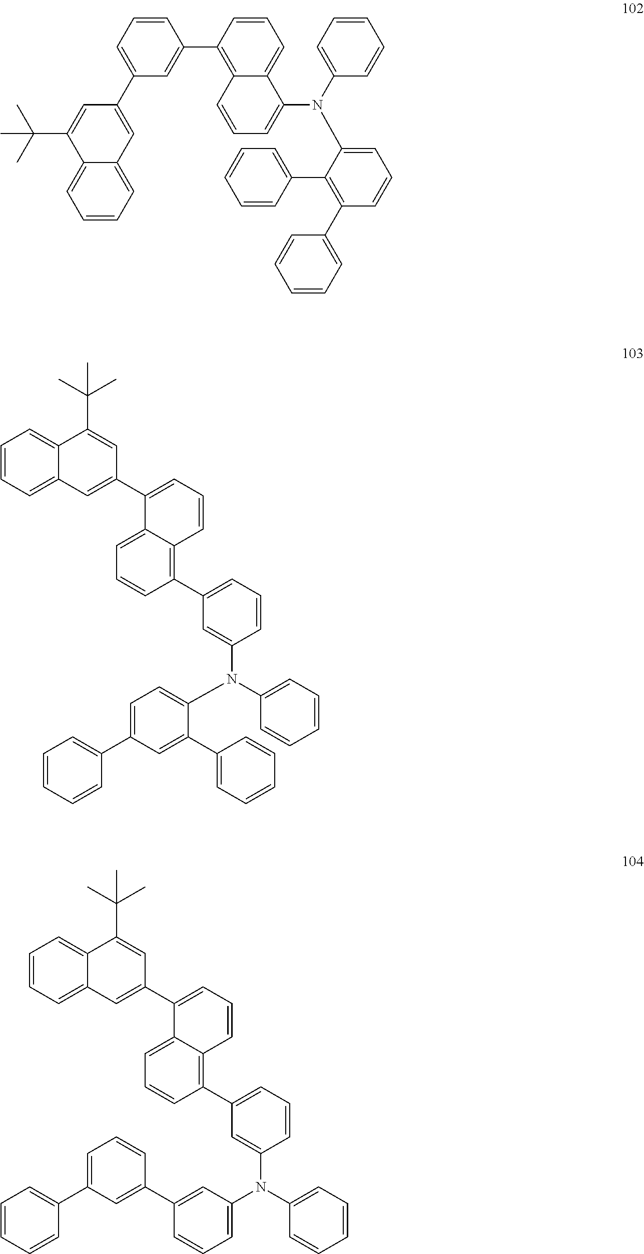 [SMILES: CC(C)(C)c1cc(-c2cccc(-c3cccc4c(N(c5ccccc5)c5cccc(-c6ccccc6)c5-c5ccccc5)cccc34)c2)cc2ccccc12 ; CC(C)(C)c1cc(-c2cccc3c(-c4cccc(N(c5ccccc5)c5ccc(-c6ccccc6)cc5-c5ccccc5)c4)cccc23)cc2ccccc12 ; CC(C)(C)c1cc(-c2cccc3c(-c4cccc(N(c5ccccc5)c5cccc(-c6cccc(-c7ccccc7)c6)c5)c4)cccc23)cc2ccccc12]

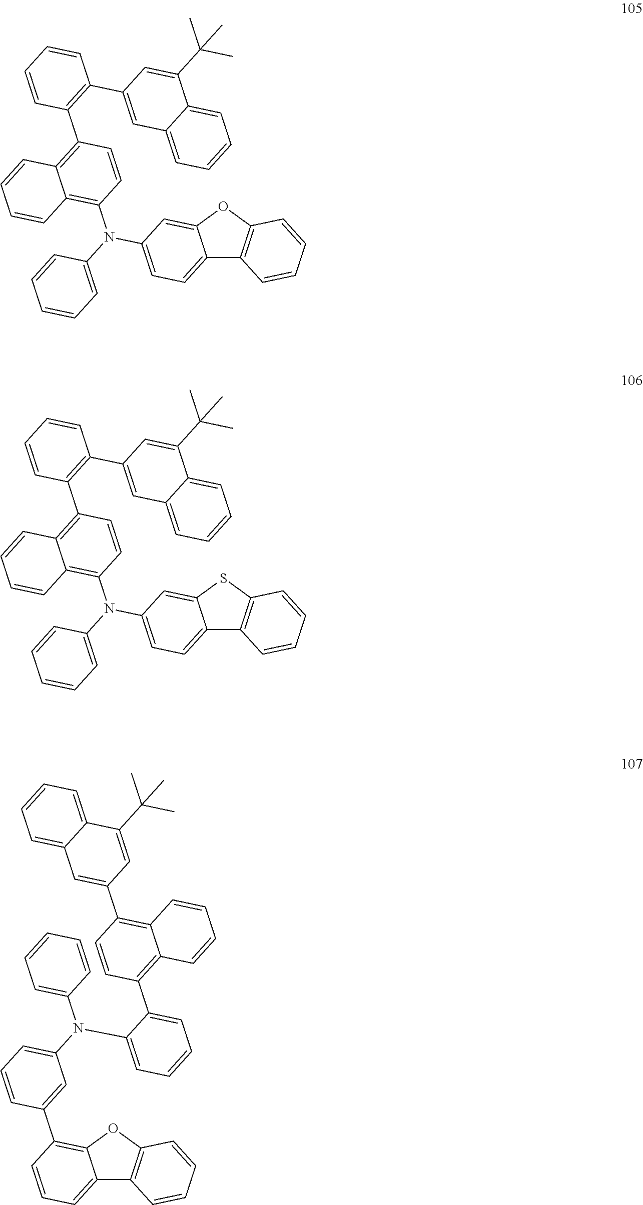 [SMILES: CC(C)(C)c1cc(-c2ccccc2-c2ccc(N(c3ccccc3)c3ccc4c(c3)oc3ccccc34)c3ccccc23)cc2ccccc12 ; CC(C)(C)c1cc(-c2ccccc2-c2ccc(N(c3ccccc3)c3ccc4c(c3)sc3ccccc34)c3ccccc23)cc2ccccc12 ; CC(C)(C)c1cc(-c2ccc(-c3ccccc3N(c3ccccc3)c3cccc(-c4cccc5c4oc4ccccc45)c3)c3ccccc23)cc2ccccc12]

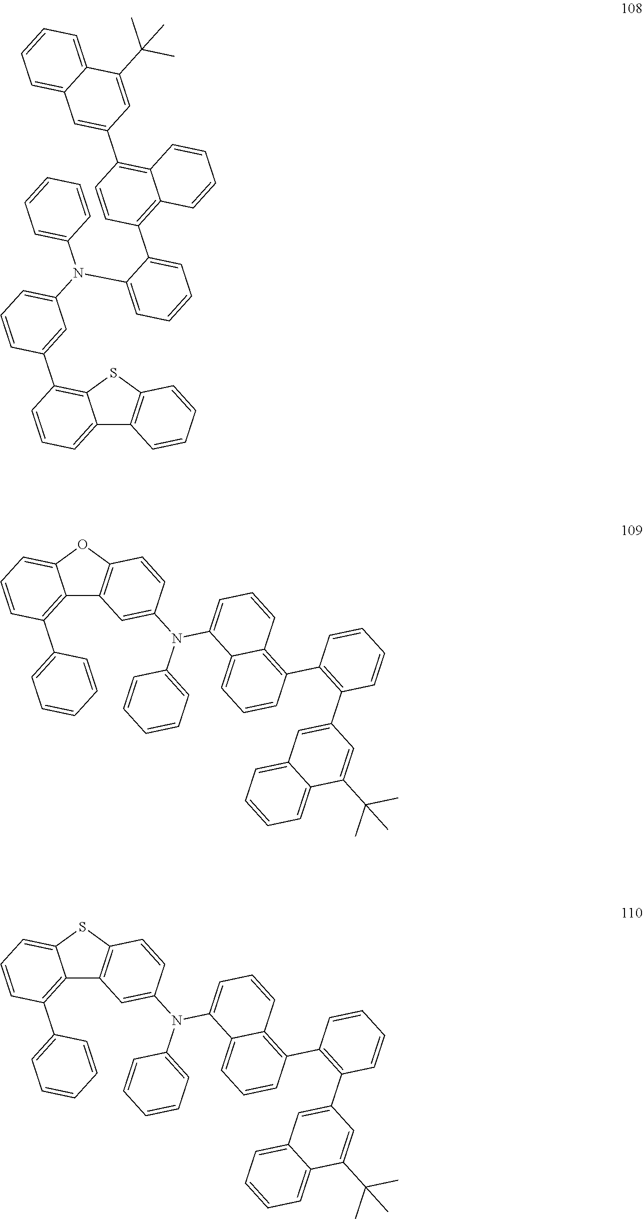 [SMILES: CC(C)(C)c1cc(-c2ccc(-c3ccccc3N(c3ccccc3)c3cccc(-c4cccc5c4sc4ccccc45)c3)c3ccccc23)cc2ccccc12 ; CC(C)(C)c1cc(-c2ccccc2-c2cccc3c(N(c4ccccc4)c4ccc5oc6cccc(-c7ccccc7)c6c5c4)cccc23)cc2ccccc12 ; CC(C)(C)c1cc(-c2ccccc2-c2cccc3c(N(c4ccccc4)c4ccc5sc6cccc(-c7ccccc7)c6c5c4)cccc23)cc2ccccc12]

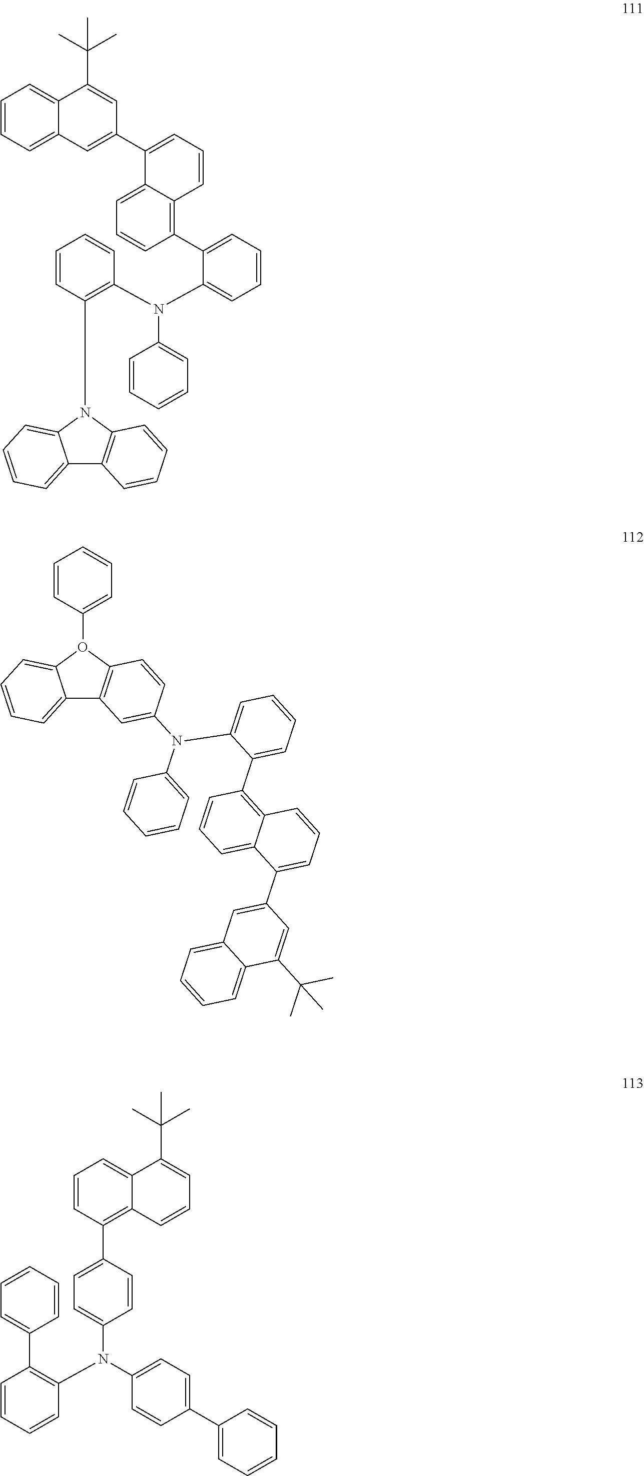

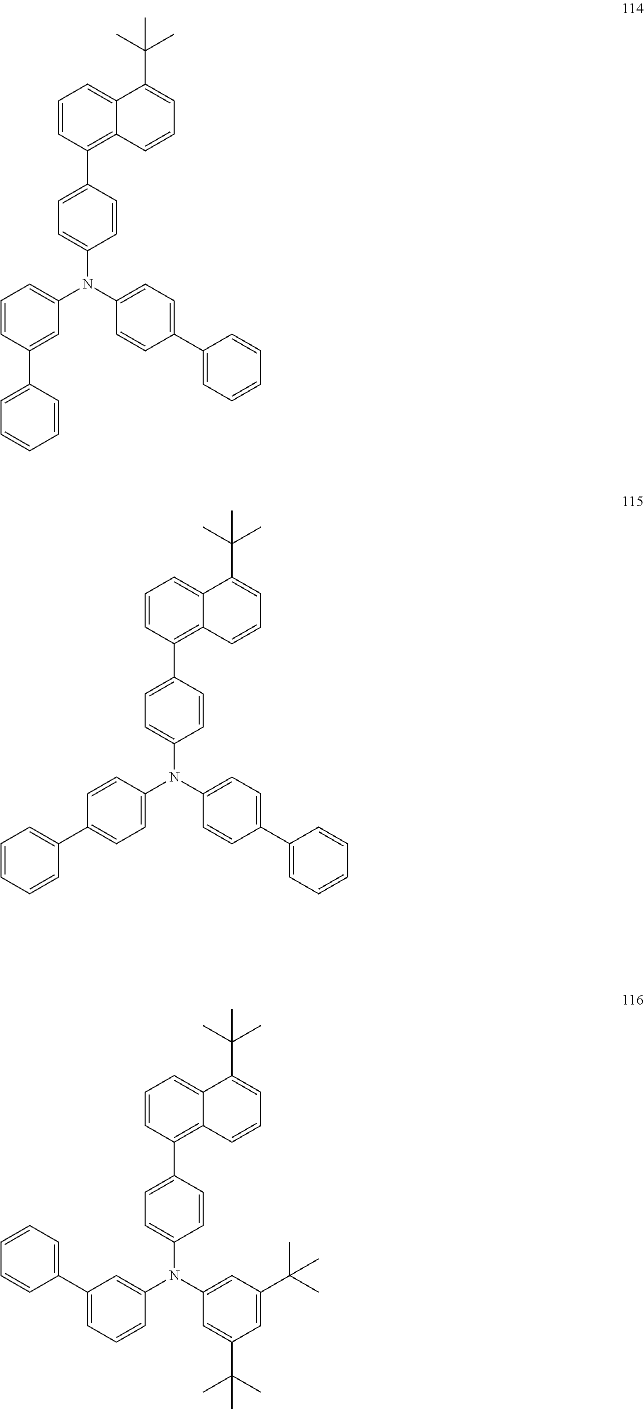 [SMILES: CC(C)(C)c1cccc2c(-c3ccc(N(c4ccc(-c5ccccc5)cc4)c4cccc(-c5ccccc5)c4)cc3)cccc12 ; CC(C)(C)c1cccc2c(-c3ccc(N(c4ccc(-c5ccccc5)cc4)c4ccc(-c5ccccc5)cc4)cc3)cccc12 ; CC(C)(C)c1cc(N(c2ccc(-c3cccc4c(C(C)(C)C)cccc34)cc2)c2cccc(-c3ccccc3)c2)cc(C(C)(C)C)c1]

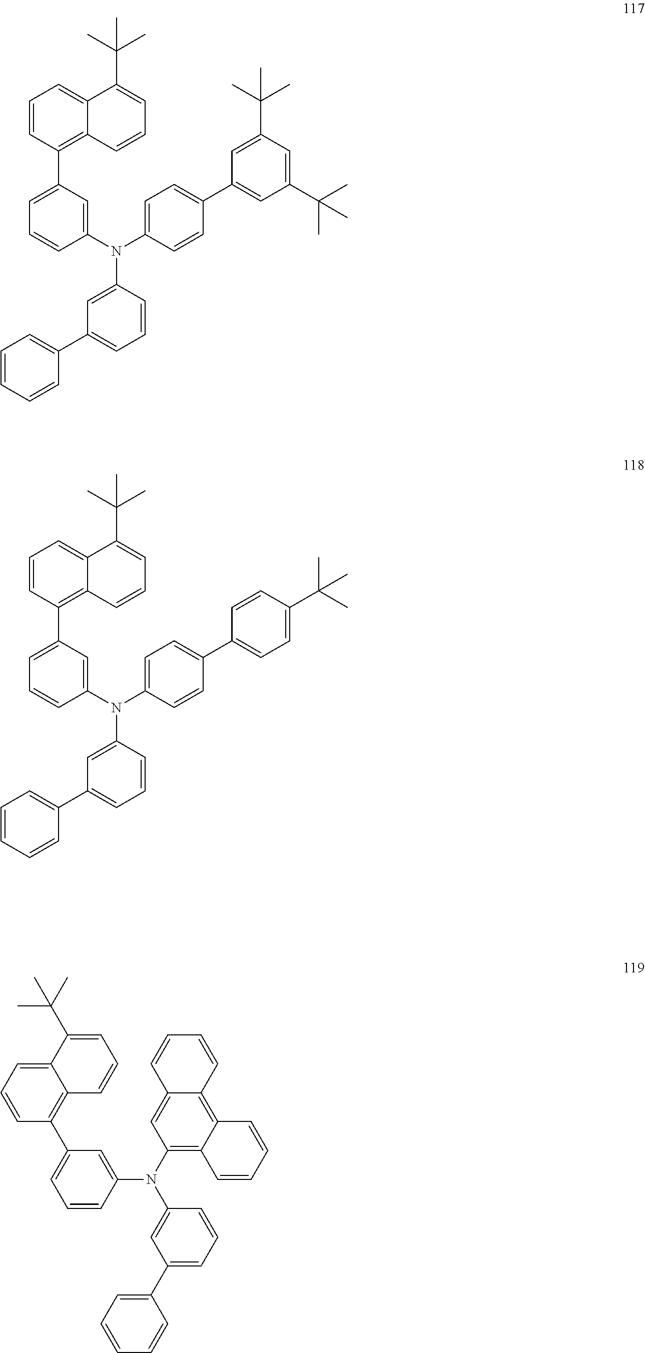 [SMILES: CC(C)(C)c1cc(-c2ccc(N(c3cccc(-c4ccccc4)c3)c3cccc(-c4cccc5c(C(C)(C)C)cccc45)c3)cc2)cc(C(C)(C)C)c1 ; CC(C)(C)c1ccc(-c2ccc(N(c3cccc(-c4ccccc4)c3)c3cccc(-c4cccc5c(C(C)(C)C)cccc45)c3)cc2)cc1 ; CC(C)(C)c1cccc2c(-c3cccc(N(c4cccc(-c5ccccc5)c4)c4cc5ccccc5c5ccccc45)c3)cccc12]

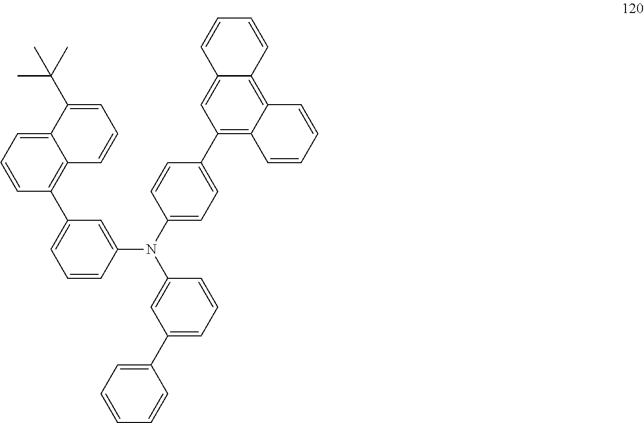 [SMILES: CC(C)(C)c1cccc2c(-c3cccc(N(c4ccc(-c5cc6ccccc6c6ccccc56)cc4)c4cccc(-c5ccccc5)c4)c3)cccc12]

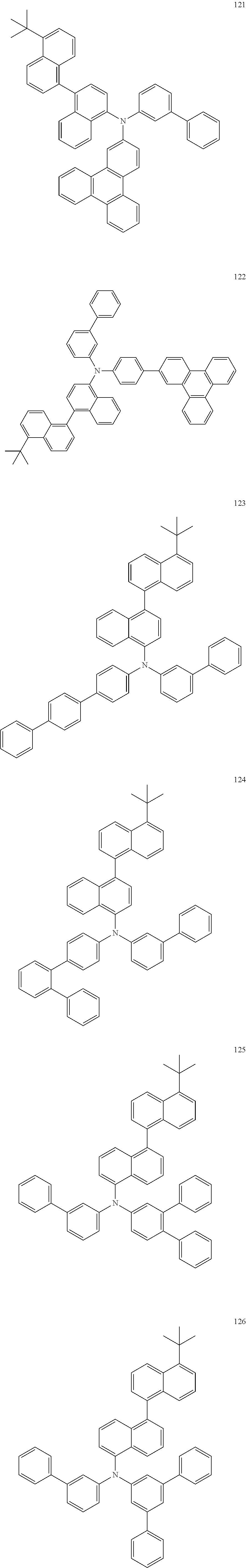

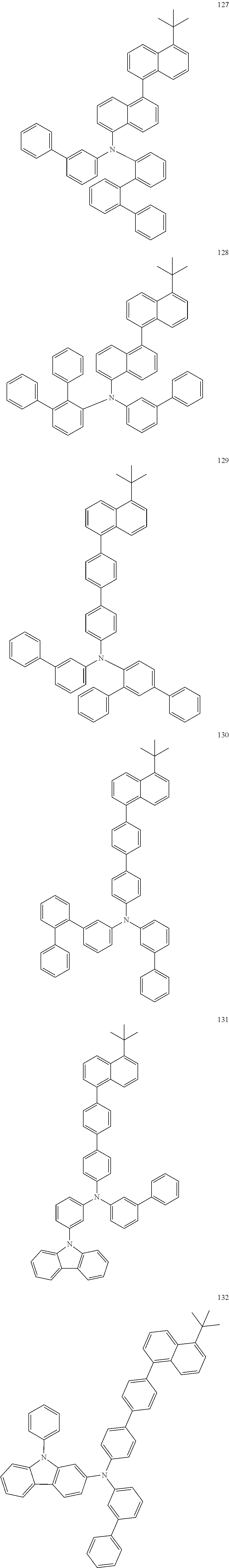

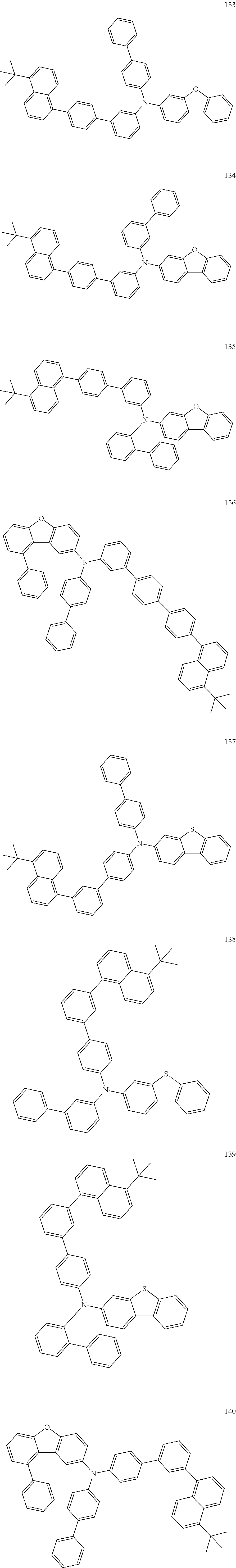

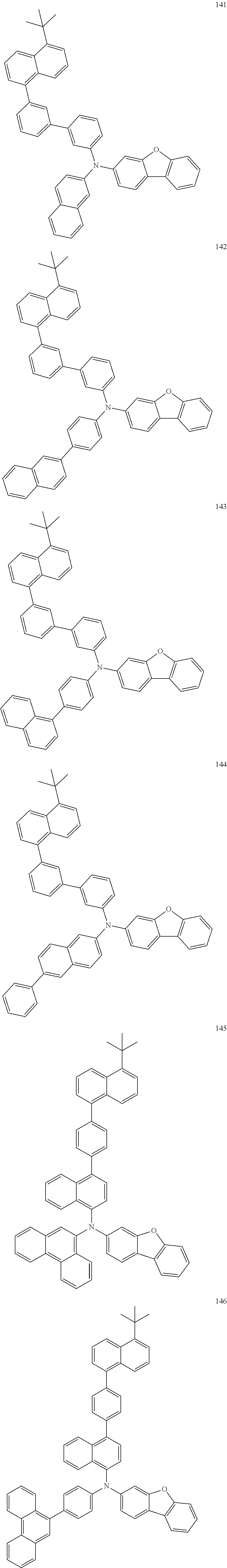

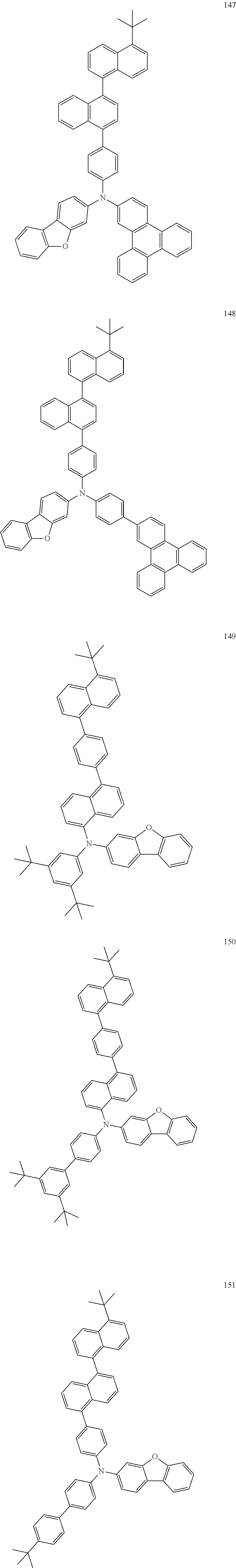

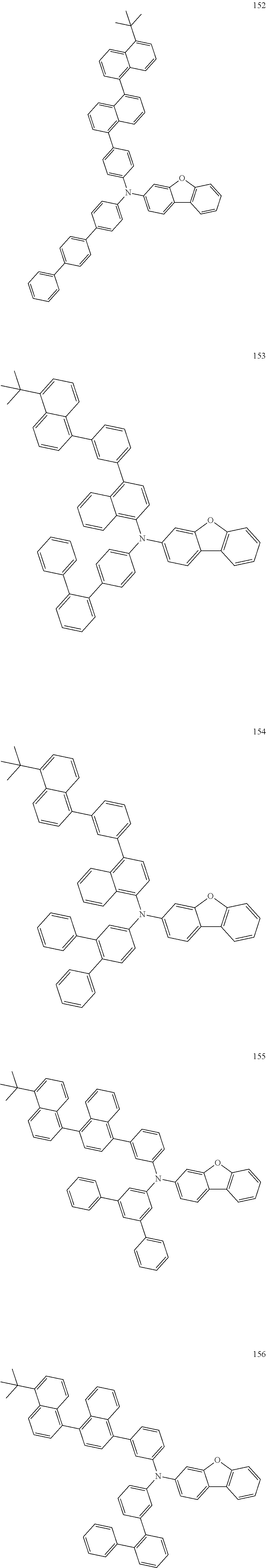

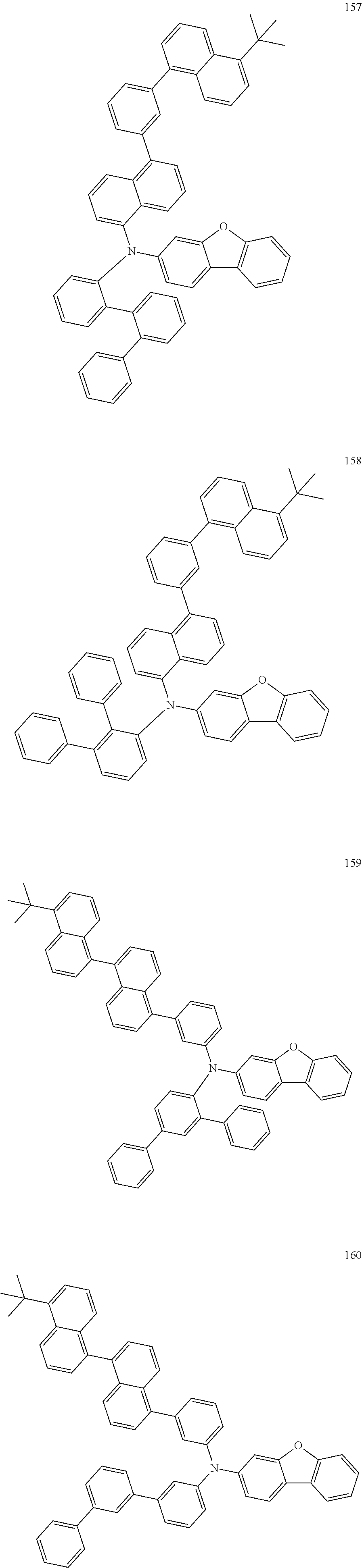 [SMILES: CC(C)(C)c1cccc2c(-c3cccc(-c4cccc5c(N(c6ccc7c(c6)oc6ccccc67)c6ccccc6-c6ccccc6-c6ccccc6)cccc45)c3)cccc12 ; CC(C)(C)c1cccc2c(-c3cccc(-c4cccc5c(N(c6ccc7c(c6)oc6ccccc67)c6cccc(-c7ccccc7)c6-c6ccccc6)cccc45)c3)cccc12 ; CC(C)(C)c1cccc2c(-c3cccc4c(-c5cccc(N(c6ccc7c(c6)oc6ccccc67)c6ccc(-c7ccccc7)cc6-c6ccccc6)c5)cccc34)cccc12 ; CC(C)(C)c1cccc2c(-c3cccc4c(-c5cccc(N(c6cccc(-c7cccc(-c8ccccc8)c7)c6)c6ccc7c(c6)oc6ccccc67)c5)cccc34)cccc12]

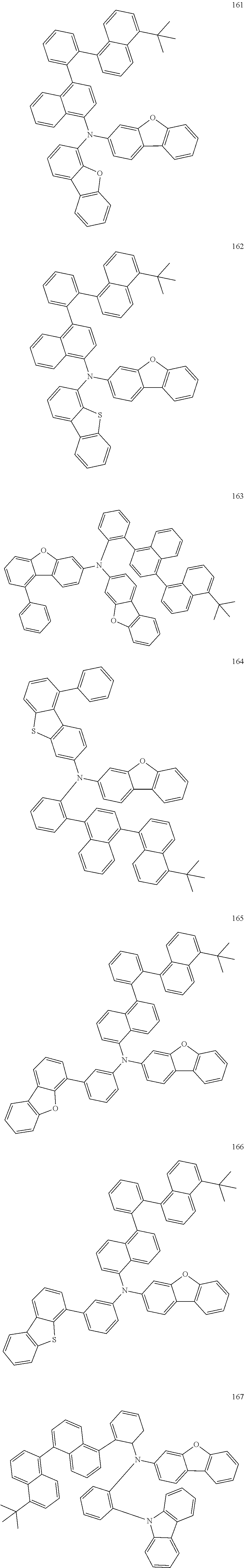

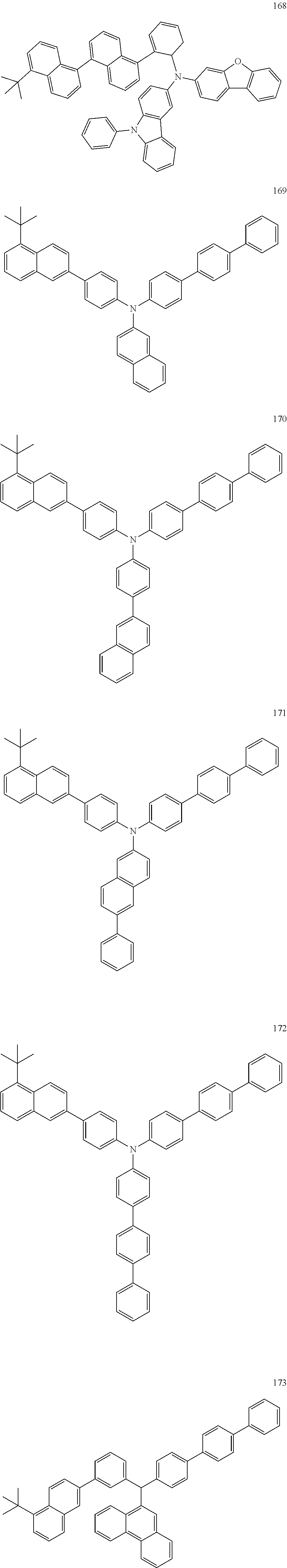

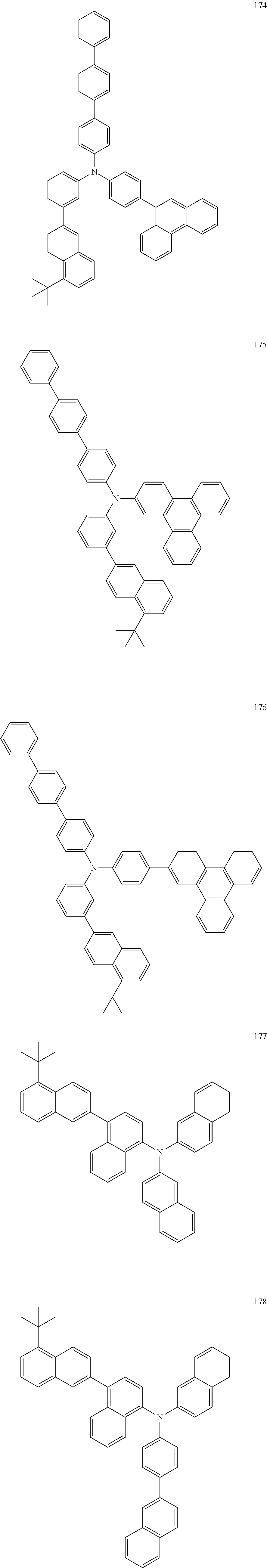 [SMILES: CC(C)(C)c1cccc2cc(-c3cccc(N(c4ccc(-c5ccc(-c6ccccc6)cc5)cc4)c4ccc(-c5cc6ccccc6c6ccccc56)cc4)c3)ccc12 ; CC(C)(C)c1cccc2cc(-c3cccc(N(c4ccc(-c5ccc(-c6ccccc6)cc5)cc4)c4ccc5c6ccccc6c6ccccc6c5c4)c3)ccc12 ; CC(C)(C)c1cccc2cc(-c3cccc(N(c4ccc(-c5ccc(-c6ccccc6)cc5)cc4)c4ccc(-c5ccc6c7ccccc7c7ccccc7c6c5)cc4)c3)ccc12 ; CC(C)(C)c1cccc2cc(-c3ccc(N(c4ccc5ccccc5c4)c4ccc5ccccc5c4)c4ccccc34)ccc12 ; CC(C)(C)c1cccc2cc(-c3ccc(N(c4ccc(-c5ccc6ccccc6c5)cc4)c4ccc5ccccc5c4)c4ccccc34)ccc12]

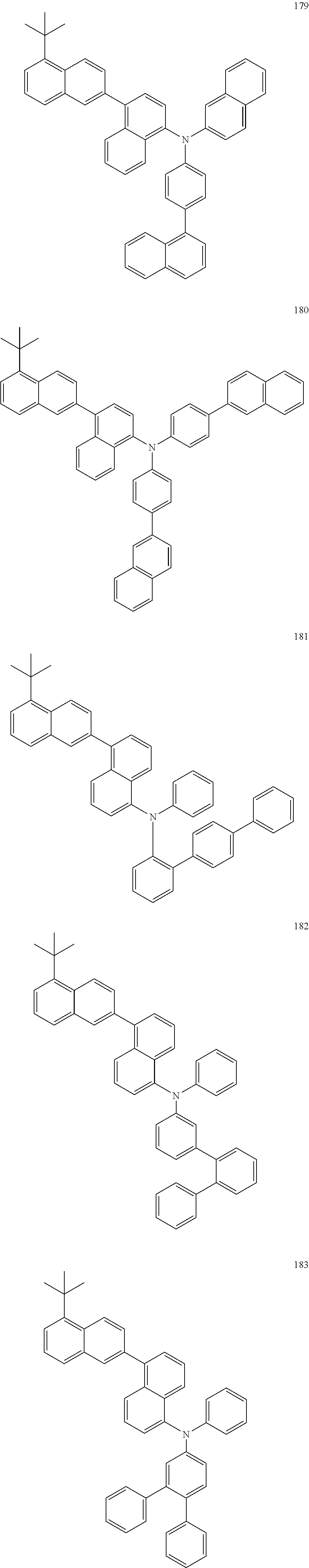 [SMILES: CC(C)(C)c1cccc2cc(-c3ccc(N(c4ccc(-c5cccc6ccccc56)cc4)c4ccc5ccccc5c4)c4ccccc34)ccc12 ; CC(C)(C)c1cccc2cc(-c3ccc(N(c4ccc(-c5ccc6ccccc6c5)cc4)c4ccc(-c5ccc6ccccc6c5)cc4)c4ccccc34)ccc12 ; CC(C)(C)c1cccc2cc(-c3cccc4c(N(c5ccccc5)c5ccccc5-c5ccc(-c6ccccc6)cc5)cccc34)ccc12 ; CC(C)(C)c1cccc2cc(-c3cccc4c(N(c5ccccc5)c5cccc(-c6ccccc6-c6ccccc6)c5)cccc34)ccc12 ; CC(C)(C)c1cccc2cc(-c3cccc4c(N(c5ccccc5)c5ccc(-c6ccccc6)c(-c6ccccc6)c5)cccc34)ccc12]

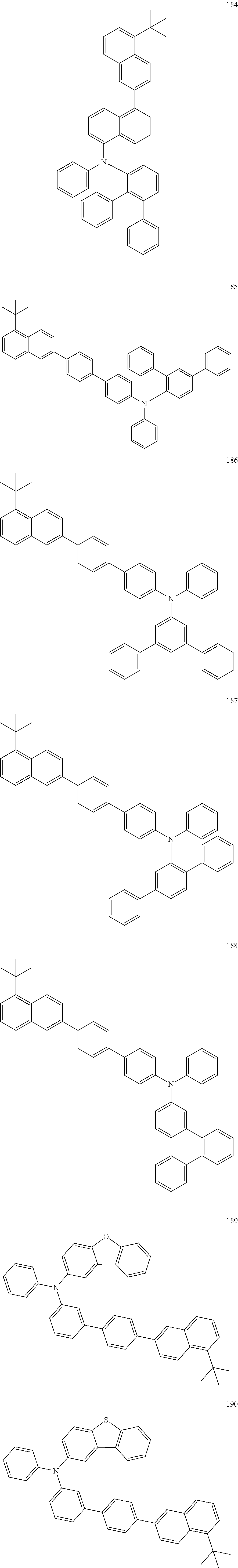

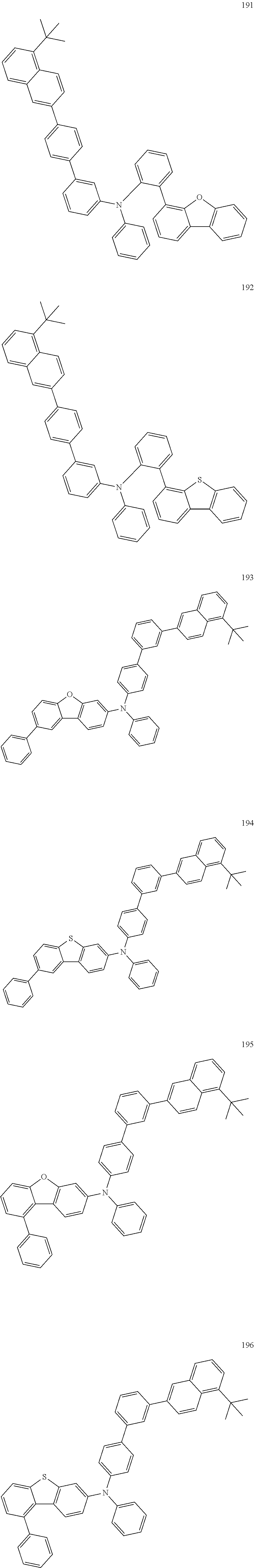

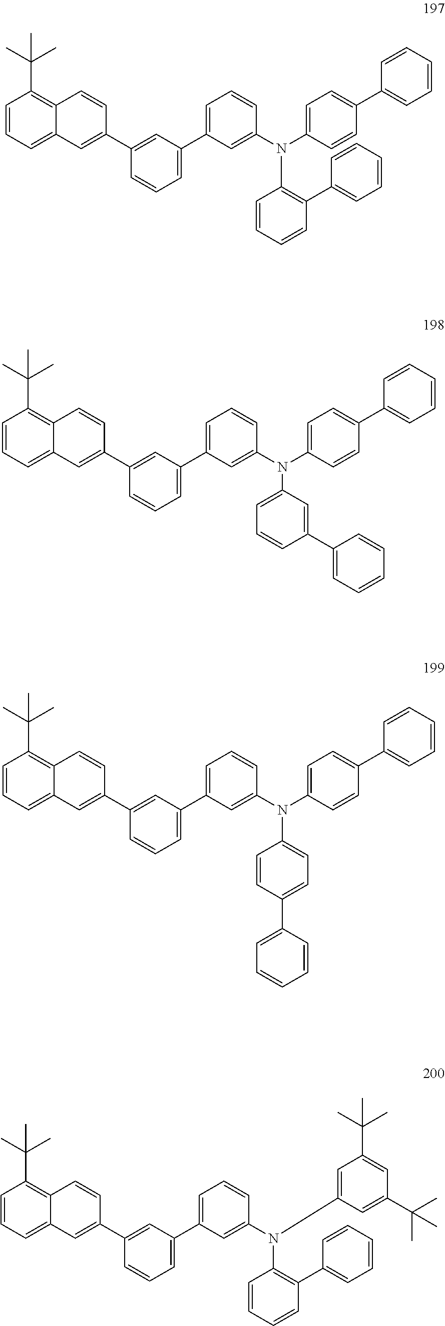 [SMILES: CC(C)(C)c1cccc2cc(-c3cccc(-c4cccc(N(c5ccc(-c6ccccc6)cc5)c5ccccc5-c5ccccc5)c4)c3)ccc12 ; CC(C)(C)c1cccc2cc(-c3cccc(-c4cccc(N(c5ccc(-c6ccccc6)cc5)c5cccc(-c6ccccc6)c5)c4)c3)ccc12 ; CC(C)(C)c1cccc2cc(-c3cccc(-c4cccc(N(c5ccc(-c6ccccc6)cc5)c5ccc(-c6ccccc6)cc5)c4)c3)ccc12 ; CC(C)(C)c1cc(N(c2cccc(-c3cccc(-c4ccc5c(C(C)(C)C)cccc5c4)c3)c2)c2ccccc2-c2ccccc2)cc(C(C)(C)C)c1]

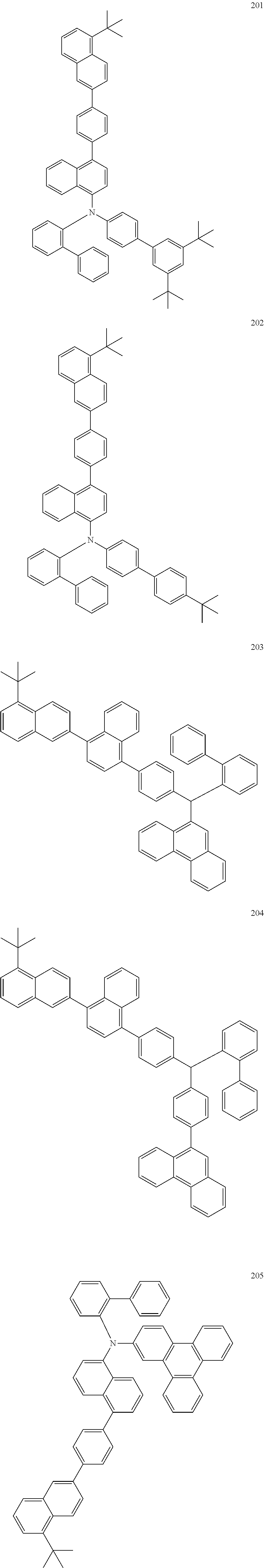

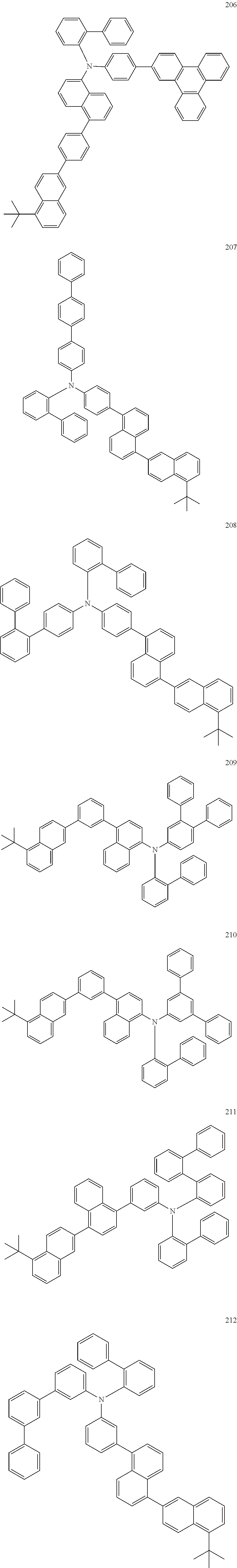

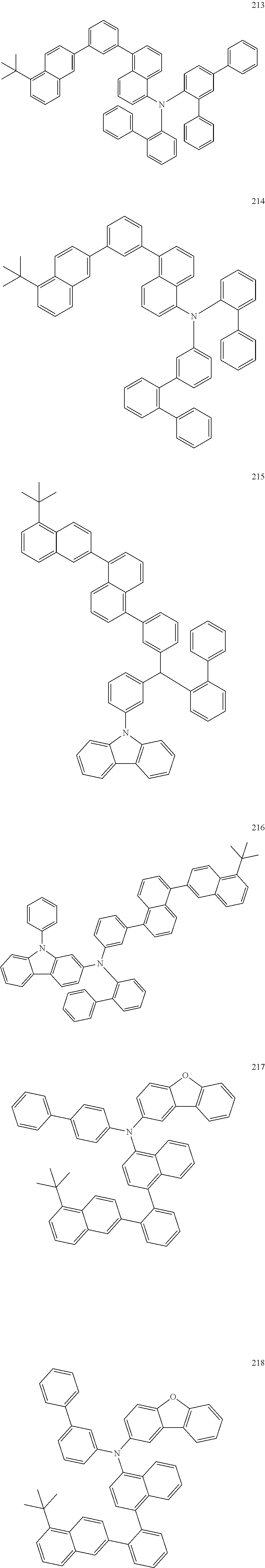

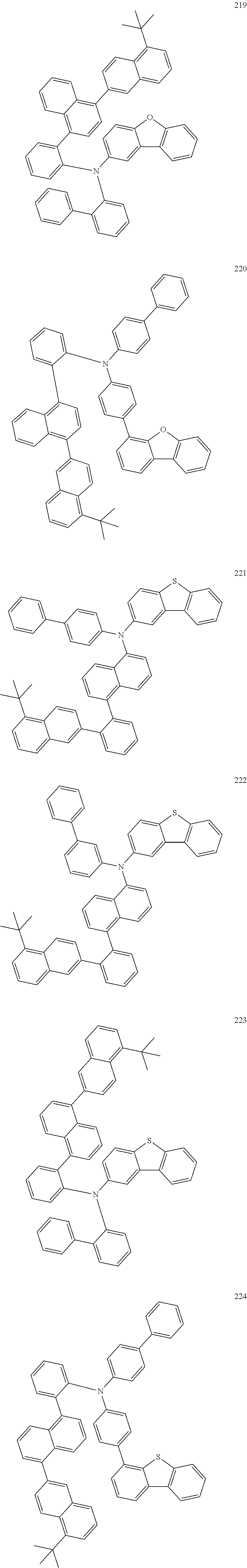

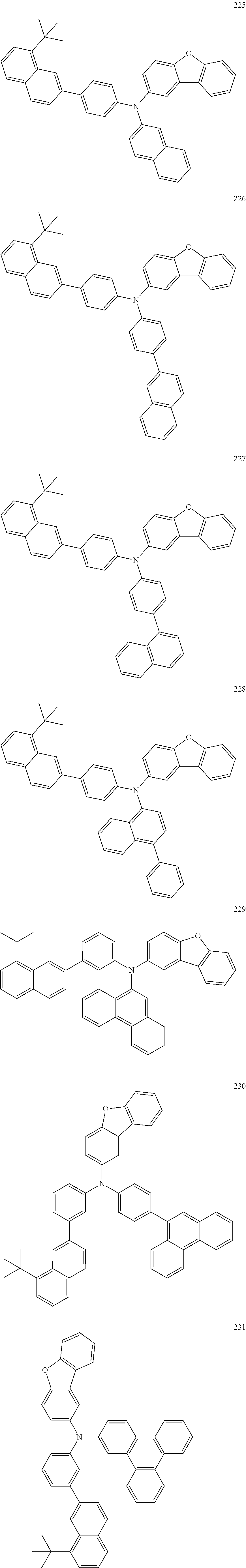

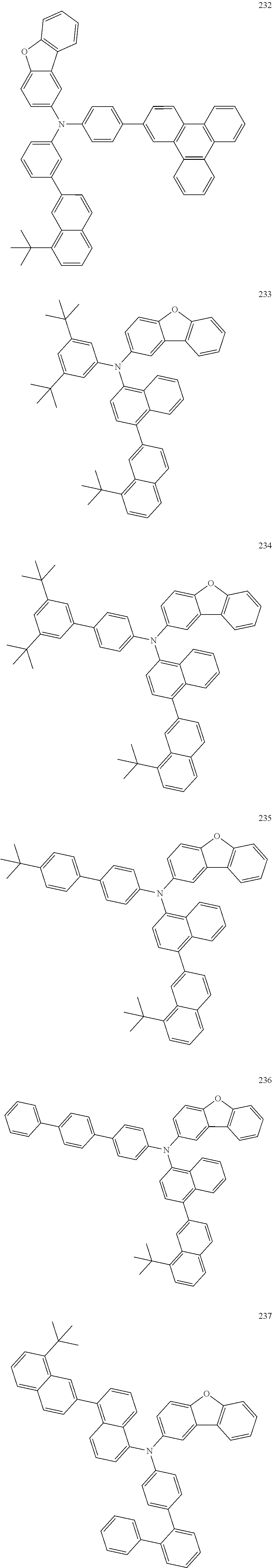

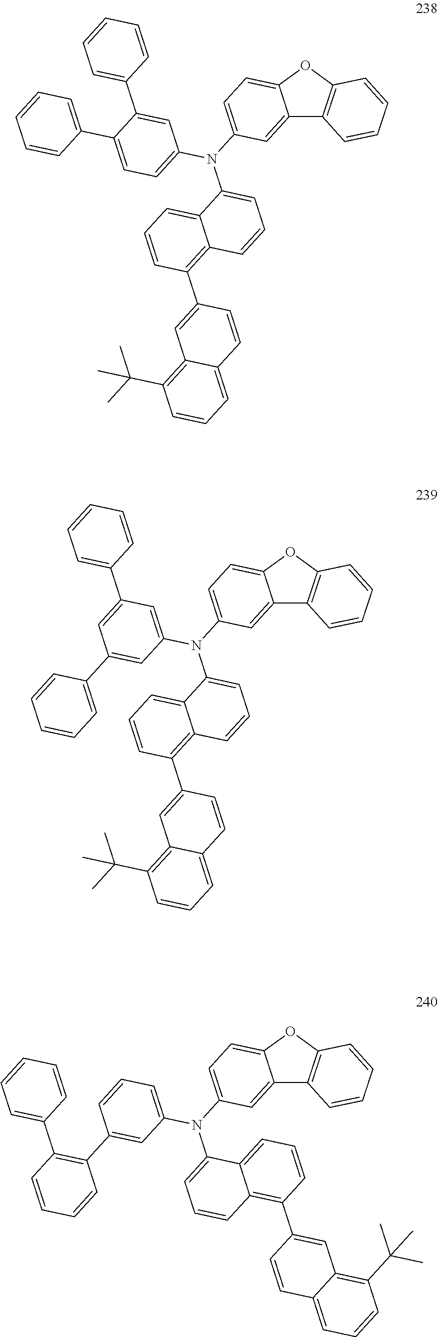 [SMILES: CC(C)(C)c1cccc2ccc(-c3cccc4c(N(c5ccc(-c6ccccc6)c(-c6ccccc6)c5)c5ccc6oc7ccccc7c6c5)cccc34)cc12 ; CC(C)(C)c1cccc2ccc(-c3cccc4c(N(c5cc(-c6ccccc6)cc(-c6ccccc6)c5)c5ccc6oc7ccccc7c6c5)cccc34)cc12 ; CC(C)(C)c1cccc2ccc(-c3cccc4c(N(c5cccc(-c6ccccc6-c6ccccc6)c5)c5ccc6oc7ccccc7c6c5)cccc34)cc12]

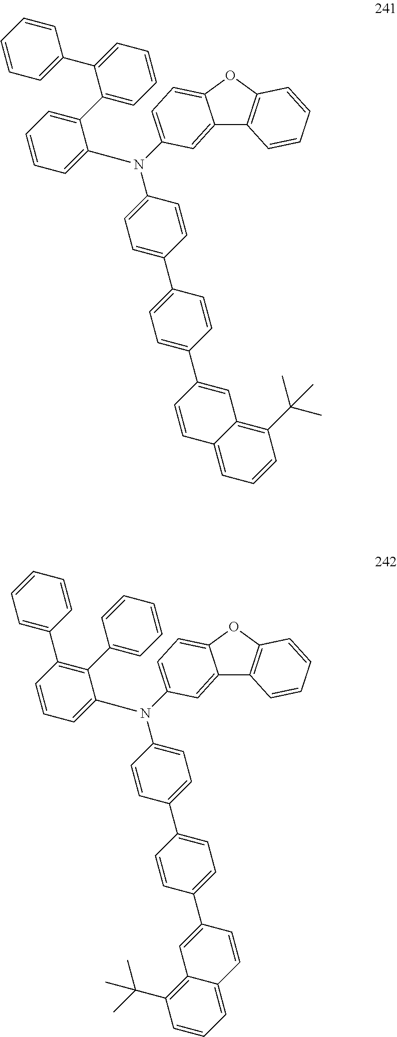 [SMILES: CC(C)(C)c1cccc2ccc(-c3ccc(-c4ccc(N(c5ccc6oc7ccccc7c6c5)c5ccccc5-c5ccccc5-c5ccccc5)cc4)cc3)cc12 ; CC(C)(C)c1cccc2ccc(-c3ccc(-c4ccc(N(c5ccc6oc7ccccc7c6c5)c5cccc(-c6ccccc6)c5-c5ccccc5)cc4)cc3)cc12]

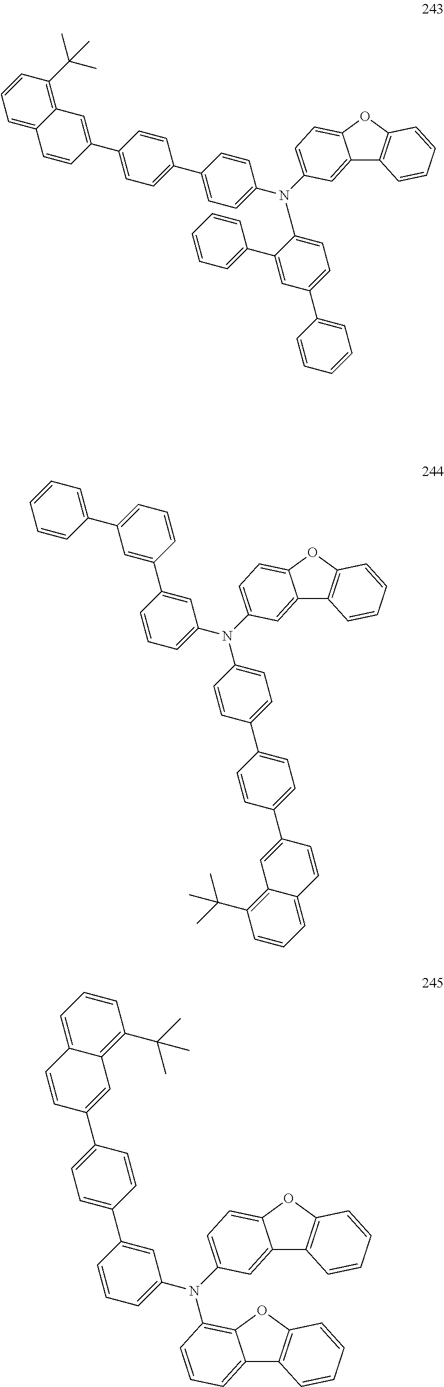 [SMILES: CC(C)(C)c1cccc2ccc(-c3ccc(-c4ccc(N(c5ccc6oc7ccccc7c6c5)c5ccc(-c6ccccc6)cc5-c5ccccc5)cc4)cc3)cc12 ; CC(C)(C)c1cccc2ccc(-c3ccc(-c4ccc(N(c5cccc(-c6cccc(-c7ccccc7)c6)c5)c5ccc6oc7ccccc7c6c5)cc4)cc3)cc12 ; CC(C)(C)c1cccc2ccc(-c3ccc(-c4cccc(N(c5ccc6oc7ccccc7c6c5)c5cccc6c5oc5ccccc56)c4)cc3)cc12]

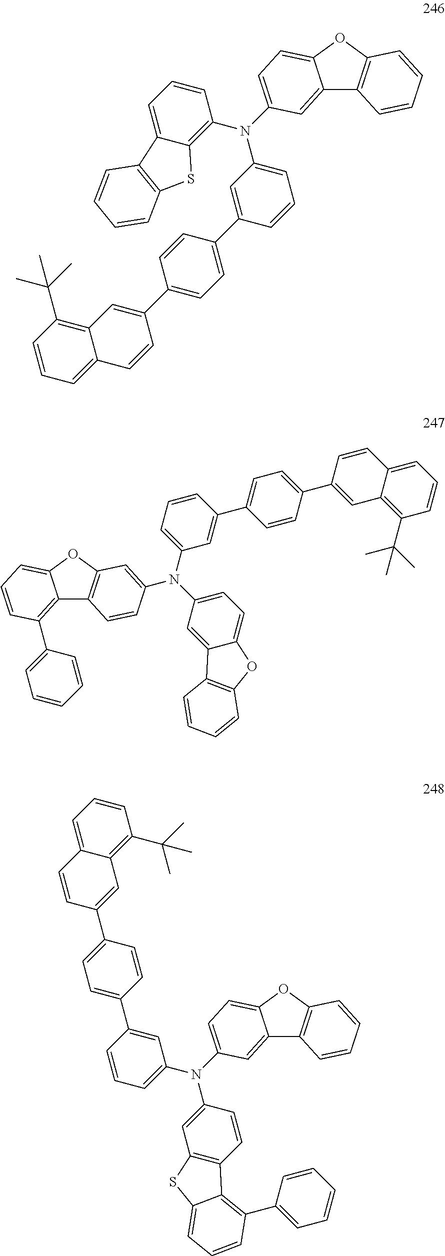 [SMILES: CC(C)(C)c1cccc2ccc(-c3ccc(-c4cccc(N(c5ccc6oc7ccccc7c6c5)c5cccc6c5sc5ccccc56)c4)cc3)cc12 ; CC(C)(C)c1cccc2ccc(-c3ccc(-c4cccc(N(c5ccc6c(c5)oc5cccc(-c7ccccc7)c56)c5ccc6oc7ccccc7c6c5)c4)cc3)cc12 ; CC(C)(C)c1cccc2ccc(-c3ccc(-c4cccc(N(c5ccc6c(c5)sc5cccc(-c7ccccc7)c56)c5ccc6oc7ccccc7c6c5)c4)cc3)cc12]

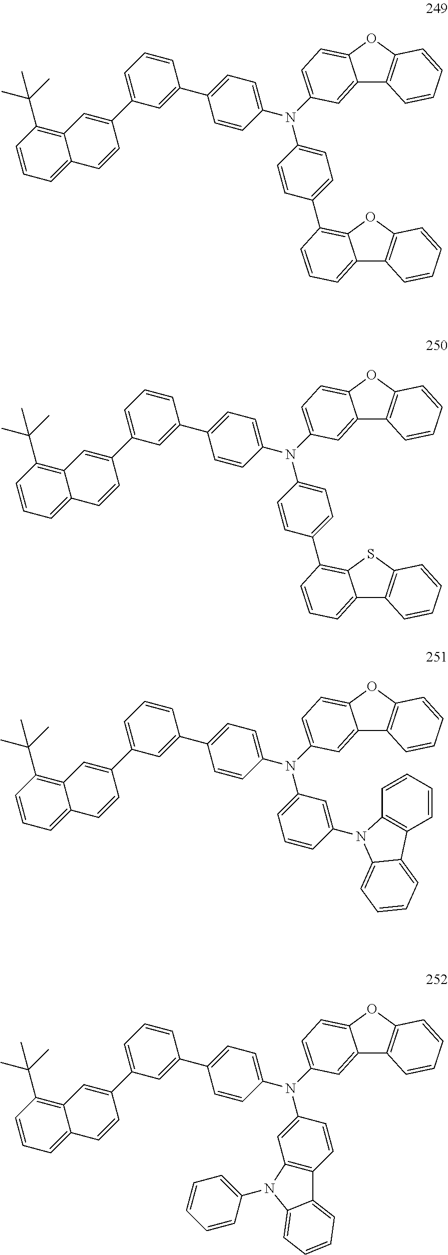 [SMILES: CC(C)(C)c1cccc2ccc(-c3cccc(-c4ccc(N(c5ccc(-c6cccc7c6oc6ccccc67)cc5)c5ccc6oc7ccccc7c6c5)cc4)c3)cc12 ; CC(C)(C)c1cccc2ccc(-c3cccc(-c4ccc(N(c5ccc(-c6cccc7c6sc6ccccc67)cc5)c5ccc6oc7ccccc7c6c5)cc4)c3)cc12 ; CC(C)(C)c1cccc2ccc(-c3cccc(-c4ccc(N(c5cccc(-n6c7ccccc7c7ccccc76)c5)c5ccc6oc7ccccc7c6c5)cc4)c3)cc12 ; CC(C)(C)c1cccc2ccc(-c3cccc(-c4ccc(N(c5ccc6oc7ccccc7c6c5)c5ccc6c7ccccc7n(-c7ccccc7)c6c5)cc4)c3)cc12]

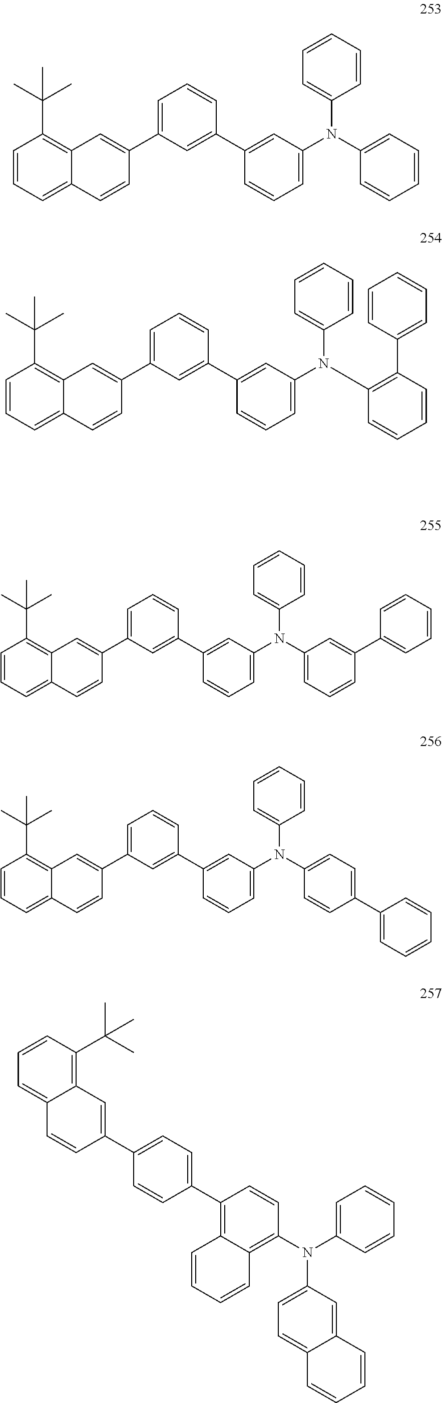 [SMILES: CC(C)(C)c1cccc2ccc(-c3cccc(-c4cccc(N(c5ccccc5)c5ccccc5)c4)c3)cc12 ; CC(C)(C)c1cccc2ccc(-c3cccc(-c4cccc(N(c5ccccc5)c5ccccc5-c5ccccc5)c4)c3)cc12 ; CC(C)(C)c1cccc2ccc(-c3cccc(-c4cccc(N(c5ccccc5)c5cccc(-c6ccccc6)c5)c4)c3)cc12 ; CC(C)(C)c1cccc2ccc(-c3cccc(-c4cccc(N(c5ccccc5)c5ccc(-c6ccccc6)cc5)c4)c3)cc12 ; CC(C)(C)c1cccc2ccc(-c3ccc(-c4ccc(N(c5ccccc5)c5ccc6ccccc6c5)c5ccccc45)cc3)cc12]

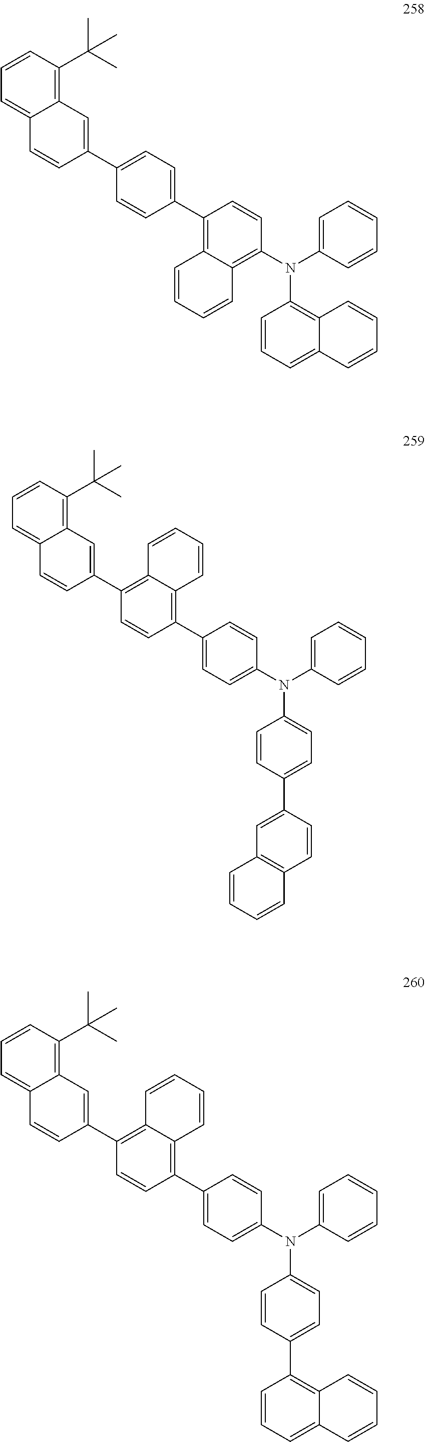 [SMILES: CC(C)(C)c1cccc2ccc(-c3ccc(-c4ccc(N(c5ccccc5)c5cccc6ccccc56)c5ccccc45)cc3)cc12 ; CC(C)(C)c1cccc2ccc(-c3ccc(-c4ccc(N(c5ccccc5)c5ccc(-c6ccc7ccccc7c6)cc5)cc4)c4ccccc34)cc12 ; CC(C)(C)c1cccc2ccc(-c3ccc(-c4ccc(N(c5ccccc5)c5ccc(-c6cccc7ccccc67)cc5)cc4)c4ccccc34)cc12]

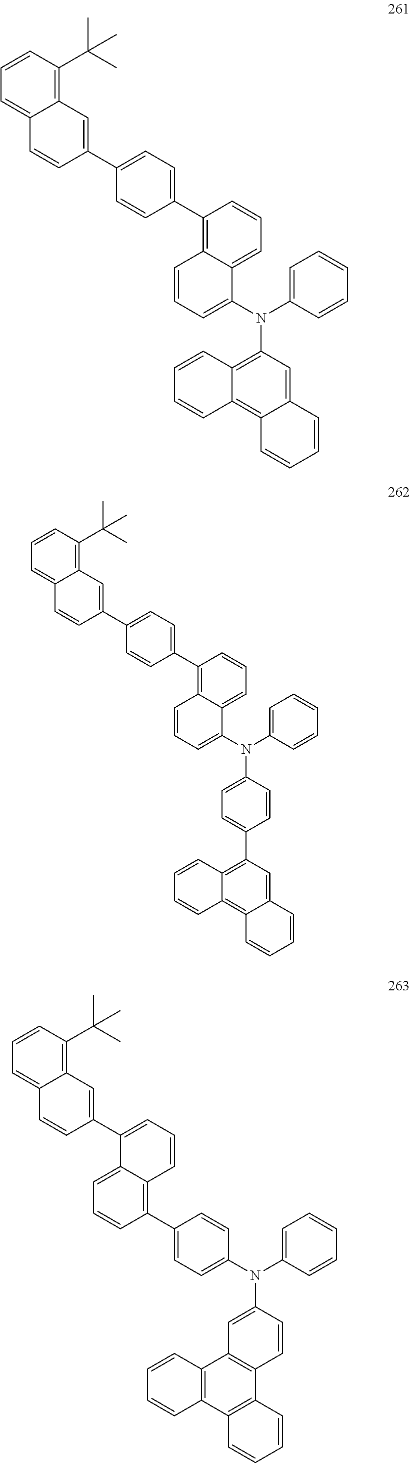 [SMILES: CC(C)(C)c1cccc2ccc(-c3ccc(-c4cccc5c(N(c6ccccc6)c6cc7ccccc7c7ccccc67)cccc45)cc3)cc12 ; CC(C)(C)c1cccc2ccc(-c3ccc(-c4cccc5c(N(c6ccccc6)c6ccc(-c7cc8ccccc8c8ccccc78)cc6)cccc45)cc3)cc12 ; CC(C)(C)c1cccc2ccc(-c3cccc4c(-c5ccc(N(c6ccccc6)c6ccc7c8ccccc8c8ccccc8c7c6)cc5)cccc34)cc12]

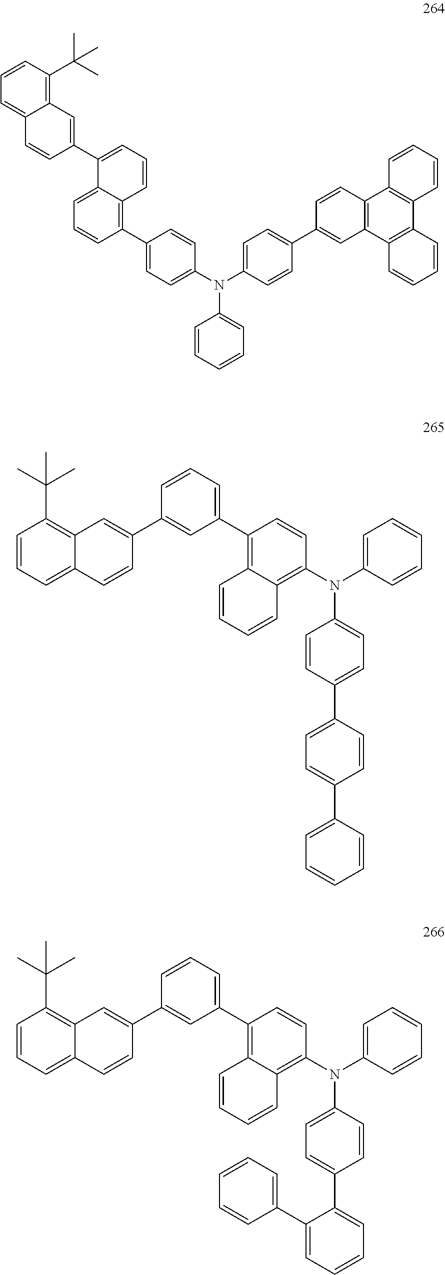 [SMILES: CC(C)(C)c1cccc2ccc(-c3cccc4c(-c5ccc(N(c6ccccc6)c6ccc(-c7ccc8c9ccccc9c9ccccc9c8c7)cc6)cc5)cccc34)cc12 ; CC(C)(C)c1cccc2ccc(-c3cccc(-c4ccc(N(c5ccccc5)c5ccc(-c6ccc(-c7ccccc7)cc6)cc5)c5ccccc45)c3)cc12 ; CC(C)(C)c1cccc2ccc(-c3cccc(-c4ccc(N(c5ccccc5)c5ccc(-c6ccccc6-c6ccccc6)cc5)c5ccccc45)c3)cc12]

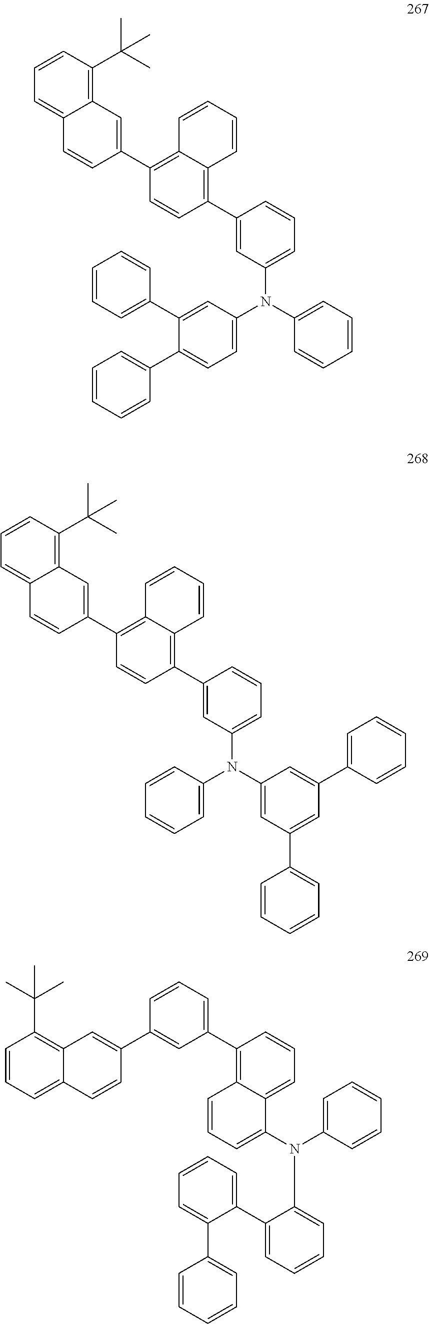 [SMILES: CC(C)(C)c1cccc2ccc(-c3ccc(-c4cccc(N(c5ccccc5)c5ccc(-c6ccccc6)c(-c6ccccc6)c5)c4)c4ccccc34)cc12 ; CC(C)(C)c1cccc2ccc(-c3ccc(-c4cccc(N(c5ccccc5)c5cc(-c6ccccc6)cc(-c6ccccc6)c5)c4)c4ccccc34)cc12 ; CC(C)(C)c1cccc2ccc(-c3cccc(-c4cccc5c(N(c6ccccc6)c6ccccc6-c6ccccc6-c6ccccc6)cccc45)c3)cc12]

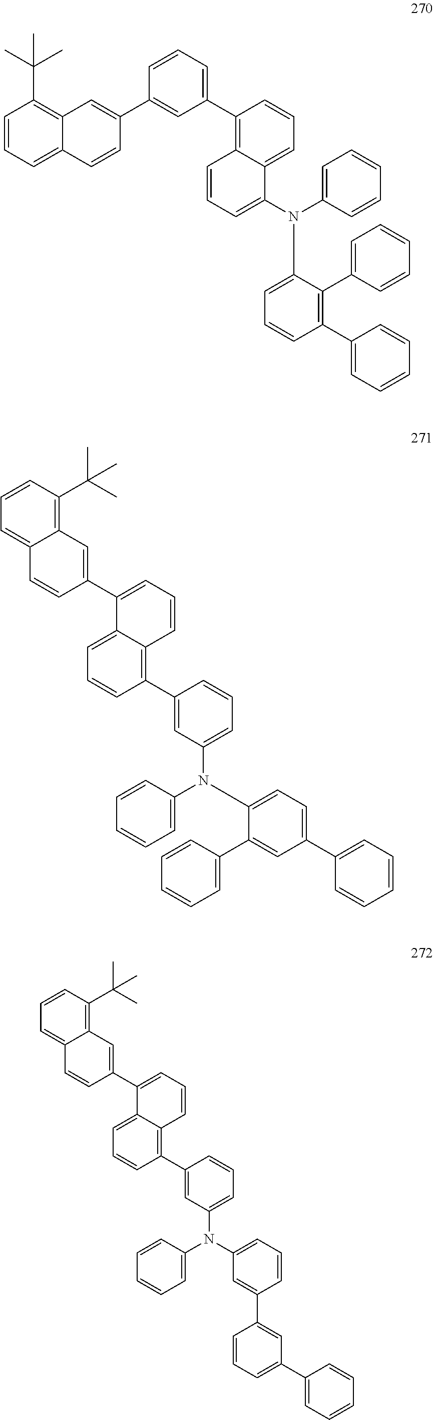 [SMILES: CC(C)(C)c1cccc2ccc(-c3cccc(-c4cccc5c(N(c6ccccc6)c6cccc(-c7ccccc7)c6-c6ccccc6)cccc45)c3)cc12 ; CC(C)(C)c1cccc2ccc(-c3cccc4c(-c5cccc(N(c6ccccc6)c6ccc(-c7ccccc7)cc6-c6ccccc6)c5)cccc34)cc12 ; CC(C)(C)c1cccc2ccc(-c3cccc4c(-c5cccc(N(c6ccccc6)c6cccc(-c7cccc(-c8ccccc8)c7)c6)c5)cccc34)cc12]

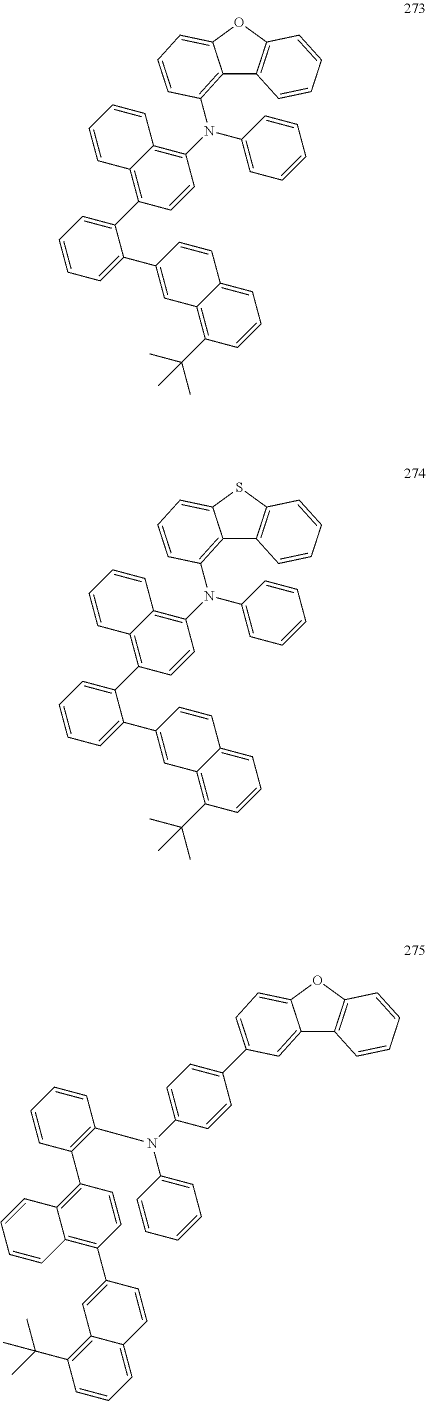 [SMILES: CC(C)(C)c1cccc2ccc(-c3ccccc3-c3ccc(N(c4ccccc4)c4cccc5oc6ccccc6c45)c4ccccc34)cc12 ; CC(C)(C)c1cccc2ccc(-c3ccccc3-c3ccc(N(c4ccccc4)c4cccc5sc6ccccc6c45)c4ccccc34)cc12 ; CC(C)(C)c1cccc2ccc(-c3ccc(-c4ccccc4N(c4ccccc4)c4ccc(-c5ccc6oc7ccccc7c6c5)cc4)c4ccccc34)cc12]

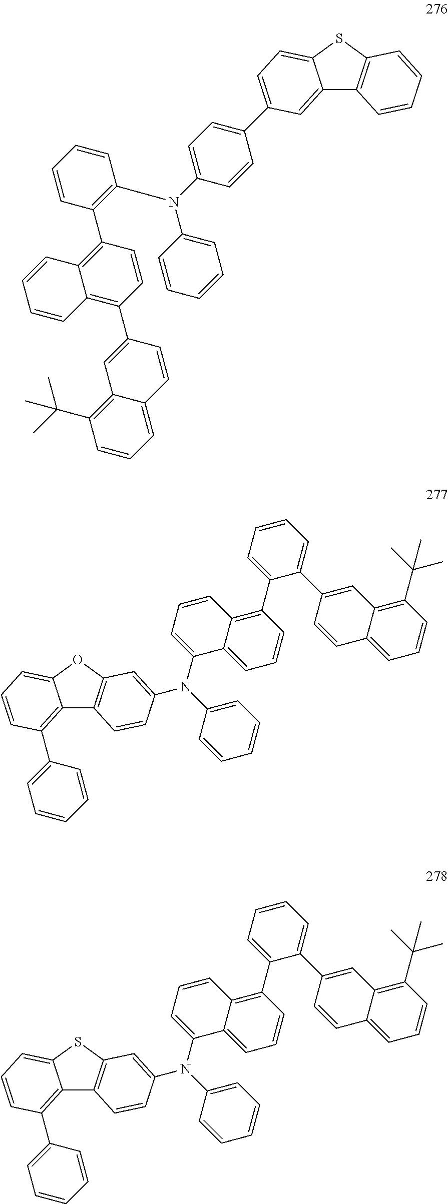 [SMILES: CC(C)(C)c1cccc2ccc(-c3ccc(-c4ccccc4N(c4ccccc4)c4ccc(-c5ccc6sc7ccccc7c6c5)cc4)c4ccccc34)cc12 ; CC(C)(C)c1cccc2ccc(-c3ccccc3-c3cccc4c(N(c5ccccc5)c5ccc6c(c5)oc5cccc(-c7ccccc7)c56)cccc34)cc12 ; CC(C)(C)c1cccc2ccc(-c3ccccc3-c3cccc4c(N(c5ccccc5)c5ccc6c(c5)sc5cccc(-c7ccccc7)c56)cccc34)cc12]

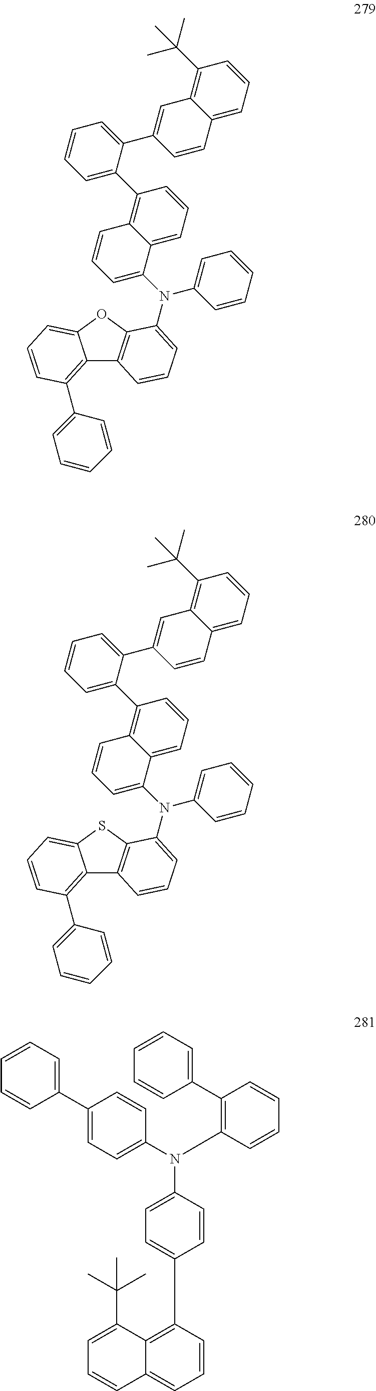 [SMILES: CC(C)(C)c1cccc2ccc(-c3ccccc3-c3cccc4c(N(c5ccccc5)c5cccc6c5oc5cccc(-c7ccccc7)c56)cccc34)cc12 ; CC(C)(C)c1cccc2ccc(-c3ccccc3-c3cccc4c(N(c5ccccc5)c5cccc6c5sc5cccc(-c7ccccc7)c56)cccc34)cc12 ; CC(C)(C)c1cccc2cccc(-c3ccc(N(c4ccc(-c5ccccc5)cc4)c4ccccc4-c4ccccc4)cc3)c12]

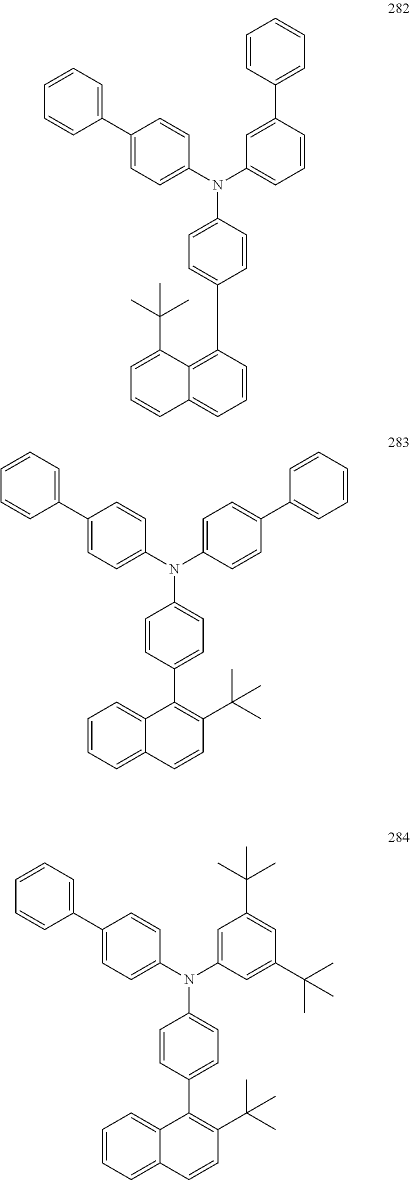 [SMILES: CC(C)(C)c1cccc2cccc(-c3ccc(N(c4ccc(-c5ccccc5)cc4)c4cccc(-c5ccccc5)c4)cc3)c12 ; CC(C)(C)c1ccc2ccccc2c1-c1ccc(N(c2ccc(-c3ccccc3)cc2)c2ccc(-c3ccccc3)cc2)cc1 ; CC(C)(C)c1cc(N(c2ccc(-c3ccccc3)cc2)c2ccc(-c3c(C(C)(C)C)ccc4ccccc34)cc2)cc(C(C)(C)C)c1]

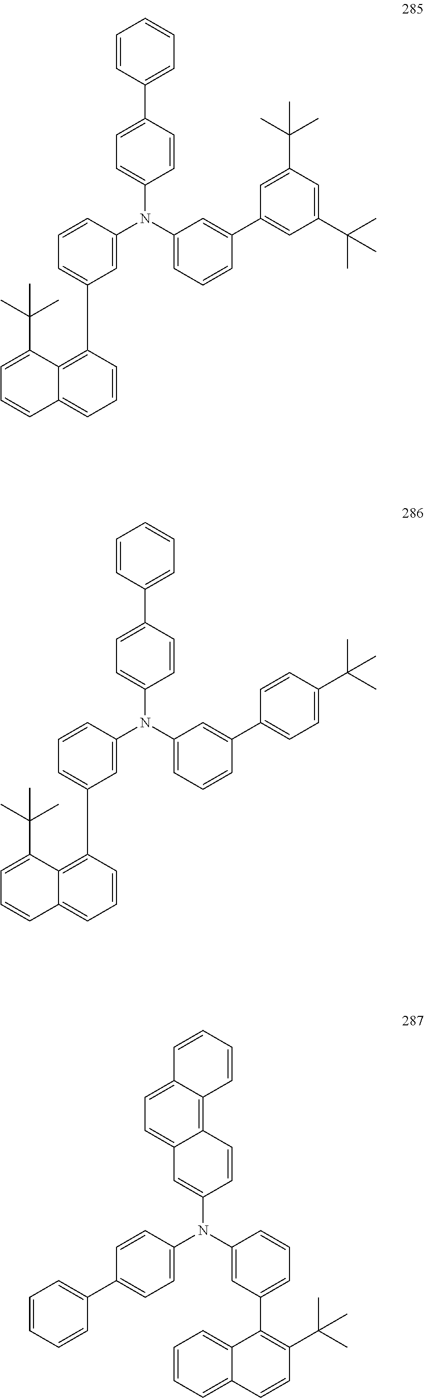 [SMILES: CC(C)(C)c1cc(-c2cccc(N(c3ccc(-c4ccccc4)cc3)c3cccc(-c4cccc5cccc(C(C)(C)C)c45)c3)c2)cc(C(C)(C)C)c1 ; CC(C)(C)c1ccc(-c2cccc(N(c3ccc(-c4ccccc4)cc3)c3cccc(-c4cccc5cccc(C(C)(C)C)c45)c3)c2)cc1 ; CC(C)(C)c1ccc2ccccc2c1-c1cccc(N(c2ccc(-c3ccccc3)cc2)c2ccc3c(ccc4ccccc43)c2)c1]

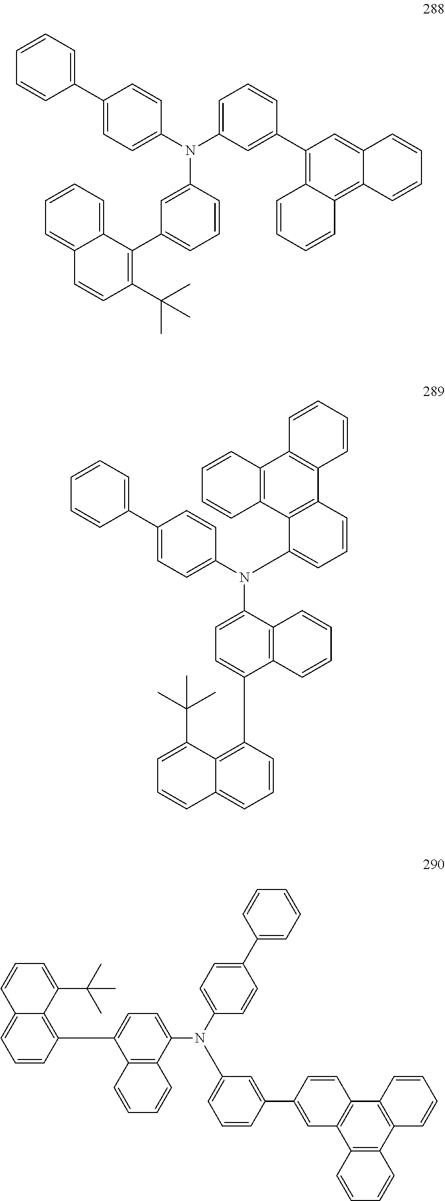 [SMILES: CC(C)(C)c1ccc2ccccc2c1-c1cccc(N(c2ccc(-c3ccccc3)cc2)c2cccc(-c3cc4ccccc4c4ccccc34)c2)c1 ; CC(C)(C)c1cccc2cccc(-c3ccc(N(c4ccc(-c5ccccc5)cc4)c4cccc5c6ccccc6c6ccccc6c45)c4ccccc34)c12 ; CC(C)(C)c1cccc2cccc(-c3ccc(N(c4ccc(-c5ccccc5)cc4)c4cccc(-c5ccc6c7ccccc7c7ccccc7c6c5)c4)c4ccccc34)c12]

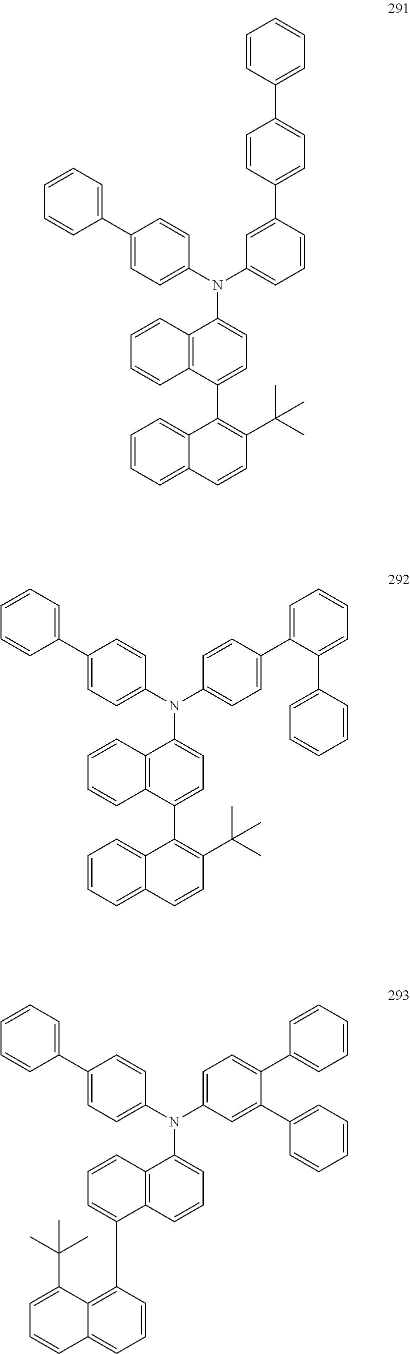 [SMILES: CC(C)(C)c1ccc2ccccc2c1-c1ccc(N(c2ccc(-c3ccccc3)cc2)c2cccc(-c3ccc(-c4ccccc4)cc3)c2)c2ccccc12 ; CC(C)(C)c1ccc2ccccc2c1-c1ccc(N(c2ccc(-c3ccccc3)cc2)c2ccc(-c3ccccc3-c3ccccc3)cc2)c2ccccc12 ; CC(C)(C)c1cccc2cccc(-c3cccc4c(N(c5ccc(-c6ccccc6)cc5)c5ccc(-c6ccccc6)c(-c6ccccc6)c5)cccc34)c12]

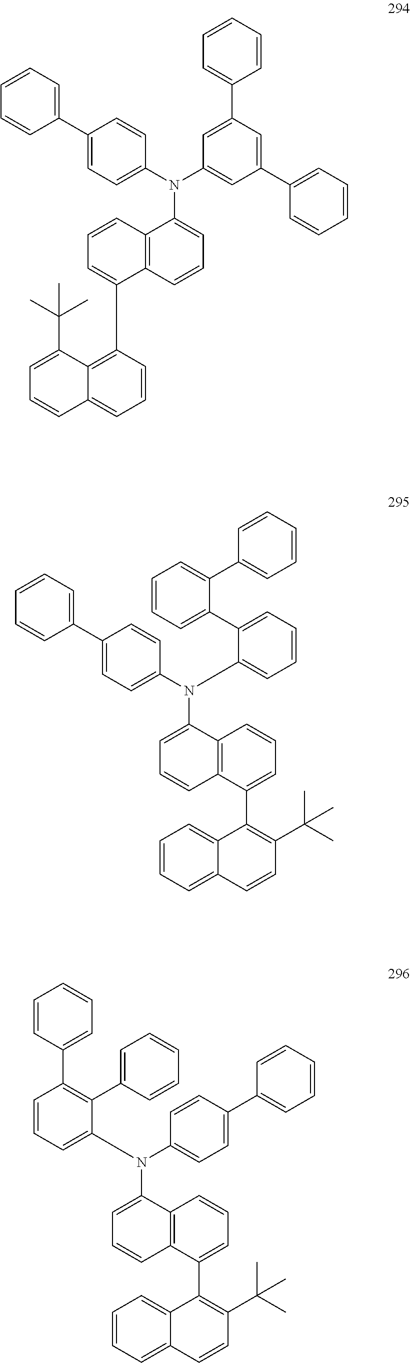 [SMILES: CC(C)(C)c1cccc2cccc(-c3cccc4c(N(c5ccc(-c6ccccc6)cc5)c5cc(-c6ccccc6)cc(-c6ccccc6)c5)cccc34)c12 ; CC(C)(C)c1ccc2ccccc2c1-c1cccc2c(N(c3ccc(-c4ccccc4)cc3)c3ccccc3-c3ccccc3-c3ccccc3)cccc12 ; CC(C)(C)c1ccc2ccccc2c1-c1cccc2c(N(c3ccc(-c4ccccc4)cc3)c3cccc(-c4ccccc4)c3-c3ccccc3)cccc12]

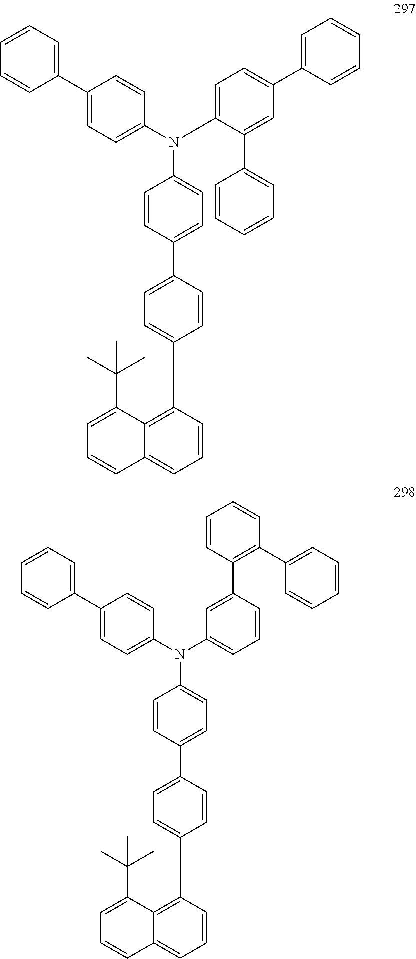 [SMILES: CC(C)(C)c1cccc2cccc(-c3ccc(-c4ccc(N(c5ccc(-c6ccccc6)cc5)c5ccc(-c6ccccc6)cc5-c5ccccc5)cc4)cc3)c12 ; CC(C)(C)c1cccc2cccc(-c3ccc(-c4ccc(N(c5ccc(-c6ccccc6)cc5)c5cccc(-c6ccccc6-c6ccccc6)c5)cc4)cc3)c12]

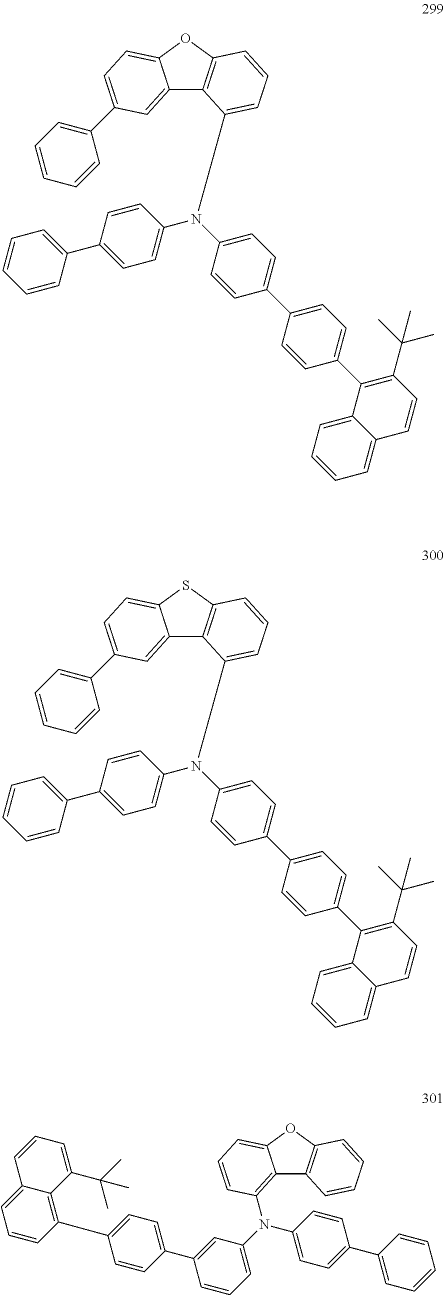 [SMILES: CC(C)(C)c1ccc2ccccc2c1-c1ccc(-c2ccc(N(c3ccc(-c4ccccc4)cc3)c3cccc4oc5ccc(-c6ccccc6)cc5c34)cc2)cc1 ; CC(C)(C)c1ccc2ccccc2c1-c1ccc(-c2ccc(N(c3ccc(-c4ccccc4)cc3)c3cccc4sc5ccc(-c6ccccc6)cc5c34)cc2)cc1 ; CC(C)(C)c1cccc2cccc(-c3ccc(-c4cccc(N(c5ccc(-c6ccccc6)cc5)c5cccc6oc7ccccc7c56)c4)cc3)c12]

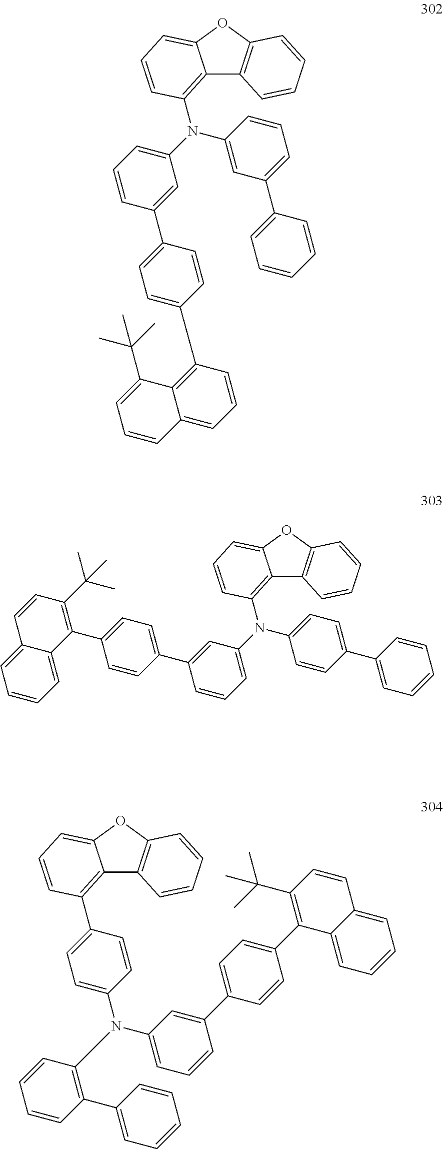 [SMILES: CC(C)(C)c1cccc2cccc(-c3ccc(-c4cccc(N(c5cccc(-c6ccccc6)c5)c5cccc6oc7ccccc7c56)c4)cc3)c12 ; CC(C)(C)c1ccc2ccccc2c1-c1ccc(-c2cccc(N(c3ccc(-c4ccccc4)cc3)c3cccc4oc5ccccc5c34)c2)cc1 ; CC(C)(C)c1ccc2ccccc2c1-c1ccc(-c2cccc(N(c3ccc(-c4cccc5oc6ccccc6c45)cc3)c3ccccc3-c3ccccc3)c2)cc1]

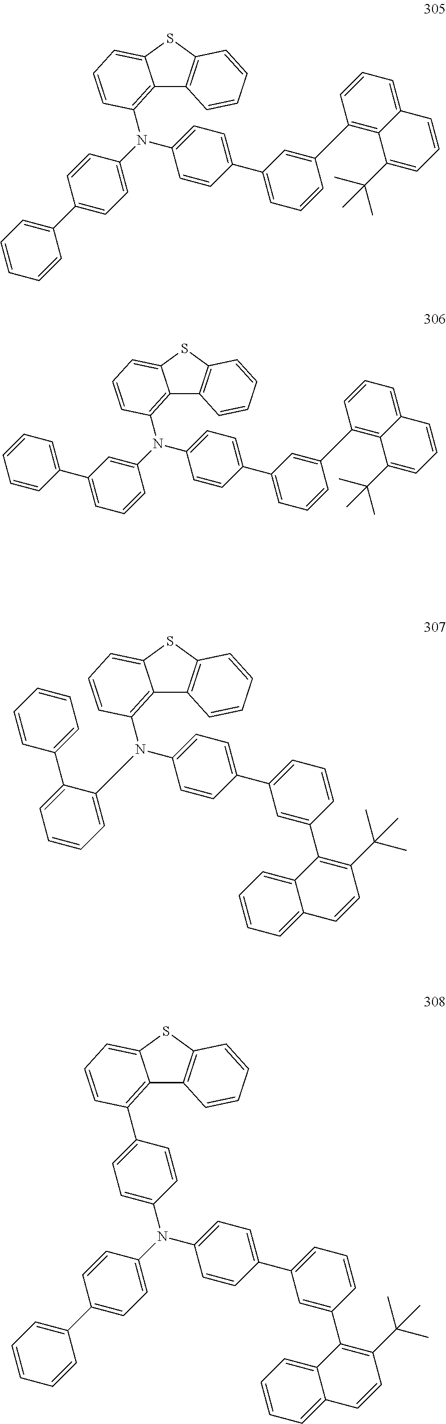 [SMILES: CC(C)(C)c1cccc2cccc(-c3cccc(-c4ccc(N(c5ccc(-c6ccccc6)cc5)c5cccc6sc7ccccc7c56)cc4)c3)c12 ; CC(C)(C)c1cccc2cccc(-c3cccc(-c4ccc(N(c5cccc(-c6ccccc6)c5)c5cccc6sc7ccccc7c56)cc4)c3)c12 ; CC(C)(C)c1ccc2ccccc2c1-c1cccc(-c2ccc(N(c3ccccc3-c3ccccc3)c3cccc4sc5ccccc5c34)cc2)c1 ; CC(C)(C)c1ccc2ccccc2c1-c1cccc(-c2ccc(N(c3ccc(-c4ccccc4)cc3)c3ccc(-c4cccc5sc6ccccc6c45)cc3)cc2)c1]

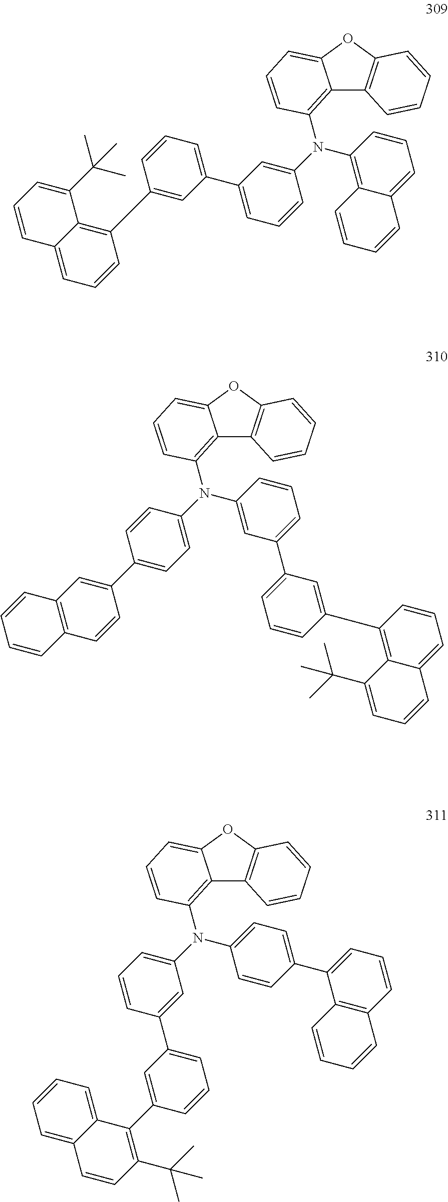 [SMILES: CC(C)(C)c1cccc2cccc(-c3cccc(-c4cccc(N(c5cccc6ccccc56)c5cccc6oc7ccccc7c56)c4)c3)c12 ; CC(C)(C)c1cccc2cccc(-c3cccc(-c4cccc(N(c5ccc(-c6ccc7ccccc7c6)cc5)c5cccc6oc7ccccc7c56)c4)c3)c12 ; CC(C)(C)c1ccc2ccccc2c1-c1cccc(-c2cccc(N(c3ccc(-c4cccc5ccccc45)cc3)c3cccc4oc5ccccc5c34)c2)c1]

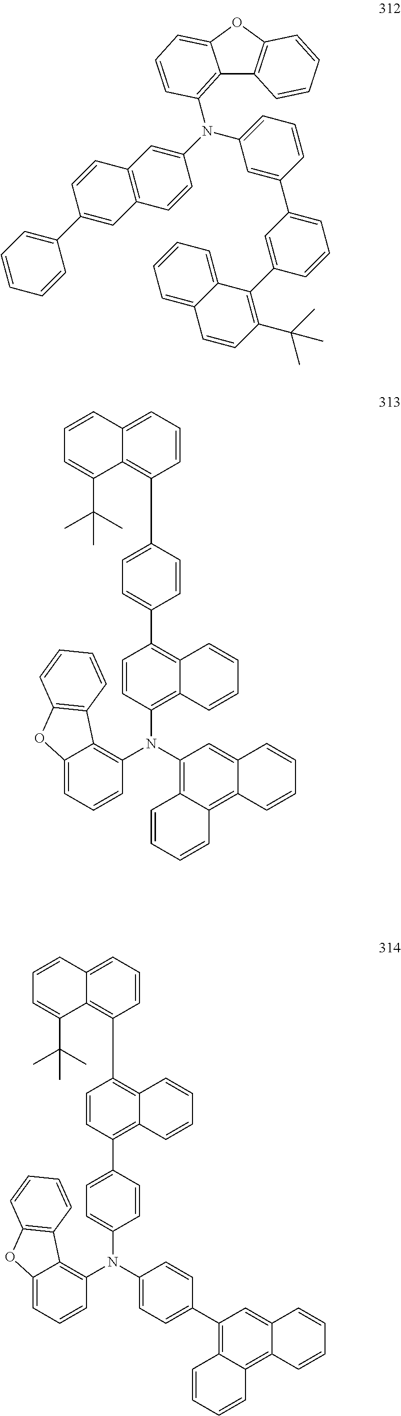 [SMILES: CC(C)(C)c1ccc2ccccc2c1-c1cccc(-c2cccc(N(c3ccc4cc(-c5ccccc5)ccc4c3)c3cccc4oc5ccccc5c34)c2)c1 ; CC(C)(C)c1cccc2cccc(-c3ccc(-c4ccc(N(c5cc6ccccc6c6ccccc56)c5cccc6oc7ccccc7c56)c5ccccc45)cc3)c12 ; CC(C)(C)c1cccc2cccc(-c3ccc(-c4ccc(N(c5ccc(-c6cc7ccccc7c7ccccc67)cc5)c5cccc6oc7ccccc7c56)cc4)c4ccccc34)c12]

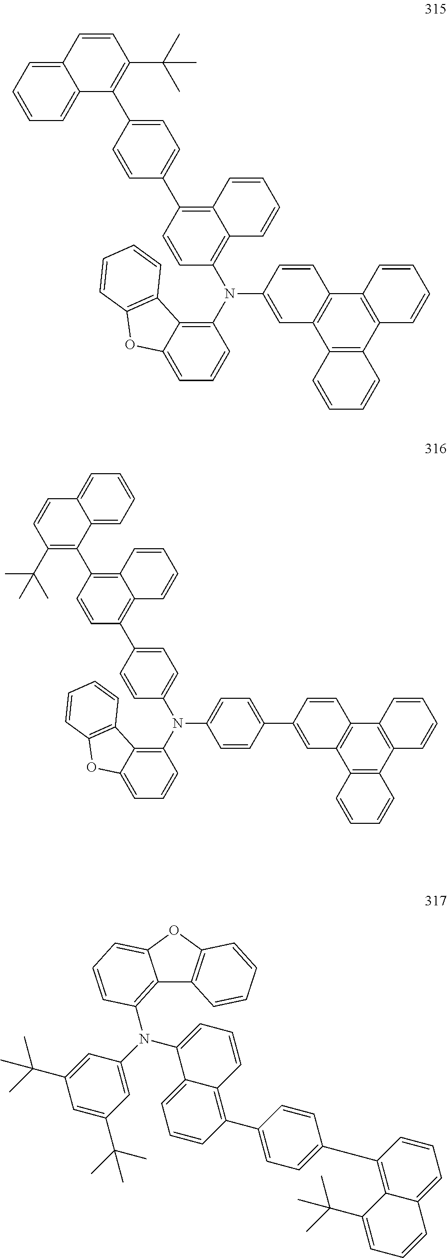 [SMILES: CC(C)(C)c1ccc2ccccc2c1-c1ccc(-c2ccc(N(c3ccc4c5ccccc5c5ccccc5c4c3)c3cccc4oc5ccccc5c34)c3ccccc23)cc1 ; CC(C)(C)c1ccc2ccccc2c1-c1ccc(-c2ccc(N(c3ccc(-c4ccc5c6ccccc6c6ccccc6c5c4)cc3)c3cccc4oc5ccccc5c34)cc2)c2ccccc12 ; CC(C)(C)c1cc(N(c2cccc3c(-c4ccc(-c5cccc6cccc(C(C)(C)C)c56)cc4)cccc23)c2cccc3oc4ccccc4c23)cc(C(C)(C)C)c1]

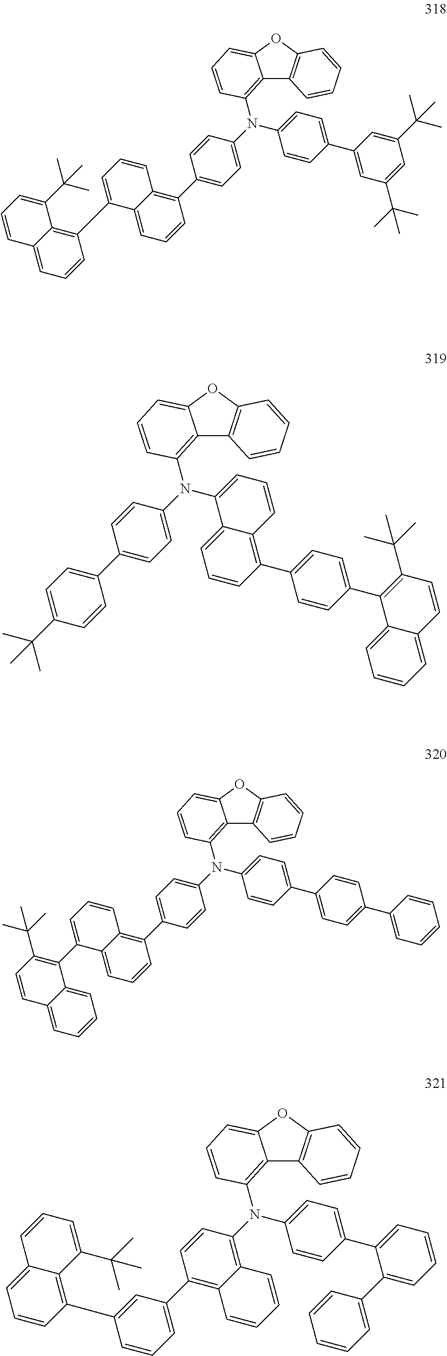

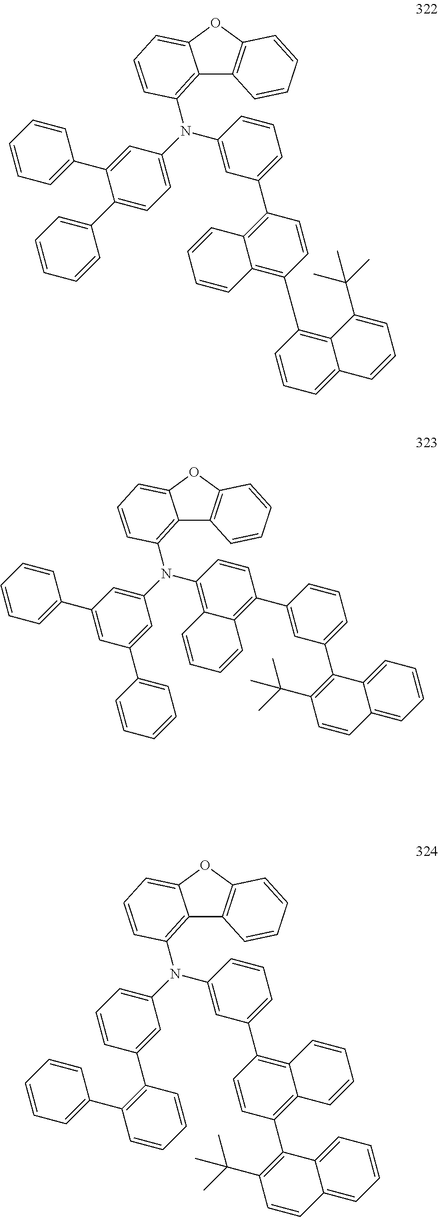 [SMILES: CC(C)(C)c1cccc2cccc(-c3ccc(-c4cccc(N(c5ccc(-c6ccccc6)c(-c6ccccc6)c5)c5cccc6oc7ccccc7c56)c4)c4ccccc34)c12 ; CC(C)(C)c1ccc2ccccc2c1-c1cccc(-c2ccc(N(c3cc(-c4ccccc4)cc(-c4ccccc4)c3)c3cccc4oc5ccccc5c34)c3ccccc23)c1 ; CC(C)(C)c1ccc2ccccc2c1-c1ccc(-c2cccc(N(c3cccc(-c4ccccc4-c4ccccc4)c3)c3cccc4oc5ccccc5c34)c2)c2ccccc12]

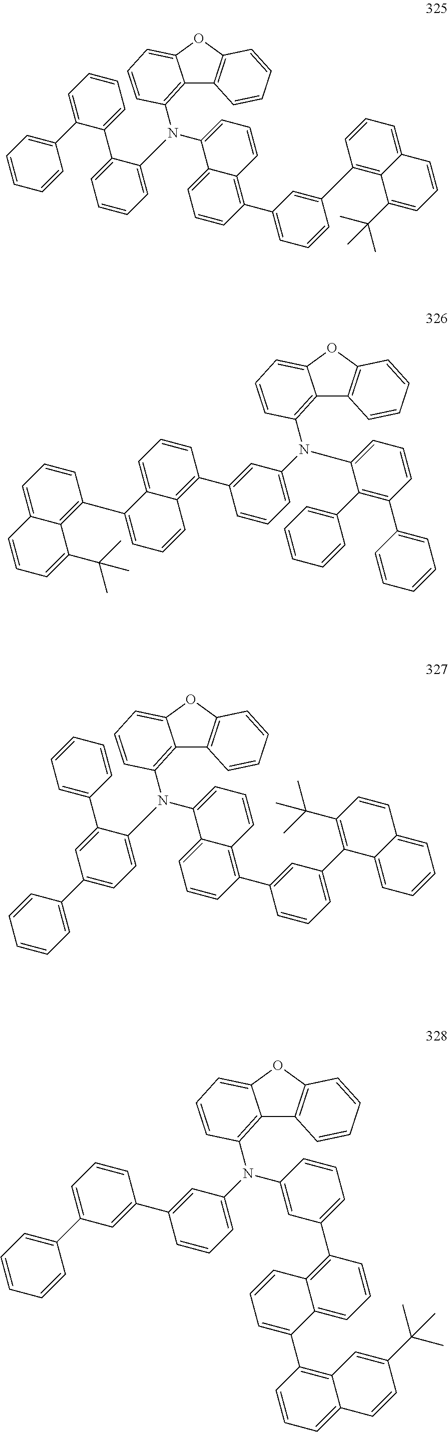 [SMILES: CC(C)(C)c1cccc2cccc(-c3cccc(-c4cccc5c(N(c6ccccc6-c6ccccc6-c6ccccc6)c6cccc7oc8ccccc8c67)cccc45)c3)c12 ; CC(C)(C)c1cccc2cccc(-c3cccc4c(-c5cccc(N(c6cccc(-c7ccccc7)c6-c6ccccc6)c6cccc7oc8ccccc8c67)c5)cccc34)c12 ; CC(C)(C)c1ccc2ccccc2c1-c1cccc(-c2cccc3c(N(c4ccc(-c5ccccc5)cc4-c4ccccc4)c4cccc5oc6ccccc6c45)cccc23)c1 ; CC(C)(C)c1ccc2cccc(-c3cccc4c(-c5cccc(N(c6cccc(-c7cccc(-c8ccccc8)c7)c6)c6cccc7oc8ccccc8c67)c5)cccc34)c2c1]

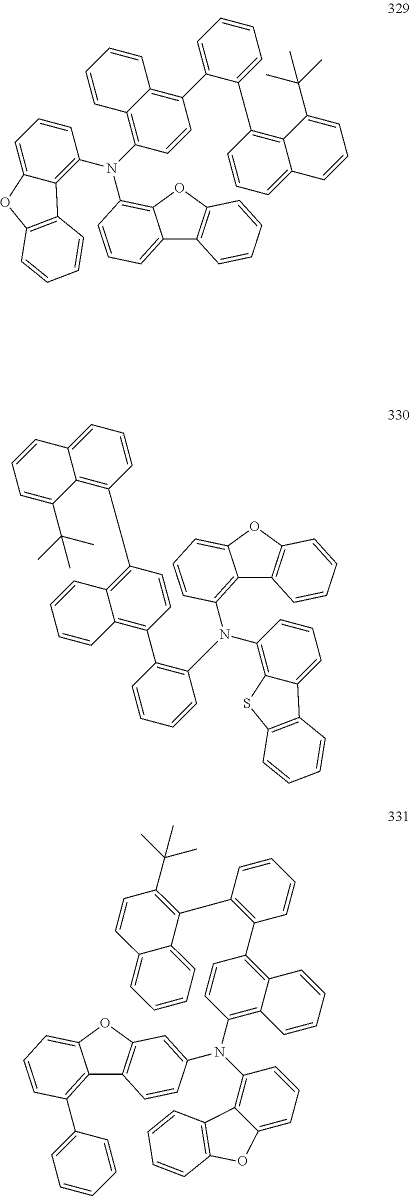 [SMILES: CC(C)(C)c1cccc2cccc(-c3ccccc3-c3ccc(N(c4cccc5c4oc4ccccc45)c4cccc5oc6ccccc6c45)c4ccccc34)c12 ; CC(C)(C)c1cccc2cccc(-c3ccc(-c4ccccc4N(c4cccc5c4sc4ccccc45)c4cccc5oc6ccccc6c45)c4ccccc34)c12 ; CC(C)(C)c1ccc2ccccc2c1-c1ccccc1-c1ccc(N(c2ccc3c(c2)oc2cccc(-c4ccccc4)c23)c2cccc3oc4ccccc4c23)c2ccccc12]

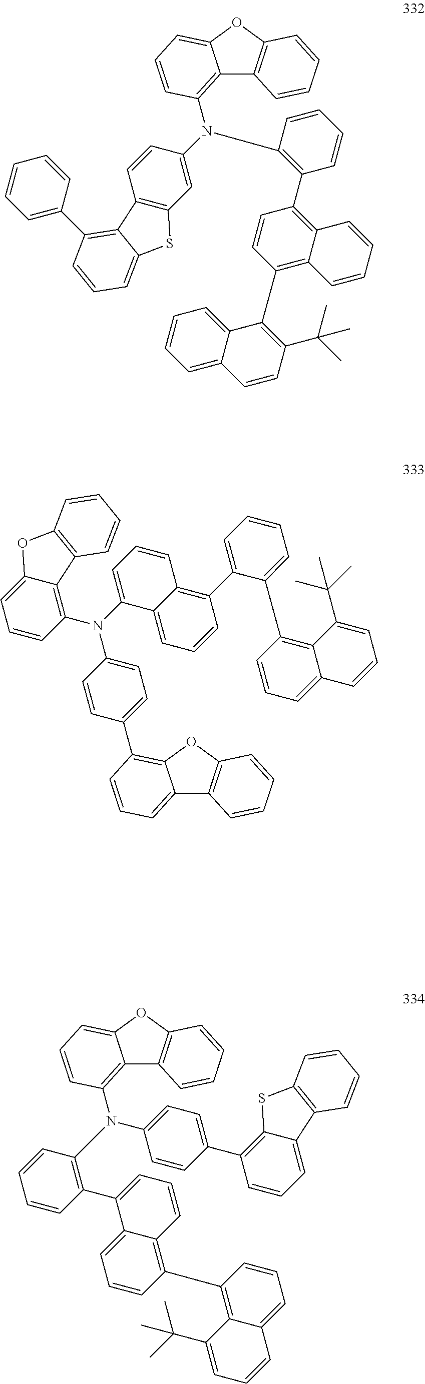 [SMILES: CC(C)(C)c1ccc2ccccc2c1-c1ccc(-c2ccccc2N(c2ccc3c(c2)sc2cccc(-c4ccccc4)c23)c2cccc3oc4ccccc4c23)c2ccccc12 ; CC(C)(C)c1cccc2cccc(-c3ccccc3-c3cccc4c(N(c5ccc(-c6cccc7c6oc6ccccc67)cc5)c5cccc6oc7ccccc7c56)cccc34)c12 ; CC(C)(C)c1cccc2cccc(-c3cccc4c(-c5ccccc5N(c5ccc(-c6cccc7c6sc6ccccc67)cc5)c5cccc6oc7ccccc7c56)cccc34)c12]

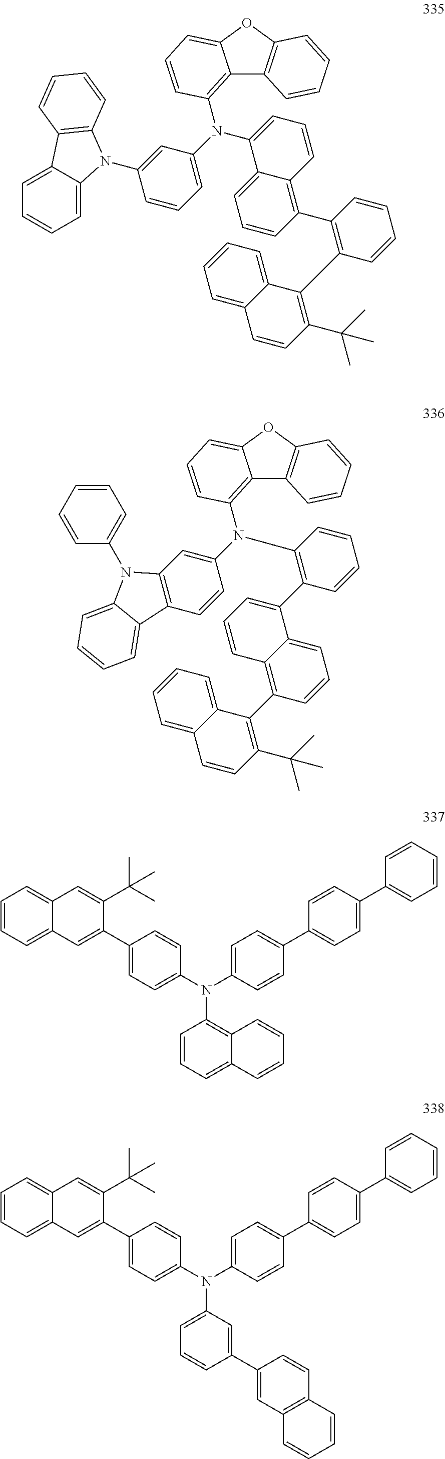 [SMILES: CC(C)(C)c1ccc2ccccc2c1-c1ccccc1-c1cccc2c(N(c3cccc(-n4c5ccccc5c5ccccc54)c3)c3cccc4oc5ccccc5c34)cccc12 ; CC(C)(C)c1ccc2ccccc2c1-c1cccc2c(-c3ccccc3N(c3ccc4c5ccccc5n(-c5ccccc5)c4c3)c3cccc4oc5ccccc5c34)cccc12 ; CC(C)(C)c1cc2ccccc2cc1-c1ccc(N(c2ccc(-c3ccc(-c4ccccc4)cc3)cc2)c2cccc3ccccc23)cc1 ; CC(C)(C)c1cc2ccccc2cc1-c1ccc(N(c2ccc(-c3ccc(-c4ccccc4)cc3)cc2)c2cccc(-c3ccc4ccccc4c3)c2)cc1]

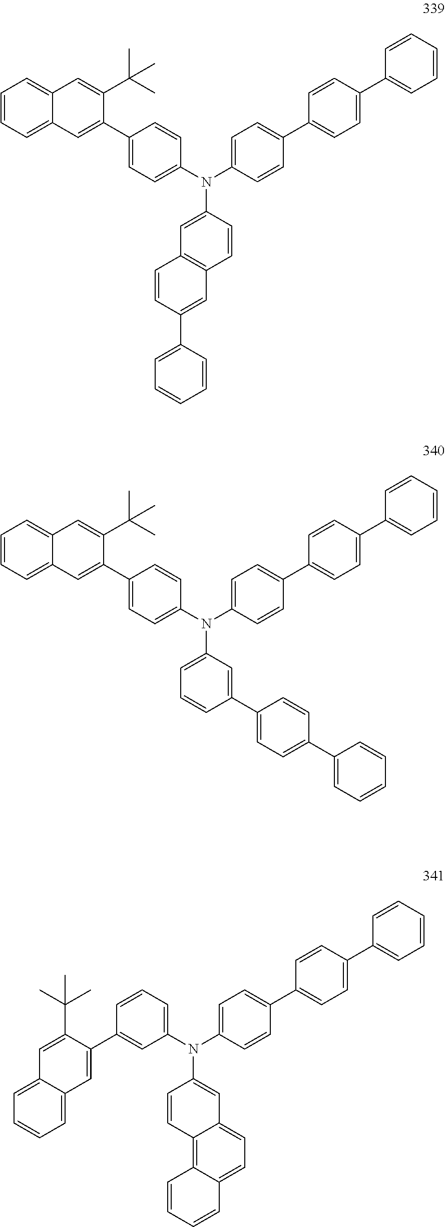 [SMILES: CC(C)(C)c1cc2ccccc2cc1-c1ccc(N(c2ccc(-c3ccc(-c4ccccc4)cc3)cc2)c2ccc3cc(-c4ccccc4)ccc3c2)cc1 ; CC(C)(C)c1cc2ccccc2cc1-c1ccc(N(c2ccc(-c3ccc(-c4ccccc4)cc3)cc2)c2cccc(-c3ccc(-c4ccccc4)cc3)c2)cc1 ; CC(C)(C)c1cc2ccccc2cc1-c1cccc(N(c2ccc(-c3ccc(-c4ccccc4)cc3)cc2)c2ccc3c(ccc4ccccc43)c2)c1]

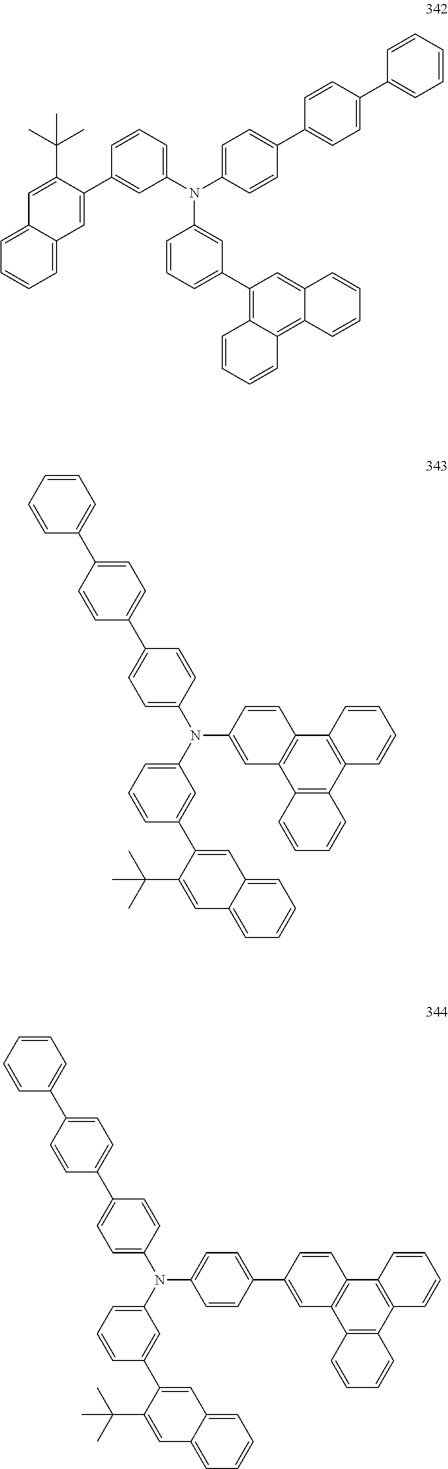 [SMILES: CC(C)(C)c1cc2ccccc2cc1-c1cccc(N(c2ccc(-c3ccc(-c4ccccc4)cc3)cc2)c2cccc(-c3cc4ccccc4c4ccccc34)c2)c1 ; CC(C)(C)c1cc2ccccc2cc1-c1cccc(N(c2ccc(-c3ccc(-c4ccccc4)cc3)cc2)c2ccc3c4ccccc4c4ccccc4c3c2)c1 ; CC(C)(C)c1cc2ccccc2cc1-c1cccc(N(c2ccc(-c3ccc(-c4ccccc4)cc3)cc2)c2ccc(-c3ccc4c5ccccc5c5ccccc5c4c3)cc2)c1]

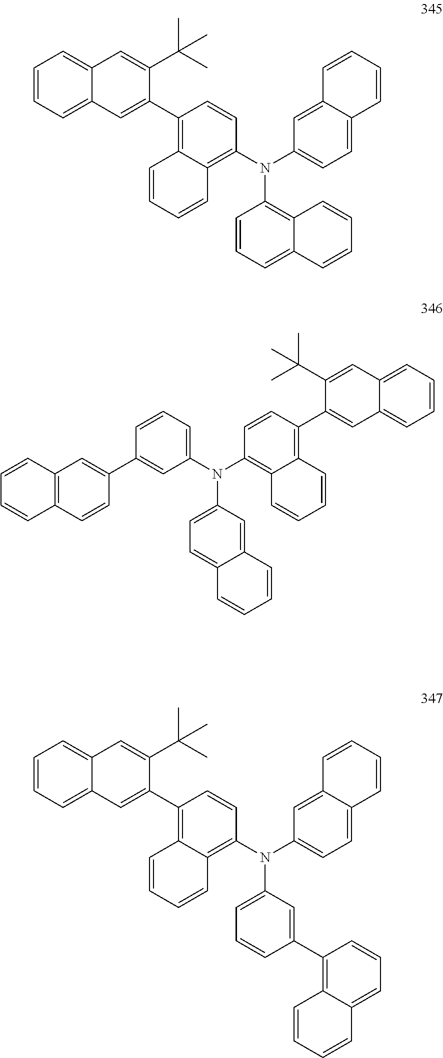 [SMILES: CC(C)(C)c1cc2ccccc2cc1-c1ccc(N(c2ccc3ccccc3c2)c2cccc3ccccc23)c2ccccc12 ; CC(C)(C)c1cc2ccccc2cc1-c1ccc(N(c2cccc(-c3ccc4ccccc4c3)c2)c2ccc3ccccc3c2)c2ccccc12 ; CC(C)(C)c1cc2ccccc2cc1-c1ccc(N(c2cccc(-c3cccc4ccccc34)c2)c2ccc3ccccc3c2)c2ccccc12]

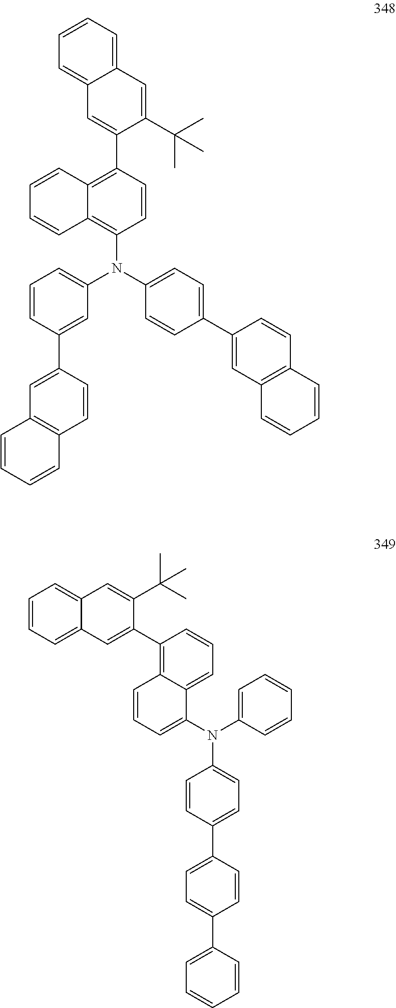 [SMILES: CC(C)(C)c1cc2ccccc2cc1-c1ccc(N(c2ccc(-c3ccc4ccccc4c3)cc2)c2cccc(-c3ccc4ccccc4c3)c2)c2ccccc12 ; CC(C)(C)c1cc2ccccc2cc1-c1cccc2c(N(c3ccccc3)c3ccc(-c4ccc(-c5ccccc5)cc4)cc3)cccc12]

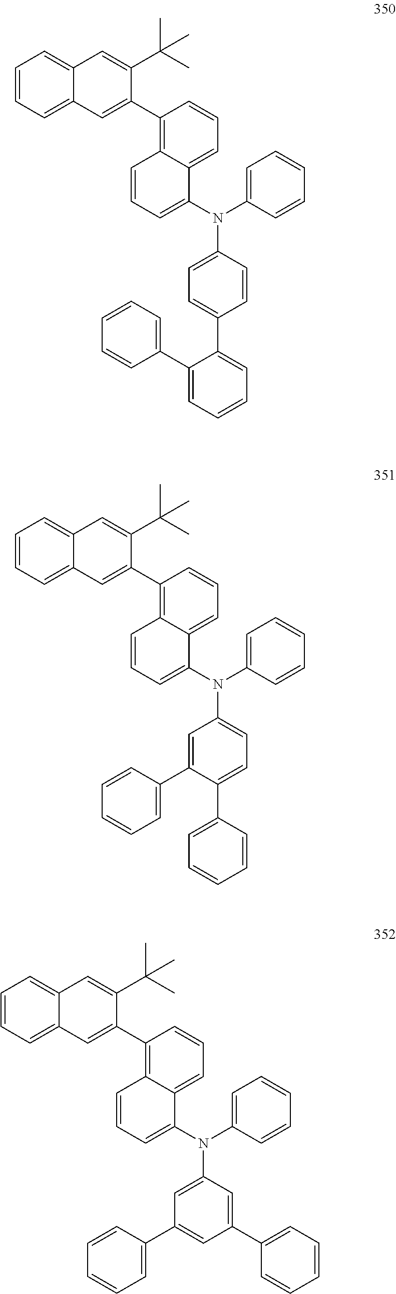 [SMILES: CC(C)(C)c1cc2ccccc2cc1-c1cccc2c(N(c3ccccc3)c3ccc(-c4ccccc4-c4ccccc4)cc3)cccc12 ; CC(C)(C)c1cc2ccccc2cc1-c1cccc2c(N(c3ccccc3)c3ccc(-c4ccccc4)c(-c4ccccc4)c3)cccc12 ; CC(C)(C)c1cc2ccccc2cc1-c1cccc2c(N(c3ccccc3)c3cc(-c4ccccc4)cc(-c4ccccc4)c3)cccc12]

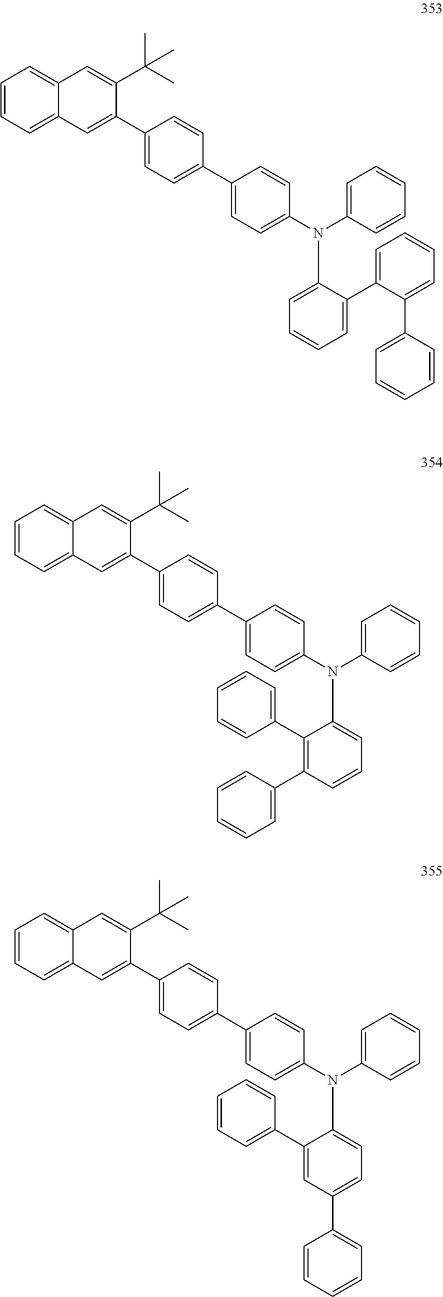 [SMILES: CC(C)(C)c1cc2ccccc2cc1-c1ccc(-c2ccc(N(c3ccccc3)c3ccccc3-c3ccccc3-c3ccccc3)cc2)cc1 ; CC(C)(C)c1cc2ccccc2cc1-c1ccc(-c2ccc(N(c3ccccc3)c3cccc(-c4ccccc4)c3-c3ccccc3)cc2)cc1 ; CC(C)(C)c1cc2ccccc2cc1-c1ccc(-c2ccc(N(c3ccccc3)c3ccc(-c4ccccc4)cc3-c3ccccc3)cc2)cc1]

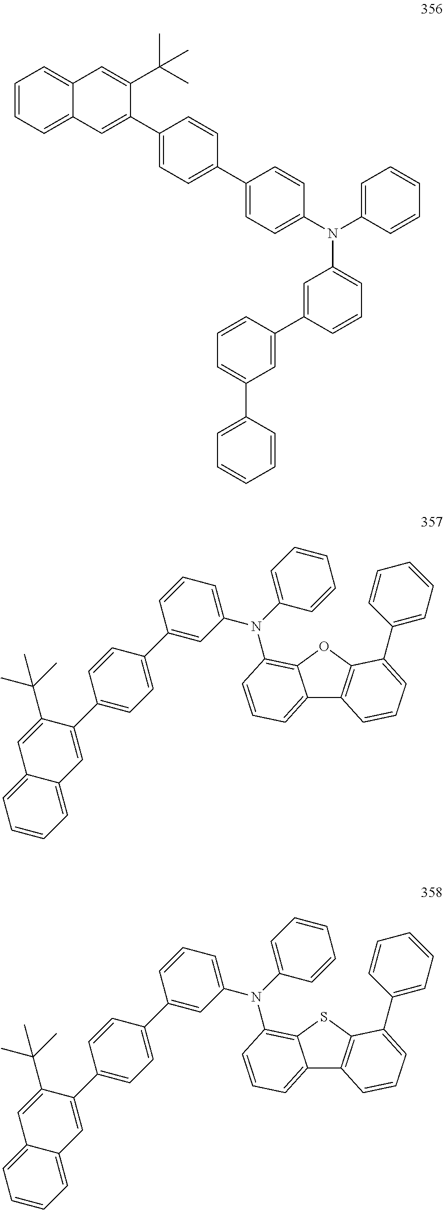 [SMILES: CC(C)(C)c1cc2ccccc2cc1-c1ccc(-c2ccc(N(c3ccccc3)c3cccc(-c4cccc(-c5ccccc5)c4)c3)cc2)cc1 ; CC(C)(C)c1cc2ccccc2cc1-c1ccc(-c2cccc(N(c3ccccc3)c3cccc4c3oc3c(-c5ccccc5)cccc34)c2)cc1 ; CC(C)(C)c1cc2ccccc2cc1-c1ccc(-c2cccc(N(c3ccccc3)c3cccc4c3sc3c(-c5ccccc5)cccc34)c2)cc1]

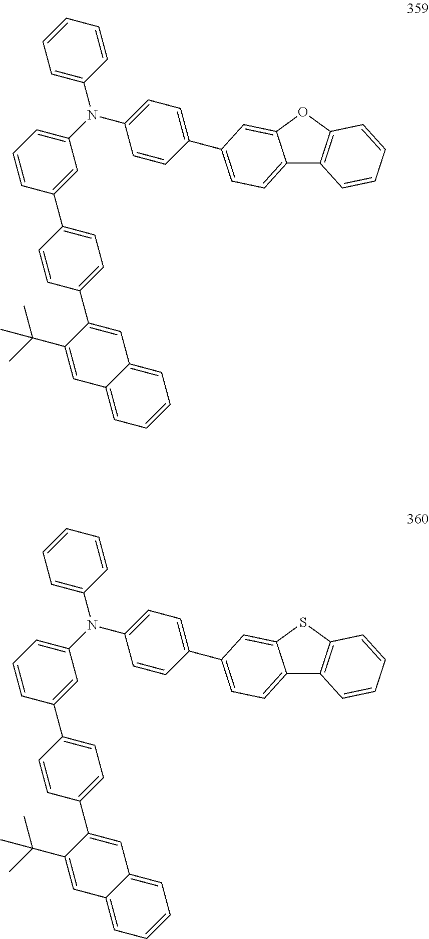 [SMILES: CC(C)(C)c1cc2ccccc2cc1-c1ccc(-c2cccc(N(c3ccccc3)c3ccc(-c4ccc5c(c4)oc4ccccc45)cc3)c2)cc1 ; CC(C)(C)c1cc2ccccc2cc1-c1ccc(-c2cccc(N(c3ccccc3)c3ccc(-c4ccc5c(c4)sc4ccccc45)cc3)c2)cc1]

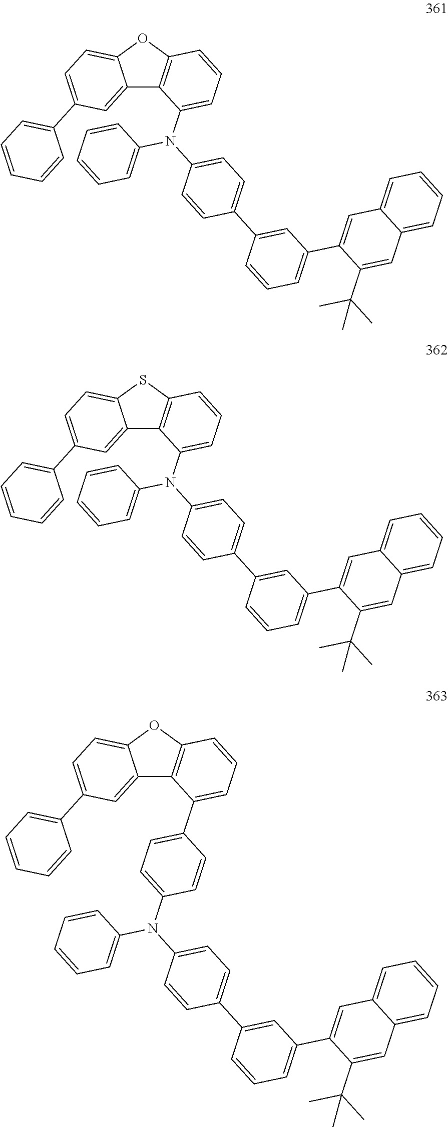 [SMILES: CC(C)(C)c1cc2ccccc2cc1-c1cccc(-c2ccc(N(c3ccccc3)c3cccc4oc5ccc(-c6ccccc6)cc5c34)cc2)c1 ; CC(C)(C)c1cc2ccccc2cc1-c1cccc(-c2ccc(N(c3ccccc3)c3cccc4sc5ccc(-c6ccccc6)cc5c34)cc2)c1 ; CC(C)(C)c1cc2ccccc2cc1-c1cccc(-c2ccc(N(c3ccccc3)c3ccc(-c4cccc5oc6ccc(-c7ccccc7)cc6c45)cc3)cc2)c1]

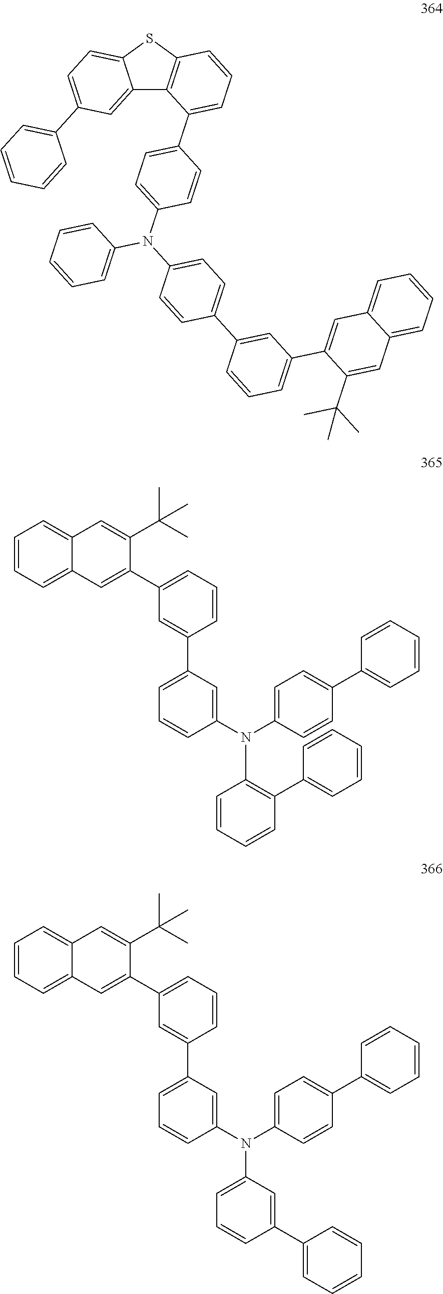 [SMILES: CC(C)(C)c1cc2ccccc2cc1-c1cccc(-c2ccc(N(c3ccccc3)c3ccc(-c4cccc5sc6ccc(-c7ccccc7)cc6c45)cc3)cc2)c1 ; CC(C)(C)c1cc2ccccc2cc1-c1cccc(-c2cccc(N(c3ccc(-c4ccccc4)cc3)c3ccccc3-c3ccccc3)c2)c1 ; CC(C)(C)c1cc2ccccc2cc1-c1cccc(-c2cccc(N(c3ccc(-c4ccccc4)cc3)c3cccc(-c4ccccc4)c3)c2)c1]

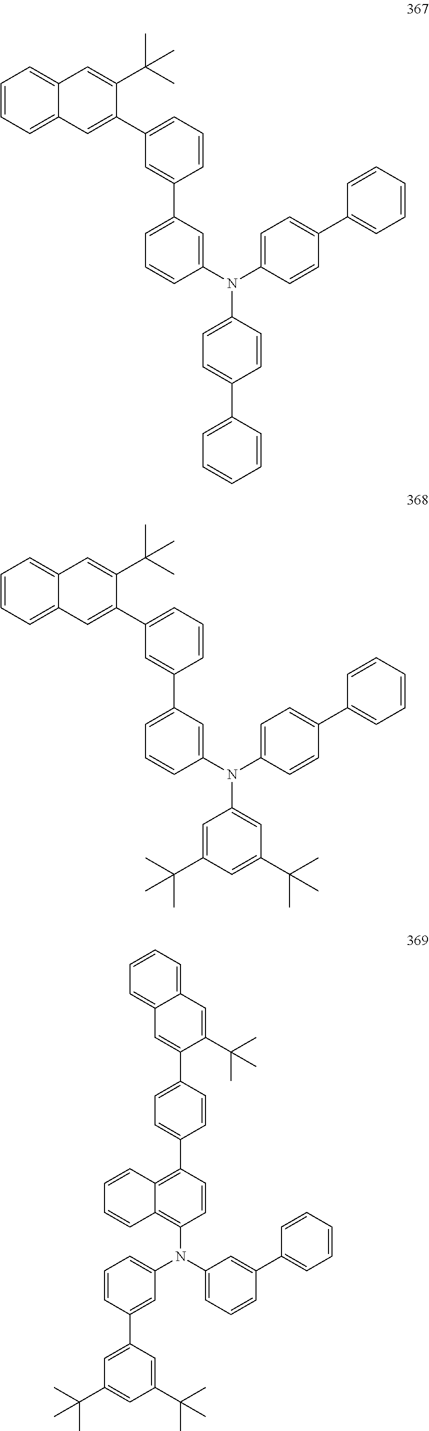

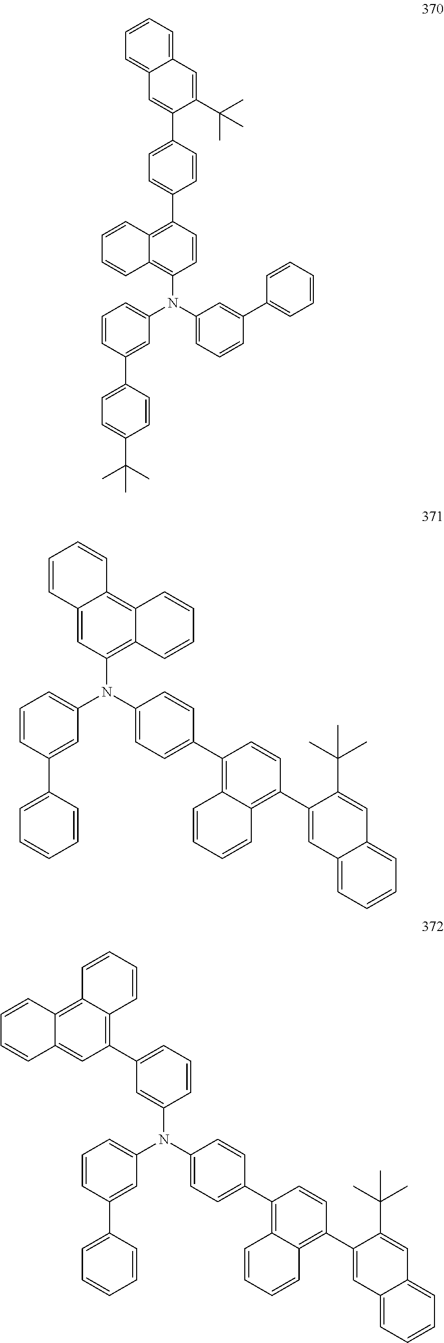 [SMILES: CC(C)(C)c1ccc(-c2cccc(N(c3cccc(-c4ccccc4)c3)c3ccc(-c4ccc(-c5cc6ccccc6cc5C(C)(C)C)cc4)c4ccccc34)c2)cc1 ; CC(C)(C)c1cc2ccccc2cc1-c1ccc(-c2ccc(N(c3cccc(-c4ccccc4)c3)c3cc4ccccc4c4ccccc34)cc2)c2ccccc12 ; CC(C)(C)c1cc2ccccc2cc1-c1ccc(-c2ccc(N(c3cccc(-c4ccccc4)c3)c3cccc(-c4cc5ccccc5c5ccccc45)c3)cc2)c2ccccc12]

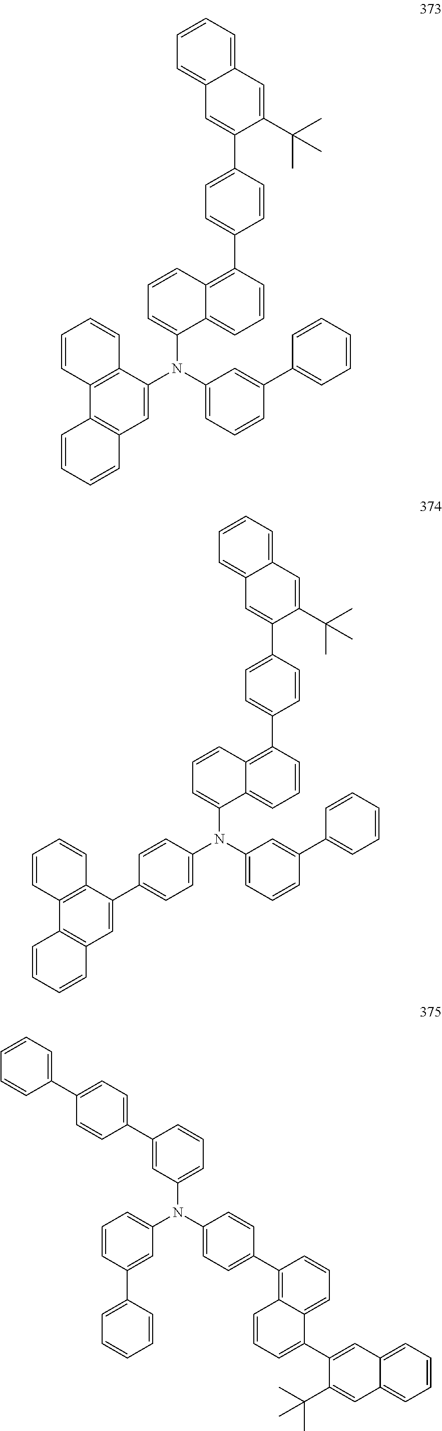 [SMILES: CC(C)(C)c1cc2ccccc2cc1-c1ccc(-c2cccc3c(N(c4cccc(-c5ccccc5)c4)c4cc5ccccc5c5ccccc45)cccc23)cc1 ; CC(C)(C)c1cc2ccccc2cc1-c1ccc(-c2cccc3c(N(c4ccc(-c5cc6ccccc6c6ccccc56)cc4)c4cccc(-c5ccccc5)c4)cccc23)cc1 ; CC(C)(C)c1cc2ccccc2cc1-c1cccc2c(-c3ccc(N(c4cccc(-c5ccccc5)c4)c4cccc(-c5ccc(-c6ccccc6)cc5)c4)cc3)cccc12]

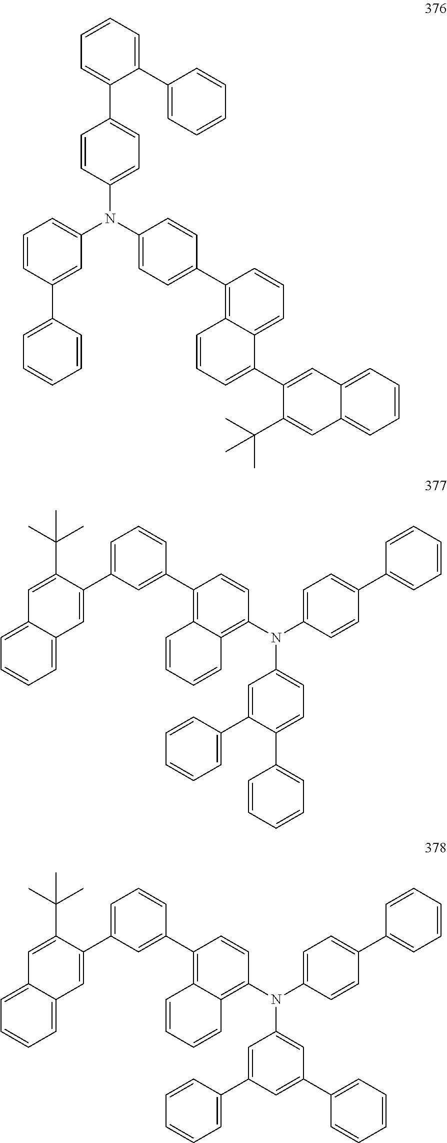 [SMILES: CC(C)(C)c1cc2ccccc2cc1-c1cccc2c(-c3ccc(N(c4ccc(-c5ccccc5-c5ccccc5)cc4)c4cccc(-c5ccccc5)c4)cc3)cccc12 ; CC(C)(C)c1cc2ccccc2cc1-c1cccc(-c2ccc(N(c3ccc(-c4ccccc4)cc3)c3ccc(-c4ccccc4)c(-c4ccccc4)c3)c3ccccc23)c1 ; CC(C)(C)c1cc2ccccc2cc1-c1cccc(-c2ccc(N(c3ccc(-c4ccccc4)cc3)c3cc(-c4ccccc4)cc(-c4ccccc4)c3)c3ccccc23)c1]

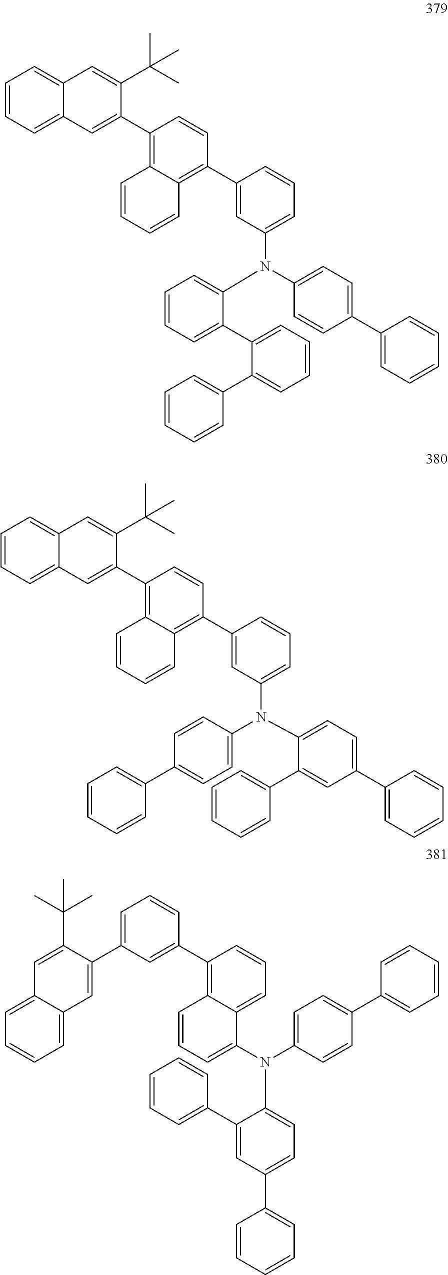 [SMILES: CC(C)(C)c1cc2ccccc2cc1-c1ccc(-c2cccc(N(c3ccc(-c4ccccc4)cc3)c3ccccc3-c3ccccc3-c3ccccc3)c2)c2ccccc12 ; CC(C)(C)c1cc2ccccc2cc1-c1ccc(-c2cccc(N(c3ccc(-c4ccccc4)cc3)c3ccc(-c4ccccc4)cc3-c3ccccc3)c2)c2ccccc12 ; CC(C)(C)c1cc2ccccc2cc1-c1cccc(-c2cccc3c(N(c4ccc(-c5ccccc5)cc4)c4ccc(-c5ccccc5)cc4-c4ccccc4)cccc23)c1]

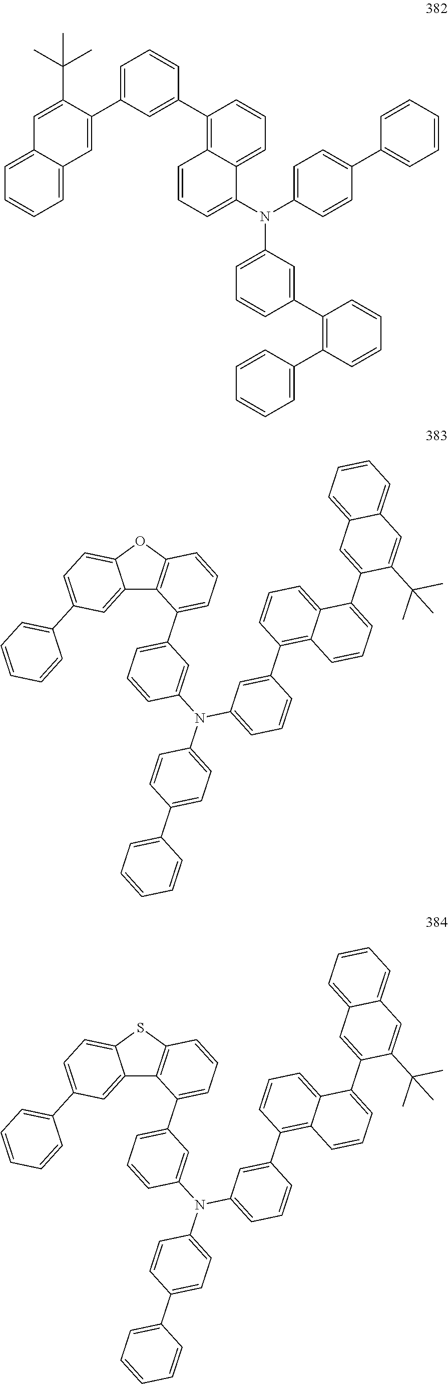 [SMILES: CC(C)(C)c1cc2ccccc2cc1-c1cccc(-c2cccc3c(N(c4ccc(-c5ccccc5)cc4)c4cccc(-c5ccccc5-c5ccccc5)c4)cccc23)c1 ; CC(C)(C)c1cc2ccccc2cc1-c1cccc2c(-c3cccc(N(c4ccc(-c5ccccc5)cc4)c4cccc(-c5cccc6oc7ccc(-c8ccccc8)cc7c56)c4)c3)cccc12 ; CC(C)(C)c1cc2ccccc2cc1-c1cccc2c(-c3cccc(N(c4ccc(-c5ccccc5)cc4)c4cccc(-c5cccc6sc7ccc(-c8ccccc8)cc7c56)c4)c3)cccc12]

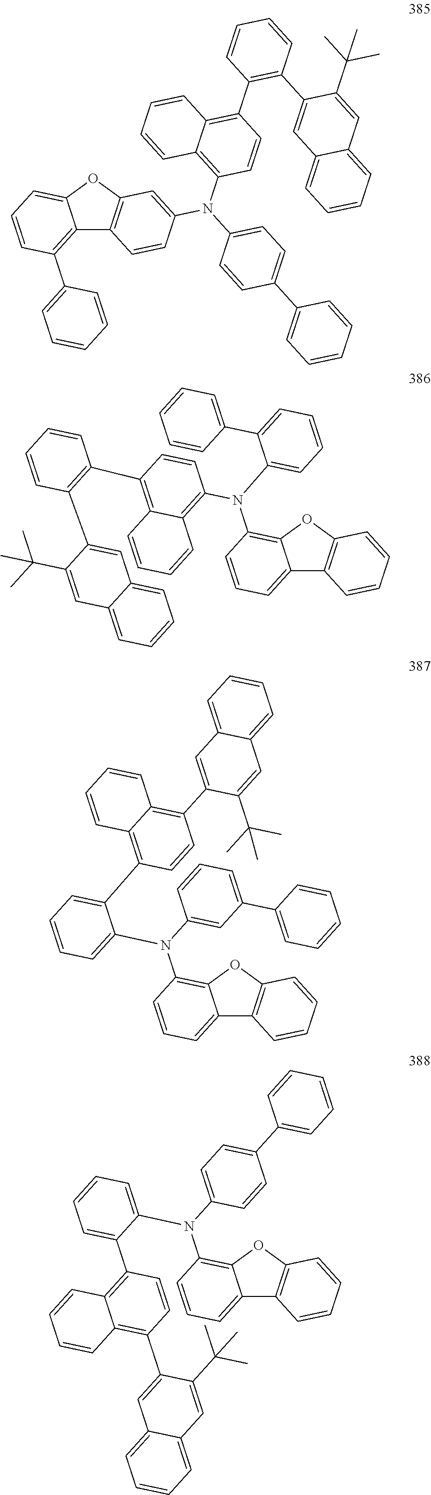

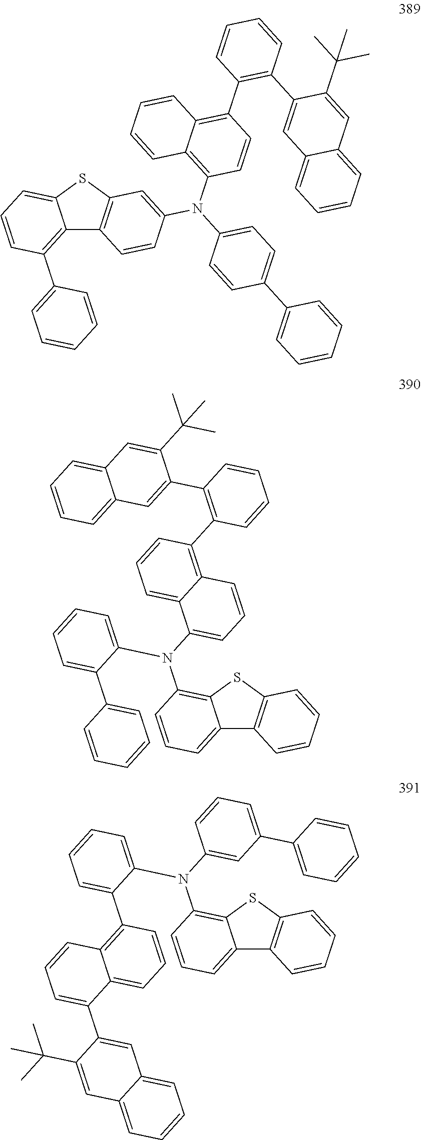

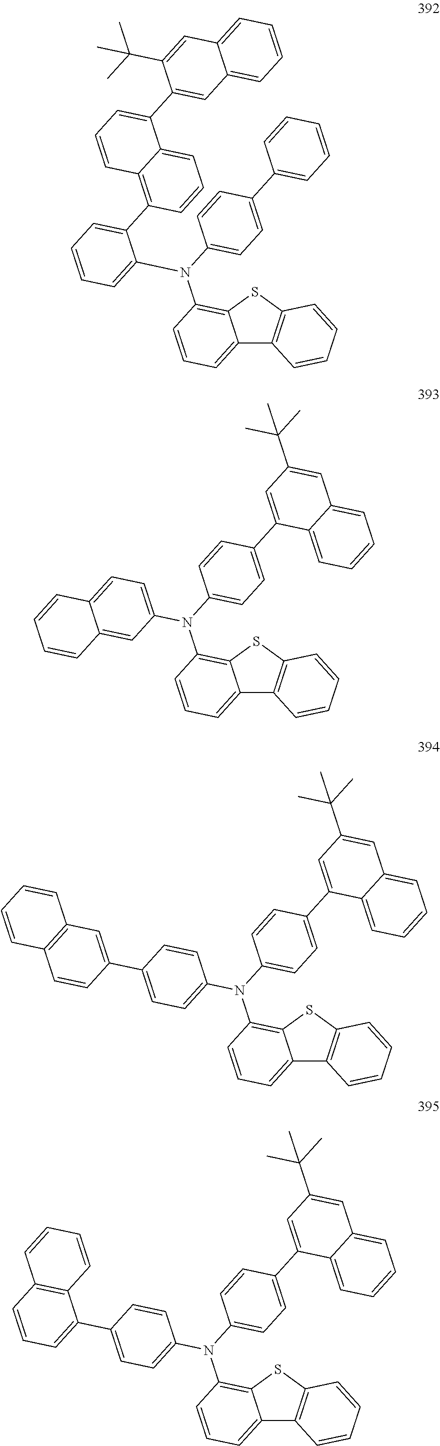

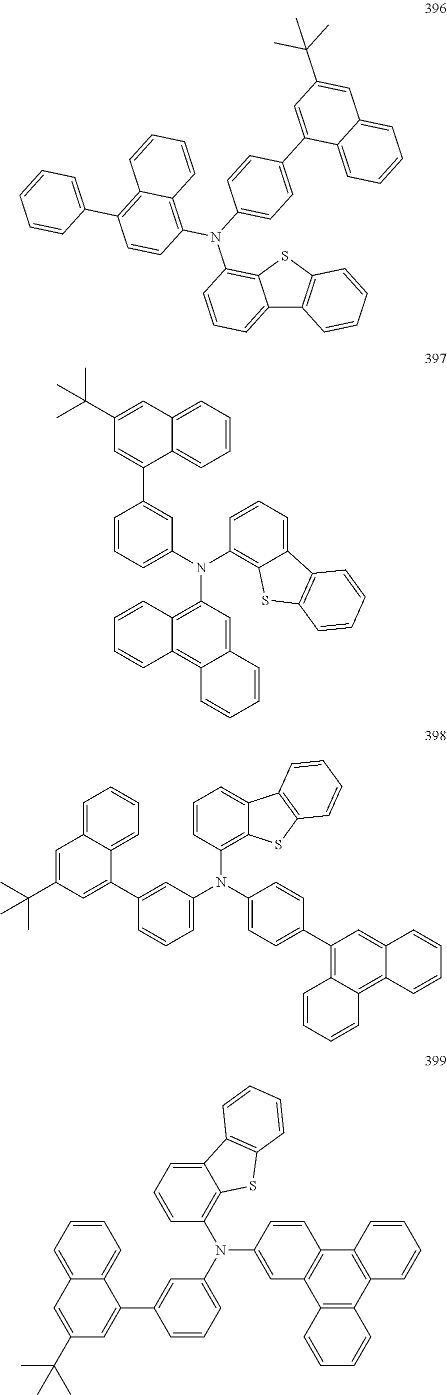 [SMILES: CC(C)(C)c1cc(-c2ccc(N(c3ccc(-c4ccccc4)c4ccccc34)c3cccc4c3sc3ccccc34)cc2)c2ccccc2c1 ; CC(C)(C)c1cc(-c2cccc(N(c3cc4ccccc4c4ccccc34)c3cccc4c3sc3ccccc34)c2)c2ccccc2c1 ; CC(C)(C)c1cc(-c2cccc(N(c3ccc(-c4cc5ccccc5c5ccccc45)cc3)c3cccc4c3sc3ccccc34)c2)c2ccccc2c1 ; CC(C)(C)c1cc(-c2cccc(N(c3ccc4c5ccccc5c5ccccc5c4c3)c3cccc4c3sc3ccccc34)c2)c2ccccc2c1]

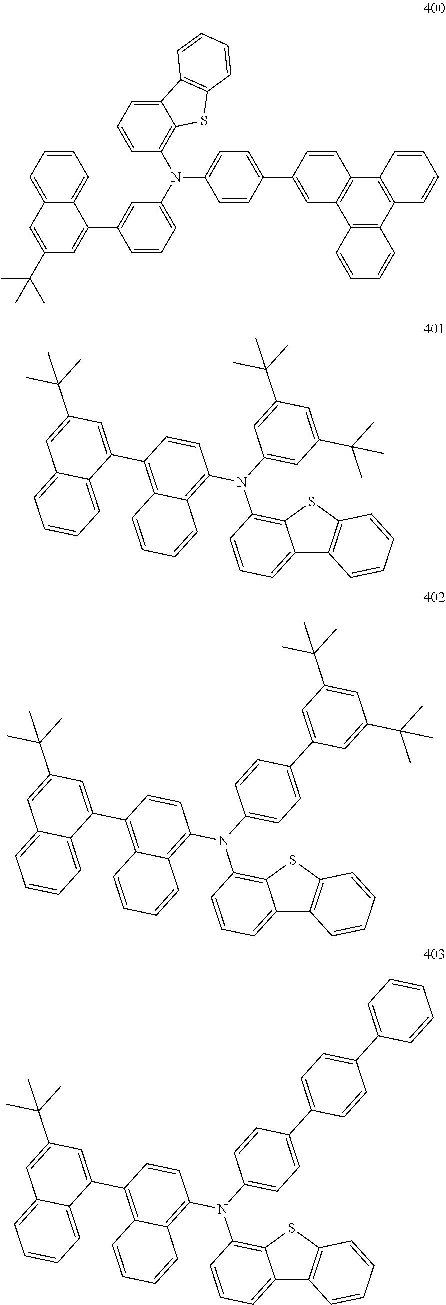

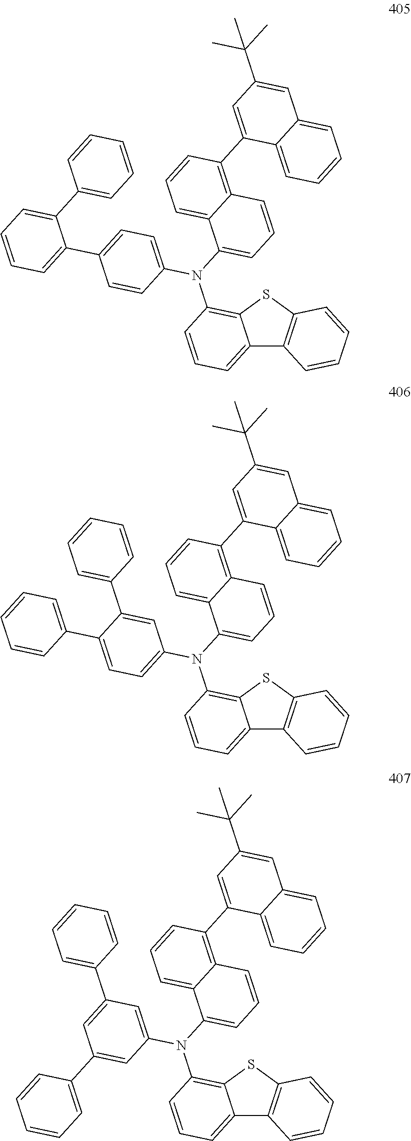

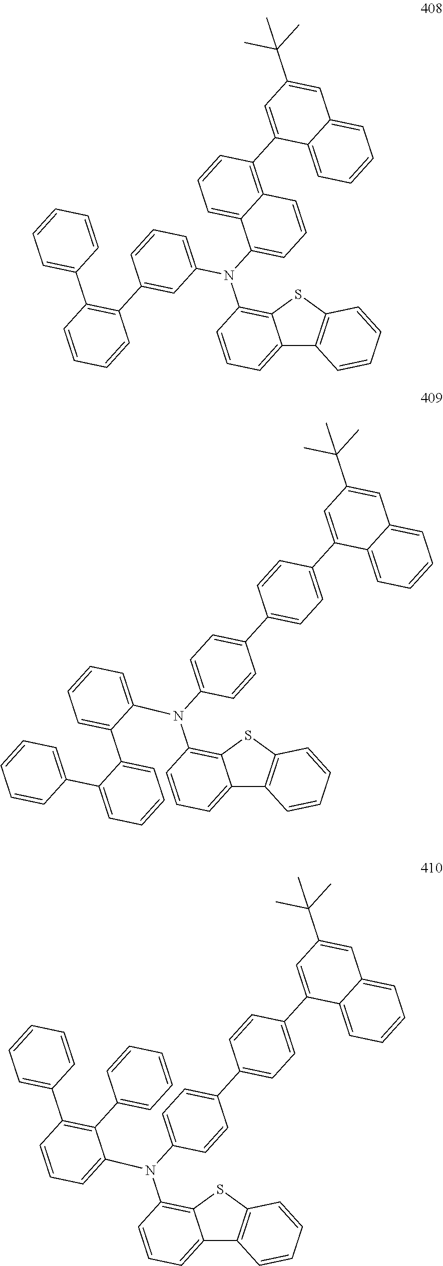 [SMILES: CC(C)(C)c1cc(-c2cccc3c(N(c4cccc(-c5ccccc5-c5ccccc5)c4)c4cccc5c4sc4ccccc45)cccc23)c2ccccc2c1 ; CC(C)(C)c1cc(-c2ccc(-c3ccc(N(c4ccccc4-c4ccccc4-c4ccccc4)c4cccc5c4sc4ccccc45)cc3)cc2)c2ccccc2c1 ; CC(C)(C)c1cc(-c2ccc(-c3ccc(N(c4cccc(-c5ccccc5)c4-c4ccccc4)c4cccc5c4sc4ccccc45)cc3)cc2)c2ccccc2c1]

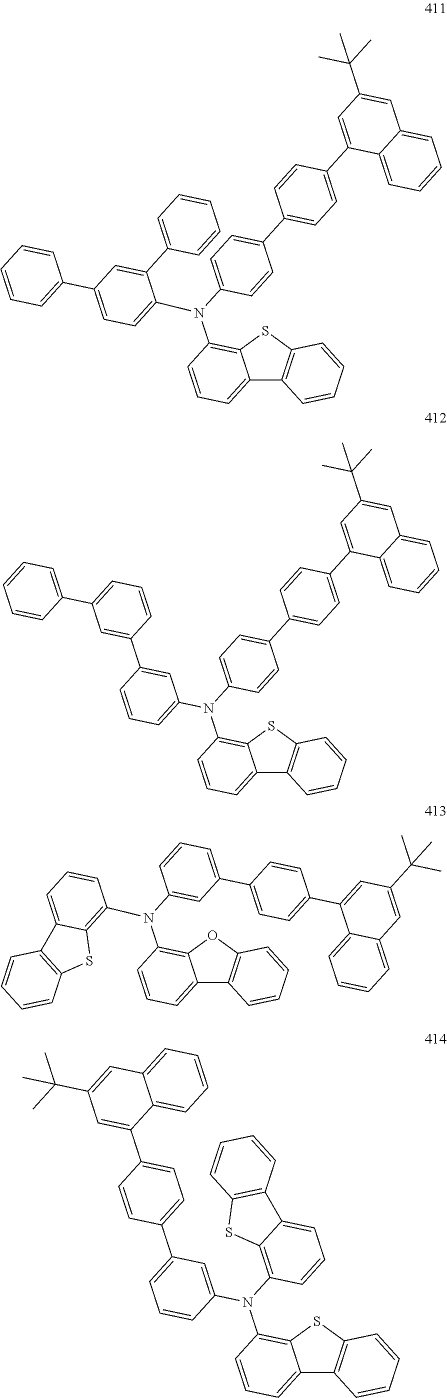

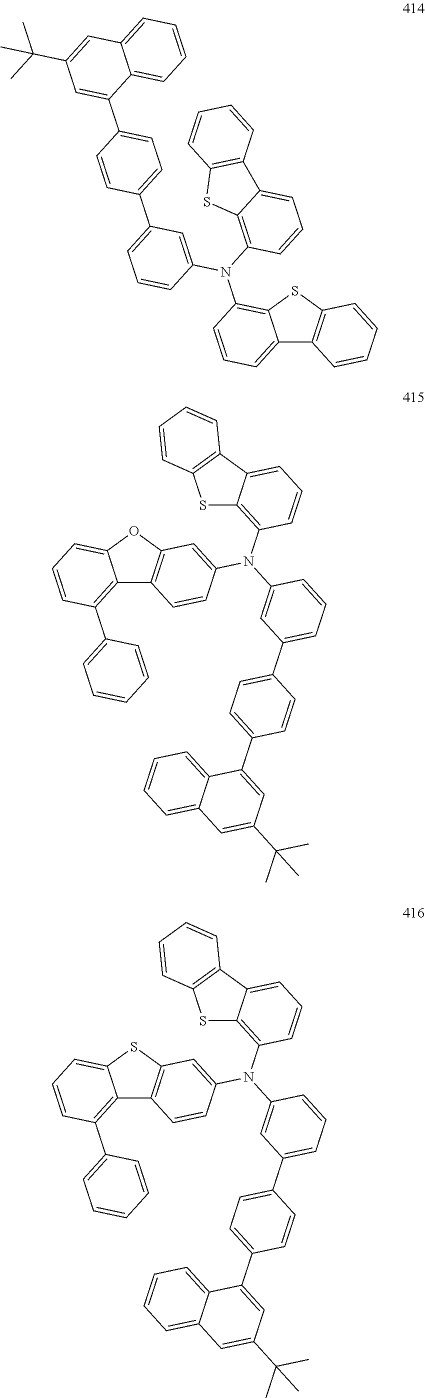 [SMILES: CC(C)(C)c1cc(-c2ccc(-c3cccc(N(c4cccc5c4sc4ccccc45)c4cccc5c4sc4ccccc45)c3)cc2)c2ccccc2c1 ; CC(C)(C)c1cc(-c2ccc(-c3cccc(N(c4ccc5c(c4)oc4cccc(-c6ccccc6)c45)c4cccc5c4sc4ccccc45)c3)cc2)c2ccccc2c1 ; CC(C)(C)c1cc(-c2ccc(-c3cccc(N(c4ccc5c(c4)sc4cccc(-c6ccccc6)c45)c4cccc5c4sc4ccccc45)c3)cc2)c2ccccc2c1]

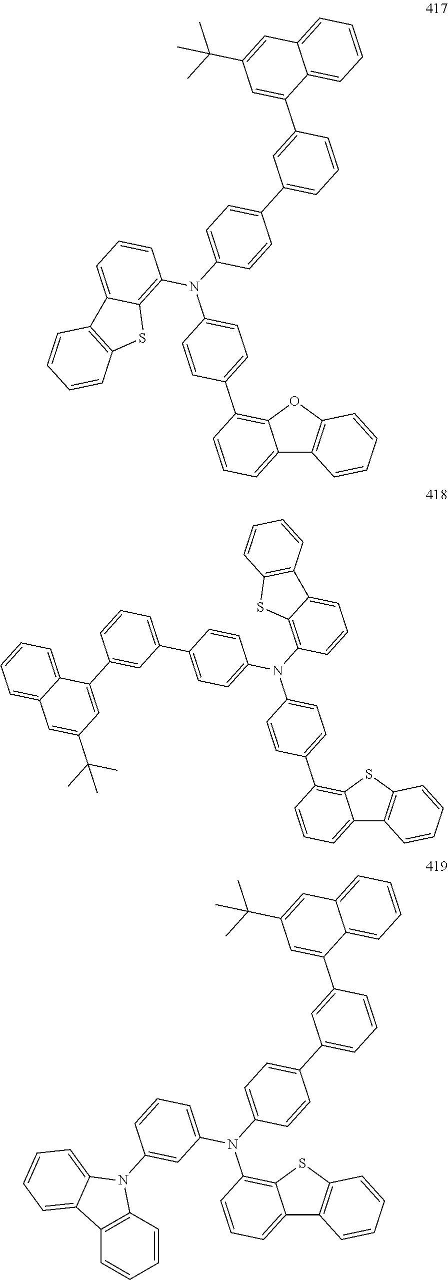

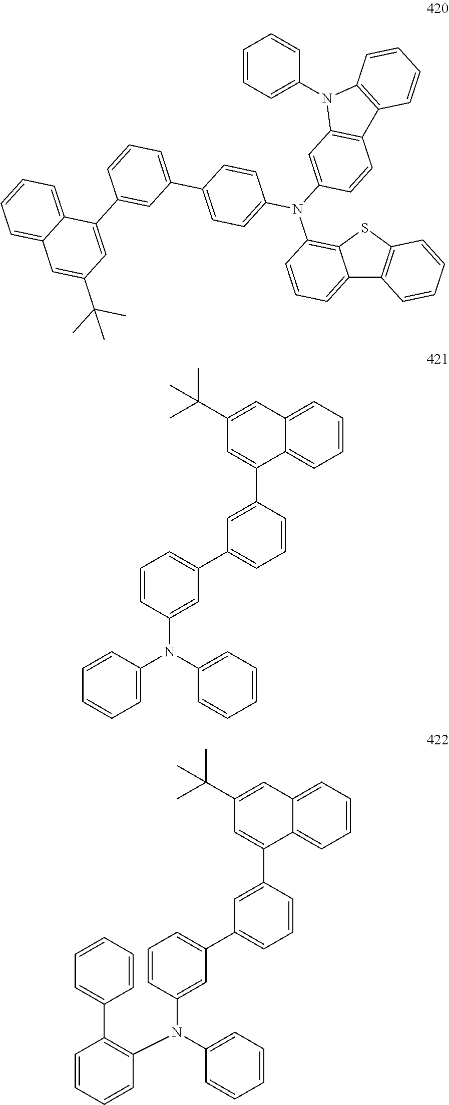

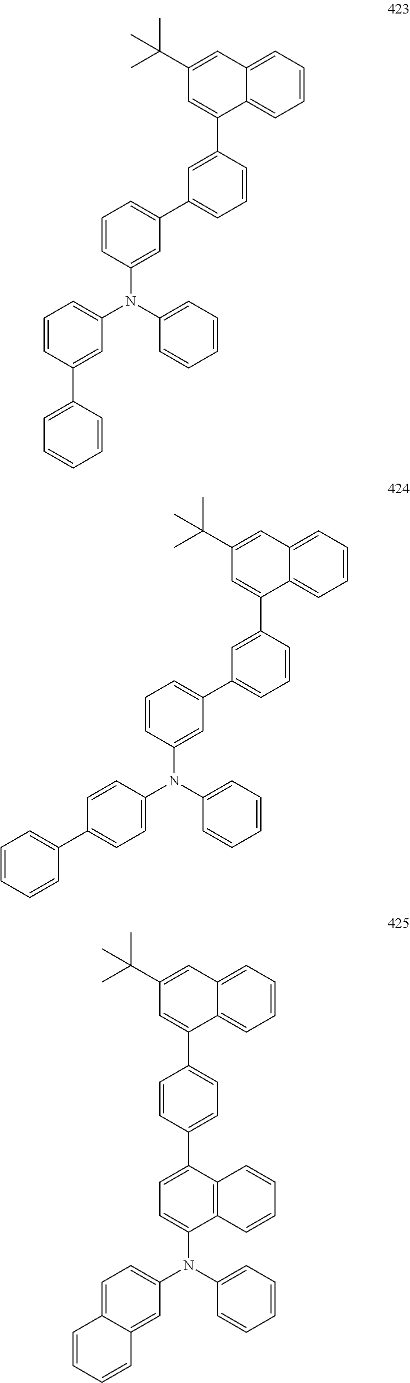 [SMILES: CC(C)(C)c1cc(-c2cccc(-c3cccc(N(c4ccccc4)c4cccc(-c5ccccc5)c4)c3)c2)c2ccccc2c1 ; CC(C)(C)c1cc(-c2cccc(-c3cccc(N(c4ccccc4)c4ccc(-c5ccccc5)cc4)c3)c2)c2ccccc2c1 ; CC(C)(C)c1cc(-c2ccc(-c3ccc(N(c4ccccc4)c4ccc5ccccc5c4)c4ccccc34)cc2)c2ccccc2c1]

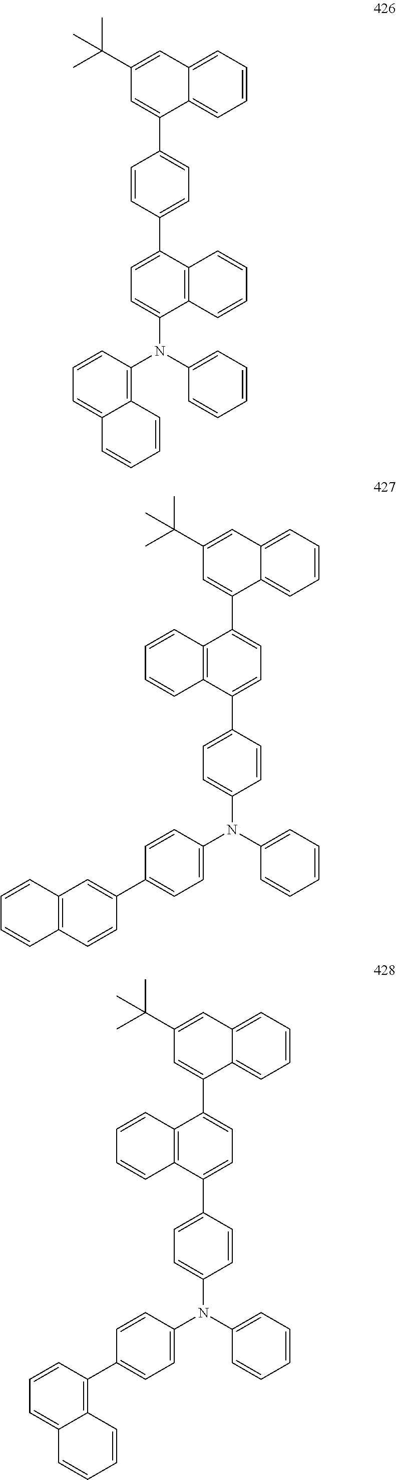 [SMILES: CC(C)(C)c1cc(-c2ccc(-c3ccc(N(c4ccccc4)c4cccc5ccccc45)c4ccccc34)cc2)c2ccccc2c1 ; CC(C)(C)c1cc(-c2ccc(-c3ccc(N(c4ccccc4)c4ccc(-c5ccc6ccccc6c5)cc4)cc3)c3ccccc23)c2ccccc2c1 ; CC(C)(C)c1cc(-c2ccc(-c3ccc(N(c4ccccc4)c4ccc(-c5cccc6ccccc56)cc4)cc3)c3ccccc23)c2ccccc2c1]

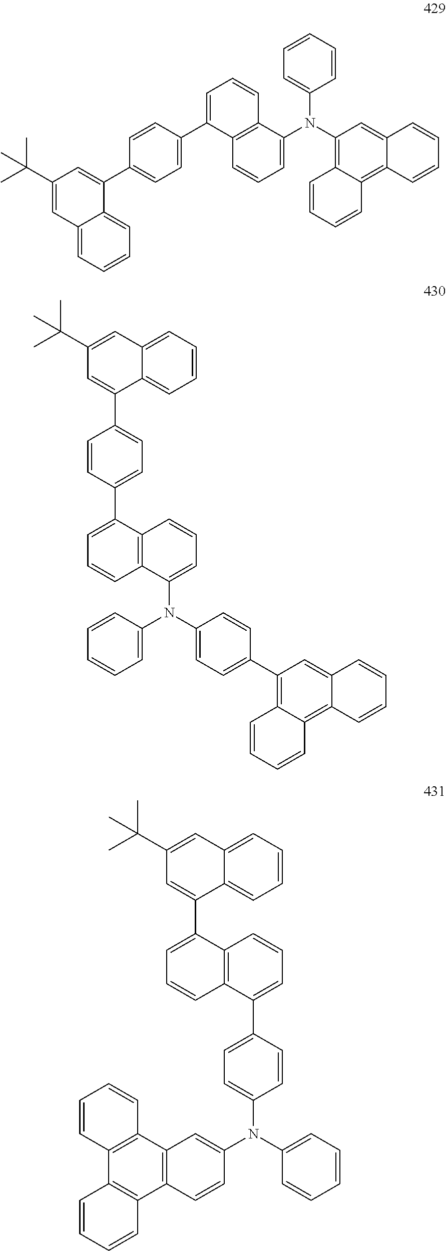 [SMILES: CC(C)(C)c1cc(-c2ccc(-c3cccc4c(N(c5ccccc5)c5cc6ccccc6c6ccccc56)cccc34)cc2)c2ccccc2c1 ; CC(C)(C)c1cc(-c2ccc(-c3cccc4c(N(c5ccccc5)c5ccc(-c6cc7ccccc7c7ccccc67)cc5)cccc34)cc2)c2ccccc2c1 ; CC(C)(C)c1cc(-c2cccc3c(-c4ccc(N(c5ccccc5)c5ccc6c7ccccc7c7ccccc7c6c5)cc4)cccc23)c2ccccc2c1]

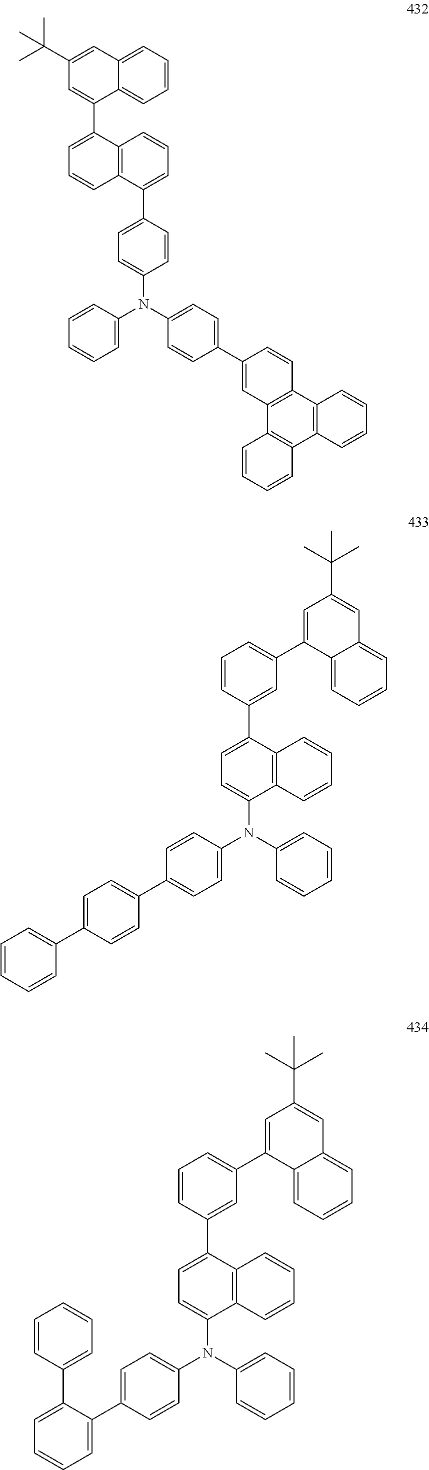 [SMILES: CC(C)(C)c1cc(-c2cccc3c(-c4ccc(N(c5ccccc5)c5ccc(-c6ccc7c8ccccc8c8ccccc8c7c6)cc5)cc4)cccc23)c2ccccc2c1 ; CC(C)(C)c1cc(-c2cccc(-c3ccc(N(c4ccccc4)c4ccc(-c5ccc(-c6ccccc6)cc5)cc4)c4ccccc34)c2)c2ccccc2c1 ; CC(C)(C)c1cc(-c2cccc(-c3ccc(N(c4ccccc4)c4ccc(-c5ccccc5-c5ccccc5)cc4)c4ccccc34)c2)c2ccccc2c1]

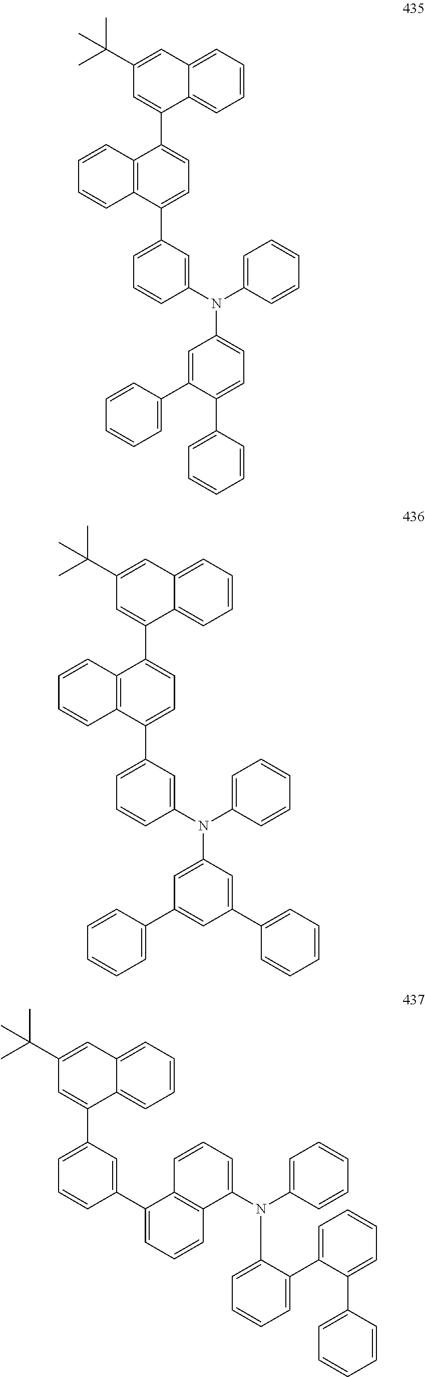 [SMILES: CC(C)(C)c1cc(-c2ccc(-c3cccc(N(c4ccccc4)c4ccc(-c5ccccc5)c(-c5ccccc5)c4)c3)c3ccccc23)c2ccccc2c1 ; CC(C)(C)c1cc(-c2ccc(-c3cccc(N(c4ccccc4)c4cc(-c5ccccc5)cc(-c5ccccc5)c4)c3)c3ccccc23)c2ccccc2c1 ; CC(C)(C)c1cc(-c2cccc(-c3cccc4c(N(c5ccccc5)c5ccccc5-c5ccccc5-c5ccccc5)cccc34)c2)c2ccccc2c1]

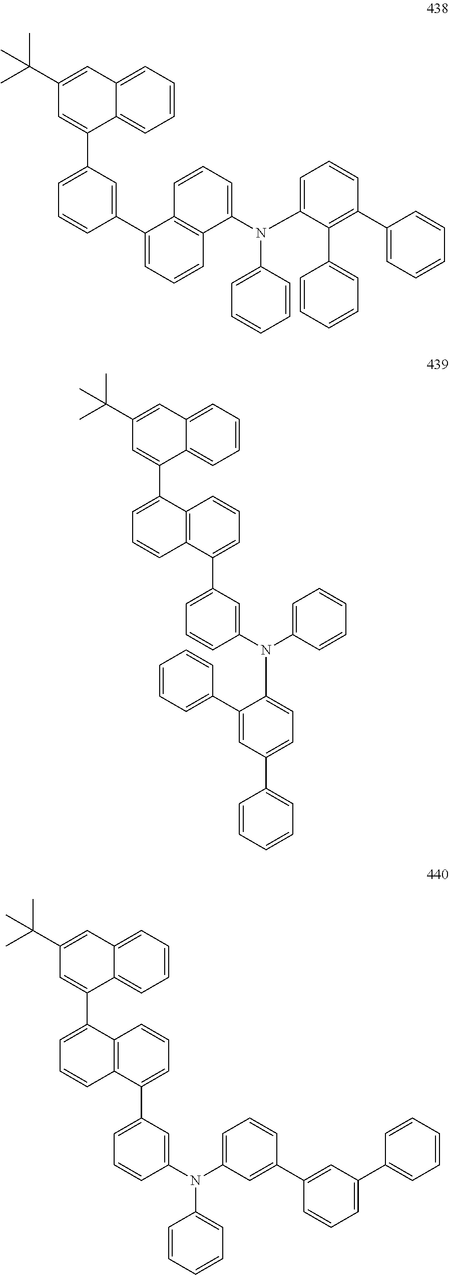 [SMILES: CC(C)(C)c1cc(-c2cccc(-c3cccc4c(N(c5ccccc5)c5cccc(-c6ccccc6)c5-c5ccccc5)cccc34)c2)c2ccccc2c1 ; CC(C)(C)c1cc(-c2cccc3c(-c4cccc(N(c5ccccc5)c5ccc(-c6ccccc6)cc5-c5ccccc5)c4)cccc23)c2ccccc2c1 ; CC(C)(C)c1cc(-c2cccc3c(-c4cccc(N(c5ccccc5)c5cccc(-c6cccc(-c7ccccc7)c6)c5)c4)cccc23)c2ccccc2c1]

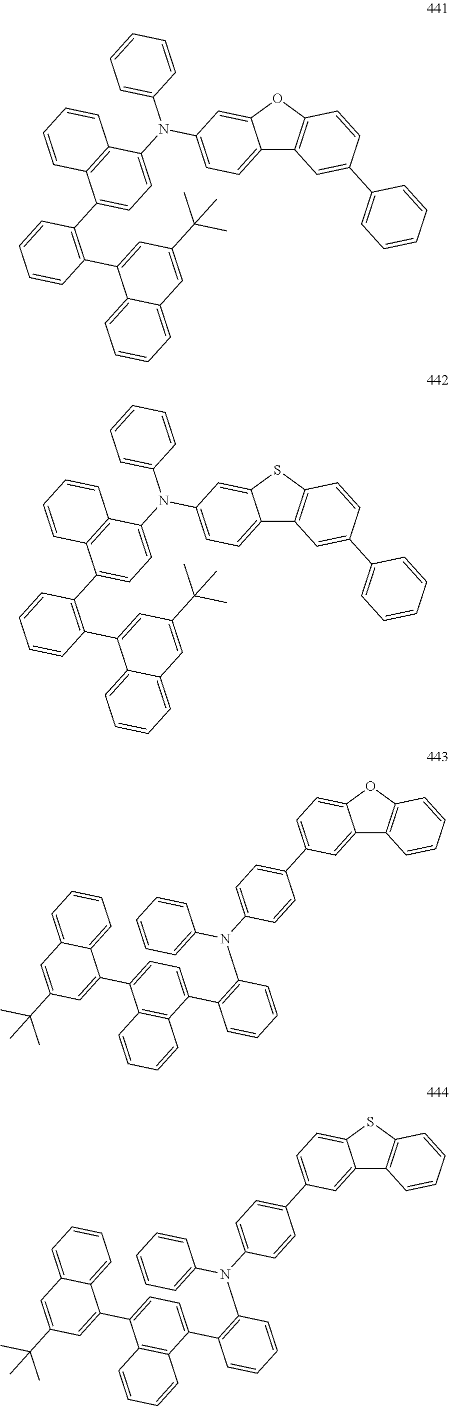

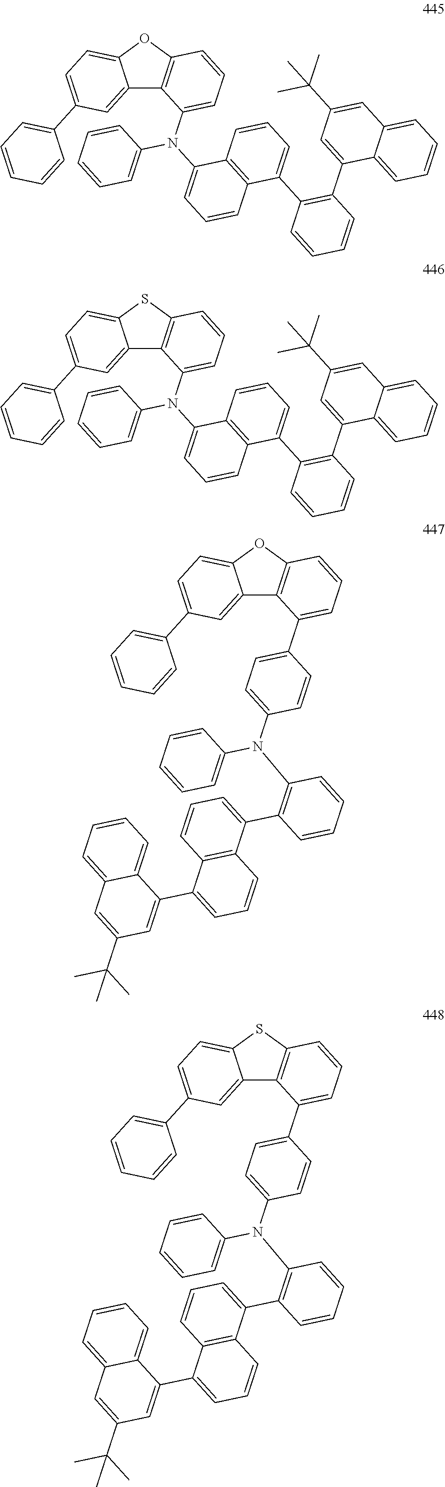

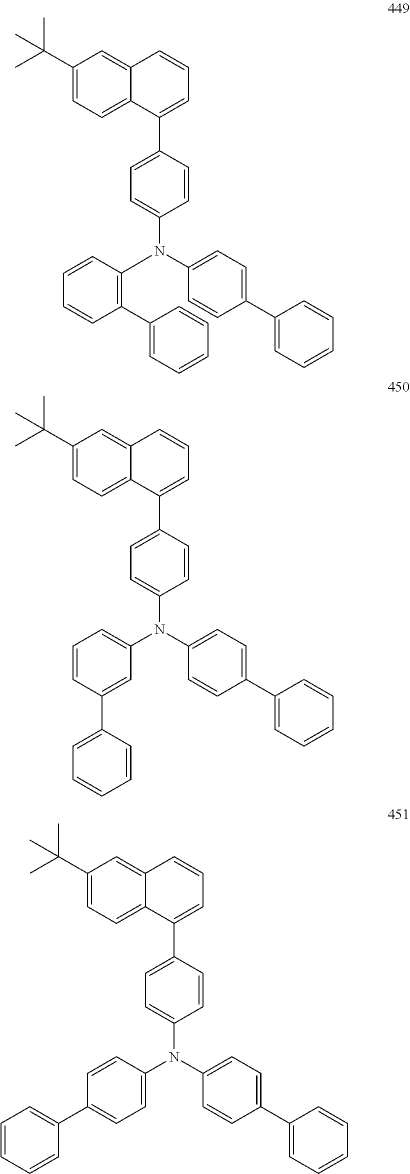

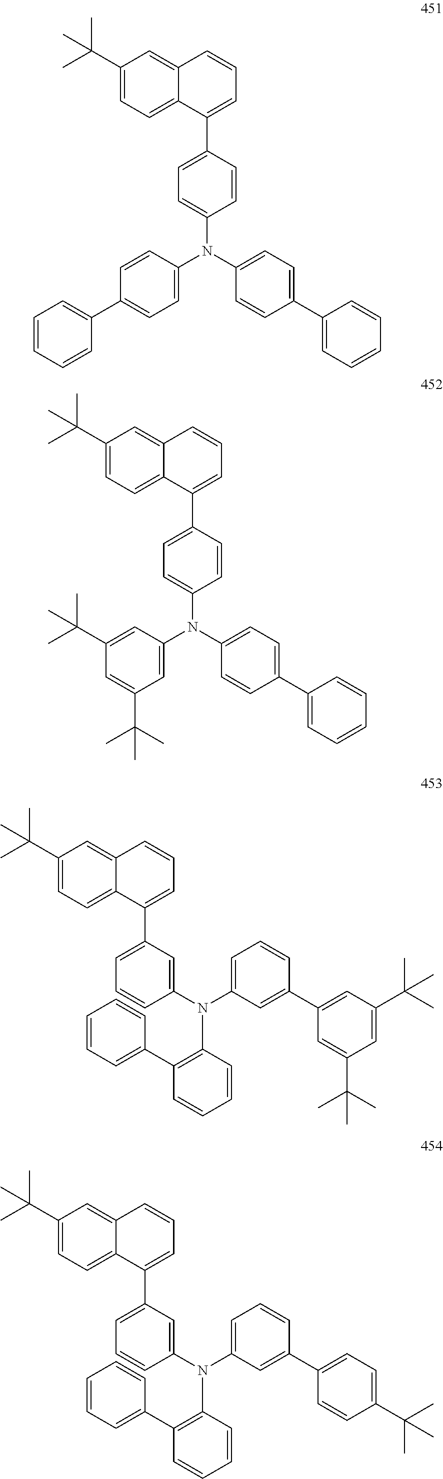

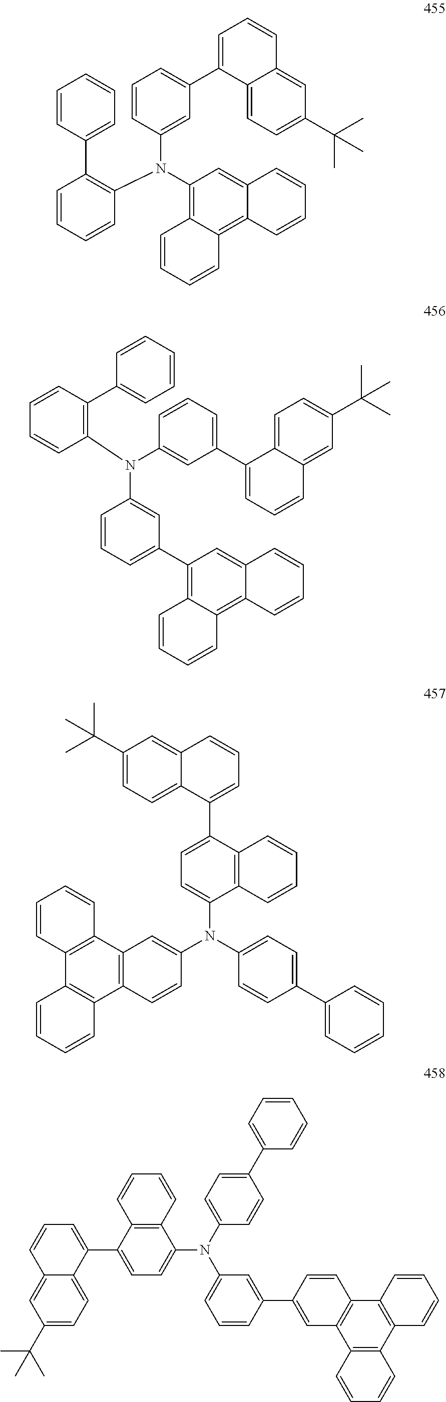

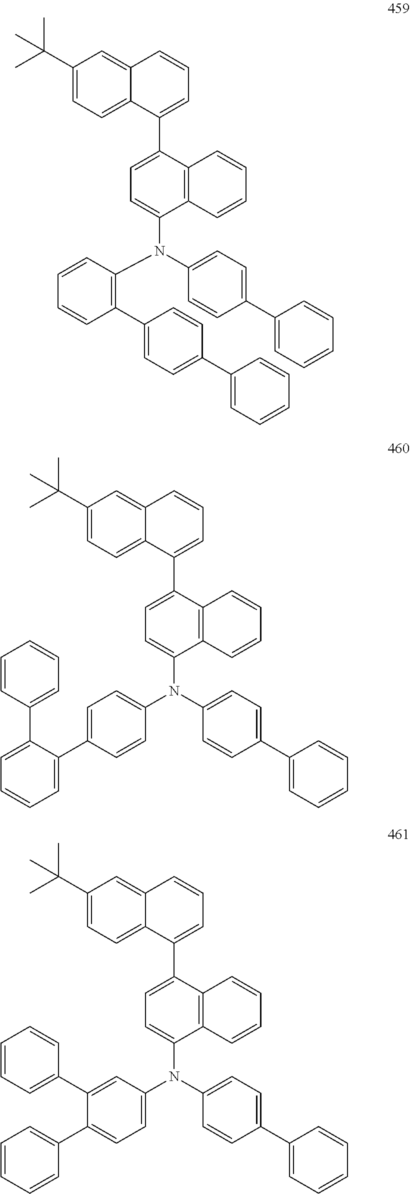

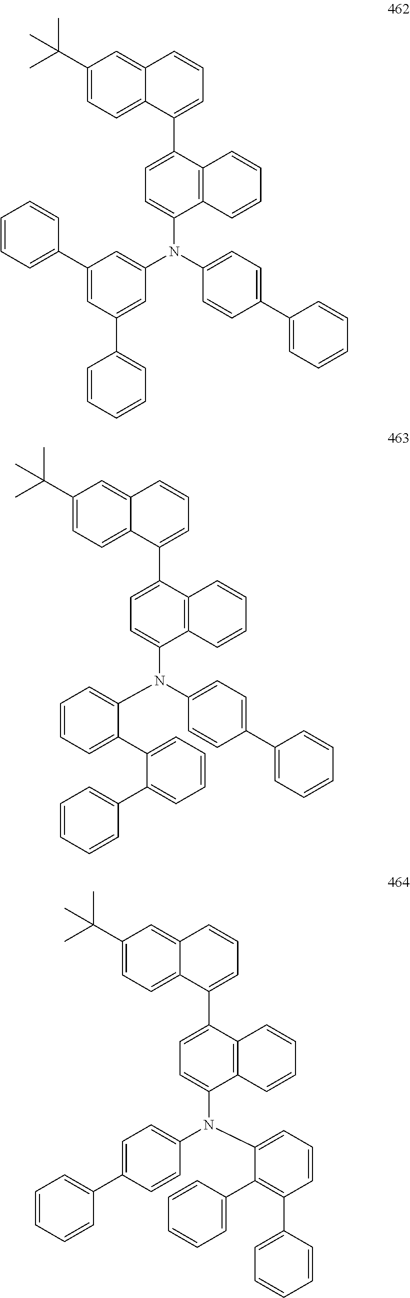

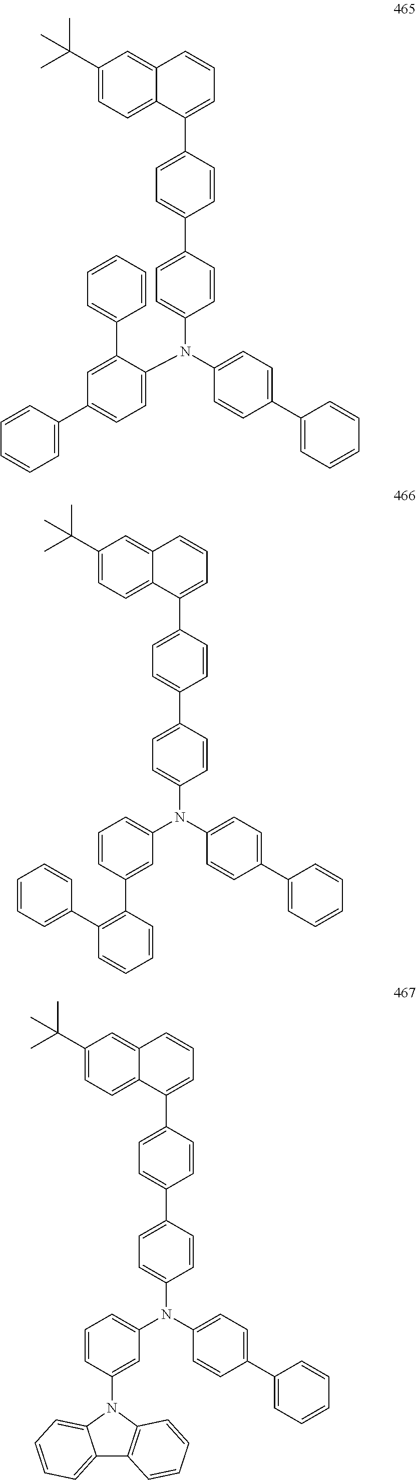 [SMILES: CC(C)(C)c1ccc2c(-c3ccc(-c4ccc(N(c5ccc(-c6ccccc6)cc5)c5ccc(-c6ccccc6)cc5-c5ccccc5)cc4)cc3)cccc2c1 ; CC(C)(C)c1ccc2c(-c3ccc(-c4ccc(N(c5ccc(-c6ccccc6)cc5)c5cccc(-c6ccccc6-c6ccccc6)c5)cc4)cc3)cccc2c1 ; CC(C)(C)c1ccc2c(-c3ccc(-c4ccc(N(c5ccc(-c6ccccc6)cc5)c5cccc(-n6c7ccccc7c7ccccc76)c5)cc4)cc3)cccc2c1]

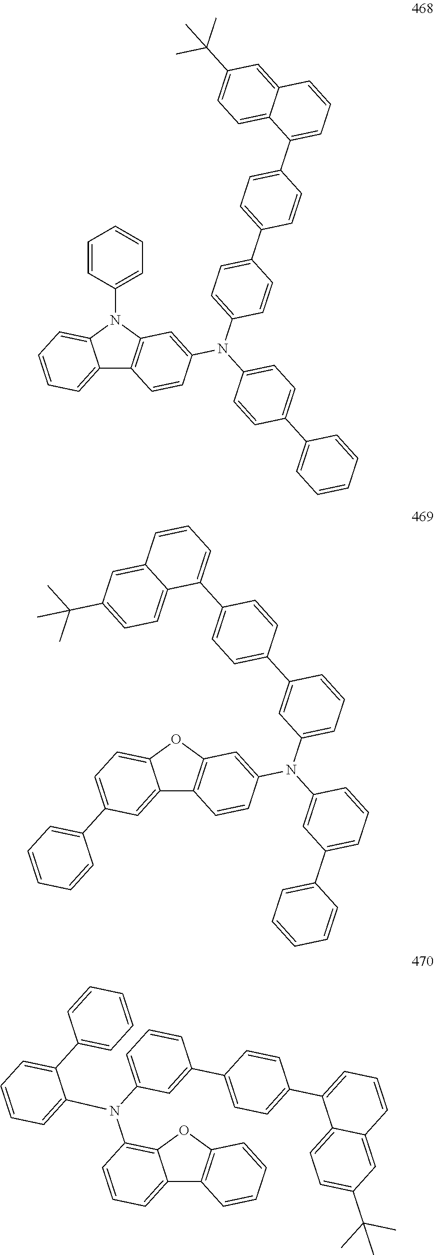 [SMILES: CC(C)(C)c1ccc2c(-c3ccc(-c4ccc(N(c5ccc(-c6ccccc6)cc5)c5ccc6c7ccccc7n(-c7ccccc7)c6c5)cc4)cc3)cccc2c1 ; CC(C)(C)c1ccc2c(-c3ccc(-c4cccc(N(c5cccc(-c6ccccc6)c5)c5ccc6c(c5)oc5ccc(-c7ccccc7)cc56)c4)cc3)cccc2c1 ; CC(C)(C)c1ccc2c(-c3ccc(-c4cccc(N(c5ccccc5-c5ccccc5)c5cccc6c5oc5ccccc56)c4)cc3)cccc2c1]

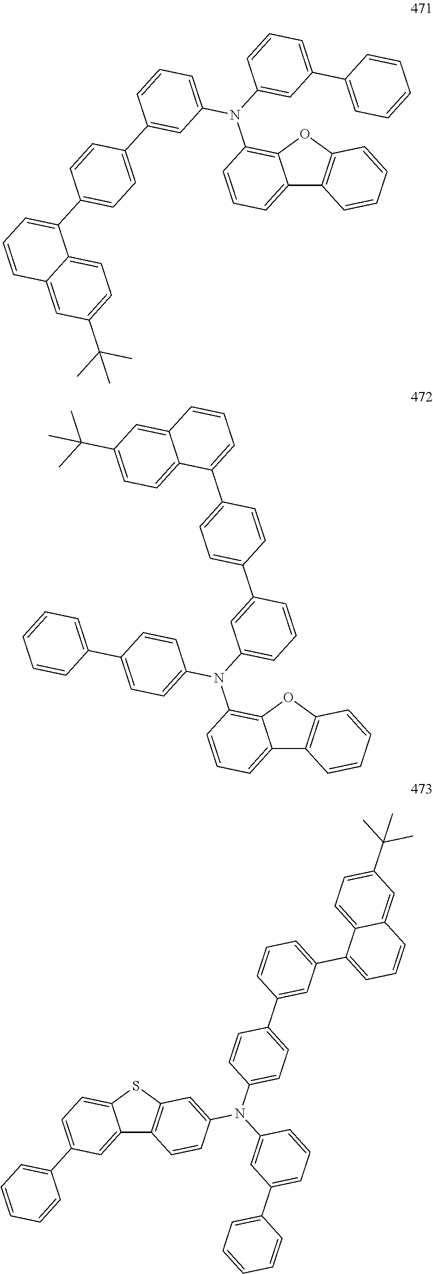

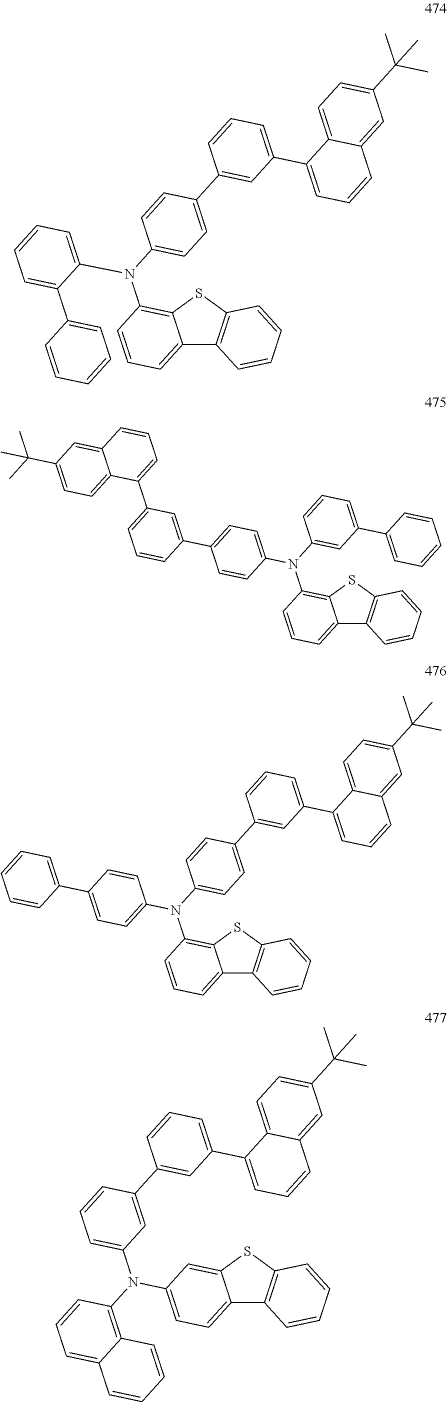

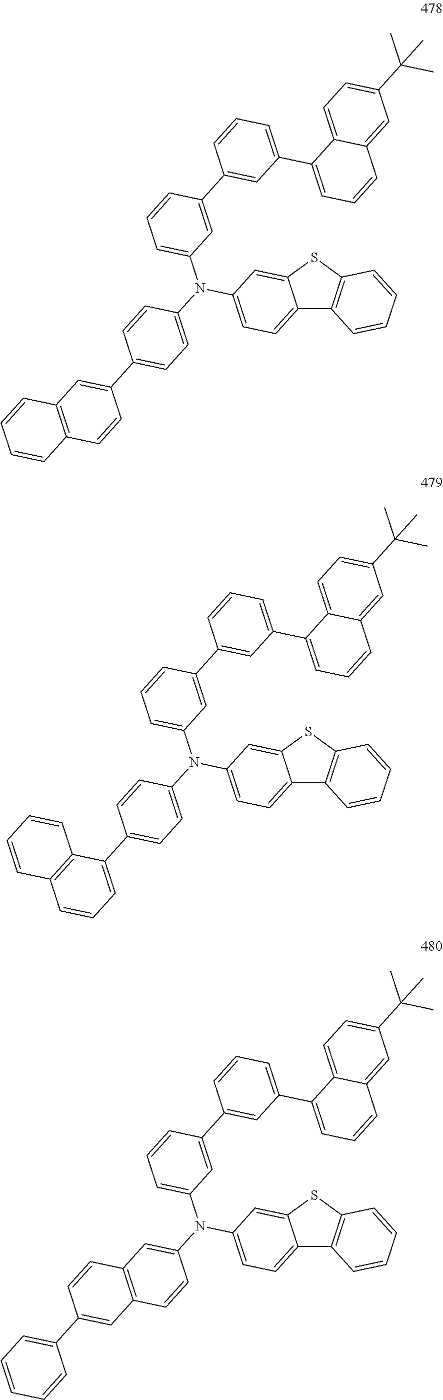 [SMILES: CC(C)(C)c1ccc2c(-c3cccc(-c4cccc(N(c5ccc(-c6ccc7ccccc7c6)cc5)c5ccc6c(c5)sc5ccccc56)c4)c3)cccc2c1 ; CC(C)(C)c1ccc2c(-c3cccc(-c4cccc(N(c5ccc(-c6cccc7ccccc67)cc5)c5ccc6c(c5)sc5ccccc56)c4)c3)cccc2c1 ; CC(C)(C)c1ccc2c(-c3cccc(-c4cccc(N(c5ccc6cc(-c7ccccc7)ccc6c5)c5ccc6c(c5)sc5ccccc56)c4)c3)cccc2c1]

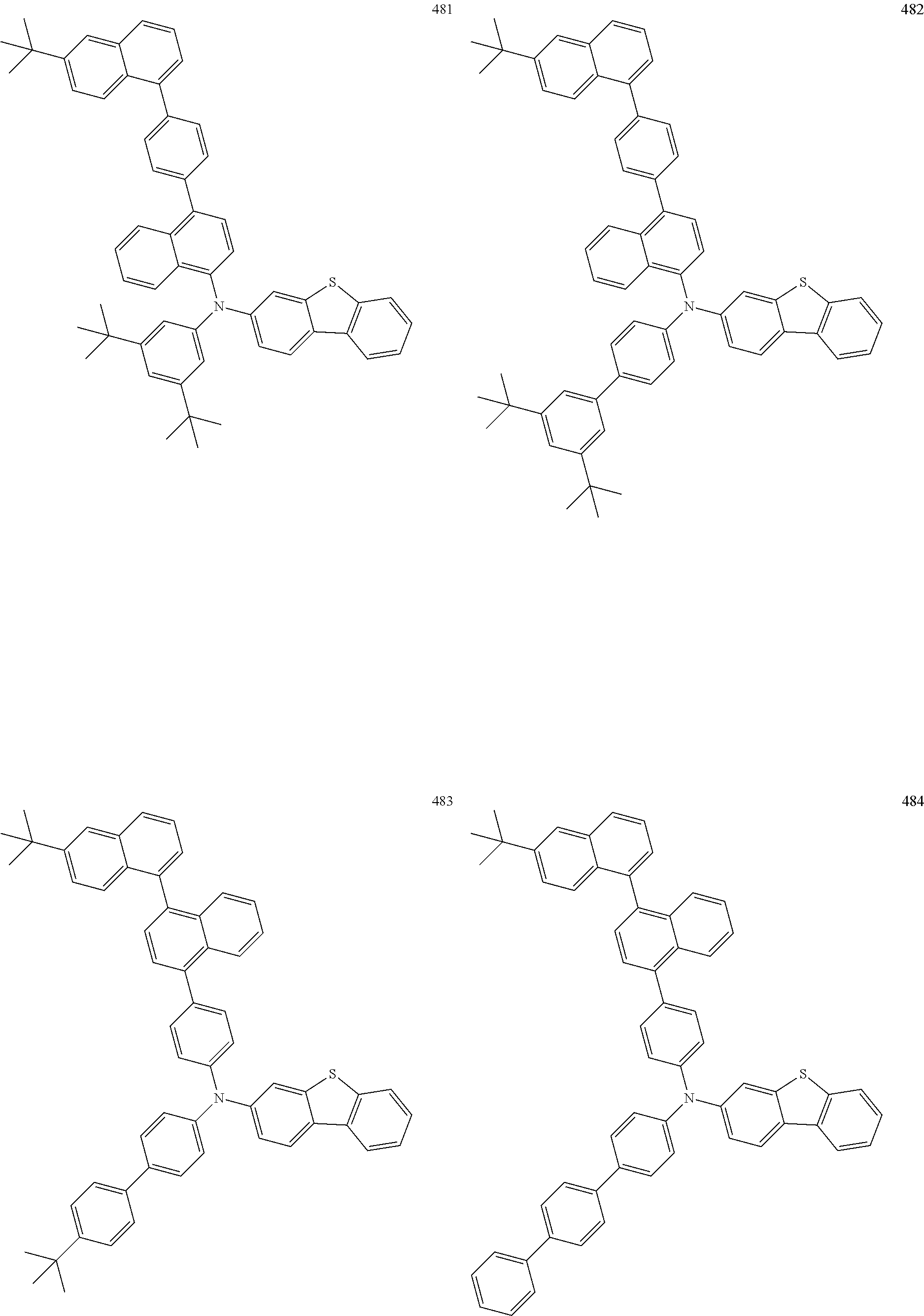 [SMILES: CC(C)(C)c1cc(N(c2ccc3c(c2)sc2ccccc23)c2ccc(-c3ccc(-c4cccc5cc(C(C)(C)C)ccc45)cc3)c3ccccc23)cc(C(C)(C)C)c1 ; CC(C)(C)c1cc(-c2ccc(N(c3ccc4c(c3)sc3ccccc34)c3ccc(-c4ccc(-c5cccc6cc(C(C)(C)C)ccc56)cc4)c4ccccc34)cc2)cc(C(C)(C)C)c1 ; CC(C)(C)c1ccc(-c2ccc(N(c3ccc(-c4ccc(-c5cccc6cc(C(C)(C)C)ccc56)c5ccccc45)cc3)c3ccc4c(c3)sc3ccccc34)cc2)cc1 ; CC(C)(C)c1ccc2c(-c3ccc(-c4ccc(N(c5ccc(-c6ccc(-c7ccccc7)cc6)cc5)c5ccc6c(c5)sc5ccccc56)cc4)c4ccccc34)cccc2c1]

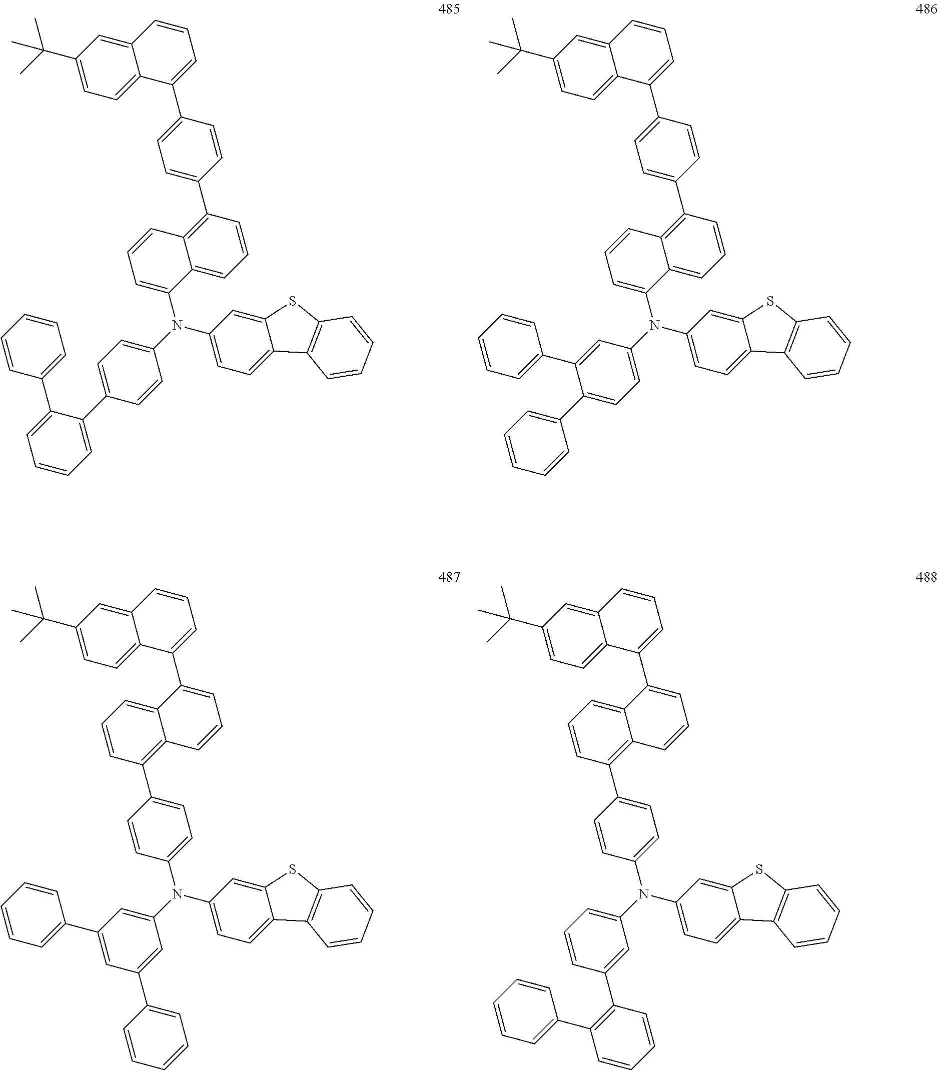 [SMILES: CC(C)(C)c1ccc2c(-c3ccc(-c4cccc5c(N(c6ccc(-c7ccccc7-c7ccccc7)cc6)c6ccc7c(c6)sc6ccccc67)cccc45)cc3)cccc2c1 ; CC(C)(C)c1ccc2c(-c3ccc(-c4cccc5c(N(c6ccc(-c7ccccc7)c(-c7ccccc7)c6)c6ccc7c(c6)sc6ccccc67)cccc45)cc3)cccc2c1 ; CC(C)(C)c1ccc2c(-c3cccc4c(-c5ccc(N(c6cc(-c7ccccc7)cc(-c7ccccc7)c6)c6ccc7c(c6)sc6ccccc67)cc5)cccc34)cccc2c1 ; CC(C)(C)c1ccc2c(-c3cccc4c(-c5ccc(N(c6cccc(-c7ccccc7-c7ccccc7)c6)c6ccc7c(c6)sc6ccccc67)cc5)cccc34)cccc2c1]

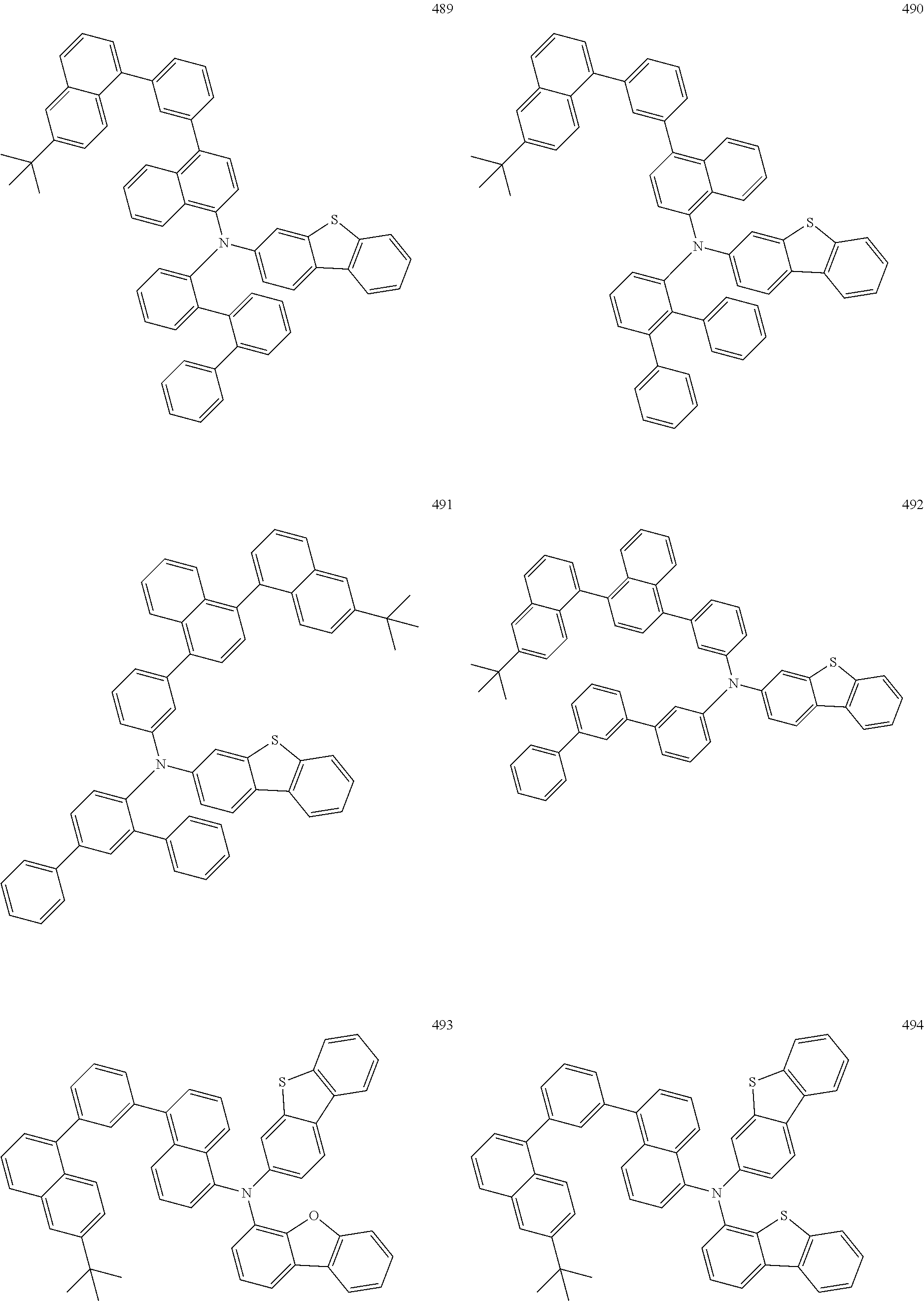

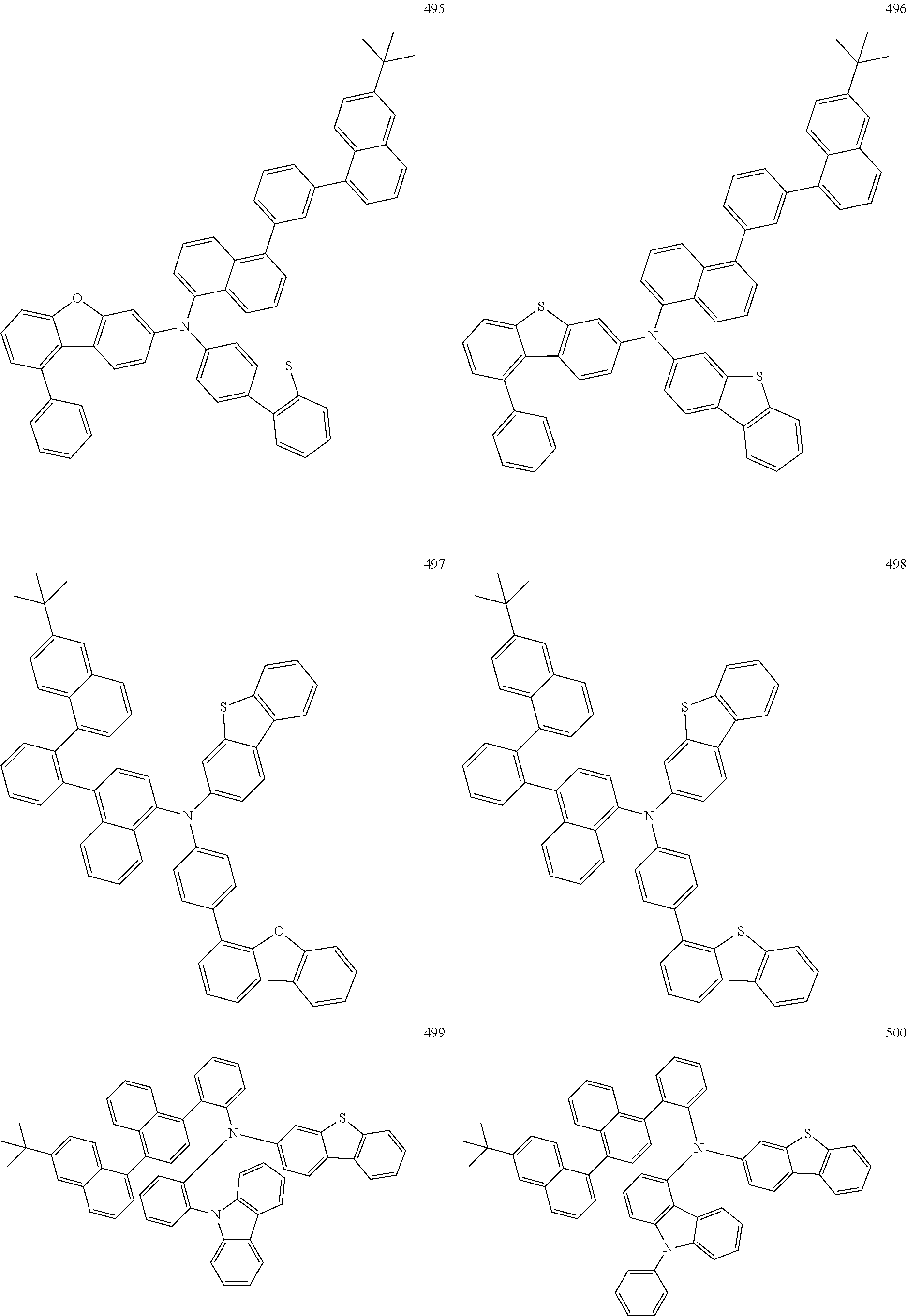

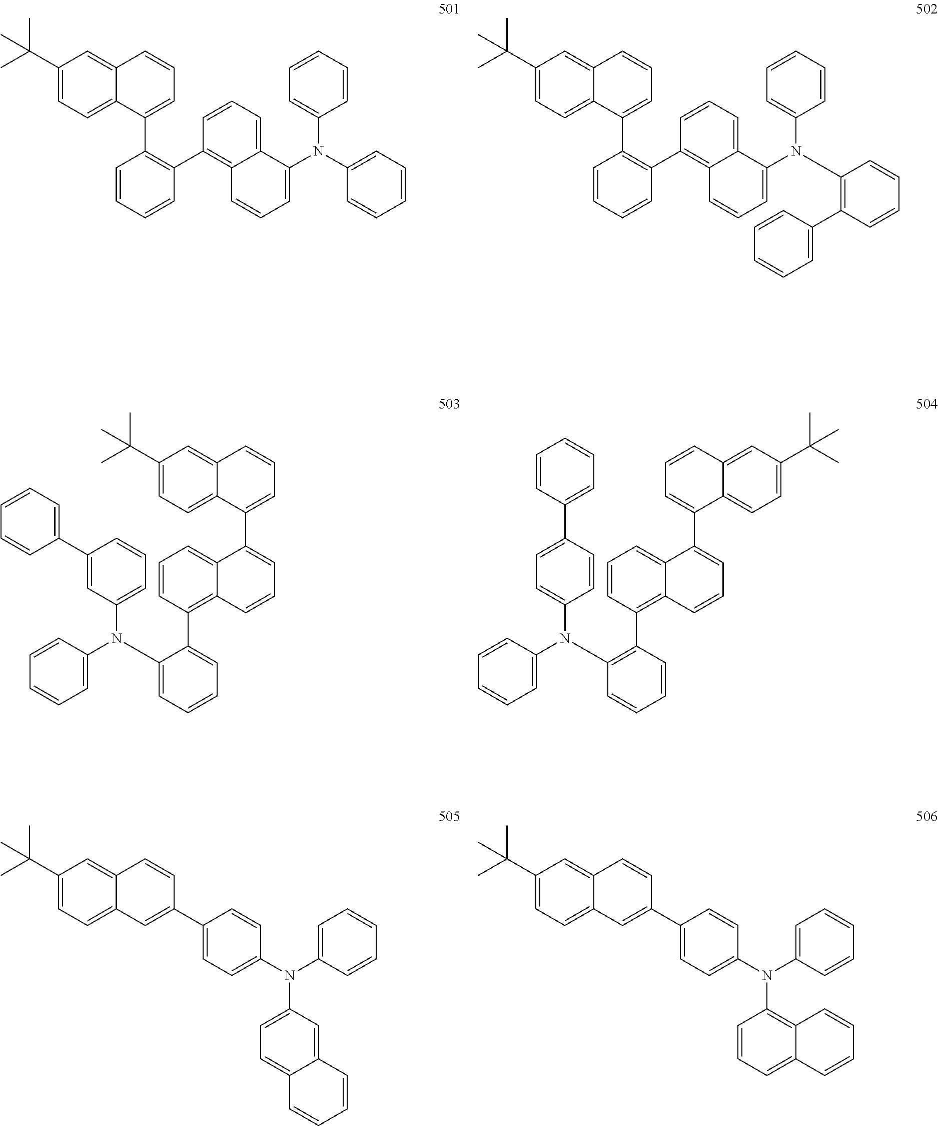 [SMILES: CC(C)(C)c1ccc2c(-c3ccccc3-c3cccc4c(N(c5ccccc5)c5ccccc5)cccc34)cccc2c1 ; CC(C)(C)c1ccc2c(-c3ccccc3-c3cccc4c(N(c5ccccc5)c5ccccc5-c5ccccc5)cccc34)cccc2c1 ; CC(C)(C)c1ccc2c(-c3cccc4c(-c5ccccc5N(c5ccccc5)c5cccc(-c6ccccc6)c5)cccc34)cccc2c1 ; CC(C)(C)c1ccc2c(-c3cccc4c(-c5ccccc5N(c5ccccc5)c5ccc(-c6ccccc6)cc5)cccc34)cccc2c1 ; CC(C)(C)c1ccc2cc(-c3ccc(N(c4ccccc4)c4ccc5ccccc5c4)cc3)ccc2c1 ; CC(C)(C)c1ccc2cc(-c3ccc(N(c4ccccc4)c4cccc5ccccc45)cc3)ccc2c1]

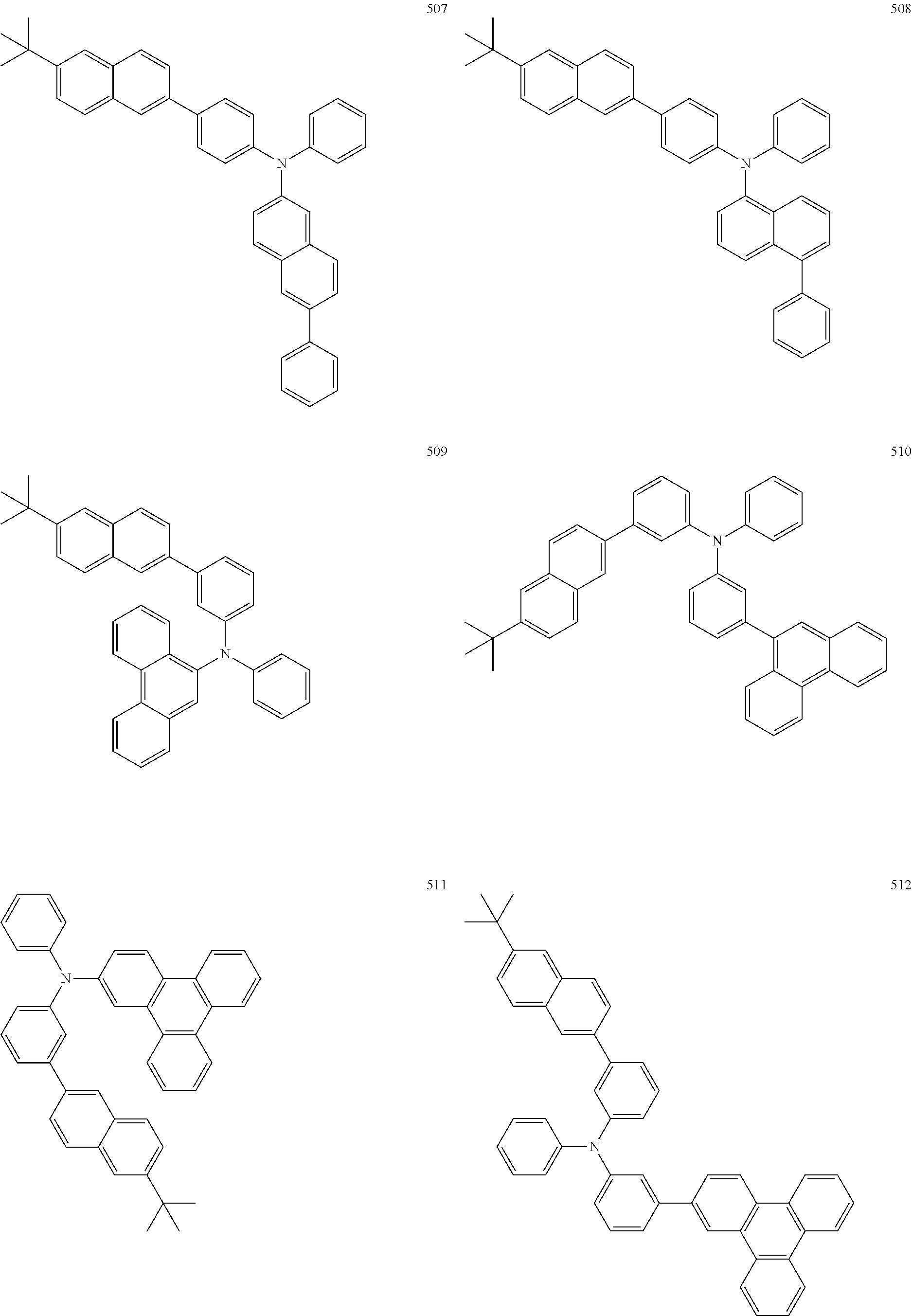 [SMILES: CC(C)(C)c1ccc2cc(-c3ccc(N(c4ccccc4)c4ccc5cc(-c6ccccc6)ccc5c4)cc3)ccc2c1 ; CC(C)(C)c1ccc2cc(-c3ccc(N(c4ccccc4)c4cccc5c(-c6ccccc6)cccc45)cc3)ccc2c1 ; CC(C)(C)c1ccc2cc(-c3cccc(N(c4ccccc4)c4cc5ccccc5c5ccccc45)c3)ccc2c1 ; CC(C)(C)c1ccc2cc(-c3cccc(N(c4ccccc4)c4cccc(-c5cc6ccccc6c6ccccc56)c4)c3)ccc2c1 ; CC(C)(C)c1ccc2cc(-c3cccc(N(c4ccccc4)c4ccc5c6ccccc6c6ccccc6c5c4)c3)ccc2c1 ; CC(C)(C)c1ccc2cc(-c3cccc(N(c4ccccc4)c4cccc(-c5ccc6c7ccccc7c7ccccc7c6c5)c4)c3)ccc2c1]

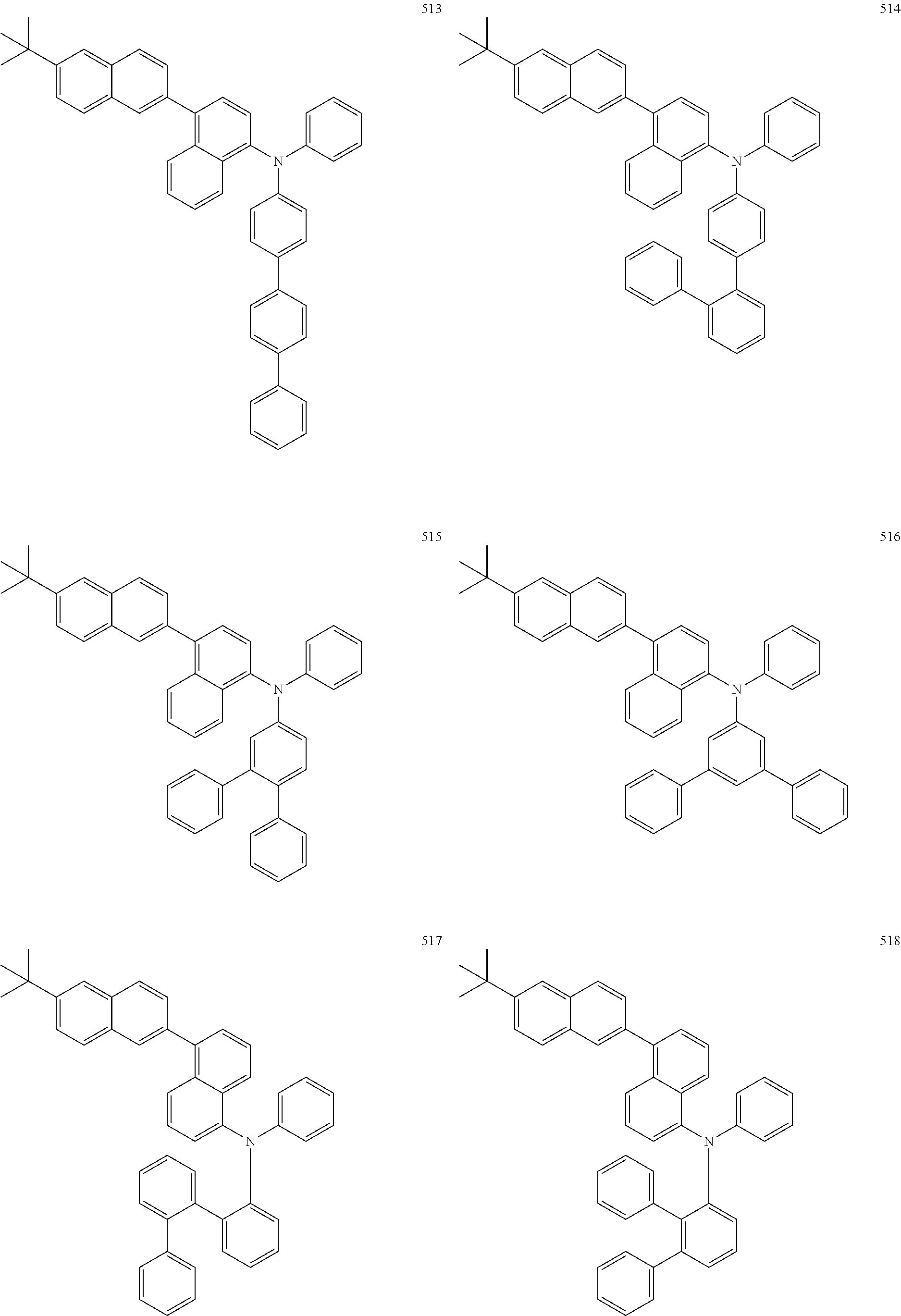 [SMILES: CC(C)(C)c1ccc2cc(-c3ccc(N(c4ccccc4)c4ccc(-c5ccc(-c6ccccc6)cc5)cc4)c4ccccc34)ccc2c1 ; CC(C)(C)c1ccc2cc(-c3ccc(N(c4ccccc4)c4ccc(-c5ccccc5-c5ccccc5)cc4)c4ccccc34)ccc2c1 ; CC(C)(C)c1ccc2cc(-c3ccc(N(c4ccccc4)c4ccc(-c5ccccc5)c(-c5ccccc5)c4)c4ccccc34)ccc2c1 ; CC(C)(C)c1ccc2cc(-c3ccc(N(c4ccccc4)c4cc(-c5ccccc5)cc(-c5ccccc5)c4)c4ccccc34)ccc2c1 ; CC(C)(C)c1ccc2cc(-c3cccc4c(N(c5ccccc5)c5ccccc5-c5ccccc5-c5ccccc5)cccc34)ccc2c1 ; CC(C)(C)c1ccc2cc(-c3cccc4c(N(c5ccccc5)c5cccc(-c6ccccc6)c5-c5ccccc5)cccc34)ccc2c1]

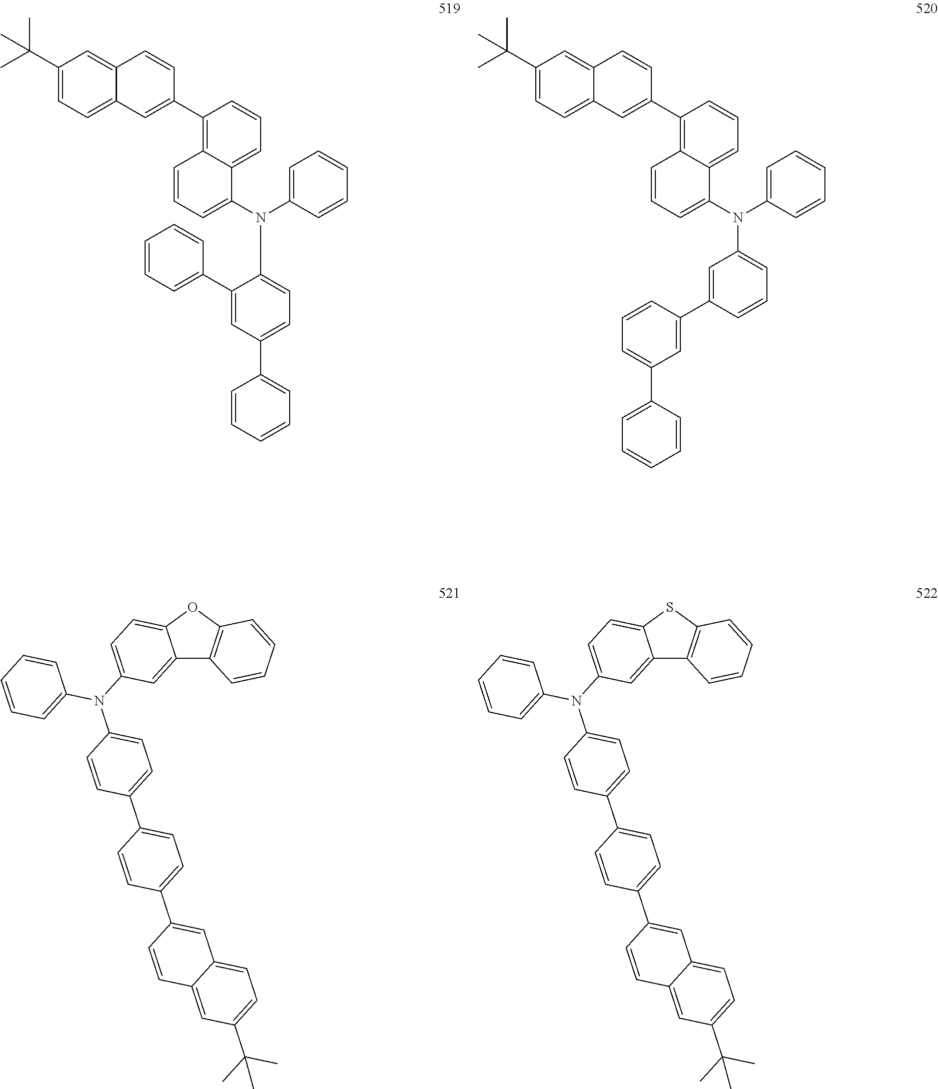 [SMILES: CC(C)(C)c1ccc2cc(-c3cccc4c(N(c5ccccc5)c5ccc(-c6ccccc6)cc5-c5ccccc5)cccc34)ccc2c1 ; CC(C)(C)c1ccc2cc(-c3cccc4c(N(c5ccccc5)c5cccc(-c6cccc(-c7ccccc7)c6)c5)cccc34)ccc2c1 ; CC(C)(C)c1ccc2cc(-c3ccc(-c4ccc(N(c5ccccc5)c5ccc6oc7ccccc7c6c5)cc4)cc3)ccc2c1 ; CC(C)(C)c1ccc2cc(-c3ccc(-c4ccc(N(c5ccccc5)c5ccc6sc7ccccc7c6c5)cc4)cc3)ccc2c1]

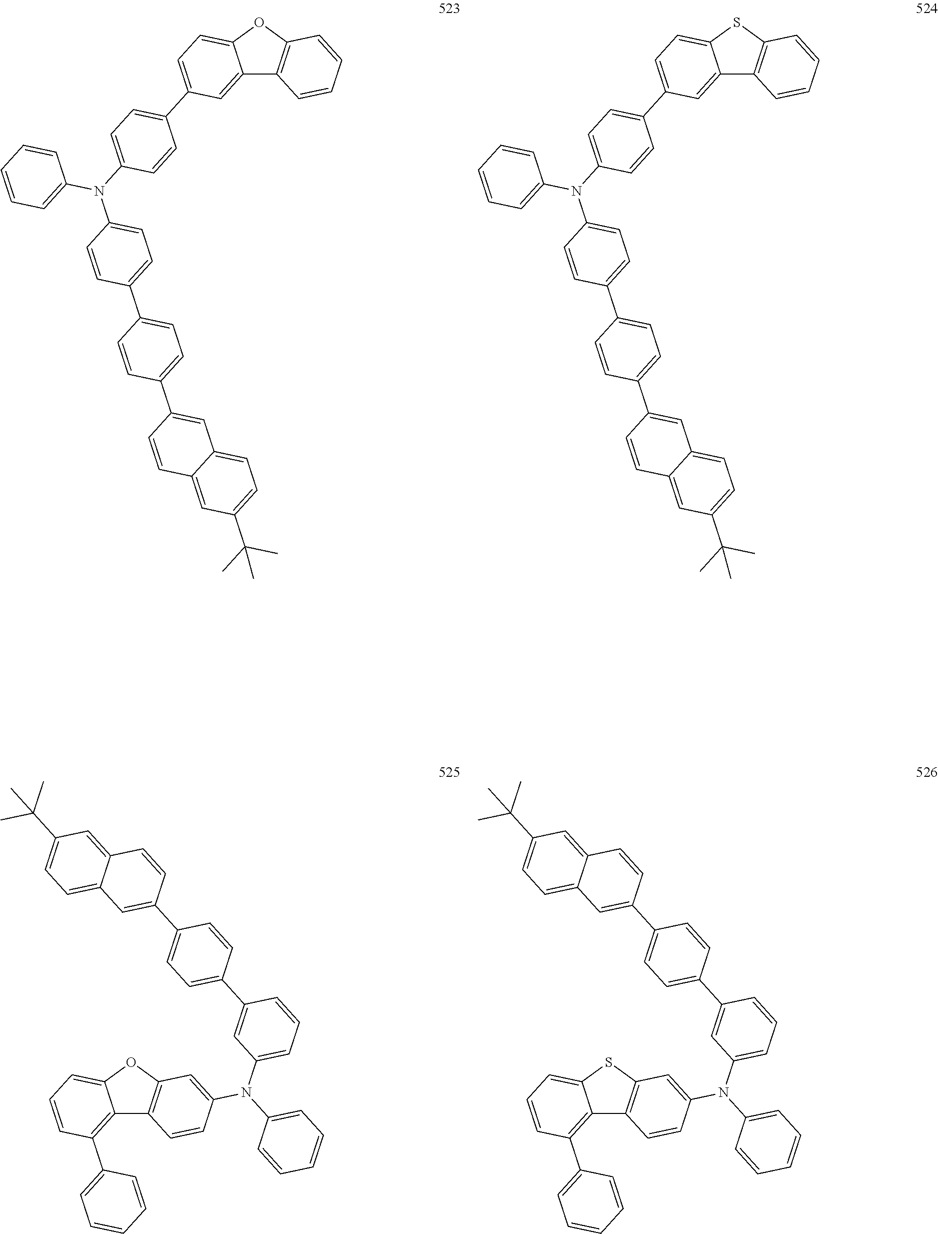 [SMILES: CC(C)(C)c1ccc2cc(-c3ccc(-c4ccc(N(c5ccccc5)c5ccc(-c6ccc7oc8ccccc8c7c6)cc5)cc4)cc3)ccc2c1 ; CC(C)(C)c1ccc2cc(-c3ccc(-c4ccc(N(c5ccccc5)c5ccc(-c6ccc7sc8ccccc8c7c6)cc5)cc4)cc3)ccc2c1 ; CC(C)(C)c1ccc2cc(-c3ccc(-c4cccc(N(c5ccccc5)c5ccc6c(c5)oc5cccc(-c7ccccc7)c56)c4)cc3)ccc2c1 ; CC(C)(C)c1ccc2cc(-c3ccc(-c4cccc(N(c5ccccc5)c5ccc6c(c5)sc5cccc(-c7ccccc7)c56)c4)cc3)ccc2c1]

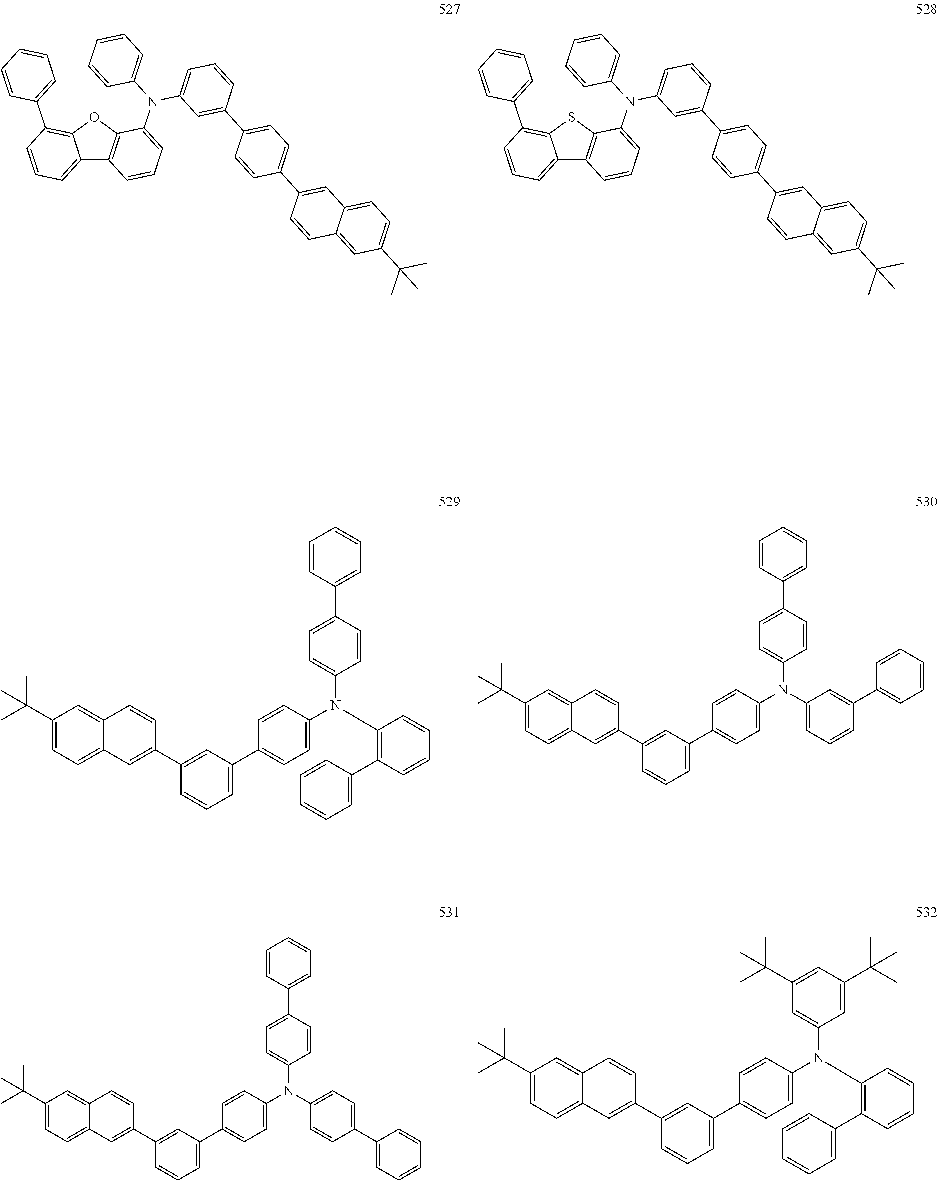

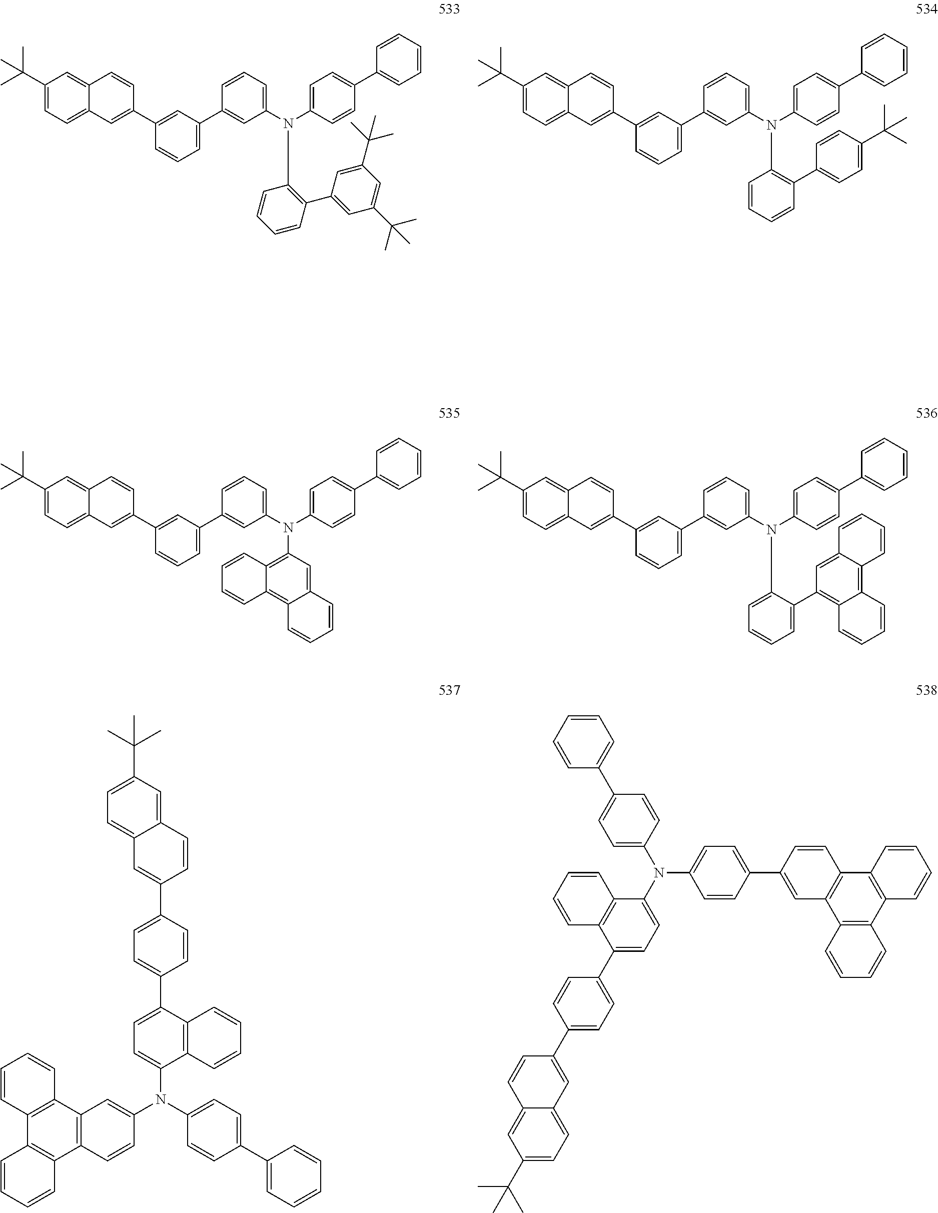

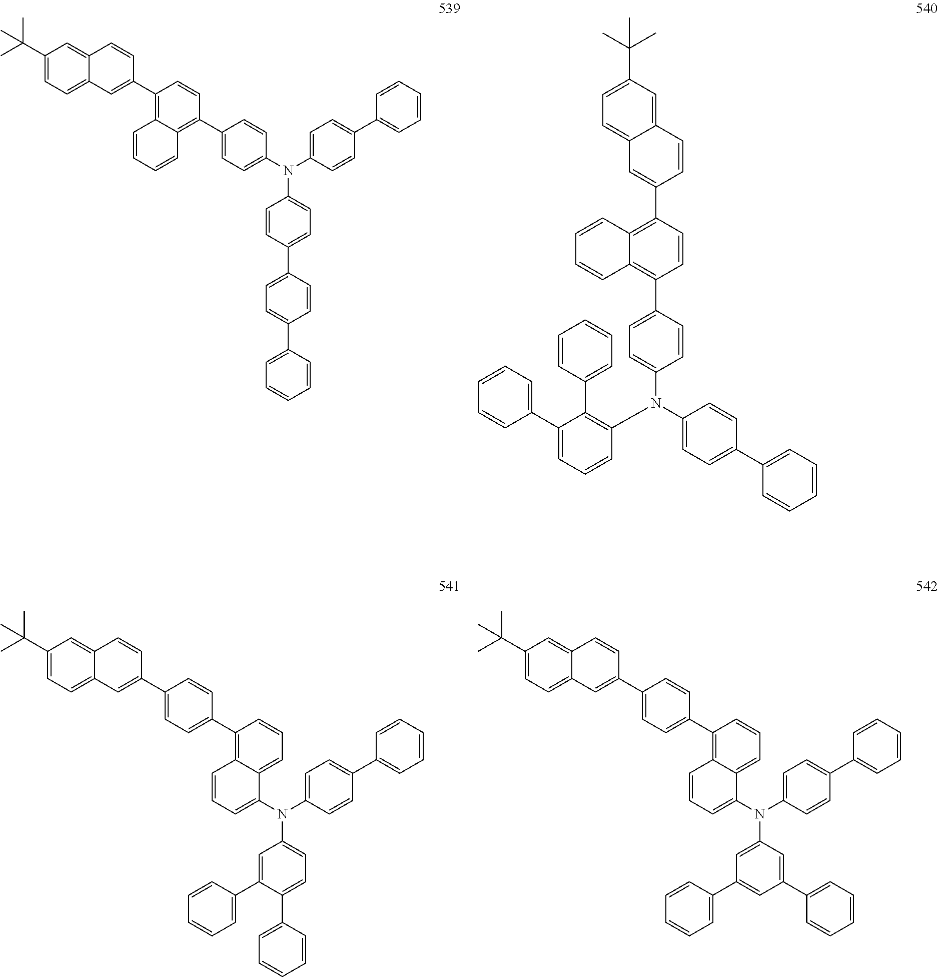 [SMILES: CC(C)(C)c1ccc2cc(-c3ccc(-c4ccc(N(c5ccc(-c6ccccc6)cc5)c5ccc(-c6ccc(-c7ccccc7)cc6)cc5)cc4)c4ccccc34)ccc2c1 ; CC(C)(C)c1ccc2cc(-c3ccc(-c4ccc(N(c5ccc(-c6ccccc6)cc5)c5cccc(-c6ccccc6)c5-c5ccccc5)cc4)c4ccccc34)ccc2c1 ; CC(C)(C)c1ccc2cc(-c3ccc(-c4cccc5c(N(c6ccc(-c7ccccc7)cc6)c6ccc(-c7ccccc7)c(-c7ccccc7)c6)cccc45)cc3)ccc2c1 ; CC(C)(C)c1ccc2cc(-c3ccc(-c4cccc5c(N(c6ccc(-c7ccccc7)cc6)c6cc(-c7ccccc7)cc(-c7ccccc7)c6)cccc45)cc3)ccc2c1]

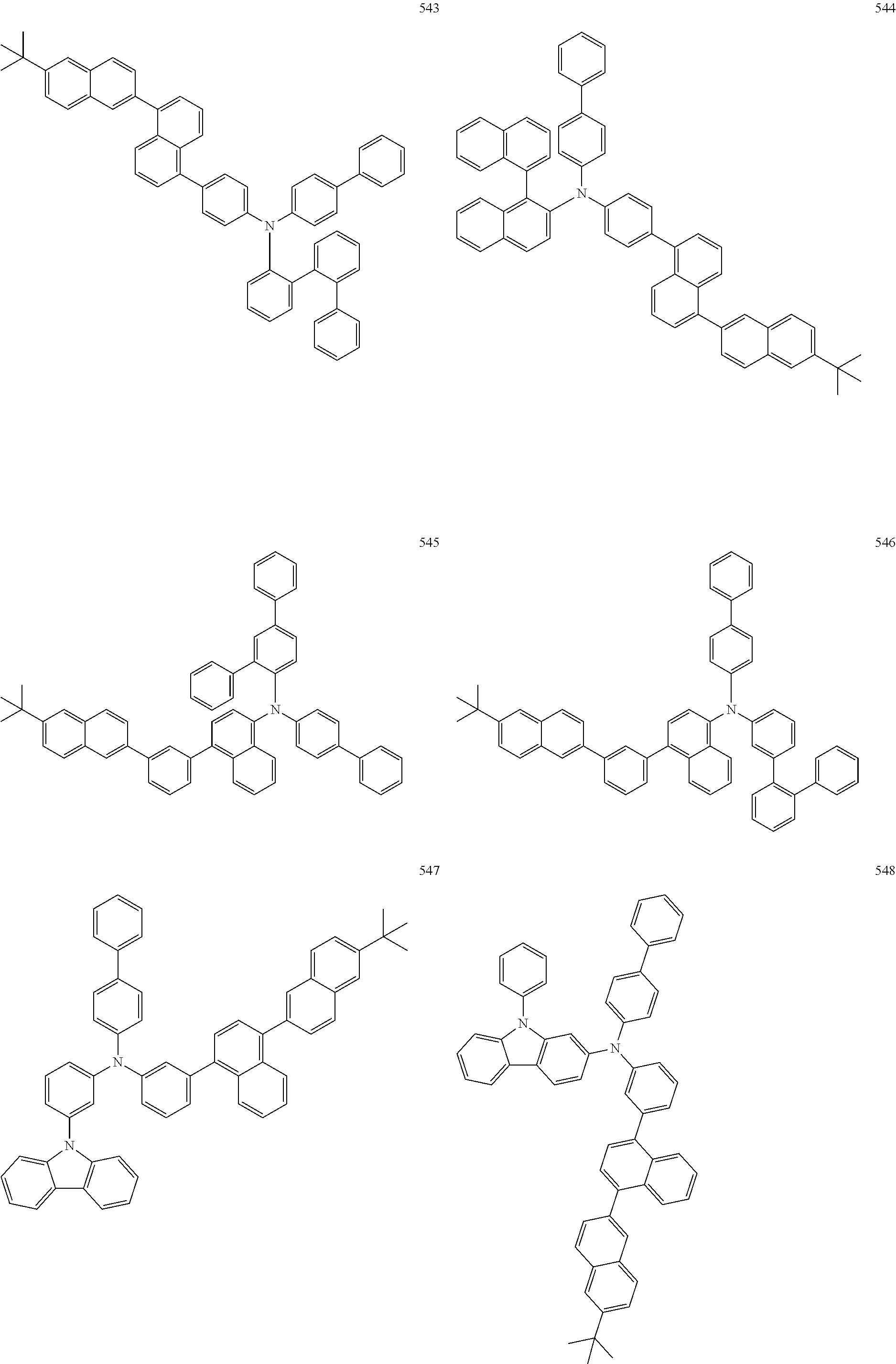

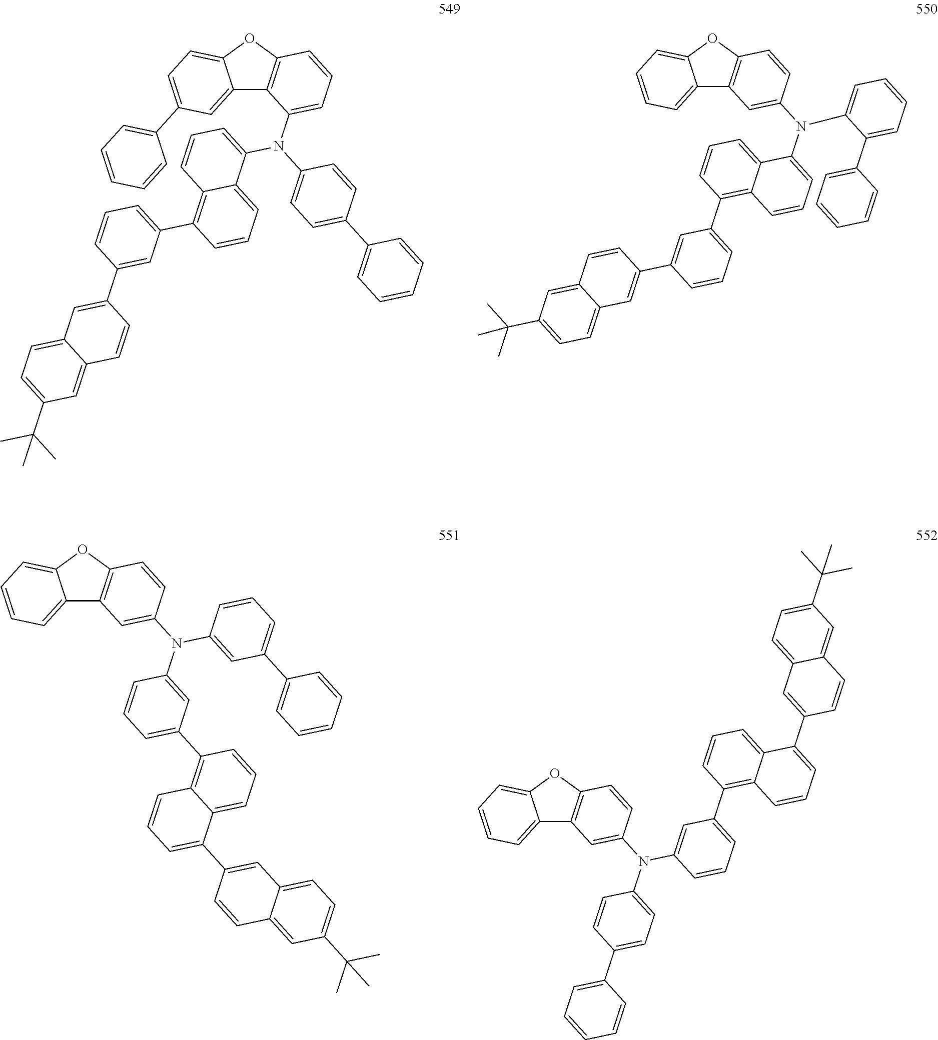 [SMILES: CC(C)(C)c1ccc2cc(-c3cccc(-c4cccc5c(N(c6ccc(-c7ccccc7)cc6)c6cccc7oc8ccc(-c9ccccc9)cc8c67)cccc45)c3)ccc2c1 ; CC(C)(C)c1ccc2cc(-c3cccc(-c4cccc5c(N(c6ccc7oc8ccccc8c7c6)c6ccccc6-c6ccccc6)cccc45)c3)ccc2c1 ; CC(C)(C)c1ccc2cc(-c3cccc4c(-c5cccc(N(c6cccc(-c7ccccc7)c6)c6ccc7oc8ccccc8c7c6)c5)cccc34)ccc2c1 ; CC(C)(C)c1ccc2cc(-c3cccc4c(-c5cccc(N(c6ccc(-c7ccccc7)cc6)c6ccc7oc8ccccc8c7c6)c5)cccc34)ccc2c1]

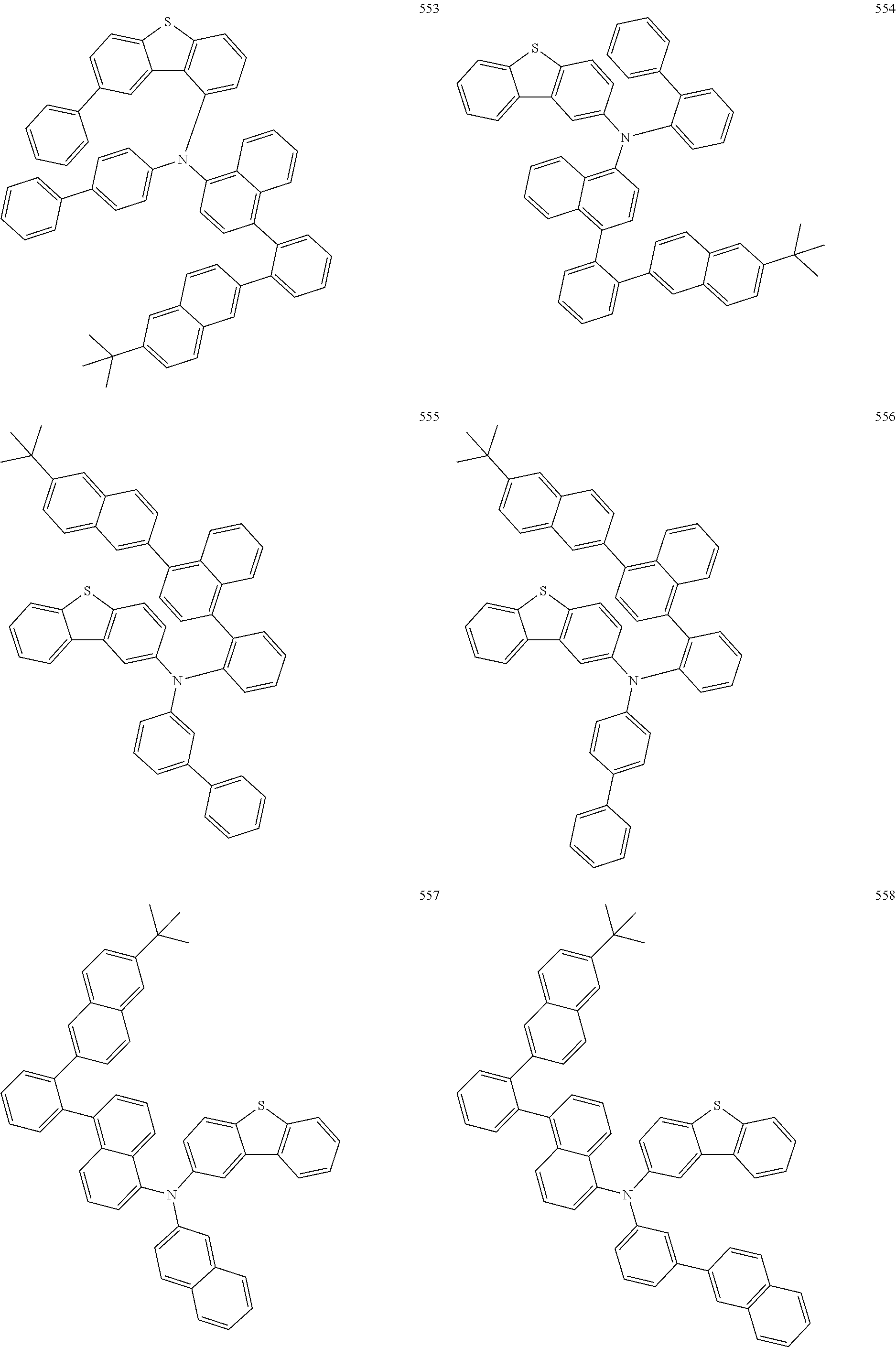

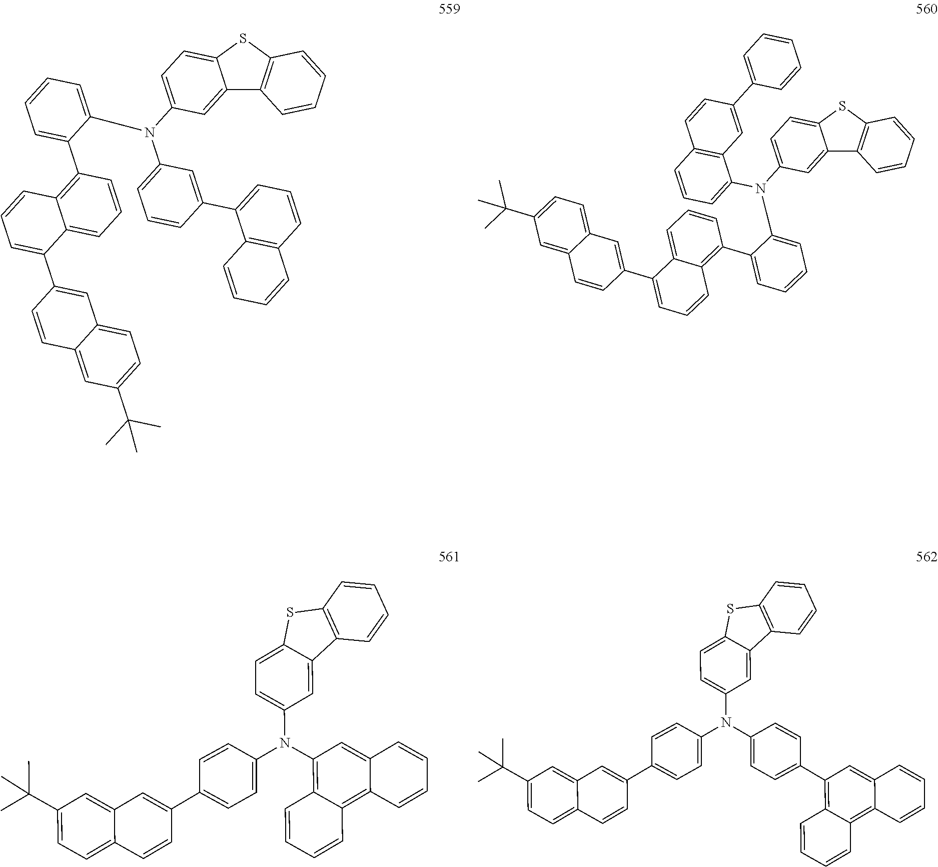 [SMILES: CC(C)(C)c1ccc2cc(-c3cccc4c(-c5ccccc5N(c5cccc(-c6cccc7ccccc67)c5)c5ccc6sc7ccccc7c6c5)cccc34)ccc2c1 ; CC(C)(C)c1ccc2cc(-c3cccc4c(-c5ccccc5N(c5ccc6sc7ccccc7c6c5)c5cccc6ccc(-c7ccccc7)cc56)cccc34)ccc2c1 ; CC(C)(C)c1ccc2ccc(-c3ccc(N(c4ccc5sc6ccccc6c5c4)c4cc5ccccc5c5ccccc45)cc3)cc2c1 ; CC(C)(C)c1ccc2ccc(-c3ccc(N(c4ccc(-c5cc6ccccc6c6ccccc56)cc4)c4ccc5sc6ccccc6c5c4)cc3)cc2c1]

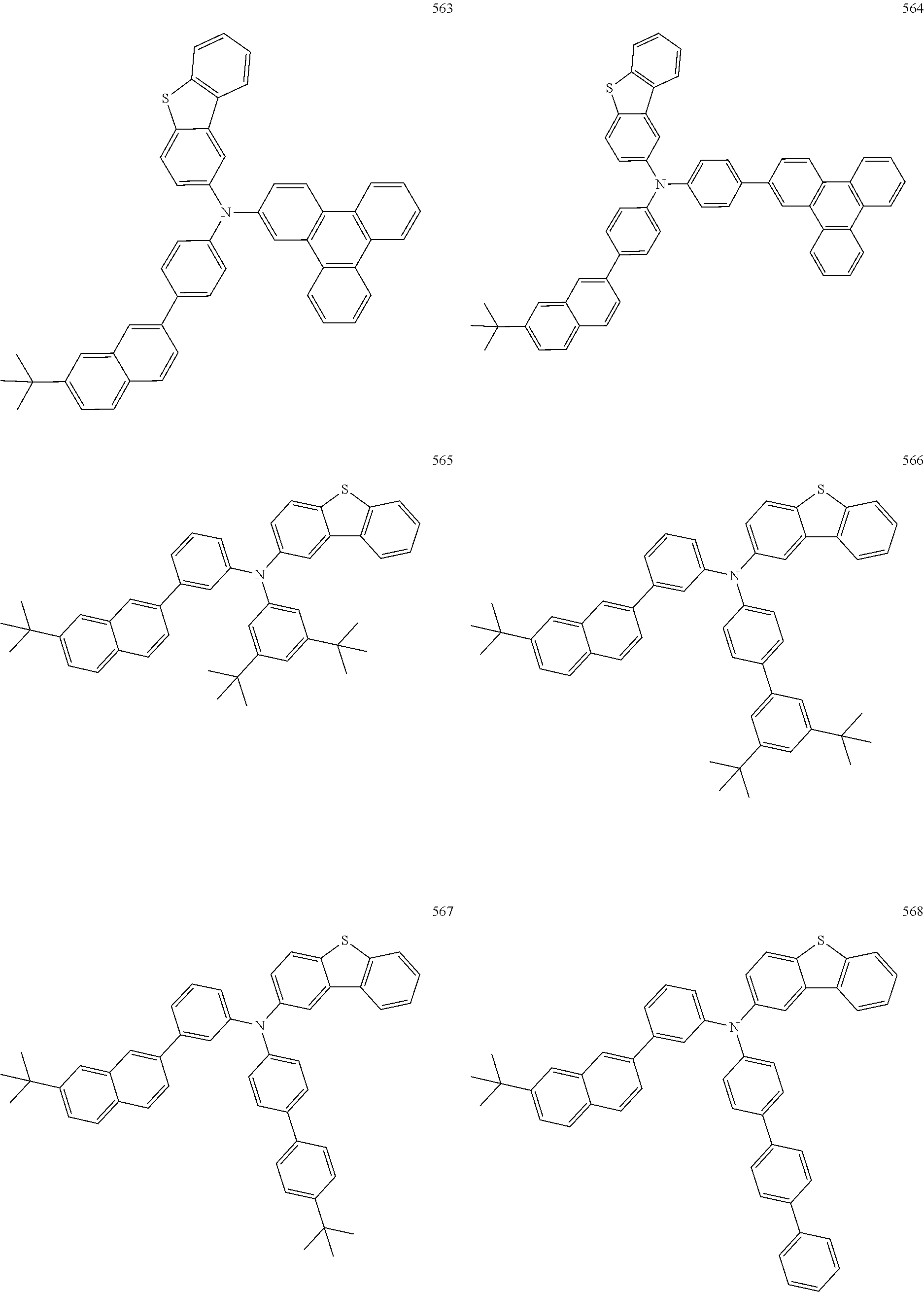

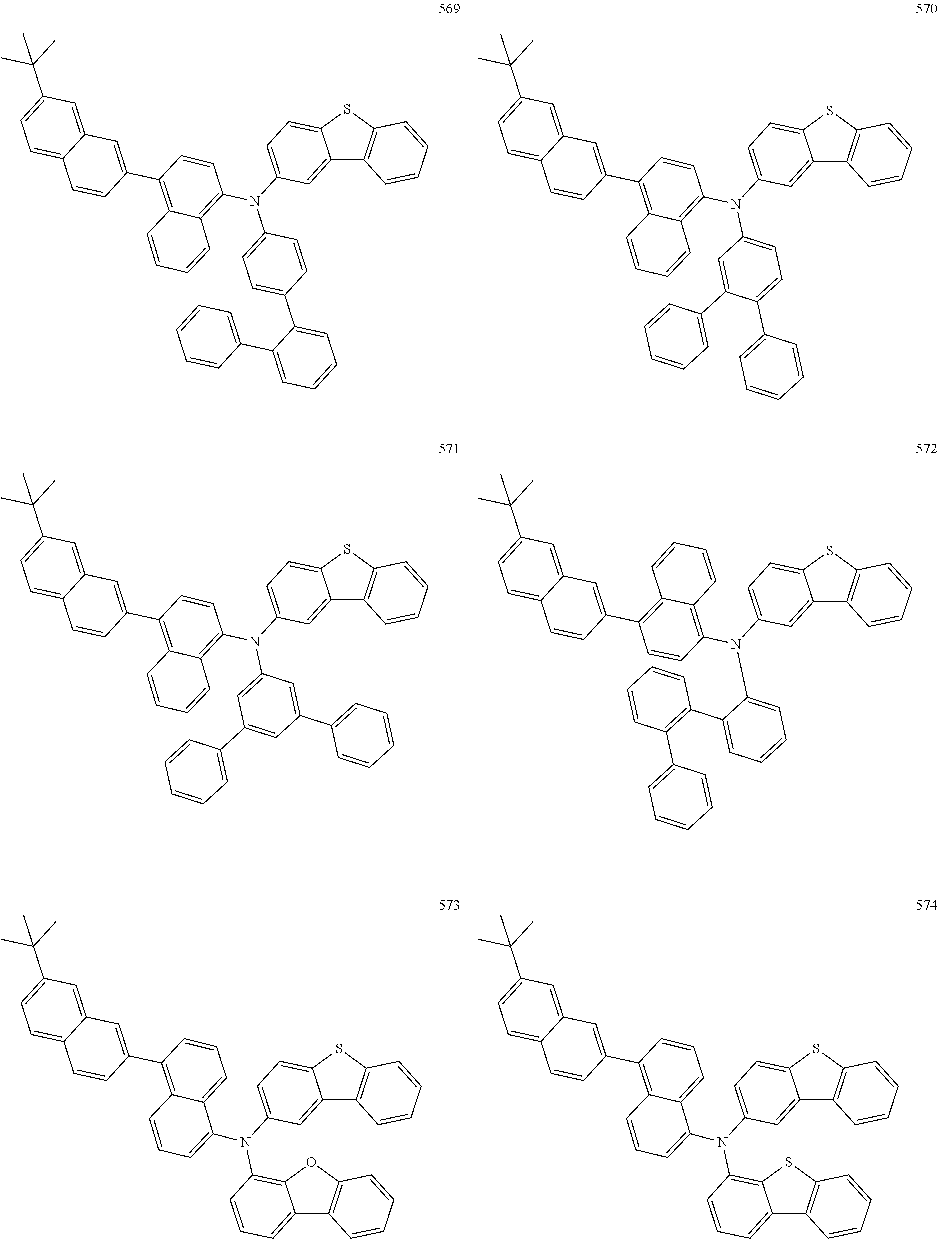

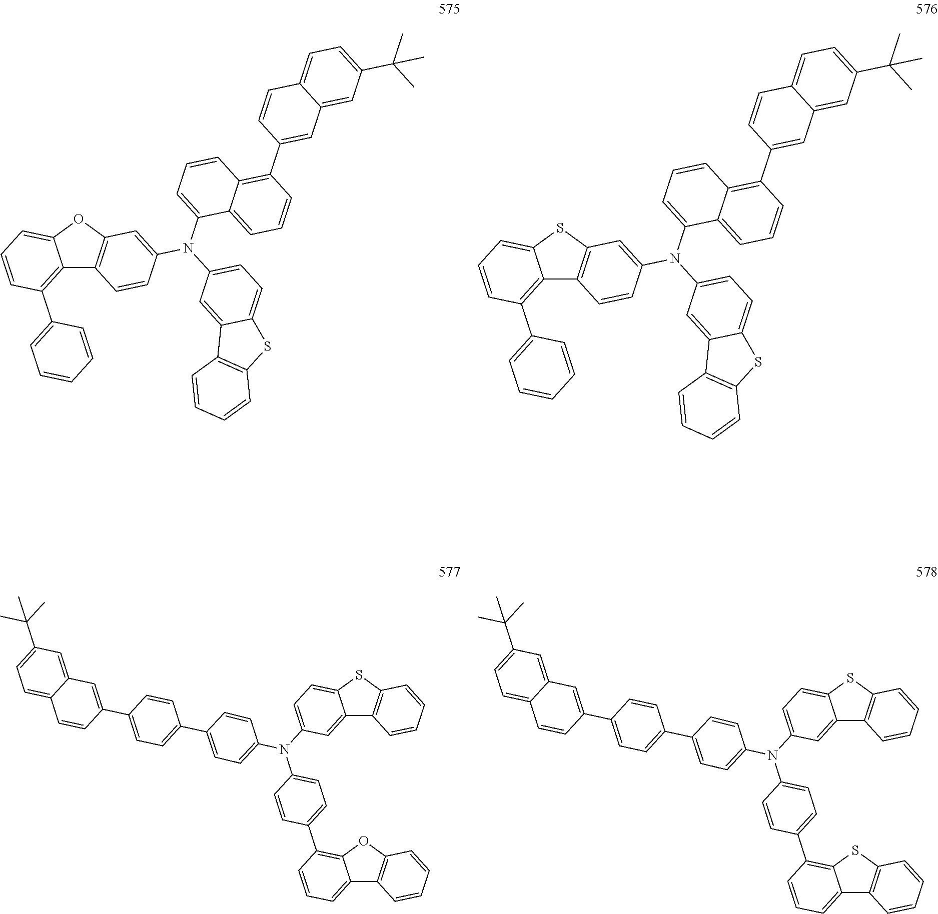 [SMILES: CC(C)(C)c1ccc2ccc(-c3cccc4c(N(c5ccc6c(c5)oc5cccc(-c7ccccc7)c56)c5ccc6sc7ccccc7c6c5)cccc34)cc2c1 ; CC(C)(C)c1ccc2ccc(-c3cccc4c(N(c5ccc6c(c5)sc5cccc(-c7ccccc7)c56)c5ccc6sc7ccccc7c6c5)cccc34)cc2c1 ; CC(C)(C)c1ccc2ccc(-c3ccc(-c4ccc(N(c5ccc(-c6cccc7c6oc6ccccc67)cc5)c5ccc6sc7ccccc7c6c5)cc4)cc3)cc2c1 ; CC(C)(C)c1ccc2ccc(-c3ccc(-c4ccc(N(c5ccc(-c6cccc7c6sc6ccccc67)cc5)c5ccc6sc7ccccc7c6c5)cc4)cc3)cc2c1]

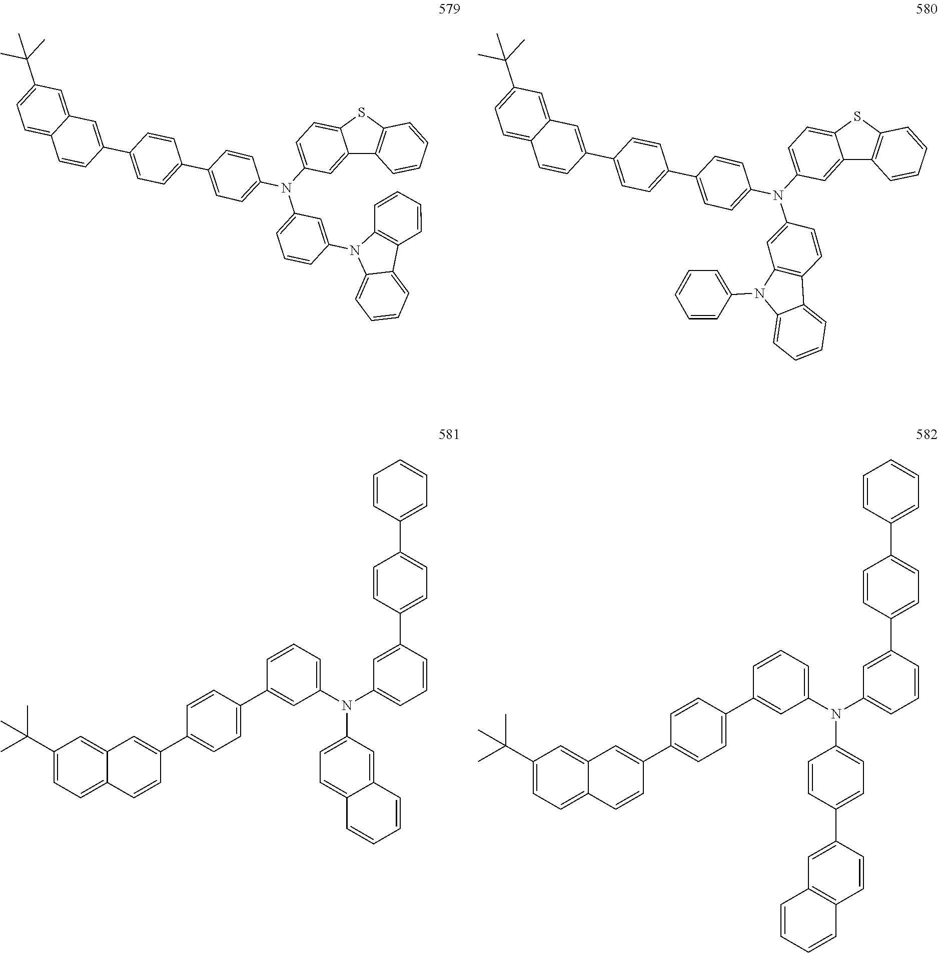 [SMILES: CC(C)(C)c1ccc2ccc(-c3ccc(-c4ccc(N(c5cccc(-n6c7ccccc7c7ccccc76)c5)c5ccc6sc7ccccc7c6c5)cc4)cc3)cc2c1 ; CC(C)(C)c1ccc2ccc(-c3ccc(-c4ccc(N(c5ccc6sc7ccccc7c6c5)c5ccc6c7ccccc7n(-c7ccccc7)c6c5)cc4)cc3)cc2c1 ; CC(C)(C)c1ccc2ccc(-c3ccc(-c4cccc(N(c5cccc(-c6ccc(-c7ccccc7)cc6)c5)c5ccc6ccccc6c5)c4)cc3)cc2c1 ; CC(C)(C)c1ccc2ccc(-c3ccc(-c4cccc(N(c5ccc(-c6ccc7ccccc7c6)cc5)c5cccc(-c6ccc(-c7ccccc7)cc6)c5)c4)cc3)cc2c1]

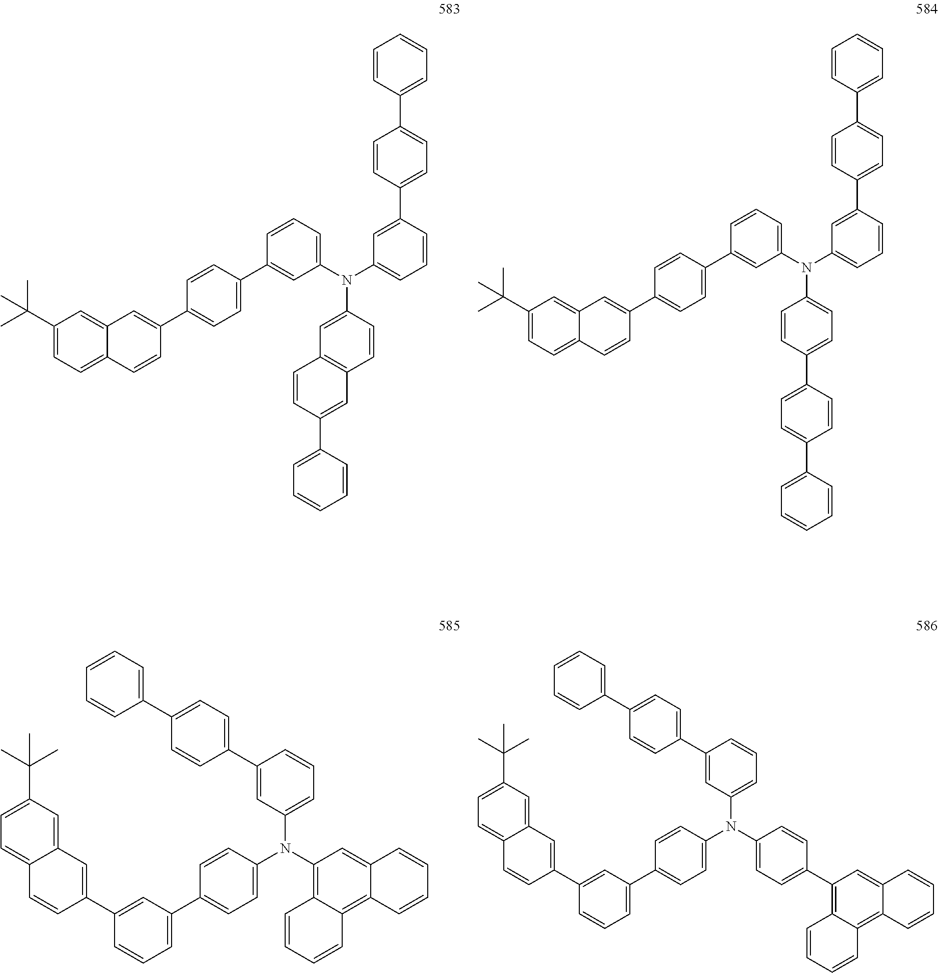 [SMILES: CC(C)(C)c1ccc2ccc(-c3ccc(-c4cccc(N(c5cccc(-c6ccc(-c7ccccc7)cc6)c5)c5ccc6cc(-c7ccccc7)ccc6c5)c4)cc3)cc2c1 ; CC(C)(C)c1ccc2ccc(-c3ccc(-c4cccc(N(c5ccc(-c6ccc(-c7ccccc7)cc6)cc5)c5cccc(-c6ccc(-c7ccccc7)cc6)c5)c4)cc3)cc2c1 ; CC(C)(C)c1ccc2ccc(-c3cccc(-c4ccc(N(c5cccc(-c6ccc(-c7ccccc7)cc6)c5)c5cc6ccccc6c6ccccc56)cc4)c3)cc2c1 ; CC(C)(C)c1ccc2ccc(-c3cccc(-c4ccc(N(c5ccc(-c6cc7ccccc7c7ccccc67)cc5)c5cccc(-c6ccc(-c7ccccc7)cc6)c5)cc4)c3)cc2c1]

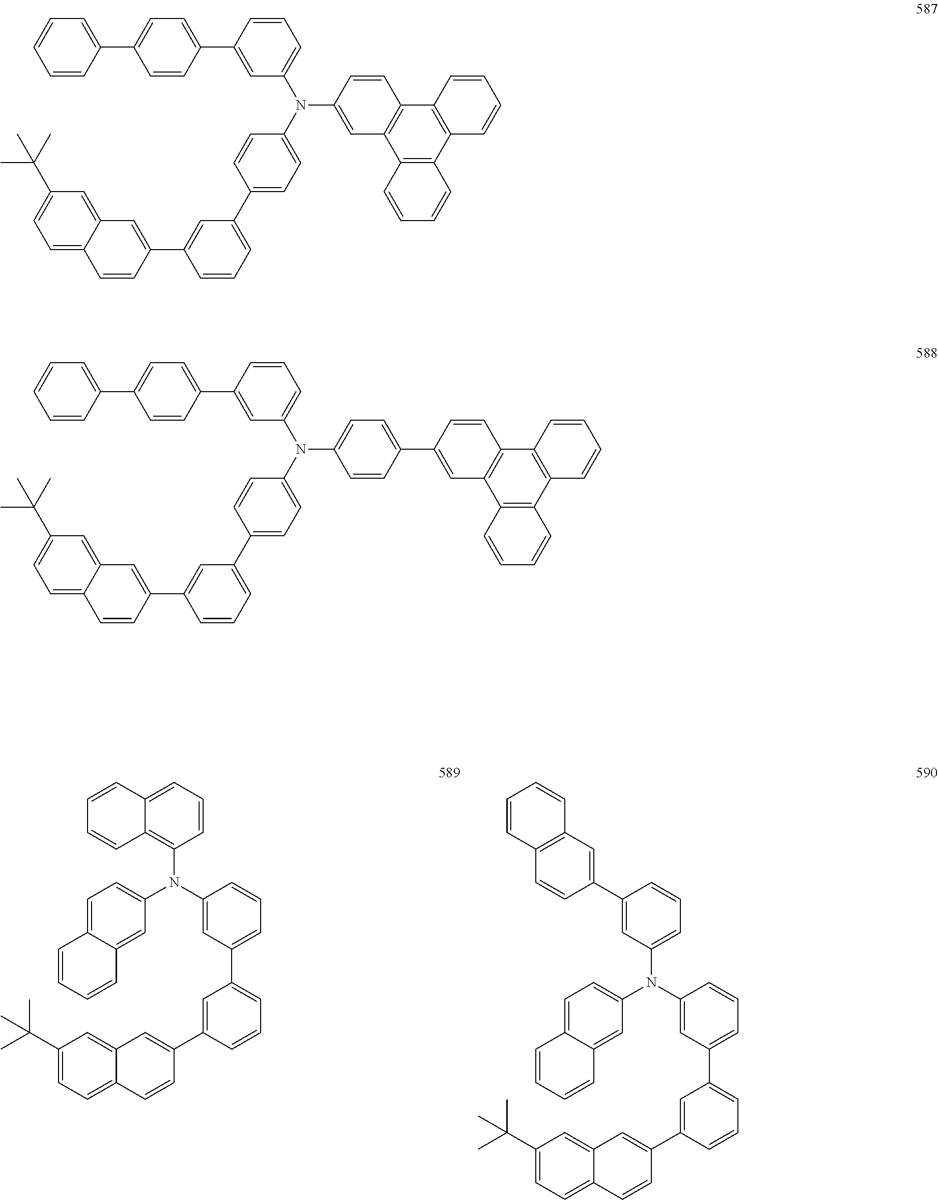 [SMILES: CC(C)(C)c1ccc2ccc(-c3cccc(-c4ccc(N(c5cccc(-c6ccc(-c7ccccc7)cc6)c5)c5ccc6c7ccccc7c7ccccc7c6c5)cc4)c3)cc2c1 ; CC(C)(C)c1ccc2ccc(-c3cccc(-c4ccc(N(c5ccc(-c6ccc7c8ccccc8c8ccccc8c7c6)cc5)c5cccc(-c6ccc(-c7ccccc7)cc6)c5)cc4)c3)cc2c1 ; CC(C)(C)c1ccc2ccc(-c3cccc(-c4cccc(N(c5ccc6ccccc6c5)c5cccc6ccccc56)c4)c3)cc2c1 ; CC(C)(C)c1ccc2ccc(-c3cccc(-c4cccc(N(c5cccc(-c6ccc7ccccc7c6)c5)c5ccc6ccccc6c5)c4)c3)cc2c1]

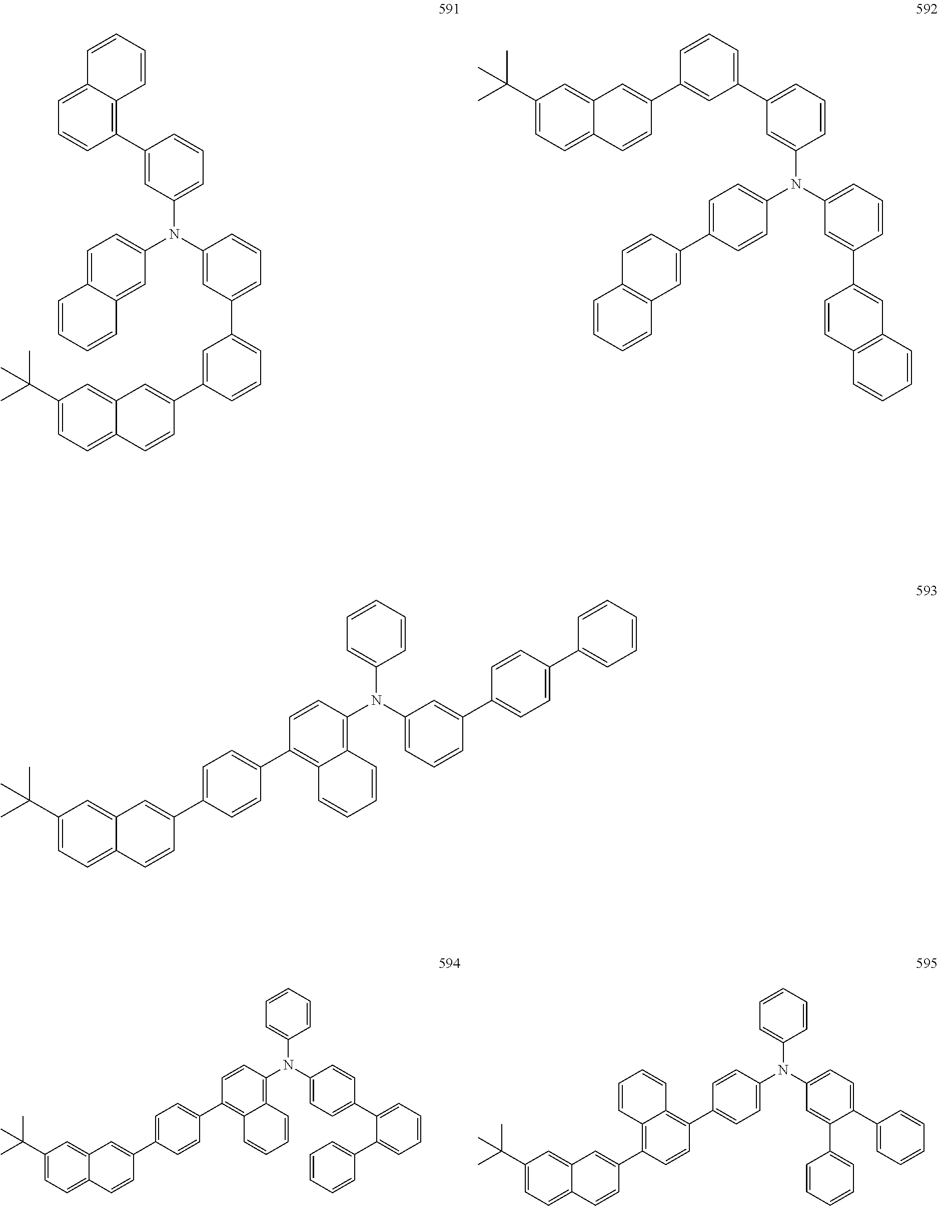 [SMILES: CC(C)(C)c1ccc2ccc(-c3cccc(-c4cccc(N(c5cccc(-c6cccc7ccccc67)c5)c5ccc6ccccc6c5)c4)c3)cc2c1 ; CC(C)(C)c1ccc2ccc(-c3cccc(-c4cccc(N(c5ccc(-c6ccc7ccccc7c6)cc5)c5cccc(-c6ccc7ccccc7c6)c5)c4)c3)cc2c1 ; CC(C)(C)c1ccc2ccc(-c3ccc(-c4ccc(N(c5ccccc5)c5cccc(-c6ccc(-c7ccccc7)cc6)c5)c5ccccc45)cc3)cc2c1 ; CC(C)(C)c1ccc2ccc(-c3ccc(-c4ccc(N(c5ccccc5)c5ccc(-c6ccccc6-c6ccccc6)cc5)c5ccccc45)cc3)cc2c1 ; CC(C)(C)c1ccc2ccc(-c3ccc(-c4ccc(N(c5ccccc5)c5ccc(-c6ccccc6)c(-c6ccccc6)c5)cc4)c4ccccc34)cc2c1]

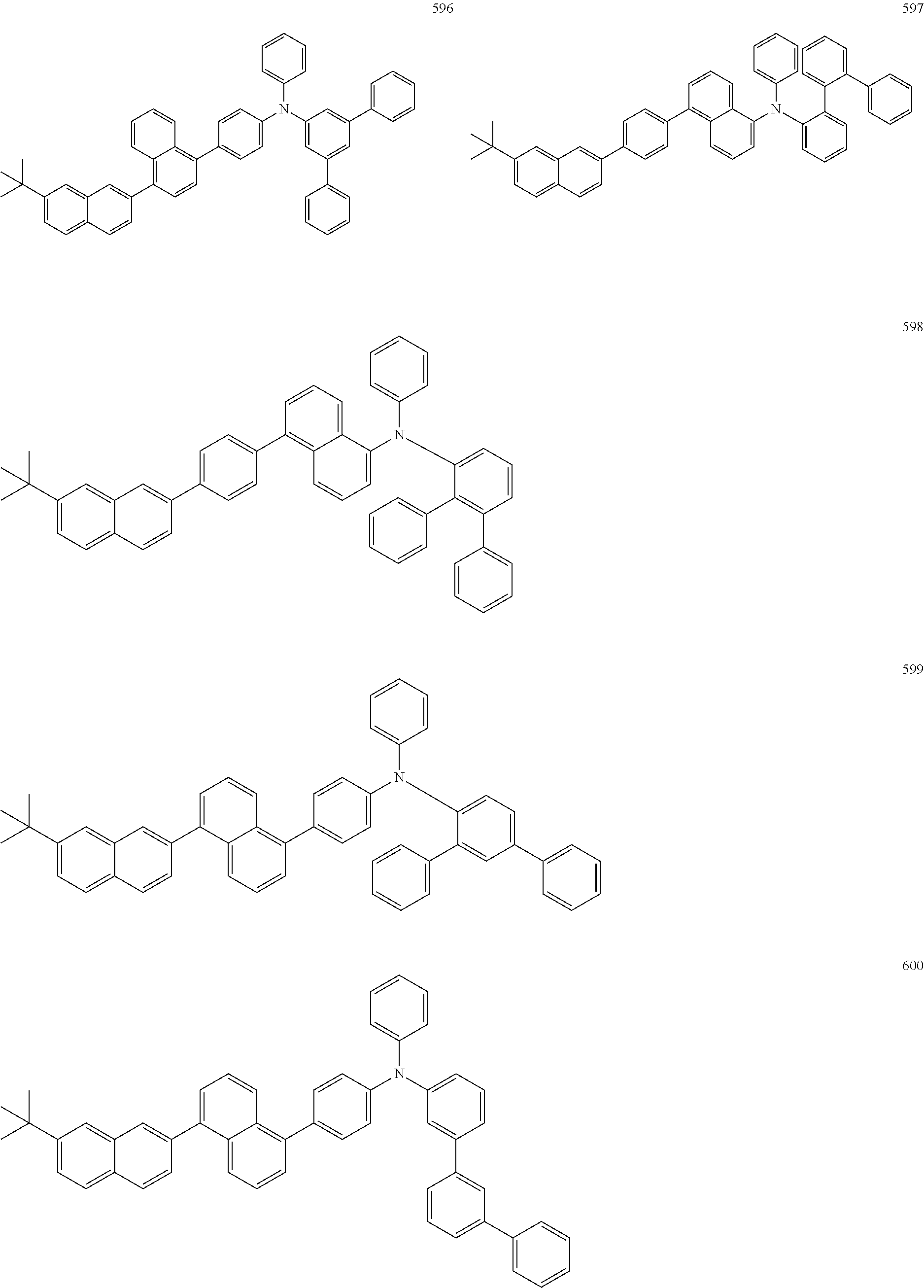 [SMILES: CC(C)(C)c1ccc2ccc(-c3ccc(-c4ccc(N(c5ccccc5)c5cc(-c6ccccc6)cc(-c6ccccc6)c5)cc4)c4ccccc34)cc2c1 ; CC(C)(C)c1ccc2ccc(-c3ccc(-c4cccc5c(N(c6ccccc6)c6ccccc6-c6ccccc6-c6ccccc6)cccc45)cc3)cc2c1 ; CC(C)(C)c1ccc2ccc(-c3ccc(-c4cccc5c(N(c6ccccc6)c6cccc(-c7ccccc7)c6-c6ccccc6)cccc45)cc3)cc2c1 ; CC(C)(C)c1ccc2ccc(-c3cccc4c(-c5ccc(N(c6ccccc6)c6ccc(-c7ccccc7)cc6-c6ccccc6)cc5)cccc34)cc2c1 ; CC(C)(C)c1ccc2ccc(-c3cccc4c(-c5ccc(N(c6ccccc6)c6cccc(-c7cccc(-c8ccccc8)c7)c6)cc5)cccc34)cc2c1]

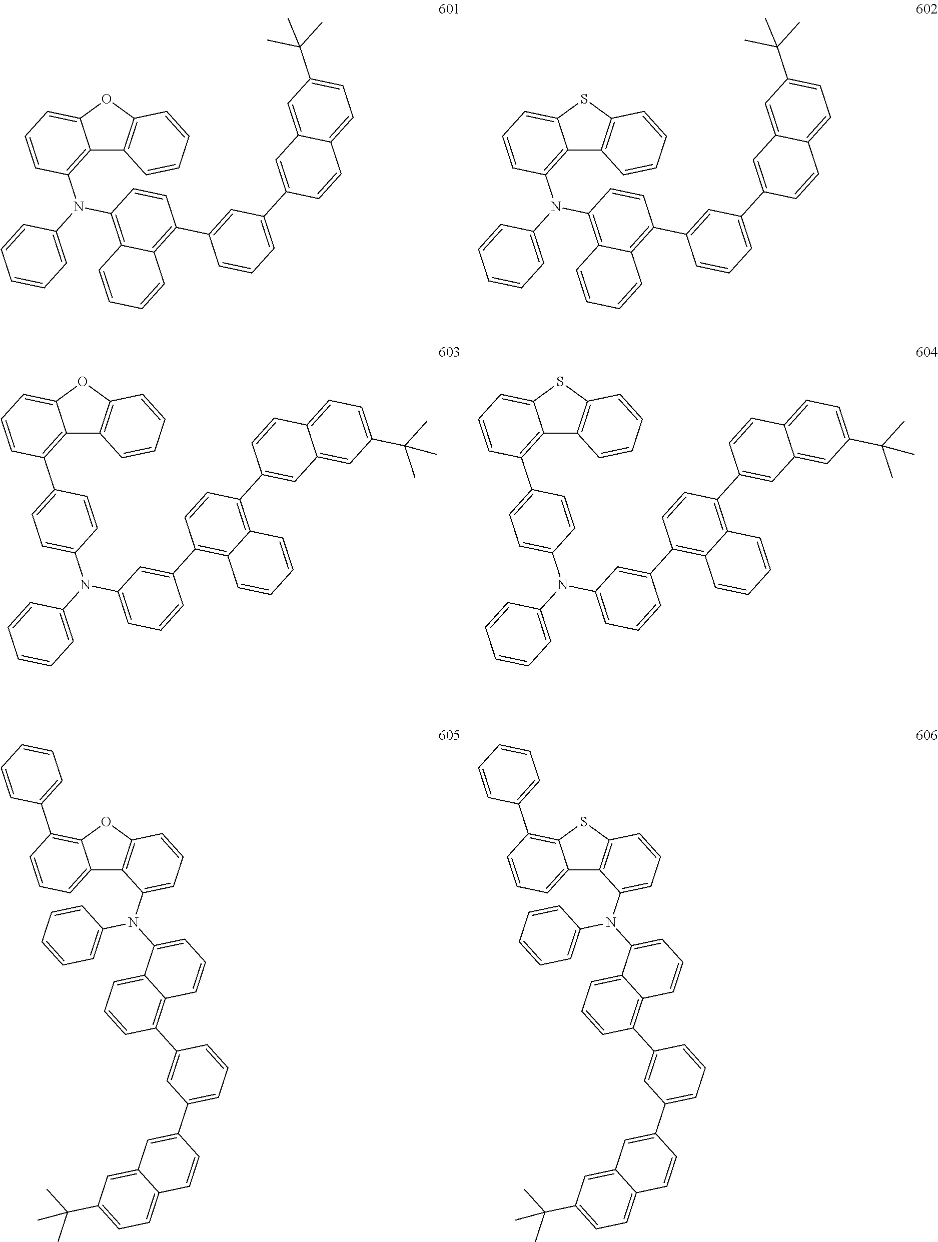

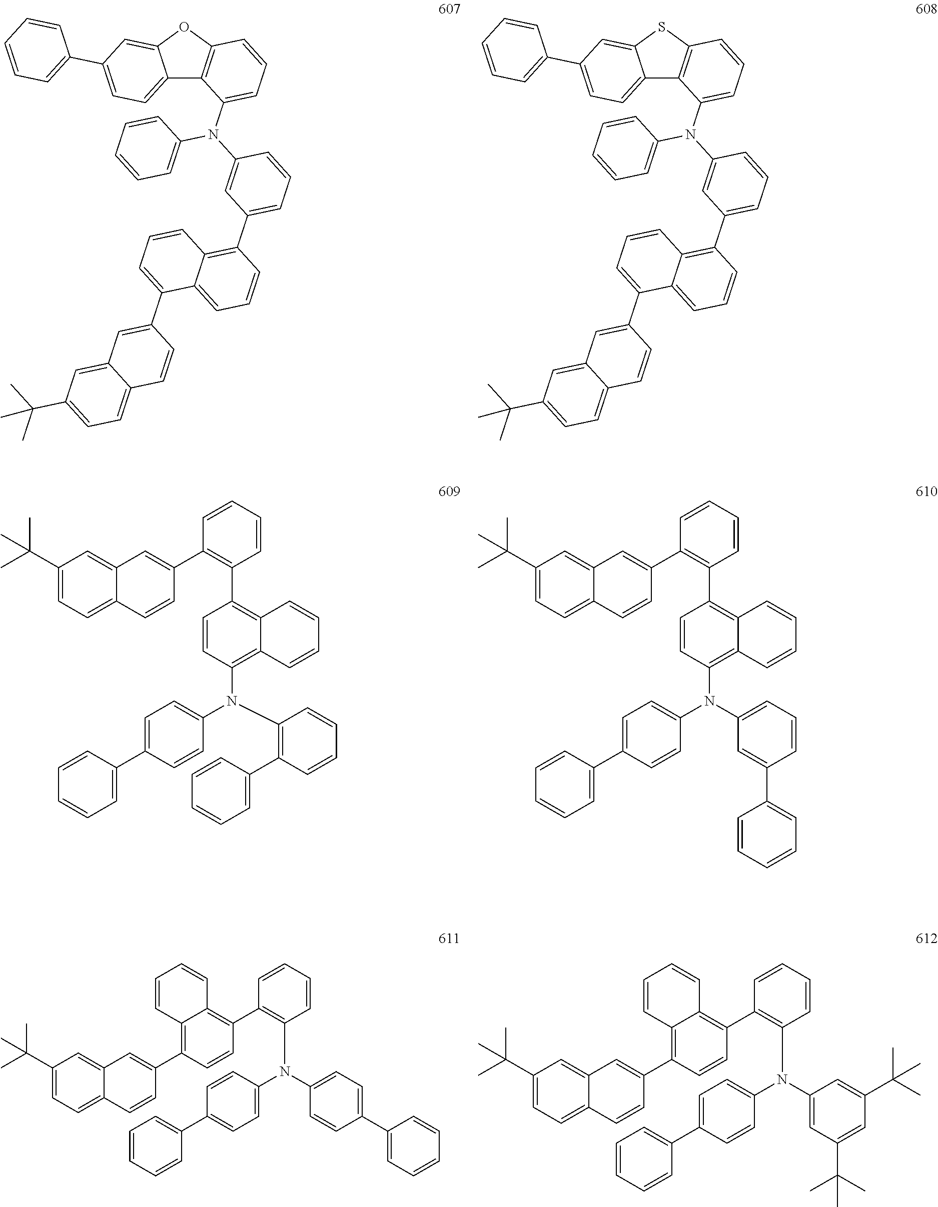

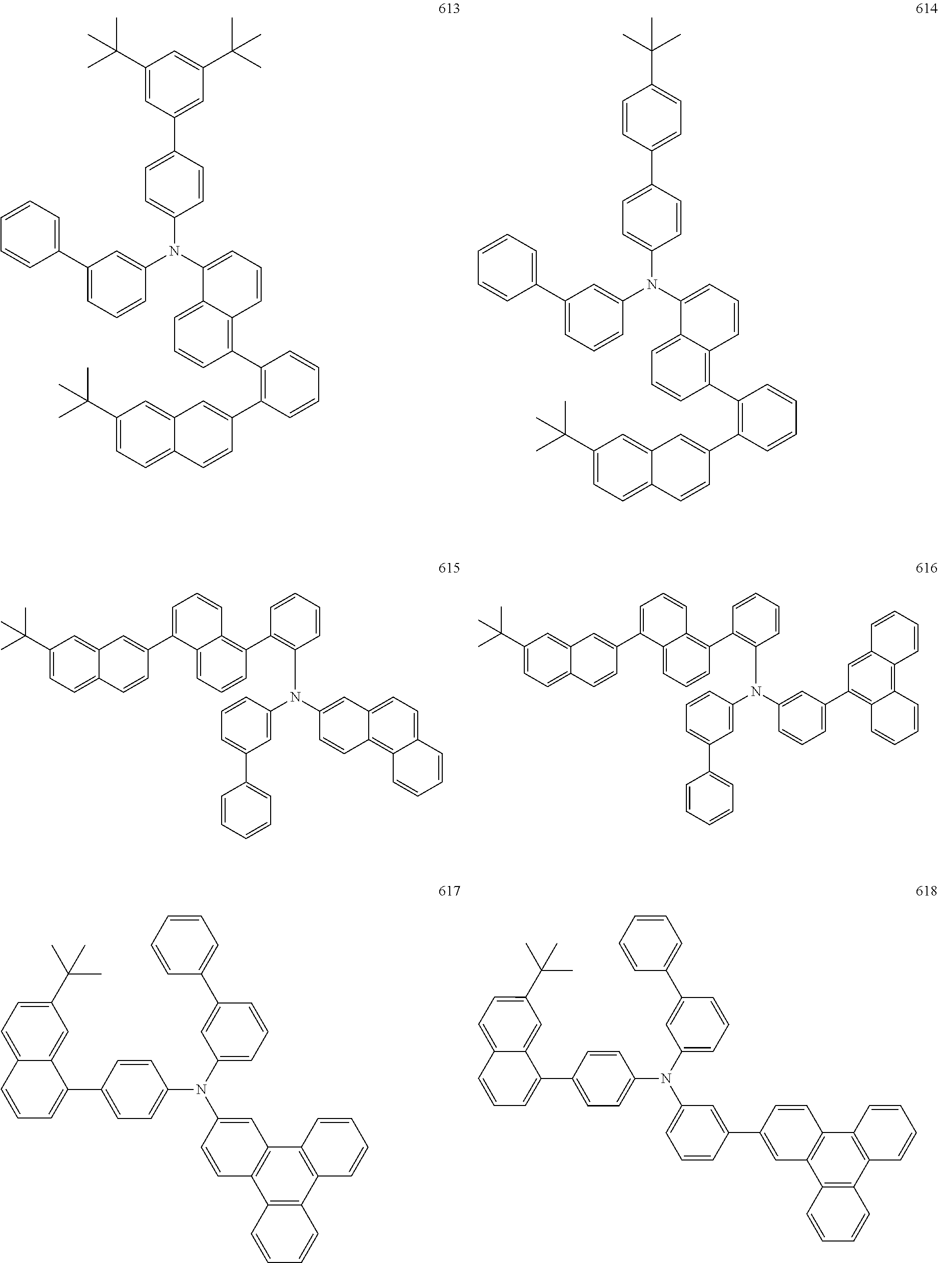

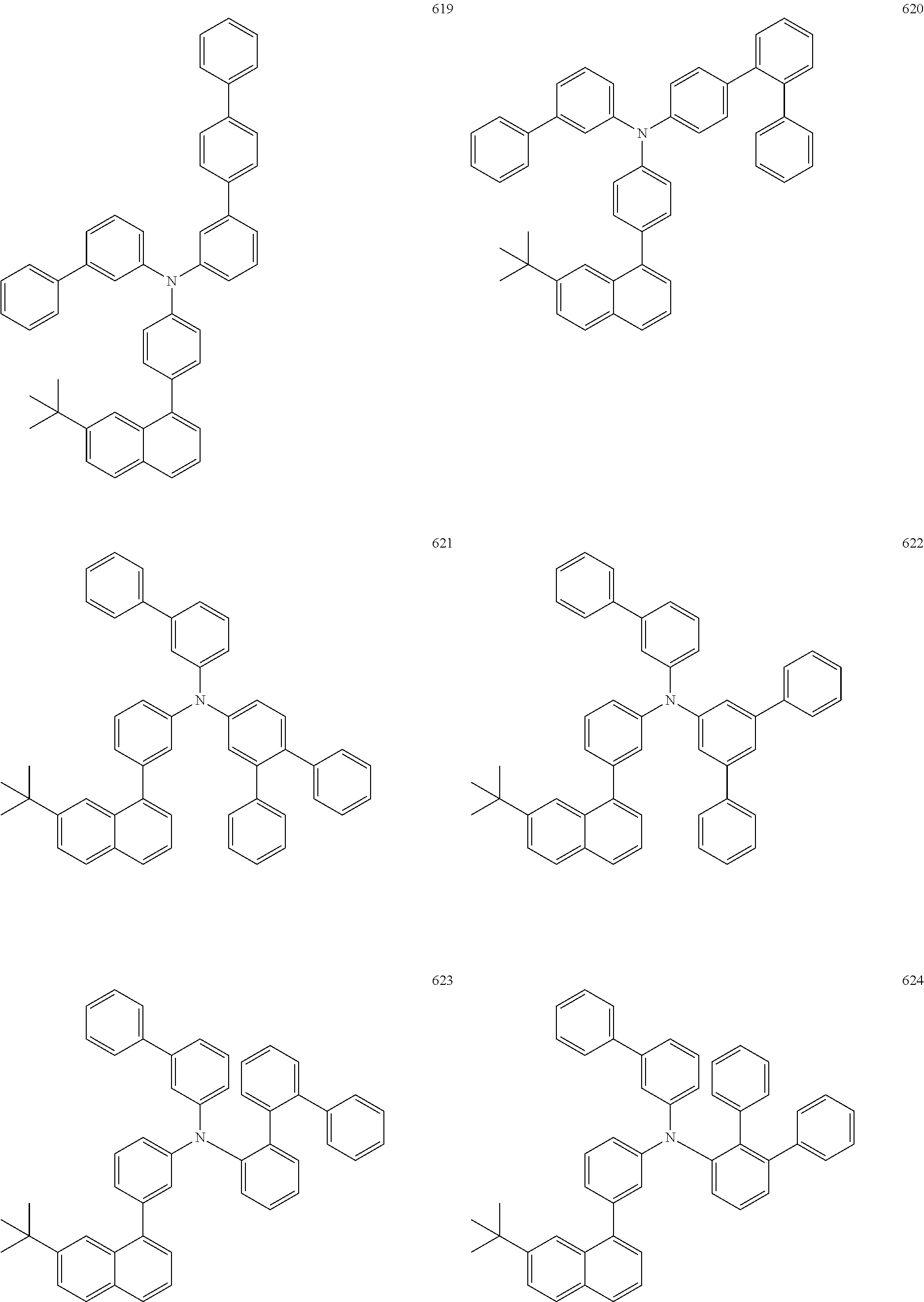

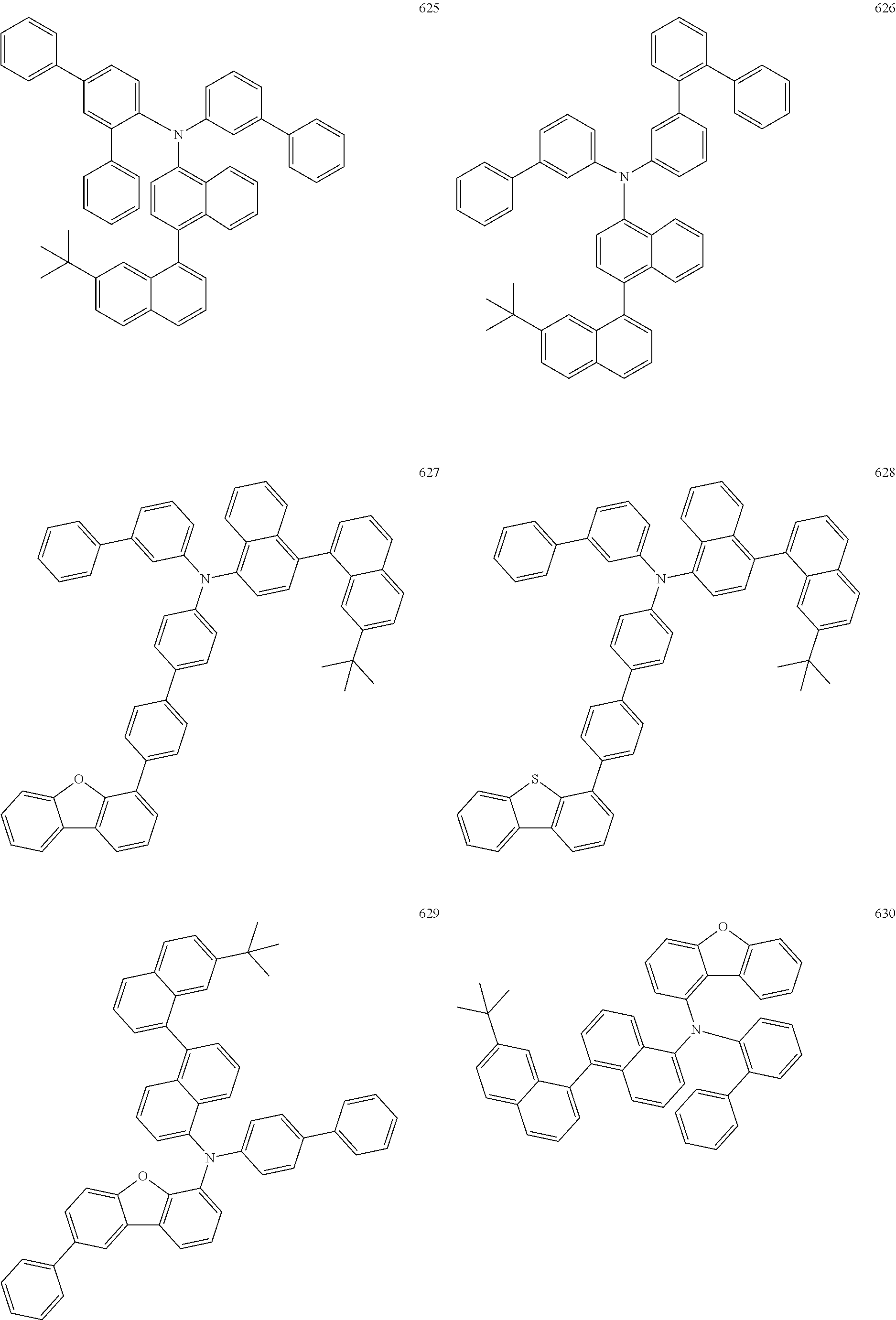

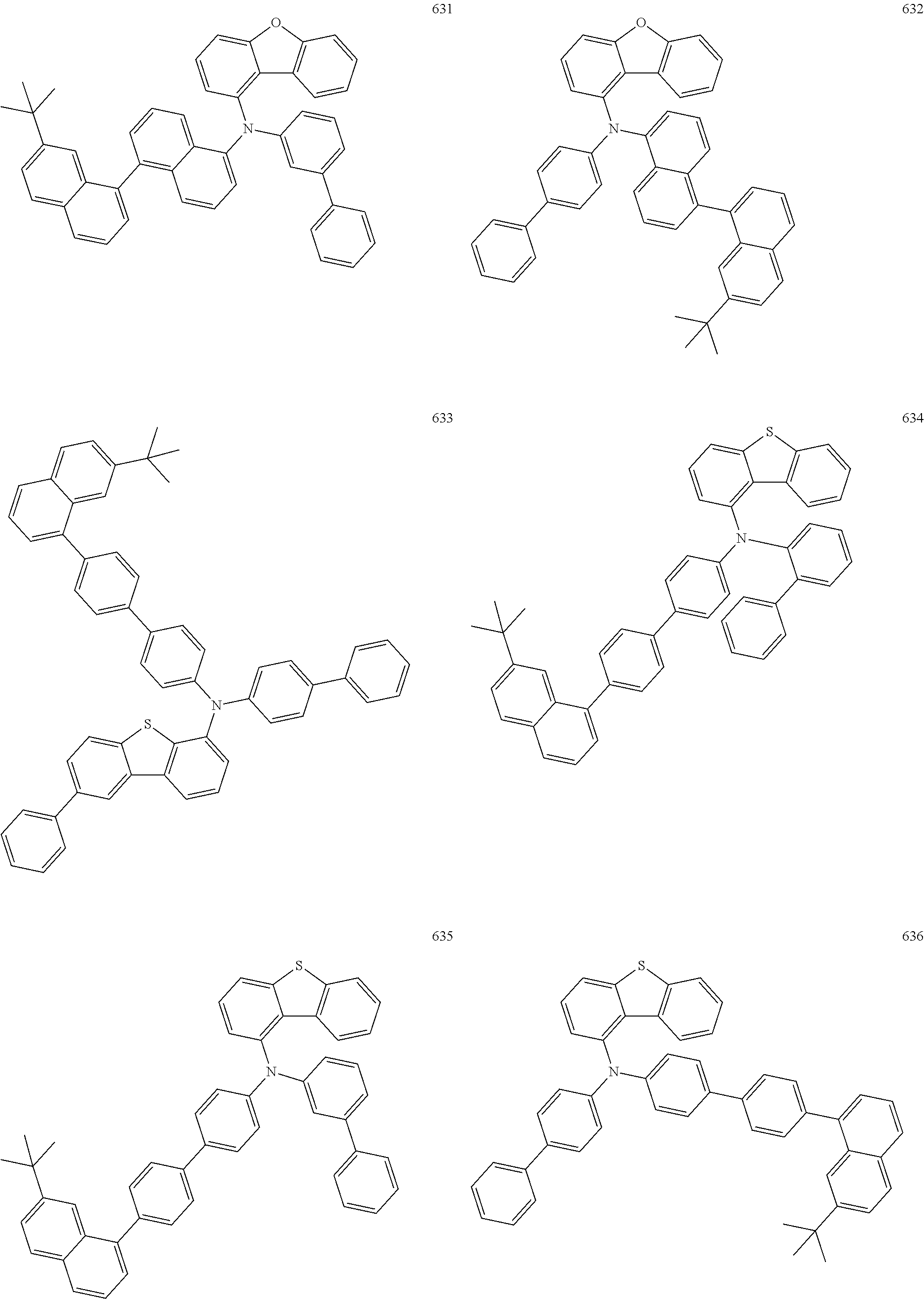

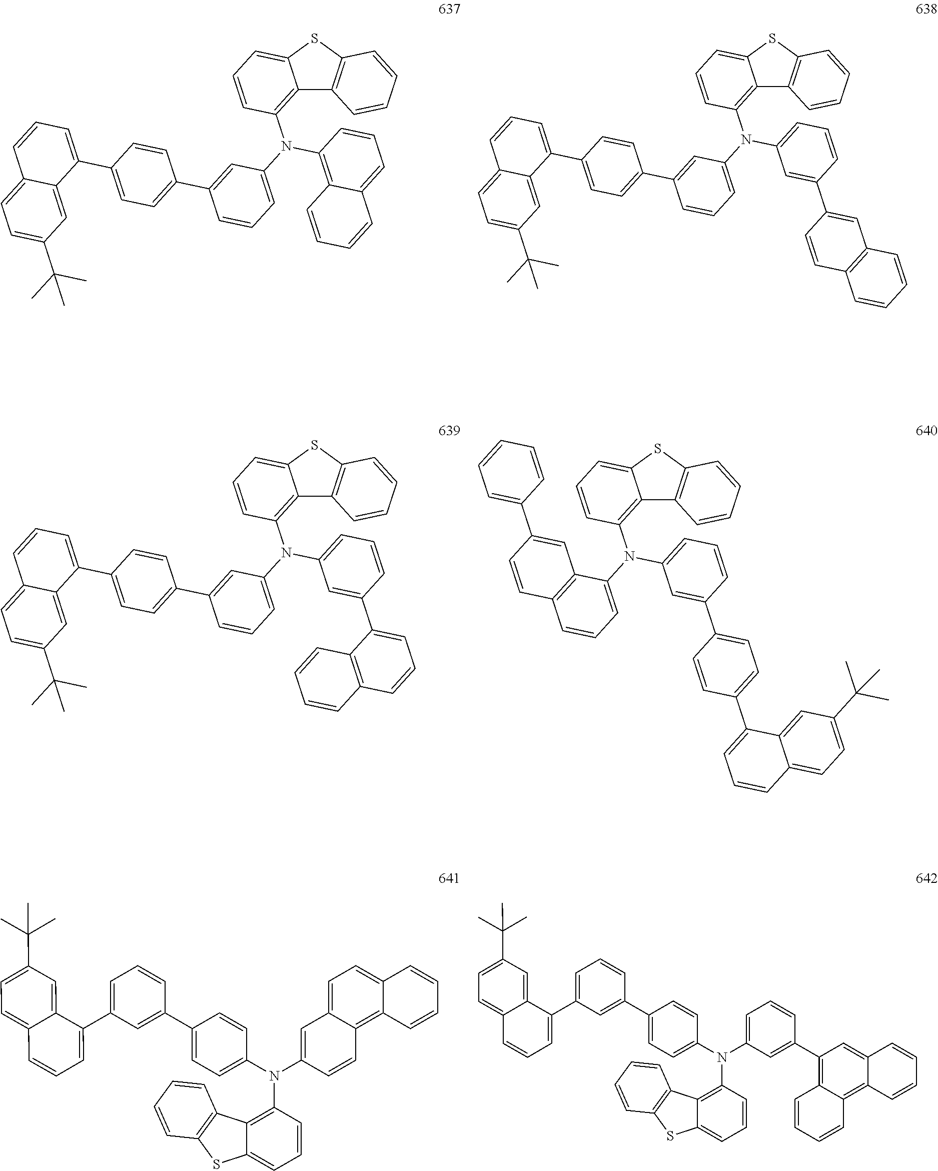

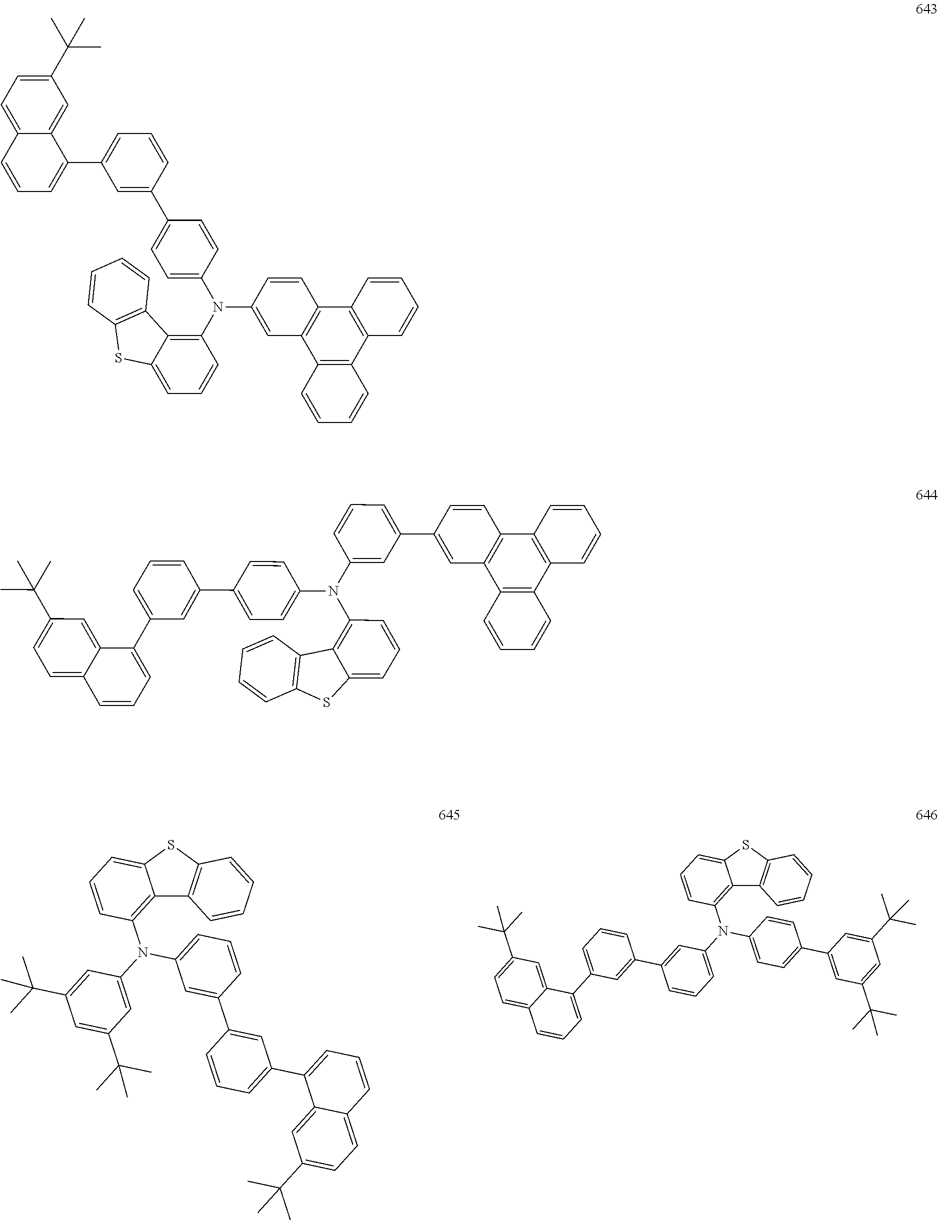 [SMILES: CC(C)(C)c1ccc2cccc(-c3cccc(-c4ccc(N(c5ccc6c7ccccc7c7ccccc7c6c5)c5cccc6sc7ccccc7c56)cc4)c3)c2c1 ; CC(C)(C)c1ccc2cccc(-c3cccc(-c4ccc(N(c5cccc(-c6ccc7c8ccccc8c8ccccc8c7c6)c5)c5cccc6sc7ccccc7c56)cc4)c3)c2c1 ; CC(C)(C)c1cc(N(c2cccc(-c3cccc(-c4cccc5ccc(C(C)(C)C)cc45)c3)c2)c2cccc3sc4ccccc4c23)cc(C(C)(C)C)c1 ; CC(C)(C)c1cc(-c2ccc(N(c3cccc(-c4cccc(-c5cccc6ccc(C(C)(C)C)cc56)c4)c3)c3cccc4sc5ccccc5c34)cc2)cc(C(C)(C)C)c1]

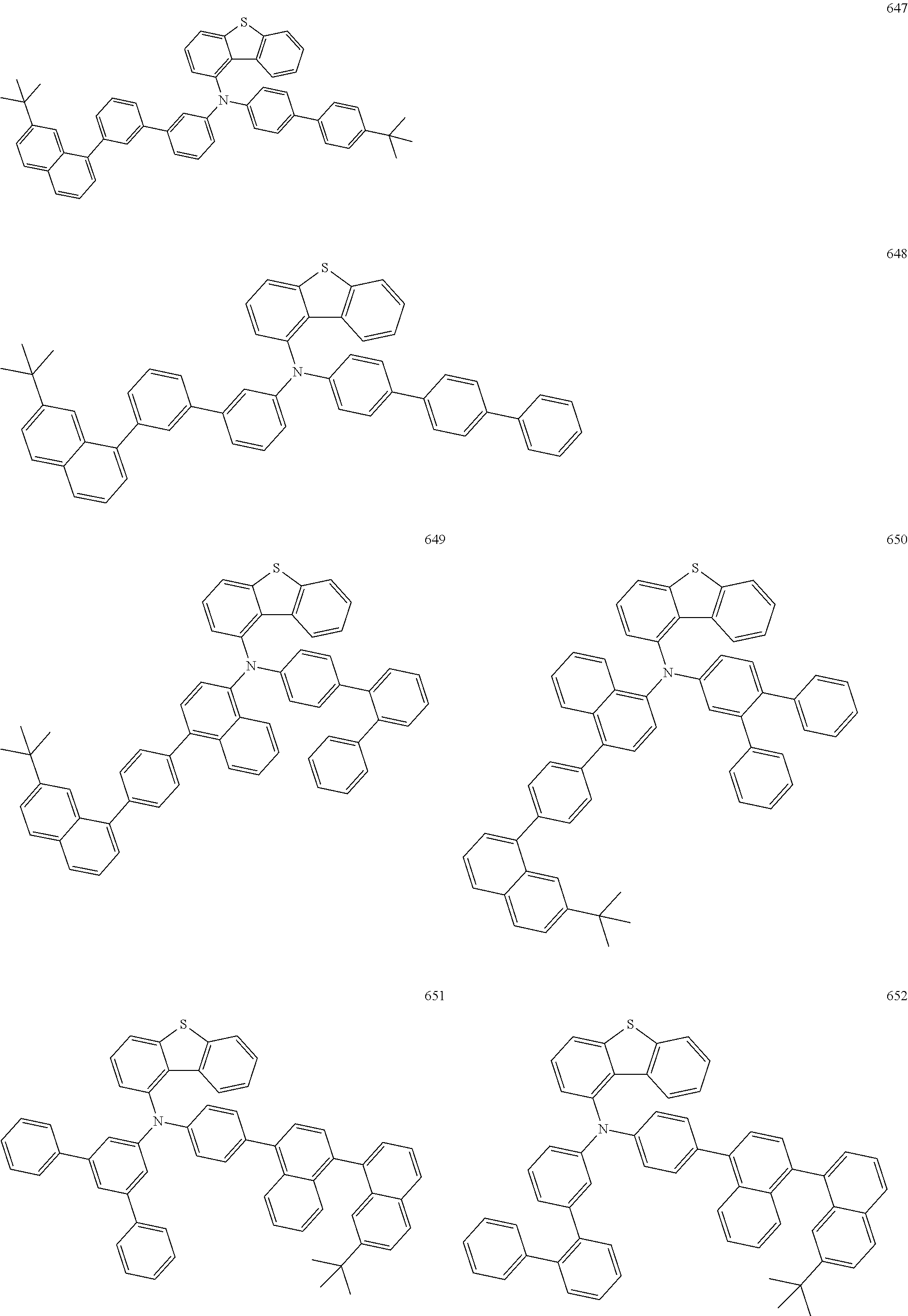

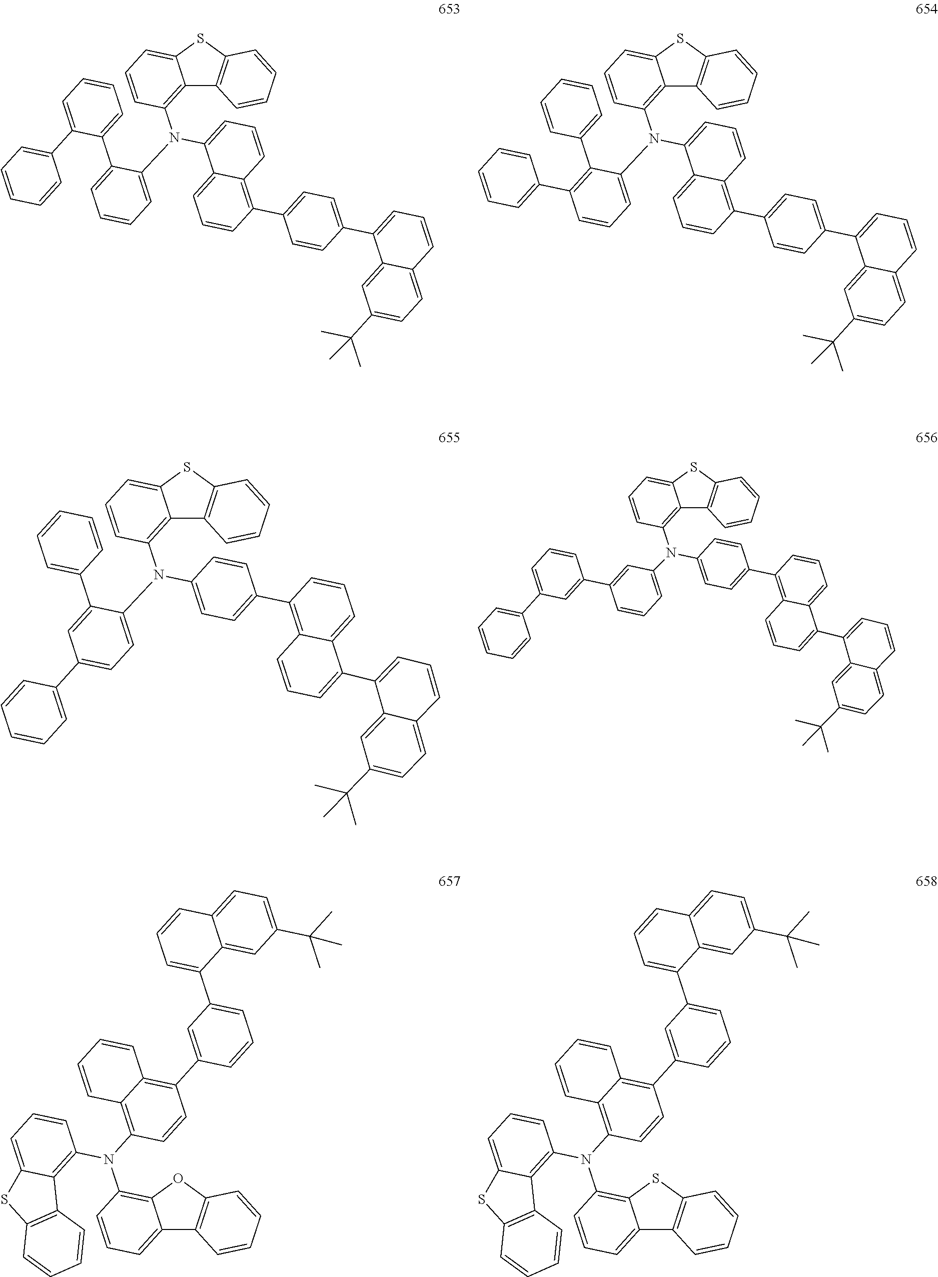

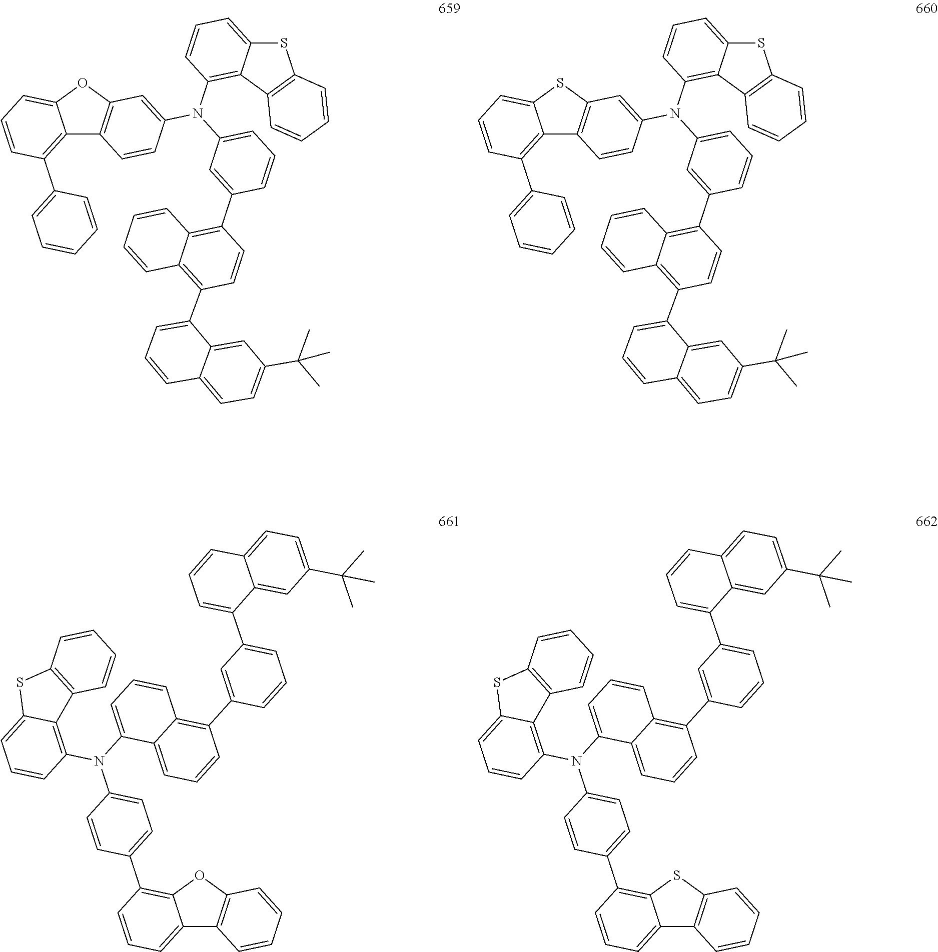 [SMILES: CC(C)(C)c1ccc2cccc(-c3ccc(-c4cccc(N(c5ccc6c(c5)oc5cccc(-c7ccccc7)c56)c5cccc6sc7ccccc7c56)c4)c4ccccc34)c2c1 ; CC(C)(C)c1ccc2cccc(-c3ccc(-c4cccc(N(c5ccc6c(c5)sc5cccc(-c7ccccc7)c56)c5cccc6sc7ccccc7c56)c4)c4ccccc34)c2c1 ; CC(C)(C)c1ccc2cccc(-c3cccc(-c4cccc5c(N(c6ccc(-c7cccc8c7oc7ccccc78)cc6)c6cccc7sc8ccccc8c67)cccc45)c3)c2c1 ; CC(C)(C)c1ccc2cccc(-c3cccc(-c4cccc5c(N(c6ccc(-c7cccc8c7sc7ccccc78)cc6)c6cccc7sc8ccccc8c67)cccc45)c3)c2c1]

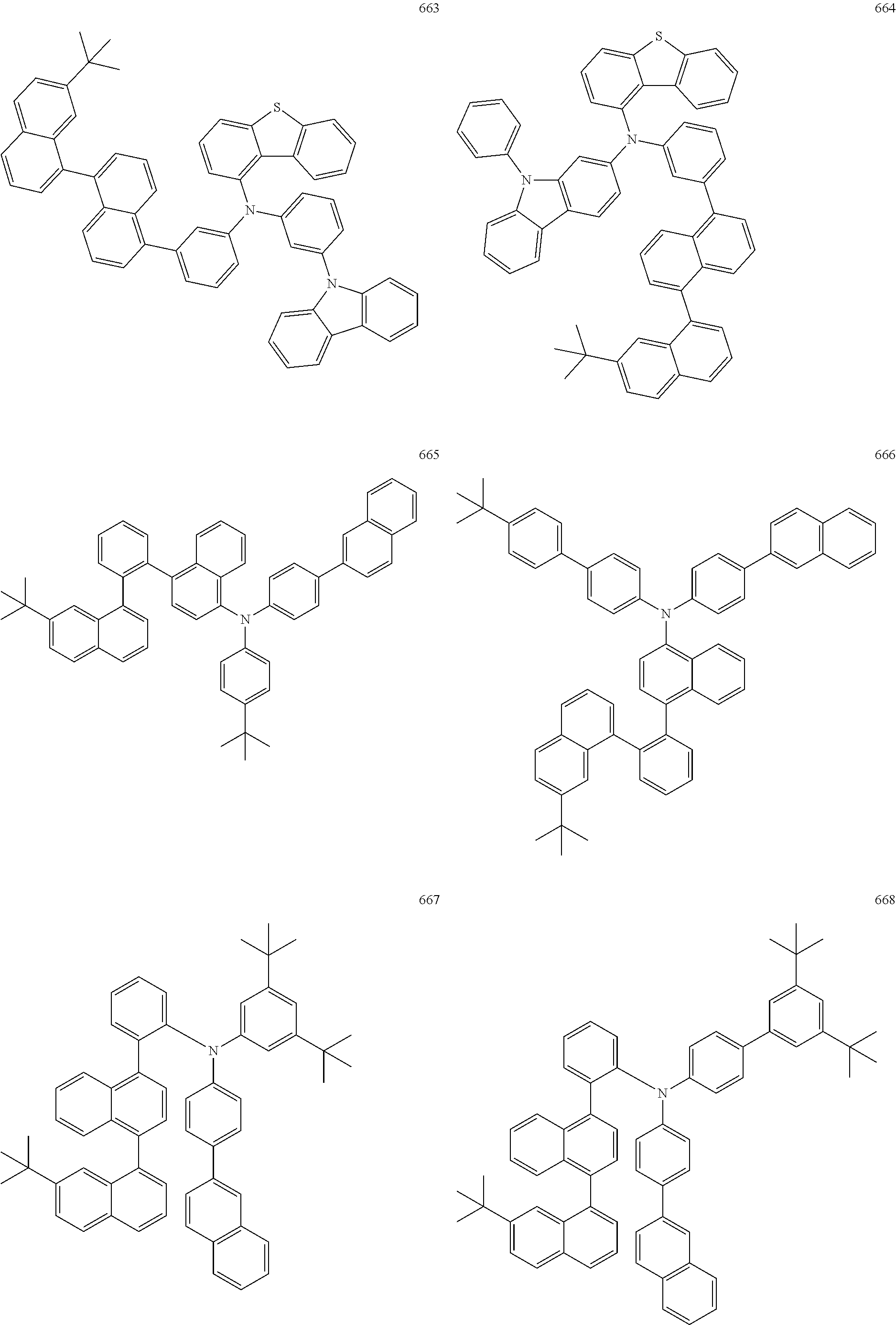

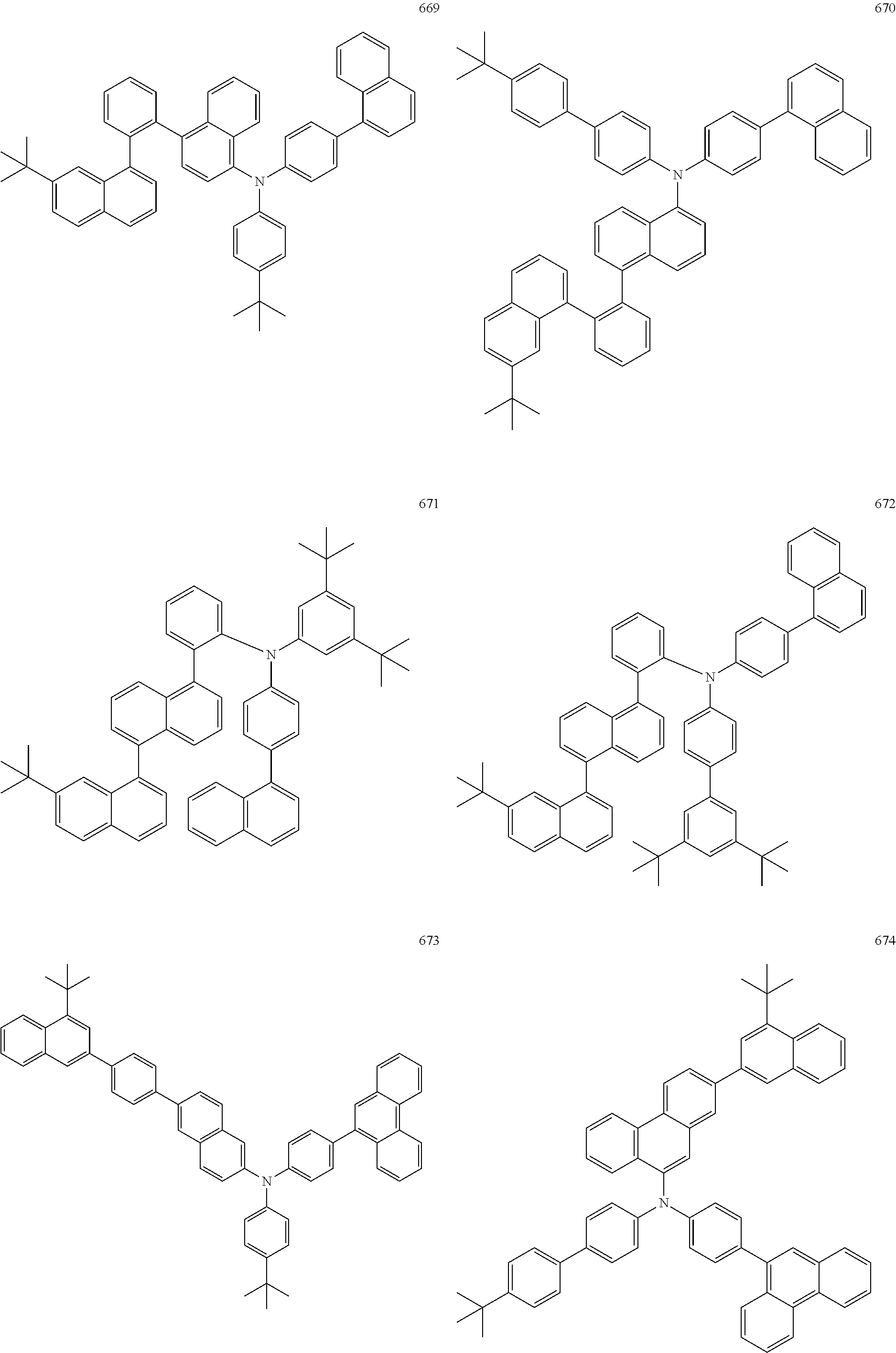

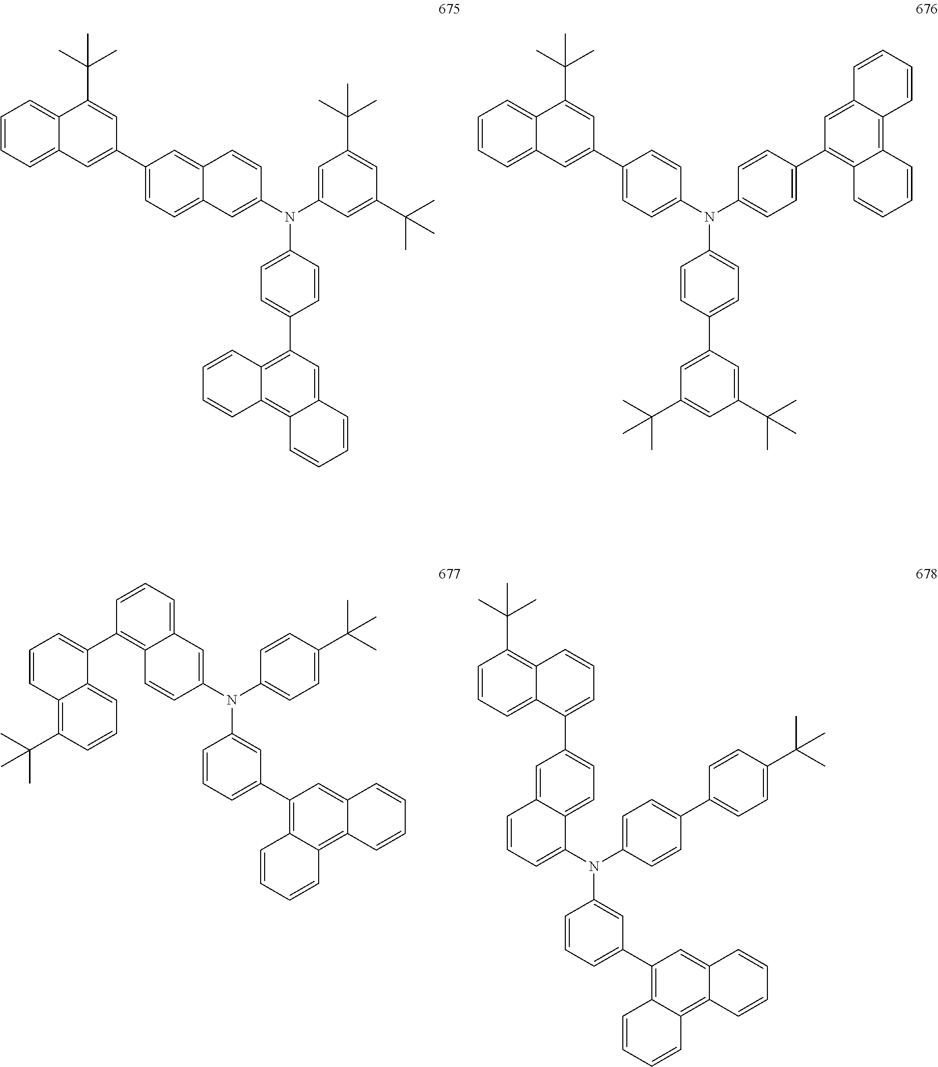 [SMILES: CC(C)(C)c1cc(N(c2ccc(-c3cc4ccccc4c4ccccc34)cc2)c2ccc3cc(-c4cc(C(C)(C)C)c5ccccc5c4)ccc3c2)cc(C(C)(C)C)c1 ; CC(C)(C)c1cc(-c2ccc(N(c3ccc(-c4cc(C(C)(C)C)c5ccccc5c4)cc3)c3ccc(-c4cc5ccccc5c5ccccc45)cc3)cc2)cc(C(C)(C)C)c1 ; CC(C)(C)c1ccc(N(c2cccc(-c3cc4ccccc4c4ccccc34)c2)c2ccc3c(-c4cccc5c(C(C)(C)C)cccc45)cccc3c2)cc1 ; CC(C)(C)c1ccc(-c2ccc(N(c3cccc(-c4cc5ccccc5c5ccccc45)c3)c3cccc4cc(-c5cccc6c(C(C)(C)C)cccc56)ccc34)cc2)cc1]

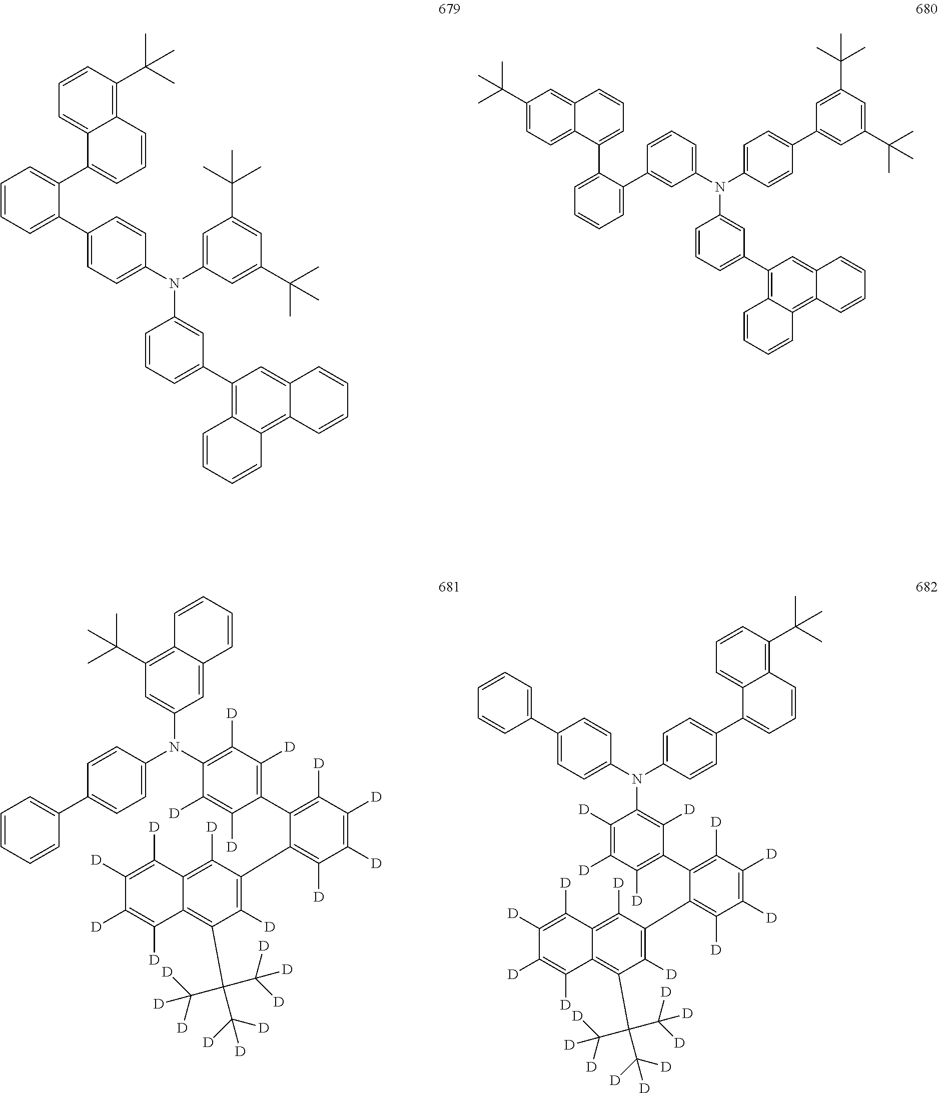

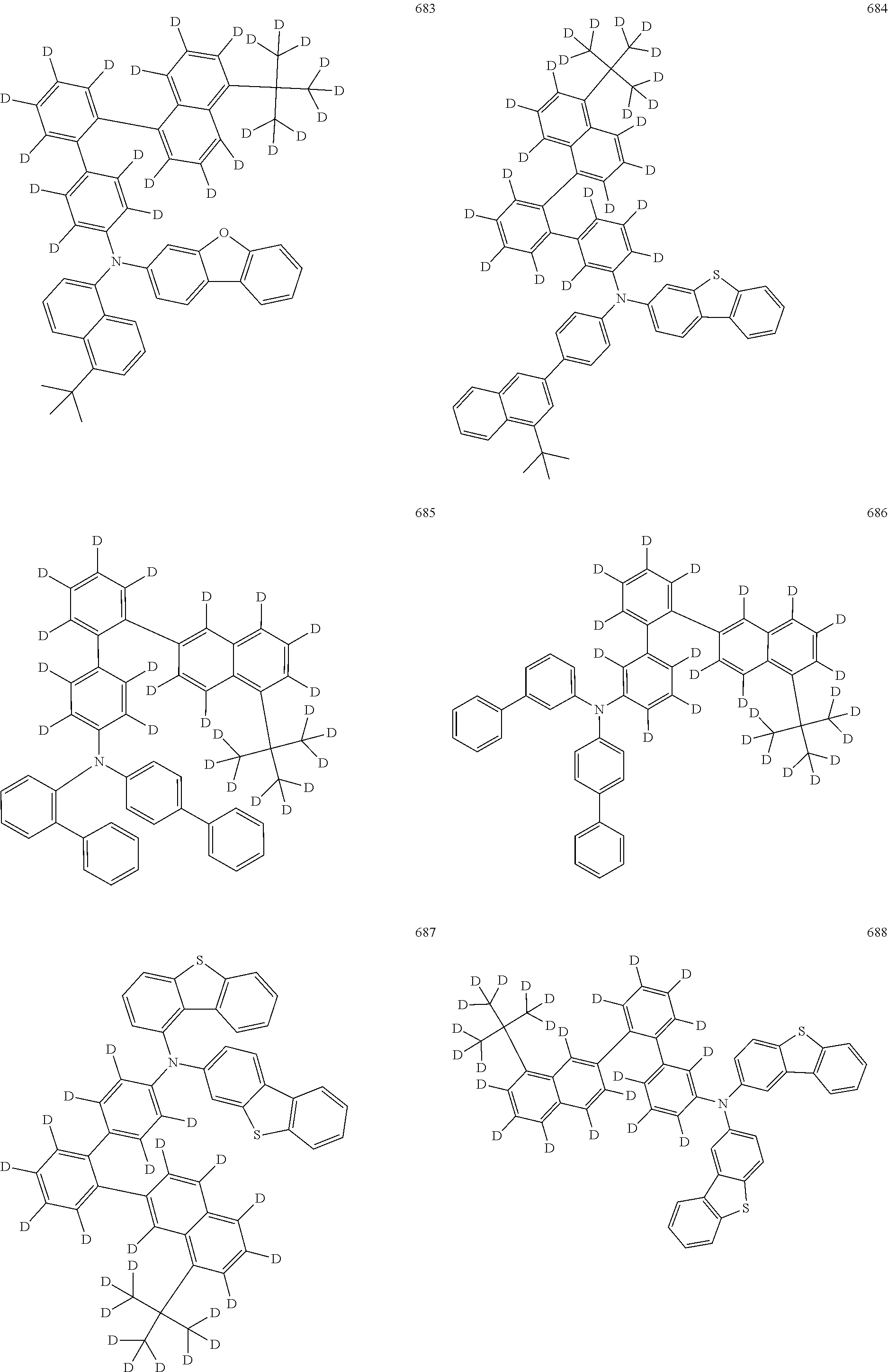

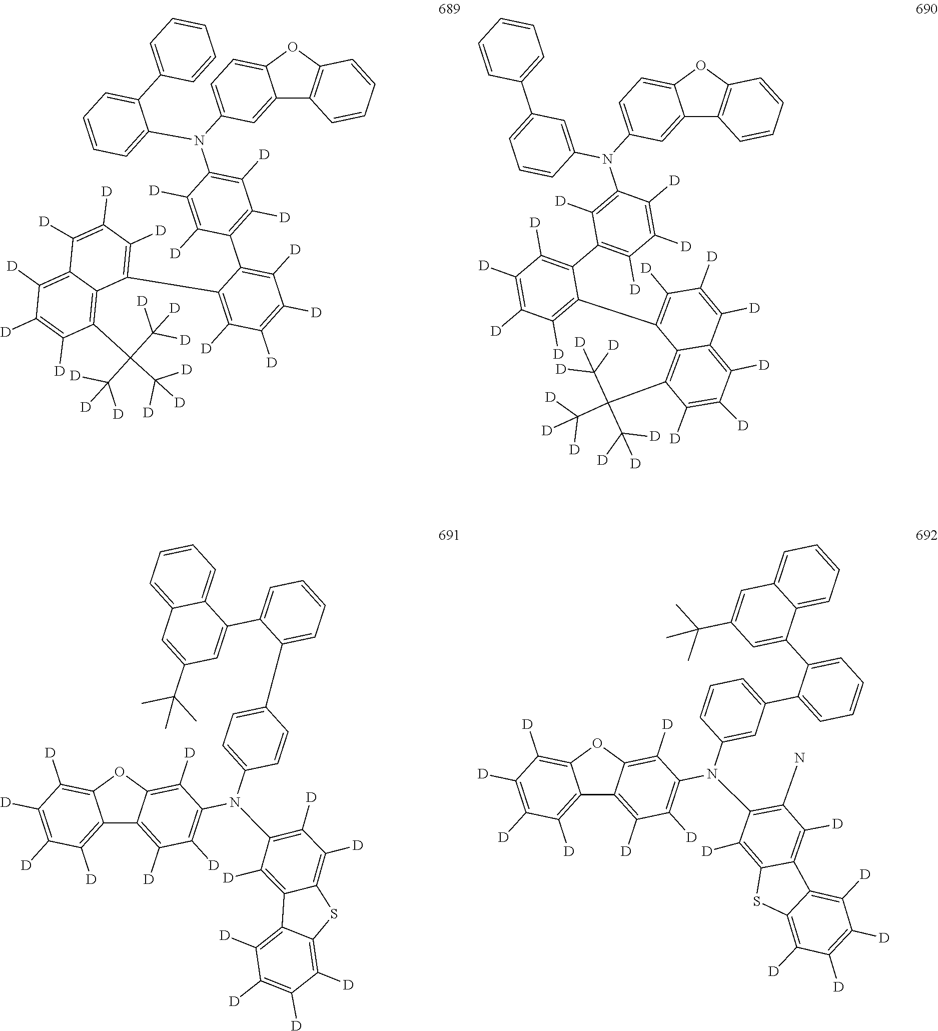

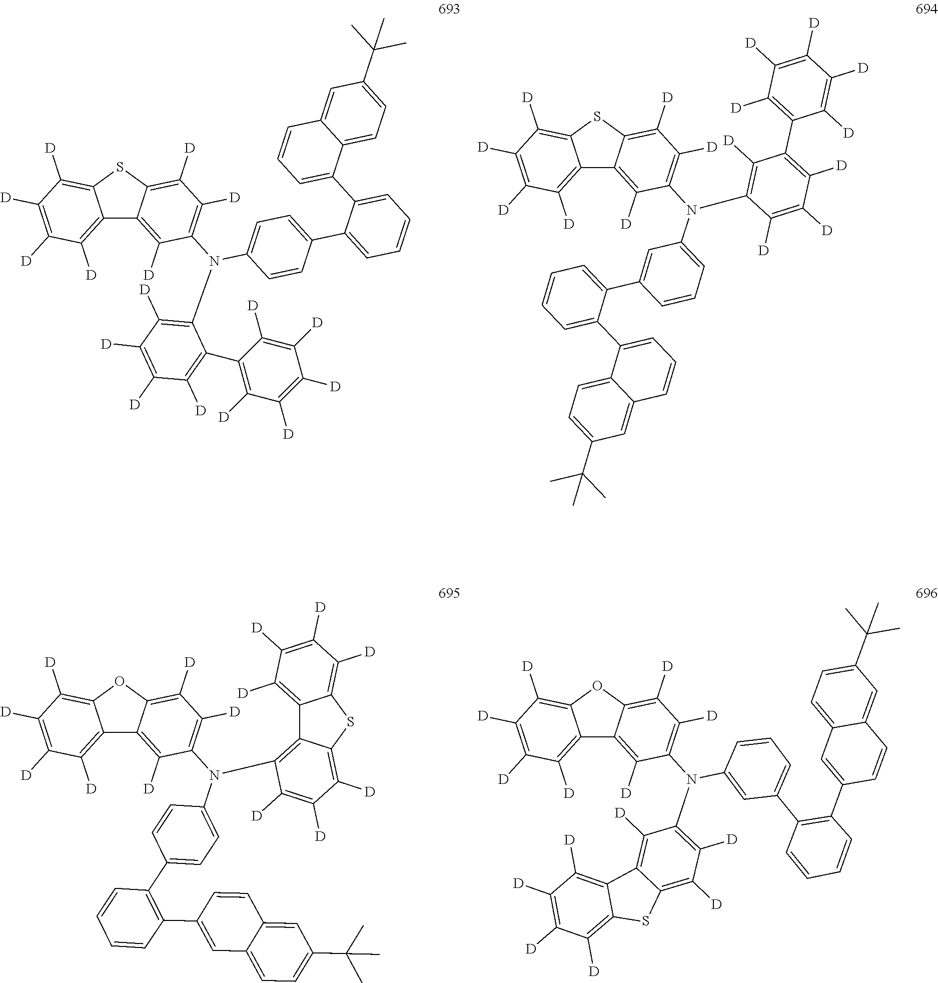

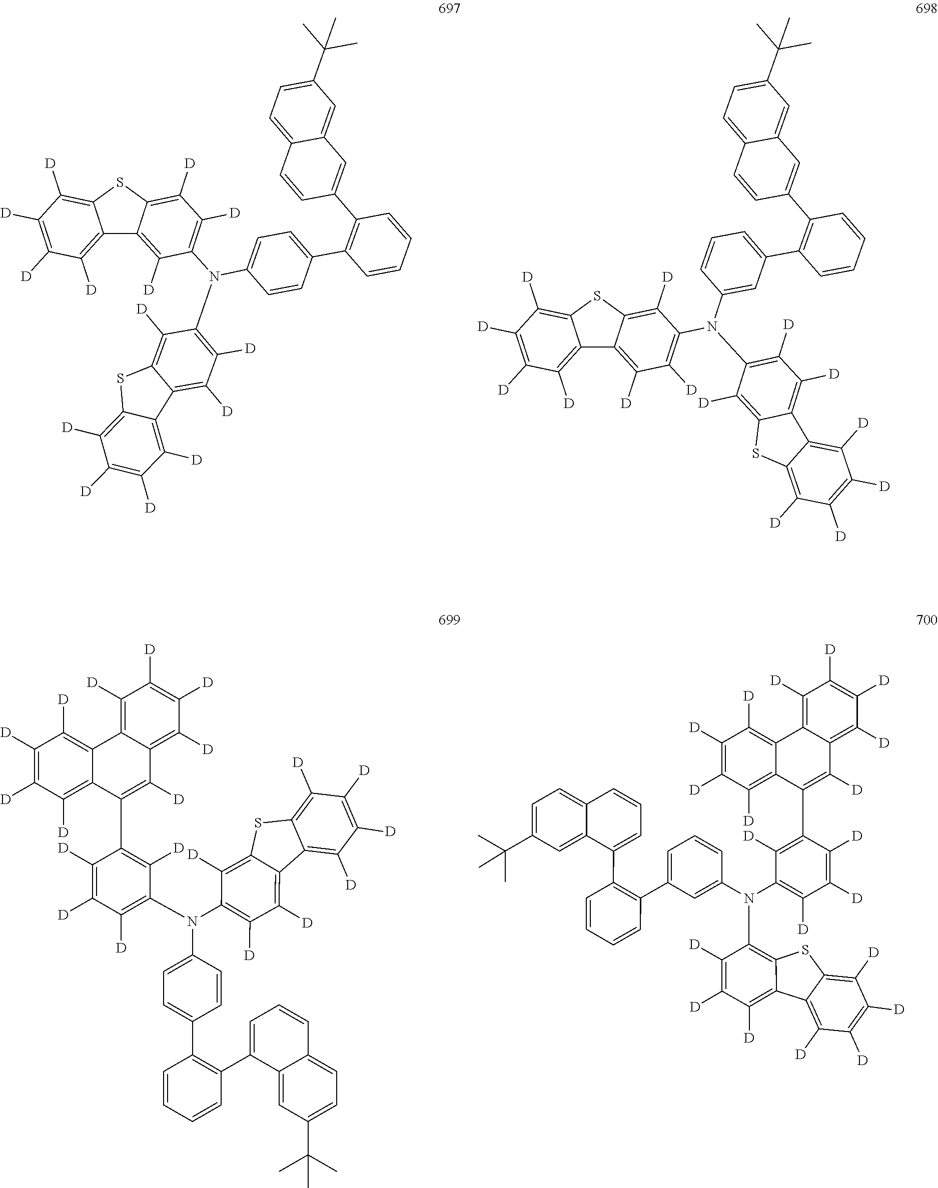

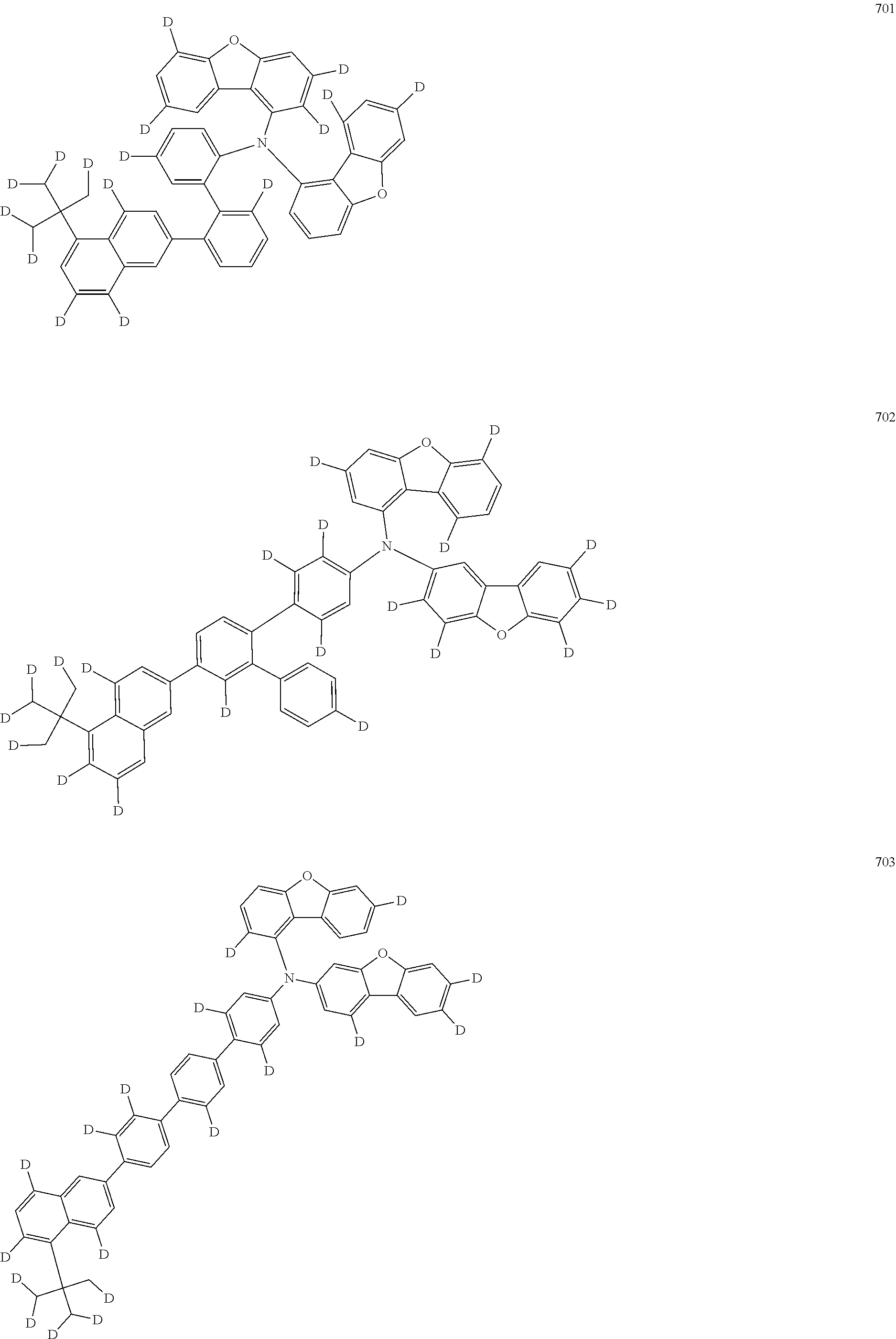

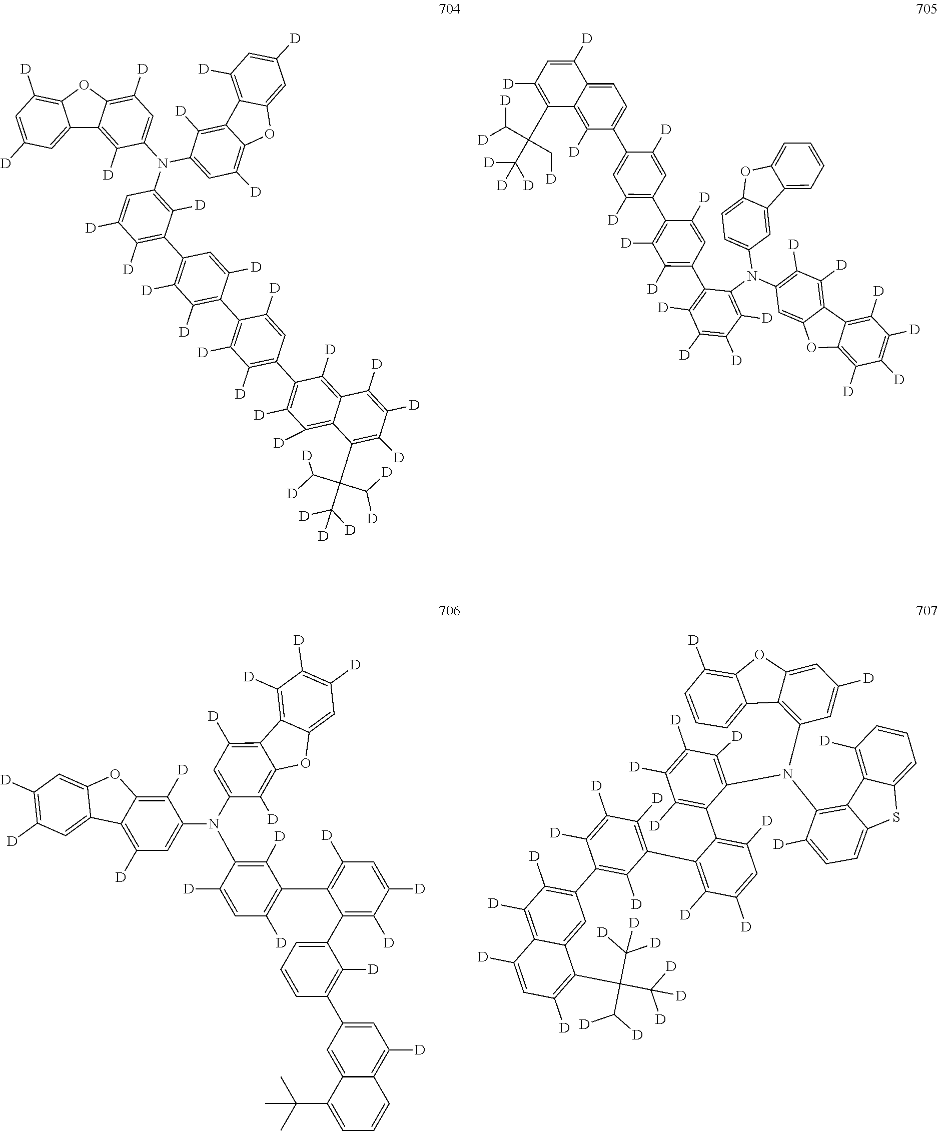

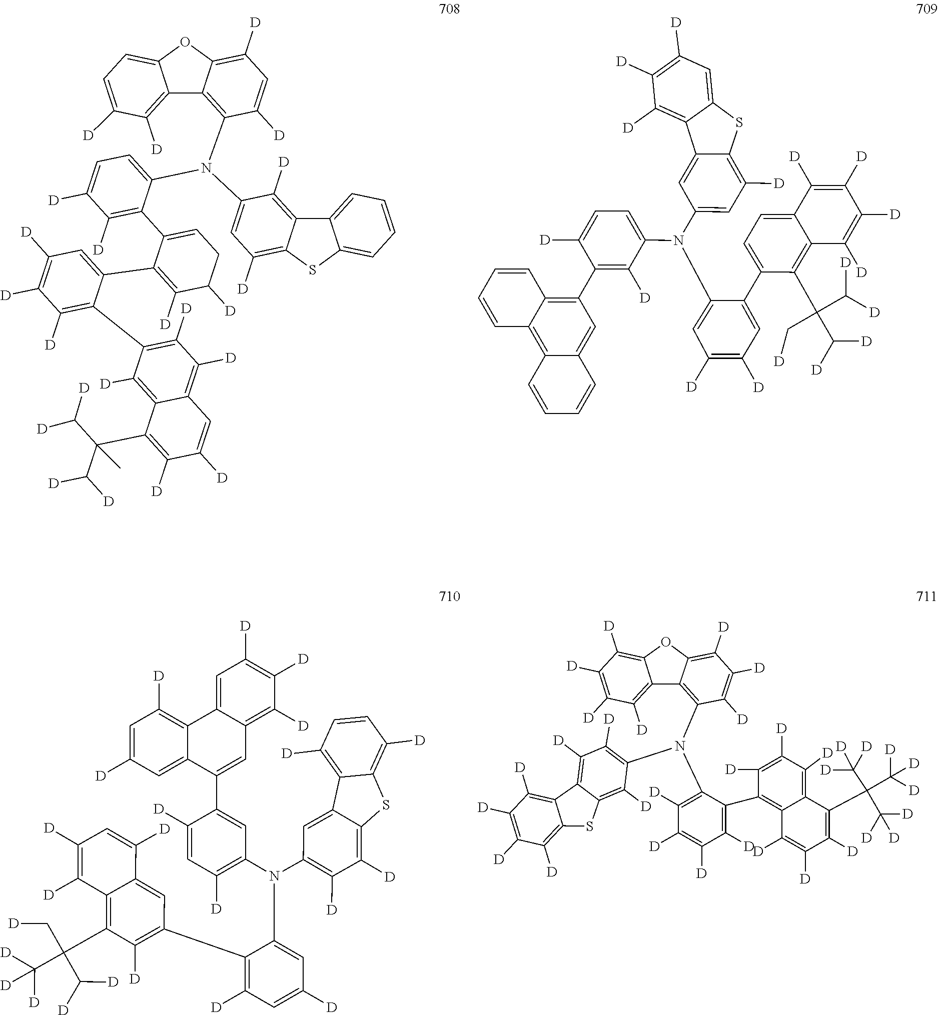

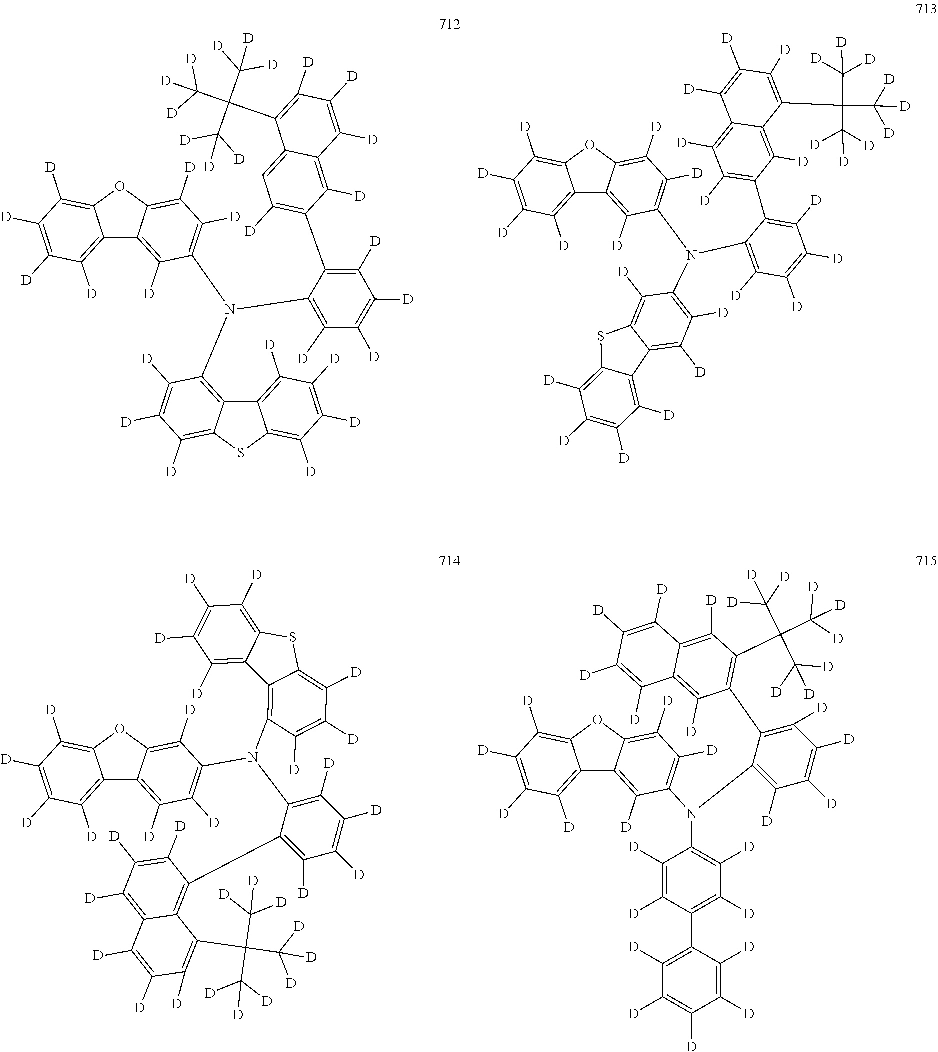

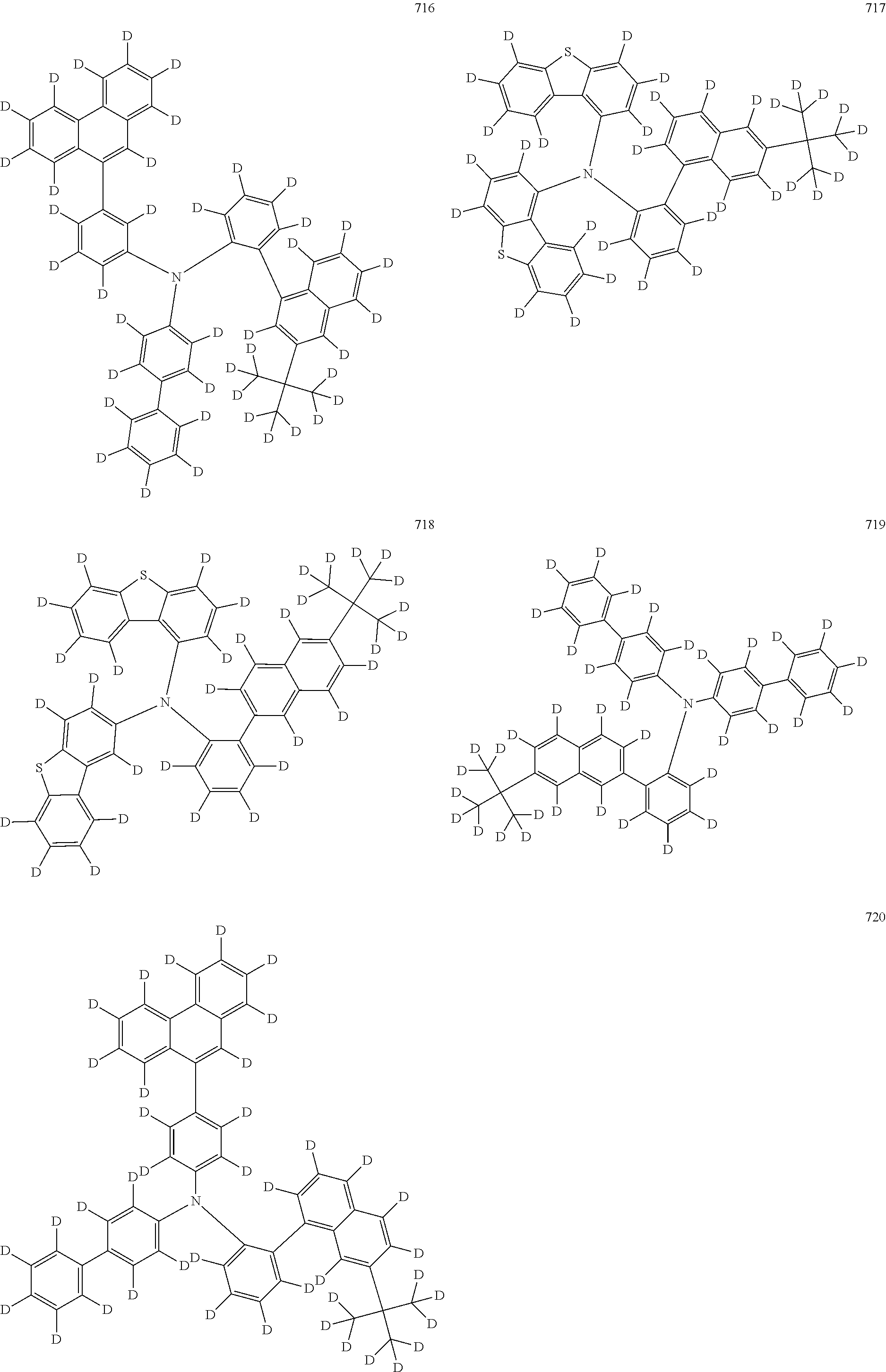

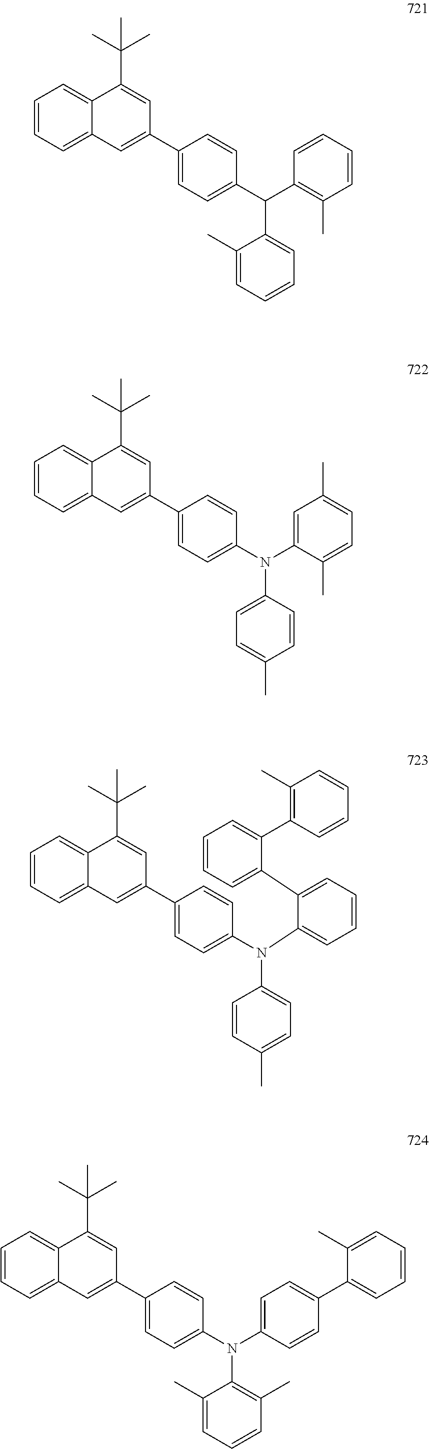

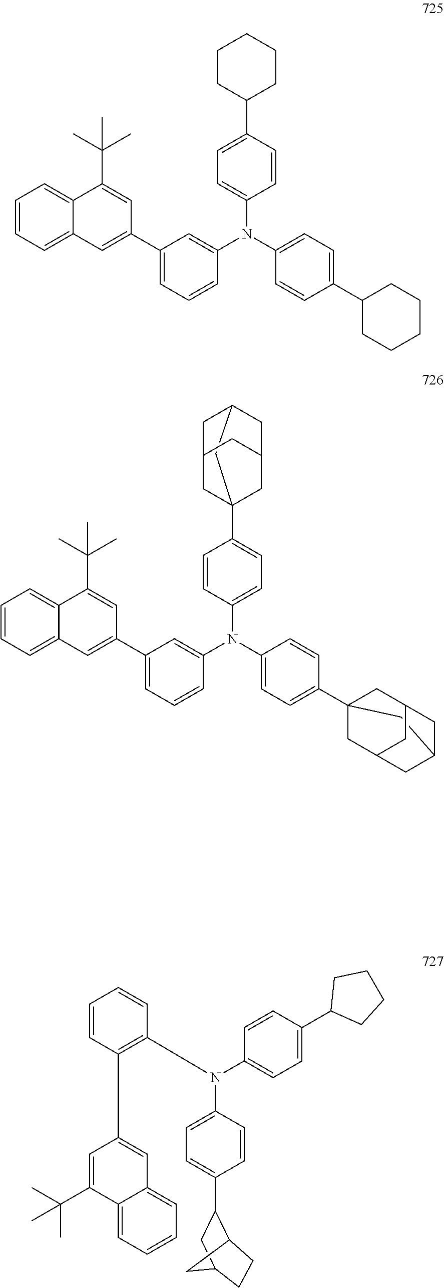 [SMILES: CC(C)(C)c1cc(-c2cccc(N(c3ccc(C4CCCCC4)cc3)c3ccc(C4CCCCC4)cc3)c2)cc2ccccc12 ; CC(C)(C)c1cc(-c2cccc(N(c3ccc(C45CC6CC(CC(C6)C4)C5)cc3)c3ccc(C45CC6CC(CC(C6)C4)C5)cc3)c2)cc2ccccc12 ; CC(C)(C)c1cc(-c2ccccc2N(c2ccc(C3CCCC3)cc2)c2ccc(C3CC4CCC3C4)cc2)cc2ccccc12]

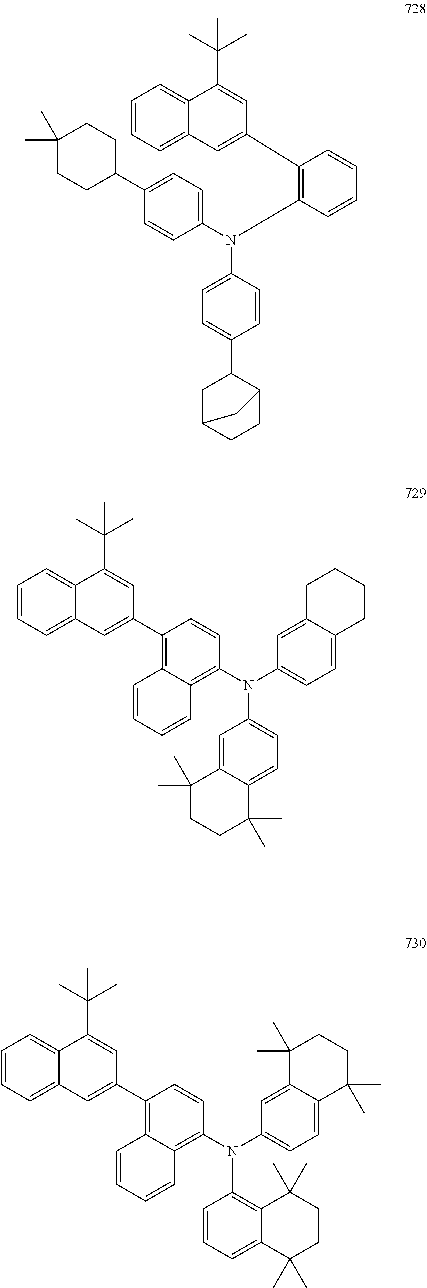 [SMILES: CC1(C)CCC(c2ccc(N(c3ccc(C4CC5CCC4C5)cc3)c3ccccc3-c3cc(C(C)(C)C)c4ccccc4c3)cc2)CC1 ; CC(C)(C)c1cc(-c2ccc(N(c3ccc4c(c3)CCCC4)c3ccc4c(c3)C(C)(C)CCC4(C)C)c3ccccc23)cc2ccccc12 ; CC(C)(C)c1cc(-c2ccc(N(c3ccc4c(c3)C(C)(C)CCC4(C)C)c3cccc4c3C(C)(C)CCC4(C)C)c3ccccc23)cc2ccccc12]

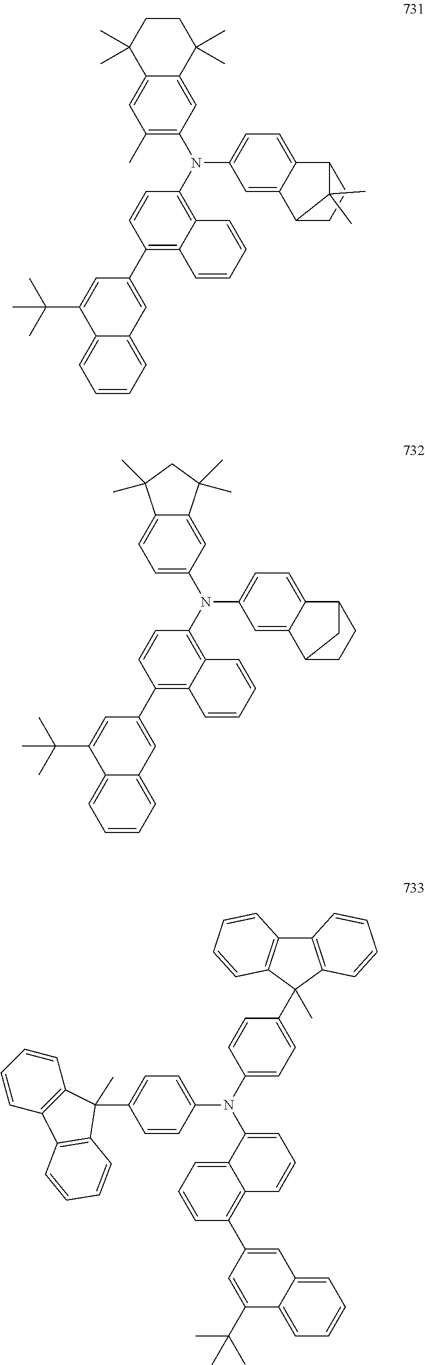

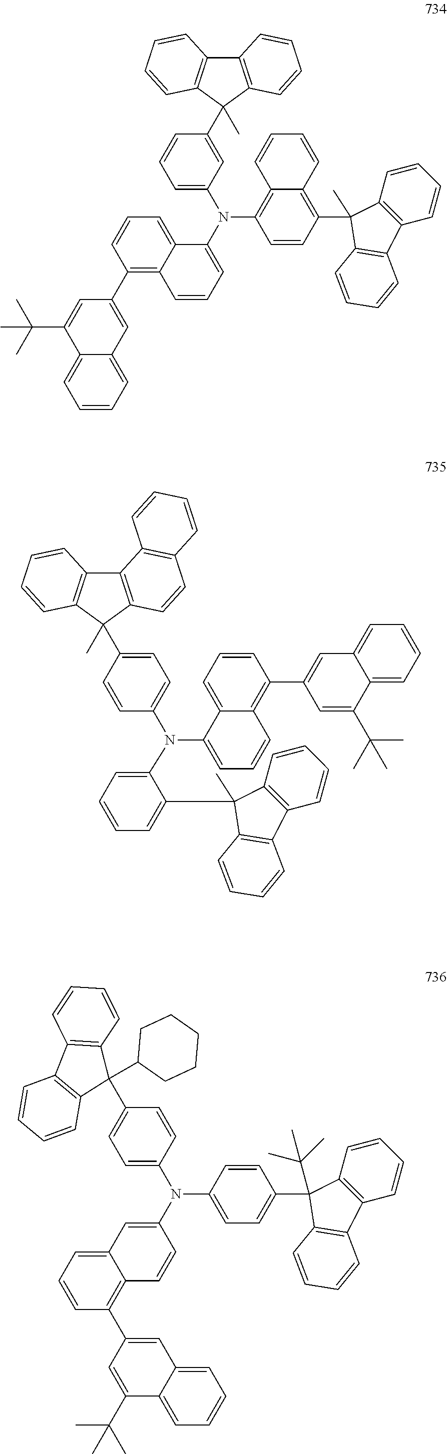 [SMILES: CC(C)(C)c1cc(-c2cccc3c(N(c4cccc(C5(C)c6ccccc6-c6ccccc65)c4)c4ccc(C5(C)c6ccccc6-c6ccccc65)c5ccccc45)cccc23)cc2ccccc12 ; CC(C)(C)c1cc(-c2cccc3c(N(c4ccc(C5(C)c6ccccc6-c6c5ccc5ccccc65)cc4)c4ccccc4C4(C)c5ccccc5-c5ccccc54)cccc23)cc2ccccc12 ; CC(C)(C)c1cc(-c2cccc3cc(N(c4ccc(C5(C6CCCCC6)c6ccccc6-c6ccccc65)cc4)c4ccc(C5(C(C)(C)C)c6ccccc6-c6ccccc65)cc4)ccc23)cc2ccccc12]

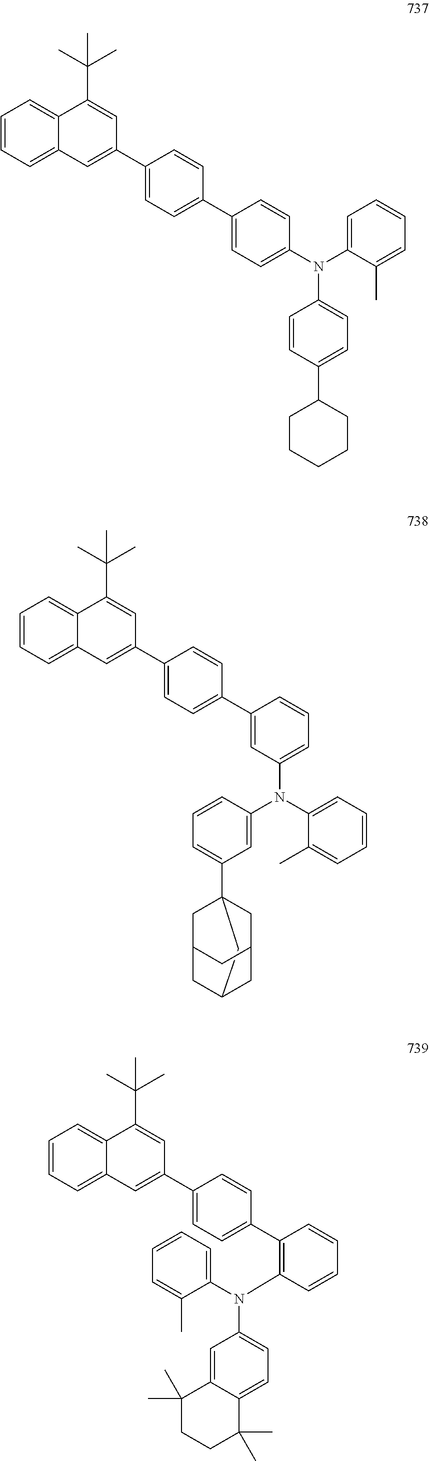 [SMILES: Cc1ccccc1N(c1ccc(-c2ccc(-c3cc(C(C)(C)C)c4ccccc4c3)cc2)cc1)c1ccc(C2CCCCC2)cc1 ; Cc1ccccc1N(c1cccc(-c2ccc(-c3cc(C(C)(C)C)c4ccccc4c3)cc2)c1)c1cccc(C23CC4CC(CC(C4)C2)C3)c1 ; Cc1ccccc1N(c1ccc2c(c1)C(C)(C)CCC2(C)C)c1ccccc1-c1ccc(-c2cc(C(C)(C)C)c3ccccc3c2)cc1]

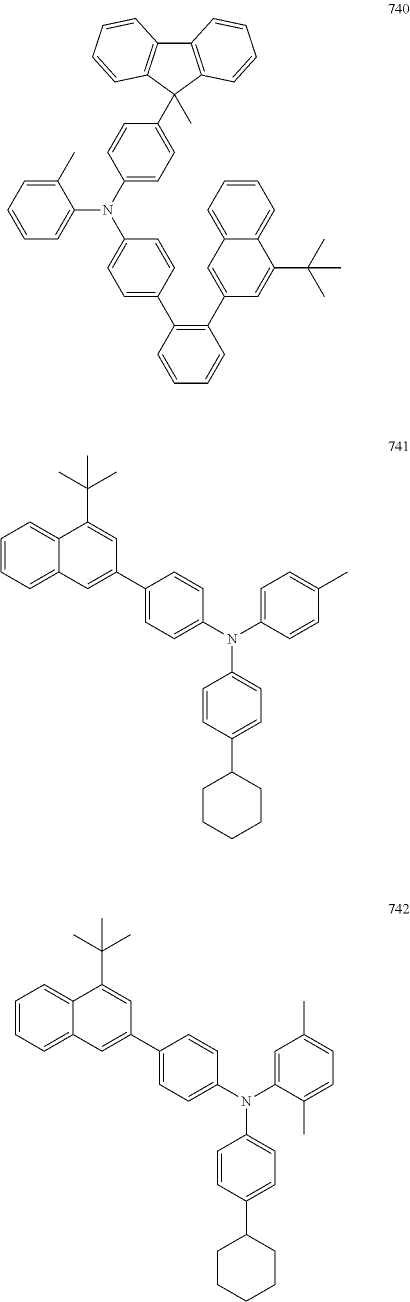 [SMILES: Cc1ccccc1N(c1ccc(-c2ccccc2-c2cc(C(C)(C)C)c3ccccc3c2)cc1)c1ccc(C2(C)c3ccccc3-c3ccccc32)cc1 ; Cc1ccc(N(c2ccc(-c3cc(C(C)(C)C)c4ccccc4c3)cc2)c2ccc(C3CCCCC3)cc2)cc1 ; Cc1ccc(C)c(N(c2ccc(-c3cc(C(C)(C)C)c4ccccc4c3)cc2)c2ccc(C3CCCCC3)cc2)c1]

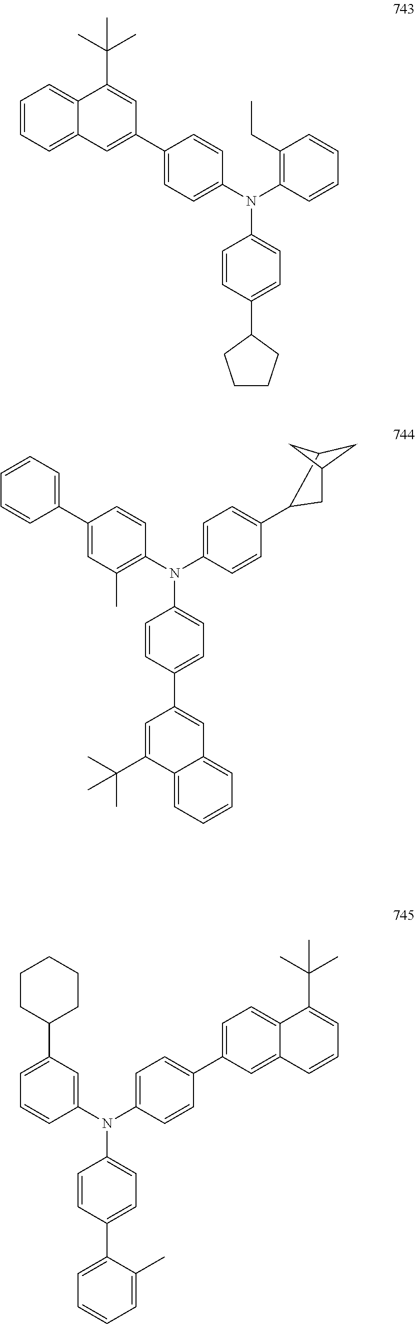 [SMILES: CCc1ccccc1N(c1ccc(-c2cc(C(C)(C)C)c3ccccc3c2)cc1)c1ccc(C2CCCC2)cc1 ; Cc1cc(-c2ccccc2)ccc1N(c1ccc(-c2cc(C(C)(C)C)c3ccccc3c2)cc1)c1ccc(C2CC3CC2C3)cc1 ; Cc1ccccc1-c1ccc(N(c2ccc(-c3ccc4c(C(C)(C)C)cccc4c3)cc2)c2cccc(C3CCCCC3)c2)cc1]

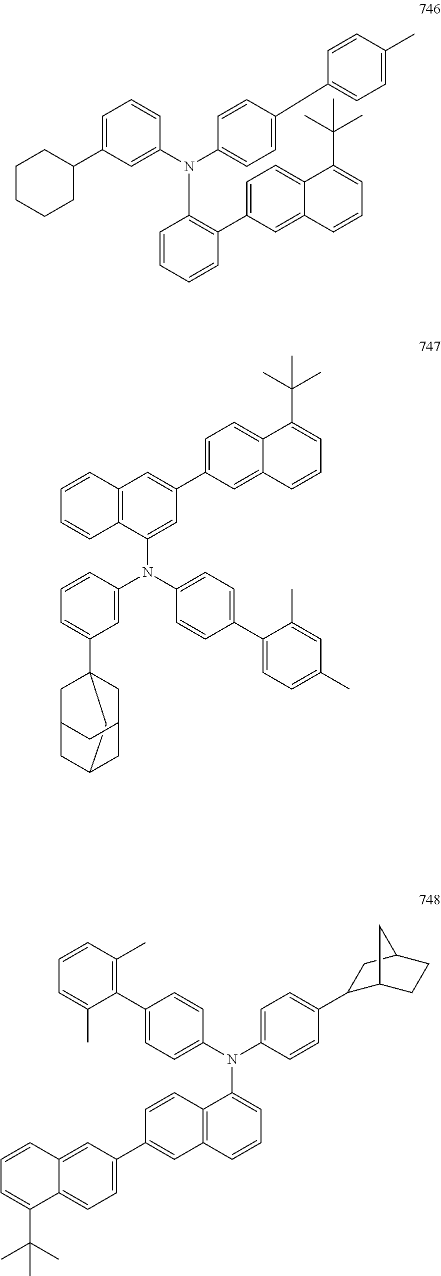 [SMILES: Cc1ccc(-c2ccc(N(c3cccc(C4CCCCC4)c3)c3ccccc3-c3ccc4c(C(C)(C)C)cccc4c3)cc2)cc1 ; Cc1ccc(-c2ccc(N(c3cccc(C45CC6CC(CC(C6)C4)C5)c3)c3cc(-c4ccc5c(C(C)(C)C)cccc5c4)cc4ccccc34)cc2)c(C)c1 ; Cc1cccc(C)c1-c1ccc(N(c2ccc(C3CC4CCC3C4)cc2)c2cccc3cc(-c4ccc5c(C(C)(C)C)cccc5c4)ccc23)cc1]

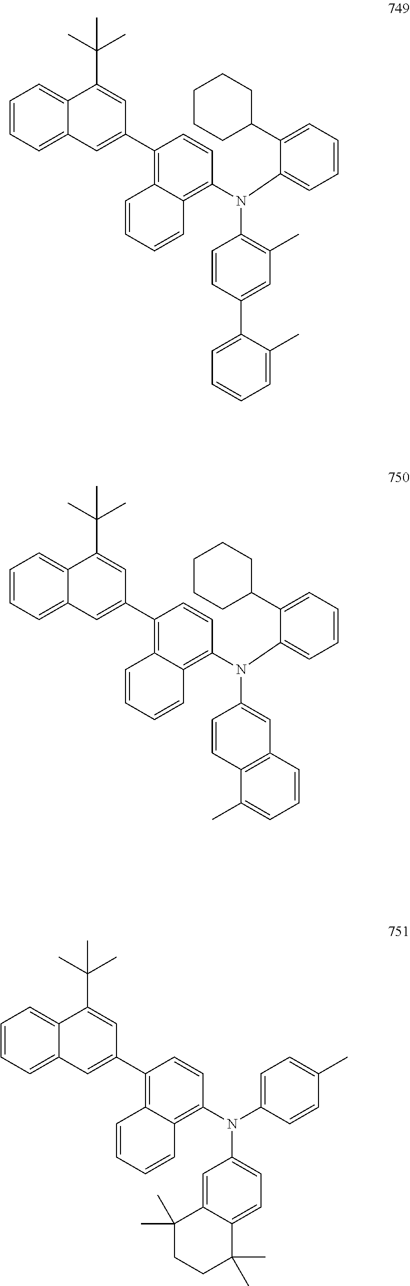 [SMILES: Cc1ccccc1-c1ccc(N(c2ccccc2C2CCCCC2)c2ccc(-c3cc(C(C)(C)C)c4ccccc4c3)c3ccccc23)c(C)c1 ; Cc1cccc2cc(N(c3ccccc3C3CCCCC3)c3ccc(-c4cc(C(C)(C)C)c5ccccc5c4)c4ccccc34)ccc12 ; Cc1ccc(N(c2ccc3c(c2)C(C)(C)CCC3(C)C)c2ccc(-c3cc(C(C)(C)C)c4ccccc4c3)c3ccccc23)cc1]

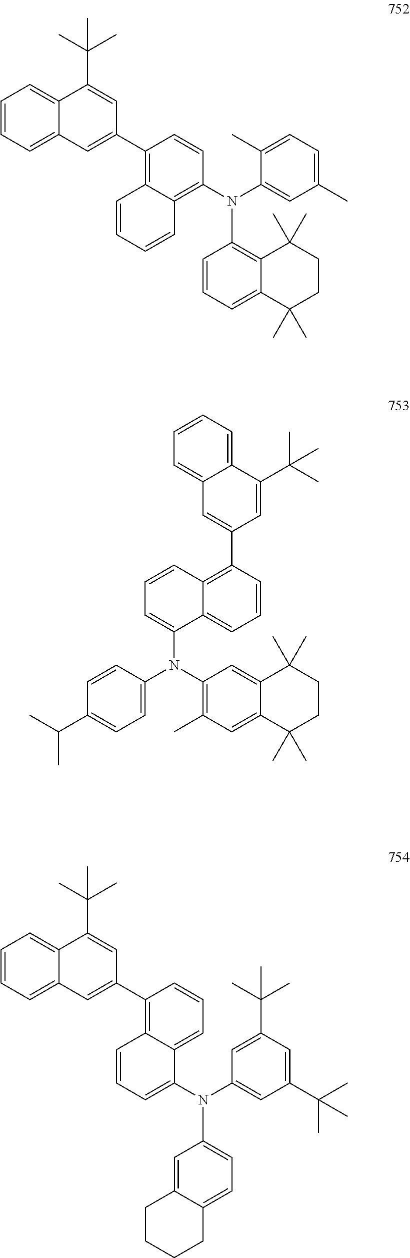

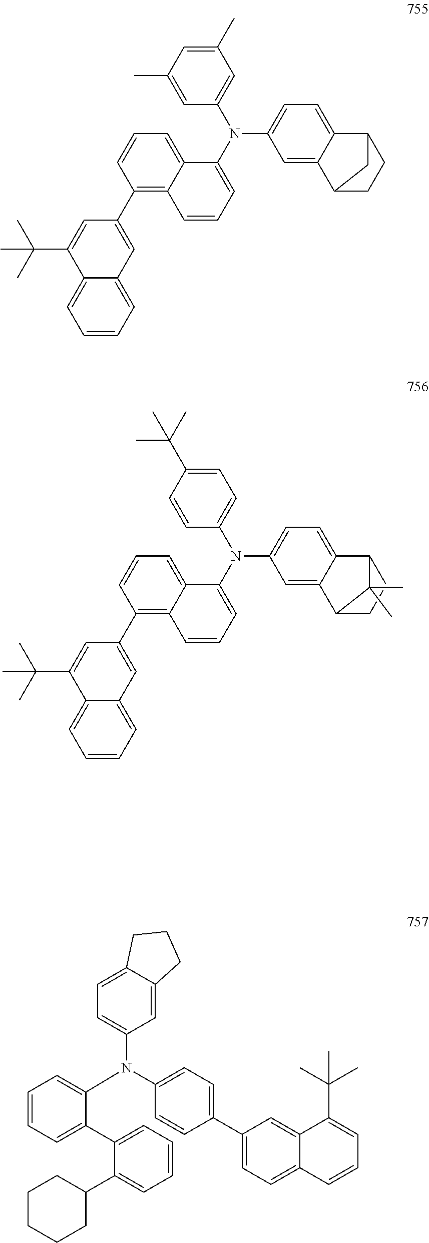 [SMILES: Cc1cc(C)cc(N(c2ccc3c(c2)C2CCC3C2)c2cccc3c(-c4cc(C(C)(C)C)c5ccccc5c4)cccc23)c1 ; CC(C)(C)c1ccc(N(c2ccc3c(c2)C2CCC3C2(C)C)c2cccc3c(-c4cc(C(C)(C)C)c5ccccc5c4)cccc23)cc1 ; CC(C)(C)c1cccc2ccc(-c3ccc(N(c4ccc5c(c4)CCC5)c4ccccc4-c4ccccc4C4CCCCC4)cc3)cc12]

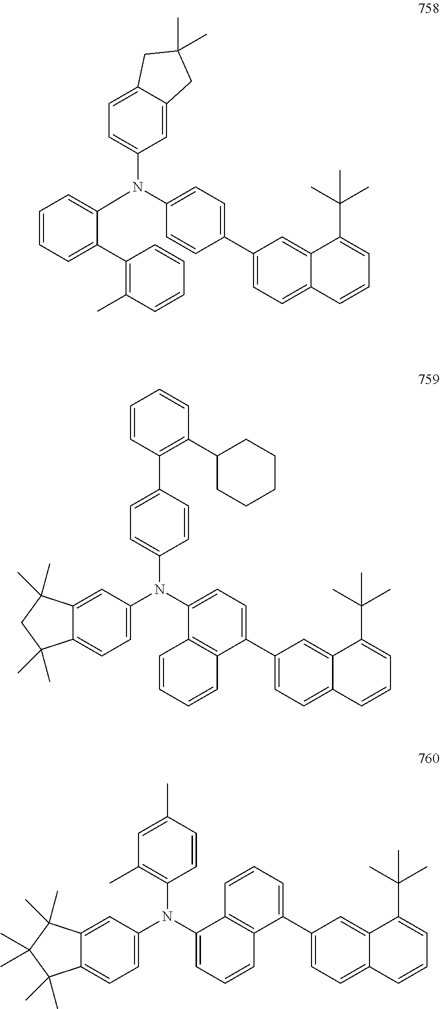

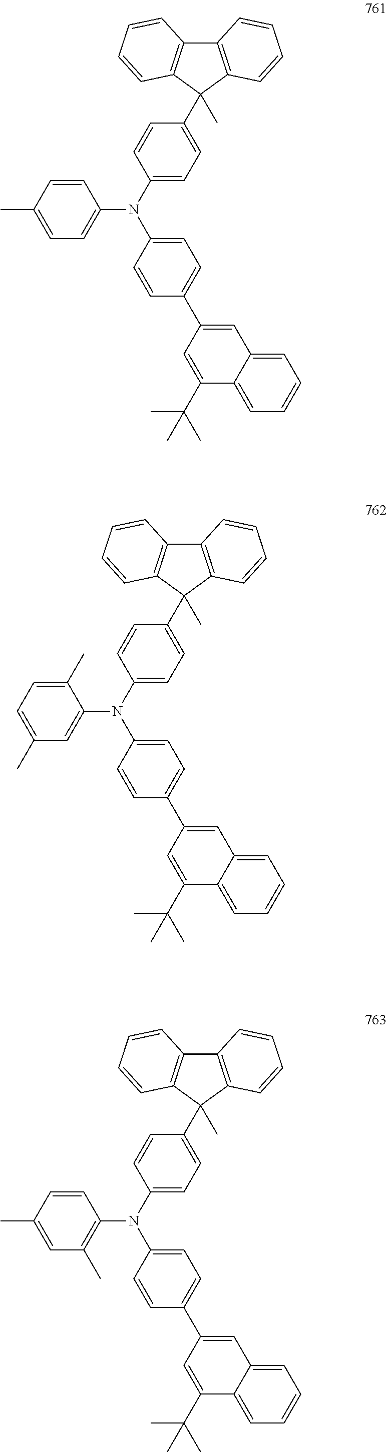 [SMILES: Cc1ccc(N(c2ccc(-c3cc(C(C)(C)C)c4ccccc4c3)cc2)c2ccc(C3(C)c4ccccc4-c4ccccc43)cc2)cc1 ; Cc1ccc(C)c(N(c2ccc(-c3cc(C(C)(C)C)c4ccccc4c3)cc2)c2ccc(C3(C)c4ccccc4-c4ccccc43)cc2)c1 ; Cc1ccc(N(c2ccc(-c3cc(C(C)(C)C)c4ccccc4c3)cc2)c2ccc(C3(C)c4ccccc4-c4ccccc43)cc2)c(C)c1]

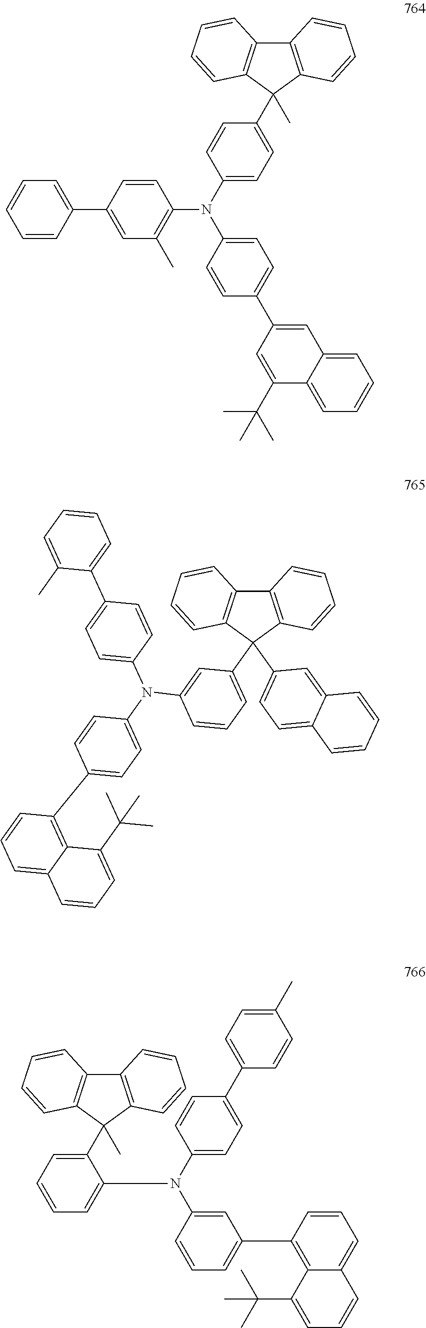 [SMILES: Cc1cc(-c2ccccc2)ccc1N(c1ccc(-c2cc(C(C)(C)C)c3ccccc3c2)cc1)c1ccc(C2(C)c3ccccc3-c3ccccc32)cc1 ; Cc1ccccc1-c1ccc(N(c2ccc(-c3cccc4cccc(C(C)(C)C)c34)cc2)c2cccc(C3(c4ccc5ccccc5c4)c4ccccc4-c4ccccc43)c2)cc1 ; Cc1ccc(-c2ccc(N(c3cccc(-c4cccc5cccc(C(C)(C)C)c45)c3)c3ccccc3C3(C)c4ccccc4-c4ccccc43)cc2)cc1]

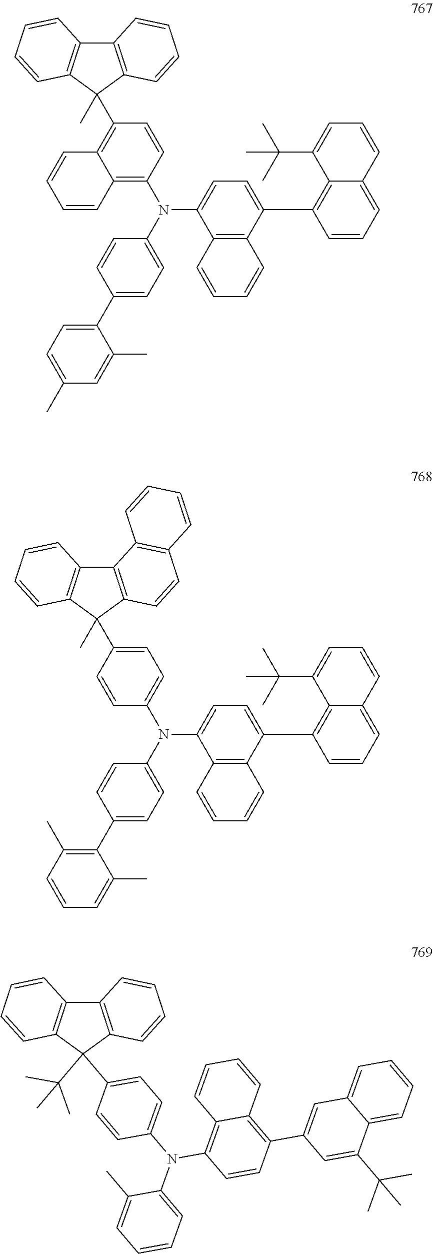 [SMILES: Cc1ccc(-c2ccc(N(c3ccc(-c4cccc5cccc(C(C)(C)C)c45)c4ccccc34)c3ccc(C4(C)c5ccccc5-c5ccccc54)c4ccccc34)cc2)c(C)c1 ; Cc1cccc(C)c1-c1ccc(N(c2ccc(C3(C)c4ccccc4-c4c3ccc3ccccc43)cc2)c2ccc(-c3cccc4cccc(C(C)(C)C)c34)c3ccccc23)cc1 ; Cc1ccccc1N(c1ccc(C2(C(C)(C)C)c3ccccc3-c3ccccc32)cc1)c1ccc(-c2cc(C(C)(C)C)c3ccccc3c2)c2ccccc12]

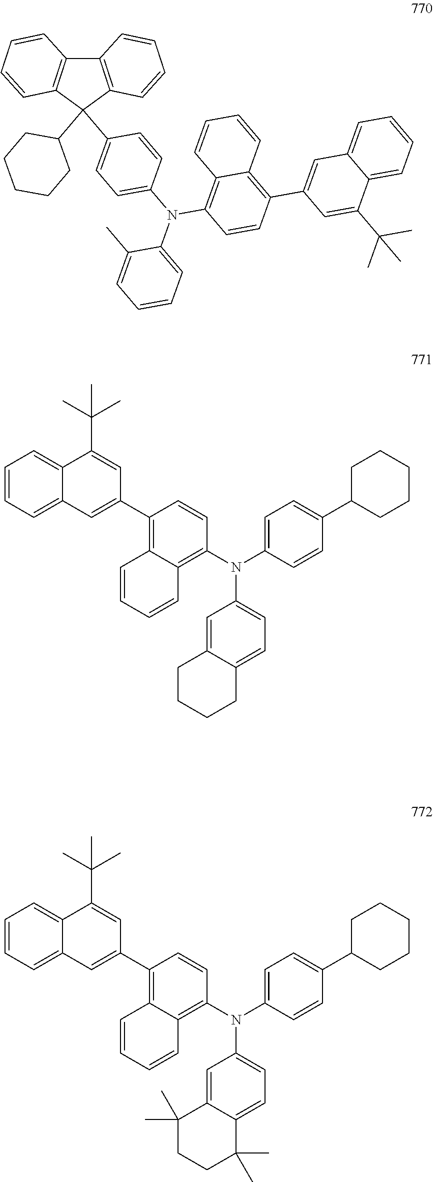 [SMILES: Cc1ccccc1N(c1ccc(C2(C3CCCCC3)c3ccccc3-c3ccccc32)cc1)c1ccc(-c2cc(C(C)(C)C)c3ccccc3c2)c2ccccc12 ; CC(C)(C)c1cc(-c2ccc(N(c3ccc(C4CCCCC4)cc3)c3ccc4c(c3)CCCC4)c3ccccc23)cc2ccccc12 ; CC(C)(C)c1cc(-c2ccc(N(c3ccc(C4CCCCC4)cc3)c3ccc4c(c3)C(C)(C)CCC4(C)C)c3ccccc23)cc2ccccc12]

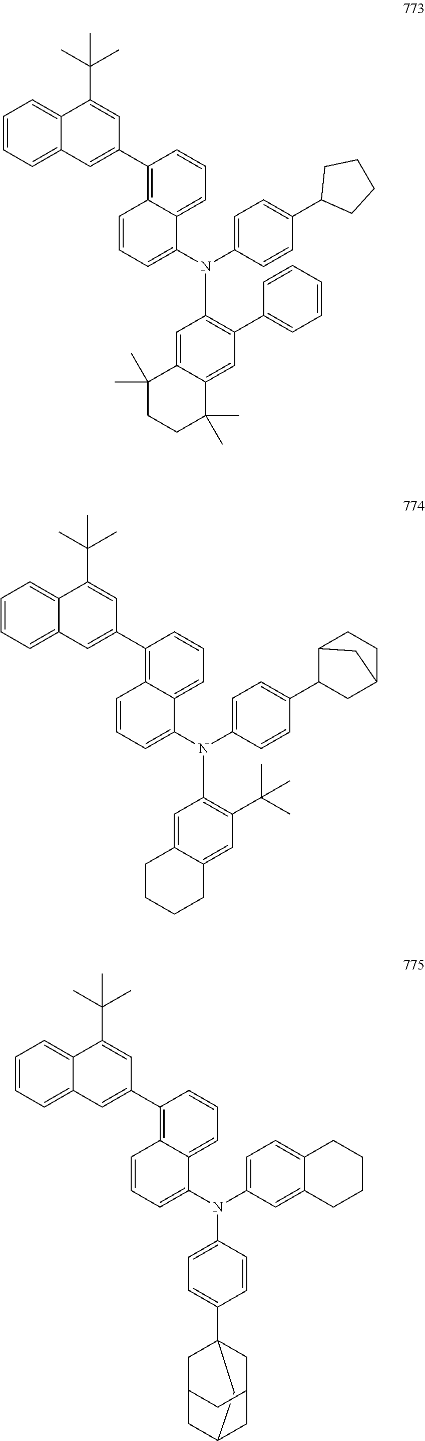 [SMILES: CC(C)(C)c1cc(-c2cccc3c(N(c4ccc(C5CCCC5)cc4)c4cc5c(cc4-c4ccccc4)C(C)(C)CCC5(C)C)cccc23)cc2ccccc12 ; CC(C)(C)c1cc2c(cc1N(c1ccc(C3CC4CCC3C4)cc1)c1cccc3c(-c4cc(C(C)(C)C)c5ccccc5c4)cccc13)CCCC2 ; CC(C)(C)c1cc(-c2cccc3c(N(c4ccc(C56CC7CC(CC(C7)C5)C6)cc4)c4ccc5c(c4)CCCC5)cccc23)cc2ccccc12]

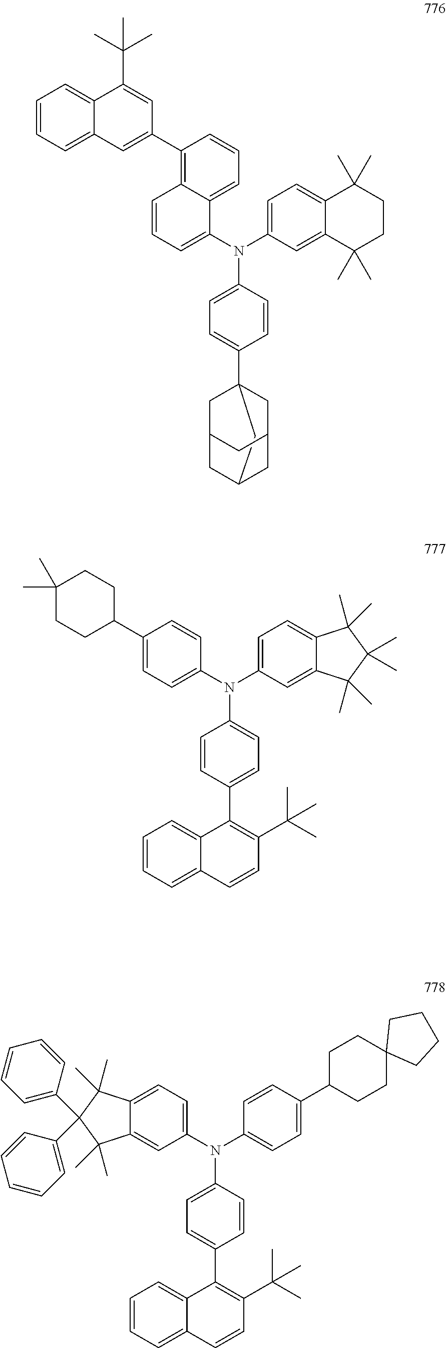

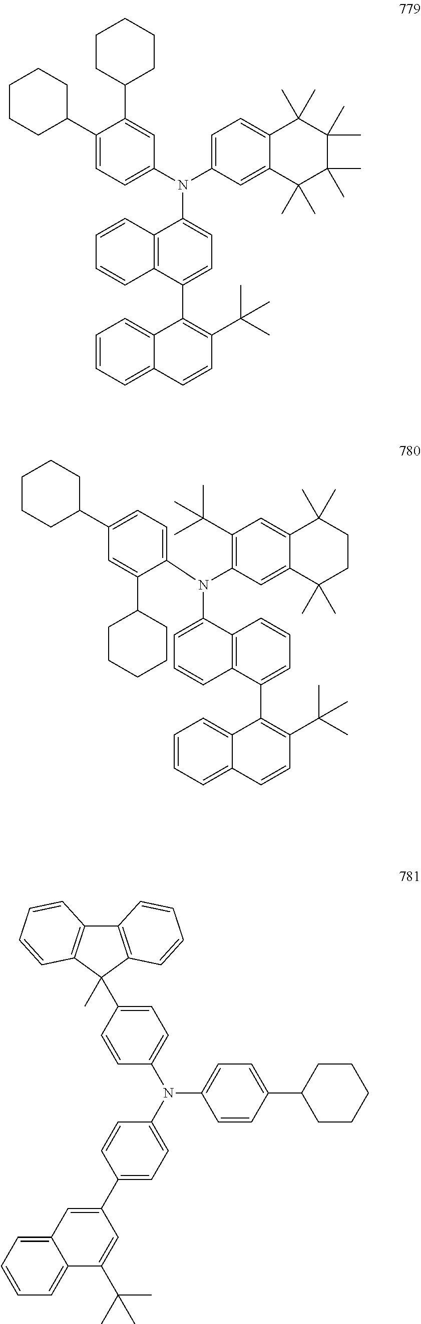 [SMILES: CC(C)(C)c1ccc2ccccc2c1-c1ccc(N(c2ccc(C3CCCCC3)c(C3CCCCC3)c2)c2ccc3c(c2)C(C)(C)C(C)(C)C(C)(C)C3(C)C)c2ccccc12 ; CC(C)(C)c1cc2c(cc1N(c1ccc(C3CCCCC3)cc1C1CCCCC1)c1cccc3c(-c4c(C(C)(C)C)ccc5ccccc45)cccc13)C(C)(C)CCC2(C)C ; CC(C)(C)c1cc(-c2ccc(N(c3ccc(C4CCCCC4)cc3)c3ccc(C4(C)c5ccccc5-c5ccccc54)cc3)cc2)cc2ccccc12]

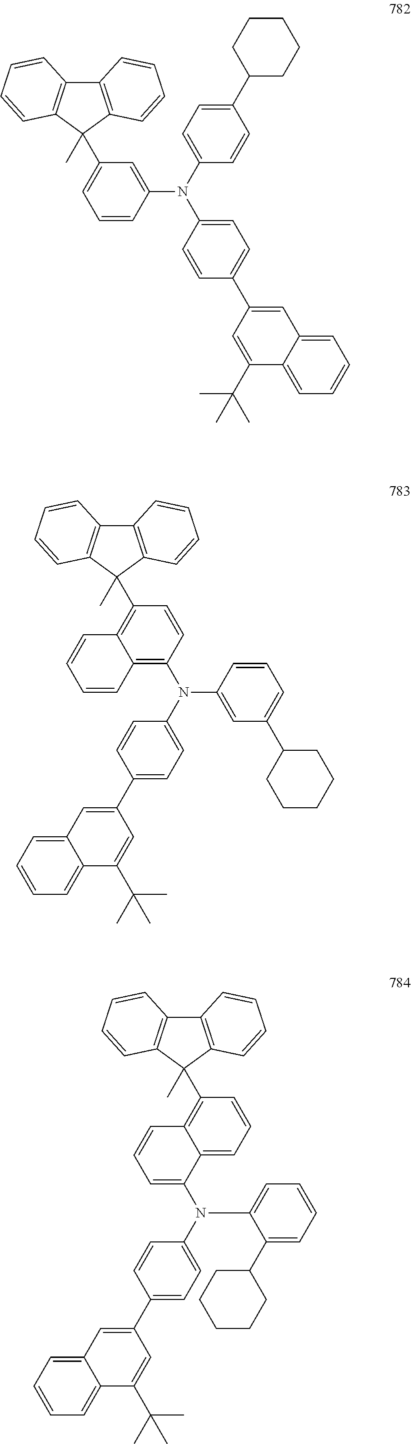 [SMILES: CC(C)(C)c1cc(-c2ccc(N(c3ccc(C4CCCCC4)cc3)c3cccc(C4(C)c5ccccc5-c5ccccc54)c3)cc2)cc2ccccc12 ; CC(C)(C)c1cc(-c2ccc(N(c3cccc(C4CCCCC4)c3)c3ccc(C4(C)c5ccccc5-c5ccccc54)c4ccccc34)cc2)cc2ccccc12 ; CC(C)(C)c1cc(-c2ccc(N(c3ccccc3C3CCCCC3)c3cccc4c(C5(C)c6ccccc6-c6ccccc65)cccc34)cc2)cc2ccccc12]

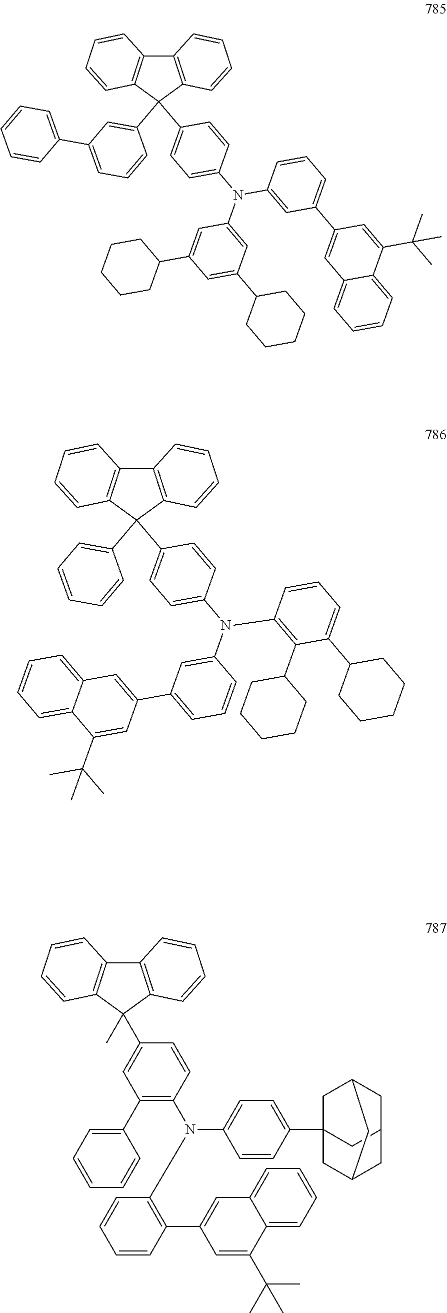 [SMILES: CC(C)(C)c1cc(-c2cccc(N(c3ccc(C4(c5cccc(-c6ccccc6)c5)c5ccccc5-c5ccccc54)cc3)c3cc(C4CCCCC4)cc(C4CCCCC4)c3)c2)cc2ccccc12 ; CC(C)(C)c1cc(-c2cccc(N(c3ccc(C4(c5ccccc5)c5ccccc5-c5ccccc54)cc3)c3cccc(C4CCCCC4)c3C3CCCCC3)c2)cc2ccccc12 ; CC(C)(C)c1cc(-c2ccccc2N(c2ccc(C34CC5CC(CC(C5)C3)C4)cc2)c2ccc(C3(C)c4ccccc4-c4ccccc43)cc2-c2ccccc2)cc2ccccc12]

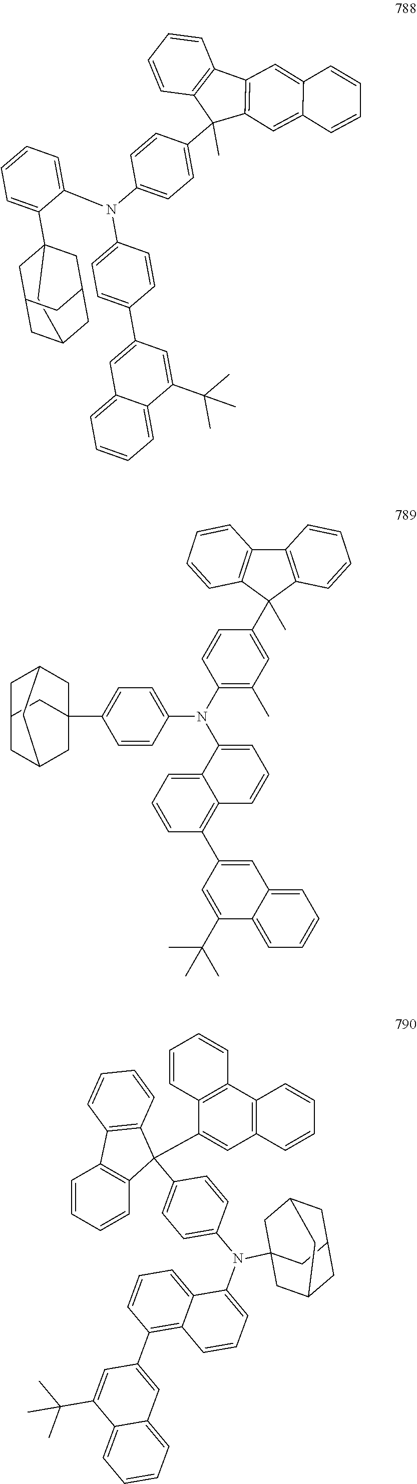 [SMILES: CC(C)(C)c1cc(-c2ccc(N(c3ccc(C4(C)c5ccccc5-c5cc6ccccc6cc54)cc3)c3ccccc3C34CC5CC(CC(C5)C3)C4)cc2)cc2ccccc12 ; Cc1cc(C2(C)c3ccccc3-c3ccccc32)ccc1N(c1ccc(C23CC4CC(CC(C4)C2)C3)cc1)c1cccc2c(-c3cc(C(C)(C)C)c4ccccc4c3)cccc12 ; CC(C)(C)c1cc(-c2cccc3c(N(c4ccc(C5(c6cc7ccccc7c7ccccc67)c6ccccc6-c6ccccc65)cc4)C45CC6CC(CC(C6)C4)C5)cccc23)cc2ccccc12]

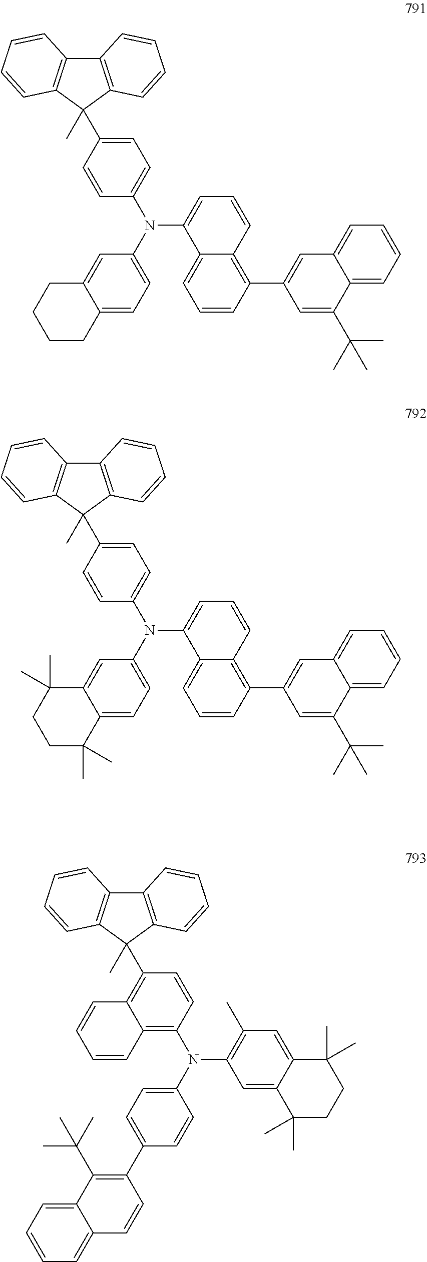 [SMILES: CC(C)(C)c1cc(-c2cccc3c(N(c4ccc(C5(C)c6ccccc6-c6ccccc65)cc4)c4ccc5c(c4)CCCC5)cccc23)cc2ccccc12 ; CC(C)(C)c1cc(-c2cccc3c(N(c4ccc(C5(C)c6ccccc6-c6ccccc65)cc4)c4ccc5c(c4)C(C)(C)CCC5(C)C)cccc23)cc2ccccc12 ; Cc1cc2c(cc1N(c1ccc(-c3ccc4ccccc4c3C(C)(C)C)cc1)c1ccc(C3(C)c4ccccc4-c4ccccc43)c3ccccc13)C(C)(C)CCC2(C)C]

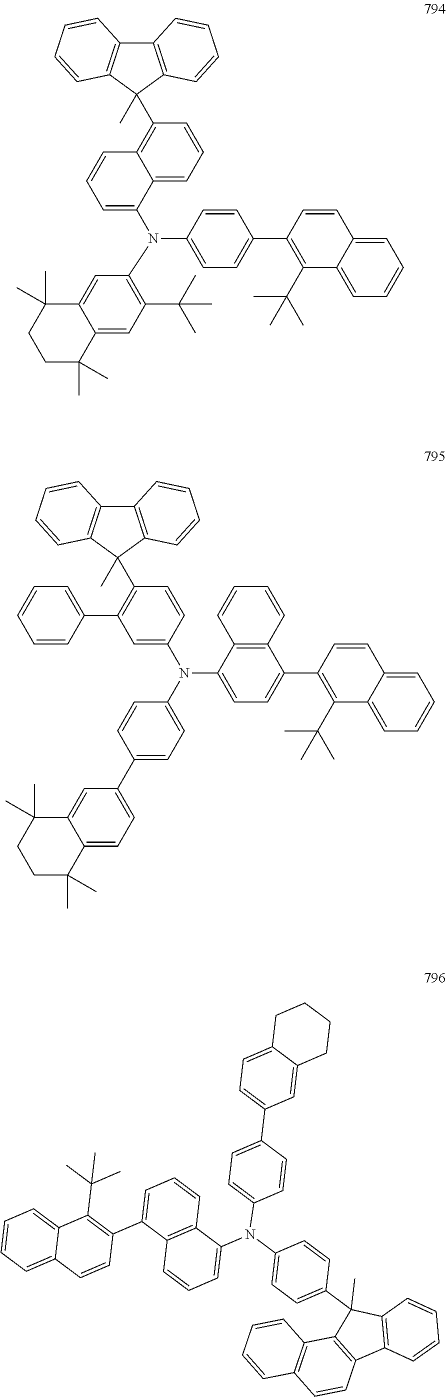 [SMILES: CC(C)(C)c1cc2c(cc1N(c1ccc(-c3ccc4ccccc4c3C(C)(C)C)cc1)c1cccc3c(C4(C)c5ccccc5-c5ccccc54)cccc13)C(C)(C)CCC2(C)C ; CC(C)(C)c1c(-c2ccc(N(c3ccc(-c4ccc5c(c4)C(C)(C)CCC5(C)C)cc3)c3ccc(C4(C)c5ccccc5-c5ccccc54)c(-c4ccccc4)c3)c3ccccc23)ccc2ccccc12 ; CC(C)(C)c1c(-c2cccc3c(N(c4ccc(-c5ccc6c(c5)CCCC6)cc4)c4ccc(C5(C)c6ccccc6-c6ccc7ccccc7c65)cc4)cccc23)ccc2ccccc12]

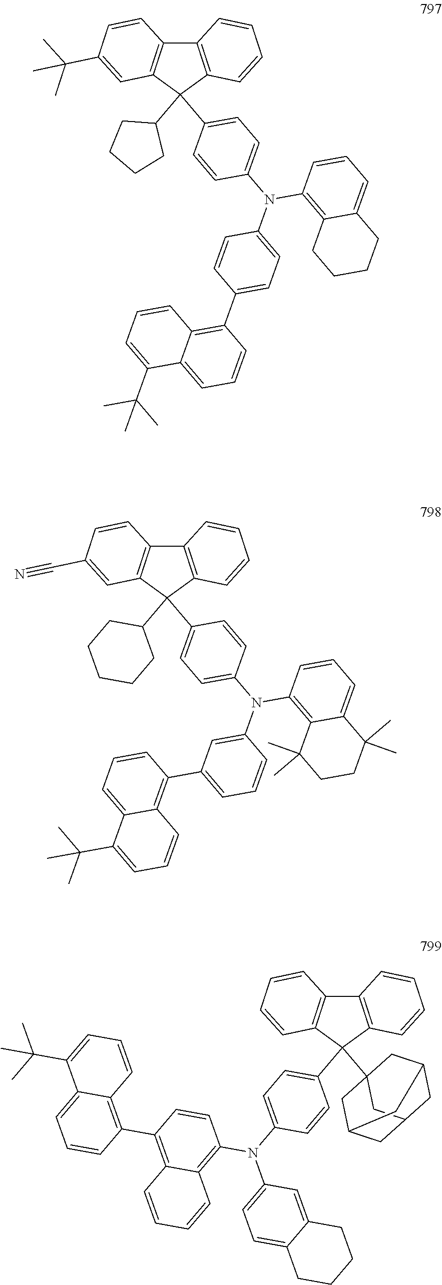

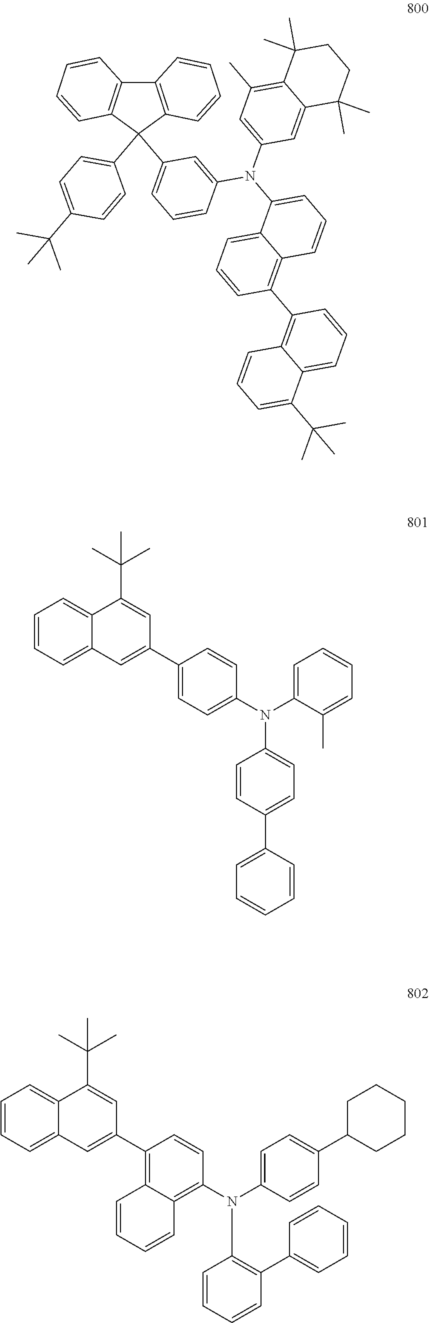 [SMILES: Cc1cc(N(c2cccc(C3(c4ccc(C(C)(C)C)cc4)c4ccccc4-c4ccccc43)c2)c2cccc3c(-c4cccc5c(C(C)(C)C)cccc45)cccc23)cc2c1C(C)(C)CCC2(C)C ; Cc1ccccc1N(c1ccc(-c2ccccc2)cc1)c1ccc(-c2cc(C(C)(C)C)c3ccccc3c2)cc1 ; CC(C)(C)c1cc(-c2ccc(N(c3ccc(C4CCCCC4)cc3)c3ccccc3-c3ccccc3)c3ccccc23)cc2ccccc12]

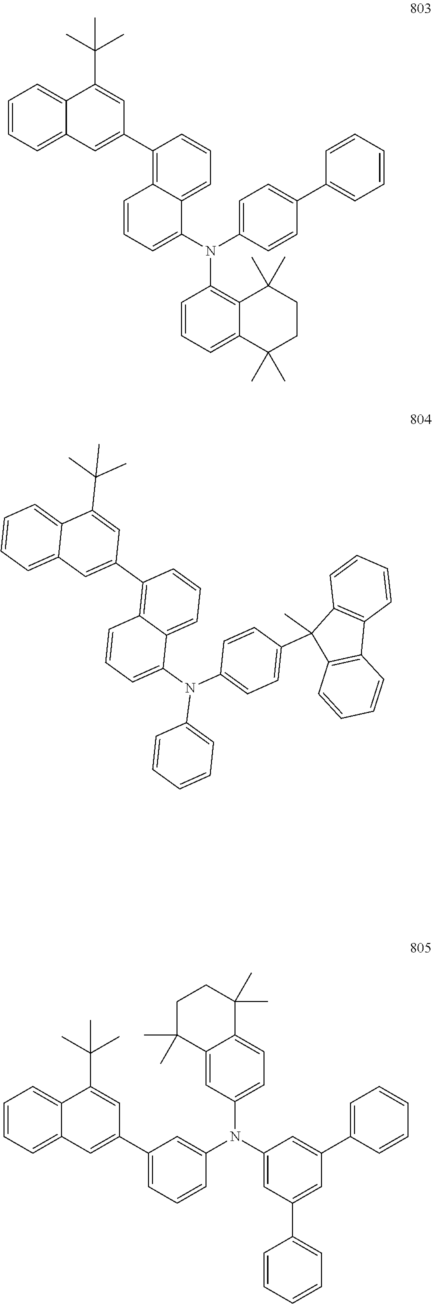 [SMILES: CC(C)(C)c1cc(-c2cccc3c(N(c4ccc(-c5ccccc5)cc4)c4cccc5c4C(C)(C)CCC5(C)C)cccc23)cc2ccccc12 ; CC(C)(C)c1cc(-c2cccc3c(N(c4ccccc4)c4ccc(C5(C)c6ccccc6-c6ccccc65)cc4)cccc23)cc2ccccc12 ; CC(C)(C)c1cc(-c2cccc(N(c3cc(-c4ccccc4)cc(-c4ccccc4)c3)c3ccc4c(c3)C(C)(C)CCC4(C)C)c2)cc2ccccc12]

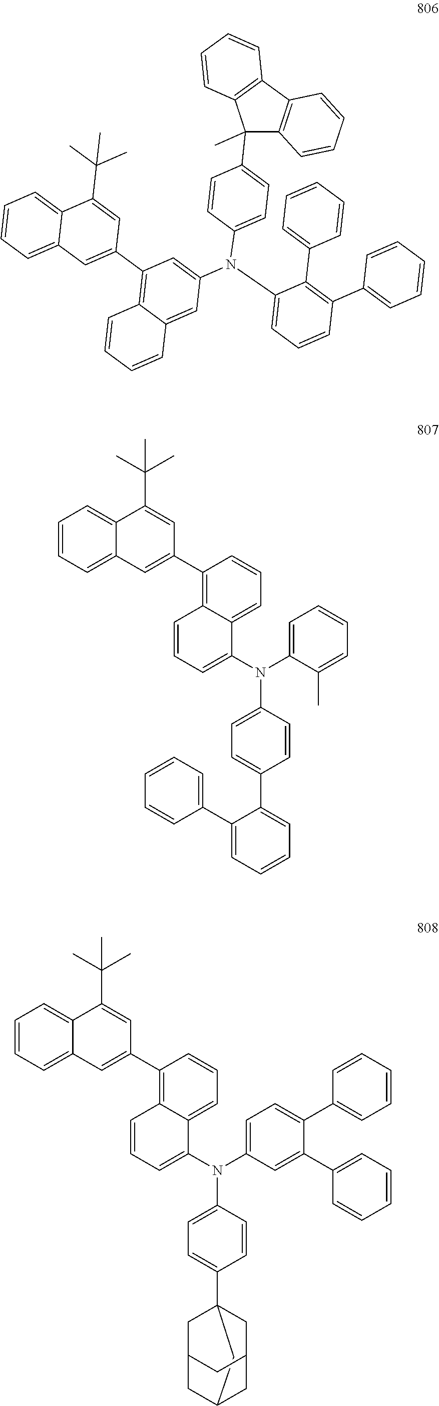 [SMILES: CC(C)(C)c1cc(-c2cc(N(c3ccc(C4(C)c5ccccc5-c5ccccc54)cc3)c3cccc(-c4ccccc4)c3-c3ccccc3)cc3ccccc23)cc2ccccc12 ; Cc1ccccc1N(c1ccc(-c2ccccc2-c2ccccc2)cc1)c1cccc2c(-c3cc(C(C)(C)C)c4ccccc4c3)cccc12 ; CC(C)(C)c1cc(-c2cccc3c(N(c4ccc(C56CC7CC(CC(C7)C5)C6)cc4)c4ccc(-c5ccccc5)c(-c5ccccc5)c4)cccc23)cc2ccccc12]

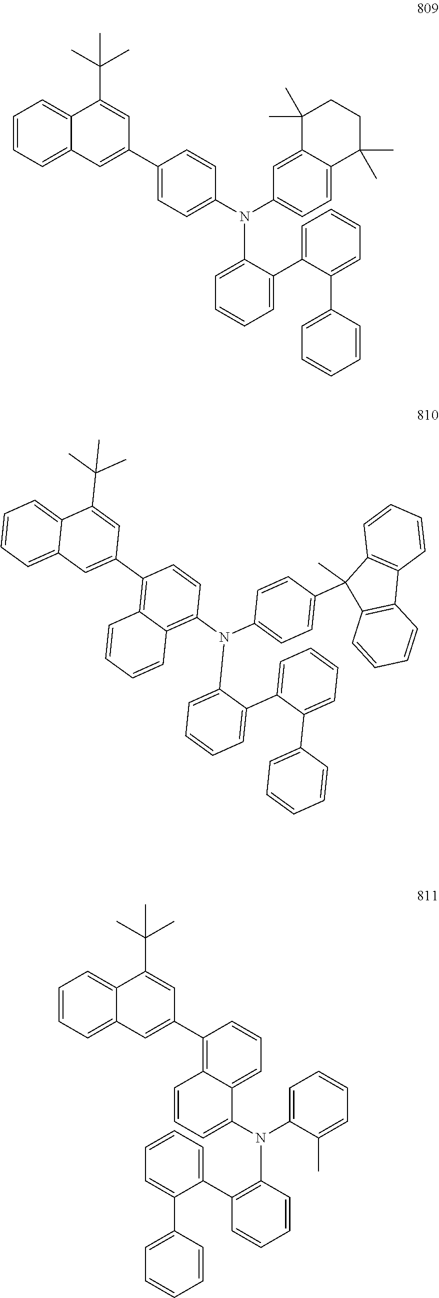 [SMILES: CC(C)(C)c1cc(-c2ccc(N(c3ccc4c(c3)C(C)(C)CCC4(C)C)c3ccccc3-c3ccccc3-c3ccccc3)cc2)cc2ccccc12 ; CC(C)(C)c1cc(-c2ccc(N(c3ccc(C4(C)c5ccccc5-c5ccccc54)cc3)c3ccccc3-c3ccccc3-c3ccccc3)c3ccccc23)cc2ccccc12 ; Cc1ccccc1N(c1ccccc1-c1ccccc1-c1ccccc1)c1cccc2c(-c3cc(C(C)(C)C)c4ccccc4c3)cccc12]

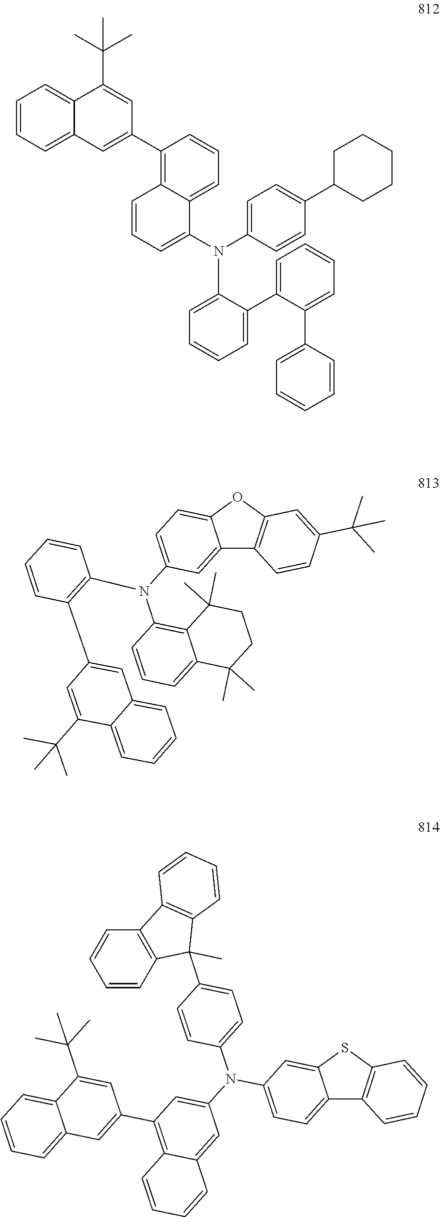 [SMILES: CC(C)(C)c1cc(-c2cccc3c(N(c4ccc(C5CCCCC5)cc4)c4ccccc4-c4ccccc4-c4ccccc4)cccc23)cc2ccccc12 ; CC(C)(C)c1ccc2c(c1)oc1ccc(N(c3ccccc3-c3cc(C(C)(C)C)c4ccccc4c3)c3cccc4c3C(C)(C)CCC4(C)C)cc12 ; CC(C)(C)c1cc(-c2cc(N(c3ccc(C4(C)c5ccccc5-c5ccccc54)cc3)c3ccc4c(c3)sc3ccccc34)cc3ccccc23)cc2ccccc12]

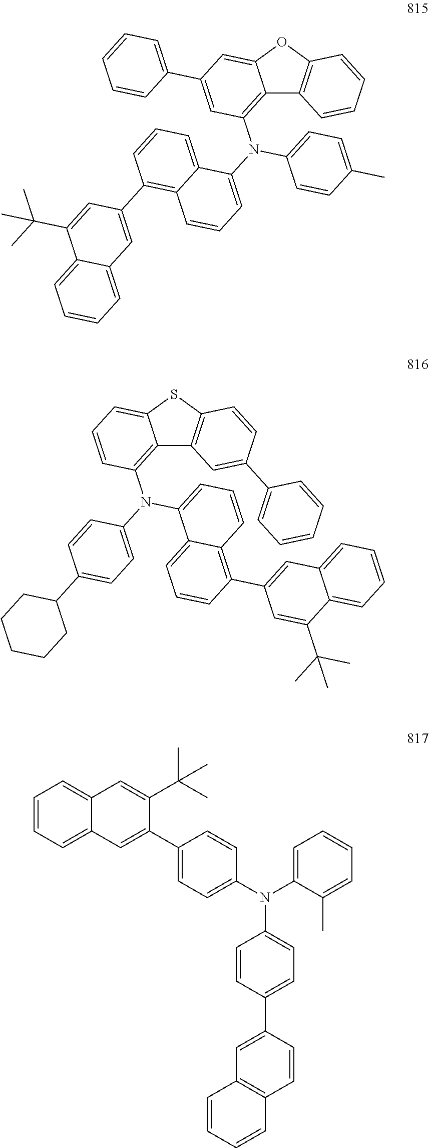 [SMILES: Cc1ccc(N(c2cccc3c(-c4cc(C(C)(C)C)c5ccccc5c4)cccc23)c2cc(-c3ccccc3)cc3oc4ccccc4c23)cc1 ; CC(C)(C)c1cc(-c2cccc3c(N(c4ccc(C5CCCCC5)cc4)c4cccc5sc6ccc(-c7ccccc7)cc6c45)cccc23)cc2ccccc12 ; Cc1ccccc1N(c1ccc(-c2ccc3ccccc3c2)cc1)c1ccc(-c2cc3ccccc3cc2C(C)(C)C)cc1]

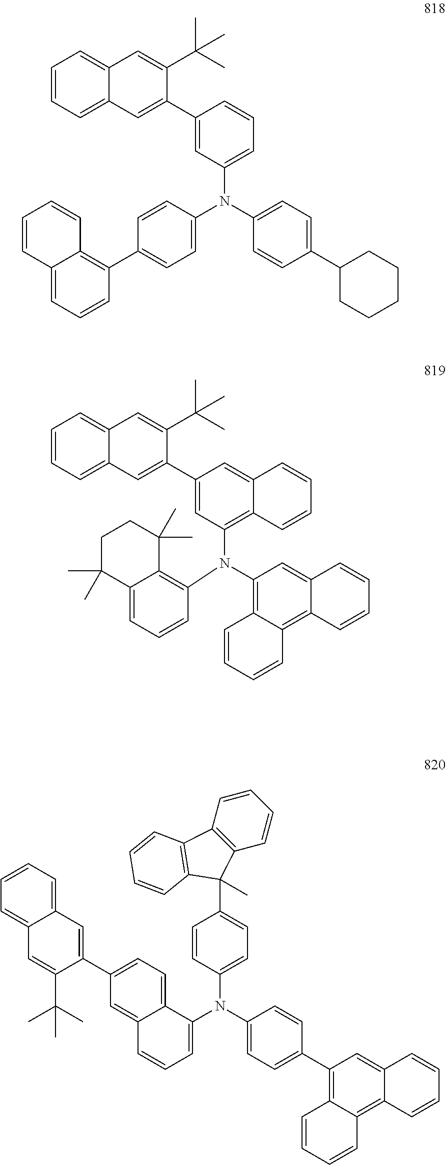 [SMILES: CC(C)(C)c1cc2ccccc2cc1-c1cccc(N(c2ccc(-c3cccc4ccccc34)cc2)c2ccc(C3CCCCC3)cc2)c1 ; CC(C)(C)c1cc2ccccc2cc1-c1cc(N(c2cccc3c2C(C)(C)CCC3(C)C)c2cc3ccccc3c3ccccc23)c2ccccc2c1 ; CC(C)(C)c1cc2ccccc2cc1-c1ccc2c(N(c3ccc(-c4cc5ccccc5c5ccccc45)cc3)c3ccc(C4(C)c5ccccc5-c5ccccc54)cc3)cccc2c1]

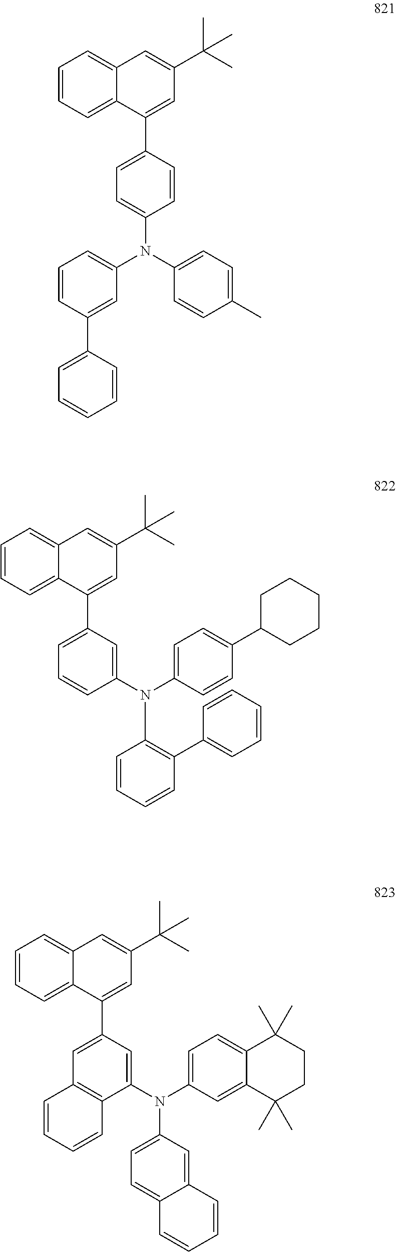 [SMILES: Cc1ccc(N(c2ccc(-c3cc(C(C)(C)C)cc4ccccc34)cc2)c2cccc(-c3ccccc3)c2)cc1 ; CC(C)(C)c1cc(-c2cccc(N(c3ccc(C4CCCCC4)cc3)c3ccccc3-c3ccccc3)c2)c2ccccc2c1 ; CC(C)(C)c1cc(-c2cc(N(c3ccc4c(c3)C(C)(C)CCC4(C)C)c3ccc4ccccc4c3)c3ccccc3c2)c2ccccc2c1]

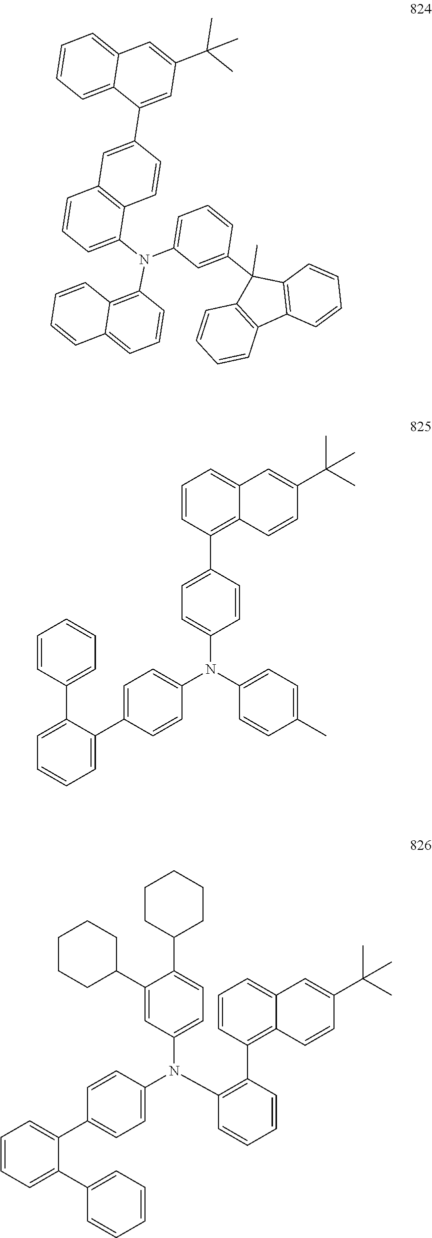 [SMILES: CC(C)(C)c1cc(-c2ccc3c(N(c4cccc(C5(C)c6ccccc6-c6ccccc65)c4)c4cccc5ccccc45)cccc3c2)c2ccccc2c1 ; Cc1ccc(N(c2ccc(-c3ccccc3-c3ccccc3)cc2)c2ccc(-c3cccc4cc(C(C)(C)C)ccc34)cc2)cc1 ; CC(C)(C)c1ccc2c(-c3ccccc3N(c3ccc(-c4ccccc4-c4ccccc4)cc3)c3ccc(C4CCCCC4)c(C4CCCCC4)c3)cccc2c1]

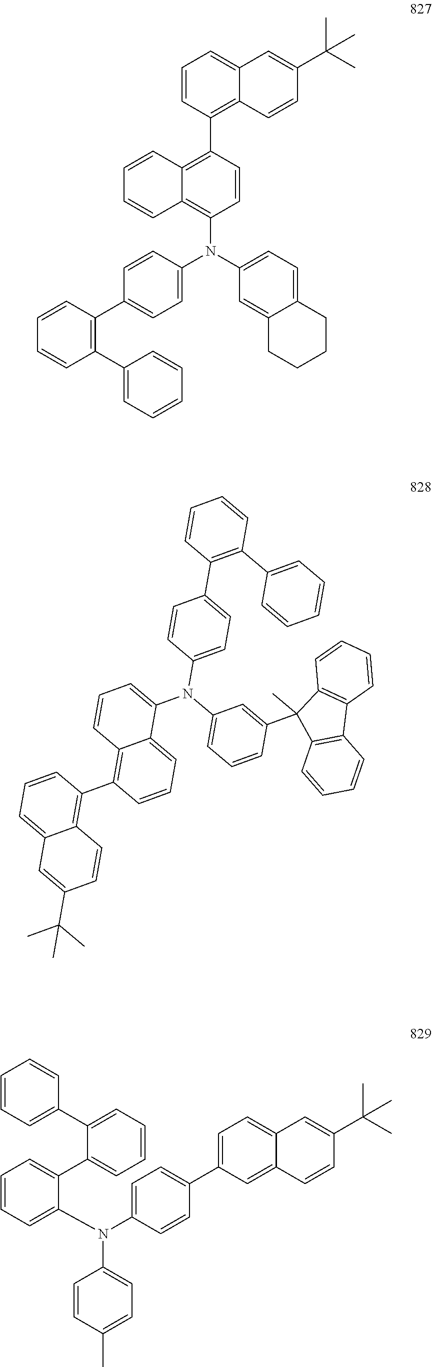 [SMILES: CC(C)(C)c1ccc2c(-c3ccc(N(c4ccc(-c5ccccc5-c5ccccc5)cc4)c4ccc5c(c4)CCCC5)c4ccccc34)cccc2c1 ; CC(C)(C)c1ccc2c(-c3cccc4c(N(c5ccc(-c6ccccc6-c6ccccc6)cc5)c5cccc(C6(C)c7ccccc7-c7ccccc76)c5)cccc34)cccc2c1 ; Cc1ccc(N(c2ccc(-c3ccc4cc(C(C)(C)C)ccc4c3)cc2)c2ccccc2-c2ccccc2-c2ccccc2)cc1]

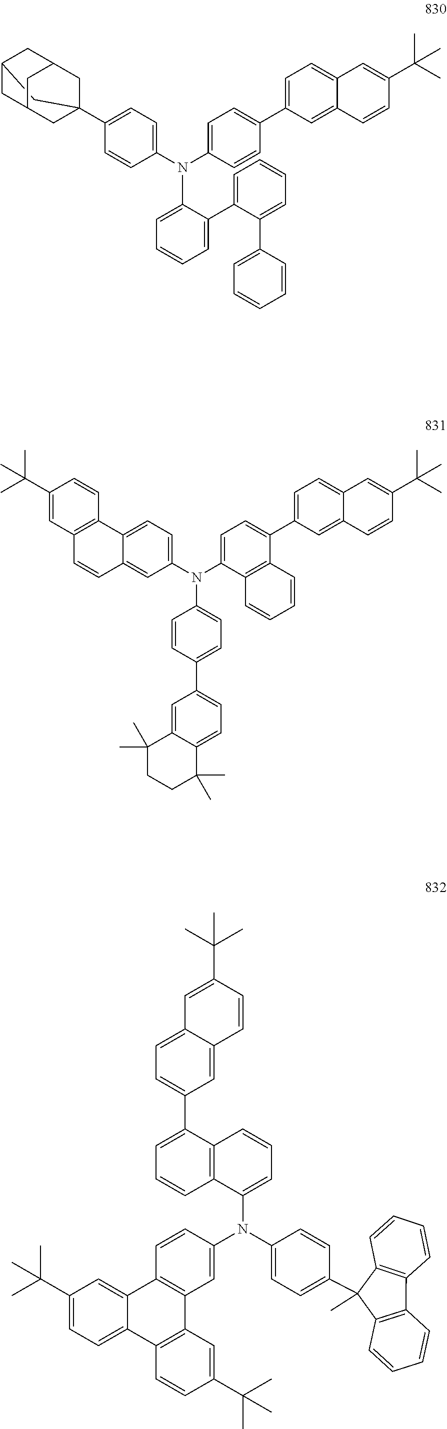

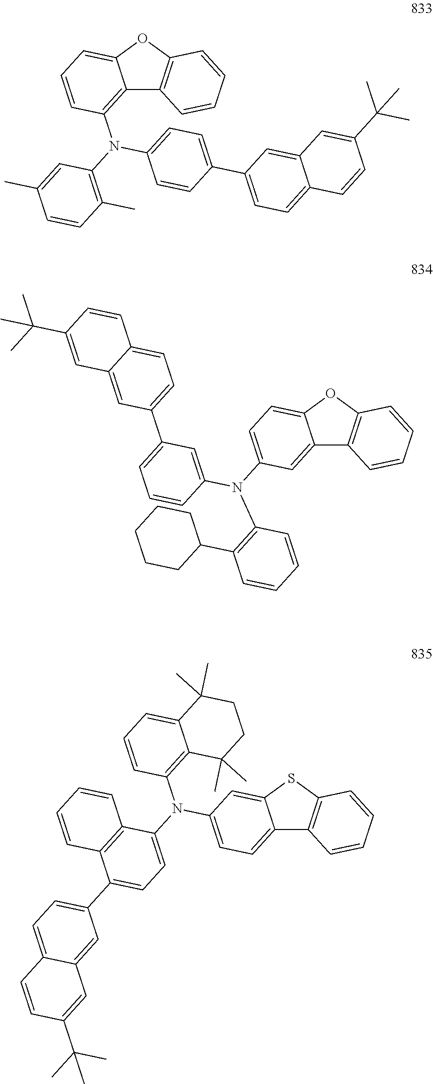

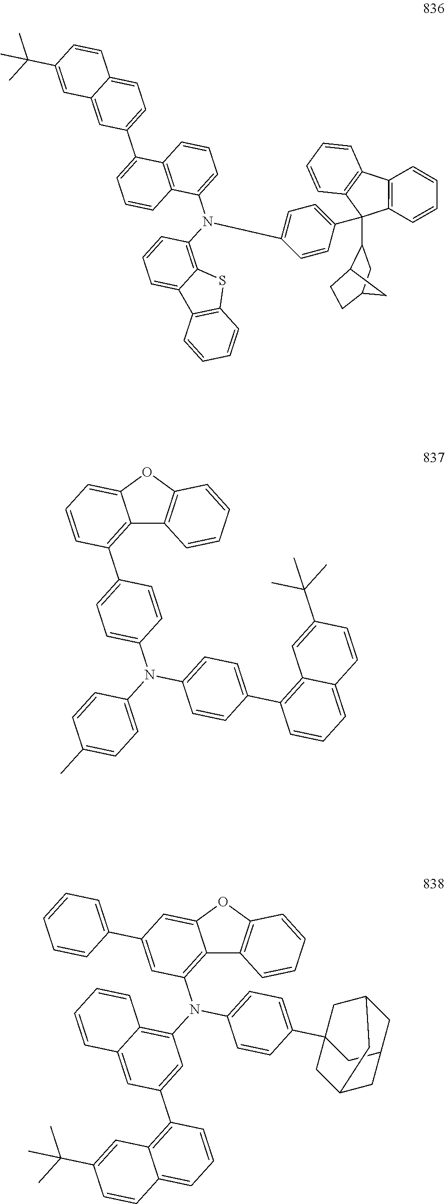 [SMILES: CC(C)(C)c1ccc2ccc(-c3cccc4c(N(c5ccc(C6(C7CC8CCC7C8)c7ccccc7-c7ccccc76)cc5)c5cccc6c5sc5ccccc56)cccc34)cc2c1 ; Cc1ccc(N(c2ccc(-c3cccc4ccc(C(C)(C)C)cc34)cc2)c2ccc(-c3cccc4oc5ccccc5c34)cc2)cc1 ; CC(C)(C)c1ccc2cccc(-c3cc(N(c4ccc(C56CC7CC(CC(C7)C5)C6)cc4)c4cc(-c5ccccc5)cc5oc6ccccc6c45)c4ccccc4c3)c2c1]

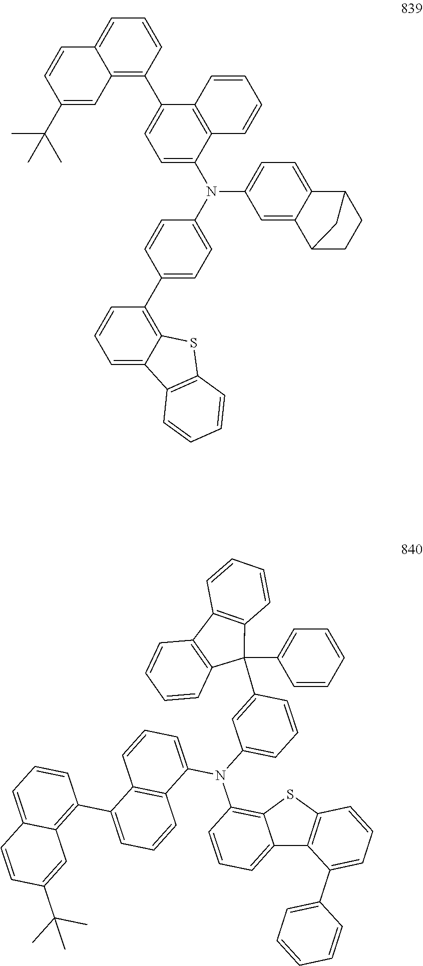 [SMILES: CC(C)(C)c1ccc2cccc(-c3ccc(N(c4ccc(-c5cccc6c5sc5ccccc56)cc4)c4ccc5c(c4)C4CCC5C4)c4ccccc34)c2c1 ; CC(C)(C)c1ccc2cccc(-c3cccc4c(N(c5cccc(C6(c7ccccc7)c7ccccc7-c7ccccc76)c5)c5cccc6c5sc5cccc(-c7ccccc7)c56)cccc34)c2c1]

In an exemplary embodiment of the present application, the above compounds are only exemplary and other compounds containing additional substituents included in Chemical Formula 1 may also be included. In addition, the position of substitution of deuterium in the compound may be excluded from a specific position during the deuterium substitution and synthesis process, and hydrogen and deuterium may exist together.

In addition, by introducing various substituents to the structure of Chemical Formula 1, compounds having the unique characteristics of the introduced substituents can be synthesized. For example, by introducing substituents mainly used in a hole injection material, a hole transport material, a light-emitting material, an electron transport material and an electron injection material during the manufacturing of an organic light-emitting device to the core structure, a material satisfying the conditions required for each organic layer may be synthesized.

In addition, the band gap may be controlled finely by introducing various substituents to the structure of Chemical Formula 1 or by changing the bonding positions, and on the other hand, the characteristics at the interface between organic layers can be improved.

In addition, the compound of Chemical Formula 1 has superior thermal stability, which provides operating stability to the organic light-emitting device and improves lifetime characteristics.

In an exemplary embodiment of the present application, there is provided an organic light-emitting device including a first electrode; a second electrode facing the first electrode; and one or more organic layer provided between the first electrode and the second electrode, wherein one or more layer of the organic layer contains the compound represented by Chemical Formula 1 described above.

In another exemplary embodiment, there is provided an organic light-emitting device including a first electrode; a second electrode facing the first electrode; and one or more organic layer provided between the first electrode and the second electrode, wherein one or more layer of the organic layer contains one compound represented by Chemical Formula 1 described above.

In another exemplary embodiment, there is provided an organic light-emitting device including a first electrode; a second electrode facing the first electrode; and one or more organic layer provided between the first electrode and the second electrode, wherein one or more layer of the organic layer contains two or more compounds represented by Chemical Formula 1 described above.

In another exemplary embodiment, the compound represented by Chemical Formula 1 may be used as a light-emitting material in a light-emitting layer of the organic light-emitting device.

In another exemplary embodiment, the compound represented by Chemical Formula 1 may be used as a host material in a light-emitting layer of the organic light-emitting device.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light-emitting device may be a blue organic light-emitting device, and the compound according to Chemical Formula 1 may be used as a material of the blue organic light-emitting device.

In an exemplary embodiment of the present application, the organic light-emitting device may be a green organic light-emitting device, and the compound represented by Chemical Formula 1 may be used as a material of the green organic light-emitting device.

In an exemplary embodiment of the present application, the organic light-emitting device may be a red organic light-emitting device, and the compound represented by Chemical Formula 1 may be used as a material of the red organic light-emitting device.

In an exemplary embodiment of the present application, the organic light-emitting device may be a blue organic light-emitting device, and the compound according to Chemical Formula 1 may be used as a light-emitting layer material of the blue organic light-emitting device.

In an exemplary embodiment of the present application, the organic light-emitting device may be a green organic light-emitting device, and the compound represented by Chemical Formula 1 may be used as a light-emitting layer material of the green organic light-emitting device.

In an exemplary embodiment of the present application, the organic light-emitting device may be a red organic light-emitting device, and the compound represented by Chemical Formula 1 may be used as a light-emitting layer material of the red organic light-emitting device.

In an exemplary embodiment of the present application, the details of the compound represented by Chemical Formula 1 are those described above.

The organic light-emitting device of the present disclosure may be manufactured using a conventional organic light-emitting device manufacturing method and materials, except that one or more organic layer is formed using the compound described above.

The compound may be formed into an organic layer during the manufacturing of the organic light-emitting device, not only by a vacuum deposition method but also by a solution coating method. Here, the solution coating method means spin coating, dip coating, inkjet printing, screen printing, spraying, roll coating, etc., although not being limited thereto.

The organic layer of the organic light-emitting device of the present disclosure may have a single-layer structure, but may also have a multi-layer structure in which two or more organic layers are stacked. For example, the organic light-emitting device of the present disclosure may have a structure including a hole injection layer, a hole transport layer, a light-emitting layer, an electron transport layer, an electron injection layer, etc. as the organic layer. However, the structure of the organic light-emitting device is not limited thereto, and may include a smaller number of organic layers.

In an exemplary embodiment of the present application, the organic layer may include one or more layer selected from a group consisting of a light-emitting layer, a hole injection layer, a hole transport layer, a hole transport auxiliary layer, an electron blocking layer, a hole blocking layer, an electron transport layer and an electron injection layer.

In an exemplary embodiment of the present application, a green phosphorescent dopant, Ir(ppy)₃, may be used as an iridium-based dopant.

In an exemplary embodiment of the present application, a red phosphorescent dopant, (piq)₂(Ir)(acac), may be used as an iridium-based dopant.

In an exemplary embodiment of the present application, a pyrene-based dopant may be used as a blue phosphorescent dopant.

In an exemplary embodiment of the present application, the organic layer of the organic light-emitting device includes a light-emitting layer, and the light-emitting layer contains the compound described above.

In an exemplary embodiment of the present application, the organic layer of the organic light-emitting device includes a light-emitting layer, the light-emitting layer contains a host material, and the host material includes the compound described above.

In the organic light-emitting device of the present disclosure, the organic layer may include an electron injection layer or an electron transport layer, and the electron injection layer or the electron transport layer may contain the compound described above.

In another organic light-emitting device, the organic layer may include a hole blocking layer, and the hole blocking layer may contain the compound described above.

In another organic light-emitting device, the organic layer may include an electron blocking layer, and the electron blocking layer may contain the compound described above.

In another organic light-emitting device, the organic layer may include a hole transport layer, a light-emitting layer or an electron blocking layer, and the hole transport layer, the light-emitting layer or the electron blocking layer may contain the compound described above.

In another organic light-emitting device, the organic layer may include a hole transport layer or a hole transport auxiliary layer, and the hole transport layer or the hole transport auxiliary layer may contain the compound described above.

In another organic light-emitting device, the organic layer may include a hole transport layer, and the hole transport layer may contain the compound described above.

In the organic light-emitting device of the present application, a material with a relatively high work function may be used as the positive electrode material. A transparent conductive oxide, a metal, a conductive polymer, etc. may be used. Specific examples of the positive electrode material include a metal such as vanadium, chromium, copper, zinc or gold, or an alloy thereof; a metal oxide such as zinc oxide, indium oxide, indium tin oxide (ITO) or indium zinc oxide (IZO); a combination of a metal and an oxide such as ZnO:Al or SnO₂:Sb; a conductive polymer such as poly(3-methylthiophene), poly[3,4-(ethylene-1,2-dioxy)thiophene] (PEDOT), polypyrrole or polyaniline, etc., although not being limited thereto.

As the negative electrode material, a material with a relatively low work function may be used. A metal, a metal oxide, a conductive polymer, etc. may be used. Specific examples of the negative electrode material include a metal such as magnesium, calcium, sodium, potassium, titanium, indium, yttrium, lithium, gadolinium, aluminum, silver, tin or lead, or an alloy thereof; a multi-layer structured material such as LiF/Al or LiO₂/Al, etc., although not being limited thereto.

As the hole injection material, a known hole injection material may be used. For example, a phthalocyanine compound such as copper phthalocyanine, etc. disclosed in U.S. Pat. No. 4,356,429, a Starburst-type amine derivative, e.g., tris(4-carbazol-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) disclosed in the literature [Advanced Material, 6, p. 677 (1994)], a soluble conductive polymer such as polyaniline/dodecylbenzenesulfonic acid, poly(3,4-ethylenedioxythiophene)/poly(4-styrenesulfonate), polyaniline/camphorsulfonic acid or polyaniline/poly(4-styrenesulfonate), etc. may be used.

As the hole transport material, a pyrazoline derivative, an arylamine-based derivative, a stilbene derivative, a triphenyldiamine derivative, etc. may be used, and a low-molecular-weight or high-molecular-weight material may also be used.

As the electron transport material, a metal complex of an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, 8-hydroxyquinoline and a derivative thereof, etc. may be used. Not only a low-molecular-weight material but also a high-molecular-weight material may be used.

As the electron injection material, LiF is typically used in the art, for example, although the present application is not limited thereto.

As the light-emitting material, a red, green or blue light-emitting material may be used and, if necessary, a mixture of two or more light-emitting materials may be used. In this case, two or more light-emitting materials may be deposited using individual supply sources or may be deposited using a single supply source after being premixed. In addition, a fluorescent material or a phosphorescent material may be used as the light-emitting material. As the light-emitting material, a material that emits light as holes and electrons injected respectively from the positive electrode and the negative electrode are recombined may be used alone. However, a host material and a dopant material that participate in light emission may also be used together.

When a mixed host is used as the light-emitting material, hosts of the same class may be mixed, or hosts of different classes may be mixed. For example, two or more types of materials from n-type host materials or p-type host materials may be selected and used as the host material of the light-emitting layer.

The organic light-emitting device according to an exemplary embodiment of the present application may be of a front emission, back emission, or double-sided emission type depending on the material used.

The hetero ring compound according to an exemplary embodiment of the present application may operate in organic electronic devices including organic solar cells, organic photoconductors, organic transistors, etc. on a principle similar to that applied to organic light-emitting devices.

The organic light-emitting device of the present disclosure may further include one or more layer selected from a group consisting of a light-emitting layer, a hole injection layer, a hole transport layer, a hole transport auxiliary layer, an electron blocking layer, a hole blocking layer, an electron transport layer and an electron injection layer.

The organic light-emitting device of the present disclosure may further include one or more layer selected from a group consisting of a light-emitting layer, a hole injection layer, a hole transport layer, an electron blocking layer, a hole blocking layer, an electron transport layer and an electron injection layer.

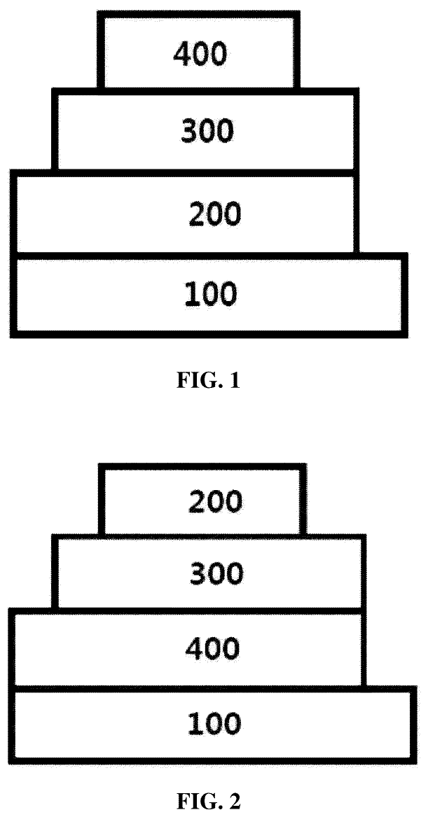

FIGS. 1 to 4 illustrate the stacking order of electrodes and organic layers in organic light-emitting devices according to exemplary embodiments of the present application. However, the drawings are not intended to limit the scope of the present application, and the structures of organic light-emitting devices known in the art can also be applied to the present application.

FIG. 1 shows an organic light-emitting device in which a positive electrode 200, an organic layer 300 and a negative electrode 400 are stacked sequentially on a substrate 100. However, without being limited to such a structure, an organic light-emitting device in which a negative electrode, an organic layer and a positive electrode are stacked sequentially on a substrate is also possible, as shown in FIG. 2.

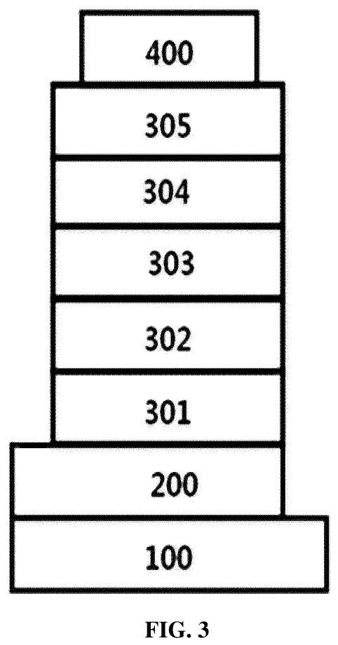

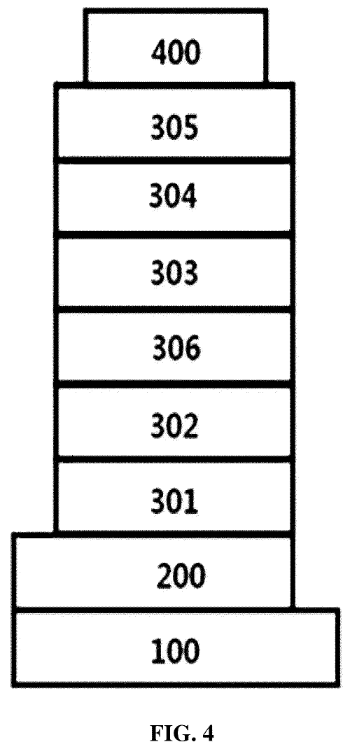

FIGS. 3 and 4 illustrate the cases where the organic layer consists of multiple layers.

The organic light-emitting device shown in FIG. 3 includes a hole injection layer 301, a hole transport layer 302, a light-emitting layer 303, an electron transport layer 304 and an electron injection layer 305.

The organic light-emitting device shown in FIG. 4 includes a hole injection layer 301, a hole transport layer 302, an electron blocking layer 306, a light-emitting layer 303, an electron transport layer 304 and an electron injection layer 305.

However, the scope of the present application is not limited by the stacking structures. Other layers except the light-emitting layer may be omitted, if necessary, and other necessary functional layers may be further added.

The organic layer containing the compound of Chemical Formula 1 may further contain other materials as desired.

Hereinafter, the present specification will be described in more detail through examples. However, these examples are only intended to illustrate the present application, and are not intended to limit the scope of the present application.

### SYNTHESIS EXAMPLES

### [Preparation Example 1] Preparation of Compound 1

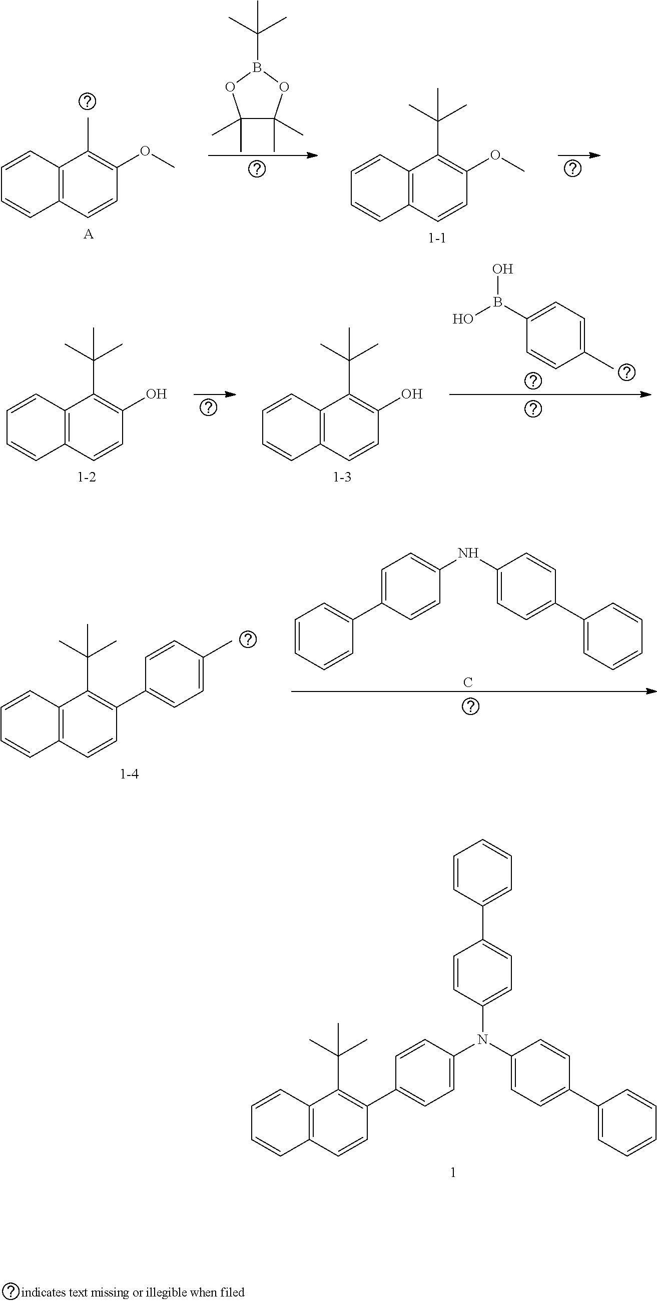

### 1) Preparation of Compound 1-1

1-Bromo-2-methoxynaphthalene (A) (30.0 g, 0.126 mol, 1.0 eq), 2-(1,1-dimethylethyl)-4,4,5,5-tetramethyl-1,3,2-dioxaborolane (28.5 g, 0.152 mol, 1.2 eq), Pd(PPh₃)₄ (7.3 g 0.006 mol, 0.05 eq), K₂CO₃ (35.1 g, 0.253 mol, 2.0 eq), 1,4-dioxane (360 mL) and water (90 mL) were stirred at 100° C. for 12 hours. After terminating reaction by adding water, extraction was performed by adding methylene chloride (MC) and water. Then, moisture was removed with MgSO₄. Compound 1-1 (21 g, 77% yield) was obtained by separating with a silica gel column.

### 2) Preparation of Compound 1-2

Compound 1-1 (21 g, 0.098 mol, 1 eq) and MC (210 mL) were stirred at 0° C. Then, after slowly adding BBr₃ (63.4 g, 0.253 mol, 2.0 eq) dropwise, the mixture was stirred at room temperature for 1 hour. After terminating reaction by adding water, extraction was performed using MC and water. Then, moisture was removed with MgSO₄. Compound 1-2 (18.8 g, 95% yield) was obtained by separating with a silica gel column.

### 3) Preparation of Compound 1-3

Compound 1-2 (18 g, 0.090 mol, 1 eq), triethylamine (10.9 g, 0.107 mol, 1.2 eq) and MC (200 mL) were stirred at 0° C. Then, after slowly adding trifluoromethanesulfonic anhydride (30.5 g, 0.107 mol, 1.2 eq) dropwise, the mixture was stirred at room temperature for 2 hours. After terminating reaction by adding water, extraction was performed using MC and water. Then, moisture was removed with MgSO₄. Compound 1-3 (27.6 g, 92% yield) was obtained by separating with a silica gel column.

### 4) Preparation of Compound 1-4

Compound 1-3 (27 g, 0.081 mol, 1 eq), (4-chlorophenyl) boronic acid (B) (14.3 g, 0.089 mol, 1.1 eq), Pd(PPh₃)₄ (4.7 g 0.004 mol, 0.05 eq), K₂CO₃ (22.4 g, 0.162 mol, 2.0 eq), 1,4-dioxane (280 mL) and water (70 mL) were stirred at 100° C. for 3 hours. After terminating reaction by adding water, extraction was performed using MC and water. Then, moisture was removed with MgSO₄. Compound 1-4 (22.4 g, 93% yield) was obtained by separating with a silica gel column.

### 5) Preparation of Compound 1

Compound 1-4 (10 g, 0.034 mol, 1 eq), bis(4-biphenylyl)amine (C) (12.2 g, 0.037 mol, 1.1 eq), Pd₂(dba)₃ (1.5 g 0.002 mol, 0.05 eq), XPhos (1.6 g, 0.003 mol, 0.1 eq), NaOt-Bu (6.52 g, 0.068 mol, 2.0 eq) and toluene (80 mL) were stirred at 100° C. for 2 hours. After terminating reaction by adding water, extraction was performed using MC and water. Then, moisture was removed with MgSO₄. Compound 1 (16.1 g, 51% yield) was obtained by separating with a silica gel column.

Compounds were synthesized in the same manner as in Preparation Example 1, except that intermediate A described in Table 1 was used instead of 1-bromo-2-methoxynaphthalene (A), intermediate B was used instead of (4-chlorophenyl) boronic acid (B) and, intermediate C was used instead of bis(4-biphenylyl)amine (C).

TABLE 1

| Com- | | | | |
|---|---|---|---|---|
| pound | | | | |
| No. | Intermediate A | Intermediate B | Intermediate C | Yiled |
| 1 | 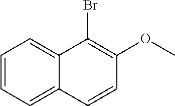 | 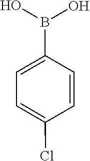 | 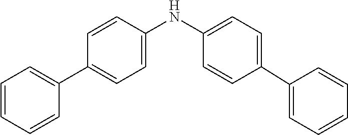 [SMILES: c1ccc(-c2ccc(Nc3ccc(-c4ccccc4)cc3)cc2)cc1] | 51% |
| 36 | 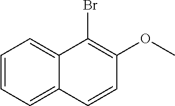 | 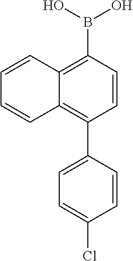 [SMILES: OB(O)c1ccc(-c2ccc(Cl)cc2)c2ccccc12] | 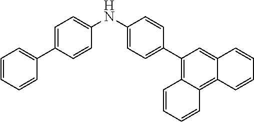 [SMILES: c1ccc(-c2ccc(Nc3ccc(-c4cc5ccccc5c5ccccc45)cc3)cc2)cc1] | 48% |
| 54 | 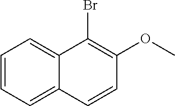 | 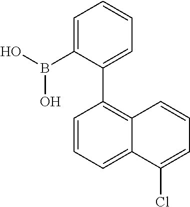 [SMILES: OB(O)c1ccccc1-c1cccc2c(Cl)cccc12] | 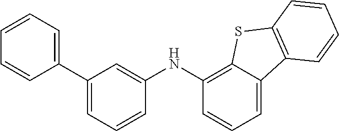 [SMILES: c1ccc(-c2cccc(Nc3cccc4c3sc3ccccc34)c2)cc1] | 47% |
| 62 | 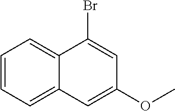 | 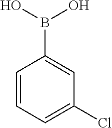 | 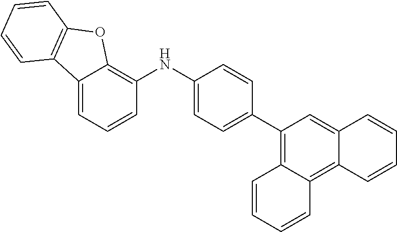 [SMILES: c1ccc2c(c1)cc(-c1ccc(Nc3cccc4c3oc3ccccc34)cc1)c1ccccc12] | 49% |
| 96 | 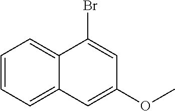 | 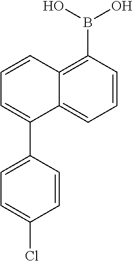 [SMILES: OB(O)c1cccc2c(-c3ccc(Cl)cc3)cccc12] | 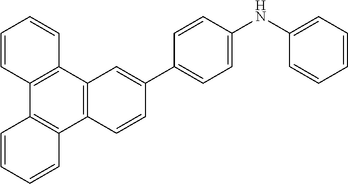 [SMILES: c1ccc(Nc2ccc(-c3ccc4c5ccccc5c5ccccc5c4c3)cc2)cc1] | 48% |
| 108 | 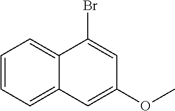 | 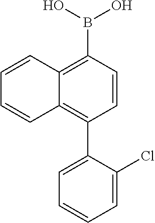 [SMILES: OB(O)c1ccc(-c2ccccc2Cl)c2ccccc12] | 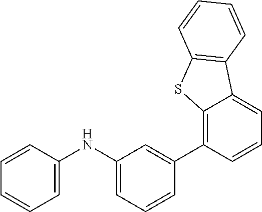 [SMILES: c1ccc(Nc2cccc(-c3cccc4c3sc3ccccc34)c2)cc1] | 48% |
| 120 | 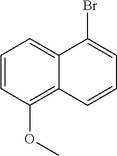 | 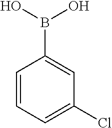 | 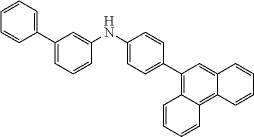 [SMILES: c1ccc(-c2cccc(Nc3ccc(-c4cc5ccccc5c5ccccc45)cc3)c2)cc1] | 50% |
| 130 | 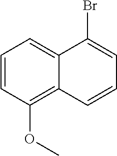 | 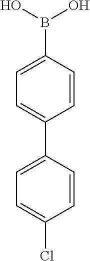 [SMILES: OB(O)c1ccc(-c2ccc(Cl)cc2)cc1] | 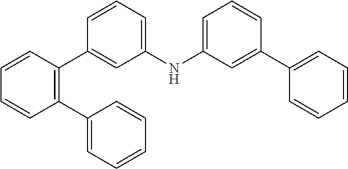 [SMILES: c1ccc(-c2cccc(Nc3cccc(-c4ccccc4-c4ccccc4)c3)c2)cc1] | 50% |
| 142 | 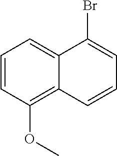 | 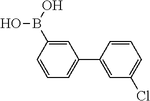 [SMILES: OB(O)c1cccc(-c2cccc(Cl)c2)c1] | 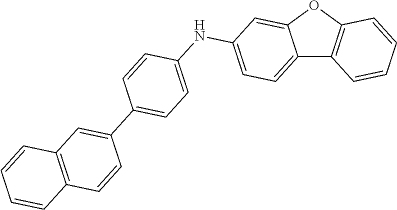 [SMILES: c1ccc2cc(-c3ccc(Nc4ccc5c(c4)oc4ccccc45)cc3)ccc2c1] | 48% |
| 171 | 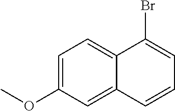 | 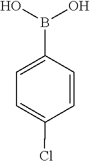 | 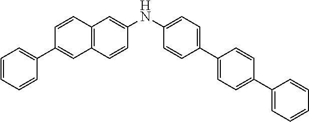 [SMILES: c1ccc(-c2ccc(-c3ccc(Nc4ccc5cc(-c6ccccc6)ccc5c4)cc3)cc2)cc1] | 49% |
| 178 | 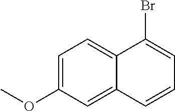 | 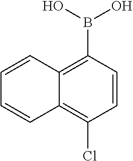 [SMILES: OB(O)c1ccc(Cl)c2ccccc12] | 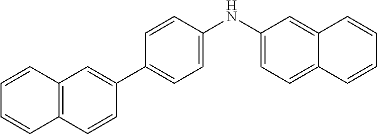 [SMILES: c1ccc2cc(Nc3ccc(-c4ccc5ccccc5c4)cc3)ccc2c1] | 47% |
| 223 | 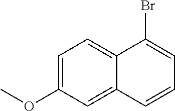 | 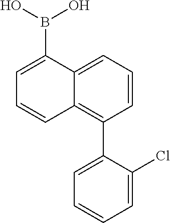 [SMILES: OB(O)c1cccc2c(-c3ccccc3Cl)cccc12] | 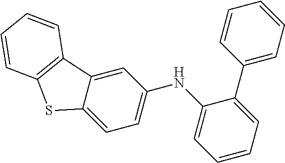 [SMILES: c1ccc(-c2ccccc2Nc2ccc3sc4ccccc4c3c2)cc1] | 48% |
| 234 | 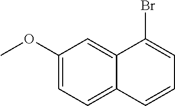 | 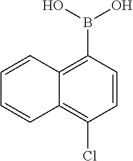 [SMILES: OB(O)c1ccc(Cl)c2ccccc12] | 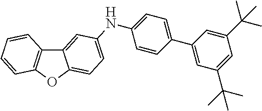 [SMILES: CC(C)(C)c1cc(-c2ccc(Nc3ccc4oc5ccccc5c4c3)cc2)cc(C(C)(C)C)c1] | 48% |
| 249 | 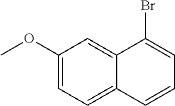 | 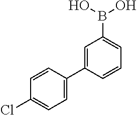 [SMILES: OB(O)c1cccc(-c2ccc(Cl)cc2)c1] | 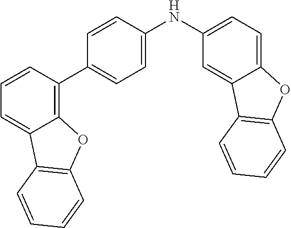 [SMILES: c1ccc2c(c1)oc1ccc(Nc3ccc(-c4cccc5c4oc4ccccc45)cc3)cc12] | 49% |
| 261 | 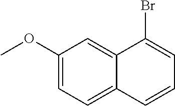 | 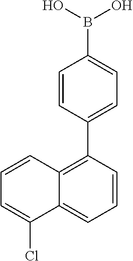 [SMILES: OB(O)c1ccc(-c2cccc3c(Cl)cccc23)cc1] | 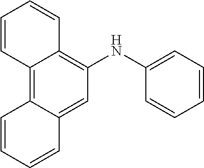 [SMILES: c1ccc(Nc2cc3ccccc3c3ccccc23)cc1] | 47% |
| 288 | 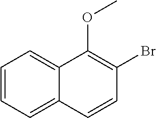 | 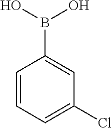 | 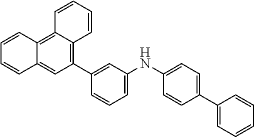 [SMILES: c1ccc(-c2ccc(Nc3cccc(-c4cc5ccccc5c5ccccc45)c3)cc2)cc1] | 50% |
| 293 | 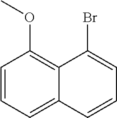 | 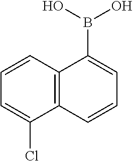 [SMILES: OB(O)c1cccc2c(Cl)cccc12] | 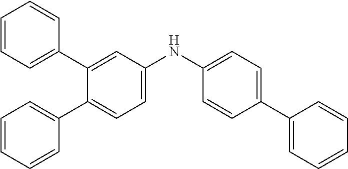 [SMILES: c1ccc(-c2ccc(Nc3ccc(-c4ccccc4)c(-c4ccccc4)c3)cc2)cc1] | 47% |
| 303 | 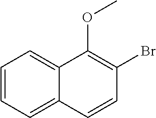 | 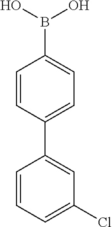 [SMILES: OB(O)c1ccc(-c2cccc(Cl)c2)cc1] | 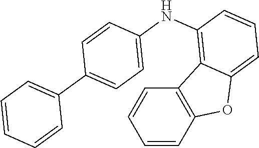 [SMILES: c1ccc(-c2ccc(Nc3cccc4oc5ccccc5c34)cc2)cc1] | 49% |
| 305 | 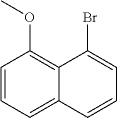 | 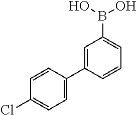 [SMILES: OB(O)c1cccc(-c2ccc(Cl)cc2)c1] | 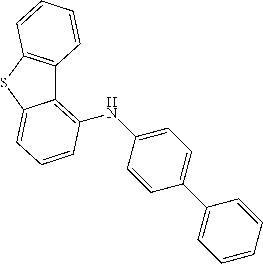 [SMILES: c1ccc(-c2ccc(Nc3cccc4sc5ccccc5c34)cc2)cc1] | 49% |
| 325 | 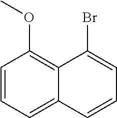 | 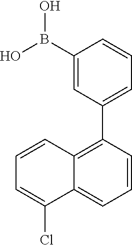 [SMILES: OB(O)c1cccc(-c2cccc3c(Cl)cccc23)c1] | 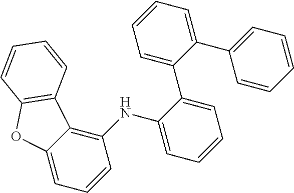 [SMILES: c1ccc(-c2ccccc2-c2ccccc2Nc2cccc3oc4ccccc4c23)cc1] | 48% |
| 331 | 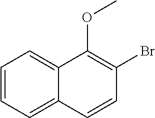 | 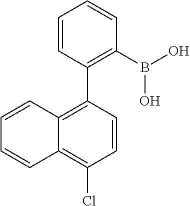 [SMILES: OB(O)c1ccccc1-c1ccc(Cl)c2ccccc12] | 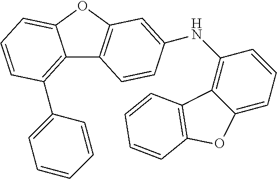 [SMILES: c1ccc(-c2cccc3oc4cc(Nc5cccc6oc7ccccc7c56)ccc4c23)cc1] | 50% |
| 358 | 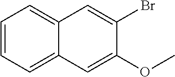 | 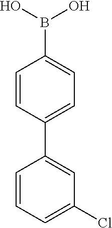 [SMILES: OB(O)c1ccc(-c2cccc(Cl)c2)cc1] | 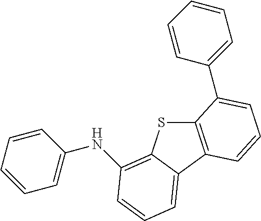 [SMILES: c1ccc(Nc2cccc3c2sc2c(-c4ccccc4)cccc23)cc1] | 48% |
| 380 | 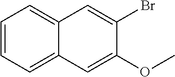 | 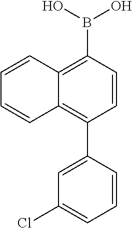 [SMILES: OB(O)c1ccc(-c2cccc(Cl)c2)c2ccccc12] | 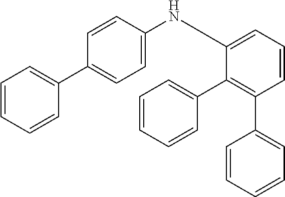 [SMILES: c1ccc(-c2ccc(Nc3cccc(-c4ccccc4)c3-c3ccccc3)cc2)cc1] | 48% |
| 383 | 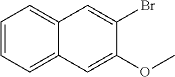 | 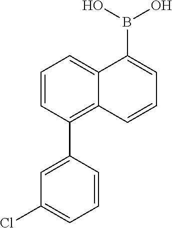 [SMILES: OB(O)c1cccc2c(-c3cccc(Cl)c3)cccc12] | 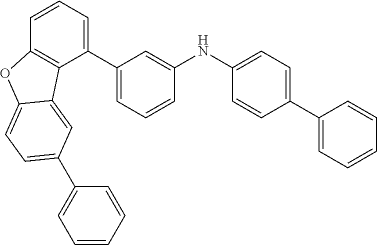 [SMILES: c1ccc(-c2ccc(Nc3cccc(-c4cccc5oc6ccc(-c7ccccc7)cc6c45)c3)cc2)cc1] | 49% |
| 396 | 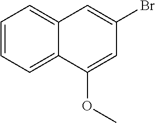 | 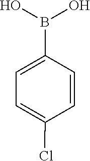 | 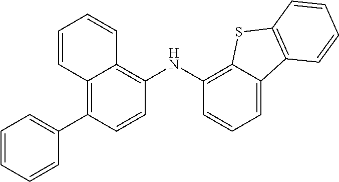 [SMILES: c1ccc(-c2ccc(Nc3cccc4c3sc3ccccc34)c3ccccc23)cc1] | 49% |
| 403 | 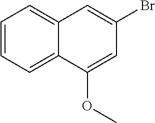 | 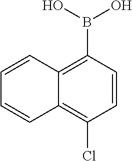 [SMILES: OB(O)c1ccc(Cl)c2ccccc12] | 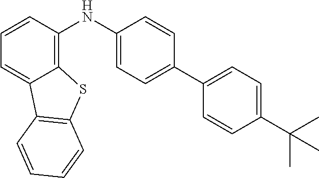 [SMILES: CC(C)(C)c1ccc(-c2ccc(Nc3cccc4c3sc3ccccc34)cc2)cc1] | 48% |
| 426 | 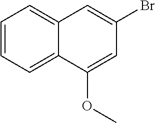 | 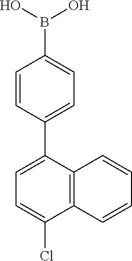 [SMILES: OB(O)c1ccc(-c2ccc(Cl)c3ccccc23)cc1] | 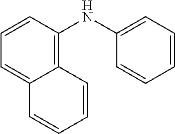 [SMILES: c1ccc(Nc2cccc3ccccc23)cc1] | 49% |
| 455 | 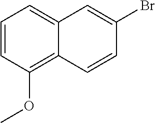 | 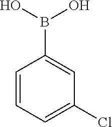 | 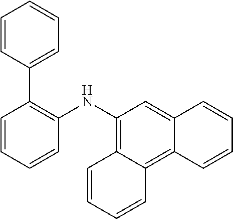 [SMILES: c1ccc(-c2ccccc2Nc2cc3ccccc3c3ccccc23)cc1] | 47% |
| 480 | 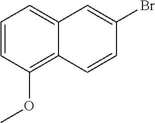 | 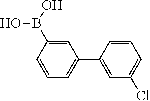 [SMILES: OB(O)c1cccc(-c2cccc(Cl)c2)c1] | 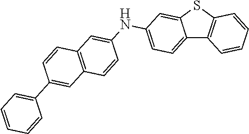 [SMILES: c1ccc(-c2ccc3cc(Nc4ccc5c(c4)sc4ccccc45)ccc3c2)cc1] | 49% |
| 489 | 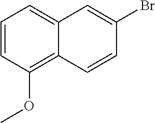 | 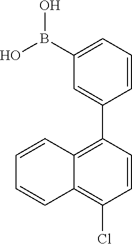 [SMILES: OB(O)c1cccc(-c2ccc(Cl)c3ccccc23)c1] | 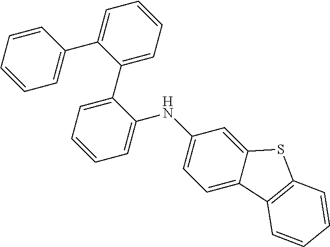 [SMILES: c1ccc(-c2ccccc2-c2ccccc2Nc2ccc3c(c2)sc2ccccc23)cc1] | 49% |
| 508 | 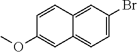 | 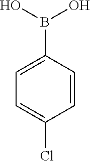 | 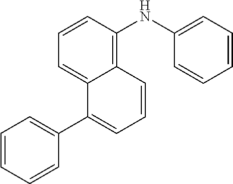 [SMILES: c1ccc(Nc2cccc3c(-c4ccccc4)cccc23)cc1] | 48% |
| 518 | 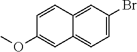 | 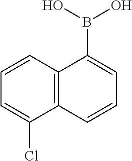 [SMILES: OB(O)c1cccc2c(Cl)cccc12] | 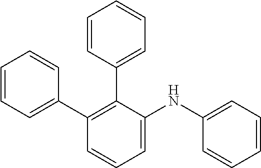 [SMILES: c1ccc(Nc2cccc(-c3ccccc3)c2-c2ccccc2)cc1] | 49% |
| 559 | 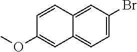 | 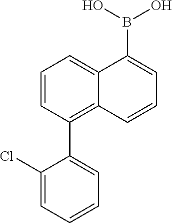 [SMILES: OB(O)c1cccc2c(-c3ccccc3Cl)cccc12] | 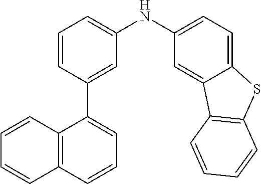 [SMILES: c1cc(Nc2ccc3sc4ccccc4c3c2)cc(-c2cccc3ccccc23)c1] | 48% |
| 573 | 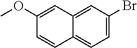 | 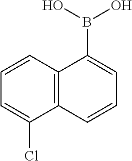 [SMILES: OB(O)c1cccc2c(Cl)cccc12] | 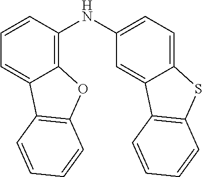 [SMILES: c1ccc2c(c1)oc1c(Nc3ccc4sc5ccccc5c4c3)cccc12] | 49% |
| 590 | 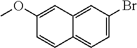 | 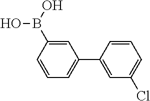 [SMILES: OB(O)c1cccc(-c2cccc(Cl)c2)c1] | 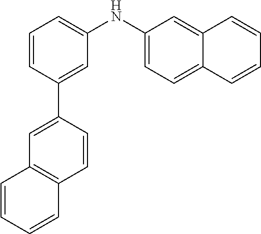 [SMILES: c1cc(Nc2ccc3ccccc3c2)cc(-c2ccc3ccccc3c2)c1] | 48% |
| 610 | 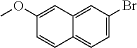 | 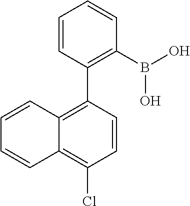 [SMILES: OB(O)c1ccccc1-c1ccc(Cl)c2ccccc12] | 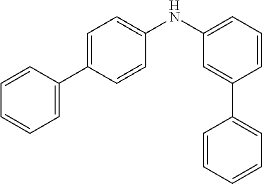 [SMILES: c1ccc(-c2ccc(Nc3cccc(-c4ccccc4)c3)cc2)cc1] | 49% |
| 617 | 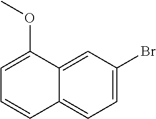 | 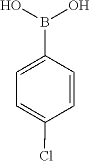 | 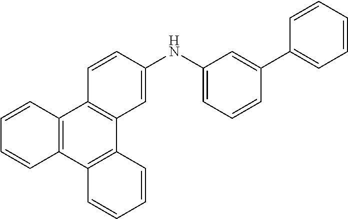 [SMILES: c1ccc(-c2cccc(Nc3ccc4c5ccccc5c5ccccc5c4c3)c2)cc1] | 50% |
| 630 | 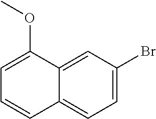 | 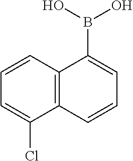 [SMILES: OB(O)c1cccc2c(Cl)cccc12] | 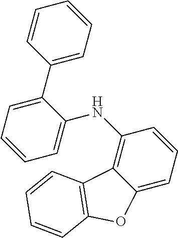 [SMILES: c1ccc(-c2ccccc2Nc2cccc3oc4ccccc4c23)cc1] | 48% |
| 659 | 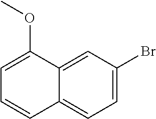 | 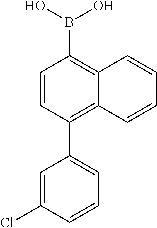 [SMILES: OB(O)c1ccc(-c2cccc(Cl)c2)c2ccccc12] | 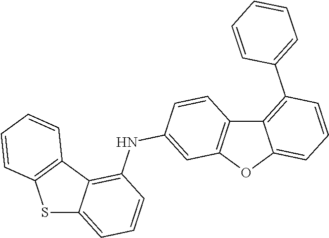 [SMILES: c1ccc(-c2cccc3oc4cc(Nc5cccc6sc7ccccc7c56)ccc4c23)cc1] | 48% |
| 675 | 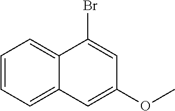 | 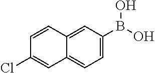 [SMILES: OB(O)c1ccc2cc(Cl)ccc2c1] | 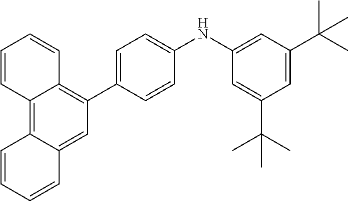 [SMILES: CC(C)(C)c1cc(Nc2ccc(-c3cc4ccccc4c4ccccc34)cc2)cc(C(C)(C)C)c1] | 48% |
| 737 | 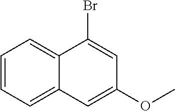 | 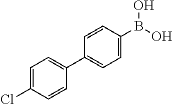 [SMILES: OB(O)c1ccc(-c2ccc(Cl)cc2)cc1] | 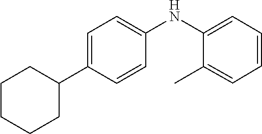 [SMILES: Cc1ccccc1Nc1ccc(C2CCCCC2)cc1] | 56% |
| 740 | 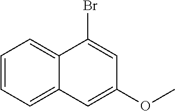 | 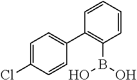 [SMILES: OB(O)c1ccccc1-c1ccc(Cl)cc1] | 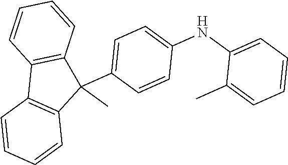 [SMILES: Cc1ccccc1Nc1ccc(C2(C)c3ccccc3-c3ccccc32)cc1] | 49% |
| 752 | 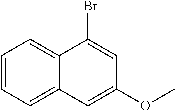 | 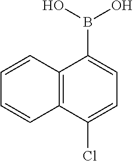 [SMILES: OB(O)c1ccc(Cl)c2ccccc12] | 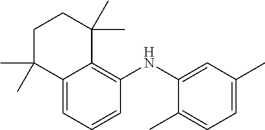 | 52% |
| 772 | 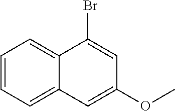 | 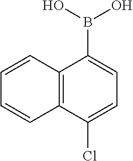 [SMILES: OB(O)c1ccc(Cl)c2ccccc12] | 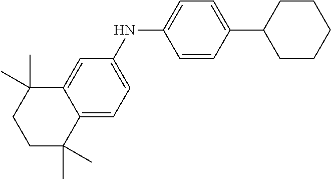 [SMILES: CC1(C)CCC(C)(C)c2cc(Nc3ccc(C4CCCCC4)cc3)ccc21] | 58% |
| 776 | 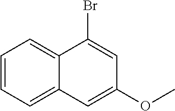 | 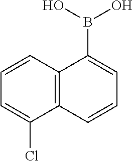 [SMILES: OB(O)c1cccc2c(Cl)cccc12] | 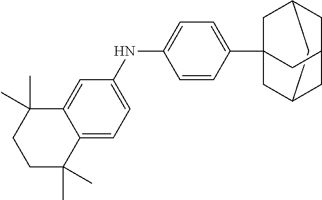 [SMILES: CC1(C)CCC(C)(C)c2cc(Nc3ccc(C45CC6CC(CC(C6)C4)C5)cc3)ccc21] | 51% |
| 791 | 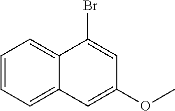 | 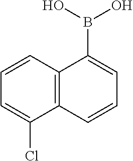 [SMILES: OB(O)c1cccc2c(Cl)cccc12] | 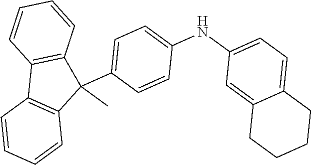 [SMILES: CC1(c2ccc(Nc3ccc4c(c3)CCCC4)cc2)c2ccccc2-c2ccccc21] | 56% |
| 807 | 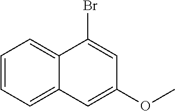 | 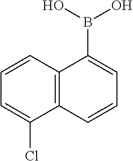 [SMILES: OB(O)c1cccc2c(Cl)cccc12] | 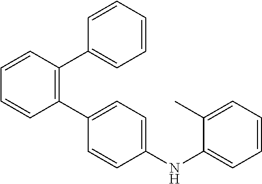 [SMILES: Cc1ccccc1Nc1ccc(-c2ccccc2-c2ccccc2)cc1] | 61% |
| 812 | 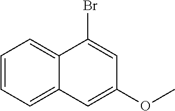 | 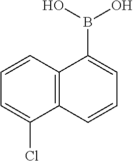 [SMILES: OB(O)c1cccc2c(Cl)cccc12] | 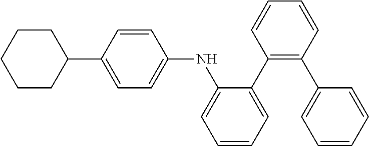 [SMILES: c1ccc(-c2ccccc2-c2ccccc2Nc2ccc(C3CCCCC3)cc2)cc1] | 48% |
| 815 | 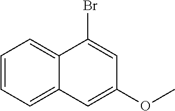 | 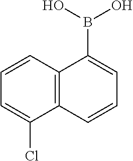 [SMILES: OB(O)c1cccc2c(Cl)cccc12] | 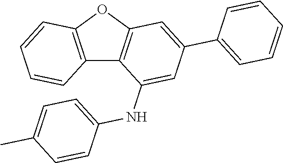 [SMILES: Cc1ccc(Nc2cc(-c3ccccc3)cc3oc4ccccc4c23)cc1] | 52% |
| 835 | 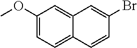 | 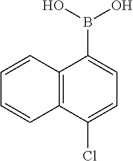 [SMILES: OB(O)c1ccc(Cl)c2ccccc12] | 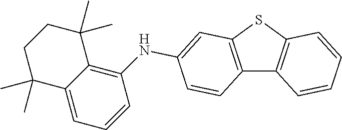 [SMILES: CC1(C)CCC(C)(C)c2c(Nc3ccc4c(c3)sc3ccccc34)cccc21] | 47% |

### [Preparation Example 2] Preparation of Compound 719

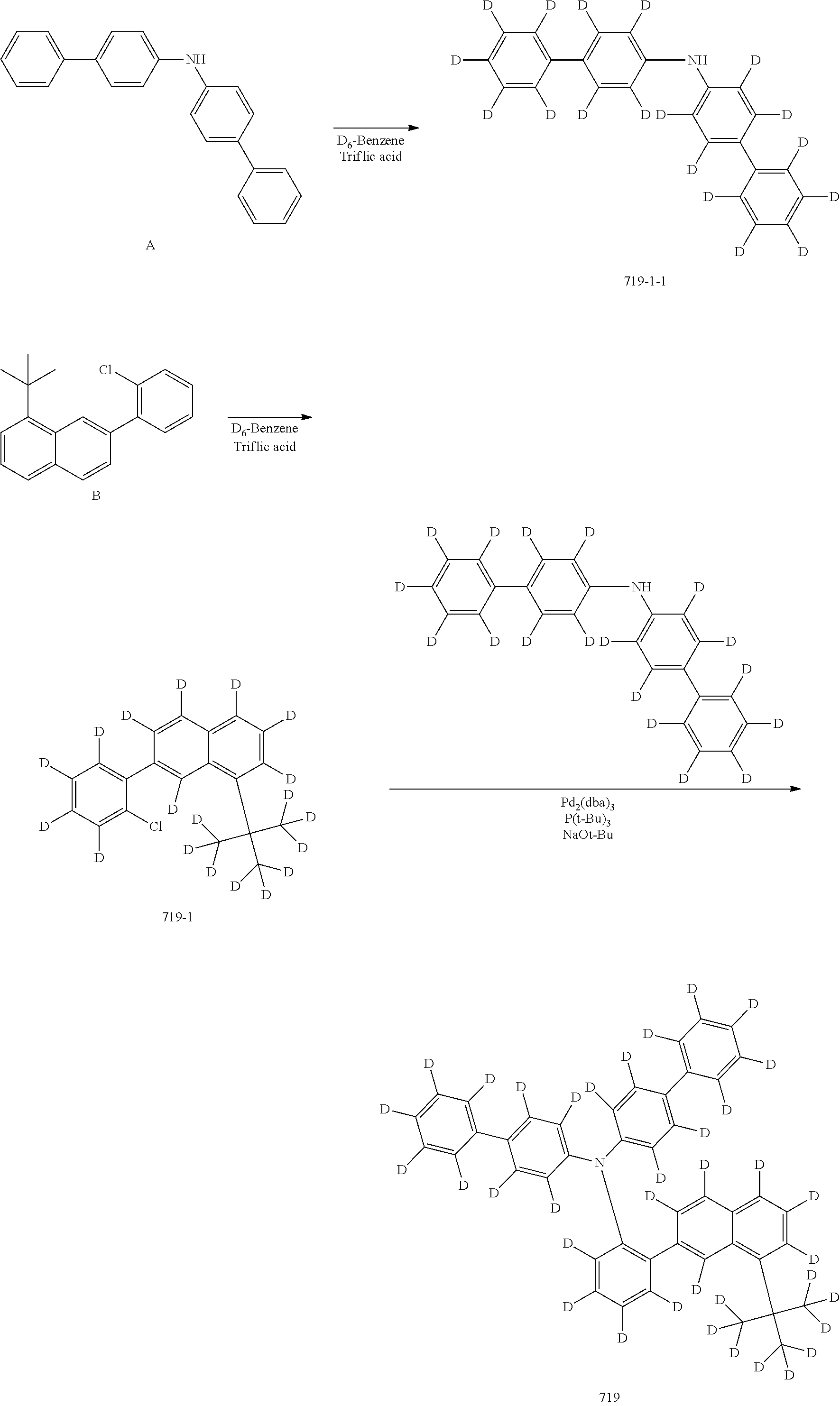

In Preparation Example 2, compound B was synthesized in the same manner as in Preparation Example 1-4. In Preparation Example 2, Compound 719-1-1 and compound 719-1 were synthesized in the same manner, and compound 719 was synthesized in the same manner as compound 1.

### 1) Preparation of Compound 719-1-1

Bis(4-biphenylyl)amine (A) (20 g, 0.062 mol, 1 eq), D₆-benzene (200 mL) and triflic acid (TfOH, 38.3 mL, 0.434 mol, 7.0 eq) were stirred at 60° C. for 2 hours. After terminating reaction by adding water, extraction was performed using MC and water. Then, moisture was 5 removed with MgSO₄. Compound 719-1-1 (18.8 g, 68% yield) was obtained by separating with a silica gel column.

Compounds were synthesized in the same manner, except that intermediate A described in Table 2 was used instead of bis(4-biphenylyl)amine (A), and intermediate B was used instead of 1-(tert-butyl)-7-(2-chlorophenyl)naphthalene (B).

In Table 2, reaction time and TfOH eq respectively refer to the stirring time after addition of triflic acid and the equivalence of triflic acid.

TABLE 2

| Compound | | | Reaction |
|---|---|---|---|
| No. | Intermediate A | Intermediate B | Time |
| 719 | 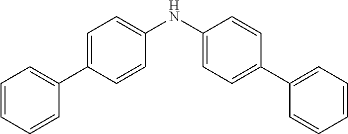 [SMILES: c1ccc(-c2ccc(Nc3ccc(-c4ccccc4)cc3)cc2)cc1] | 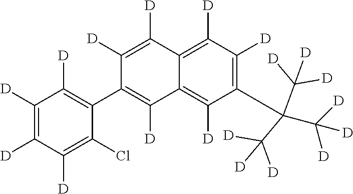 [SMILES: [2H]c1c([2H])c([2H])c(-c2c([2H])c([2H])c3c([2H])c([2H])c(C(C([2H])([2H])[2H])(C([2H])([2H])[2H])C([2H])([2H])[2H])c([2H])c3c2[2H])c(Cl)c1[2H]] | 3 h |
| 717 | 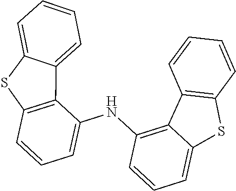 [SMILES: c1ccc2c(c1)sc1cccc(Nc3cccc4sc5ccccc5c34)c12] | 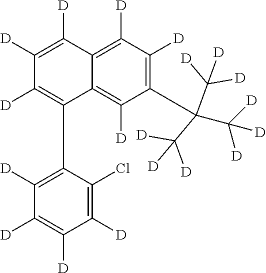 [SMILES: [2H]c1c([2H])c([2H])c(-c2c([2H])c([2H])c([2H])c3c([2H])c([2H])c(C(C([2H])([2H])[2H])(C([2H])([2H])[2H])C([2H])([2H])[2H])c([2H])c23)c(Cl)c1[2H]] | 5 h |
| 720 | 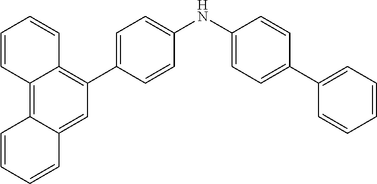 [SMILES: c1ccc(-c2ccc(Nc3ccc(-c4cc5ccccc5c5ccccc45)cc3)cc2)cc1] | 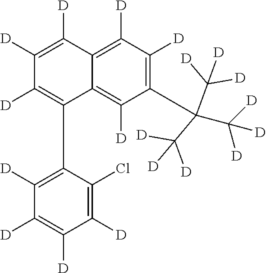 [SMILES: [2H]c1c([2H])c([2H])c(-c2c([2H])c([2H])c([2H])c3c([2H])c([2H])c(C(C([2H])([2H])[2H])(C([2H])([2H])[2H])C([2H])([2H])[2H])c([2H])c23)c(Cl)c1[2H]] | 9 h |

| Compound | TfOH | |
|---|---|---|
| No. | equivalent | Yiled |
| 719 | 5 eq | 68% |
| 717 | 7 eq | 69% |
| 720 | 7 eq | 68% |

Compounds were prepared in the same manner as in the preparation examples, and the result of confirming synthesis is shown in Table 3 and Table 4. Table 3 shows ¹H NMR (CDCl₃, 400 MHz) measurement data, and Table 4 shows FD-MS (field desorption mass spectrometry) measurement data.

TABLE 3

| Compound | ¹H NMR (CDCl₃, 400 MHz) |
|---|---|
| 1 | δ = 8.22 (1H, d), 8.10-8.06 (2H, m), 7.75-7.34 (25H, m), 1.48 (9H, s) |
| 36 | δ = 9.08 (1H, d), 9.00 (2H, d), 8.84 (1H, d), 8.27-8.22 (2H, m), 8.10-8.05 (3H, m), |
| | 7.90 (1H, d), 7.75-7.34 (28H, m), 1.48 (9H, s) |
| 54 | δ = 8.61 (1H, d), 8.47-8.45 (2H, d), 8.22-8.06 (5H, m), 7.96-7.93 (3H, m), 7.77- |
| | 7.27 (19H, m), 7.18 (2H, d), 1.48 (9H, s) |
| 62 | δ = 9.08 (1H, d), 8.84 (1H, d), 8.27 (1H, d), 8.14-7.89 (6H, m), 7.70-7.52 (11H, |
| | m), 7.37-7.27 (7H, m), 7.18 (2H, d), 6.97 (1H, d), 1.48 (9H, s) |
| 96 | δ = 9.60 (1H, d), 9.27 (1H, s), 8.89 (2H, d), 8.37-8.30 (5H, m), 8.14 (2H, d), 7.89 |
| | (1H, s), 7.70-7.52 (14H, m), 7.37-7.24 (7H, m), 7.08-7.00 (3H, m), 1.48 (9H, s) |
| 108 | δ = 9.00 (2H, d), 8.55 (1H, d), 8.45 (1H, d), 8.32 (1H, d), 8.14 (2H, d), 7.89 (1H, |
| | d) 7.70-7.68 (2H, m), 7.61-7.49 (6H, m), 7.39-7.00 (14H, m), 1.48 (9H, s) |
| 120 | δ = 9.08 (1H, d), 8.84-8.80 (2H, m), 8.48 (1H, d), 8.27 (1H, d), 8.05-7.90 (3H, m), |
| | 7.78-7.37 (17H, m), 7.27 (2H, d), 7.18-7.17 (4H, m), 6.85 (1H, d), 1.48 (9H, s) |
| 130 | δ = 8.80 (1H, d), 8.48 (1H, d), 8.02-7.96 (3H, m), 7.79-7.75 (5H, m), 7.60-7.37 |
| | (15H, m), 7.27-7.17 (10H, m), 6.85 (1H, d), 1.48 (9H, s) |
| 142 | δ = 8.80 (1H, d), 8.48 (1H, d), 8.09-7.94 (7H, m), 7.80-7.73 (3H, m), 7.63-7.50 |
| | (10H, m), 7.39-7.31 (6H, m), 7.18 (2H, m), 6.91-6.85 (2H, d), 1.48 (9H, s) |
| 171 | δ = 8.58 (1H, d), 8.04-7.98 (2H, m), 7.84-7.75 (5H, m), 7.59-7.41 (18H, m), 7.25 |
| | (4H, m), 7.17-7.11 (2H, m), 6.99 (1H, d), 1.48 (9H, s) |
| 178 | δ = 8.95 (1H, d), 8.58 (1H, d), 8.27 (1H, d), 8.09-7.98 (5H, m), 7.78-7.71 (3H, m), |
| | 7.61-7.37 (17H, m), 7.11 (1H, s), 6.99 (1H, d), 1.48 (9H, s) |
| 223 | δ = 8.89 (2H, d), 8.58 (1H, d), 8.45 (1H, d), 8.37 (2H, d), 8.10-7.93 (6H, m), 7.85 |
| | (1H, d), 7.77-7.70 (3H, m), 7.59-7.37 (11H, d), 7.14-7.08 (4H, m), 6.99 (1H, d), |
| | 1.48 (9H, s) |
| 234 | δ = 8.95 (1H, d), 8.37 (1H, d), 8.27-8.22 (3H, m), 8.06 (1H, d), 7.98 (1H, d), 7.74 |
| | (1H, d), 7.73 (2H, s), 7.61-7.49 (9H, m), 7.39-7.31 (5H, m), 7.00-6.97 (2H, m), |
| | 1.48 (9H, s), 1.32 (18H, s) |
| 249 | δ = 8.37 (1H, d), 8.25 (1H, s), 8.22 (1H, s), 8.08-7.98 (6H, d), 7.74-7.73 (2H, m), |
| | 7.61-7.51 (11H, m), 7.39-7.31 (8H, m), 7.00-6.97 (2H, m), 1.48 (9H, s) |
| 261 | δ = 8.98 (1H, d), 8.84 (1H, d), 8.61 (1H, d), 8.47 (1H, d), 8.37 (1H, d), 8.25 (1H, |
| | s), 8.16-8.06 (3H, m), 7.90 (1H, d), 7.77-7.56 (8H, m), 7.49-7.45 (2H, m), 7.25- |
| | 7.24 (6H, m), 7.08-7.00 (4H, m), 1.48 (9H, s) |
| 288 | δ = 9.08 (1H, d), 8.85-8.84 (2H, m), 8.27 (1H, d), 8.08 (1H, d), 8.05 (1H, s), 7.91- |
| | 7.90 (2H, m), 7.75-7.27 (19H, m), 7.18-7.17 (4H, m), 7.09 (1H, d), 1.41 (9H, s) |
| 293 | δ = 8.61 (1H, d), 8.51-8.47 (2H, m), 8.16-8.05 (3H, m), 7.92 (1H, d), 7.79-7.75 |
| | (8H, m), 7.56-7.37 (18H, m), 6.91 (1H, d), 1.48 (9H, s) |
| 303 | δ = 8.85 (1H, d), 8.08 (1H, d), 7.98-7.91 (2H, m), 7.75 (2H, d), 7.55-7.17 (22H, |
| | m), 7.09 (1H, d), 6.91 (1H, d), 1.41 (9H, s) |
| 305 | δ = 8.51-8.45 (2H, m), 8.09-8.05 (2H, m), 7.94 (1H, s), 7.93 (1H, d), 7.75-7.73 |
| | (4H, m), 7.61-7.37 (19H, m), 6.91 (1H, d), 1.48 (9H, s) |
| 325 | δ = 8.61 (1H, d), 8.51-8.47 (2H, m), 8.16-7.94 (8H, m), 7.79-7.73 (5H, m), 7.61- |
| | 7.25 (17H, m), 7.14 (1H, d), 6.91 (2H, d), 1.48 (9H, s) |
| 331 | δ = 8.95 (1H, d), 8.85 (1H, d), 8.27 (1H, d), 8.08-7.91 (6H, m), 7.82-7.79 (4H, m), |
| | 7.69 (1H, d), 7.61-7.25 (17H, m), 7.09 (1H, d), 6.91 (2H, d), 1.41 (9H, s) |
| 358 | δ = 8.55 (1H, d), 8.32 (1H, d), 8.17 (1H, s), 8.11 (1H, d), 7.98-7.97 (2H, m), 7.85 |
| | (1H, s), 7.70 (1H, dd), 7.55-7.41 (10H, m), 7.27-7.17 (9H, m), 7.08-7.00 (3H, m), |
| | 1.41 (9H, s) |
| 380 | δ = 9.00 (2H, d), 8.17 (1H, s), 7.97 (2H, d), 7.85-7.75 (5H, m), 7.65-7.39 (25H, |
| | m), 7.27 (1H, s), 7.18 (2H, m), 1.41 (9H, s) |
| 383 | δ = 8.89 (2H, d), 8.37 (2H, d), 8.17 (1H, s), 7.98 (2H, m), 7.85-7.70 (12H, m), |
| | 7.57-7.37 (15H, m), 7.27 (2H, s), 7.18-7.17 (4H, m), 1.41 (9H, s) |
| 396 | δ = 8.95 (1H, d), 8.83 (1H, d), 8.45 (1H, d), 8.27 (1H, d), 8.11 (2H, d), 7.93 (1H, |
| | d), 7.89 (1H, s), 7.79 (2H, d), 7.72 (1H, s), 7.56-7.37 (16H, m), 7.27 (1H, d), 1.38 |
| | (9H, s) |
| 403 | δ = 8.95 (1H, d), 8.83 (1H, d), 8.45 (1H, d), 8.27 (1H, d), 8.11 (2H, d), 7.93 (1H, |
| | d), 7.89 (1H, s), 7.72 (1H, s), 7.56-7.27 (18H, m), 1.38 (9H, s), 1.33 (9H, s) |
| 426 | δ = 8.95 (1H, d), 8.83 (1H, d), 8.27-8.10 (4H, m), 7.89 (1H, s), 7.81 (1H, d), 7.72 |
| | (1H, s), 7.63-7.37 (9H, m), 7.27-7.24 (7H, m), 7.08-7.00 (3H, m), 1.38 (9H, s) |
| 455 | δ = 8.98 (1H, d), 8.84 (1H, d), 8.40 (1H, d), 8.11-8.10 (2H, m), 8.00-7.90 (4H, m), |
| | 7.68-7.37 (12H, m), 7.27 (1H, s), 7.18-7.06 (6H, m), 1.49 (9H, s) |
| 480 | δ = 8.45-8.40 (2H, m), 8.01-7.93 (6H, m), 7.84 (1H, d), 7.75-7.40 (16H, m), 7.32 |
| | (1H, s), 7.27 (1H, s), 7.18-7.06 (5H, m), 1.49 (9H, s) |
| 489 | δ = 8.95 (1H, s), 8.45-8.40 (2H, m), 8.27 (1H, d), 8.10 (1H, d), 8.01-7.93 (8H, m), |
| | 7.79-7.37(21H, m), 7.14 (1H, d), 7.06 (1H, d), 1.49 (9H, s) |
| 508 | δ = 8.61 (1H, d), 8.47 (1H, d), 8.16 (1H, d), 7.89 (1H, d), 7.81-7.77 (4H, m), 7.66 |
| | (1H, s), 7.56-7.37 (11H, m), 7.26-7.24 (3H, m), 7.08-7.00 (3H, m), 1.49 (9H, s) |
| 518 | δ = 8.61 (1H, d), 8.47 (1H, d), 8.16 (1H, d), 7.89 (1H, d), 7.81-7.77 (4H, m), 7.66- |
| | 7.41 (16H, m), 7.26-7.24 (3H, m), 7.08-7.00 (3H, m), 1.49 (9H, s) |
| 559 | δ = 8.95-8.89 (3H, m), 8.50-8.45 (2H, m), 8.37 (2H, d), 8.20 (1H, d), 8.10 (2H, |
| | d), 7.95-7.66 (9H, m), 7.56-7.37 (9H, m), 7.27 (1H, s), 7.26 (2H, d), 7.18-7.14 |
| | (3H, m), 1.49 (9H, s) |
| 573 | δ = 8.61 (1H, d), 8.47-8.45 (2H, m), 8.10 (1H, d), 7.98-7.85 (6H, m), 7.77 (1H, |
| | dd), 7.64-7.28 (14H, m), 6.97 (1H, d), 1.49 (9H, s) |
| 590 | δ = 8.09-8.06 (2H, m), 7.99-7.87 (4H, m), 7.78-7.71 (3H, m), 7.63-7.38 (15H, m), |
| | 7.28 (1H, d), 7.27 (2H, s), 7.18-7.11 (5H, m), 1.49 (9H, s) |
| 610 | δ = 8.95 (1H, d), 8.27 (1H, d), 7.96-7.87 (4H, m), 7.75 (4H, d), 7.60-7.37 (20H, |
| | m), 7.28 (1H, d), 7.27 (1H, s), 7.18 (2H, d), 1.49 (9H, s) |
| 617 | δ = 9.08 (1H, d), 8.71 (1H, s), 8.38-8.33 (2H, m), 8.07-7.93 (4H, m), 7.70-7.37 |
| | (18H, m), 7.27 (1H, s), 7.18 (2H, d), 7.00 (1H, d), 1.49 (9H, s) |
| 630 | δ = 8.61 (1H, d), 8.47 (1H, d), 8.38 (1H, d), 8.16-8.10 (2H, m), 8.02-7.93 (4H, m), |
| | 7.77 (1H, dd), 7.67 (1H, dd), 7.54-7.43 (2H, m), 7.45-7.25 (10H, m), 7.14-7.08 |
| | (3H, m), 7.00 (1H, d), 6.91 (1H, d), 1.49 (9H, s) |
| 659 | δ = 9.00 (2H, d), 8.45-8.38 (2H, m), 8.03-7.93 (5H, m), 7.82-7.79 (4H, m), 7.69- |
| | 7.41 (16H, m), 7.27 (1H, s), 7.17 (2H, d), 7.00 (1H, d), 6.91 (1H, d), 1.49 (9H, s) |
| 675 | δ = 9.08 (1H, d), 8.84 (1H, d), 8.27 (1H, d), 8.14 (2H, d), 8.05 (1H, s), 7.90-7.84 |
| | (3H, m), 7.70-7.52 (9H, m), 7.40-7.32 (5H, m), 7.20-7.11 (5H, m), 1.48 (9H, s), |
| | 1.41 (18H, s) |
| 717 | Fully D-substituted |
| 719 | Fully D-substituted |
| 720 | Fully D-substituted |
| 737 | δ = 8.14 (2H, d), 7.89 (1H, s), 7.55-7.52 (3H, m), 7.38-7.33 (3H, m), 7.25-7.06 |
| | (11H, m), 6.91 (1H, t), 2.72 (1H, t), 2.12 (3H, s), 1.85-1.42 (19H, m) |
| 740 | δ = 8.14 (2H, d), 7.96-7.90 (4H, m), 7.68 (1H, s), 7.60-7.52 (7H, m), 7.38-7.13 |
| | (14H, m), 6.91 (1H, t), 2.28 (3H, s), 2.12 (3H, s), 1.48 (9H, s) |
| 752 | δ = 8.95 (1H, d), 8.27 (1H, d), 8.14-8.13 (2H, d), 7.89 (1H, s), 7.68 (1H, s), 7.54- |
| | 7.33 (5H, m), 7.20 (1H, s), 6.98 (1H, d), 6.88 (1H, t), 6.72 (1H, d), 6.58 (1H, d), |
| | 6.45 (1H, d), 2.27 (3H, s), 2.12 (3H, s), 1.48 (9H, s), 0.91 (12H, s) |
| 772 | δ = 8.95 (1H, d), 8.27 (1H, d), 8.14-8.13 (2H, d), 7.89 (1H, s), 7.68-7.61 (2H, m), |
| | 7.54-7.48 (3H, m), 7.37-7.33 (2H, m), 7.18 (2H, d), 7.06 (2H, m), 6.88-6.79 (2H, |
| | d), 6.45 (1H, d), 2.72 (1H, t), 1.85-1.43(23H, m), 0.91 (12H, s) |
| 776 | δ = 8.61-8.47 (2H, d), 8.14-8.12 (3H, m), 7.89 (1H, s), 7.77 (1H, m), 7.56-7.46 |
| | (3H, m), 7.33 (1H, m), 7.10 (4H, d), 6.88-6.80 (2H, m), 6.45 (1H, d), 2.02-1.98 |
| | (9H, m), 1.72 (6H, t), 1.48 (13H, s), 0.91 (12H, s) |
| 791 | δ = 8.61 (1H, d), 8.47 (1H, d), 8.14-8.13 (2H, d), 7.90-7.89 (3H, m), 7.77-7.68 |
| | (1H, d), 7.56-7.28 (10H, m), 7.17-7.14 (4H, m), 6.83-6.79 (2H, d), 6.45 (1H, d), |
| | 2.70 (4H, t), 2.28 (3H, s), 1.74 (4H, t), 1.48 (9H, s) |
| 807 | δ = 8.61-8.47 (2H, d), 8.16-8.12 (3H, m), 7.96 (2H, m), 7.79-7.77 (3H, m), 7.68- |
| | 7.33 (14H, m), 7.19-7.12 (3H, m), 6.91 (1H, m), 2.12 (3H, s), 1.72 (6H, t), 1.48 |
| | (9H, s) |
| 812 | δ = 8.61-8.47 (2H, d), 8.16-8.12 (4H, m), 7.96 (2H, m), 7.89 (1H, s), 7.79-7.77 |
| | (3H, m), 7.60-7.33 (11H, m), 7.18-7.06 (5H, m), 2.72 (1H, m), 1.85-1.43 (19H, s) |
| 815 | δ = 8.61-8.47 (2H, d), 8.20-8.13 (4H, m), 7.98 (1H, d), 7.89 (1H, s), 7.77-7.68 |
| | (4H, m), 7.56-7.33 (11H, m), 7.15-7.13 (4H, m), 2.32 (3H, s), 1.48 (9H, s) |
| 835 | δ = 8.95 (1H, d), 8.45-8.27 (2H, m), 8.01 (1H, d), 7.93-7.87 (3H, s), 7.58-7.43 |
| | (11H, m), 6.88 (1H, m), 6.58-6.28 (2H, m), 1.49-1.48 (13H, s), 0.91 (12H, s) |

TABLE 4

| Compound | FD-MS | Compound | FD-MS |
|---|---|---|---|
| 1 | m/z = 529.29 | 36 | m/z = 805.37 |
| | (C₄₄H₃₇N = 529.87) | | (C₆₂H₄₇N = 807.01) |
| 54 | m/z = 735.30 | 62 | m/z = 693.30 |
| | (C₅₄H₄₁NS = 735.74) | | (C₅₂H₃₉NO = 693.88) |
| 96 | m/z = 779.36 | 108 | m/z = 735.30 |
| | (C₆₀H₄₅N = 779.98) | | (C₅₄H₄₁NS = 735.86) |
| 120 | m/z = 679.32 | 130 | m/z = 731.36 |
| | (C₅₂H₄₁N = 680.02) | | (C₅₆H₄₅N = 732.17) |
| 142 | m/z = 719.32 | 171 | m/z = 705.34 |
| | (C₅₄H₄₁NO = 720.04) | | (C₅₄H₄₃N = 705.85) |
| 178 | m/z = 653.31 | 223 | m/z = 735.30 |
| | (C₅₀H₃₉N = 653.91) | | (C₅₄H₄₁NS = 736.16) |
| 234 | m/z = 755.41 | 249 | m/z = 759.31 |
| | (C₅₆H₅₃NO = 756.24) | | (C₅₆H₄₁NO₂ = 760.18) |
| 261 | m/z = 653.31 | 288 | m/z = 679.32 |
| | (C₅₀H₃₉N = 654.29) | | (C₅₂H₄₁N = 679.95) |
| 293 | m/z = 705.34 | 303 | m/z = 669.30 |
| | (C₅₄H₄₃N = 706.09) | | (C₅₀H₃₉NO = 670.21) |
| 305 | m/z = 685.28 | 325 | m/z = 795.35 |
| | (C₅₀H₃₉NS = 685.99) | | (C₆₀H₄₅NO = 796.14) |
| 331 | m/z = 809.33 | 358 | m/z = 685.28 |
| | (C₆₀H₄₃NO₂ = 809.89) | | (C₅₀H₃₉NS = 685.75) |
| 380 | m/z = 781.37 | 383 | m/z = 871.38 |
| | (C₆₀H₄₇N = 782.14) | | (C₆₆H₄₉NO = 872.06) |
| 396 | m/z = 659.26 | 403 | m/z = 715.33 |
| | (C₄₈H₃₇NS = 659.88) | | (C₅₂H₄₅NS = 716.21) |
| 426 | m/z = 603.29 | 455 | m/z = 603.29 |
| | (C₄₆H₃₇N = 604.04) | | (C₄₆H₃₇N = 604.07) |
| 480 | m/z = 735.30 | 489 | m/z = 811.33 |
| | (C₅₄H₄₁NS = 736.25) | | (C₆₀H₄₅NS = 812.11) |
| 508 | m/z = 557.28 | 518 | m/z = 629.31 |
| | (C₄₂H₃₅N = 558.84) | | (C₄₈H₃₉N = 629.86) |
| 559 | m/z = 785.31 | 573 | m/z = 673.24 |
| | (C₅₈H₄₃NS = 786.26) | | (C₄₈H₃₅NOS = 673.77) |
| 590 | m/z = 679.32 | 610 | m/z = 705.34 |
| | (C₅₂H₄₁N = 679.93) | | (C₅₄H₄₃N = 706.21) |
| 617 | m/z = 653.31 | 630 | m/z = 643.29 |
| | (C₅₀H₃₉N = 654.14) | | (C₄₈H₃₇NO = 643.88) |
| 659 | m/z = 825.31 | 675 | m/z = 765.43 |
| | (C₆₀H₄₃NOS = 826.05) | | (C₅₈H₅₅N = 766.14) |
| 717 | m/z = 672.41 | 719 | m/z = 616.52 |
| | (C₄₄D₃₃NS₂ = 673.26) | | (C₄₄D₃₇N = 617.39) |
| 720 | m/z = 724.61 | 737 | m/z = 599.36 |
| | (C₅₂D₄₃N = 725.42) | | (C₄₅H₄₅N = 600.12) |
| 740 | m/z = 695.36 | 752 | m/z = 615.39 |
| | (C₅₃H₄₅N = 696.17) | | (C₄₆H₄₉N = 616.21) |
| 772 | m/z = 669.43 | 776 | m/z = 721.46 |
| | (C₅₀H₅₅N = 670.14) | | (C₅₄H₅₉N = 722.10) |
| 791 | m/z = 709.37 | 807 | m/z = 643.32 |
| | (C₅₄H₄₇N = 710.05) | | (C₄₉H₄₁N = 643.90) |
| 812 | m/z = 711.39 | 815 | m/z = 657.30 |
| | (C₅₄H₄₉N = 712.04) | | (C₄₉H₃₉NO = 658.11) |
| 835 | m/z = 693.34 | | |
| | (C₅₀H₄₇NS = 694.19) | | |

### TEST EXAMPLES

### Test Example 1

### 1) Manufacturing of Organic Light-Emitting Device

A transparent electrode ITO thin film obtained from an OLED glass (Samsung Corning) was ultrasonically cleansed sequentially using trichloroethylene, acetone, ethanol and distilled water for 5 minutes respectively, and then stored in isopropanol until use. Then, the ITO substrate was mounted on the substrate folder of a vacuum deposition system, and 4,4′,4″-tris(N,N-(2-naphthyl)-phenylamino)triphenylamine (2-TNATA) was added to a cell of the vacuum deposition system.

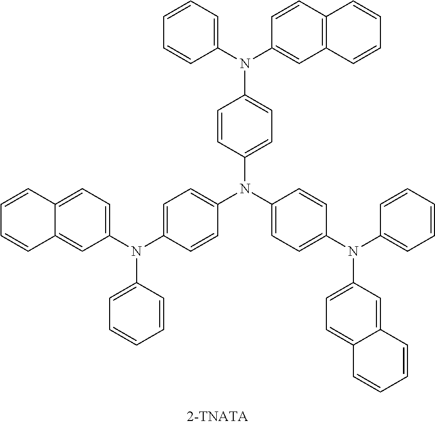 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Then, after evacuating the chamber until the vacuum level reached 10⁻⁶ torr, 2-TNATA was evaporated by applying a current to the cell, and a hole injection layer with a thickness of 600 Å was deposited on the ITO substrate. After adding N,N′-bis(α-naphthyl)-N,N′-diphenyl-4,4′-diamine (NPB) to another cell of the vacuum deposition system, NPB was evaporated by applying a current to the cell, and a hole transport layer with a thickness of 300 Å was deposited on the hole injection layer.

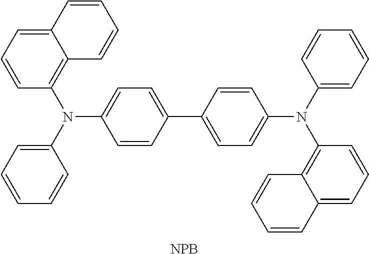 [SMILES: c1ccc(N(c2ccc(-c3ccc(N(c4ccccc4)c4cccc5ccccc45)cc3)cc2)c2cccc3ccccc23)cc1 ; "NPB"]

After the hole injection layer and the hole transport layer were formed, blue light-emitting materials with the following structures were deposited thereon as a light-emitting layer. Specifically, in one cell of the vacuum deposition system, a blue light-emitting host material H1 was vacuum-deposited to a thickness of 200 Å, and 5% of a blue light-emitting dopant material D1 was vacuum-deposited thereon based on the host material.

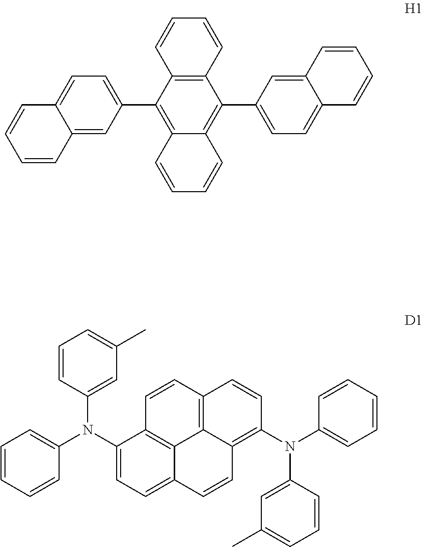

Subsequently, a compound of Structural Formula E1 was deposited to a thickness of 300 Å as an electron transport layer.

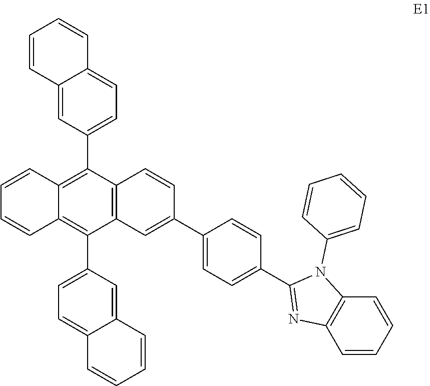 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

After depositing lithium fluoride (LiF) to a thickness of 10 Å as an electron injection layer, an OLED device was manufactured by depositing an Al negative electrode to a thickness of 1,000 Å. All the organic compounds required for the manufacturing of the OLED device were purified by vacuum sublimation at 10⁻⁸ to 10⁻⁶ torr. Organic light-emitting devices were manufactured using in the same manner as in Test Example 1, except that the compounds described in Table 5 were used instead of NPB during the formation of the hole transport layer. The electroluminescence (EL) characteristics of the manufactured organic light-emitting devices were measured using Mcscience's M7000, and T₉₅ at a reference luminance of 700 cd/m² was measured from the measurement result using a Mcscience's lifetime test system (M6000). The result of measuring the operation voltage, luminous efficiency, color coordinates (CIE) and lifetime of the blue organic light-emitting devices manufactured according to the present disclosure is shown in Table 5.

TABLE 5

| | | Operation | Luminous | | |
|---|---|---|---|---|---|
| | | voltage | efficiency | CIE | Lifetime |
| No. | Compound | (V) | (cd/A) | (x, y) | (T₉₅) |
| Example 1 | 001 | 4.81 | 6.80 | (0.133, 0.100) | 85 |
| Example 2 | 036 | 4.85 | 6.79 | (0.134, 0.101) | 87 |
| Example 3 | 054 | 4.84 | 6.81 | (0.133, 0.100) | 90 |
| Example 4 | 062 | 4.83 | 6.85 | (0.133, 0.100) | 88 |
| Example 5 | 096 | 4.84 | 6.78 | (0.133, 0.101) | 86 |
| Example 6 | 108 | 4.75 | 6.76 | (0.134, 0.101) | 90 |
| Example 7 | 120 | 4.78 | 6.88 | (0.133, 0.100) | 89 |
| Example 8 | 130 | 4.82 | 6.84 | (0.133, 0.101) | 92 |
| Example 9 | 142 | 4.81 | 6.81 | (0.134, 0.101) | 89 |
| Example 10 | 171 | 4.80 | 6.80 | (0.134, 0.100) | 88 |
| Example 11 | 178 | 4.83 | 6.88 | (0.133, 0.100) | 90 |
| Example 12 | 223 | 4.83 | 6.83 | (0.134, 0.101) | 86 |
| Example 13 | 234 | 4.82 | 6.87 | (0.133, 0.100) | 89 |
| Example 14 | 249 | 4.77 | 6.78 | (0.133, 0.100) | 87 |
| Example 15 | 261 | 4.85 | 6.89 | (0.133, 0.101) | 89 |
| Example 16 | 288 | 4.83 | 6.84 | (0.134, 0.100) | 86 |
| Example 17 | 293 | 4.84 | 6.80 | (0.134, 0.100) | 88 |
| Example 18 | 303 | 4.79 | 6.72 | (0.133, 0.101) | 86 |
| Example 19 | 305 | 4.80 | 6.86 | (0.134, 0.100) | 86 |
| Example 20 | 325 | 4.84 | 6.81 | (0.133, 0.101) | 85 |
| Example 21 | 331 | 4.85 | 6.87 | (0.133, 0.101) | 88 |
| Example 22 | 358 | 4.84 | 6.71 | (0.134, 0.101) | 85 |
| Example 23 | 380 | 4.81 | 6.90 | (0.133, 0.101) | 89 |
| Example 24 | 383 | 4.78 | 6.87 | (0.134, 0.100) | 86 |
| Example 25 | 396 | 4.83 | 6.84 | (0.133, 0.100) | 88 |
| Example 26 | 403 | 4.84 | 6.81 | (0.133, 0.100) | 91 |
| Example 27 | 426 | 4.81 | 6.84 | (0.133, 0.100) | 87 |
| Example 28 | 455 | 4.80 | 6.88 | (0.134, 0.100) | 85 |
| Example 29 | 480 | 4.82 | 6.87 | (0.134, 0.101) | 86 |
| Example 30 | 489 | 4.81 | 6.90 | (0.133, 0.100) | 89 |
| Example 31 | 508 | 4.78 | 6.86 | (0.133, 0.101) | 87 |
| Example 32 | 518 | 4.85 | 6.87 | (0.134, 0.100) | 89 |
| Example 33 | 559 | 4.82 | 6.86 | (0.133, 0.100) | 88 |
| Example 34 | 573 | 4.76 | 6.84 | (0.133, 0.101) | 90 |
| Example 35 | 590 | 4.80 | 6.86 | (0.133, 0.100) | 91 |
| Example 36 | 610 | 4.84 | 6.74 | (0.134, 0.100) | 86 |
| Example 37 | 617 | 4.82 | 6.91 | (0.134, 0.100) | 89 |
| Example 38 | 630 | 4.82 | 6.87 | (0.133, 0.101) | 87 |
| Example 39 | 659 | 4.83 | 6.93 | (0.133, 0.101) | 89 |
| Example 40 | 675 | 4.85 | 6.89 | (0.134, 0.101) | 90 |
| Example 41 | 717 | 4.80 | 6.76 | (0.134, 0.100) | 92 |
| Example 42 | 719 | 4.85 | 6.71 | (0.134, 0.101) | 94 |
| Example 43 | 720 | 4.84 | 6.73 | (0.134, 0.100) | 93 |
| Example 44 | 737 | 4.80 | 6.83 | (0.132, 0.99) | 95 |
| Example 45 | 740 | 4.81 | 6.91 | (0.134, 0.101) | 91 |
| Example 46 | 752 | 4.75 | 6.88 | (0.134, 0.100) | 92 |
| Example 47 | 772 | 4.82 | 6.84 | (0.133, 0.98) | 87 |
| Example 48 | 776 | 4.80 | 6.80 | (0.134, 0.101) | 88 |
| Example 49 | 791 | 4.84 | 6.85 | (0.134, 0.100) | 89 |
| Example 50 | 807 | 4.77 | 6.94 | (0.133, 0.100) | 97 |
| Example 51 | 812 | 4.78 | 6.91 | (0.134, 0.100) | 94 |
| Example 52 | 815 | 4.81 | 6.92 | (0.133, 0.100) | 95 |
| Example 53 | 835 | 4.85 | 6.87 | (0.134, 0.101) | 90 |
| Comparative | NPB | 5.54 | 6.05 | (0.134, 0.100) | 61 |
| Example 1 | | | | | |
| Comparative | Comparative | 5.29 | 6.15 | (0.133, 0.100) | 65 |
| Example 2 | compound A | | | | |
| Comparative | Comparative | 5.30 | 6.19 | (0.134, 0.101) | 61 |
| Example 3 | compound B | | | | |
| Comparative | Comparative | 5.39 | 6.42 | (0.133, 0.100) | 67 |
| Example 4 | compound C | | | | |
| Comparative | Comparative | 5.36 | 6.23 | (0.133, 0.101) | 66 |
| Example 5 | compound D | | | | |
| Comparative | Comparative | 5.28 | 6.26 | (0.133, 0.101) | 62 |
| Example 6 | compound E | | | | |
| Comparative | Comparative | 5.37 | 6.21 | (0.133, 0.100) | 61 |
| Example 7 | compound F | | | | |
| Comparative | Comparative | 5.35 | 6.28 | (0.132, 0.101) | 66 |
| Example 8 | compound G | | | | |

The structures of comparative compounds A to G are as follows.

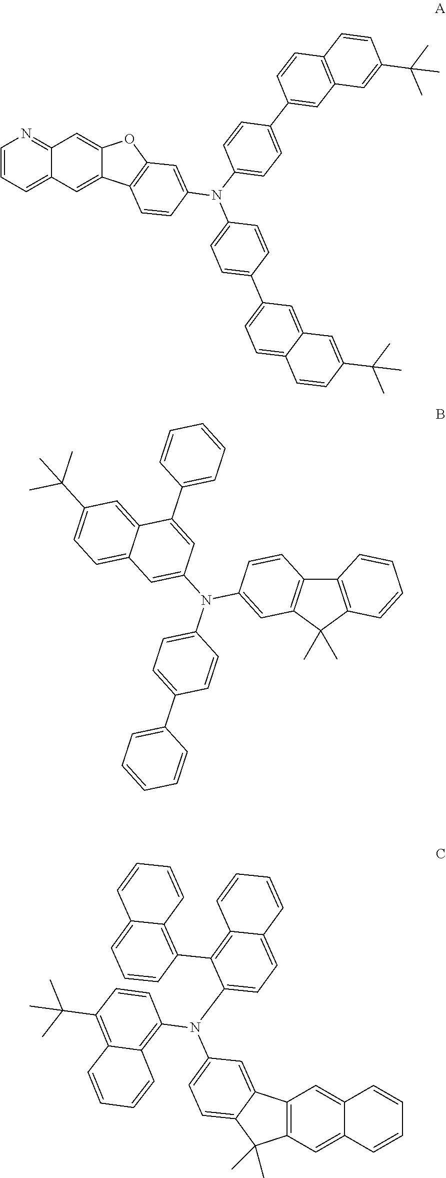

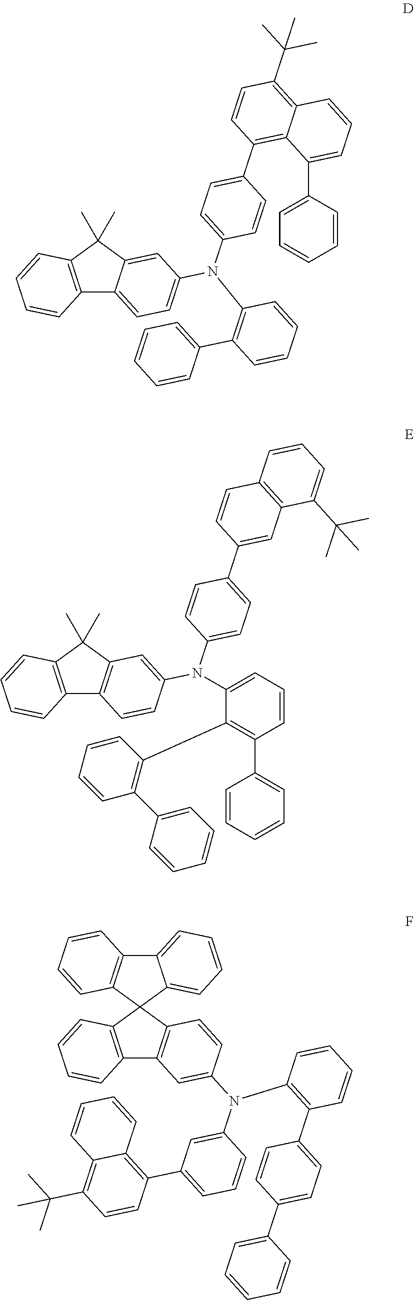

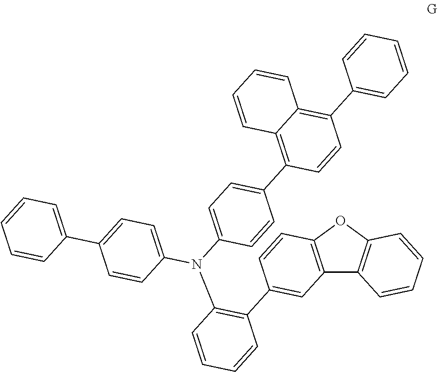 [SMILES: c1ccc(-c2ccc(N(c3ccc(-c4ccc(-c5ccccc5)c5ccccc45)cc3)c3ccccc3-c3ccc4oc5ccccc5c4c3)cc2)cc1]

As can be seen from Table 5, the blue organic light-emitting device using the hole transport layer material of the present disclosure showed a lower operation voltage and significantly improved luminous efficiency and lifetime as compared to Comparative Examples 1 to 8.

The comparative compound A of Comparative Example 2, which contains a tetracyclic heteroaryl group, is different from the compound of the present disclosure containing a tricyclic heteroaryl group. The device presented in the present disclosure provides an appropriate HOMO energy level required for the device due to the characteristics of the fused structure derived by the tricyclic heteroaryl group, rather than the tetracyclic group. As a result, the compound of the present disclosure showed improved results in terms of operation voltage, efficiency and lifetime by adjusting hole mobility to suit the device. In particular, the operation voltage was improved by about 9%, the luminous efficiency by about 8%, and the lifetime by about 30%. The comparative compound A can be used for the CPL, but it is not suitable for the HTL because of low hole transport efficiency.

The comparative compound D of Comparative Example 5, which has a structure in which a t-butylnaphthalene core has two substituents, is different from the compound of the present disclosure wherein the core has one substituent. This structure unnecessarily increases hole transport capacity, and fails to satisfy the hole mobility requirement for the device presented in the present disclosure. Consequently, it exhibited inferior results in terms of operation voltage, luminous efficiency and lifetime as compared to the device presented in the present disclosure. In particular, the lifetime was about 30% inferior.

The comparative compound B of Comparative Example 3 and the comparative compound C of Comparative Example 4 are different from the compound of the present disclosure because an arylamine group is directly bonded to the t-butylnaphthalene core. In the present disclosure, by introducing a linker with an appropriate length and strength between the core and the arylamine, the hole transport rate is adjusted to meet the device's requirement and a more stable compound is formed. The stabilized compound does not decompose or break down and, when applied to a device, it provides suitable properties as a hole transport layer and can effectively transport holes. This results in improved operation voltage, luminous efficiency and lifetime. In particular, the lifetime is improved by about 30% as compared to Comparative Examples 3 and 4.

The comparative compound E of Comparative Example 6 and the comparative compound F of Comparative Example 7 are different from the compound of the present disclosure in that a dimethylfluorene-based compound and an amine group are directly bonded.

The compounds containing methylfluorene substituents containing heteroaryl, aryl and aryl linkers proposed in the present disclosure have structures that can maintain appropriate charge transfer and have stable molecular arrangements can optimize these properties.

Unlike the compounds E and F, which contain dimethylfluorene-based substituents in which an amine group is directly bonded to the fluorene core, the methylphenylfluorene-based substituent bonded to the amine group via a phenyl group mediated by a methyl group effectively suppresses direct conjugation between the x-conjugated system of fluorene and the unshared electron pair of amine, thereby preventing excessive increase of HOMO and alleviating electron-donating. This partially non-conjugated structure is believed to provide hole transport characteristics suitable for the charge balance in the device presented in the present disclosure by improving oxidation stability and not excessively increasing hole injection and transport characteristics. In other words, the compound of the present disclosure exhibits hole transport characteristics more suitable for the device presented in the present disclosure than those of Comparative Examples 2 to 7 and provides superior effects in terms of operation, luminous efficiency and lifetime. In particular, the lifetime was improved by 30% as compared to Comparative Example 6, and by about 30% as compared to Comparative Example 7.

The comparative compound G of Comparative Example 8, which contains a phenyl group as R1 of Chemical Formula 1, is different from the compound of the present disclosure that contains a t-butyl group. Although the phenyl group possesses a large x-conjugated system, the charge transfer pathway can be dispersed because the electronic interaction is complex. This is believed to have resulted in a reduced hole transport capability as compared to the t-butyl group of the present disclosure, which has optimized electronic properties. In other words, the device presented in the present disclosure exhibited improved results in operation voltage, luminous efficiency and lifetime. The operation voltage was improved by about 10% and the lifetime was improved by about 30% as compared to Comparative Example 8.

### Test Example 2

### 1) Manufacturing of Organic Light-Emitting Device

A transparent electrode ITO thin film obtained from an OLED glass (Samsung Corning) was ultrasonically cleansed sequentially using trichloroethylene, acetone, ethanol and distilled water for 5 minutes respectively, and then stored in isopropanol until use. Then, the ITO substrate was mounted on the substrate folder of a vacuum deposition system, and 4,4′,4″-tris(N,N-(2-naphthyl)-phenylamino)triphenylamine (2-TNATA) was added to a cell of the vacuum deposition system.

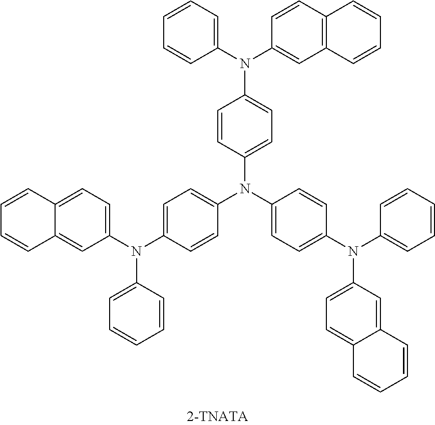 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Then, after evacuating the chamber until the vacuum level reached 10⁻⁶ torr, 2-TNATA was evaporated by applying a current to the cell, and a hole injection layer with a thickness of 600 Å was deposited on the ITO substrate. After adding N,N′-bis(α-naphthyl)-N,N′-diphenyl-4,4′-diamine (NPB) to another cell of the vacuum deposition system, NPB was evaporated by applying a current to the cell, and a hole transport layer with a thickness of 300 Å was deposited on the hole injection layer.

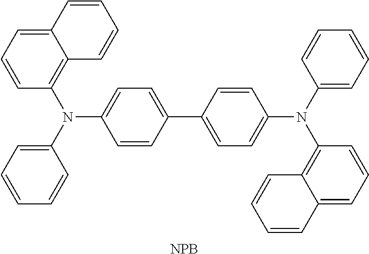 [SMILES: c1ccc(N(c2ccc(-c3ccc(N(c4ccccc4)c4cccc5ccccc45)cc3)cc2)c2cccc3ccccc23)cc1 ; "NPB"]

After the hole injection layer and the hole transport layer were formed, blue light-emitting materials with the following structures were deposited thereon as a light-emitting layer. Specifically, in one cell of the vacuum deposition system, a blue light-emitting host material H1 was vacuum-deposited to a thickness of 200 Å, and 5% of a blue light-emitting dopant material D1 was vacuum-deposited thereon based on the host material.

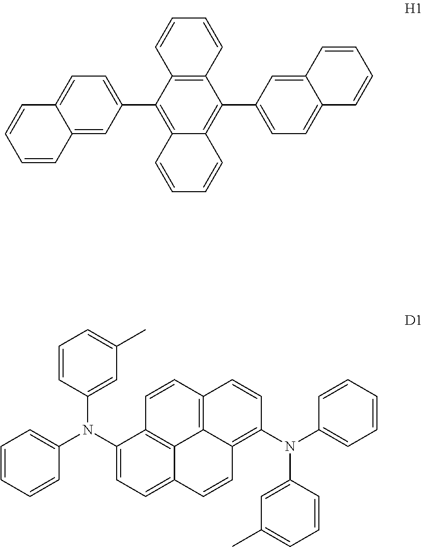

Subsequently, a compound of Structural Formula E1 was deposited to a thickness of 300 Å as an electron transport layer.

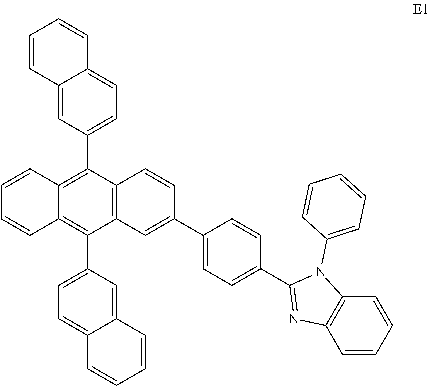 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

After depositing lithium fluoride (LiF) to a thickness of 10 Å as an electron injection layer, an OLED device was manufactured by depositing an Al negative electrode to a thickness of 1,000 Å. All the organic compounds required for the manufacturing of the OLED device were purified by vacuum sublimation at 10⁻⁸ to 10⁻⁶ torr. Organic light-emitting devices were manufactured using in the same manner as in Test Example 2, except that the electron blocking layer was formed to a thickness of 50 Å using the compounds described in Table 6 after the hole transport layer was formed to a thickness of 250 Å using NPB. The result of measuring the operation voltage, luminous efficiency, color coordinates (CIE) and lifetime of the blue organic light-emitting devices manufactured according to the present disclosure is shown in Table 6.

TABLE 6

| | | Operation | Luminous | | |
|---|---|---|---|---|---|
| | | voltage | efficiency | CIE | Lifetime |
| No. | Compound | (V) | (cd/A) | (x, y) | (T₉₅) |
| Example 54 | 001 | 7.25 | 69.63 | (0.215, 0.419) | 89 |
| Example 55 | 036 | 7.19 | 69.78 | (0.216, 0.424) | 91 |
| Example 56 | 054 | 7.21 | 68.84 | (0.214, 0.425) | 89 |
| Example 57 | 062 | 7.18 | 69.31 | (0.214, 0.418) | 86 |
| Example 58 | 096 | 7.20 | 69.67 | (0.214, 0.418) | 88 |
| Example 59 | 108 | 7.24 | 67.50 | (0.215, 0.417) | 85 |
| Example 60 | 120 | 7.16 | 69.08 | (0.209, 0.422) | 86 |
| Example 61 | 130 | 7.23 | 68.98 | (0.210, 0.428) | 88 |
| Example 62 | 142 | 7.24 | 68.16 | (0.209, 0.428) | 87 |
| Example 63 | 171 | 7.20 | 68.83 | (0.210, 0.430) | 90 |
| Example 64 | 178 | 7.16 | 70.21 | (0.212, 0.428) | 87 |
| Example 65 | 223 | 7.18 | 69.02 | (0.215, 0.424) | 91 |
| Example 66 | 234 | 7.08 | 68.19 | (0.214, 0.422) | 88 |
| Example 67 | 249 | 7.14 | 70.15 | (0.215, 0.417) | 86 |
| Example 68 | 261 | 7.23 | 68.69 | (0.214, 0.420) | 88 |
| Example 69 | 288 | 7.16 | 69.22 | (0.214, 0.418) | 89 |
| Example 70 | 293 | 7.25 | 69.12 | (0.214, 0.418) | 90 |
| Example 71 | 303 | 7.24 | 68.85 | (0.215, 0.419) | 88 |
| Example 72 | 305 | 7.26 | 67.98 | (0.214, 0.426) | 90 |
| Example 73 | 325 | 7.22 | 70.22 | (0.215, 0.424) | 86 |
| Example 74 | 331 | 7.21 | 69.51 | (0.219, 0.422) | 89 |
| Example 75 | 358 | 7.17 | 70.56 | (0.214, 0.418) | 88 |
| Example 76 | 380 | 7.23 | 69.87 | (0.213, 0.417) | 89 |
| Example 77 | 383 | 7.21 | 68.65 | (0.213, 0.420) | 85 |
| Example 78 | 396 | 7.25 | 69.11 | (0.215, 0.420) | 87 |
| Example 79 | 403 | 7.26 | 69.14 | (0.215, 0.420) | 89 |
| Example 80 | 426 | 7.23 | 68.87 | (0.214, 0.420) | 85 |
| Example 81 | 455 | 7.24 | 68.92 | (0.215, 0.418) | 87 |
| Example 82 | 480 | 7.15 | 68.79 | (0.214, 0.420) | 87 |
| Example 83 | 489 | 7.18 | 69.77 | (0.215, 0.421) | 91 |
| Example 84 | 508 | 7.22 | 70.13 | (0.217, 0.422) | 90 |
| Example 85 | 518 | 7.15 | 68.58 | (0.214, 0.418) | 86 |
| Example 86 | 559 | 7.26 | 69.74 | (0.213, 0.417) | 89 |
| Example 87 | 573 | 7.14 | 68.81 | (0.214, 0.420) | 90 |
| Example 88 | 590 | 7.17 | 69.22 | (0.211, 0.420) | 86 |
| Example 89 | 610 | 7.09 | 68.67 | (0.213, 0.430) | 85 |
| Example 90 | 617 | 7.29 | 66.43 | (0.212, 0.426) | 90 |
| Example 91 | 630 | 7.24 | 69.31 | (0.215, 0.420) | 87 |
| Example 92 | 659 | 7.23 | 68.61 | (0.213, 0.420) | 91 |
| Example 93 | 675 | 7.15 | 69.10 | (0.214, 0.418) | 86 |
| Example 94 | 717 | 7.21 | 70.04 | (0.213, 0.420) | 93 |
| Example 95 | 719 | 7.16 | 68.98 | (0.215, 0.420) | 95 |
| Example 96 | 720 | 7.19 | 69.84 | (0.214, 0.420) | 95 |
| Example 97 | 737 | 7.20 | 69.90 | (0.213, 0.420) | 87 |
| Example 98 | 740 | 7.22 | 69.82 | (0.214, 0.420) | 88 |
| Example 99 | 752 | 7.24 | 68.75 | (0.214, 0.420) | 90 |
| Example 100 | 772 | 7.22 | 68.84 | (0.214, 0.422) | 91 |
| Example 101 | 776 | 7.18 | 68.79 | (0.214, 0.420) | 89 |
| Example 102 | 791 | 7.21 | 69.05 | (0.213, 0.420) | 90 |
| Example 103 | 807 | 7.14 | 69.68 | (0.214, 0.420) | 92 |
| Example 104 | 812 | 7.08 | 70.11 | (0.214, 0.420) | 95 |
| Example 105 | 815 | 7.15 | 70.08 | (0.215, 0.421) | 97 |
| Example 106 | 835 | 7.13 | 69.97 | (0.214, 0.420) | 96 |
| Comparative | TmPyPB | 8.20 | 57.71 | (0.211, 0.430) | 65 |
| Example 9 | | | | | |
| Comparative | Comparative | 7.72 | 57.32 | (0.211, 0.427) | 63 |
| Example 10 | compound A | | | | |
| Comparative | Comparative | 7.67 | 56.96 | (0.210, 0.429) | 59 |
| Example 11 | compound B | | | | |
| Comparative | Comparative | 7.69 | 57.84 | (0.210, 0.427) | 61 |
| Example 12 | compound C | | | | |
| Comparative | Comparative | 7.70 | 58.09 | (0.213, 0.424) | 57 |
| Example 13 | compound D | | | | |
| Comparative | Comparative | 7.64 | 58.75 | (0.212, 0.429) | 69 |
| Example 14 | compound E | | | | |
| Comparative | Comparative | 7.61 | 58.47 | (0.212, 0.428) | 68 |
| Example 15 | compound F | | | | |
| Comparative | Comparative | 7.65 | 57.69 | (0.211, 0.427) | 60 |
| Example 16 | compound G | | | | |

As can be seen from Table 6, the blue organic light-emitting device using the electron blocking layer material of the present disclosure showed a lower operation voltage and significantly improved luminous efficiency and lifetime as compared to Comparative Examples 9 to 16. Since the comparative compound A of Comparative Example 10 has a symmetric molecular structure, uniform thin film formation is difficult due to poor film-forming property. Therefore, it is not suitable for the characteristics of the device presented in the present disclosure. In particular, the lifetime was inferior as about 25%. In addition, the present disclosure effectively blocks electrons due to the large difference in HOMO and LUMO energy levels, thereby maximizing exciton formation in the light-emitting layer. In contrast, the comparative compound A of Comparative Example 10 lacks this electron-blocking ability. Thus, although it can be used for a capping layer, it is not suitable for use in the electron blocking layer.

If unbound electrons in the light-emitting layer move to the positive electrode through the hole transport layer, the efficiency and lifetime of the OLED device decrease problematically. If a compound having high LUMO and T1 levels is used for the electron blocking layer to prevent this phenomenon, the probability that electrons moving toward the positive electrode through the light-emitting layer form excitons in the light-emitting layer increases. As compared to the comparative compound B of Comparative Example 11 and the comparative compound C of Comparative Example 12, wherein arylamine is directly bonded to the t-butylnaphthalene core, the compound of the present disclosure having a linker has a relatively shallow LUMO level, and the probability of exciton formation in the light-emitting layer is increased. As a result, the device presented in the present disclosure showed improved operation voltage, luminous efficiency and lifetime as compared to Comparative Example 11 and Comparative Example 12. In particular, the lifetime was improved by about 30%.

In addition, unlike the comparative compounds D, E and F of Comparative Examples 13, 14 and 15, wherein dimethylfluorene-based substituents are bonded directly, the compound of the present disclosure, which contains a methylphenyl fluorene-based substituent and a heteroaryl group, is thought to increase the stability and efficiency of the device by stabilizing the HOMO level and optimizing electron distribution of the molecule, thereby reducing charge leakage and non-radiative recombination at the interface.

As a result, the device presented in the present disclosure showed improved operation voltage, luminous efficiency and lifetime as compared to Comparative Examples 11 and 12. In particular, the lifetime was improved by 30% as compared to Comparative Example 13, and about 20% as compared to Comparative Examples 14 and 15.

The comparative compound G of Comparative Example 16, which has a phenyl group as R1 of Chemical Formula 1, is different form the compound of the present disclosure containing a t-butyl group. Although the phenyl group of the comparative compound G is sensitive to oxidation, the t-butyl group of the present disclosure is advantageous in that it is resistant to oxidation and thermal decomposition because it is chemically stable. This is believed to have resulted in the long-term stability of the compound of the present disclosure. That is to say, it improved the operation voltage, luminous efficiency and lifetime of the device presented in the present disclosure. In particular, the lifetime was improved by about 30% as compared to Comparative Example 16.

In conclusion, the compound of the present disclosure was confirmed to provide superiority in all aspects of operation, efficiency and lifetime as compared to Comparative Examples 1 to 16.

### DETAILED DESCRIPTION OF MAIN ELEMENTS

100: substrate
200: positive electrode
300: organic layer
301: hole injection layer
302: hole transport layer
303: light-emitting layer
304: electron transport layer
305: electron injection layer
306: electron blocking layer
400: negative electrode

What is claimed is:

1. A compound represented by Chemical Formula 1: 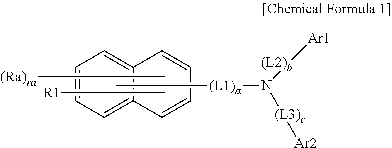
in Chemical Formula 1,
R1 is a substituted or unsubstituted C₁ to C₆₀ branched alkyl group,
L1 is a substituted or unsubstituted C₆ to C₆₀ arylene group,
a is an integer from 1 to 3, wherein if a is 2 or greater, L1's are identical or different,
L2 and L3, which are identical or different, are independently a direct bond; or a substituted or unsubstituted C₆ to C₆₀ arylene group,
b and c are respectively an integer from 0 to 3, wherein if b is 2 or greater, L2's are identical or different, and if c is 2 or greater, L3's are identical or different,
Ra is hydrogen; deuterium; a halogen group; a cyano group; a substituted or unsubstituted C₁ to C₆₀ alkyl group; a substituted or unsubstituted C₆ to C₆₀ aryl group; or a substituted or unsubstituted C₂ to C₆₀ heteroaryl group,
ra is an integer from 0 to 6, wherein if ra is 2 or greater, Ra's are identical or different,
with the proviso that L1 and R1 are not bonded at para positions,
Ar1 and Ar2, which are identical or different, are independently a substituted or unsubstituted C₄ to C₆₀ monocyclic or polycyclic aliphatic ring group; a substituted or unsubstituted C₆ to C₆₀ aryl group consisting of one or more benzene ring; a substituted or unsubstituted C₂ to C₆₀ tricyclic heteroaryl group; or Structural Formula A, 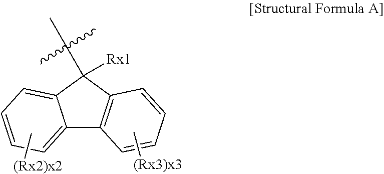
in Structural Formula A, 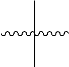
is a moiety connected to Chemical Formula 1,
Rx1 is a substituted or unsubstituted C₁ to C₆₀ alkyl group; a substituted or unsubstituted C₄ to C₆₀ monocyclic or polycyclic aliphatic ring group; or a substituted or unsubstituted C₆ to C₆₀ aryl group,
Rx2 and Rx3 are hydrogen; deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group, or two or more adjacent substituents are bonded to each other to form a substituted or unsubstituted ring, and
x2 and x3 are integers from 0 to 4, wherein if x2 and x3 are respectively 2 or greater, the substituents in the parentheses are identical or different.

2. The compound according to claim 1, wherein Chemical Formula 1 is represented by any one of Chemical Formulas 1-1 to 1-13: 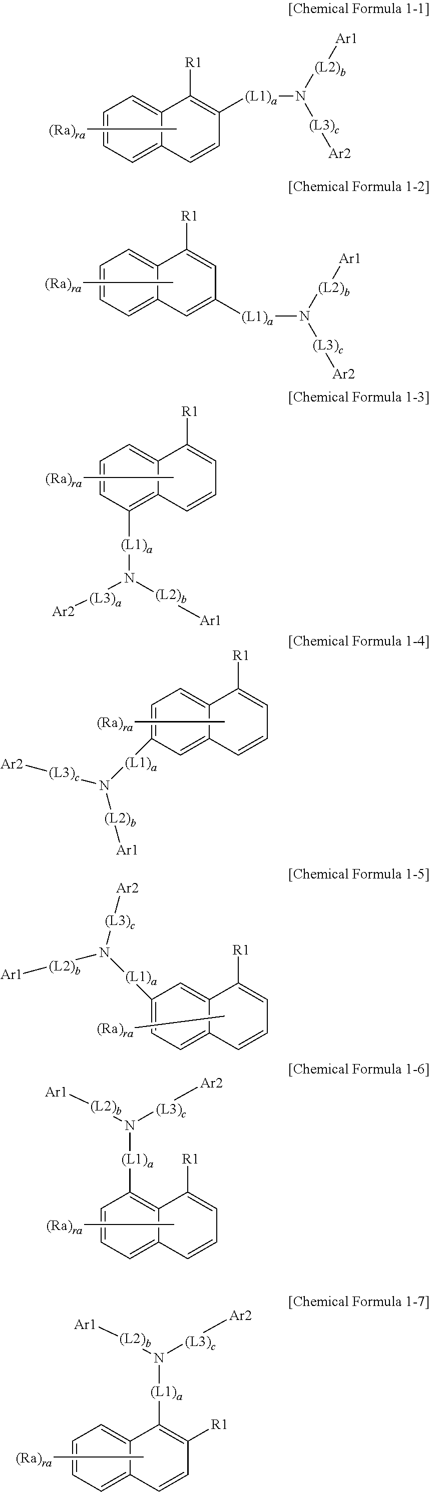 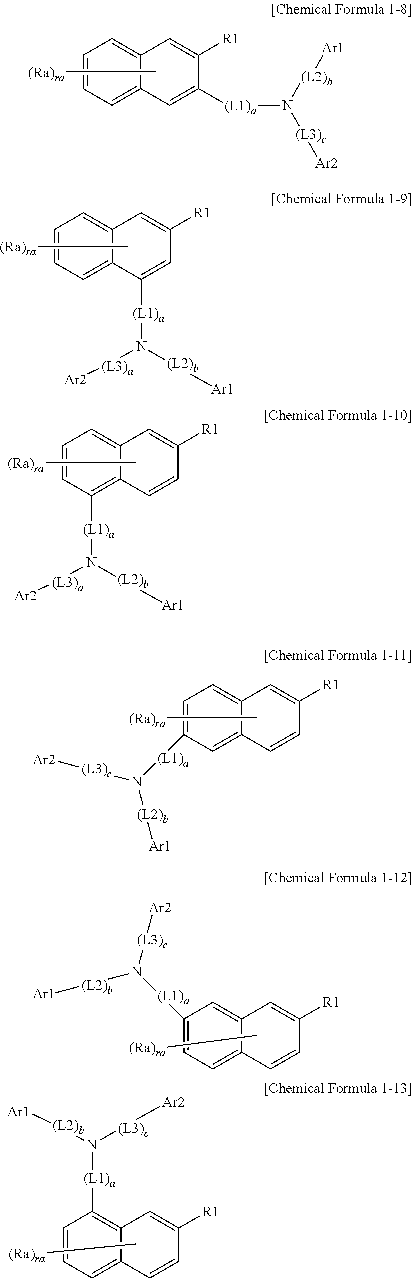
in Chemical Formulas 1-1 to 1-13,
L1, L2, L3, Ar1, Ar2, R1, Ra, a, b, c and ra are the same as defined above in claim 1.

3. The compound according to claim 1, wherein 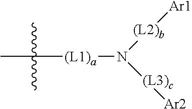
of Chemical Formula 1 is represented by any one of Chemical Formulas A to E: 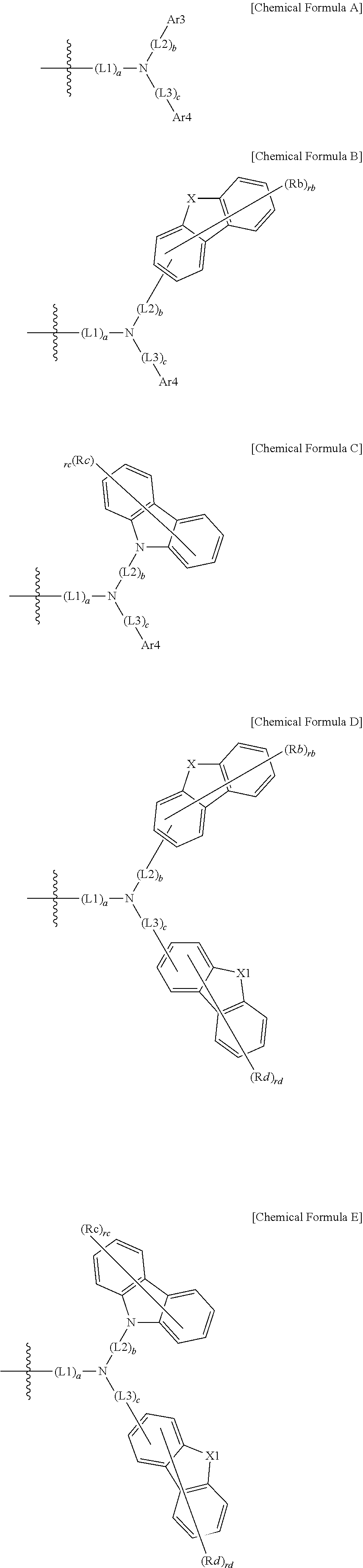
in Chemical Formulas A to E,
L1, L2, L3, a, b and c are the same as defined above in claim 1,
X is O; S; or NR,
R is hydrogen; deuterium; a substituted or unsubstituted C₁ to C₆₀ alkyl group; or a substituted or unsubstituted C₆ to C₆₀ aryl group,
X1 is O; or S,
Rb, Rc and Rd, which are identical or different, are independently hydrogen; deuterium; a halogen group; a cyano group; a substituted or unsubstituted C₁ to C₆₀ alkyl group; a substituted or unsubstituted C₆ to C₆₀ aryl group; or a substituted or unsubstituted C₂ to C₆₀ heteroaryl group,
rb and rd are respectively an integer from 0 to 7, wherein if rb is 2 or greater, Rb's are identical or different, and if rd is 2 or greater, Rd's are identical or different,
rc is an integer from 0 to 8, wherein if rc is 2 or greater, Rc's are identical or different, and
Ar3 and Ar4, which are identical or different, are independently a substituted or unsubstituted cyclopentyl group; a substituted or unsubstituted cyclohexyl group; a substituted or unsubstituted adamantyl group; a substituted or unsubstituted norbornyl group; a substituted or unsubstituted benzonorbornyl group; a substituted or unsubstituted indanyl group; a substituted or unsubstituted tetrahydronaphthyl group; a substituted or unsubstituted spiro[4.5]decanyl group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; or Structural Formula A.

4. The compound according to claim 1, wherein Ar1 and Ar2, which are identical or different, are independently a substituted or unsubstituted cyclopentyl group; a substituted or unsubstituted cyclohexyl group; a substituted or unsubstituted adamantyl group; a substituted or unsubstituted norbornyl group; a substituted or unsubstituted benzonorbornyl group; a substituted or unsubstituted indanyl group; a substituted or unsubstituted tetrahydronaphthyl group; a substituted or unsubstituted spiro[4.5]decanyl group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted phenanthrene group; a substituted or unsubstituted dibenzofuran group; a substituted or unsubstituted dibenzothiophene group; a substituted or unsubstituted carbazole group; or Structural Formula A, and
Structural Formula A is any one of Structural Formulas A-1 to A-4: 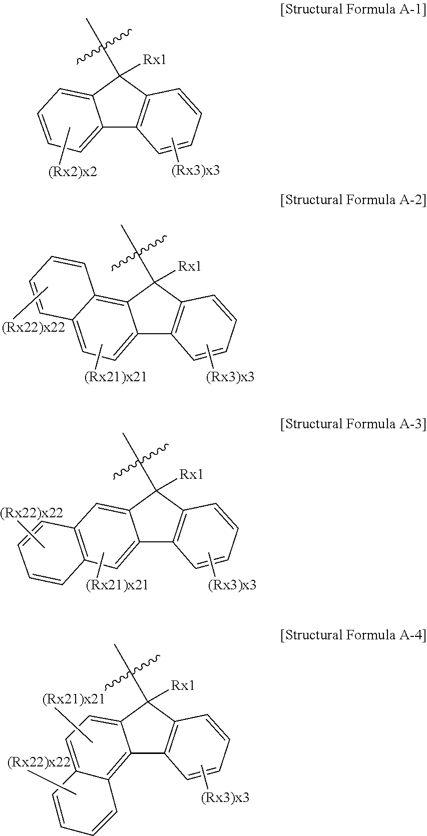
in Structural Formulas A-1 to A-4,
Rx21 and Rx22, which are identical or different, are independently, hydrogen;
deuterium; a cyano group; a substituted or unsubstituted C₁ to C₆₀ alkyl group; a substituted or unsubstituted C₄ to C₆₀ monocyclic or polycyclic aliphatic ring group; a substituted or unsubstituted C₆ to C₆₀ aryl group; or a substituted or unsubstituted C₂ to C₆₀ heteroaryl group,
x21 is an integer from 0 to 2, wherein if x21 is 2 or greater, the substituents in the parentheses are identical or different,
x22 is an integer from 0 to 4, wherein if x22 is 2 or greater, the substituents in the parentheses are identical or different, and
Rx1 to Rx3, x2 and x3 are the same as defined in Structural Formula A.

5. The compound according to claim 1, wherein R1 is represented by Chemical Formula H-1: 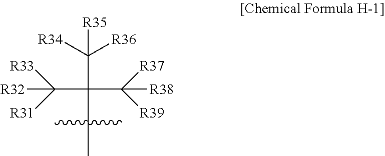
in Chemical Formula H-1,
R31 to R39, which are identical or different, are independently hydrogen; or deuterium.

6. The compound according to claim 1, wherein the compound of Chemical Formula 1 has a deuterium substitution rate of 0% or 1% to 100%.

7. The compound according to claim 1, wherein Chemical Formula 1 is represented by any one of the following compounds:

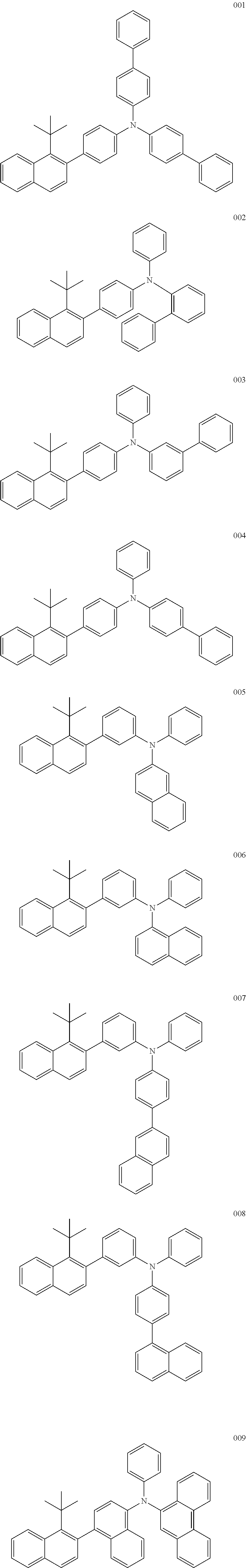

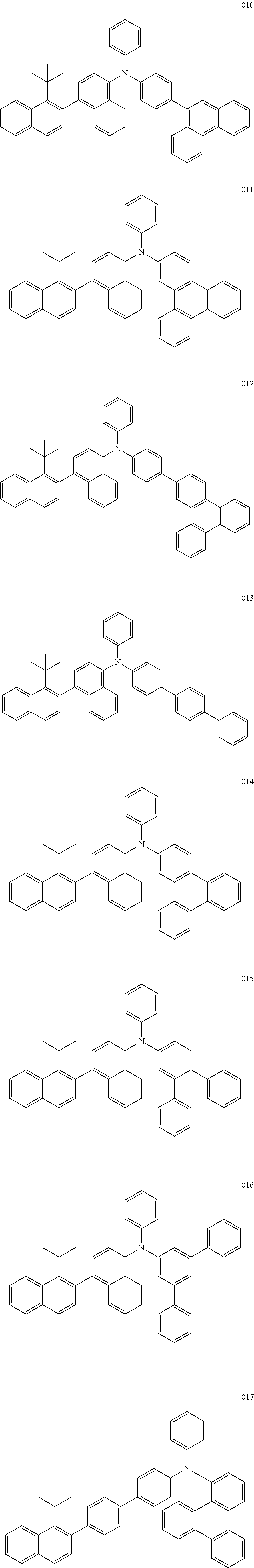

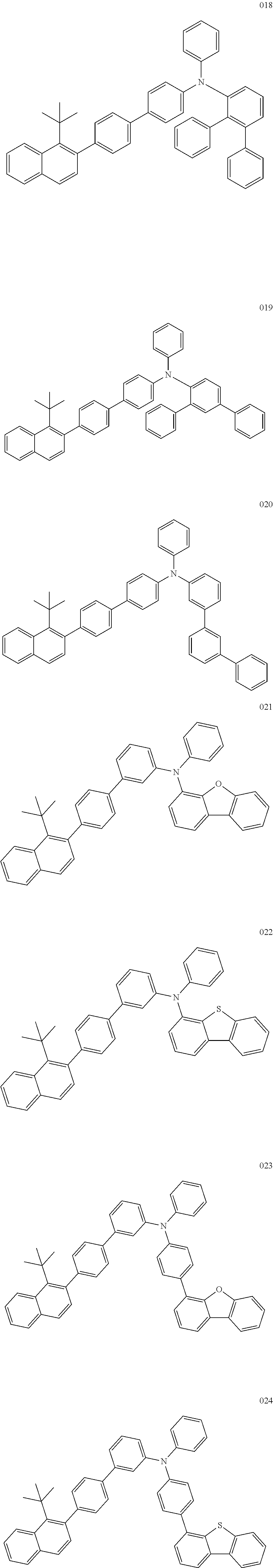

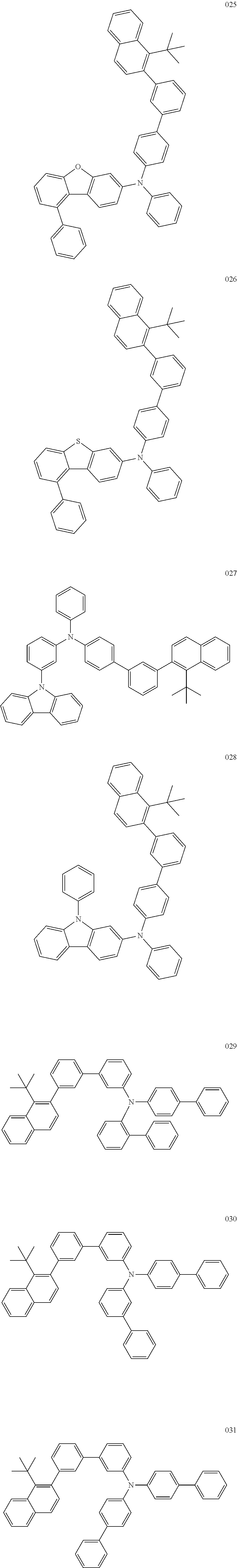

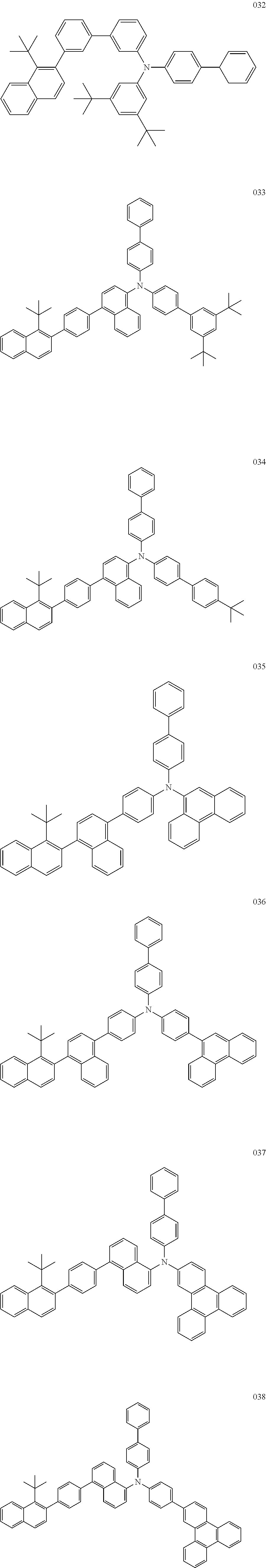

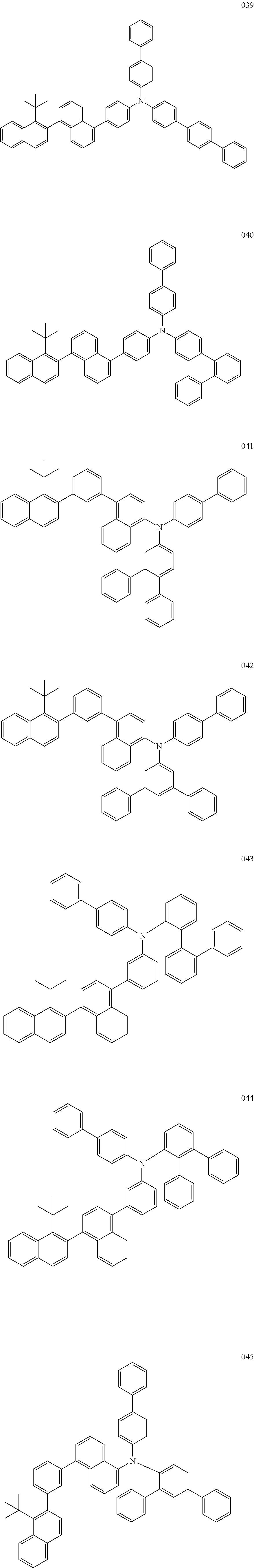

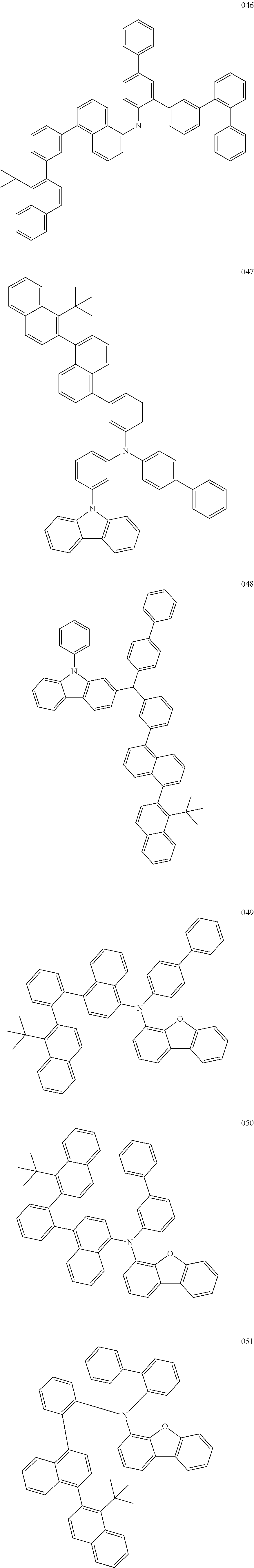

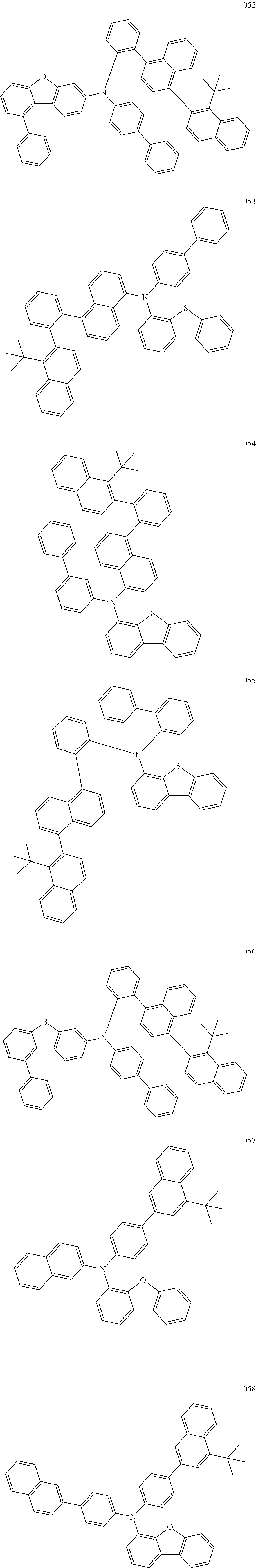

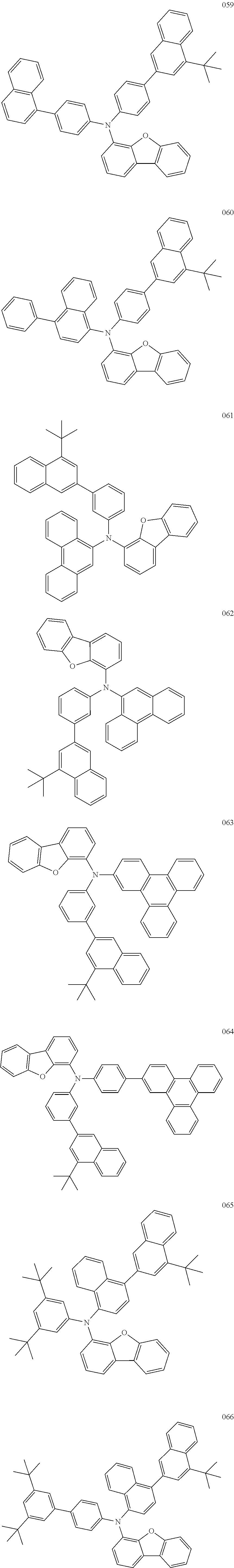

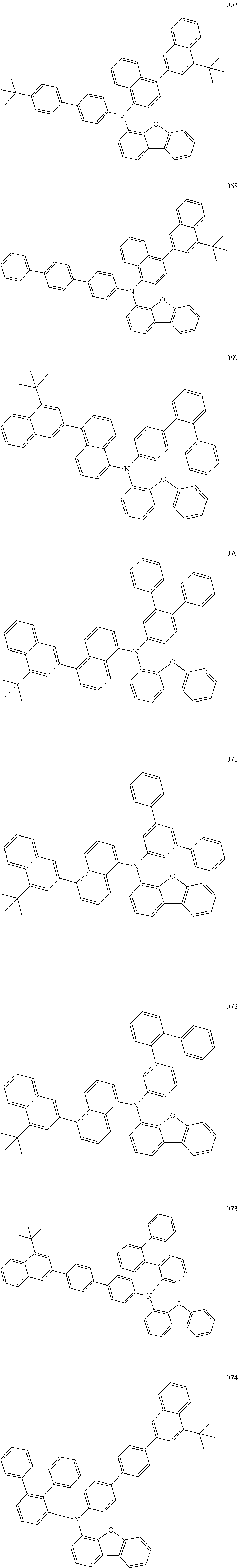

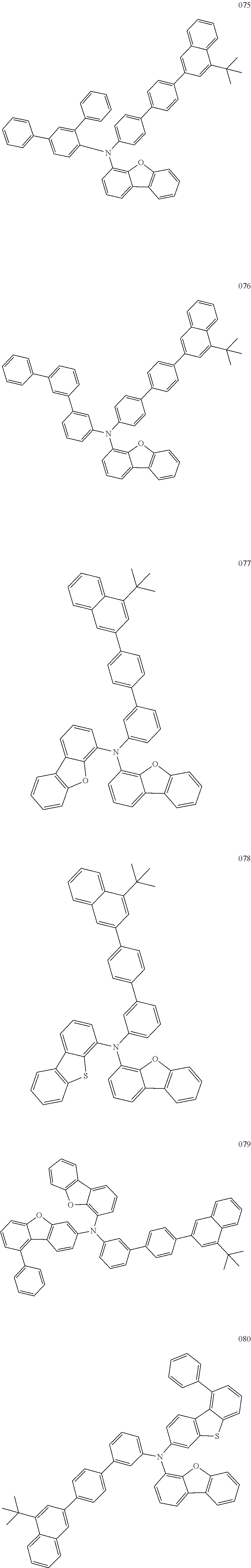

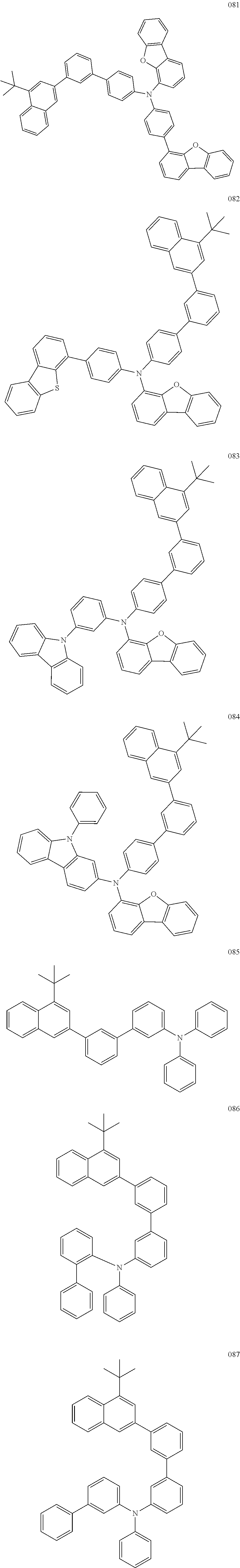

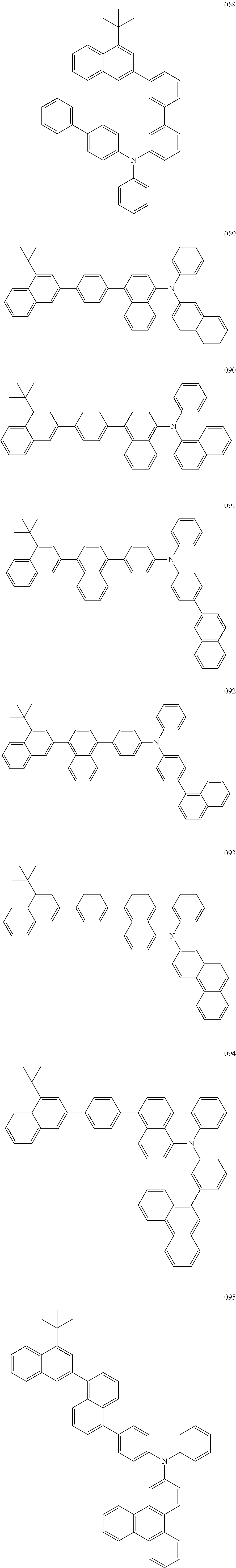

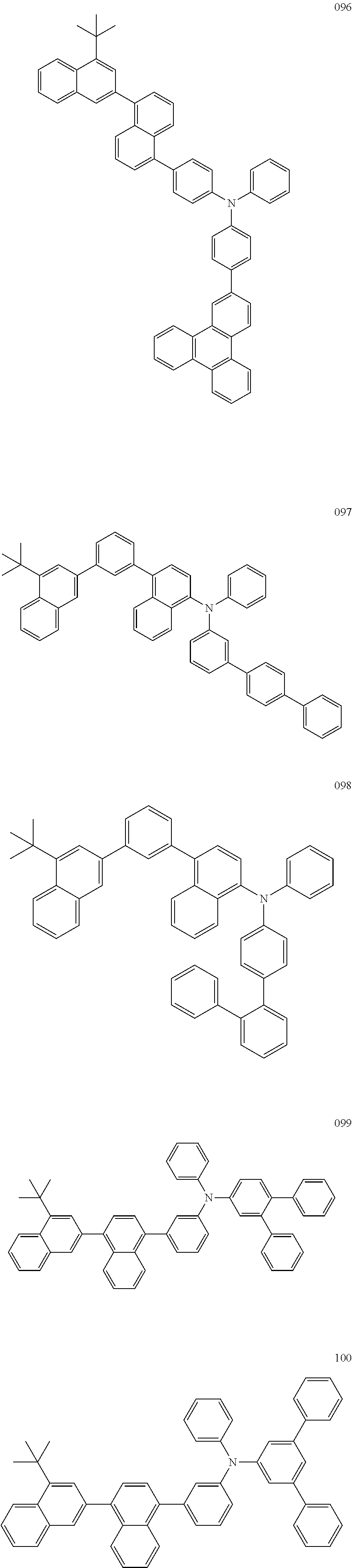 [SMILES: CC(C)(C)c1cc(-c2cccc3c(-c4ccc(N(c5ccccc5)c5ccc(-c6ccc7c8ccccc8c8ccccc8c7c6)cc5)cc4)cccc23)cc2ccccc12 ; CC(C)(C)c1cc(-c2cccc(-c3ccc(N(c4ccccc4)c4cccc(-c5ccc(-c6ccccc6)cc5)c4)c4ccccc34)c2)cc2ccccc12 ; CC(C)(C)c1cc(-c2cccc(-c3ccc(N(c4ccccc4)c4ccc(-c5ccccc5-c5ccccc5)cc4)c4ccccc34)c2)cc2ccccc12 ; CC(C)(C)c1cc(-c2ccc(-c3cccc(N(c4ccccc4)c4ccc(-c5ccccc5)c(-c5ccccc5)c4)c3)c3ccccc23)cc2ccccc12 ; CC(C)(C)c1cc(-c2ccc(-c3cccc(N(c4ccccc4)c4cc(-c5ccccc5)cc(-c5ccccc5)c4)c3)c3ccccc23)cc2ccccc12]

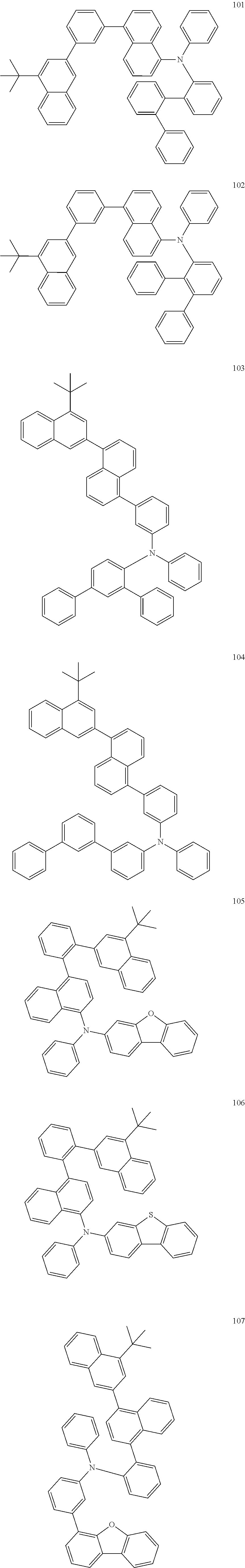

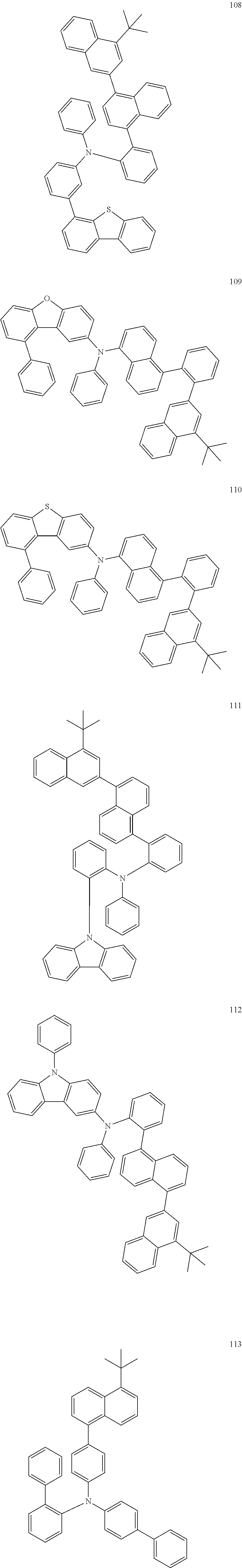

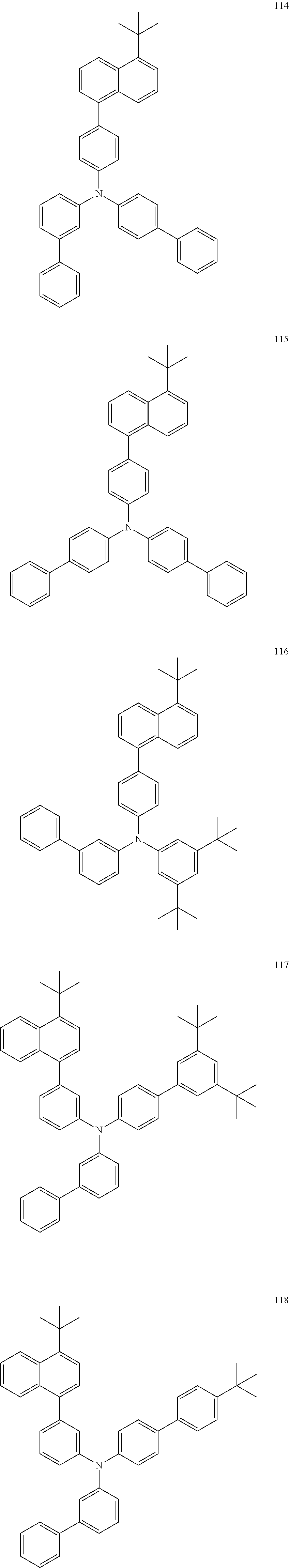 [SMILES: CC(C)(C)c1cccc2c(-c3ccc(N(c4ccc(-c5ccccc5)cc4)c4cccc(-c5ccccc5)c4)cc3)cccc12 ; CC(C)(C)c1cccc2c(-c3ccc(N(c4ccc(-c5ccccc5)cc4)c4ccc(-c5ccccc5)cc4)cc3)cccc12 ; CC(C)(C)c1cc(N(c2ccc(-c3cccc4c(C(C)(C)C)cccc34)cc2)c2cccc(-c3ccccc3)c2)cc(C(C)(C)C)c1 ; CC(C)(C)c1cc(-c2ccc(N(c3cccc(-c4ccccc4)c3)c3cccc(-c4ccc(C(C)(C)C)c5ccccc45)c3)cc2)cc(C(C)(C)C)c1 ; CC(C)(C)c1ccc(-c2ccc(N(c3cccc(-c4ccccc4)c3)c3cccc(-c4ccc(C(C)(C)C)c5ccccc45)c3)cc2)cc1]

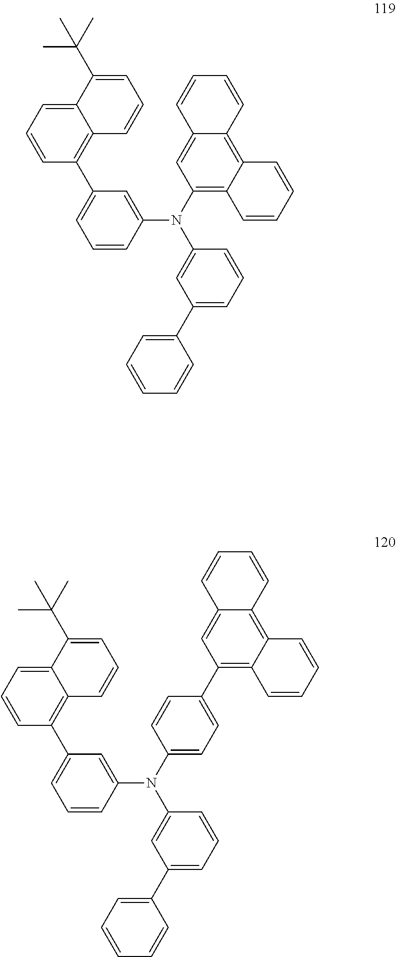 [SMILES: CC(C)(C)c1cccc2c(-c3cccc(N(c4cccc(-c5ccccc5)c4)c4cc5ccccc5c5ccccc45)c3)cccc12 ; CC(C)(C)c1cccc2c(-c3cccc(N(c4ccc(-c5cc6ccccc6c6ccccc56)cc4)c4cccc(-c5ccccc5)c4)c3)cccc12]

[chemical structure]

[chemical structure]

[chemical structure]

[chemical structure]

[chemical structure]

[chemical structure]

[chemical structure]

[chemical structure]

[chemical structure]

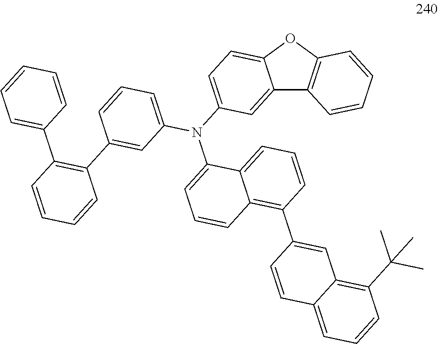 [SMILES: CC(C)(C)c1cccc2ccc(-c3cccc4c(N(c5cccc(-c6ccccc6-c6ccccc6)c5)c5ccc6oc7ccccc7c6c5)cccc34)cc12]

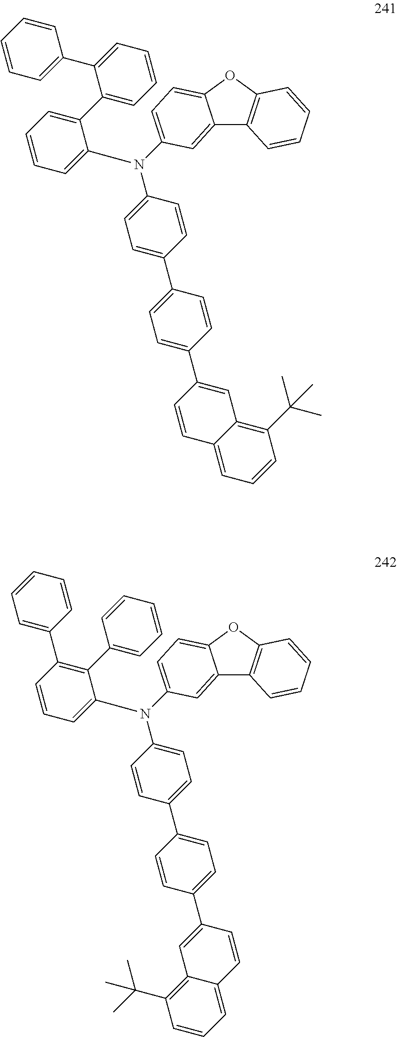 [SMILES: CC(C)(C)c1cccc2ccc(-c3ccc(-c4ccc(N(c5ccc6oc7ccccc7c6c5)c5ccccc5-c5ccccc5-c5ccccc5)cc4)cc3)cc12 ; CC(C)(C)c1cccc2ccc(-c3ccc(-c4ccc(N(c5ccc6oc7ccccc7c6c5)c5cccc(-c6ccccc6)c5-c5ccccc5)cc4)cc3)cc12]

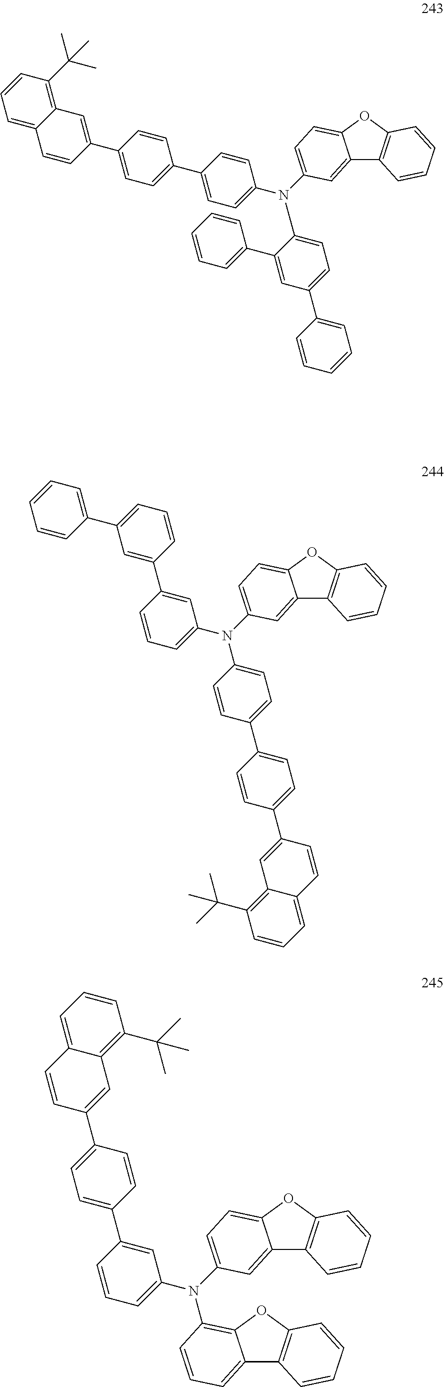 [SMILES: CC(C)(C)c1cccc2ccc(-c3ccc(-c4ccc(N(c5ccc6oc7ccccc7c6c5)c5ccc(-c6ccccc6)cc5-c5ccccc5)cc4)cc3)cc12 ; CC(C)(C)c1cccc2ccc(-c3ccc(-c4ccc(N(c5cccc(-c6cccc(-c7ccccc7)c6)c5)c5ccc6oc7ccccc7c6c5)cc4)cc3)cc12 ; CC(C)(C)c1cccc2ccc(-c3ccc(-c4cccc(N(c5ccc6oc7ccccc7c6c5)c5cccc6c5oc5ccccc56)c4)cc3)cc12]

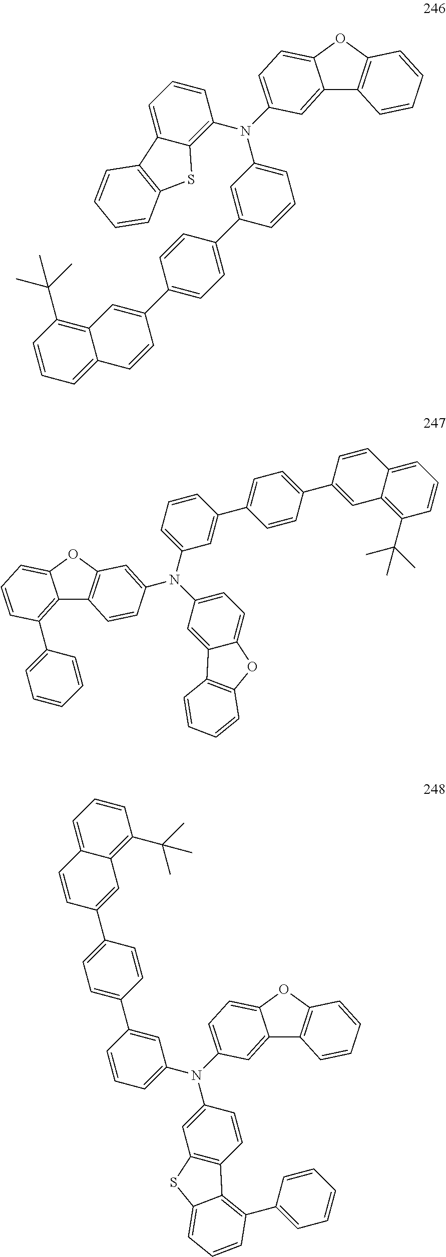 [SMILES: CC(C)(C)c1cccc2ccc(-c3ccc(-c4cccc(N(c5ccc6oc7ccccc7c6c5)c5cccc6c5sc5ccccc56)c4)cc3)cc12 ; CC(C)(C)c1cccc2ccc(-c3ccc(-c4cccc(N(c5ccc6c(c5)oc5cccc(-c7ccccc7)c56)c5ccc6oc7ccccc7c6c5)c4)cc3)cc12 ; CC(C)(C)c1cccc2ccc(-c3ccc(-c4cccc(N(c5ccc6c(c5)sc5cccc(-c7ccccc7)c56)c5ccc6oc7ccccc7c6c5)c4)cc3)cc12]

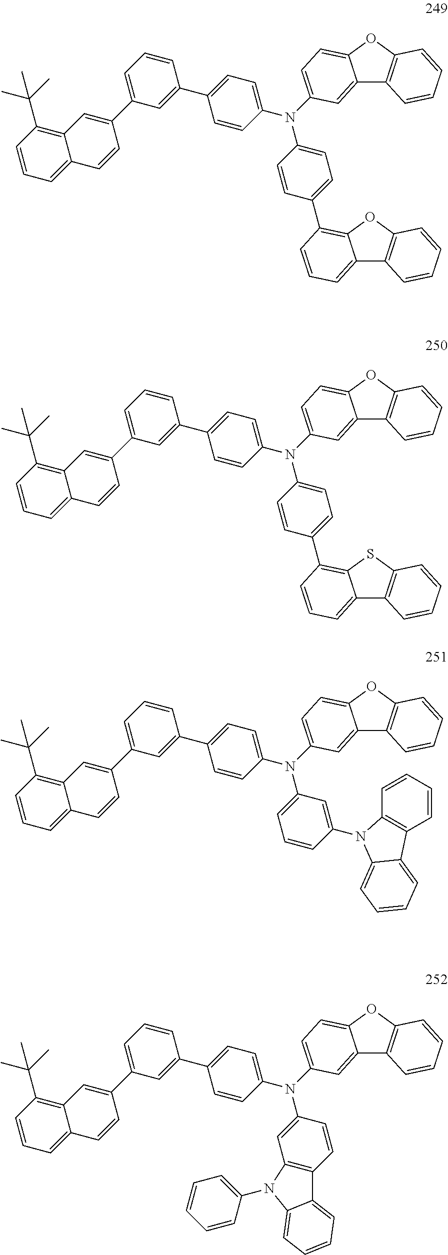 [SMILES: CC(C)(C)c1cccc2ccc(-c3cccc(-c4ccc(N(c5ccc(-c6cccc7c6oc6ccccc67)cc5)c5ccc6oc7ccccc7c6c5)cc4)c3)cc12 ; CC(C)(C)c1cccc2ccc(-c3cccc(-c4ccc(N(c5ccc(-c6cccc7c6sc6ccccc67)cc5)c5ccc6oc7ccccc7c6c5)cc4)c3)cc12 ; CC(C)(C)c1cccc2ccc(-c3cccc(-c4ccc(N(c5cccc(-n6c7ccccc7c7ccccc76)c5)c5ccc6oc7ccccc7c6c5)cc4)c3)cc12 ; CC(C)(C)c1cccc2ccc(-c3cccc(-c4ccc(N(c5ccc6oc7ccccc7c6c5)c5ccc6c7ccccc7n(-c7ccccc7)c6c5)cc4)c3)cc12]

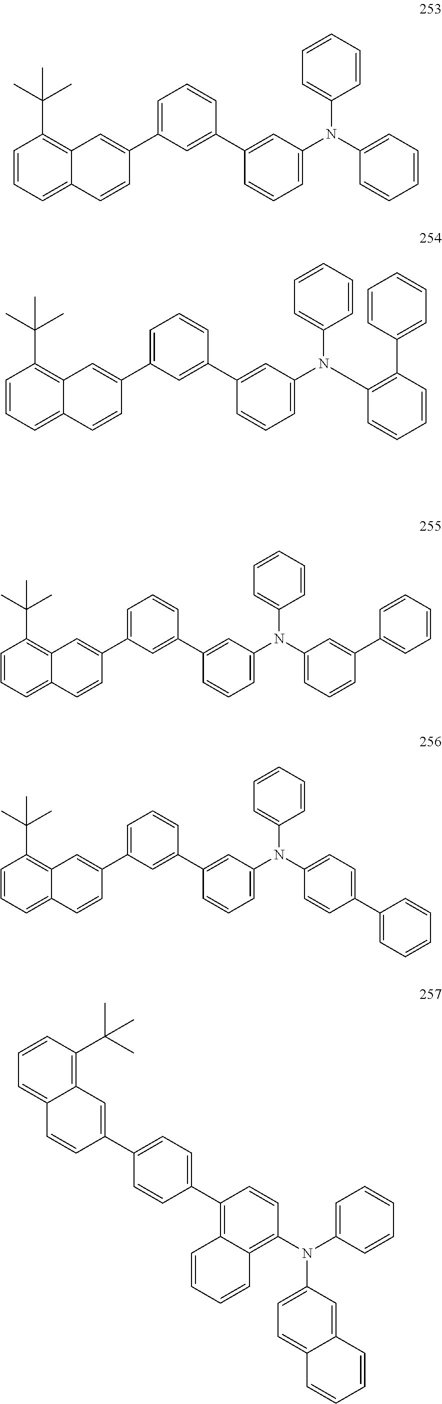 [SMILES: CC(C)(C)c1cccc2ccc(-c3cccc(-c4cccc(N(c5ccccc5)c5ccccc5)c4)c3)cc12 ; CC(C)(C)c1cccc2ccc(-c3cccc(-c4cccc(N(c5ccccc5)c5ccccc5-c5ccccc5)c4)c3)cc12 ; CC(C)(C)c1cccc2ccc(-c3cccc(-c4cccc(N(c5ccccc5)c5cccc(-c6ccccc6)c5)c4)c3)cc12 ; CC(C)(C)c1cccc2ccc(-c3cccc(-c4cccc(N(c5ccccc5)c5ccc(-c6ccccc6)cc5)c4)c3)cc12 ; CC(C)(C)c1cccc2ccc(-c3ccc(-c4ccc(N(c5ccccc5)c5ccc6ccccc6c5)c5ccccc45)cc3)cc12]

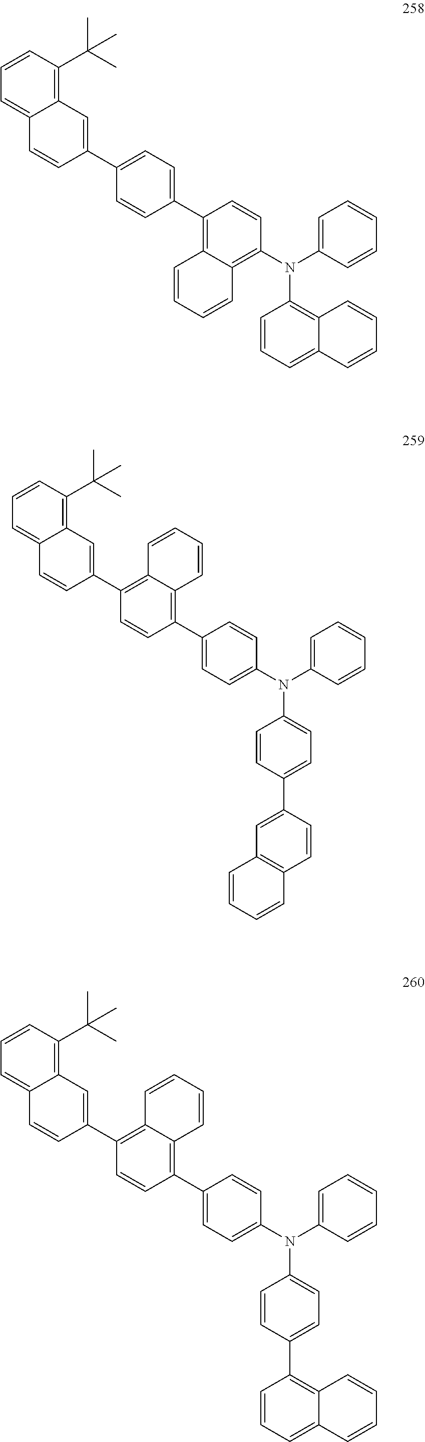 [SMILES: CC(C)(C)c1cccc2ccc(-c3ccc(-c4ccc(N(c5ccccc5)c5cccc6ccccc56)c5ccccc45)cc3)cc12 ; CC(C)(C)c1cccc2ccc(-c3ccc(-c4ccc(N(c5ccccc5)c5ccc(-c6ccc7ccccc7c6)cc5)cc4)c4ccccc34)cc12 ; CC(C)(C)c1cccc2ccc(-c3ccc(-c4ccc(N(c5ccccc5)c5ccc(-c6cccc7ccccc67)cc5)cc4)c4ccccc34)cc12]

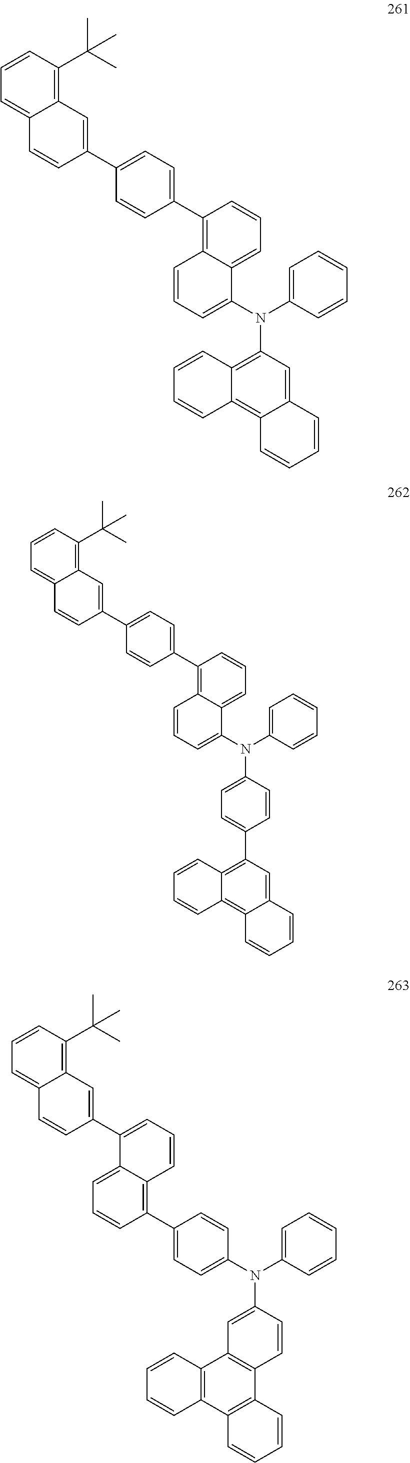 [SMILES: CC(C)(C)c1cccc2ccc(-c3ccc(-c4cccc5c(N(c6ccccc6)c6cc7ccccc7c7ccccc67)cccc45)cc3)cc12 ; CC(C)(C)c1cccc2ccc(-c3ccc(-c4cccc5c(N(c6ccccc6)c6ccc(-c7cc8ccccc8c8ccccc78)cc6)cccc45)cc3)cc12 ; CC(C)(C)c1cccc2ccc(-c3cccc4c(-c5ccc(N(c6ccccc6)c6ccc7c8ccccc8c8ccccc8c7c6)cc5)cccc34)cc12]

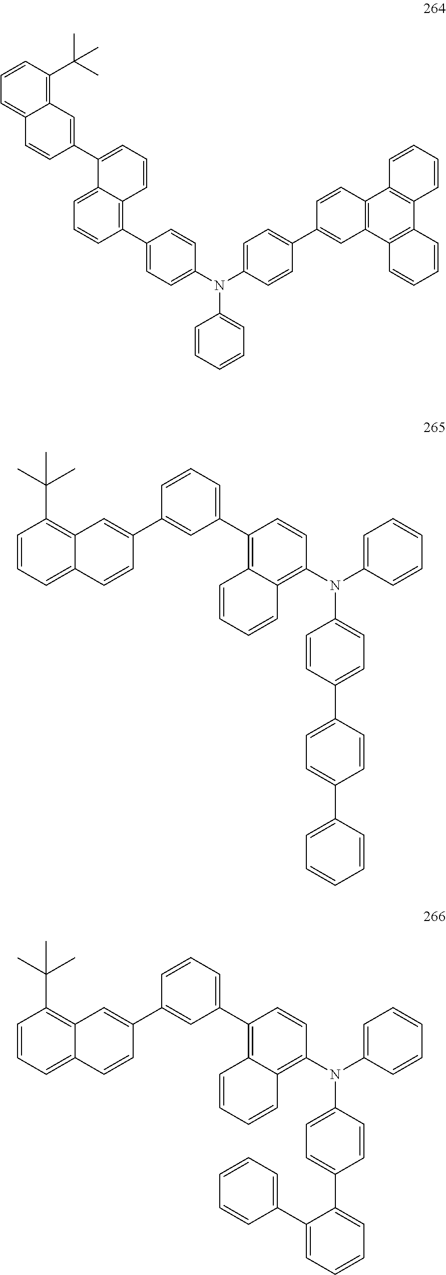 [SMILES: CC(C)(C)c1cccc2ccc(-c3cccc4c(-c5ccc(N(c6ccccc6)c6ccc(-c7ccc8c9ccccc9c9ccccc9c8c7)cc6)cc5)cccc34)cc12 ; CC(C)(C)c1cccc2ccc(-c3cccc(-c4ccc(N(c5ccccc5)c5ccc(-c6ccc(-c7ccccc7)cc6)cc5)c5ccccc45)c3)cc12 ; CC(C)(C)c1cccc2ccc(-c3cccc(-c4ccc(N(c5ccccc5)c5ccc(-c6ccccc6-c6ccccc6)cc5)c5ccccc45)c3)cc12]

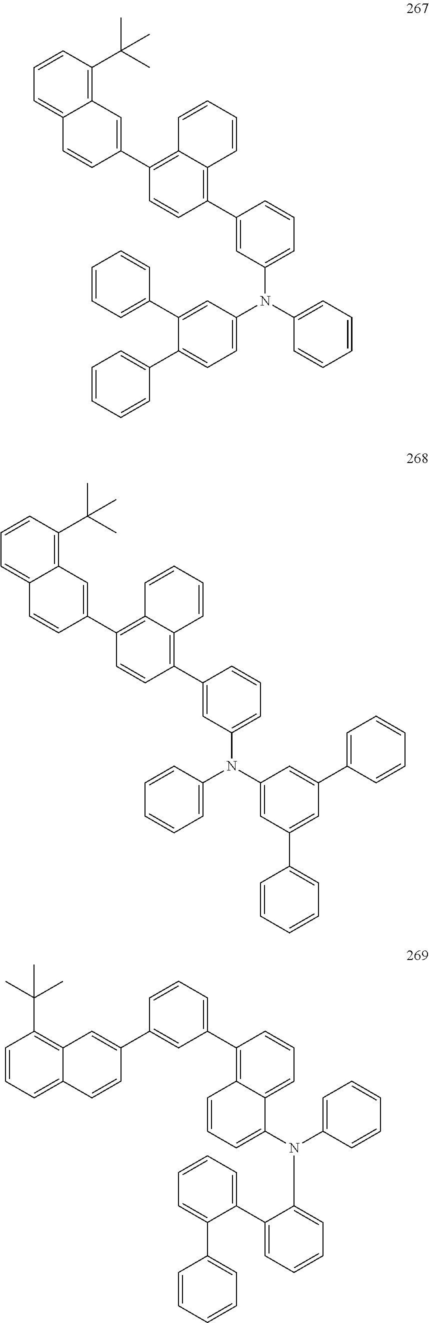 [SMILES: CC(C)(C)c1cccc2ccc(-c3ccc(-c4cccc(N(c5ccccc5)c5ccc(-c6ccccc6)c(-c6ccccc6)c5)c4)c4ccccc34)cc12 ; CC(C)(C)c1cccc2ccc(-c3ccc(-c4cccc(N(c5ccccc5)c5cc(-c6ccccc6)cc(-c6ccccc6)c5)c4)c4ccccc34)cc12 ; CC(C)(C)c1cccc2ccc(-c3cccc(-c4cccc5c(N(c6ccccc6)c6ccccc6-c6ccccc6-c6ccccc6)cccc45)c3)cc12]

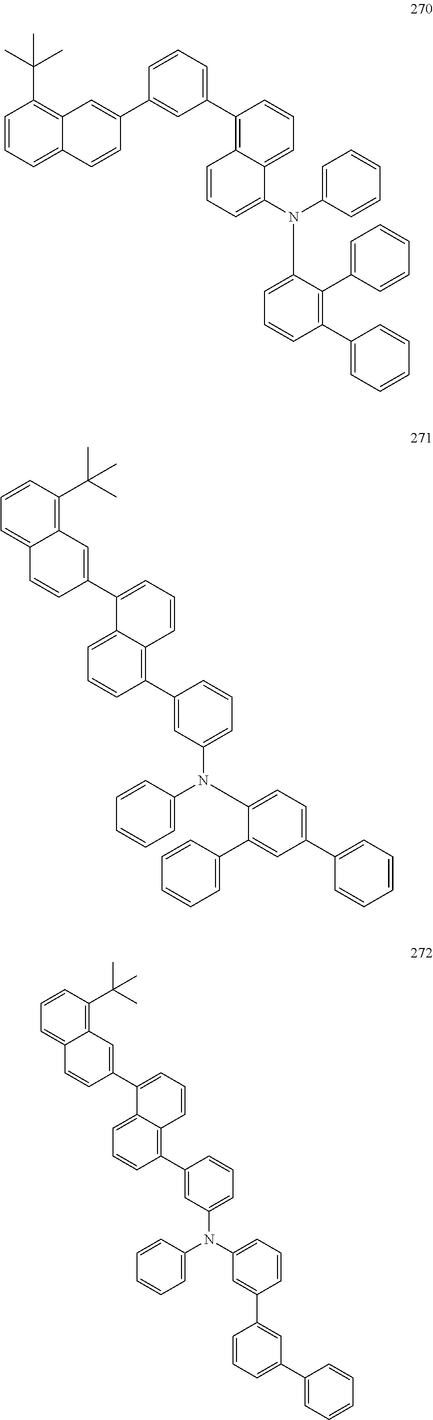 [SMILES: CC(C)(C)c1cccc2ccc(-c3cccc(-c4cccc5c(N(c6ccccc6)c6cccc(-c7ccccc7)c6-c6ccccc6)cccc45)c3)cc12 ; CC(C)(C)c1cccc2ccc(-c3cccc4c(-c5cccc(N(c6ccccc6)c6ccc(-c7ccccc7)cc6-c6ccccc6)c5)cccc34)cc12 ; CC(C)(C)c1cccc2ccc(-c3cccc4c(-c5cccc(N(c6ccccc6)c6cccc(-c7cccc(-c8ccccc8)c7)c6)c5)cccc34)cc12]

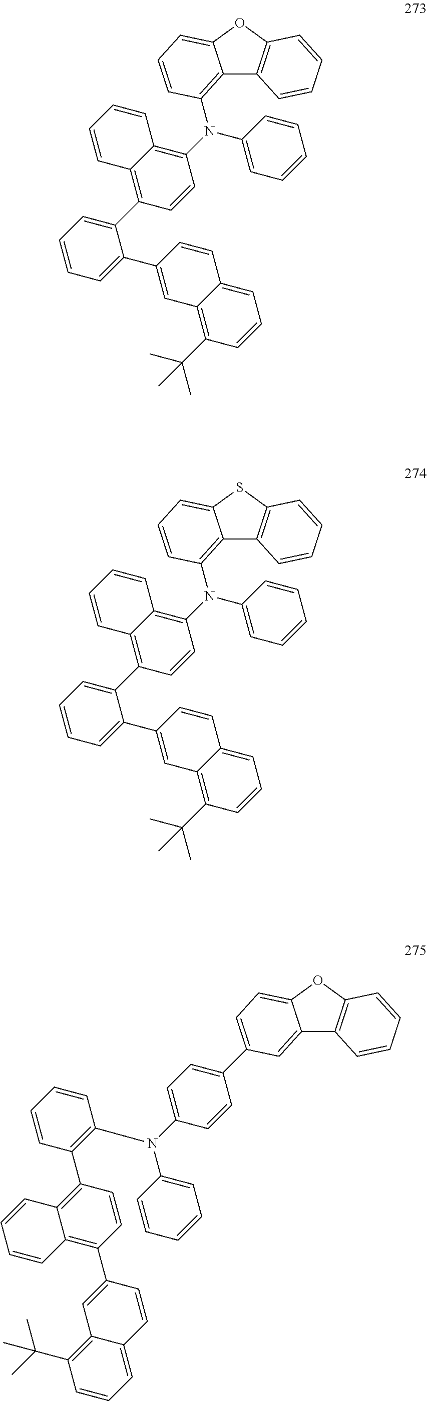 [SMILES: CC(C)(C)c1cccc2ccc(-c3ccccc3-c3ccc(N(c4ccccc4)c4cccc5oc6ccccc6c45)c4ccccc34)cc12 ; CC(C)(C)c1cccc2ccc(-c3ccccc3-c3ccc(N(c4ccccc4)c4cccc5sc6ccccc6c45)c4ccccc34)cc12 ; CC(C)(C)c1cccc2ccc(-c3ccc(-c4ccccc4N(c4ccccc4)c4ccc(-c5ccc6oc7ccccc7c6c5)cc4)c4ccccc34)cc12]

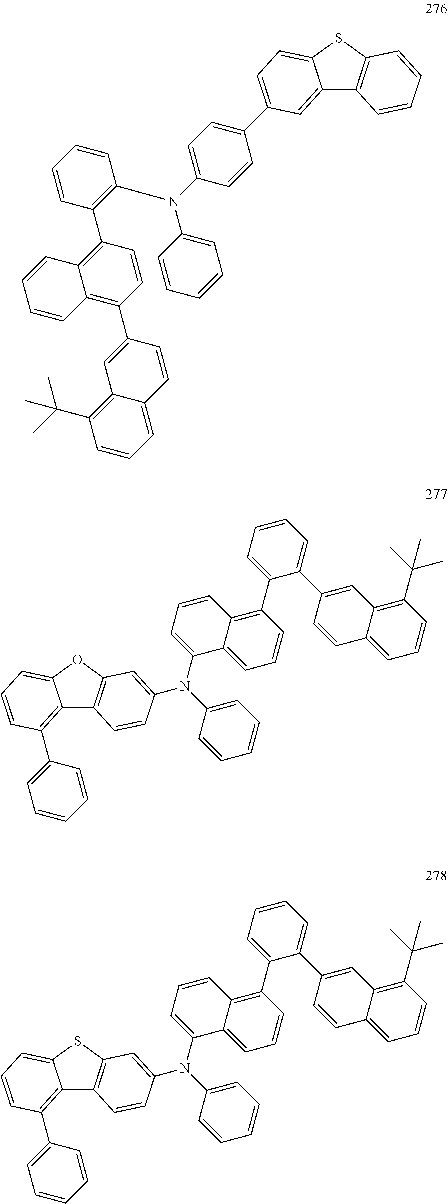 [SMILES: CC(C)(C)c1cccc2ccc(-c3ccc(-c4ccccc4N(c4ccccc4)c4ccc(-c5ccc6sc7ccccc7c6c5)cc4)c4ccccc34)cc12 ; CC(C)(C)c1cccc2ccc(-c3ccccc3-c3cccc4c(N(c5ccccc5)c5ccc6c(c5)oc5cccc(-c7ccccc7)c56)cccc34)cc12 ; CC(C)(C)c1cccc2ccc(-c3ccccc3-c3cccc4c(N(c5ccccc5)c5ccc6c(c5)sc5cccc(-c7ccccc7)c56)cccc34)cc12]

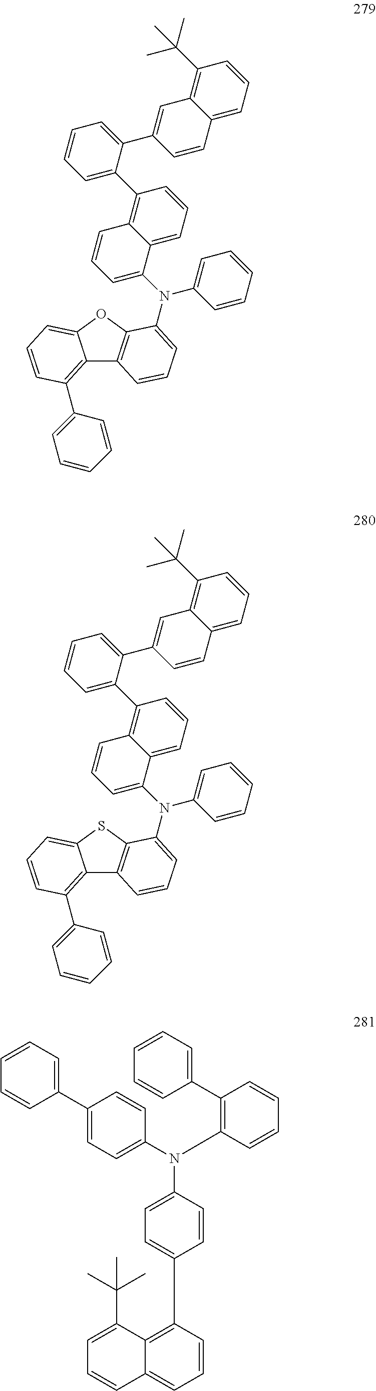 [SMILES: CC(C)(C)c1cccc2ccc(-c3ccccc3-c3cccc4c(N(c5ccccc5)c5cccc6c5oc5cccc(-c7ccccc7)c56)cccc34)cc12 ; CC(C)(C)c1cccc2ccc(-c3ccccc3-c3cccc4c(N(c5ccccc5)c5cccc6c5sc5cccc(-c7ccccc7)c56)cccc34)cc12 ; CC(C)(C)c1cccc2cccc(-c3ccc(N(c4ccc(-c5ccccc5)cc4)c4ccccc4-c4ccccc4)cc3)c12]

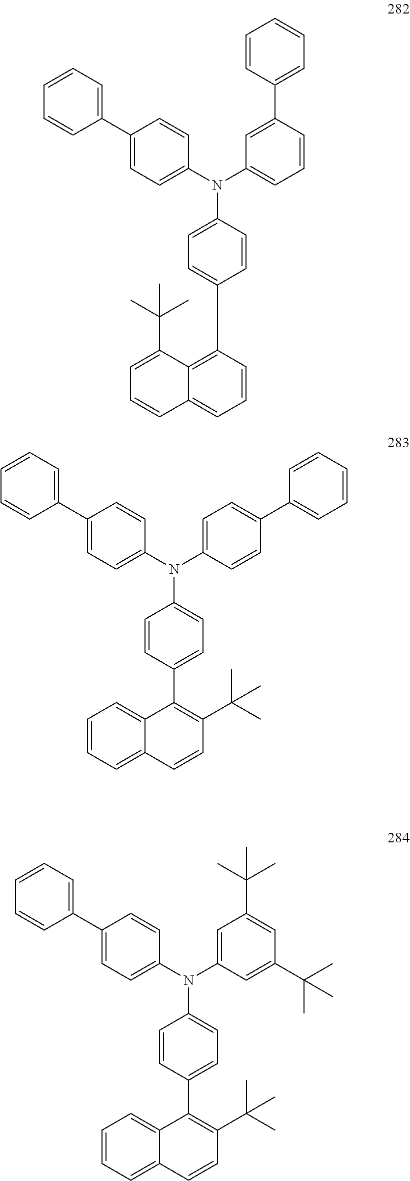 [SMILES: CC(C)(C)c1cccc2cccc(-c3ccc(N(c4ccc(-c5ccccc5)cc4)c4cccc(-c5ccccc5)c4)cc3)c12 ; CC(C)(C)c1ccc2ccccc2c1-c1ccc(N(c2ccc(-c3ccccc3)cc2)c2ccc(-c3ccccc3)cc2)cc1 ; CC(C)(C)c1cc(N(c2ccc(-c3ccccc3)cc2)c2ccc(-c3c(C(C)(C)C)ccc4ccccc34)cc2)cc(C(C)(C)C)c1]

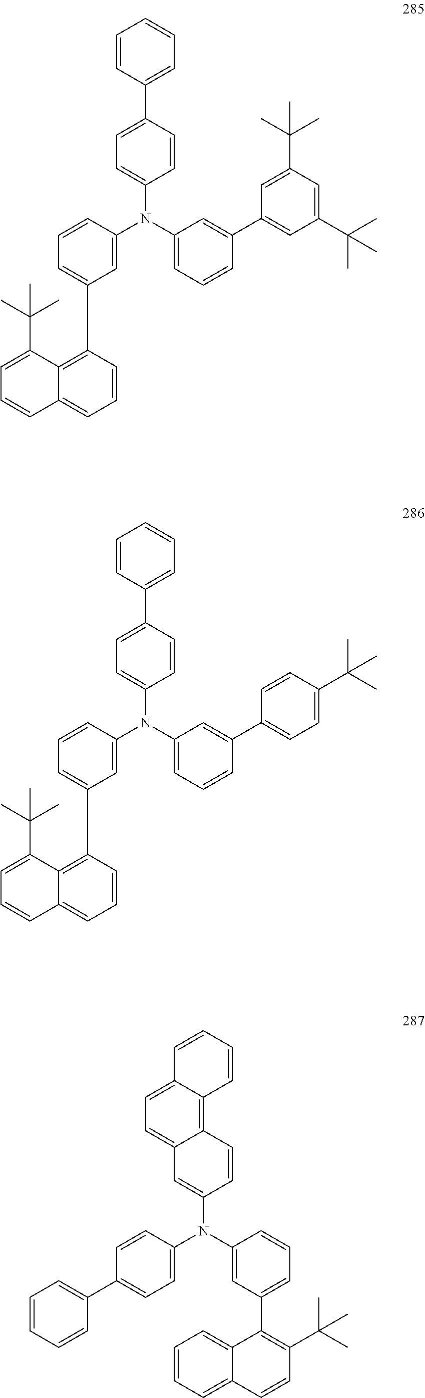 [SMILES: CC(C)(C)c1cc(-c2cccc(N(c3ccc(-c4ccccc4)cc3)c3cccc(-c4cccc5cccc(C(C)(C)C)c45)c3)c2)cc(C(C)(C)C)c1 ; CC(C)(C)c1ccc(-c2cccc(N(c3ccc(-c4ccccc4)cc3)c3cccc(-c4cccc5cccc(C(C)(C)C)c45)c3)c2)cc1 ; CC(C)(C)c1ccc2ccccc2c1-c1cccc(N(c2ccc(-c3ccccc3)cc2)c2ccc3c(ccc4ccccc43)c2)c1]

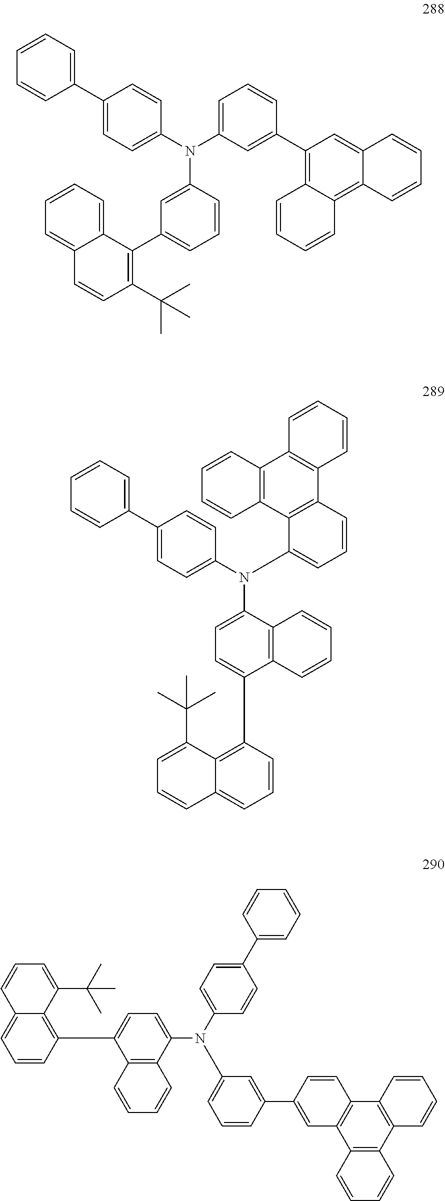 [SMILES: CC(C)(C)c1ccc2ccccc2c1-c1cccc(N(c2ccc(-c3ccccc3)cc2)c2cccc(-c3cc4ccccc4c4ccccc34)c2)c1 ; CC(C)(C)c1cccc2cccc(-c3ccc(N(c4ccc(-c5ccccc5)cc4)c4cccc5c6ccccc6c6ccccc6c45)c4ccccc34)c12 ; CC(C)(C)c1cccc2cccc(-c3ccc(N(c4ccc(-c5ccccc5)cc4)c4cccc(-c5ccc6c7ccccc7c7ccccc7c6c5)c4)c4ccccc34)c12]

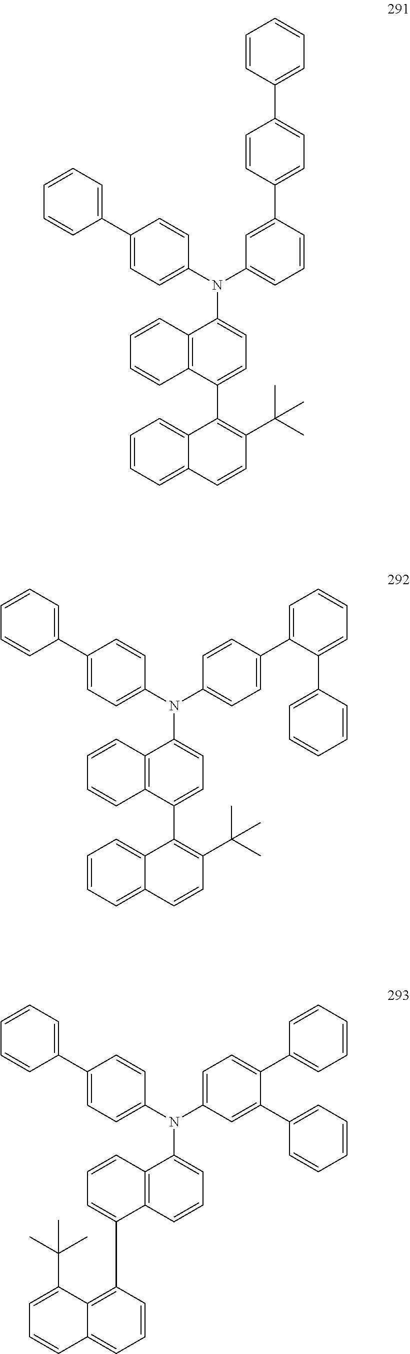 [SMILES: CC(C)(C)c1ccc2ccccc2c1-c1ccc(N(c2ccc(-c3ccccc3)cc2)c2cccc(-c3ccc(-c4ccccc4)cc3)c2)c2ccccc12 ; CC(C)(C)c1ccc2ccccc2c1-c1ccc(N(c2ccc(-c3ccccc3)cc2)c2ccc(-c3ccccc3-c3ccccc3)cc2)c2ccccc12 ; CC(C)(C)c1cccc2cccc(-c3cccc4c(N(c5ccc(-c6ccccc6)cc5)c5ccc(-c6ccccc6)c(-c6ccccc6)c5)cccc34)c12]

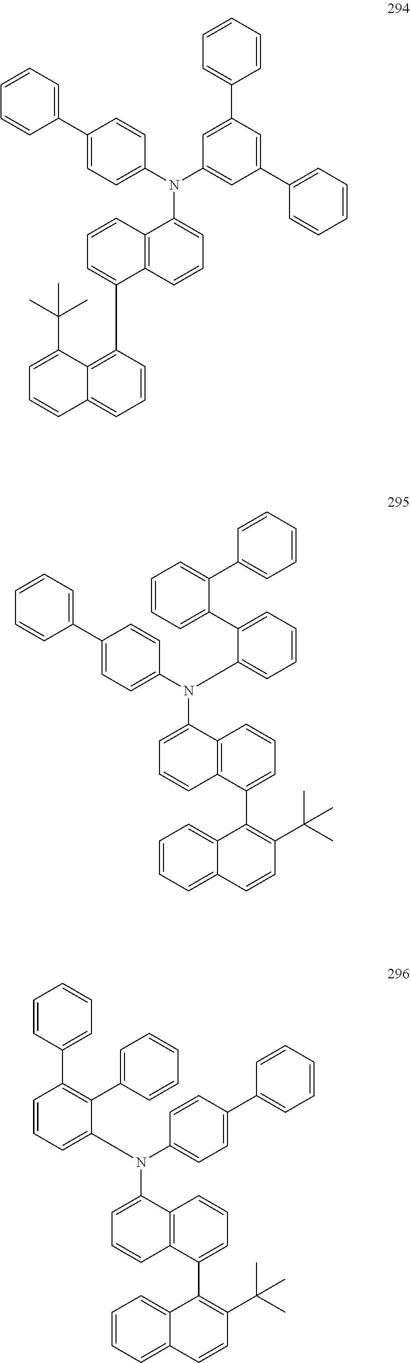 [SMILES: CC(C)(C)c1cccc2cccc(-c3cccc4c(N(c5ccc(-c6ccccc6)cc5)c5cc(-c6ccccc6)cc(-c6ccccc6)c5)cccc34)c12 ; CC(C)(C)c1ccc2ccccc2c1-c1cccc2c(N(c3ccc(-c4ccccc4)cc3)c3ccccc3-c3ccccc3-c3ccccc3)cccc12 ; CC(C)(C)c1ccc2ccccc2c1-c1cccc2c(N(c3ccc(-c4ccccc4)cc3)c3cccc(-c4ccccc4)c3-c3ccccc3)cccc12]

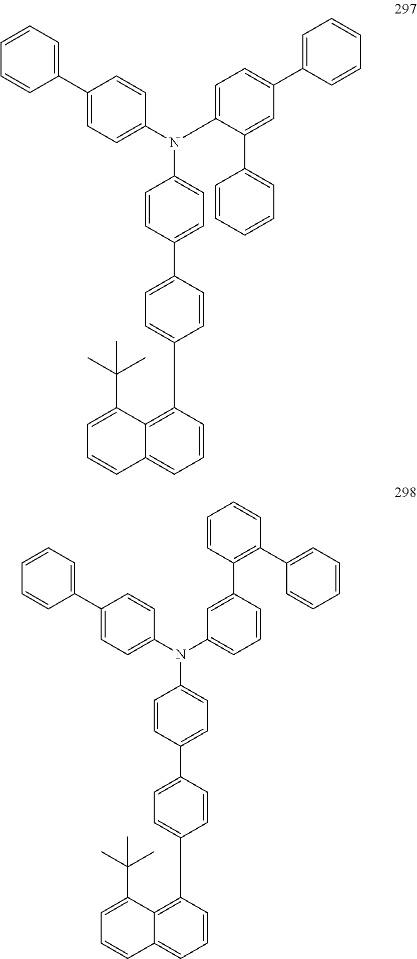 [SMILES: CC(C)(C)c1cccc2cccc(-c3ccc(-c4ccc(N(c5ccc(-c6ccccc6)cc5)c5ccc(-c6ccccc6)cc5-c5ccccc5)cc4)cc3)c12 ; CC(C)(C)c1cccc2cccc(-c3ccc(-c4ccc(N(c5ccc(-c6ccccc6)cc5)c5cccc(-c6ccccc6-c6ccccc6)c5)cc4)cc3)c12]

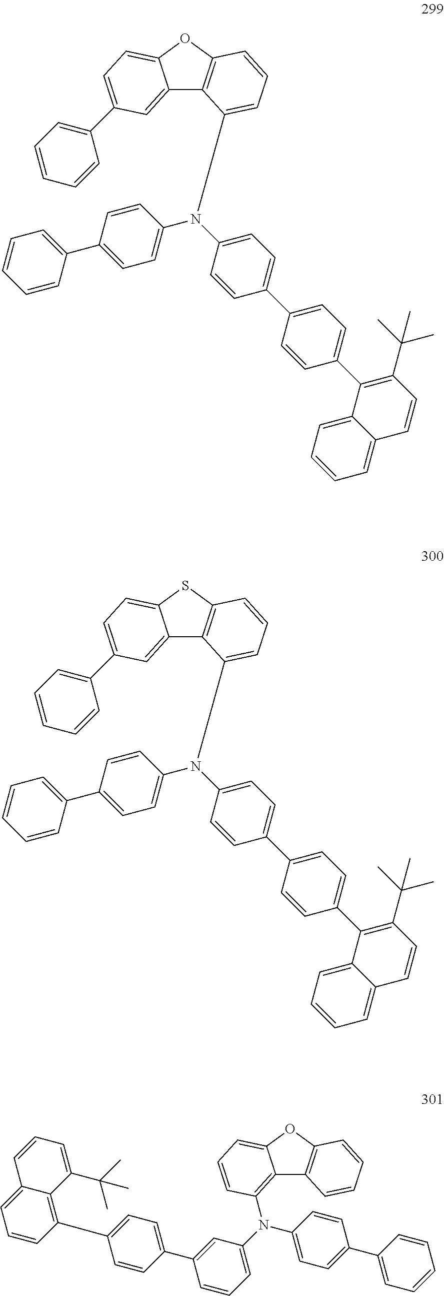 [SMILES: CC(C)(C)c1ccc2ccccc2c1-c1ccc(-c2ccc(N(c3ccc(-c4ccccc4)cc3)c3cccc4oc5ccc(-c6ccccc6)cc5c34)cc2)cc1 ; CC(C)(C)c1ccc2ccccc2c1-c1ccc(-c2ccc(N(c3ccc(-c4ccccc4)cc3)c3cccc4sc5ccc(-c6ccccc6)cc5c34)cc2)cc1 ; CC(C)(C)c1cccc2cccc(-c3ccc(-c4cccc(N(c5ccc(-c6ccccc6)cc5)c5cccc6oc7ccccc7c56)c4)cc3)c12]

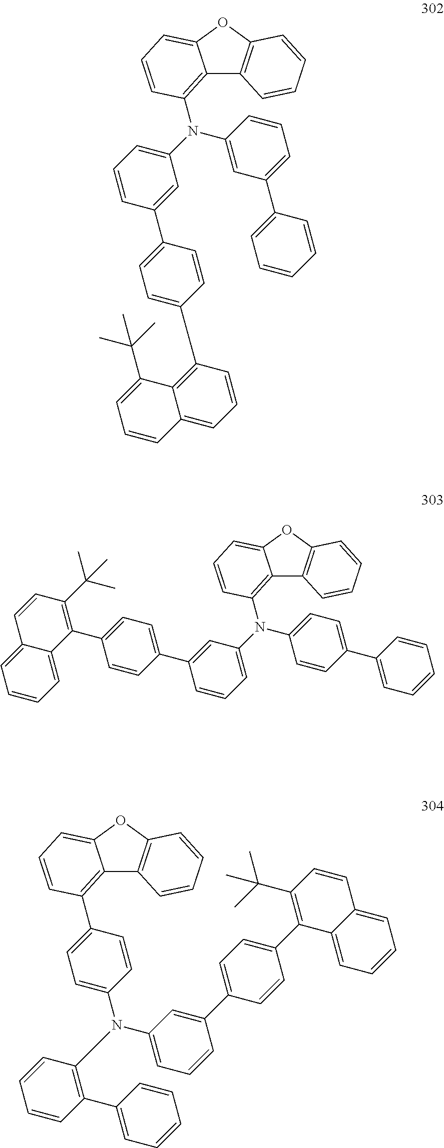 [SMILES: CC(C)(C)c1cccc2cccc(-c3ccc(-c4cccc(N(c5cccc(-c6ccccc6)c5)c5cccc6oc7ccccc7c56)c4)cc3)c12 ; CC(C)(C)c1ccc2ccccc2c1-c1ccc(-c2cccc(N(c3ccc(-c4ccccc4)cc3)c3cccc4oc5ccccc5c34)c2)cc1 ; CC(C)(C)c1ccc2ccccc2c1-c1ccc(-c2cccc(N(c3ccc(-c4cccc5oc6ccccc6c45)cc3)c3ccccc3-c3ccccc3)c2)cc1]

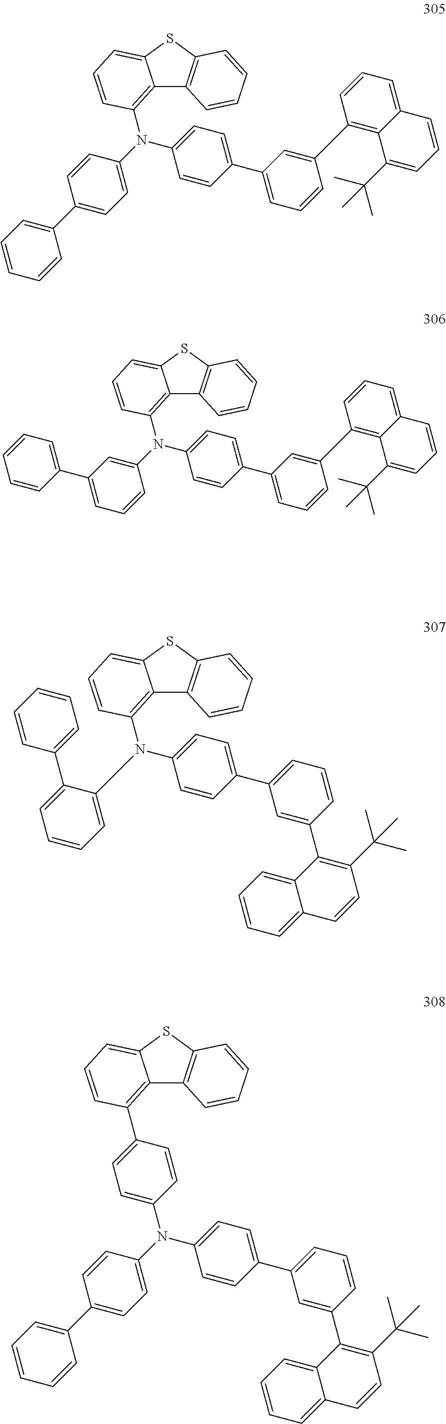 [SMILES: CC(C)(C)c1cccc2cccc(-c3cccc(-c4ccc(N(c5ccc(-c6ccccc6)cc5)c5cccc6sc7ccccc7c56)cc4)c3)c12 ; CC(C)(C)c1cccc2cccc(-c3cccc(-c4ccc(N(c5cccc(-c6ccccc6)c5)c5cccc6sc7ccccc7c56)cc4)c3)c12 ; CC(C)(C)c1ccc2ccccc2c1-c1cccc(-c2ccc(N(c3ccccc3-c3ccccc3)c3cccc4sc5ccccc5c34)cc2)c1 ; CC(C)(C)c1ccc2ccccc2c1-c1cccc(-c2ccc(N(c3ccc(-c4ccccc4)cc3)c3ccc(-c4cccc5sc6ccccc6c45)cc3)cc2)c1]

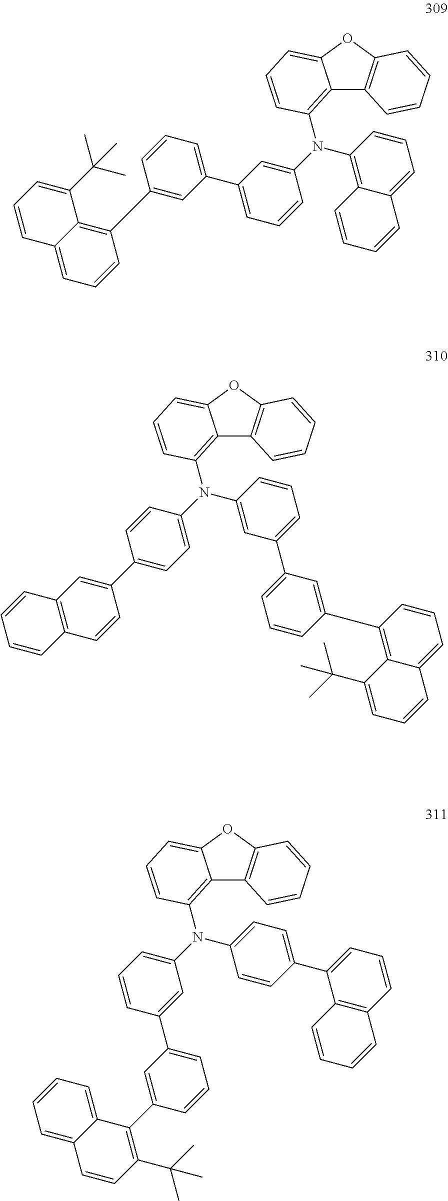 [SMILES: CC(C)(C)c1cccc2cccc(-c3cccc(-c4cccc(N(c5cccc6ccccc56)c5cccc6oc7ccccc7c56)c4)c3)c12 ; CC(C)(C)c1cccc2cccc(-c3cccc(-c4cccc(N(c5ccc(-c6ccc7ccccc7c6)cc5)c5cccc6oc7ccccc7c56)c4)c3)c12 ; CC(C)(C)c1ccc2ccccc2c1-c1cccc(-c2cccc(N(c3ccc(-c4cccc5ccccc45)cc3)c3cccc4oc5ccccc5c34)c2)c1]

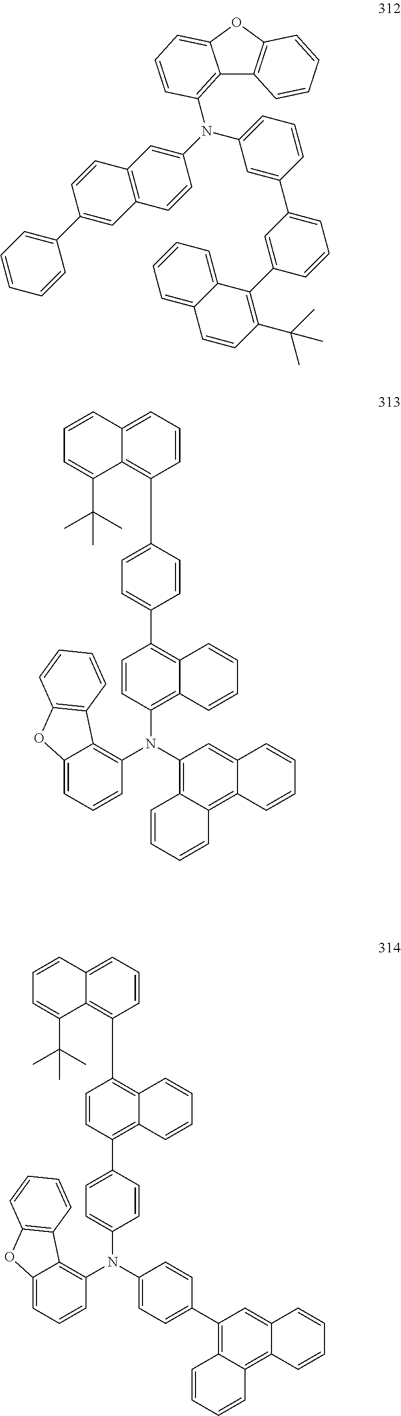 [SMILES: CC(C)(C)c1ccc2ccccc2c1-c1cccc(-c2cccc(N(c3ccc4cc(-c5ccccc5)ccc4c3)c3cccc4oc5ccccc5c34)c2)c1 ; CC(C)(C)c1cccc2cccc(-c3ccc(-c4ccc(N(c5cc6ccccc6c6ccccc56)c5cccc6oc7ccccc7c56)c5ccccc45)cc3)c12 ; CC(C)(C)c1cccc2cccc(-c3ccc(-c4ccc(N(c5ccc(-c6cc7ccccc7c7ccccc67)cc5)c5cccc6oc7ccccc7c56)cc4)c4ccccc34)c12]

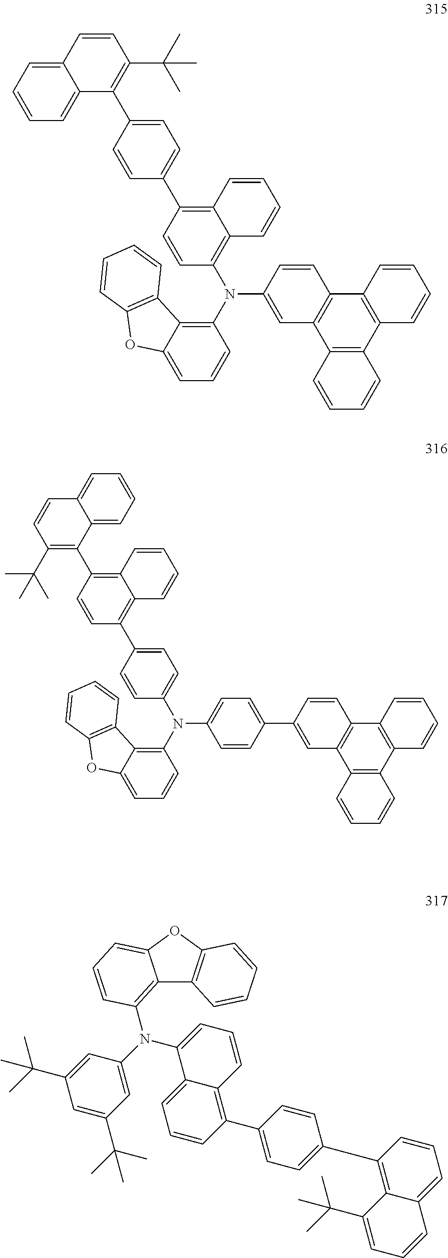 [SMILES: CC(C)(C)c1ccc2ccccc2c1-c1ccc(-c2ccc(N(c3ccc4c5ccccc5c5ccccc5c4c3)c3cccc4oc5ccccc5c34)c3ccccc23)cc1 ; CC(C)(C)c1ccc2ccccc2c1-c1ccc(-c2ccc(N(c3ccc(-c4ccc5c6ccccc6c6ccccc6c5c4)cc3)c3cccc4oc5ccccc5c34)cc2)c2ccccc12 ; CC(C)(C)c1cc(N(c2cccc3c(-c4ccc(-c5cccc6cccc(C(C)(C)C)c56)cc4)cccc23)c2cccc3oc4ccccc4c23)cc(C(C)(C)C)c1]

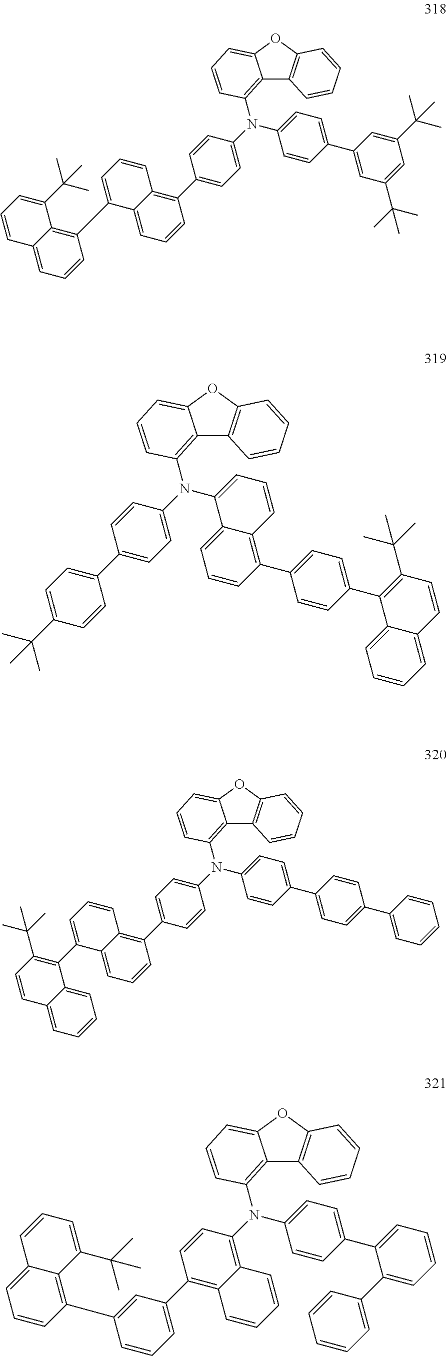

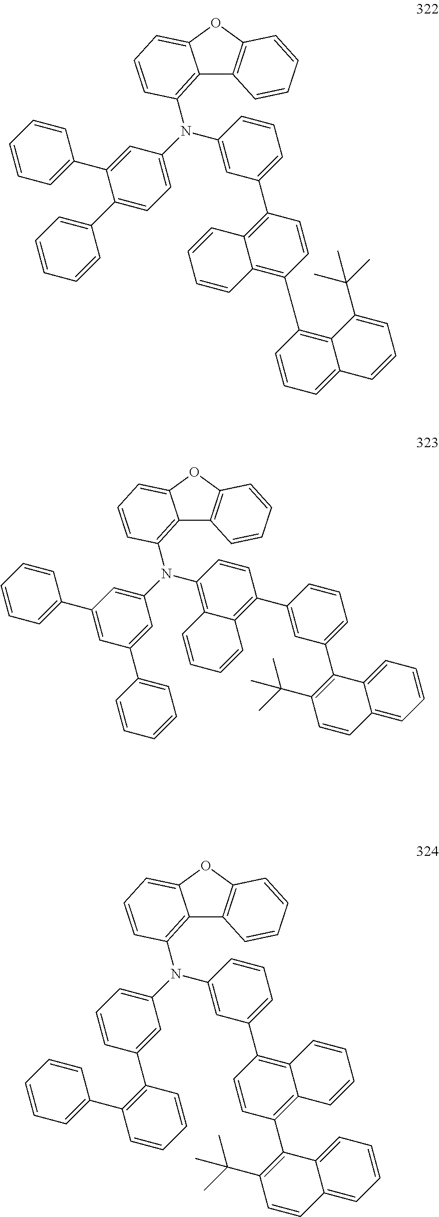 [SMILES: CC(C)(C)c1cccc2cccc(-c3ccc(-c4cccc(N(c5ccc(-c6ccccc6)c(-c6ccccc6)c5)c5cccc6oc7ccccc7c56)c4)c4ccccc34)c12 ; CC(C)(C)c1ccc2ccccc2c1-c1cccc(-c2ccc(N(c3cc(-c4ccccc4)cc(-c4ccccc4)c3)c3cccc4oc5ccccc5c34)c3ccccc23)c1 ; CC(C)(C)c1ccc2ccccc2c1-c1ccc(-c2cccc(N(c3cccc(-c4ccccc4-c4ccccc4)c3)c3cccc4oc5ccccc5c34)c2)c2ccccc12]

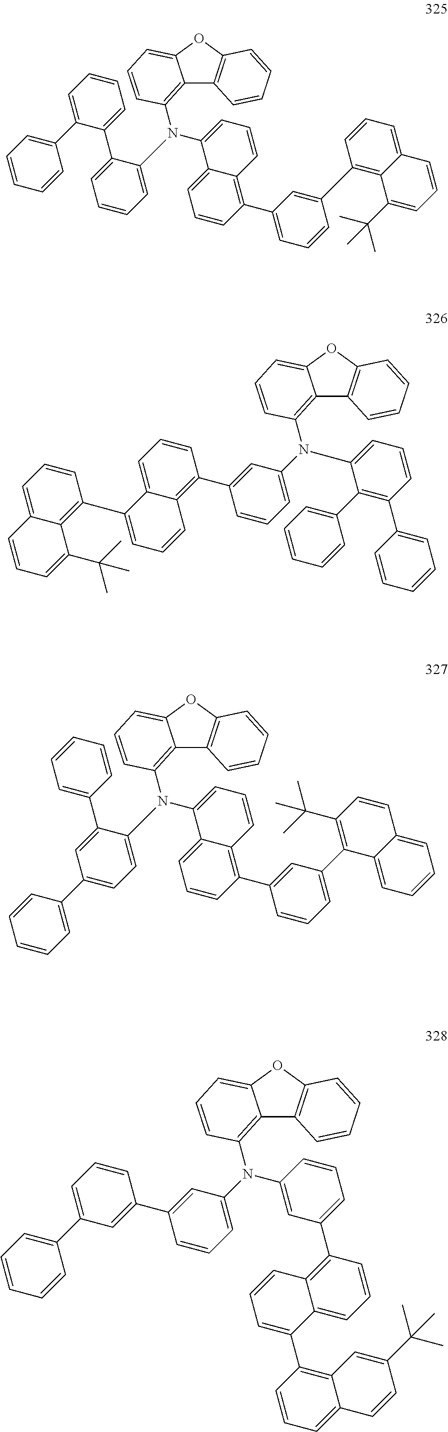 [SMILES: CC(C)(C)c1cccc2cccc(-c3cccc(-c4cccc5c(N(c6ccccc6-c6ccccc6-c6ccccc6)c6cccc7oc8ccccc8c67)cccc45)c3)c12 ; CC(C)(C)c1cccc2cccc(-c3cccc4c(-c5cccc(N(c6cccc(-c7ccccc7)c6-c6ccccc6)c6cccc7oc8ccccc8c67)c5)cccc34)c12 ; CC(C)(C)c1ccc2ccccc2c1-c1cccc(-c2cccc3c(N(c4ccc(-c5ccccc5)cc4-c4ccccc4)c4cccc5oc6ccccc6c45)cccc23)c1 ; CC(C)(C)c1ccc2cccc(-c3cccc4c(-c5cccc(N(c6cccc(-c7cccc(-c8ccccc8)c7)c6)c6cccc7oc8ccccc8c67)c5)cccc34)c2c1]

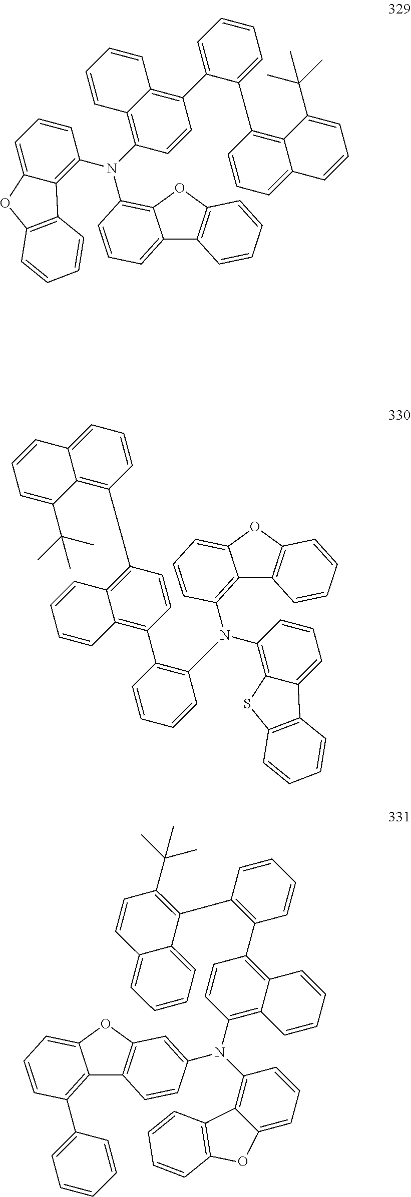 [SMILES: CC(C)(C)c1cccc2cccc(-c3ccccc3-c3ccc(N(c4cccc5c4oc4ccccc45)c4cccc5oc6ccccc6c45)c4ccccc34)c12 ; CC(C)(C)c1cccc2cccc(-c3ccc(-c4ccccc4N(c4cccc5c4sc4ccccc45)c4cccc5oc6ccccc6c45)c4ccccc34)c12 ; CC(C)(C)c1ccc2ccccc2c1-c1ccccc1-c1ccc(N(c2ccc3c(c2)oc2cccc(-c4ccccc4)c23)c2cccc3oc4ccccc4c23)c2ccccc12]

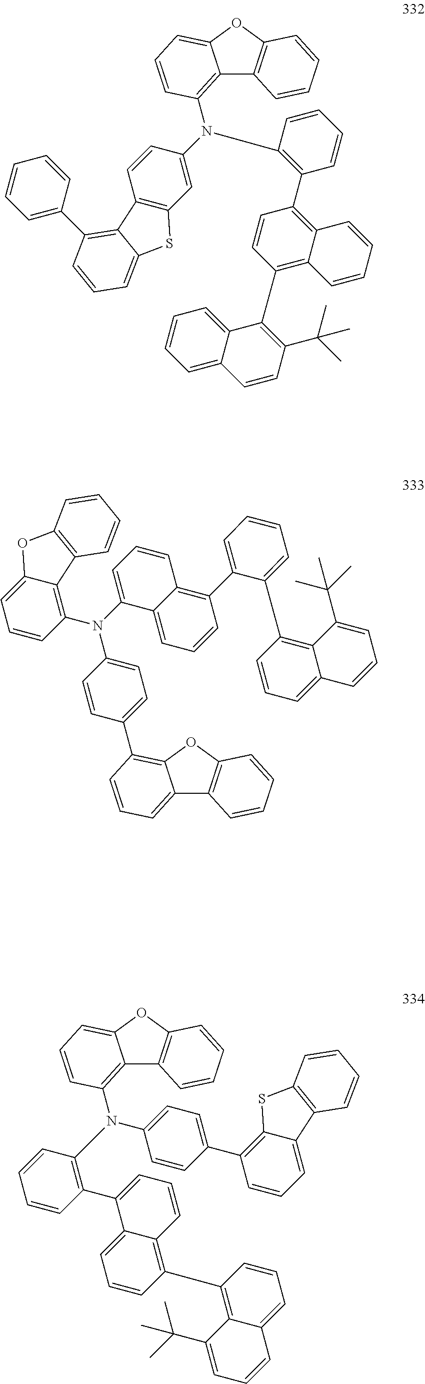 [SMILES: CC(C)(C)c1ccc2ccccc2c1-c1ccc(-c2ccccc2N(c2ccc3c(c2)sc2cccc(-c4ccccc4)c23)c2cccc3oc4ccccc4c23)c2ccccc12 ; CC(C)(C)c1cccc2cccc(-c3ccccc3-c3cccc4c(N(c5ccc(-c6cccc7c6oc6ccccc67)cc5)c5cccc6oc7ccccc7c56)cccc34)c12 ; CC(C)(C)c1cccc2cccc(-c3cccc4c(-c5ccccc5N(c5ccc(-c6cccc7c6sc6ccccc67)cc5)c5cccc6oc7ccccc7c56)cccc34)c12]

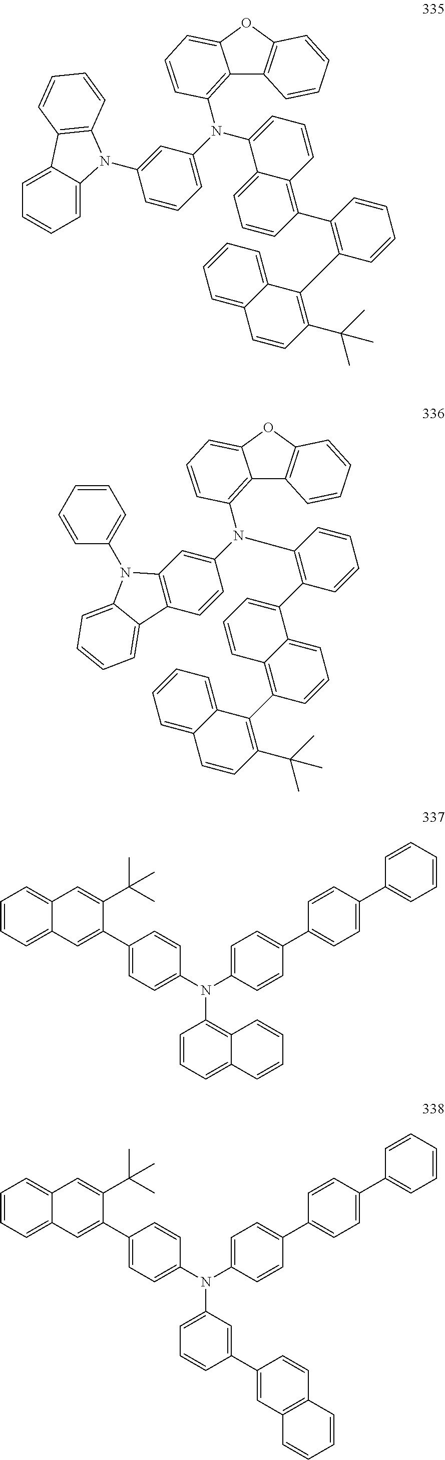 [SMILES: CC(C)(C)c1ccc2ccccc2c1-c1ccccc1-c1cccc2c(N(c3cccc(-n4c5ccccc5c5ccccc54)c3)c3cccc4oc5ccccc5c34)cccc12 ; CC(C)(C)c1ccc2ccccc2c1-c1cccc2c(-c3ccccc3N(c3ccc4c5ccccc5n(-c5ccccc5)c4c3)c3cccc4oc5ccccc5c34)cccc12 ; CC(C)(C)c1cc2ccccc2cc1-c1ccc(N(c2ccc(-c3ccc(-c4ccccc4)cc3)cc2)c2cccc3ccccc23)cc1 ; CC(C)(C)c1cc2ccccc2cc1-c1ccc(N(c2ccc(-c3ccc(-c4ccccc4)cc3)cc2)c2cccc(-c3ccc4ccccc4c3)c2)cc1]

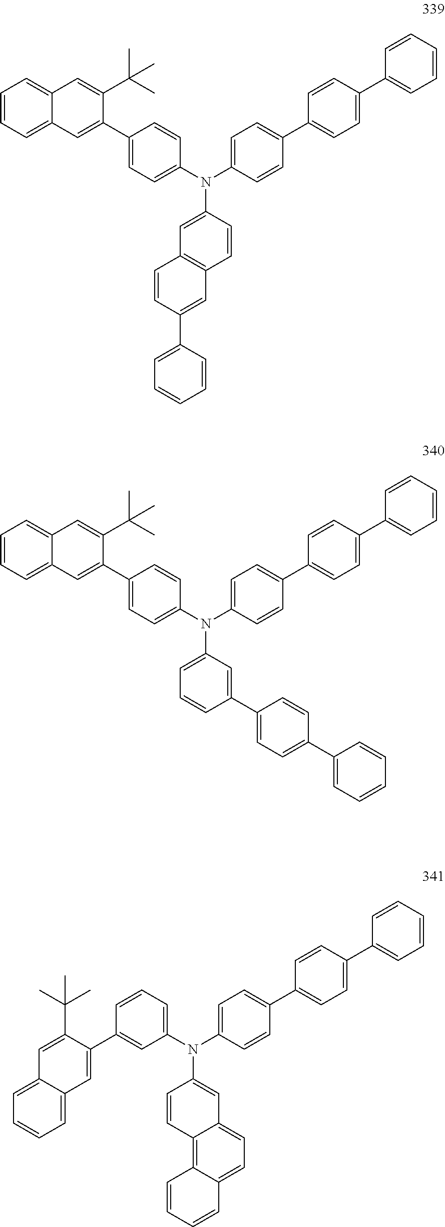 [SMILES: CC(C)(C)c1cc2ccccc2cc1-c1ccc(N(c2ccc(-c3ccc(-c4ccccc4)cc3)cc2)c2ccc3cc(-c4ccccc4)ccc3c2)cc1 ; CC(C)(C)c1cc2ccccc2cc1-c1ccc(N(c2ccc(-c3ccc(-c4ccccc4)cc3)cc2)c2cccc(-c3ccc(-c4ccccc4)cc3)c2)cc1 ; CC(C)(C)c1cc2ccccc2cc1-c1cccc(N(c2ccc(-c3ccc(-c4ccccc4)cc3)cc2)c2ccc3c(ccc4ccccc43)c2)c1]

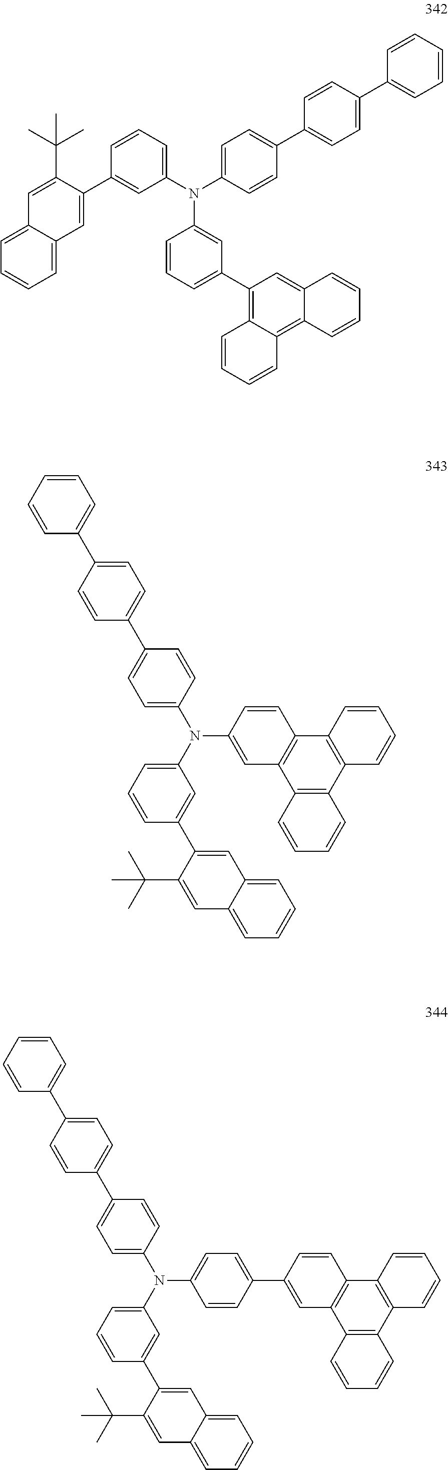 [SMILES: CC(C)(C)c1cc2ccccc2cc1-c1cccc(N(c2ccc(-c3ccc(-c4ccccc4)cc3)cc2)c2cccc(-c3cc4ccccc4c4ccccc34)c2)c1 ; CC(C)(C)c1cc2ccccc2cc1-c1cccc(N(c2ccc(-c3ccc(-c4ccccc4)cc3)cc2)c2ccc3c4ccccc4c4ccccc4c3c2)c1 ; CC(C)(C)c1cc2ccccc2cc1-c1cccc(N(c2ccc(-c3ccc(-c4ccccc4)cc3)cc2)c2ccc(-c3ccc4c5ccccc5c5ccccc5c4c3)cc2)c1]

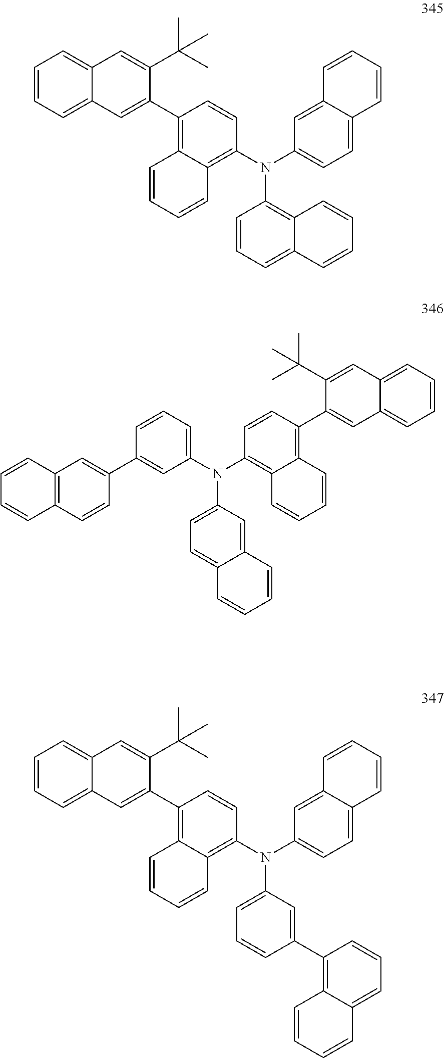 [SMILES: CC(C)(C)c1cc2ccccc2cc1-c1ccc(N(c2ccc3ccccc3c2)c2cccc3ccccc23)c2ccccc12 ; CC(C)(C)c1cc2ccccc2cc1-c1ccc(N(c2cccc(-c3ccc4ccccc4c3)c2)c2ccc3ccccc3c2)c2ccccc12 ; CC(C)(C)c1cc2ccccc2cc1-c1ccc(N(c2cccc(-c3cccc4ccccc34)c2)c2ccc3ccccc3c2)c2ccccc12]

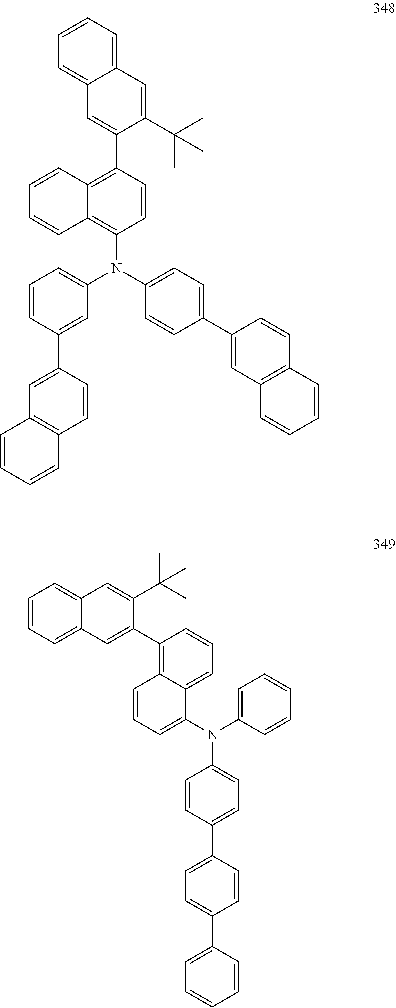 [SMILES: CC(C)(C)c1cc2ccccc2cc1-c1ccc(N(c2ccc(-c3ccc4ccccc4c3)cc2)c2cccc(-c3ccc4ccccc4c3)c2)c2ccccc12 ; CC(C)(C)c1cc2ccccc2cc1-c1cccc2c(N(c3ccccc3)c3ccc(-c4ccc(-c5ccccc5)cc4)cc3)cccc12]

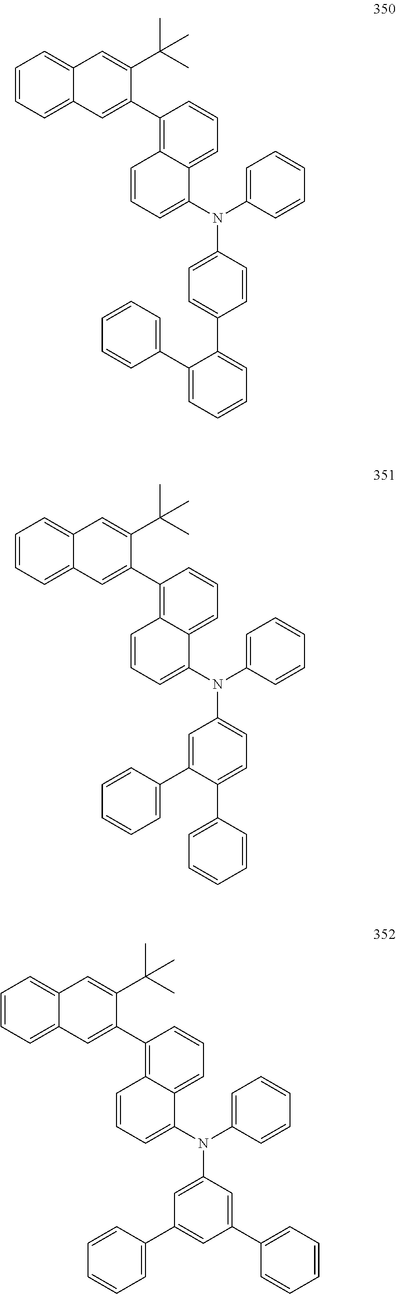 [SMILES: CC(C)(C)c1cc2ccccc2cc1-c1cccc2c(N(c3ccccc3)c3ccc(-c4ccccc4-c4ccccc4)cc3)cccc12 ; CC(C)(C)c1cc2ccccc2cc1-c1cccc2c(N(c3ccccc3)c3ccc(-c4ccccc4)c(-c4ccccc4)c3)cccc12 ; CC(C)(C)c1cc2ccccc2cc1-c1cccc2c(N(c3ccccc3)c3cc(-c4ccccc4)cc(-c4ccccc4)c3)cccc12]

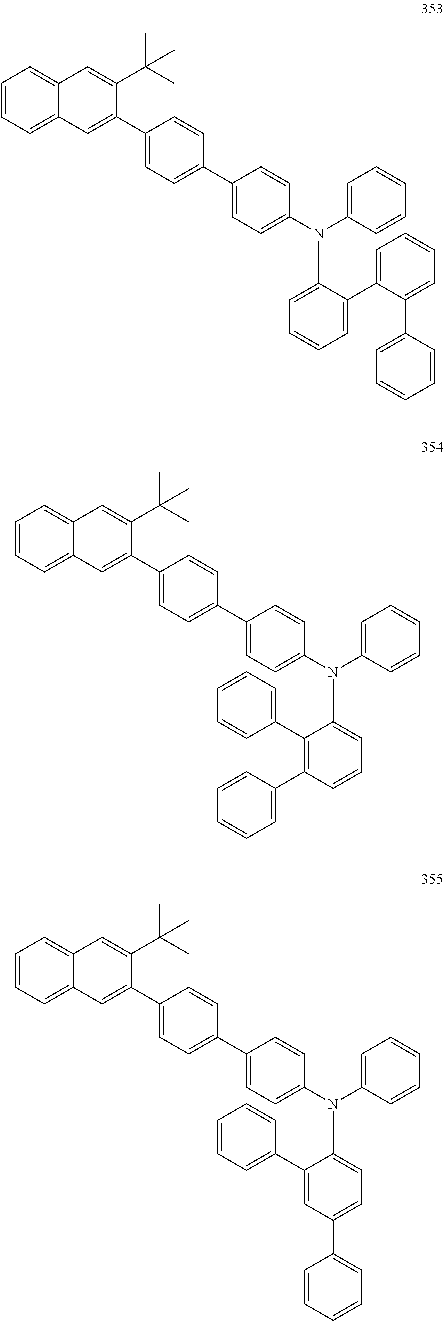 [SMILES: CC(C)(C)c1cc2ccccc2cc1-c1ccc(-c2ccc(N(c3ccccc3)c3ccccc3-c3ccccc3-c3ccccc3)cc2)cc1 ; CC(C)(C)c1cc2ccccc2cc1-c1ccc(-c2ccc(N(c3ccccc3)c3cccc(-c4ccccc4)c3-c3ccccc3)cc2)cc1 ; CC(C)(C)c1cc2ccccc2cc1-c1ccc(-c2ccc(N(c3ccccc3)c3ccc(-c4ccccc4)cc3-c3ccccc3)cc2)cc1]

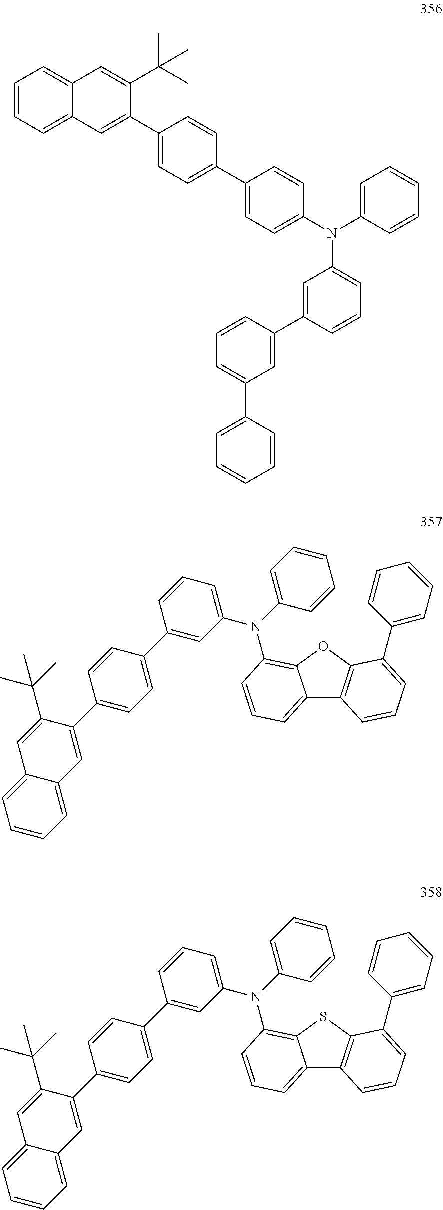 [SMILES: CC(C)(C)c1cc2ccccc2cc1-c1ccc(-c2ccc(N(c3ccccc3)c3cccc(-c4cccc(-c5ccccc5)c4)c3)cc2)cc1 ; CC(C)(C)c1cc2ccccc2cc1-c1ccc(-c2cccc(N(c3ccccc3)c3cccc4c3oc3c(-c5ccccc5)cccc34)c2)cc1 ; CC(C)(C)c1cc2ccccc2cc1-c1ccc(-c2cccc(N(c3ccccc3)c3cccc4c3sc3c(-c5ccccc5)cccc34)c2)cc1]

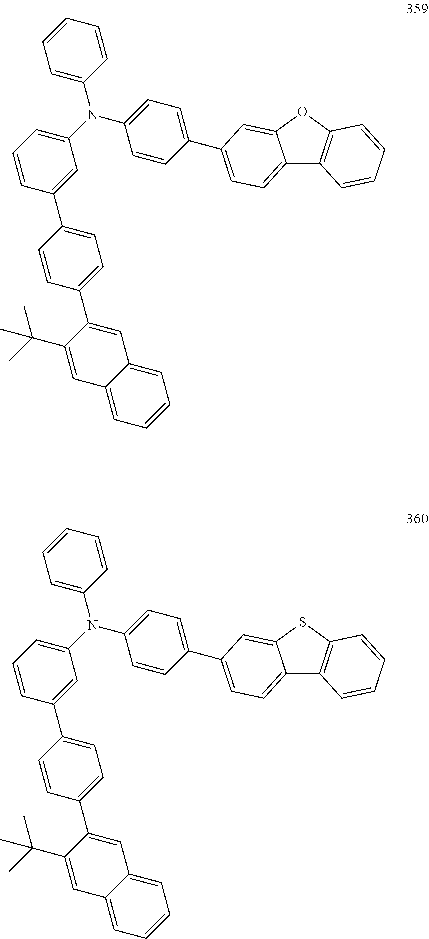 [SMILES: CC(C)(C)c1cc2ccccc2cc1-c1ccc(-c2cccc(N(c3ccccc3)c3ccc(-c4ccc5c(c4)oc4ccccc45)cc3)c2)cc1 ; CC(C)(C)c1cc2ccccc2cc1-c1ccc(-c2cccc(N(c3ccccc3)c3ccc(-c4ccc5c(c4)sc4ccccc45)cc3)c2)cc1]

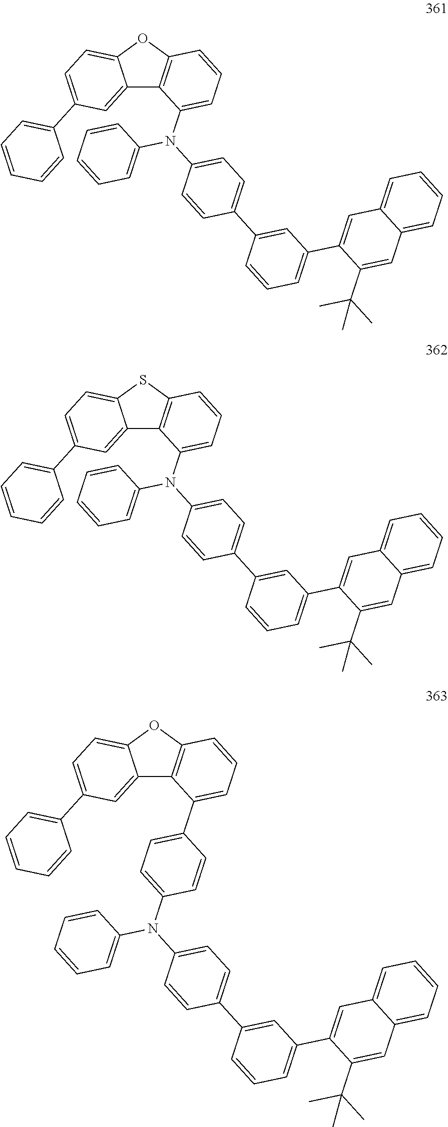 [SMILES: CC(C)(C)c1cc2ccccc2cc1-c1cccc(-c2ccc(N(c3ccccc3)c3cccc4oc5ccc(-c6ccccc6)cc5c34)cc2)c1 ; CC(C)(C)c1cc2ccccc2cc1-c1cccc(-c2ccc(N(c3ccccc3)c3cccc4sc5ccc(-c6ccccc6)cc5c34)cc2)c1 ; CC(C)(C)c1cc2ccccc2cc1-c1cccc(-c2ccc(N(c3ccccc3)c3ccc(-c4cccc5oc6ccc(-c7ccccc7)cc6c45)cc3)cc2)c1]

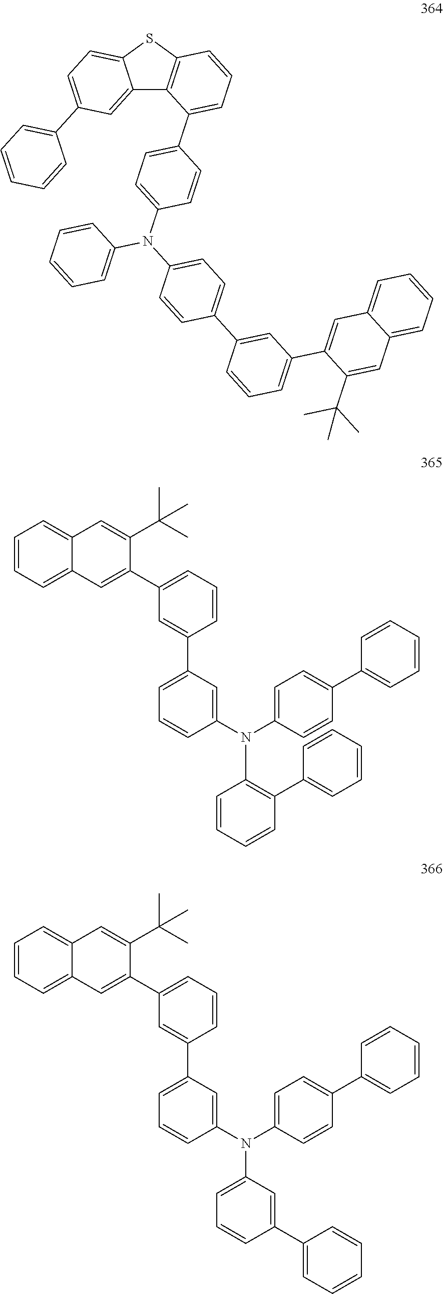 [SMILES: CC(C)(C)c1cc2ccccc2cc1-c1cccc(-c2ccc(N(c3ccccc3)c3ccc(-c4cccc5sc6ccc(-c7ccccc7)cc6c45)cc3)cc2)c1 ; CC(C)(C)c1cc2ccccc2cc1-c1cccc(-c2cccc(N(c3ccc(-c4ccccc4)cc3)c3ccccc3-c3ccccc3)c2)c1 ; CC(C)(C)c1cc2ccccc2cc1-c1cccc(-c2cccc(N(c3ccc(-c4ccccc4)cc3)c3cccc(-c4ccccc4)c3)c2)c1]

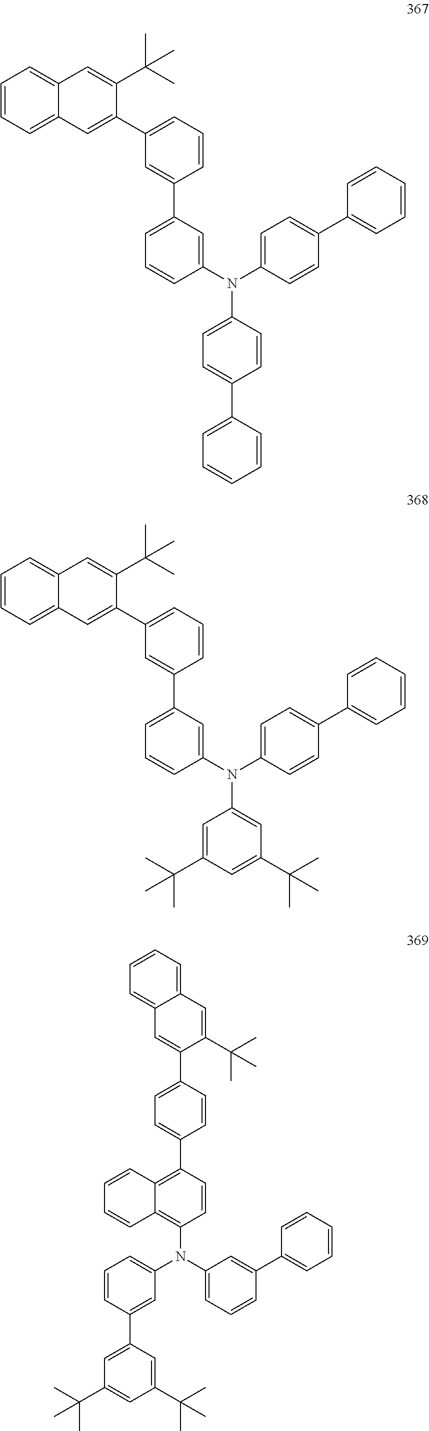

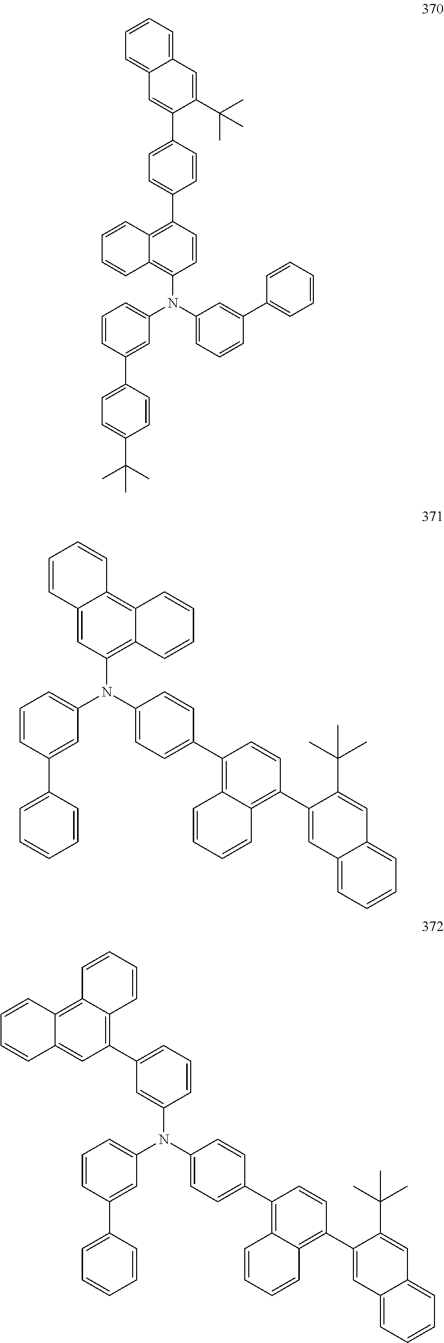 [SMILES: CC(C)(C)c1ccc(-c2cccc(N(c3cccc(-c4ccccc4)c3)c3ccc(-c4ccc(-c5cc6ccccc6cc5C(C)(C)C)cc4)c4ccccc34)c2)cc1 ; CC(C)(C)c1cc2ccccc2cc1-c1ccc(-c2ccc(N(c3cccc(-c4ccccc4)c3)c3cc4ccccc4c4ccccc34)cc2)c2ccccc12 ; CC(C)(C)c1cc2ccccc2cc1-c1ccc(-c2ccc(N(c3cccc(-c4ccccc4)c3)c3cccc(-c4cc5ccccc5c5ccccc45)c3)cc2)c2ccccc12]

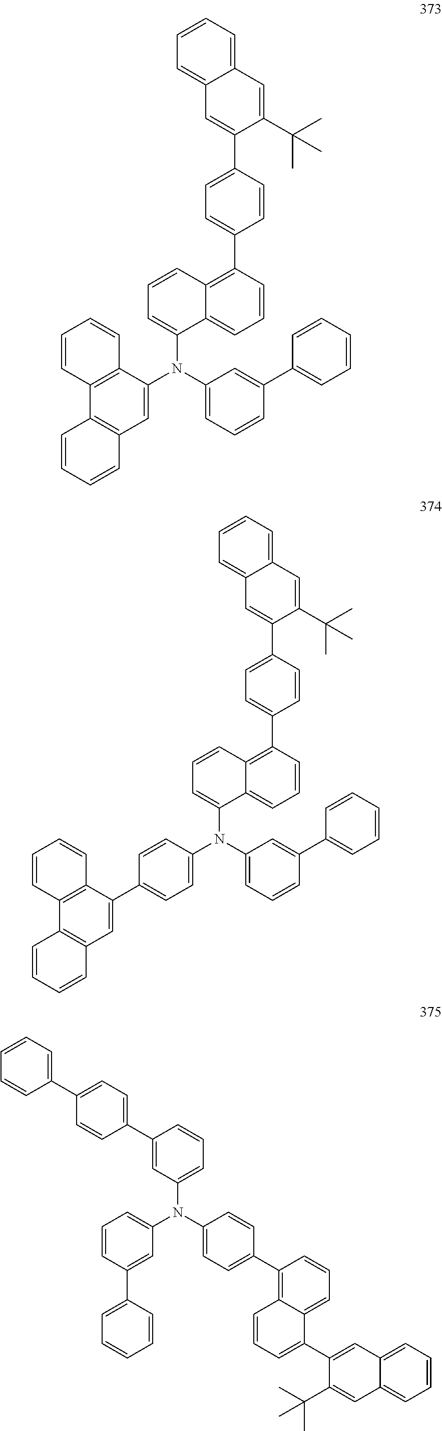 [SMILES: CC(C)(C)c1cc2ccccc2cc1-c1ccc(-c2cccc3c(N(c4cccc(-c5ccccc5)c4)c4cc5ccccc5c5ccccc45)cccc23)cc1 ; CC(C)(C)c1cc2ccccc2cc1-c1ccc(-c2cccc3c(N(c4ccc(-c5cc6ccccc6c6ccccc56)cc4)c4cccc(-c5ccccc5)c4)cccc23)cc1 ; CC(C)(C)c1cc2ccccc2cc1-c1cccc2c(-c3ccc(N(c4cccc(-c5ccccc5)c4)c4cccc(-c5ccc(-c6ccccc6)cc5)c4)cc3)cccc12]

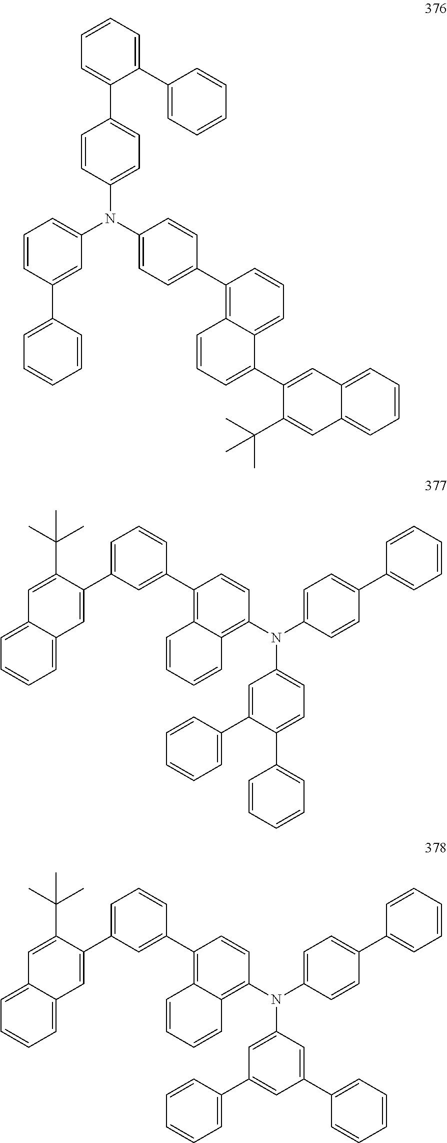 [SMILES: CC(C)(C)c1cc2ccccc2cc1-c1cccc2c(-c3ccc(N(c4ccc(-c5ccccc5-c5ccccc5)cc4)c4cccc(-c5ccccc5)c4)cc3)cccc12 ; CC(C)(C)c1cc2ccccc2cc1-c1cccc(-c2ccc(N(c3ccc(-c4ccccc4)cc3)c3ccc(-c4ccccc4)c(-c4ccccc4)c3)c3ccccc23)c1 ; CC(C)(C)c1cc2ccccc2cc1-c1cccc(-c2ccc(N(c3ccc(-c4ccccc4)cc3)c3cc(-c4ccccc4)cc(-c4ccccc4)c3)c3ccccc23)c1]

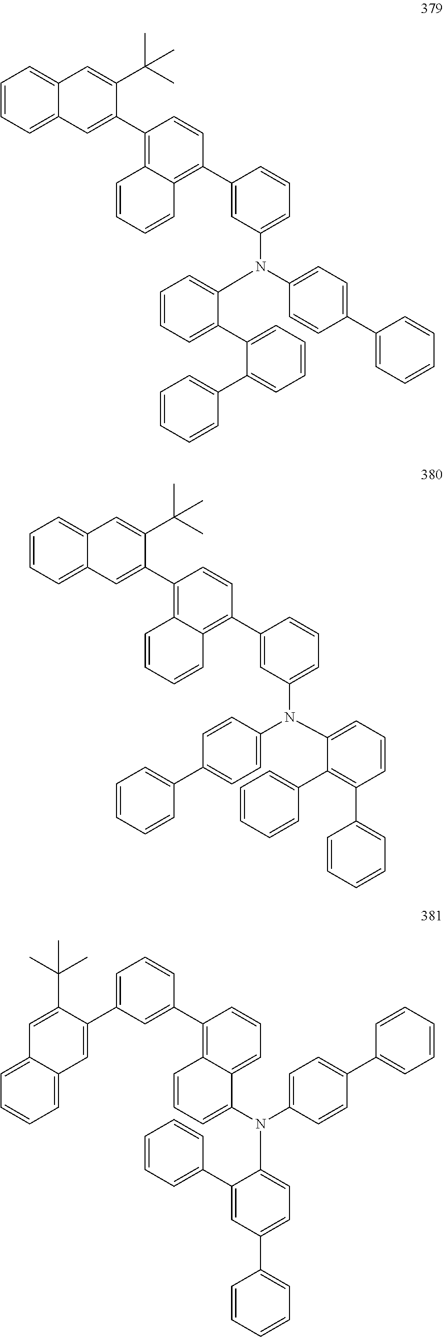

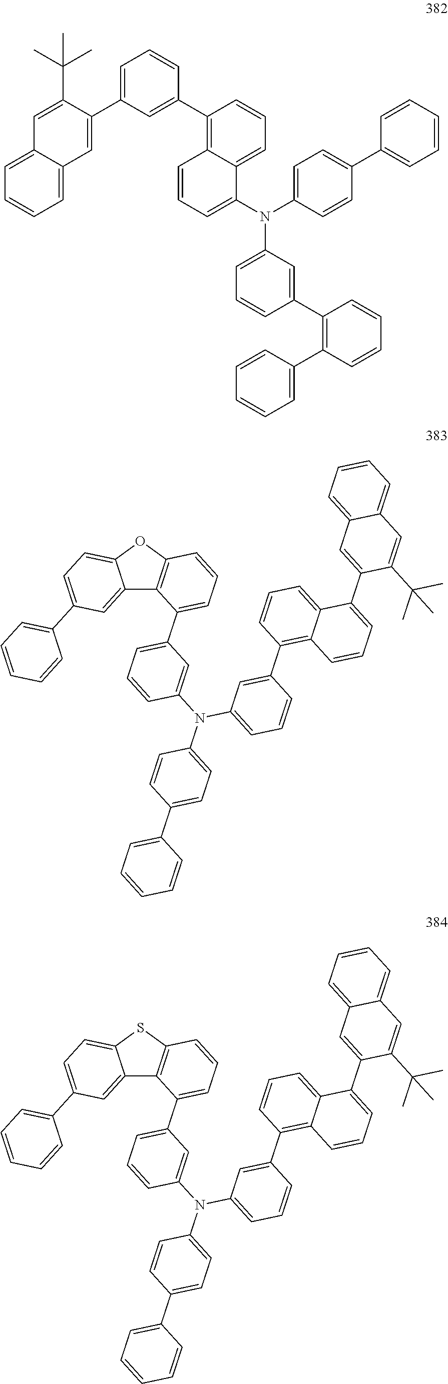 [SMILES: CC(C)(C)c1cc2ccccc2cc1-c1cccc(-c2cccc3c(N(c4ccc(-c5ccccc5)cc4)c4cccc(-c5ccccc5-c5ccccc5)c4)cccc23)c1 ; CC(C)(C)c1cc2ccccc2cc1-c1cccc2c(-c3cccc(N(c4ccc(-c5ccccc5)cc4)c4cccc(-c5cccc6oc7ccc(-c8ccccc8)cc7c56)c4)c3)cccc12 ; CC(C)(C)c1cc2ccccc2cc1-c1cccc2c(-c3cccc(N(c4ccc(-c5ccccc5)cc4)c4cccc(-c5cccc6sc7ccc(-c8ccccc8)cc7c56)c4)c3)cccc12]

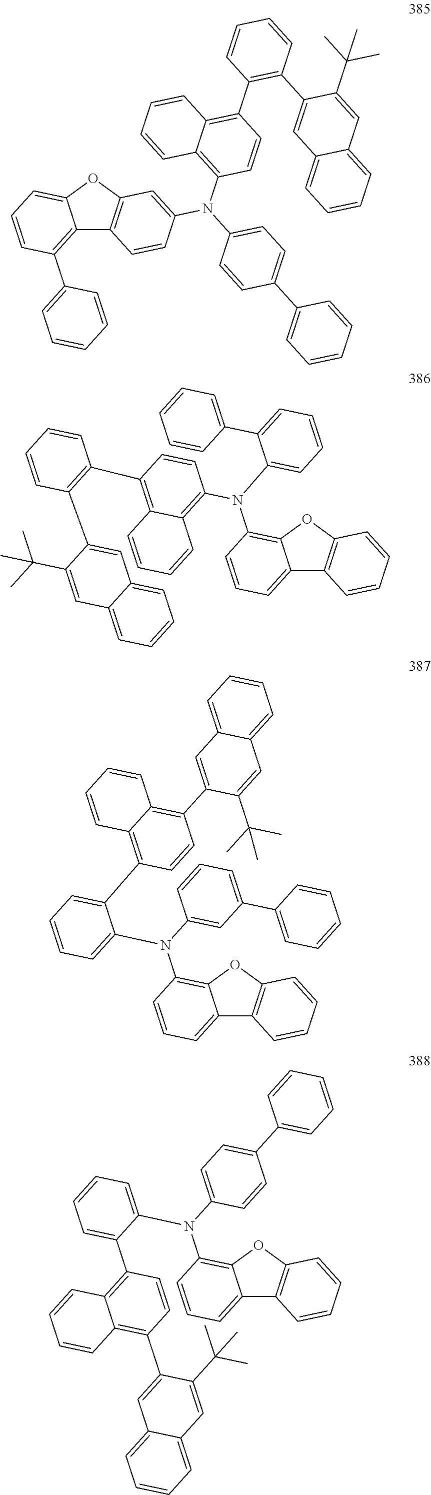

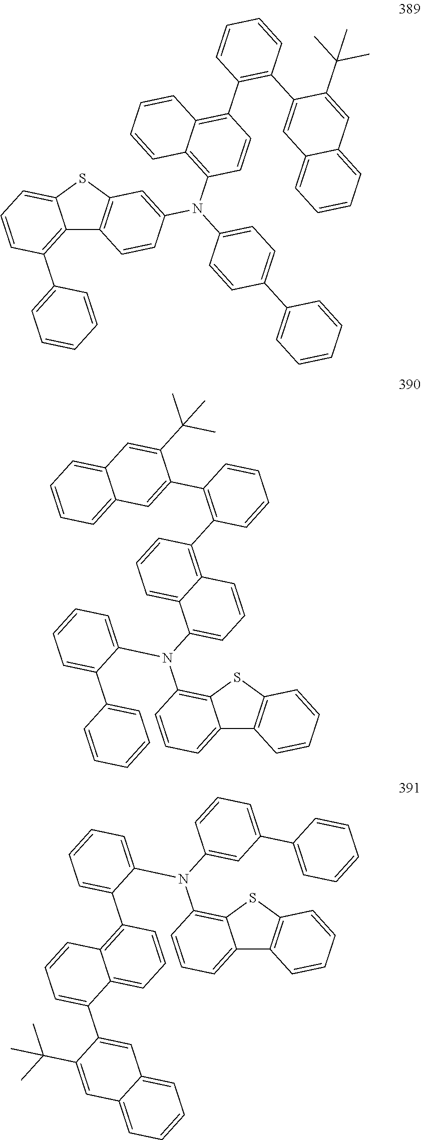

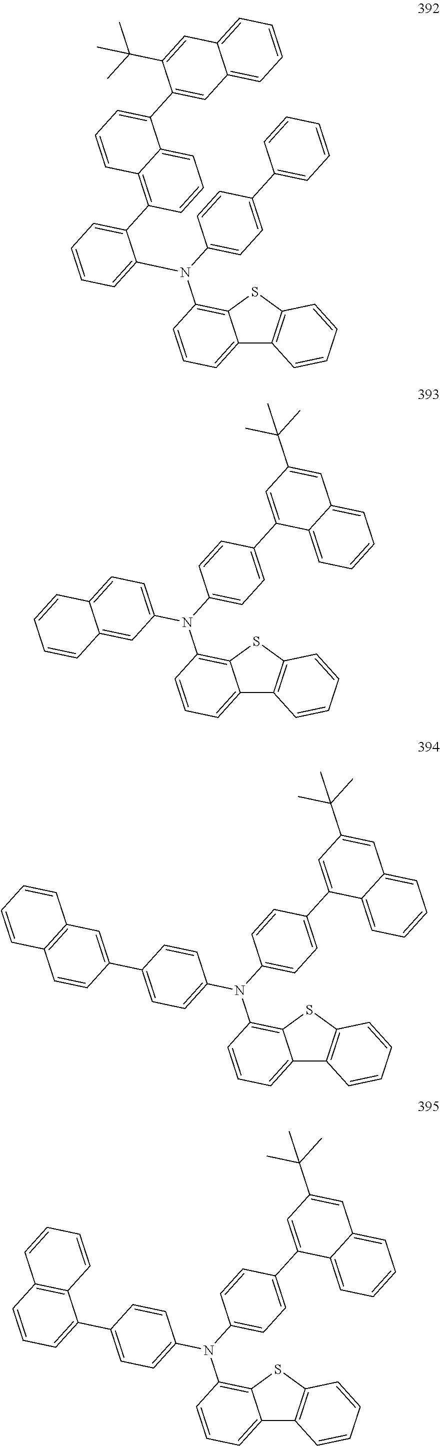

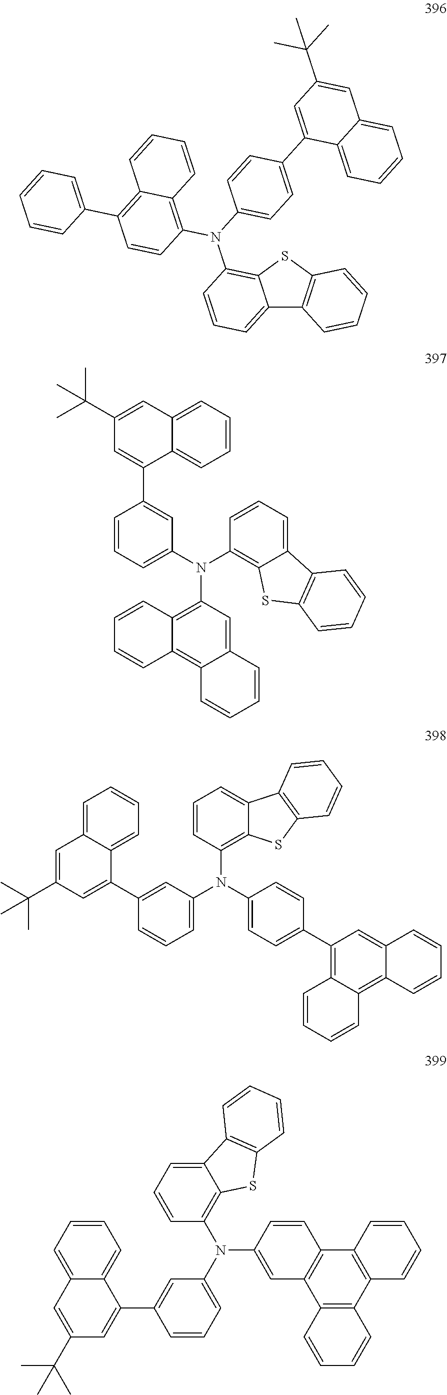 [SMILES: CC(C)(C)c1cc(-c2ccc(N(c3ccc(-c4ccccc4)c4ccccc34)c3cccc4c3sc3ccccc34)cc2)c2ccccc2c1 ; CC(C)(C)c1cc(-c2cccc(N(c3cc4ccccc4c4ccccc34)c3cccc4c3sc3ccccc34)c2)c2ccccc2c1 ; CC(C)(C)c1cc(-c2cccc(N(c3ccc(-c4cc5ccccc5c5ccccc45)cc3)c3cccc4c3sc3ccccc34)c2)c2ccccc2c1 ; CC(C)(C)c1cc(-c2cccc(N(c3ccc4c5ccccc5c5ccccc5c4c3)c3cccc4c3sc3ccccc34)c2)c2ccccc2c1]

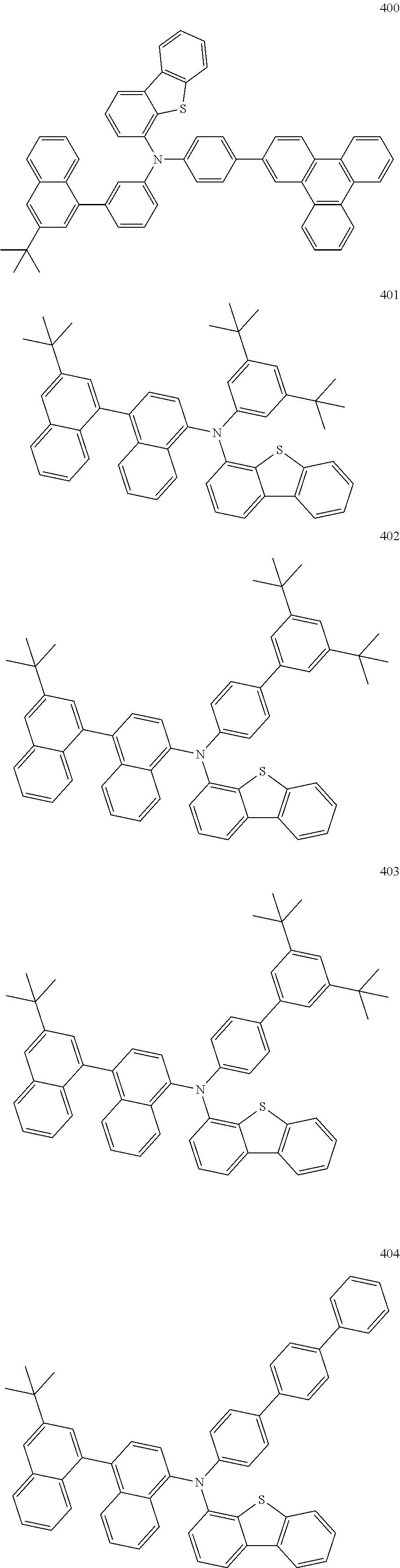

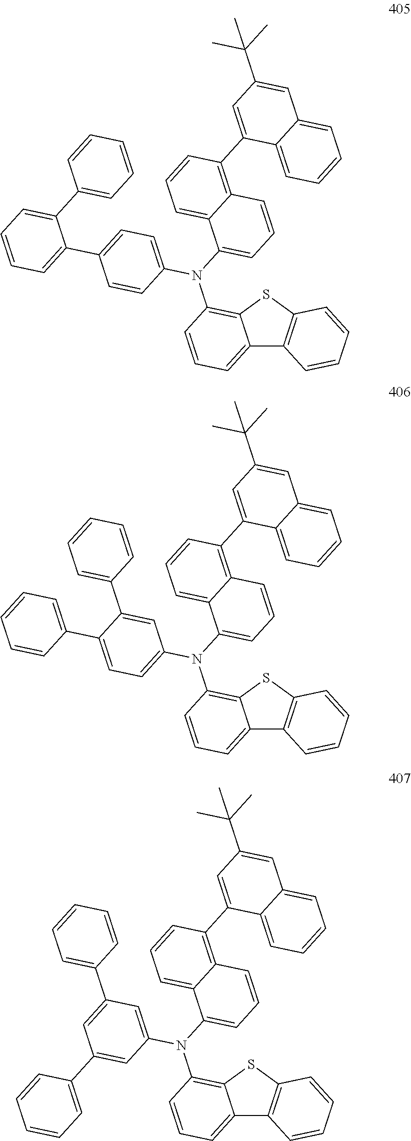

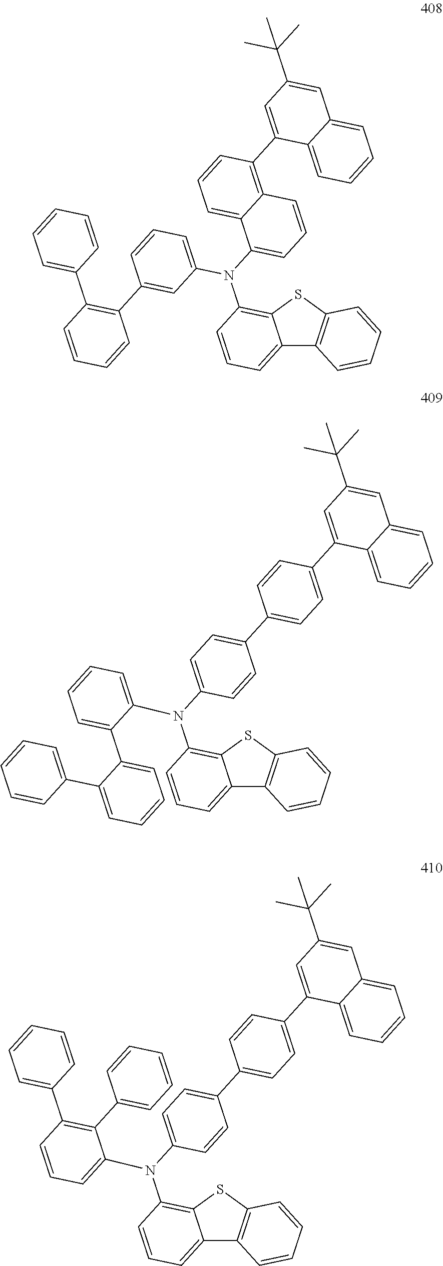 [SMILES: CC(C)(C)c1cc(-c2cccc3c(N(c4cccc(-c5ccccc5-c5ccccc5)c4)c4cccc5c4sc4ccccc45)cccc23)c2ccccc2c1 ; CC(C)(C)c1cc(-c2ccc(-c3ccc(N(c4ccccc4-c4ccccc4-c4ccccc4)c4cccc5c4sc4ccccc45)cc3)cc2)c2ccccc2c1 ; CC(C)(C)c1cc(-c2ccc(-c3ccc(N(c4cccc(-c5ccccc5)c4-c4ccccc4)c4cccc5c4sc4ccccc45)cc3)cc2)c2ccccc2c1]

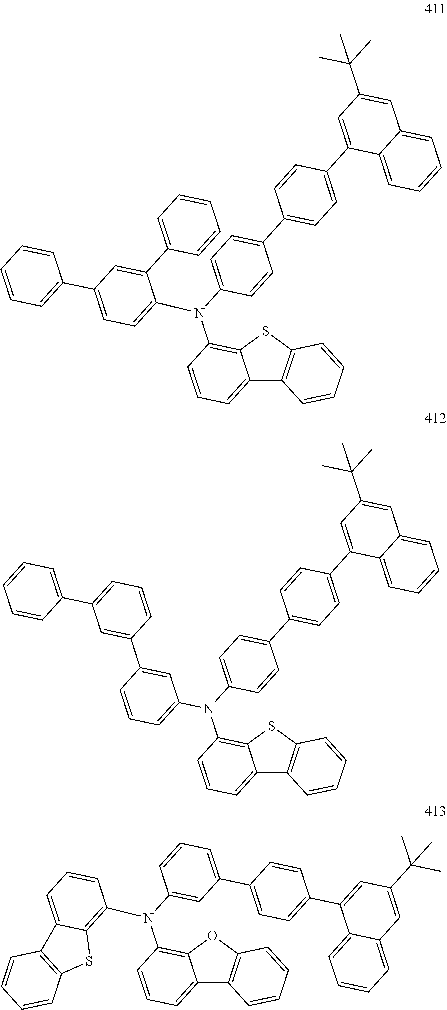 [SMILES: CC(C)(C)c1cc(-c2ccc(-c3ccc(N(c4ccc(-c5ccccc5)cc4-c4ccccc4)c4cccc5c4sc4ccccc45)cc3)cc2)c2ccccc2c1 ; CC(C)(C)c1cc(-c2ccc(-c3ccc(N(c4cccc(-c5cccc(-c6ccccc6)c5)c4)c4cccc5c4sc4ccccc45)cc3)cc2)c2ccccc2c1 ; CC(C)(C)c1cc(-c2ccc(-c3cccc(N(c4cccc5c4oc4ccccc45)c4cccc5c4sc4ccccc45)c3)cc2)c2ccccc2c1]

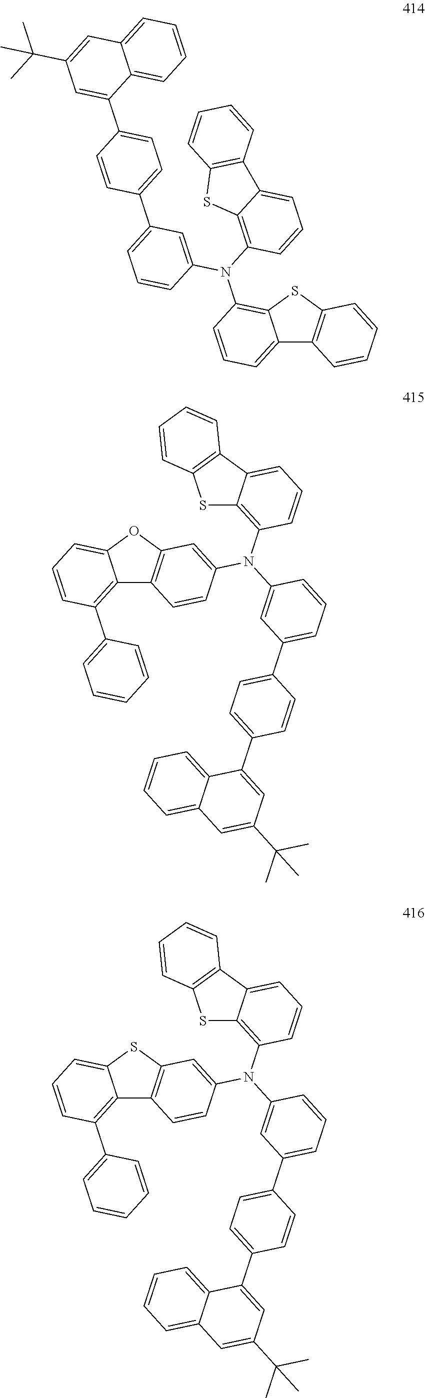 [SMILES: CC(C)(C)c1cc(-c2ccc(-c3cccc(N(c4cccc5c4sc4ccccc45)c4cccc5c4sc4ccccc45)c3)cc2)c2ccccc2c1 ; CC(C)(C)c1cc(-c2ccc(-c3cccc(N(c4ccc5c(c4)oc4cccc(-c6ccccc6)c45)c4cccc5c4sc4ccccc45)c3)cc2)c2ccccc2c1 ; CC(C)(C)c1cc(-c2ccc(-c3cccc(N(c4ccc5c(c4)sc4cccc(-c6ccccc6)c45)c4cccc5c4sc4ccccc45)c3)cc2)c2ccccc2c1]

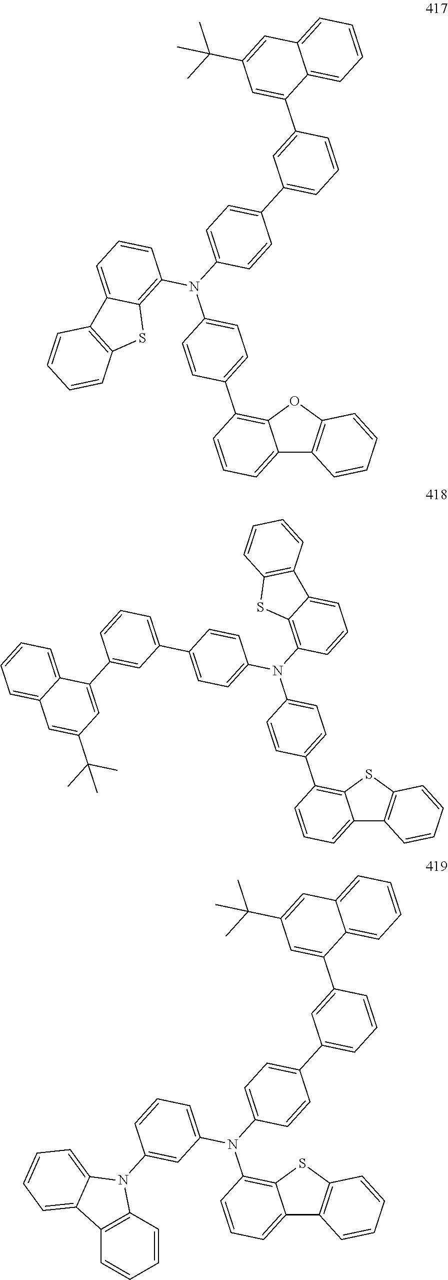

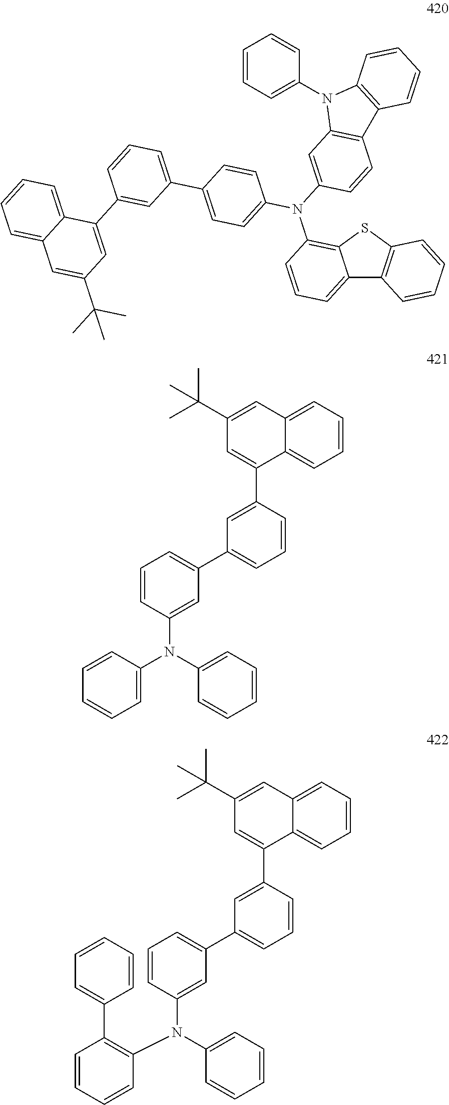

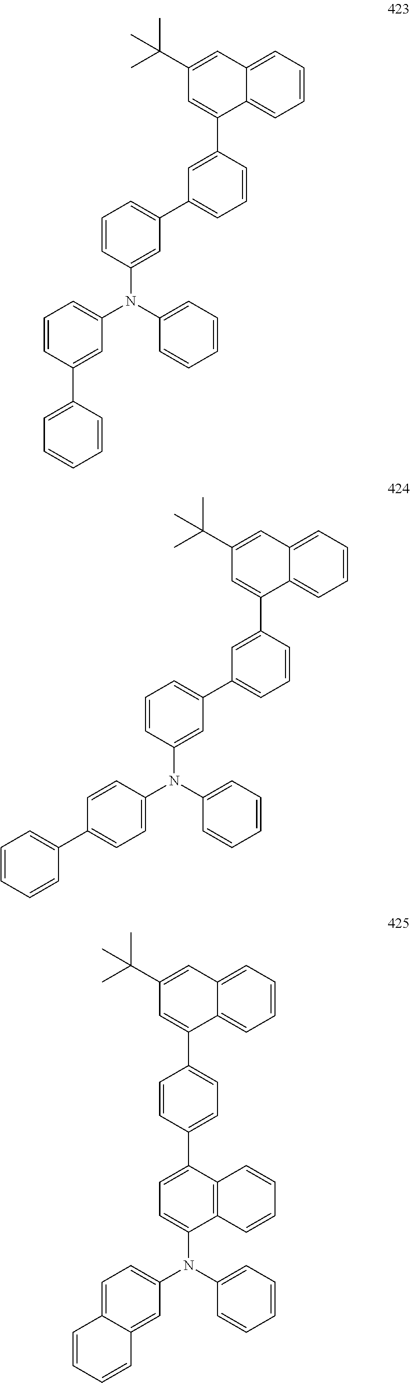 [SMILES: CC(C)(C)c1cc(-c2cccc(-c3cccc(N(c4ccccc4)c4cccc(-c5ccccc5)c4)c3)c2)c2ccccc2c1 ; CC(C)(C)c1cc(-c2cccc(-c3cccc(N(c4ccccc4)c4ccc(-c5ccccc5)cc4)c3)c2)c2ccccc2c1 ; CC(C)(C)c1cc(-c2ccc(-c3ccc(N(c4ccccc4)c4ccc5ccccc5c4)c4ccccc34)cc2)c2ccccc2c1]

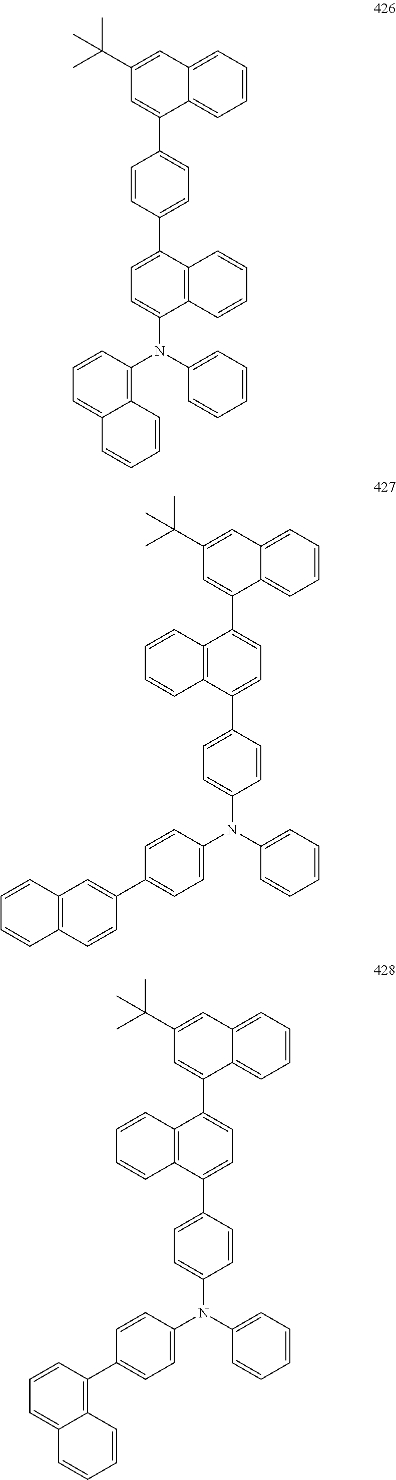 [SMILES: CC(C)(C)c1cc(-c2ccc(-c3ccc(N(c4ccccc4)c4cccc5ccccc45)c4ccccc34)cc2)c2ccccc2c1 ; CC(C)(C)c1cc(-c2ccc(-c3ccc(N(c4ccccc4)c4ccc(-c5ccc6ccccc6c5)cc4)cc3)c3ccccc23)c2ccccc2c1 ; CC(C)(C)c1cc(-c2ccc(-c3ccc(N(c4ccccc4)c4ccc(-c5cccc6ccccc56)cc4)cc3)c3ccccc23)c2ccccc2c1]

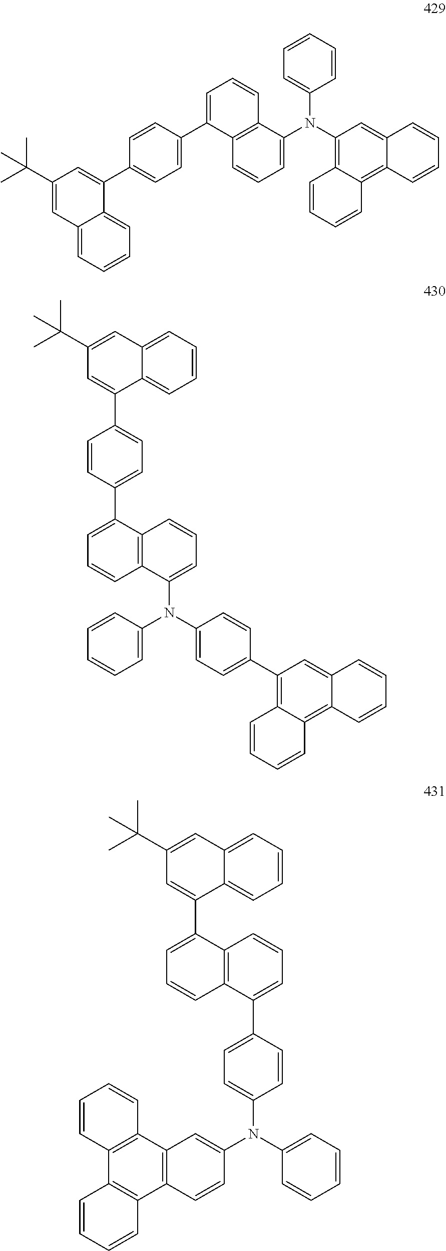 [SMILES: CC(C)(C)c1cc(-c2ccc(-c3cccc4c(N(c5ccccc5)c5cc6ccccc6c6ccccc56)cccc34)cc2)c2ccccc2c1 ; CC(C)(C)c1cc(-c2ccc(-c3cccc4c(N(c5ccccc5)c5ccc(-c6cc7ccccc7c7ccccc67)cc5)cccc34)cc2)c2ccccc2c1 ; CC(C)(C)c1cc(-c2cccc3c(-c4ccc(N(c5ccccc5)c5ccc6c7ccccc7c7ccccc7c6c5)cc4)cccc23)c2ccccc2c1]

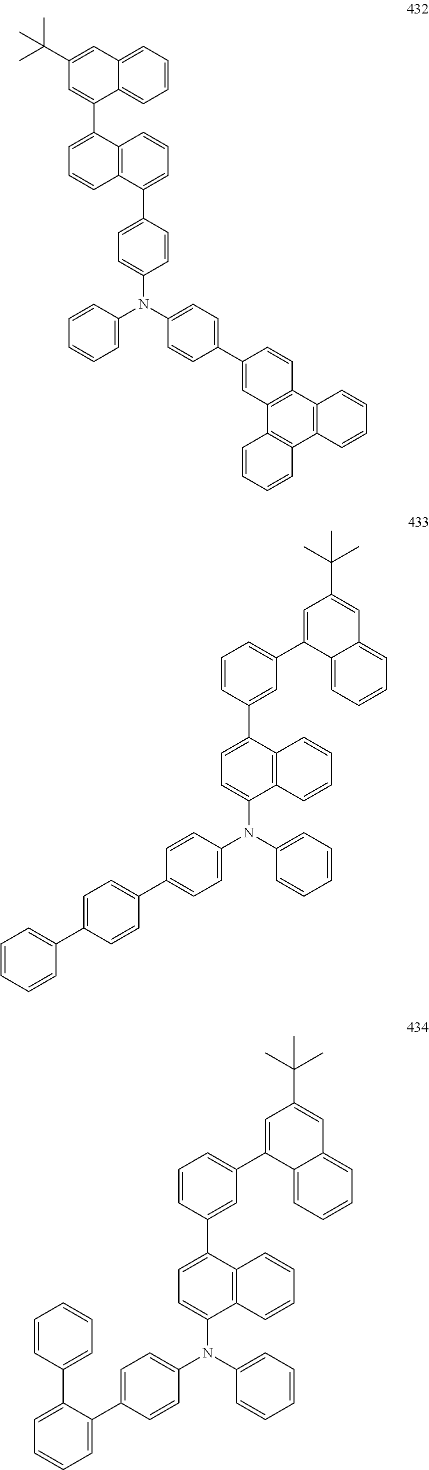 [SMILES: CC(C)(C)c1cc(-c2cccc3c(-c4ccc(N(c5ccccc5)c5ccc(-c6ccc7c8ccccc8c8ccccc8c7c6)cc5)cc4)cccc23)c2ccccc2c1 ; CC(C)(C)c1cc(-c2cccc(-c3ccc(N(c4ccccc4)c4ccc(-c5ccc(-c6ccccc6)cc5)cc4)c4ccccc34)c2)c2ccccc2c1 ; CC(C)(C)c1cc(-c2cccc(-c3ccc(N(c4ccccc4)c4ccc(-c5ccccc5-c5ccccc5)cc4)c4ccccc34)c2)c2ccccc2c1]

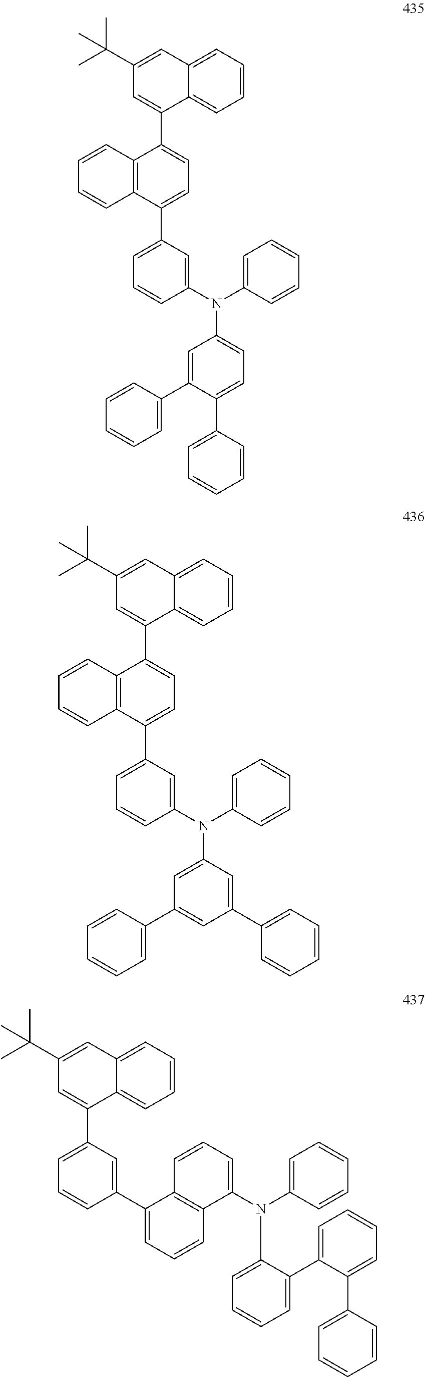 [SMILES: CC(C)(C)c1cc(-c2ccc(-c3cccc(N(c4ccccc4)c4ccc(-c5ccccc5)c(-c5ccccc5)c4)c3)c3ccccc23)c2ccccc2c1 ; CC(C)(C)c1cc(-c2ccc(-c3cccc(N(c4ccccc4)c4cc(-c5ccccc5)cc(-c5ccccc5)c4)c3)c3ccccc23)c2ccccc2c1 ; CC(C)(C)c1cc(-c2cccc(-c3cccc4c(N(c5ccccc5)c5ccccc5-c5ccccc5-c5ccccc5)cccc34)c2)c2ccccc2c1]

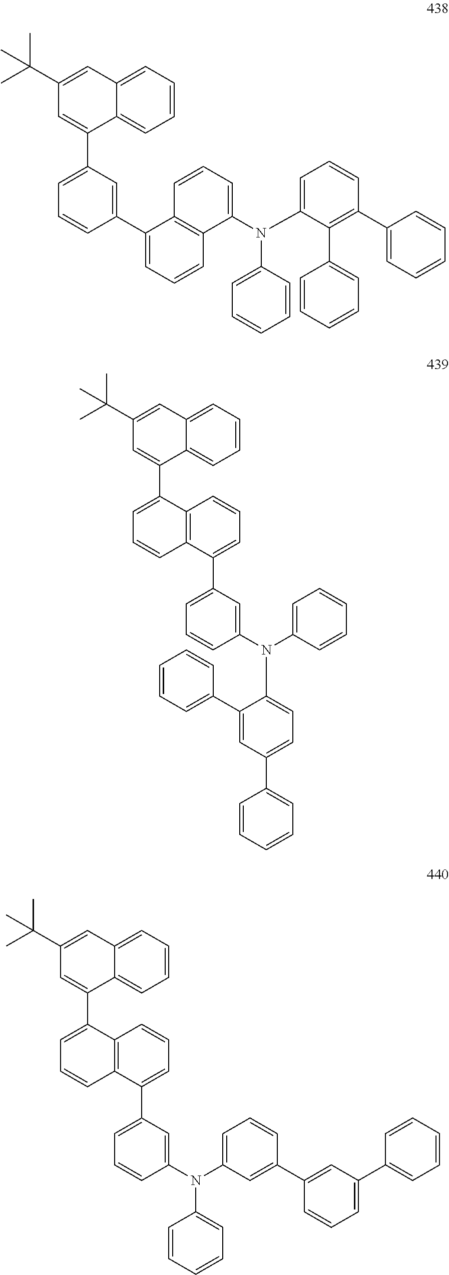 [SMILES: CC(C)(C)c1cc(-c2cccc(-c3cccc4c(N(c5ccccc5)c5cccc(-c6ccccc6)c5-c5ccccc5)cccc34)c2)c2ccccc2c1 ; CC(C)(C)c1cc(-c2cccc3c(-c4cccc(N(c5ccccc5)c5ccc(-c6ccccc6)cc5-c5ccccc5)c4)cccc23)c2ccccc2c1 ; CC(C)(C)c1cc(-c2cccc3c(-c4cccc(N(c5ccccc5)c5cccc(-c6cccc(-c7ccccc7)c6)c5)c4)cccc23)c2ccccc2c1]

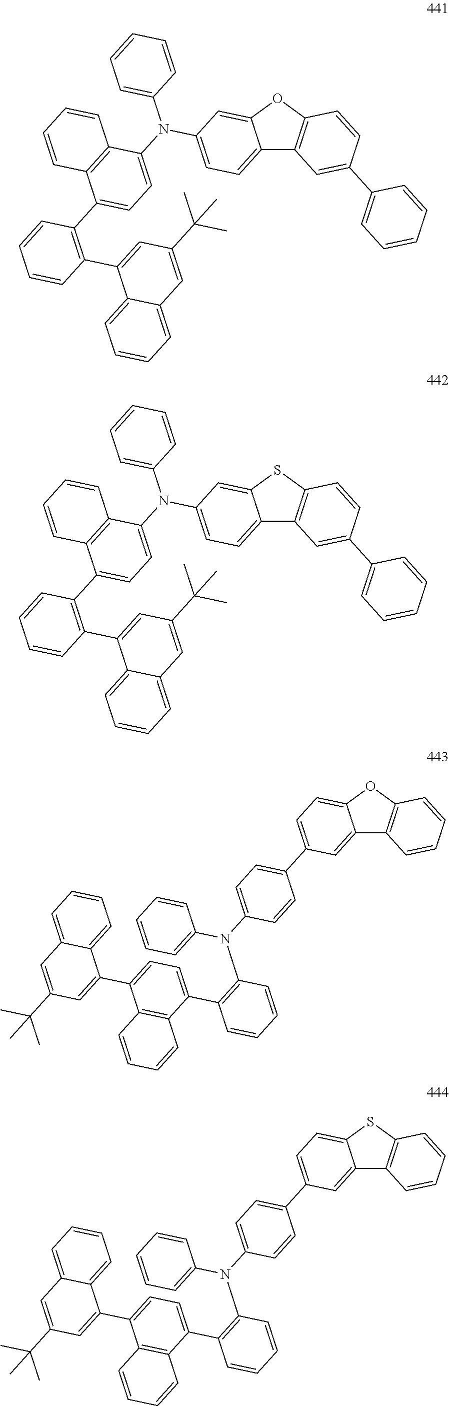

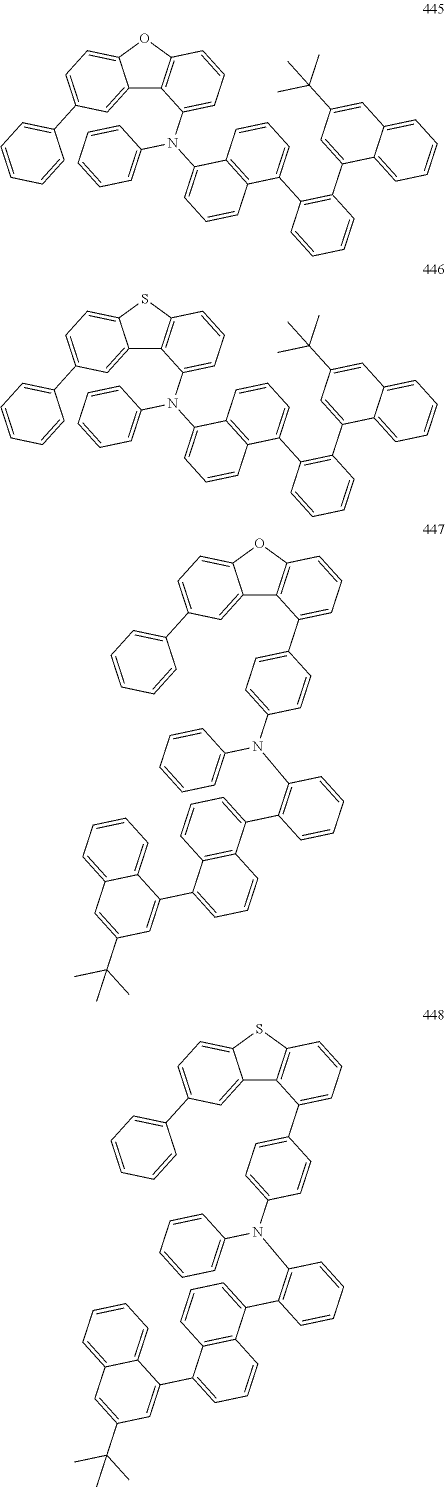

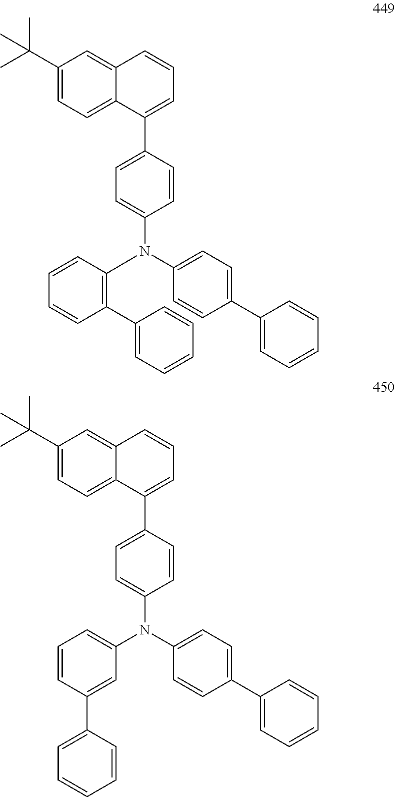 [SMILES: CC(C)(C)c1ccc2c(-c3ccc(N(c4ccc(-c5ccccc5)cc4)c4ccccc4-c4ccccc4)cc3)cccc2c1 ; CC(C)(C)c1ccc2c(-c3ccc(N(c4ccc(-c5ccccc5)cc4)c4cccc(-c5ccccc5)c4)cc3)cccc2c1]

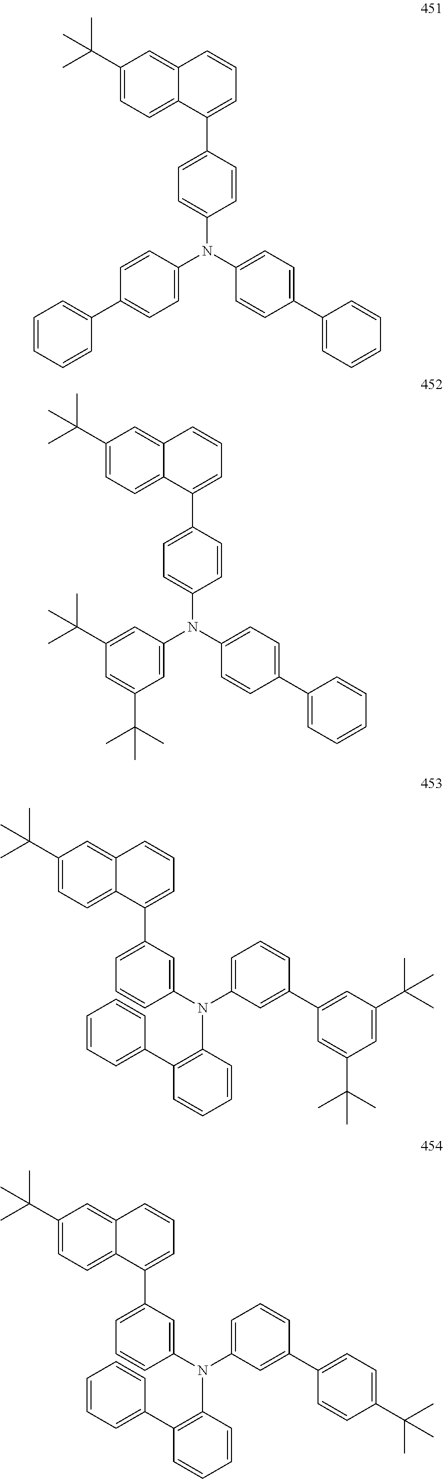

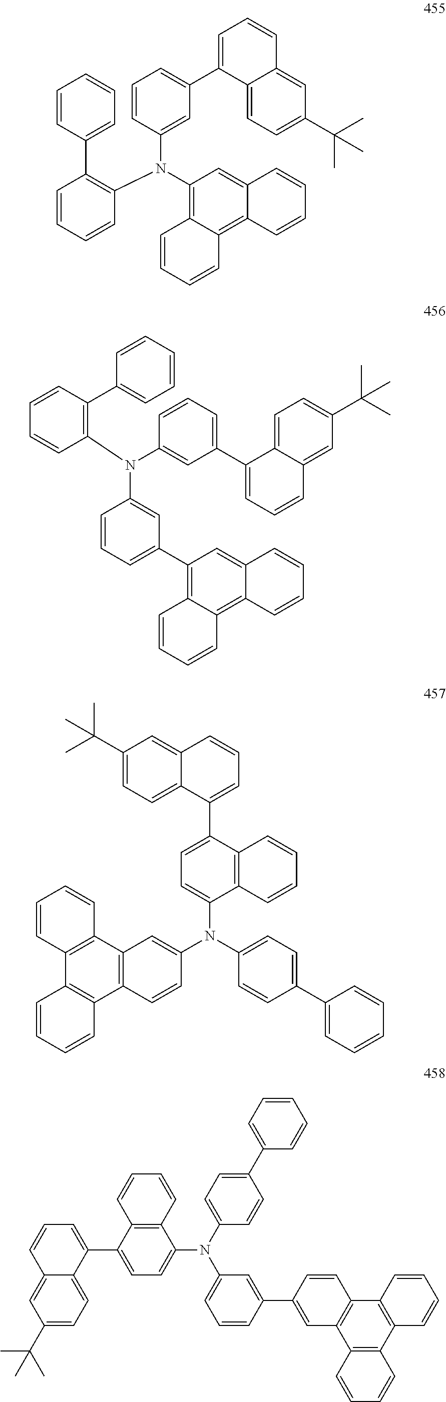

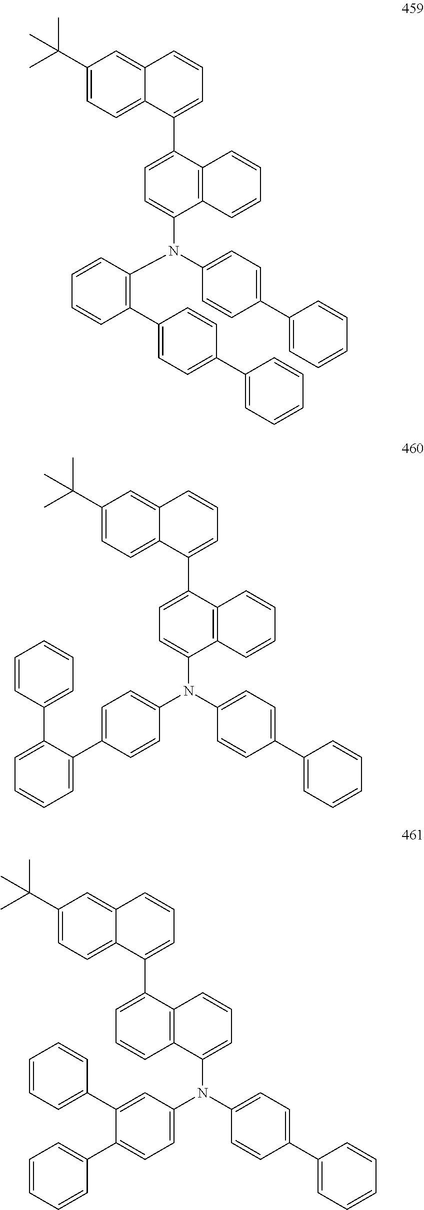 [SMILES: CC(C)(C)c1ccc2c(-c3ccc(N(c4ccc(-c5ccccc5)cc4)c4ccccc4-c4ccc(-c5ccccc5)cc4)c4ccccc34)cccc2c1 ; CC(C)(C)c1ccc2c(-c3ccc(N(c4ccc(-c5ccccc5)cc4)c4ccc(-c5ccccc5-c5ccccc5)cc4)c4ccccc34)cccc2c1 ; CC(C)(C)c1ccc2c(-c3cccc4c(N(c5ccc(-c6ccccc6)cc5)c5ccc(-c6ccccc6)c(-c6ccccc6)c5)cccc34)cccc2c1]

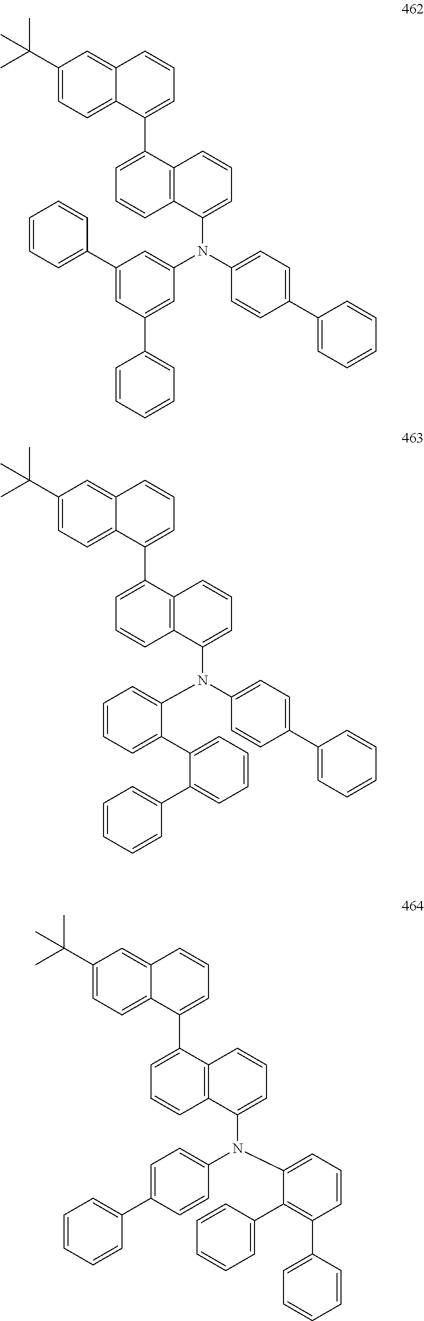 [SMILES: CC(C)(C)c1ccc2c(-c3cccc4c(N(c5ccc(-c6ccccc6)cc5)c5cc(-c6ccccc6)cc(-c6ccccc6)c5)cccc34)cccc2c1 ; CC(C)(C)c1ccc2c(-c3cccc4c(N(c5ccc(-c6ccccc6)cc5)c5ccccc5-c5ccccc5-c5ccccc5)cccc34)cccc2c1 ; CC(C)(C)c1ccc2c(-c3cccc4c(N(c5ccc(-c6ccccc6)cc5)c5cccc(-c6ccccc6)c5-c5ccccc5)cccc34)cccc2c1]

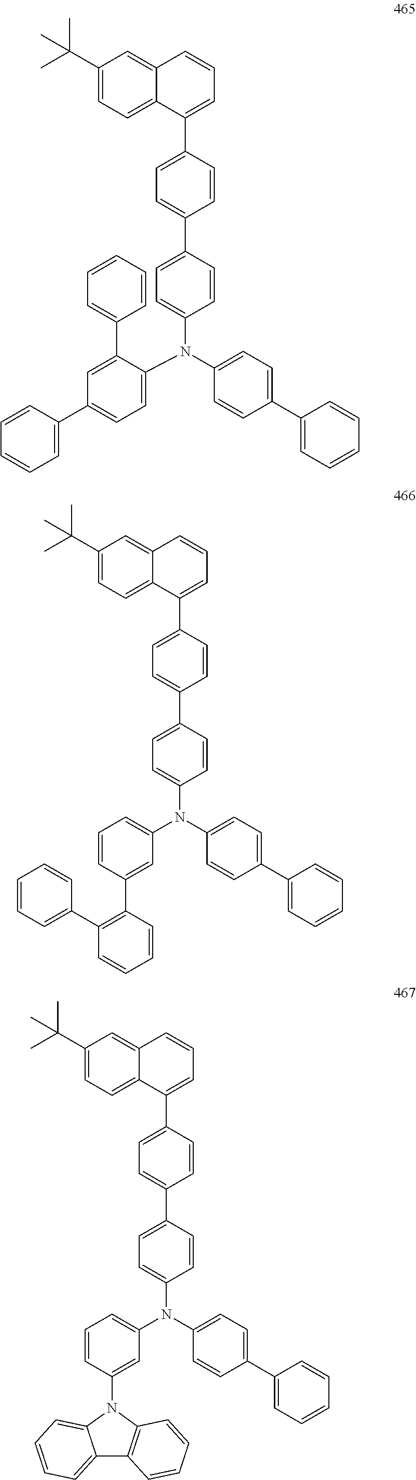 [SMILES: CC(C)(C)c1ccc2c(-c3ccc(-c4ccc(N(c5ccc(-c6ccccc6)cc5)c5ccc(-c6ccccc6)cc5-c5ccccc5)cc4)cc3)cccc2c1 ; CC(C)(C)c1ccc2c(-c3ccc(-c4ccc(N(c5ccc(-c6ccccc6)cc5)c5cccc(-c6ccccc6-c6ccccc6)c5)cc4)cc3)cccc2c1 ; CC(C)(C)c1ccc2c(-c3ccc(-c4ccc(N(c5ccc(-c6ccccc6)cc5)c5cccc(-n6c7ccccc7c7ccccc76)c5)cc4)cc3)cccc2c1]

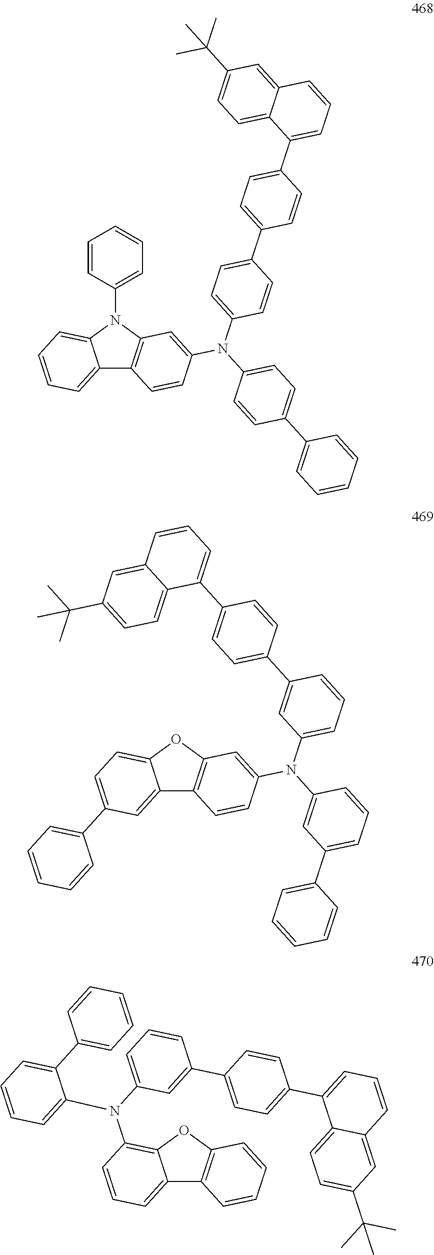 [SMILES: CC(C)(C)c1ccc2c(-c3ccc(-c4ccc(N(c5ccc(-c6ccccc6)cc5)c5ccc6c7ccccc7n(-c7ccccc7)c6c5)cc4)cc3)cccc2c1 ; CC(C)(C)c1ccc2c(-c3ccc(-c4cccc(N(c5cccc(-c6ccccc6)c5)c5ccc6c(c5)oc5ccc(-c7ccccc7)cc56)c4)cc3)cccc2c1 ; CC(C)(C)c1ccc2c(-c3ccc(-c4cccc(N(c5ccccc5-c5ccccc5)c5cccc6c5oc5ccccc56)c4)cc3)cccc2c1]

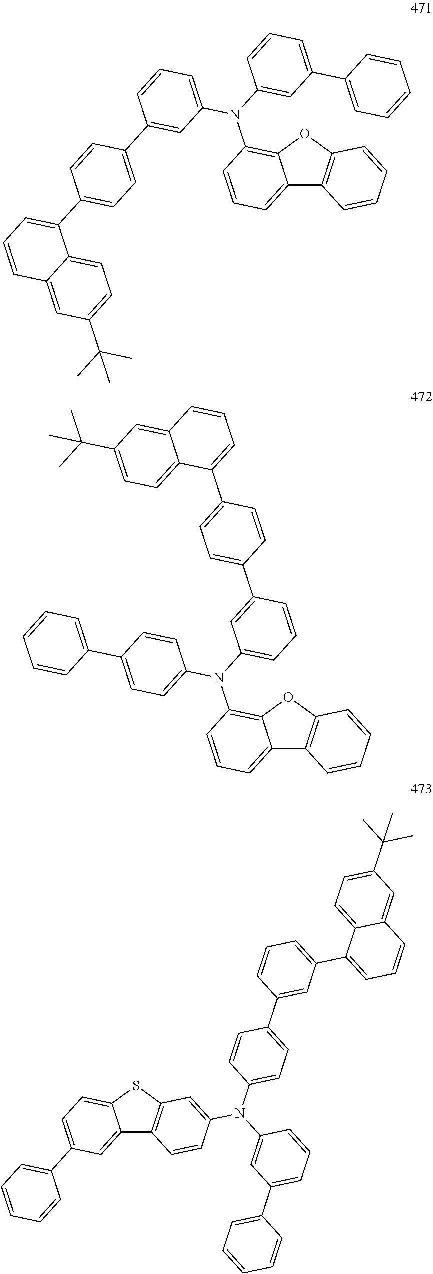

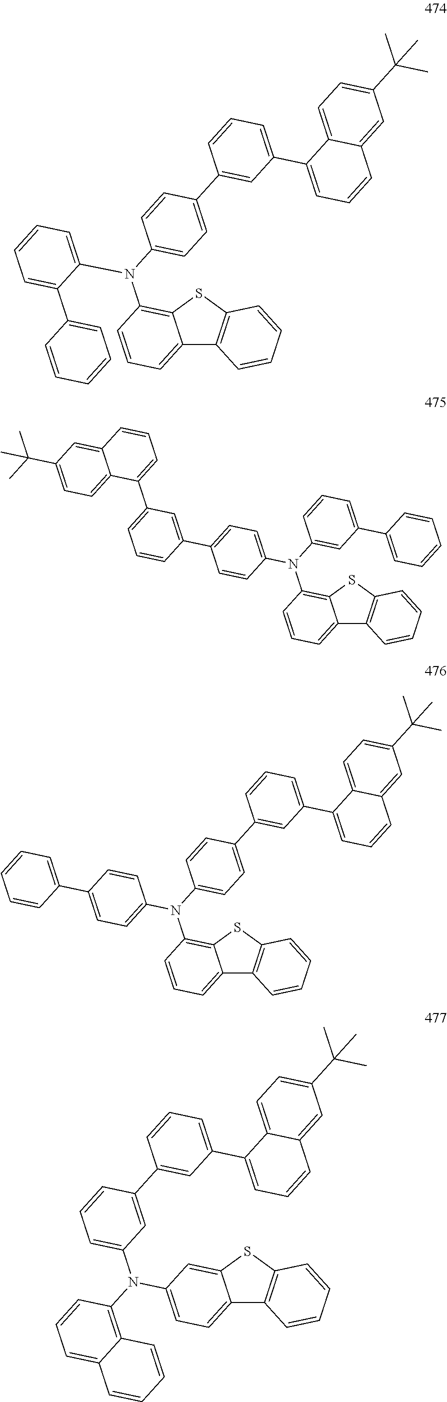

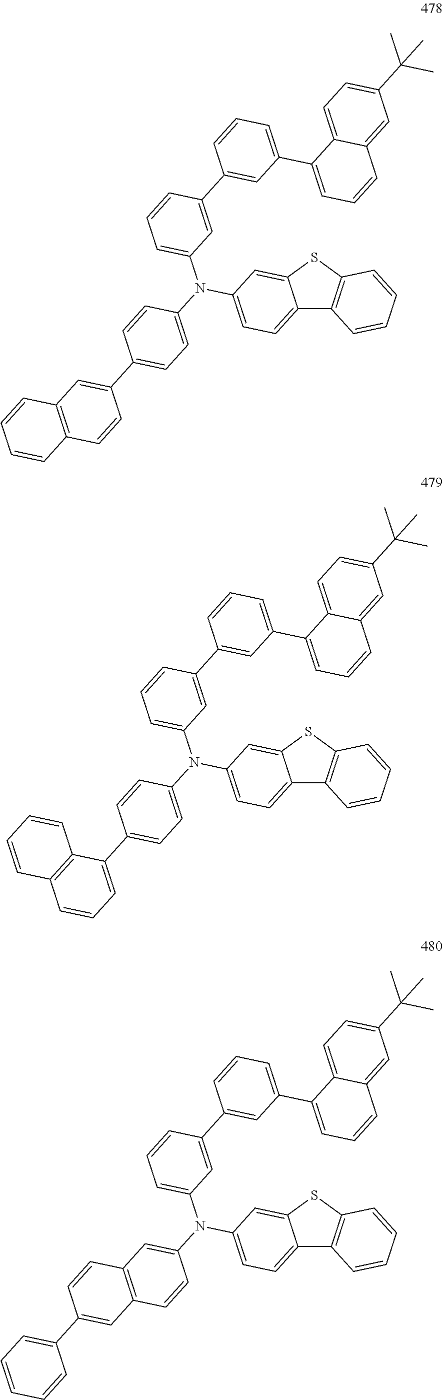 [SMILES: CC(C)(C)c1ccc2c(-c3cccc(-c4cccc(N(c5ccc(-c6ccc7ccccc7c6)cc5)c5ccc6c(c5)sc5ccccc56)c4)c3)cccc2c1 ; CC(C)(C)c1ccc2c(-c3cccc(-c4cccc(N(c5ccc(-c6cccc7ccccc67)cc5)c5ccc6c(c5)sc5ccccc56)c4)c3)cccc2c1 ; CC(C)(C)c1ccc2c(-c3cccc(-c4cccc(N(c5ccc6cc(-c7ccccc7)ccc6c5)c5ccc6c(c5)sc5ccccc56)c4)c3)cccc2c1]

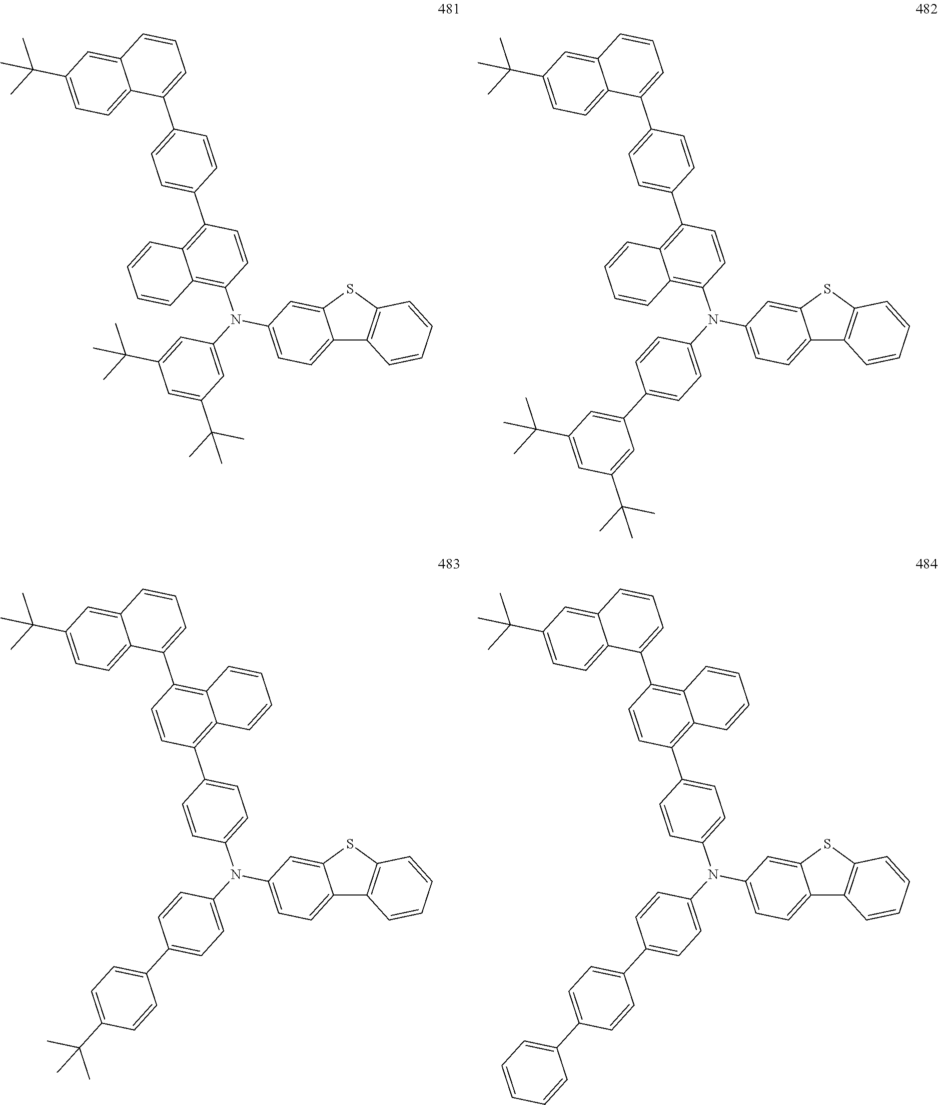 [SMILES: CC(C)(C)c1cc(N(c2ccc3c(c2)sc2ccccc23)c2ccc(-c3ccc(-c4cccc5cc(C(C)(C)C)ccc45)cc3)c3ccccc23)cc(C(C)(C)C)c1 ; CC(C)(C)c1cc(-c2ccc(N(c3ccc4c(c3)sc3ccccc34)c3ccc(-c4ccc(-c5cccc6cc(C(C)(C)C)ccc56)cc4)c4ccccc34)cc2)cc(C(C)(C)C)c1 ; CC(C)(C)c1ccc(-c2ccc(N(c3ccc(-c4ccc(-c5cccc6cc(C(C)(C)C)ccc56)c5ccccc45)cc3)c3ccc4c(c3)sc3ccccc34)cc2)cc1 ; CC(C)(C)c1ccc2c(-c3ccc(-c4ccc(N(c5ccc(-c6ccc(-c7ccccc7)cc6)cc5)c5ccc6c(c5)sc5ccccc56)cc4)c4ccccc34)cccc2c1]

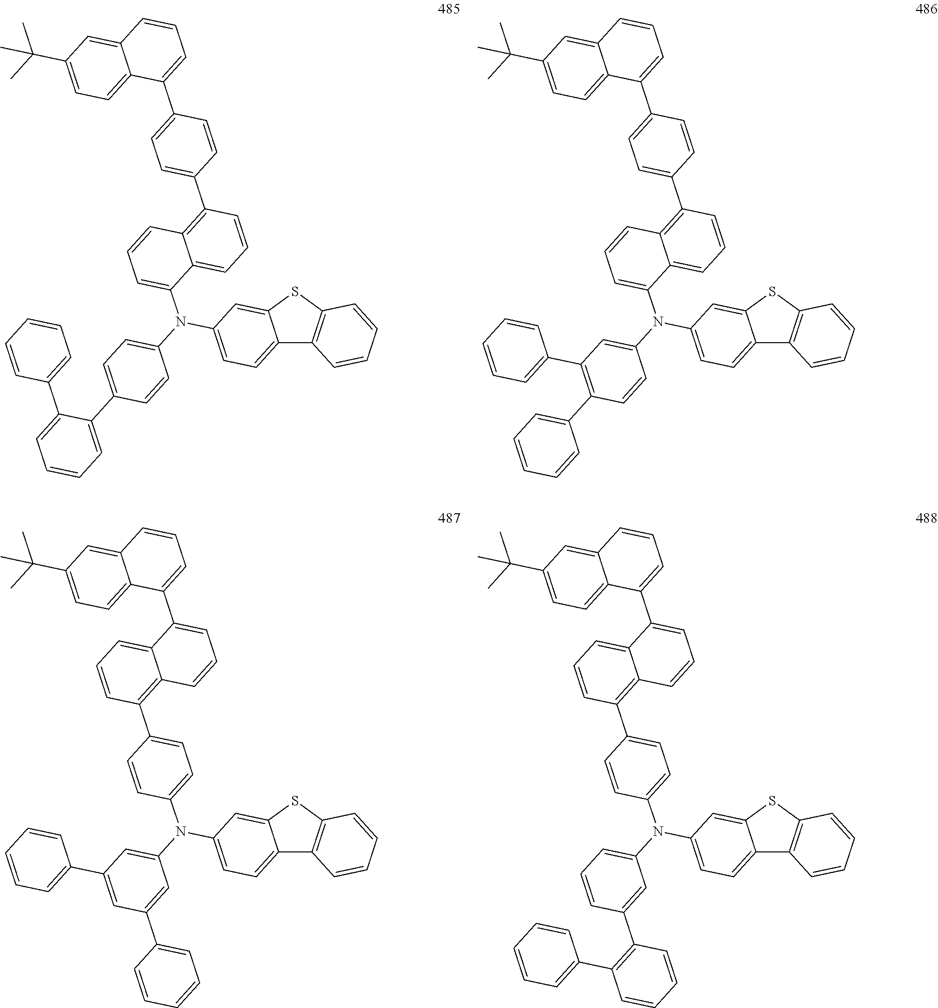 [SMILES: CC(C)(C)c1ccc2c(-c3ccc(-c4cccc5c(N(c6ccc(-c7ccccc7-c7ccccc7)cc6)c6ccc7c(c6)sc6ccccc67)cccc45)cc3)cccc2c1 ; CC(C)(C)c1ccc2c(-c3ccc(-c4cccc5c(N(c6ccc(-c7ccccc7)c(-c7ccccc7)c6)c6ccc7c(c6)sc6ccccc67)cccc45)cc3)cccc2c1 ; CC(C)(C)c1ccc2c(-c3cccc4c(-c5ccc(N(c6cc(-c7ccccc7)cc(-c7ccccc7)c6)c6ccc7c(c6)sc6ccccc67)cc5)cccc34)cccc2c1 ; CC(C)(C)c1ccc2c(-c3cccc4c(-c5ccc(N(c6cccc(-c7ccccc7-c7ccccc7)c6)c6ccc7c(c6)sc6ccccc67)cc5)cccc34)cccc2c1]

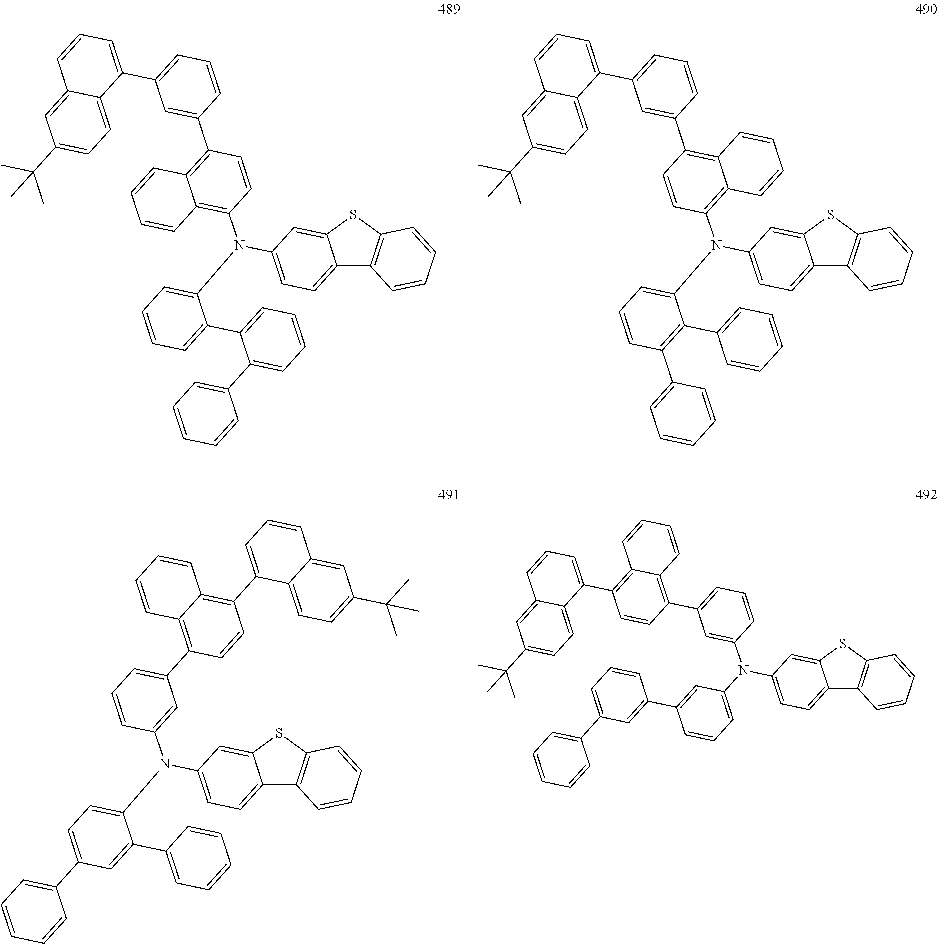

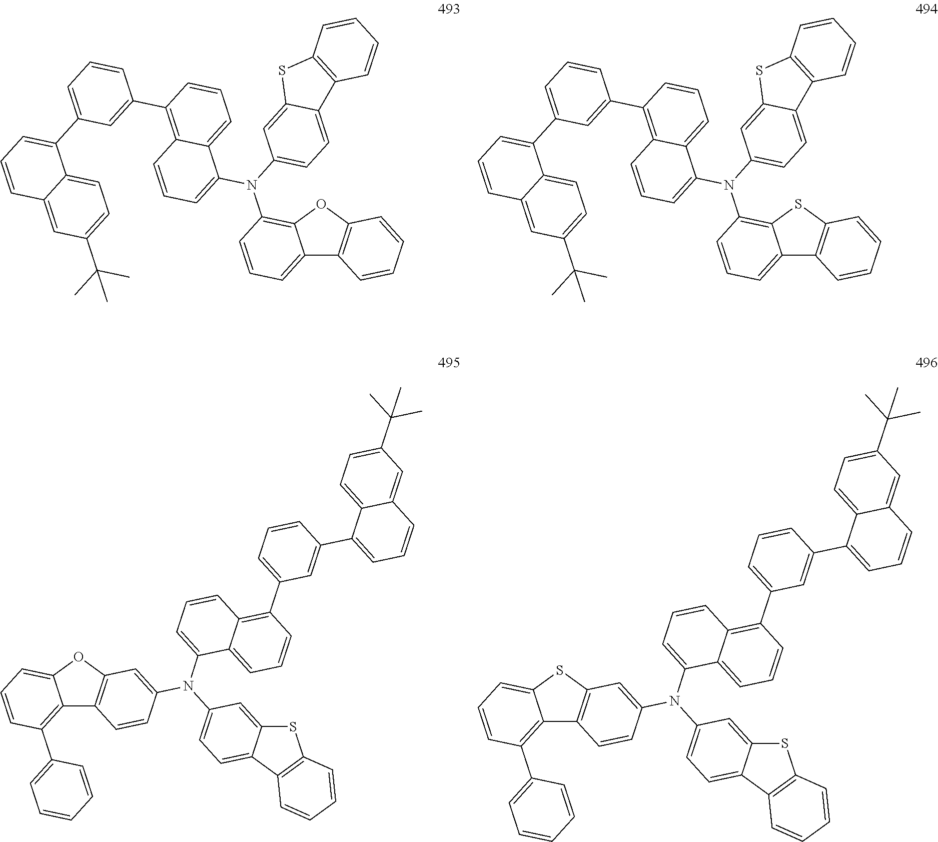 [SMILES: CC(C)(C)c1ccc2c(-c3cccc(-c4cccc5c(N(c6ccc7c(c6)sc6ccccc67)c6cccc7c6oc6ccccc67)cccc45)c3)cccc2c1 ; CC(C)(C)c1ccc2c(-c3cccc(-c4cccc5c(N(c6ccc7c(c6)sc6ccccc67)c6cccc7c6sc6ccccc67)cccc45)c3)cccc2c1 ; CC(C)(C)c1ccc2c(-c3cccc(-c4cccc5c(N(c6ccc7c(c6)oc6cccc(-c8ccccc8)c67)c6ccc7c(c6)sc6ccccc67)cccc45)c3)cccc2c1 ; CC(C)(C)c1ccc2c(-c3cccc(-c4cccc5c(N(c6ccc7c(c6)sc6ccccc67)c6ccc7c(c6)sc6cccc(-c8ccccc8)c67)cccc45)c3)cccc2c1]

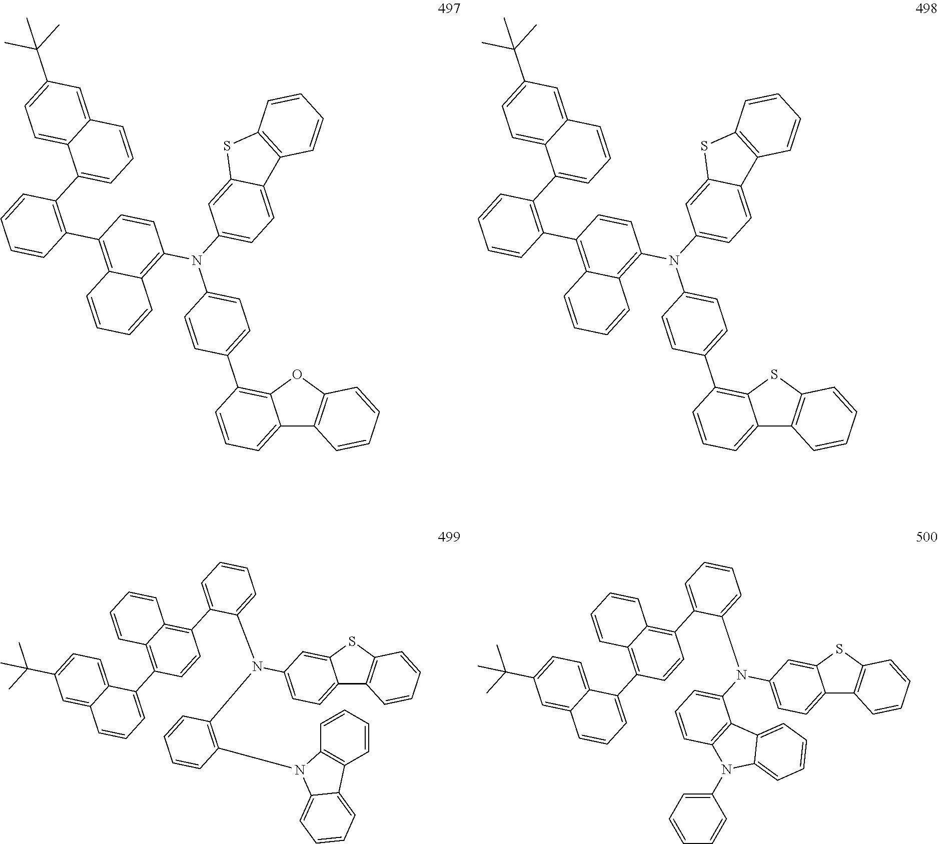 [SMILES: CC(C)(C)c1ccc2c(-c3ccccc3-c3ccc(N(c4ccc(-c5cccc6c5oc5ccccc56)cc4)c4ccc5c(c4)sc4ccccc45)c4ccccc34)cccc2c1 ; CC(C)(C)c1ccc2c(-c3ccccc3-c3ccc(N(c4ccc(-c5cccc6c5sc5ccccc56)cc4)c4ccc5c(c4)sc4ccccc45)c4ccccc34)cccc2c1 ; CC(C)(C)c1ccc2c(-c3ccc(-c4ccccc4N(c4ccc5c(c4)sc4ccccc45)c4ccccc4-n4c5ccccc5c5ccccc54)c4ccccc34)cccc2c1 ; CC(C)(C)c1ccc2c(-c3ccc(-c4ccccc4N(c4ccc5c(c4)sc4ccccc45)c4cccc5c4c4ccccc4n5-c4ccccc4)c4ccccc34)cccc2c1]

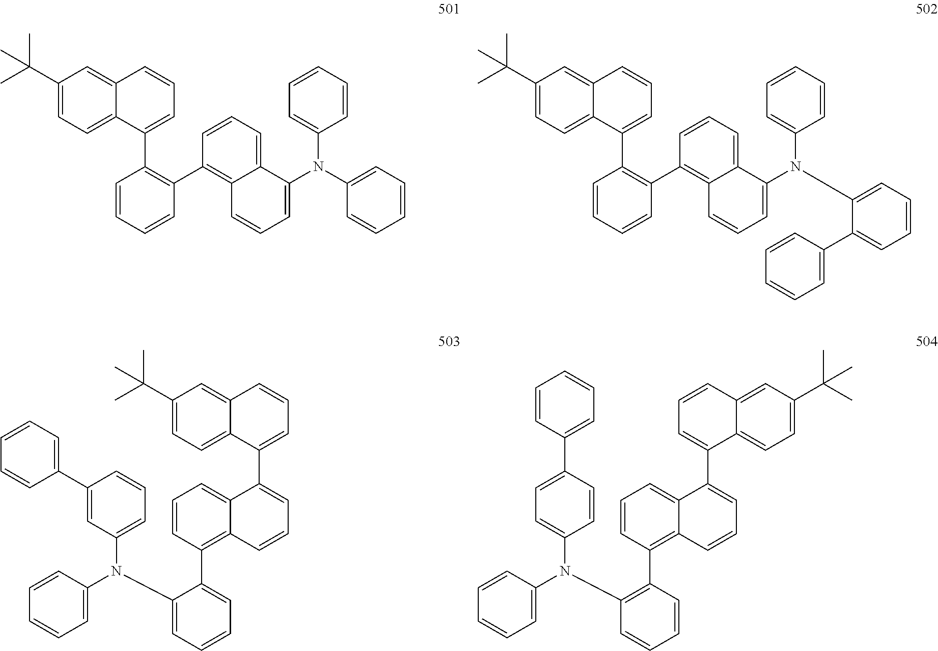 [SMILES: CC(C)(C)c1ccc2c(-c3ccccc3-c3cccc4c(N(c5ccccc5)c5ccccc5)cccc34)cccc2c1 ; CC(C)(C)c1ccc2c(-c3ccccc3-c3cccc4c(N(c5ccccc5)c5ccccc5-c5ccccc5)cccc34)cccc2c1 ; CC(C)(C)c1ccc2c(-c3cccc4c(-c5ccccc5N(c5ccccc5)c5cccc(-c6ccccc6)c5)cccc34)cccc2c1 ; CC(C)(C)c1ccc2c(-c3cccc4c(-c5ccccc5N(c5ccccc5)c5ccc(-c6ccccc6)cc5)cccc34)cccc2c1]

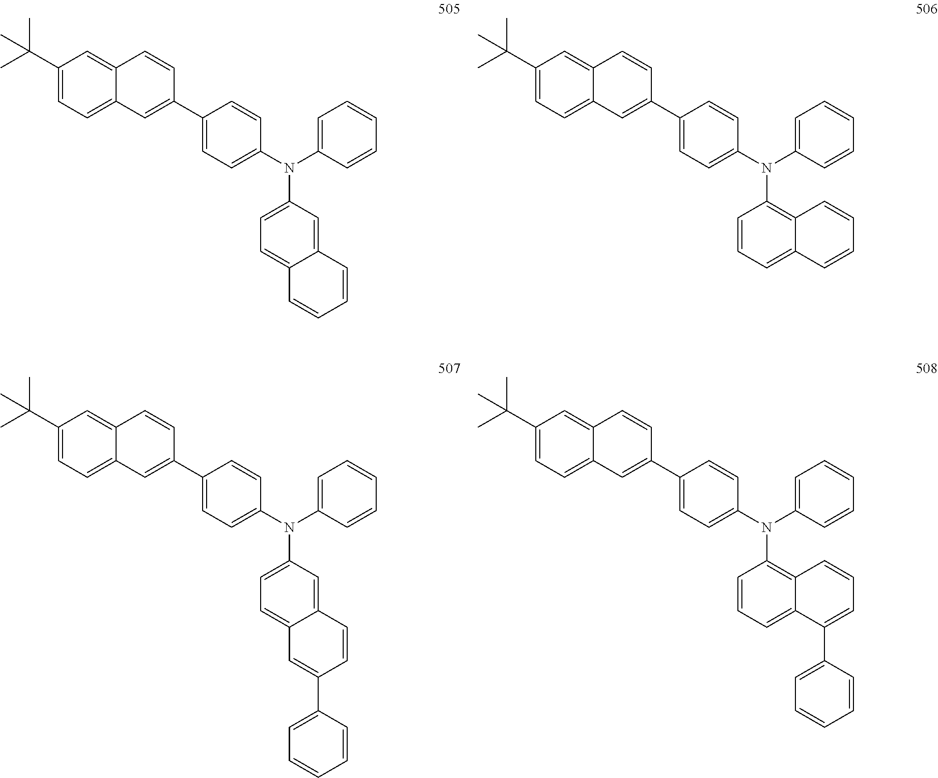 [SMILES: CC(C)(C)c1ccc2cc(-c3ccc(N(c4ccccc4)c4ccc5ccccc5c4)cc3)ccc2c1 ; CC(C)(C)c1ccc2cc(-c3ccc(N(c4ccccc4)c4cccc5ccccc45)cc3)ccc2c1 ; CC(C)(C)c1ccc2cc(-c3ccc(N(c4ccccc4)c4ccc5cc(-c6ccccc6)ccc5c4)cc3)ccc2c1 ; CC(C)(C)c1ccc2cc(-c3ccc(N(c4ccccc4)c4cccc5c(-c6ccccc6)cccc45)cc3)ccc2c1]

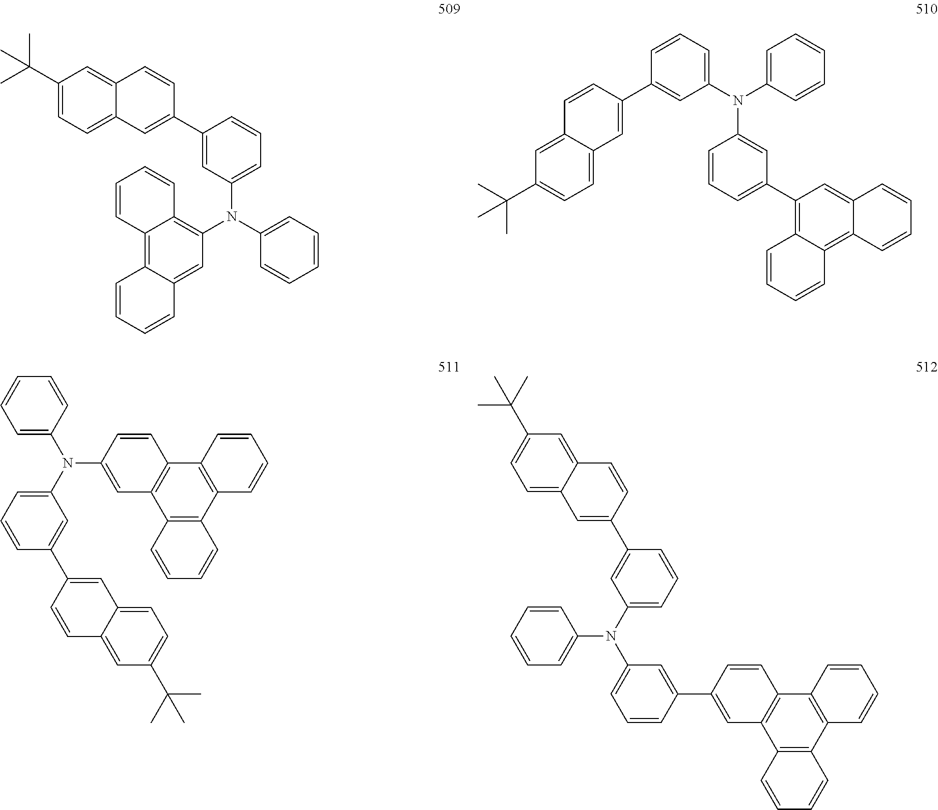 [SMILES: CC(C)(C)c1ccc2cc(-c3cccc(N(c4ccccc4)c4cc5ccccc5c5ccccc45)c3)ccc2c1 ; CC(C)(C)c1ccc2cc(-c3cccc(N(c4ccccc4)c4cccc(-c5cc6ccccc6c6ccccc56)c4)c3)ccc2c1 ; CC(C)(C)c1ccc2cc(-c3cccc(N(c4ccccc4)c4ccc5c6ccccc6c6ccccc6c5c4)c3)ccc2c1 ; CC(C)(C)c1ccc2cc(-c3cccc(N(c4ccccc4)c4cccc(-c5ccc6c7ccccc7c7ccccc7c6c5)c4)c3)ccc2c1]

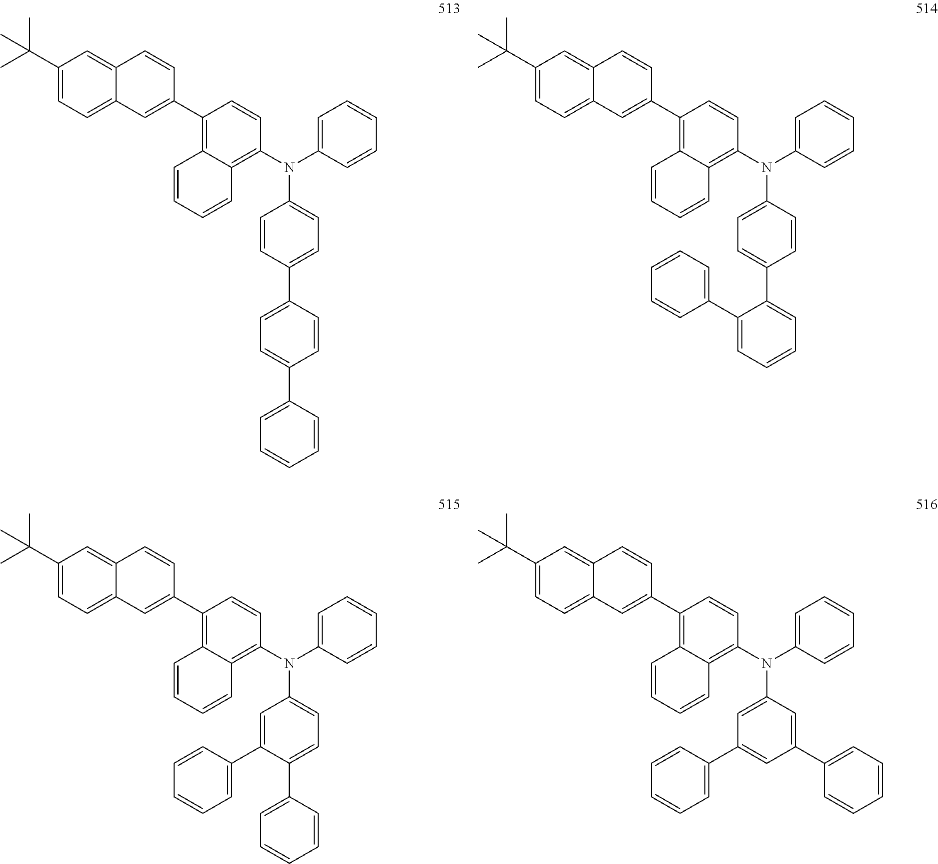 [SMILES: CC(C)(C)c1ccc2cc(-c3ccc(N(c4ccccc4)c4ccc(-c5ccc(-c6ccccc6)cc5)cc4)c4ccccc34)ccc2c1 ; CC(C)(C)c1ccc2cc(-c3ccc(N(c4ccccc4)c4ccc(-c5ccccc5-c5ccccc5)cc4)c4ccccc34)ccc2c1 ; CC(C)(C)c1ccc2cc(-c3ccc(N(c4ccccc4)c4ccc(-c5ccccc5)c(-c5ccccc5)c4)c4ccccc34)ccc2c1 ; CC(C)(C)c1ccc2cc(-c3ccc(N(c4ccccc4)c4cc(-c5ccccc5)cc(-c5ccccc5)c4)c4ccccc34)ccc2c1]

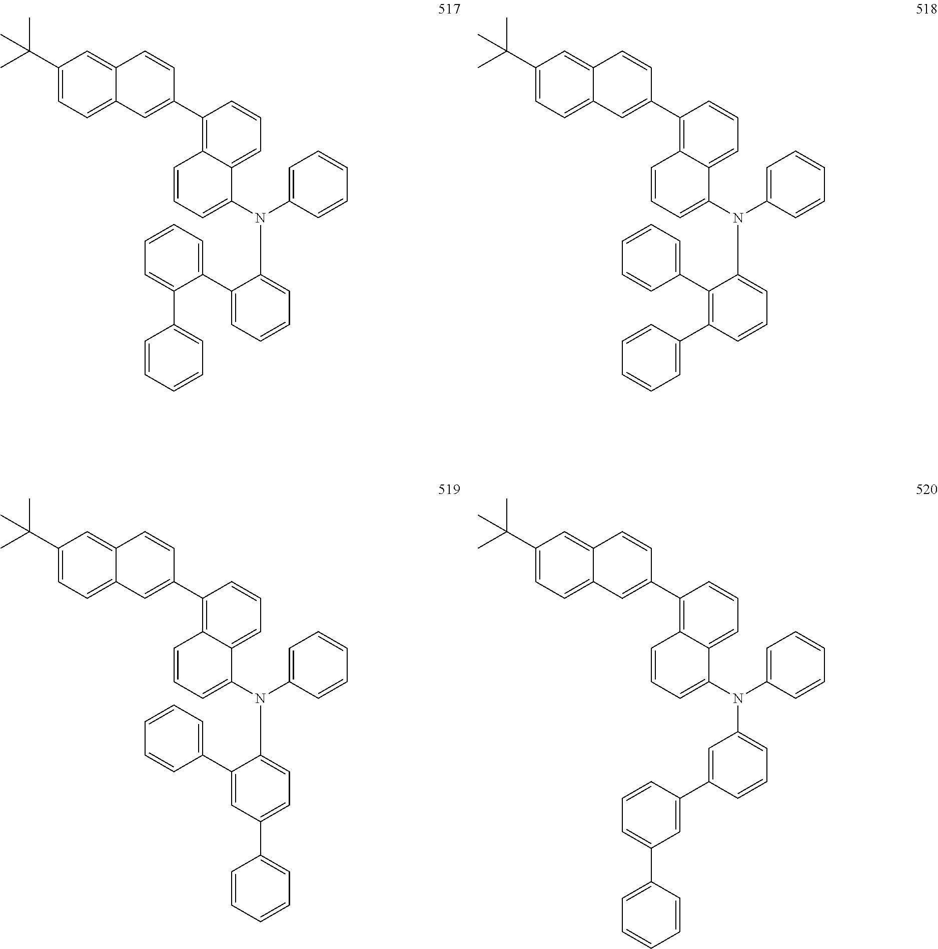 [SMILES: CC(C)(C)c1ccc2cc(-c3cccc4c(N(c5ccccc5)c5ccccc5-c5ccccc5-c5ccccc5)cccc34)ccc2c1 ; CC(C)(C)c1ccc2cc(-c3cccc4c(N(c5ccccc5)c5cccc(-c6ccccc6)c5-c5ccccc5)cccc34)ccc2c1 ; CC(C)(C)c1ccc2cc(-c3cccc4c(N(c5ccccc5)c5ccc(-c6ccccc6)cc5-c5ccccc5)cccc34)ccc2c1 ; CC(C)(C)c1ccc2cc(-c3cccc4c(N(c5ccccc5)c5cccc(-c6cccc(-c7ccccc7)c6)c5)cccc34)ccc2c1]

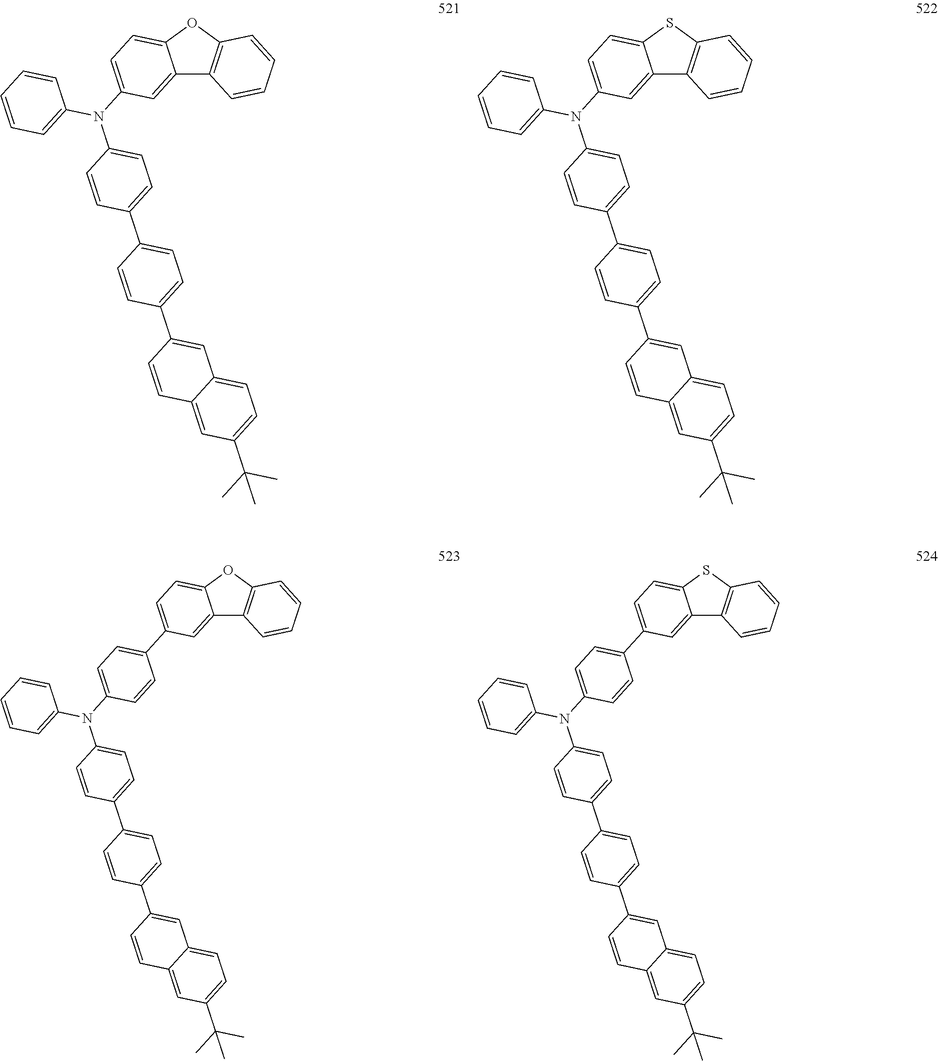 [SMILES: CC(C)(C)c1ccc2cc(-c3ccc(-c4ccc(N(c5ccccc5)c5ccc6oc7ccccc7c6c5)cc4)cc3)ccc2c1 ; CC(C)(C)c1ccc2cc(-c3ccc(-c4ccc(N(c5ccccc5)c5ccc6sc7ccccc7c6c5)cc4)cc3)ccc2c1 ; CC(C)(C)c1ccc2cc(-c3ccc(-c4ccc(N(c5ccccc5)c5ccc(-c6ccc7oc8ccccc8c7c6)cc5)cc4)cc3)ccc2c1 ; CC(C)(C)c1ccc2cc(-c3ccc(-c4ccc(N(c5ccccc5)c5ccc(-c6ccc7sc8ccccc8c7c6)cc5)cc4)cc3)ccc2c1]

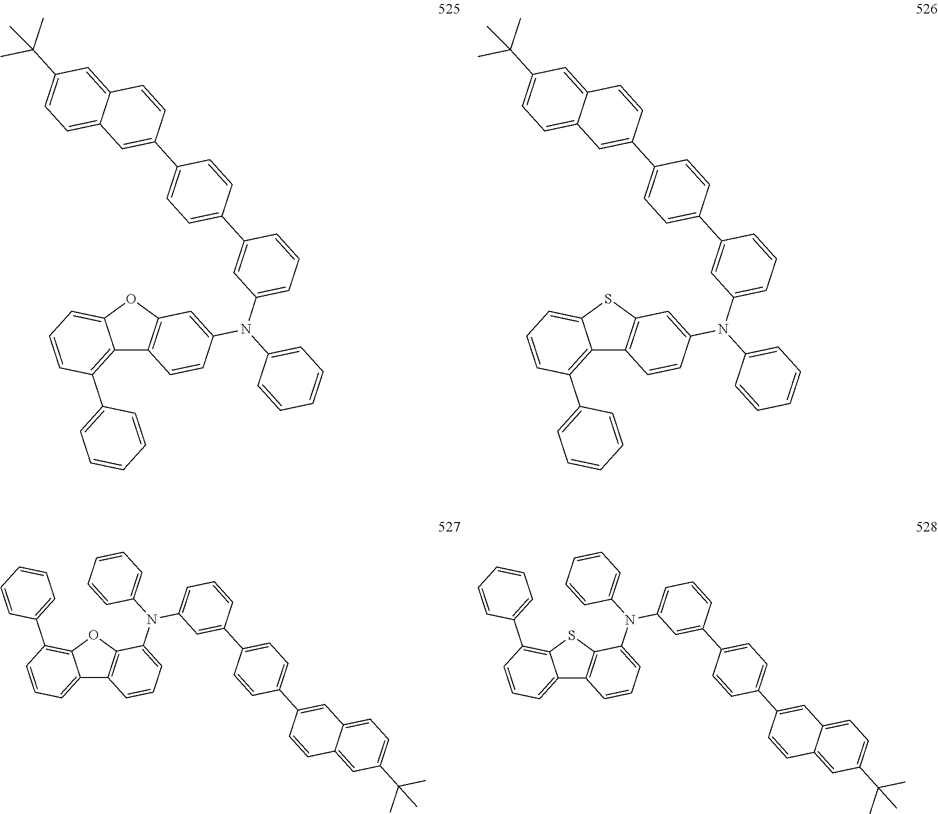 [SMILES: CC(C)(C)c1ccc2cc(-c3ccc(-c4cccc(N(c5ccccc5)c5ccc6c(c5)oc5cccc(-c7ccccc7)c56)c4)cc3)ccc2c1 ; CC(C)(C)c1ccc2cc(-c3ccc(-c4cccc(N(c5ccccc5)c5ccc6c(c5)sc5cccc(-c7ccccc7)c56)c4)cc3)ccc2c1 ; CC(C)(C)c1ccc2cc(-c3ccc(-c4cccc(N(c5ccccc5)c5cccc6c5oc5c(-c7ccccc7)cccc56)c4)cc3)ccc2c1 ; CC(C)(C)c1ccc2cc(-c3ccc(-c4cccc(N(c5ccccc5)c5cccc6c5sc5c(-c7ccccc7)cccc56)c4)cc3)ccc2c1]

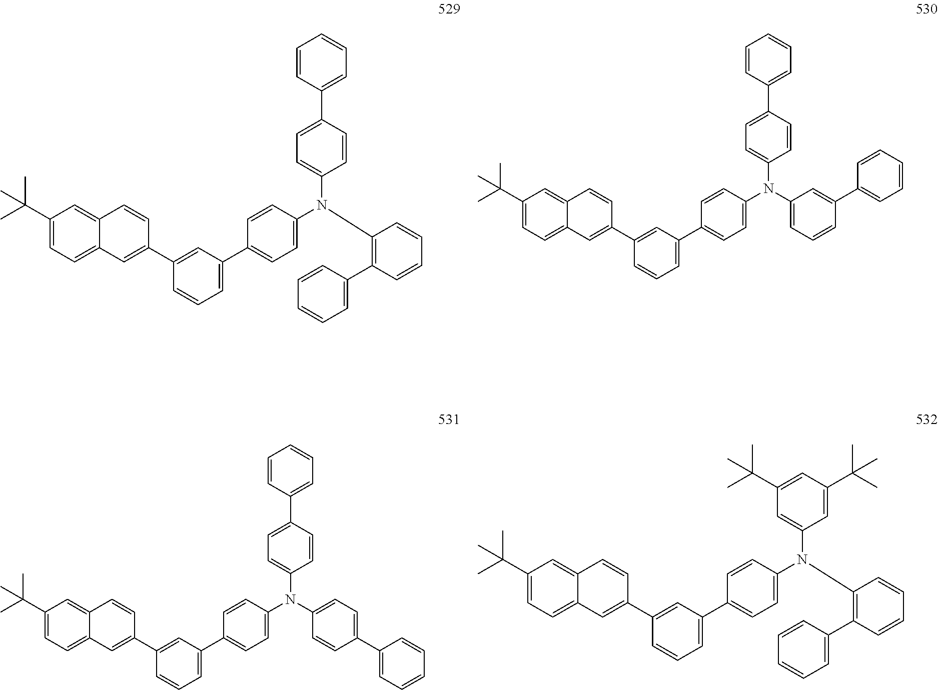 [SMILES: CC(C)(C)c1ccc2cc(-c3cccc(-c4ccc(N(c5ccc(-c6ccccc6)cc5)c5ccccc5-c5ccccc5)cc4)c3)ccc2c1 ; CC(C)(C)c1ccc2cc(-c3cccc(-c4ccc(N(c5ccc(-c6ccccc6)cc5)c5cccc(-c6ccccc6)c5)cc4)c3)ccc2c1 ; CC(C)(C)c1ccc2cc(-c3cccc(-c4ccc(N(c5ccc(-c6ccccc6)cc5)c5ccc(-c6ccccc6)cc5)cc4)c3)ccc2c1 ; CC(C)(C)c1cc(N(c2ccc(-c3cccc(-c4ccc5cc(C(C)(C)C)ccc5c4)c3)cc2)c2ccccc2-c2ccccc2)cc(C(C)(C)C)c1]

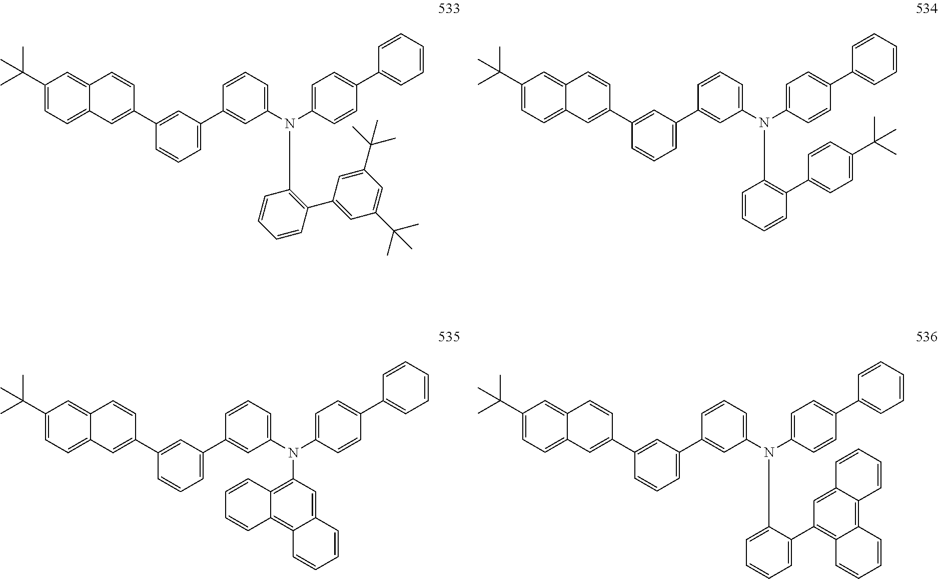 [SMILES: CC(C)(C)c1cc(-c2ccccc2N(c2ccc(-c3ccccc3)cc2)c2cccc(-c3cccc(-c4ccc5cc(C(C)(C)C)ccc5c4)c3)c2)cc(C(C)(C)C)c1 ; CC(C)(C)c1ccc(-c2ccccc2N(c2ccc(-c3ccccc3)cc2)c2cccc(-c3cccc(-c4ccc5cc(C(C)(C)C)ccc5c4)c3)c2)cc1 ; CC(C)(C)c1ccc2cc(-c3cccc(-c4cccc(N(c5ccc(-c6ccccc6)cc5)c5cc6ccccc6c6ccccc56)c4)c3)ccc2c1 ; CC(C)(C)c1ccc2cc(-c3cccc(-c4cccc(N(c5ccc(-c6ccccc6)cc5)c5ccccc5-c5cc6ccccc6c6ccccc56)c4)c3)ccc2c1]

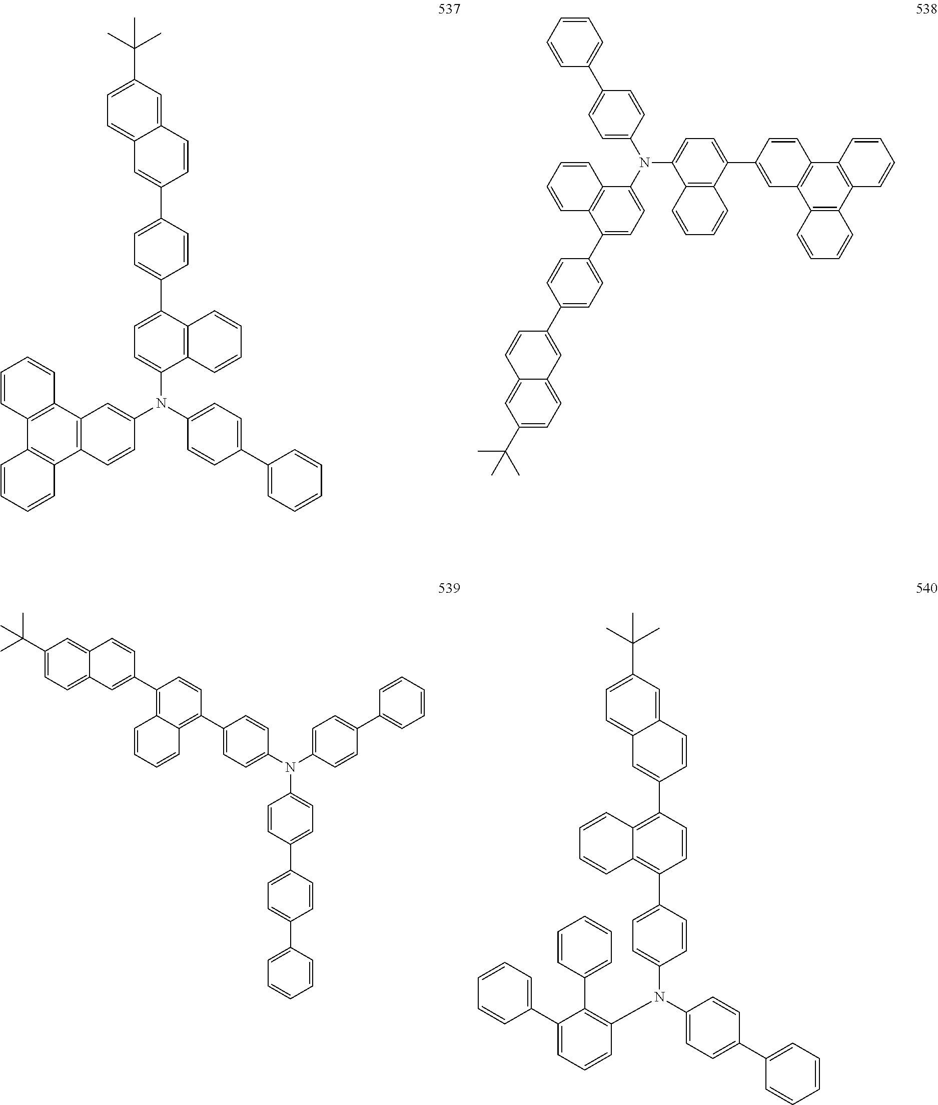 [SMILES: CC(C)(C)c1ccc2cc(-c3ccc(-c4ccc(N(c5ccc(-c6ccccc6)cc5)c5ccc6c7ccccc7c7ccccc7c6c5)c5ccccc45)cc3)ccc2c1 ; CC(C)(C)c1ccc2cc(-c3ccc(-c4ccc(N(c5ccc(-c6ccccc6)cc5)c5ccc(-c6ccc7c8ccccc8c8ccccc8c7c6)c6ccccc56)c5ccccc45)cc3)ccc2c1 ; CC(C)(C)c1ccc2cc(-c3ccc(-c4ccc(N(c5ccc(-c6ccccc6)cc5)c5ccc(-c6ccc(-c7ccccc7)cc6)cc5)cc4)c4ccccc34)ccc2c1 ; CC(C)(C)c1ccc2cc(-c3ccc(-c4ccc(N(c5ccc(-c6ccccc6)cc5)c5cccc(-c6ccccc6)c5-c5ccccc5)cc4)c4ccccc34)ccc2c1]

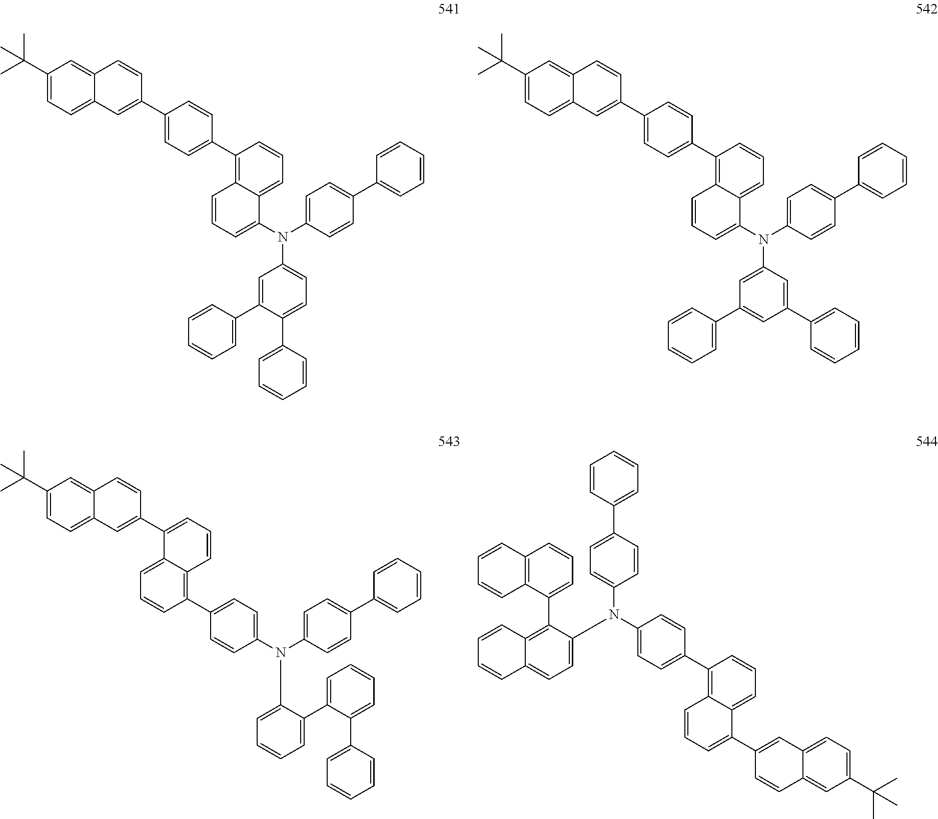 [SMILES: CC(C)(C)c1ccc2cc(-c3ccc(-c4cccc5c(N(c6ccc(-c7ccccc7)cc6)c6ccc(-c7ccccc7)c(-c7ccccc7)c6)cccc45)cc3)ccc2c1 ; CC(C)(C)c1ccc2cc(-c3ccc(-c4cccc5c(N(c6ccc(-c7ccccc7)cc6)c6cc(-c7ccccc7)cc(-c7ccccc7)c6)cccc45)cc3)ccc2c1 ; CC(C)(C)c1ccc2cc(-c3cccc4c(-c5ccc(N(c6ccc(-c7ccccc7)cc6)c6ccccc6-c6ccccc6-c6ccccc6)cc5)cccc34)ccc2c1 ; CC(C)(C)c1ccc2cc(-c3cccc4c(-c5ccc(N(c6ccc(-c7ccccc7)cc6)c6ccc7ccccc7c6-c6cccc7ccccc67)cc5)cccc34)ccc2c1]

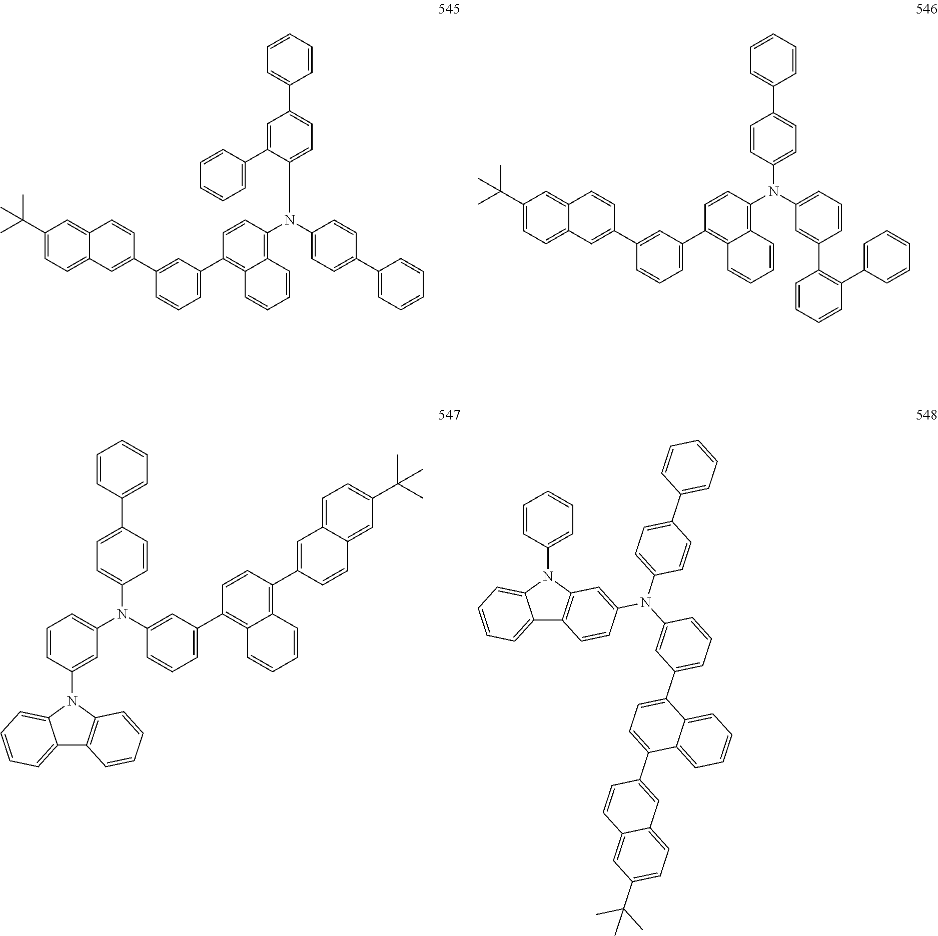 [SMILES: CC(C)(C)c1ccc2cc(-c3cccc(-c4ccc(N(c5ccc(-c6ccccc6)cc5)c5ccc(-c6ccccc6)cc5-c5ccccc5)c5ccccc45)c3)ccc2c1 ; CC(C)(C)c1ccc2cc(-c3cccc(-c4ccc(N(c5ccc(-c6ccccc6)cc5)c5cccc(-c6ccccc6-c6ccccc6)c5)c5ccccc45)c3)ccc2c1 ; CC(C)(C)c1ccc2cc(-c3ccc(-c4cccc(N(c5ccc(-c6ccccc6)cc5)c5cccc(-n6c7ccccc7c7ccccc76)c5)c4)c4ccccc34)ccc2c1 ; CC(C)(C)c1ccc2cc(-c3ccc(-c4cccc(N(c5ccc(-c6ccccc6)cc5)c5ccc6c7ccccc7n(-c7ccccc7)c6c5)c4)c4ccccc34)ccc2c1]

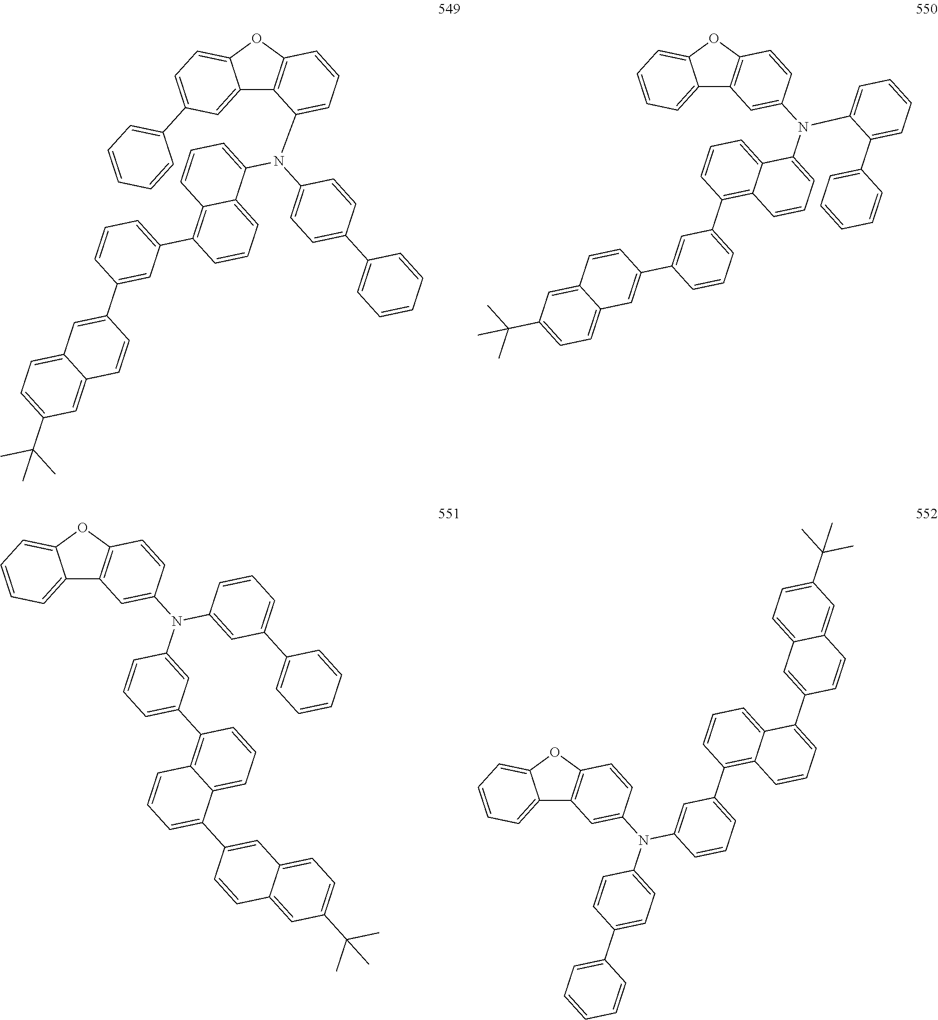 [SMILES: CC(C)(C)c1ccc2cc(-c3cccc(-c4cccc5c(N(c6ccc(-c7ccccc7)cc6)c6cccc7oc8ccc(-c9ccccc9)cc8c67)cccc45)c3)ccc2c1 ; CC(C)(C)c1ccc2cc(-c3cccc(-c4cccc5c(N(c6ccc7oc8ccccc8c7c6)c6ccccc6-c6ccccc6)cccc45)c3)ccc2c1 ; CC(C)(C)c1ccc2cc(-c3cccc4c(-c5cccc(N(c6cccc(-c7ccccc7)c6)c6ccc7oc8ccccc8c7c6)c5)cccc34)ccc2c1 ; CC(C)(C)c1ccc2cc(-c3cccc4c(-c5cccc(N(c6ccc(-c7ccccc7)cc6)c6ccc7oc8ccccc8c7c6)c5)cccc34)ccc2c1]

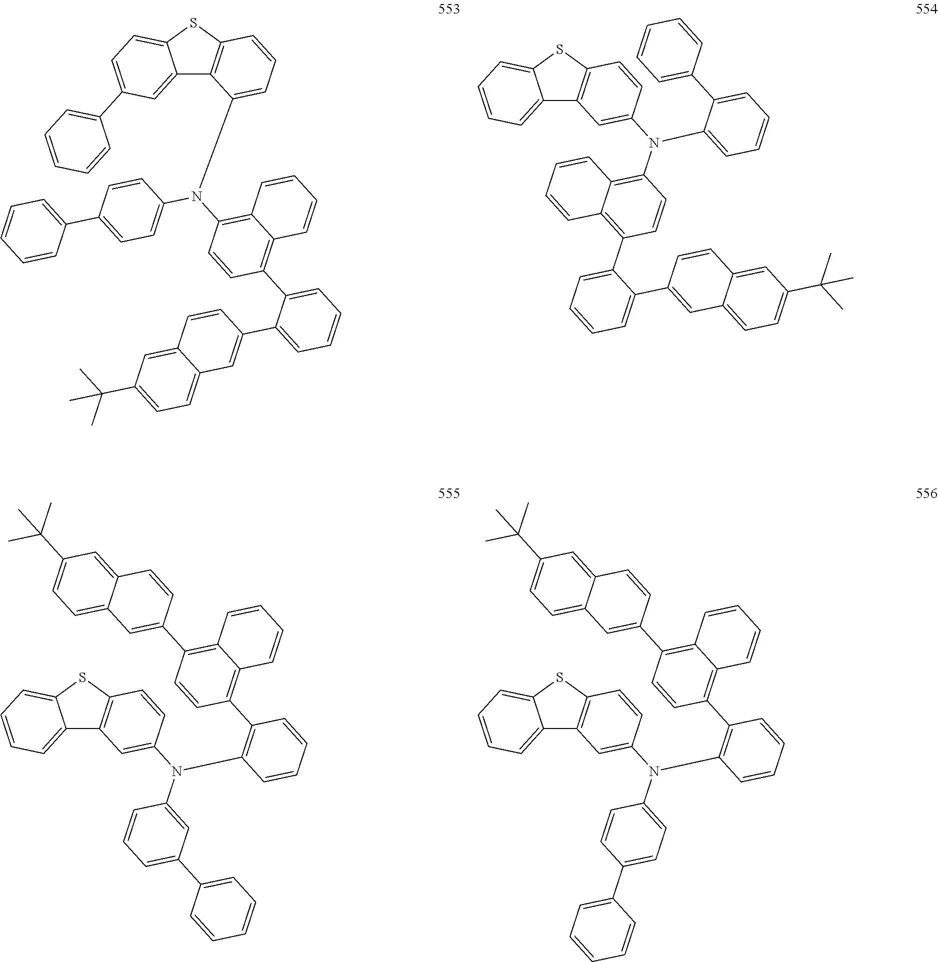 [SMILES: CC(C)(C)c1ccc2cc(-c3ccccc3-c3ccc(N(c4ccc(-c5ccccc5)cc4)c4cccc5sc6ccc(-c7ccccc7)cc6c45)c4ccccc34)ccc2c1 ; CC(C)(C)c1ccc2cc(-c3ccccc3-c3ccc(N(c4ccc5sc6ccccc6c5c4)c4ccccc4-c4ccccc4)c4ccccc34)ccc2c1 ; CC(C)(C)c1ccc2cc(-c3ccc(-c4ccccc4N(c4cccc(-c5ccccc5)c4)c4ccc5sc6ccccc6c5c4)c4ccccc34)ccc2c1 ; CC(C)(C)c1ccc2cc(-c3ccc(-c4ccccc4N(c4ccc(-c5ccccc5)cc4)c4ccc5sc6ccccc6c5c4)c4ccccc34)ccc2c1]

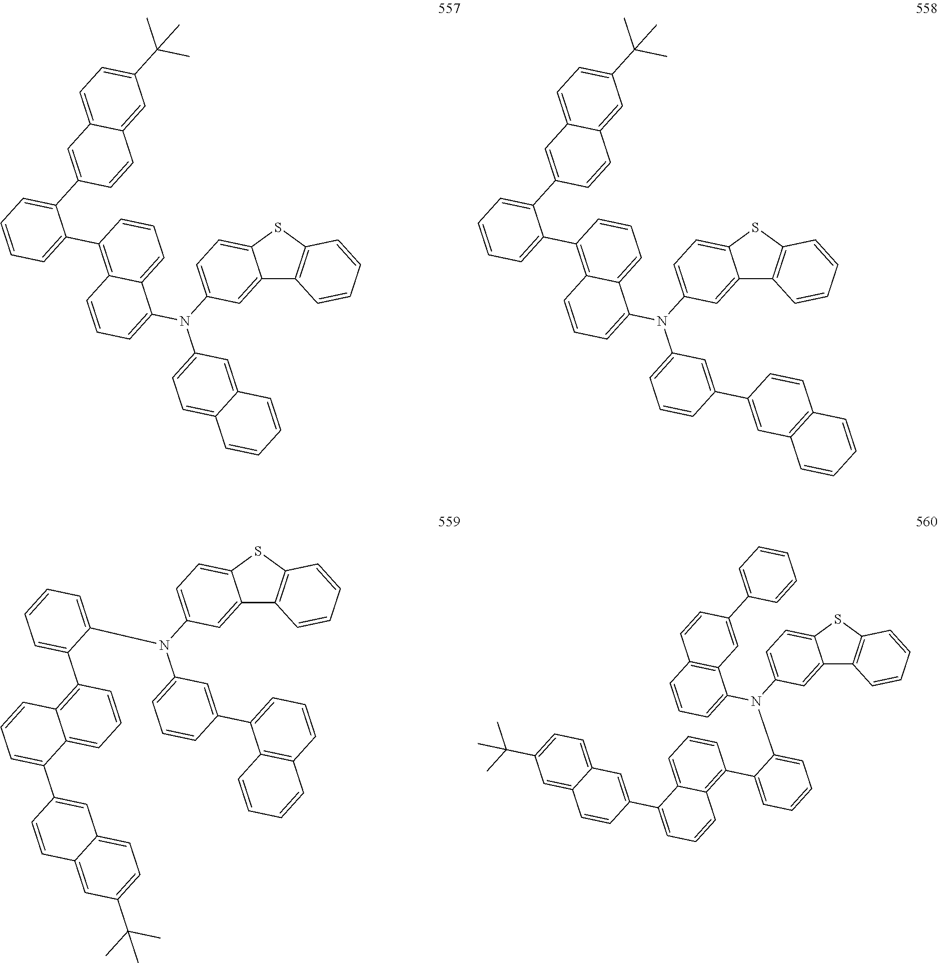 [SMILES: CC(C)(C)c1ccc2cc(-c3ccccc3-c3cccc4c(N(c5ccc6ccccc6c5)c5ccc6sc7ccccc7c6c5)cccc34)ccc2c1 ; CC(C)(C)c1ccc2cc(-c3ccccc3-c3cccc4c(N(c5cccc(-c6ccc7ccccc7c6)c5)c5ccc6sc7ccccc7c6c5)cccc34)ccc2c1 ; CC(C)(C)c1ccc2cc(-c3cccc4c(-c5ccccc5N(c5cccc(-c6cccc7ccccc67)c5)c5ccc6sc7ccccc7c6c5)cccc34)ccc2c1 ; CC(C)(C)c1ccc2cc(-c3cccc4c(-c5ccccc5N(c5ccc6sc7ccccc7c6c5)c5cccc6ccc(-c7ccccc7)cc56)cccc34)ccc2c1]

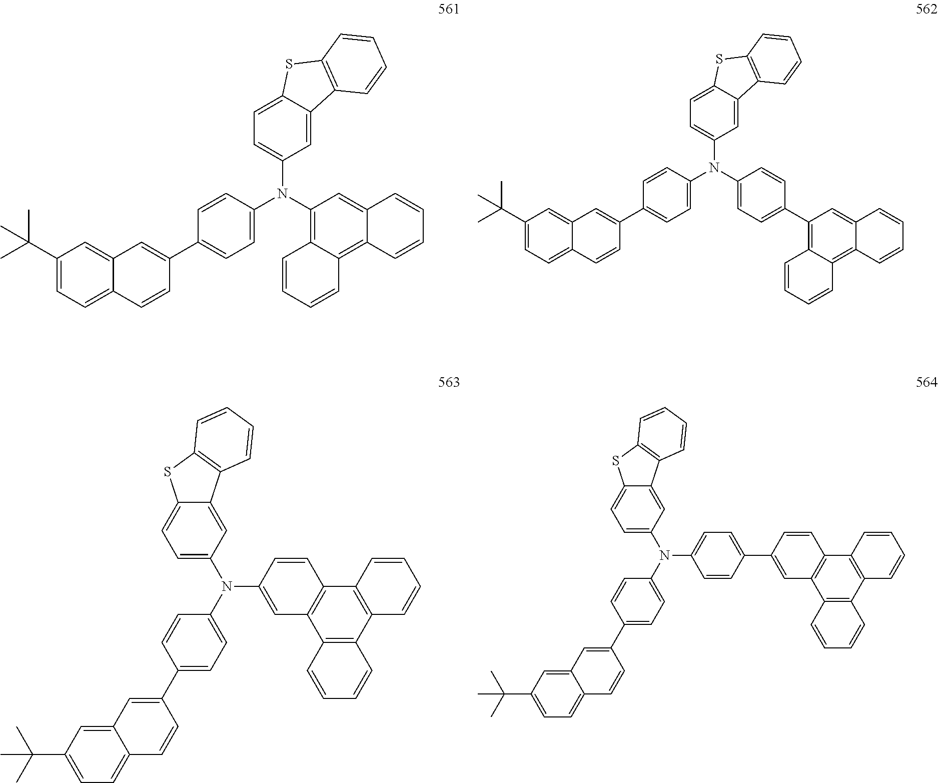 [SMILES: CC(C)(C)c1ccc2ccc(-c3ccc(N(c4ccc5sc6ccccc6c5c4)c4cc5ccccc5c5ccccc45)cc3)cc2c1 ; CC(C)(C)c1ccc2ccc(-c3ccc(N(c4ccc(-c5cc6ccccc6c6ccccc56)cc4)c4ccc5sc6ccccc6c5c4)cc3)cc2c1 ; CC(C)(C)c1ccc2ccc(-c3ccc(N(c4ccc5sc6ccccc6c5c4)c4ccc5c6ccccc6c6ccccc6c5c4)cc3)cc2c1 ; CC(C)(C)c1ccc2ccc(-c3ccc(N(c4ccc(-c5ccc6c7ccccc7c7ccccc7c6c5)cc4)c4ccc5sc6ccccc6c5c4)cc3)cc2c1]

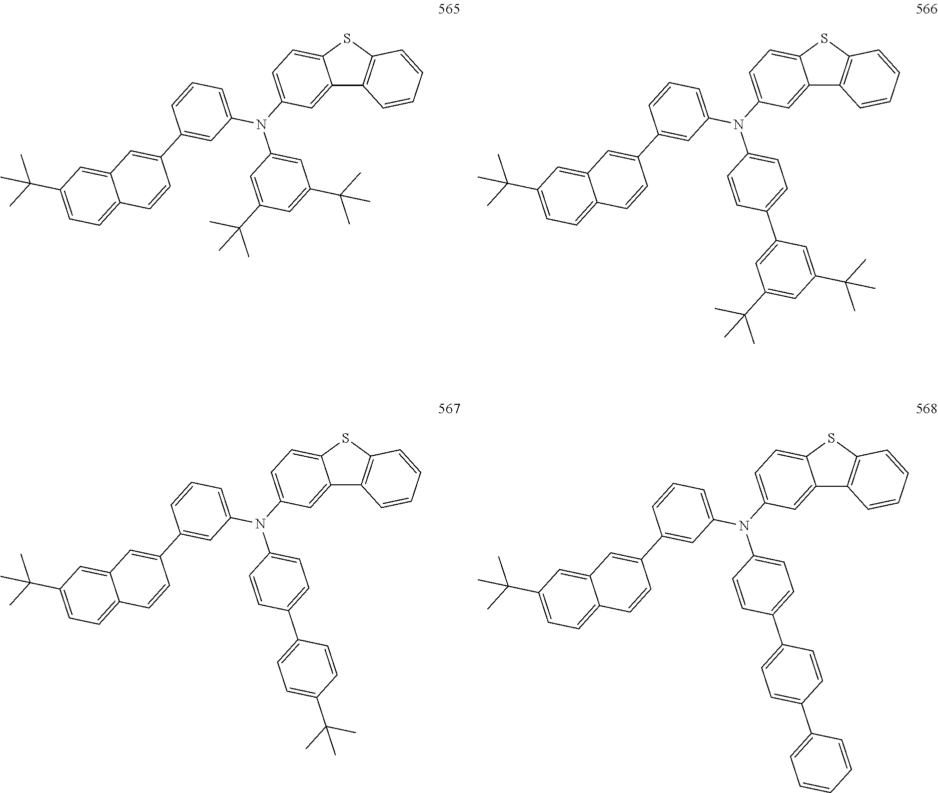 [SMILES: CC(C)(C)c1cc(N(c2cccc(-c3ccc4ccc(C(C)(C)C)cc4c3)c2)c2ccc3sc4ccccc4c3c2)cc(C(C)(C)C)c1 ; CC(C)(C)c1cc(-c2ccc(N(c3cccc(-c4ccc5ccc(C(C)(C)C)cc5c4)c3)c3ccc4sc5ccccc5c4c3)cc2)cc(C(C)(C)C)c1 ; CC(C)(C)c1ccc(-c2ccc(N(c3cccc(-c4ccc5ccc(C(C)(C)C)cc5c4)c3)c3ccc4sc5ccccc5c4c3)cc2)cc1 ; CC(C)(C)c1ccc2ccc(-c3cccc(N(c4ccc(-c5ccc(-c6ccccc6)cc5)cc4)c4ccc5sc6ccccc6c5c4)c3)cc2c1]

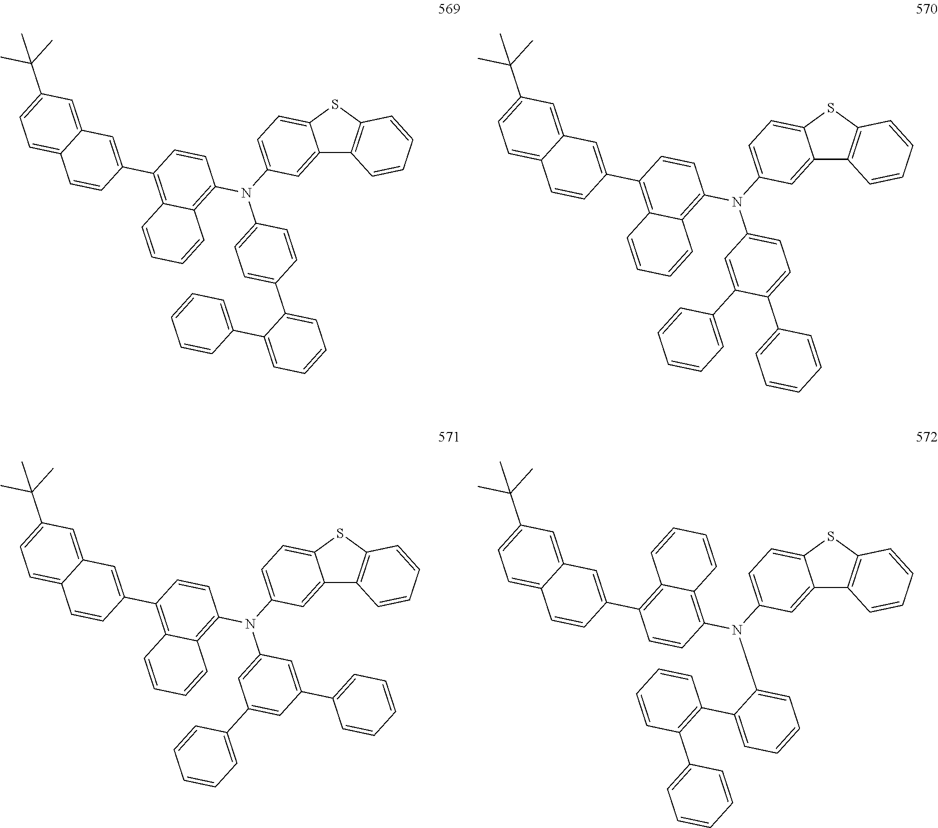 [SMILES: CC(C)(C)c1ccc2ccc(-c3ccc(N(c4ccc(-c5ccccc5-c5ccccc5)cc4)c4ccc5sc6ccccc6c5c4)c4ccccc34)cc2c1 ; CC(C)(C)c1ccc2ccc(-c3ccc(N(c4ccc(-c5ccccc5)c(-c5ccccc5)c4)c4ccc5sc6ccccc6c5c4)c4ccccc34)cc2c1 ; CC(C)(C)c1ccc2ccc(-c3ccc(N(c4cc(-c5ccccc5)cc(-c5ccccc5)c4)c4ccc5sc6ccccc6c5c4)c4ccccc34)cc2c1 ; CC(C)(C)c1ccc2ccc(-c3ccc(N(c4ccc5sc6ccccc6c5c4)c4ccccc4-c4ccccc4-c4ccccc4)c4ccccc34)cc2c1]

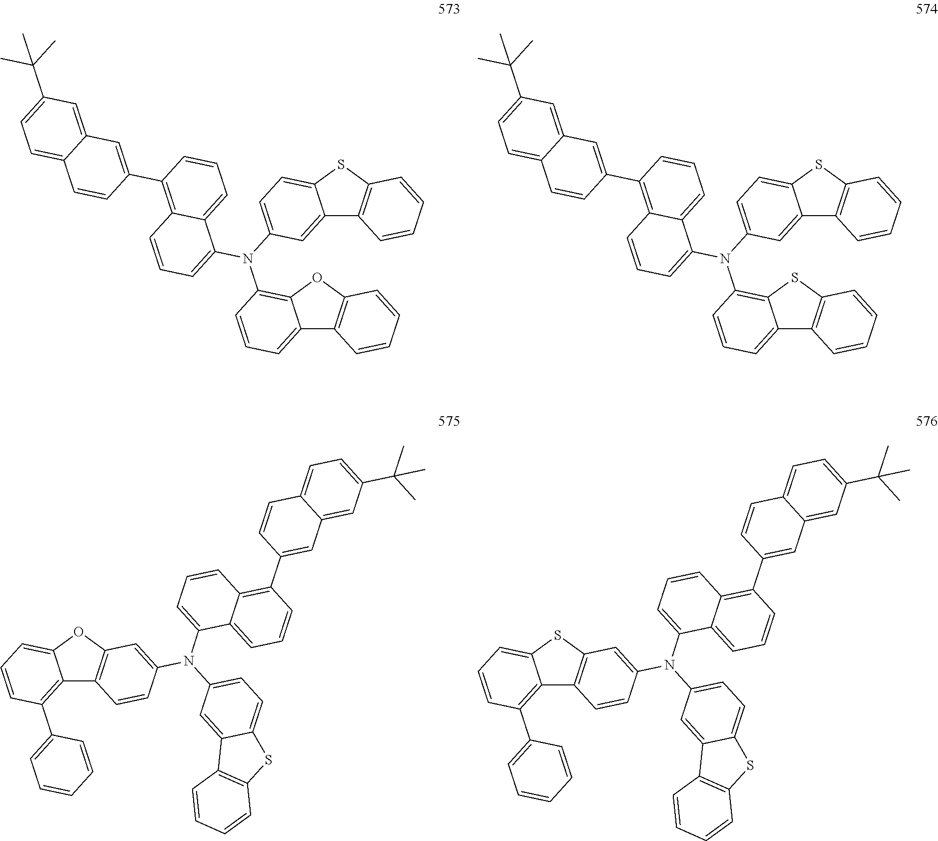 [SMILES: CC(C)(C)c1ccc2ccc(-c3cccc4c(N(c5ccc6sc7ccccc7c6c5)c5cccc6c5oc5ccccc56)cccc34)cc2c1 ; CC(C)(C)c1ccc2ccc(-c3cccc4c(N(c5ccc6sc7ccccc7c6c5)c5cccc6c5sc5ccccc56)cccc34)cc2c1 ; CC(C)(C)c1ccc2ccc(-c3cccc4c(N(c5ccc6c(c5)oc5cccc(-c7ccccc7)c56)c5ccc6sc7ccccc7c6c5)cccc34)cc2c1 ; CC(C)(C)c1ccc2ccc(-c3cccc4c(N(c5ccc6c(c5)sc5cccc(-c7ccccc7)c56)c5ccc6sc7ccccc7c6c5)cccc34)cc2c1]

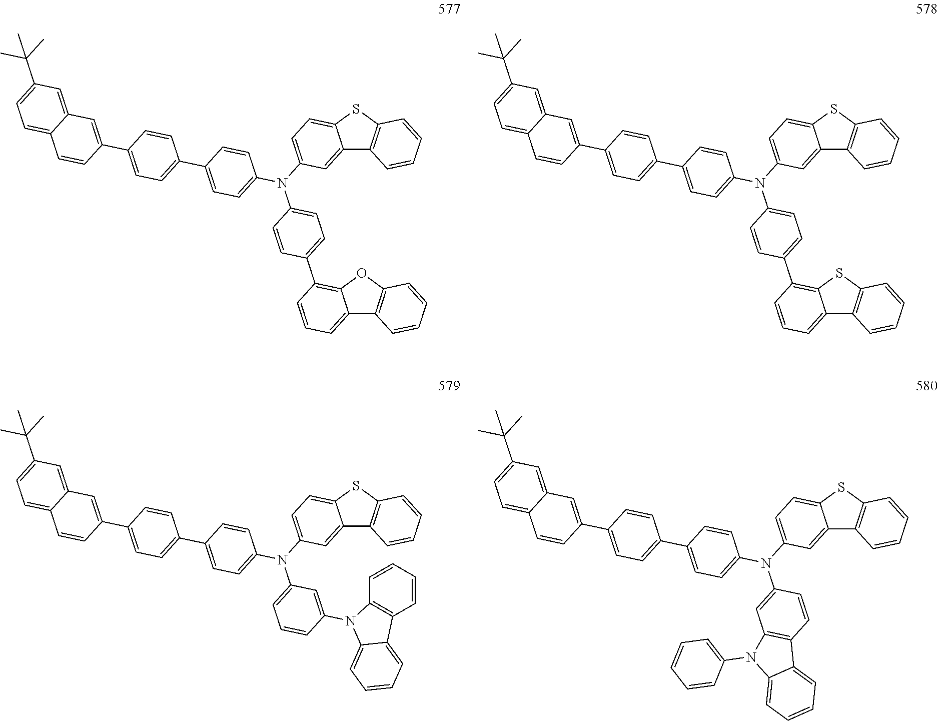 [SMILES: CC(C)(C)c1ccc2ccc(-c3ccc(-c4ccc(N(c5ccc(-c6cccc7c6oc6ccccc67)cc5)c5ccc6sc7ccccc7c6c5)cc4)cc3)cc2c1 ; CC(C)(C)c1ccc2ccc(-c3ccc(-c4ccc(N(c5ccc(-c6cccc7c6sc6ccccc67)cc5)c5ccc6sc7ccccc7c6c5)cc4)cc3)cc2c1 ; CC(C)(C)c1ccc2ccc(-c3ccc(-c4ccc(N(c5cccc(-n6c7ccccc7c7ccccc76)c5)c5ccc6sc7ccccc7c6c5)cc4)cc3)cc2c1 ; CC(C)(C)c1ccc2ccc(-c3ccc(-c4ccc(N(c5ccc6sc7ccccc7c6c5)c5ccc6c7ccccc7n(-c7ccccc7)c6c5)cc4)cc3)cc2c1]

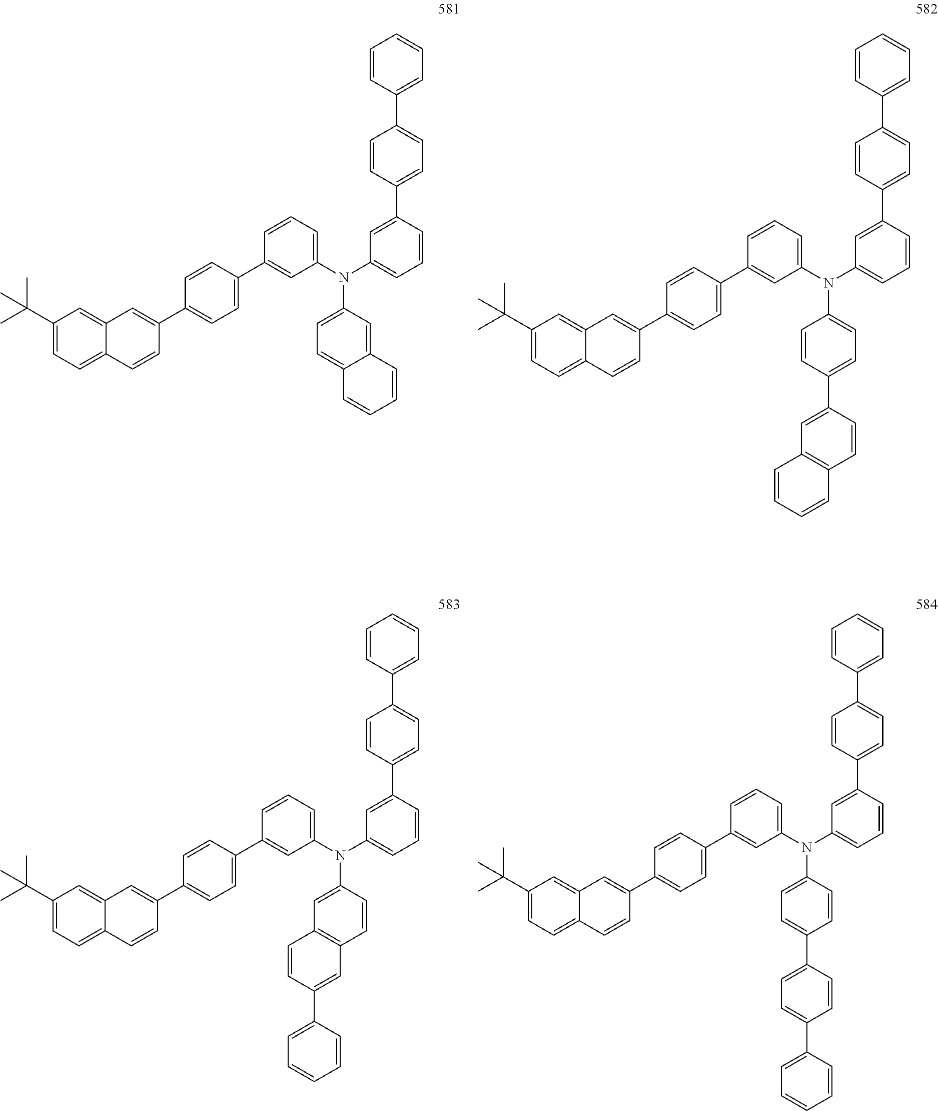 [SMILES: CC(C)(C)c1ccc2ccc(-c3ccc(-c4cccc(N(c5cccc(-c6ccc(-c7ccccc7)cc6)c5)c5ccc6ccccc6c5)c4)cc3)cc2c1 ; CC(C)(C)c1ccc2ccc(-c3ccc(-c4cccc(N(c5ccc(-c6ccc7ccccc7c6)cc5)c5cccc(-c6ccc(-c7ccccc7)cc6)c5)c4)cc3)cc2c1 ; CC(C)(C)c1ccc2ccc(-c3ccc(-c4cccc(N(c5cccc(-c6ccc(-c7ccccc7)cc6)c5)c5ccc6cc(-c7ccccc7)ccc6c5)c4)cc3)cc2c1 ; CC(C)(C)c1ccc2ccc(-c3ccc(-c4cccc(N(c5ccc(-c6ccc(-c7ccccc7)cc6)cc5)c5cccc(-c6ccc(-c7ccccc7)cc6)c5)c4)cc3)cc2c1]

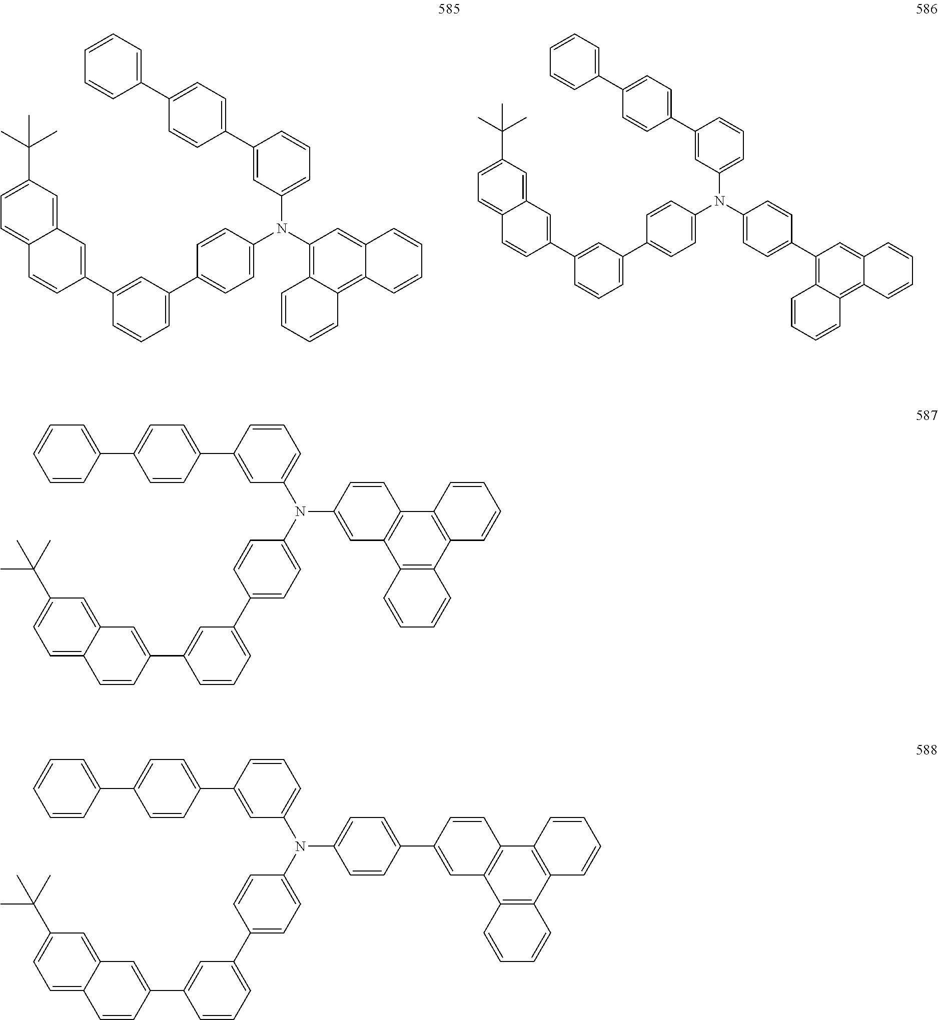 [SMILES: CC(C)(C)c1ccc2ccc(-c3cccc(-c4ccc(N(c5cccc(-c6ccc(-c7ccccc7)cc6)c5)c5cc6ccccc6c6ccccc56)cc4)c3)cc2c1 ; CC(C)(C)c1ccc2ccc(-c3cccc(-c4ccc(N(c5ccc(-c6cc7ccccc7c7ccccc67)cc5)c5cccc(-c6ccc(-c7ccccc7)cc6)c5)cc4)c3)cc2c1 ; CC(C)(C)c1ccc2ccc(-c3cccc(-c4ccc(N(c5cccc(-c6ccc(-c7ccccc7)cc6)c5)c5ccc6c7ccccc7c7ccccc7c6c5)cc4)c3)cc2c1 ; CC(C)(C)c1ccc2ccc(-c3cccc(-c4ccc(N(c5ccc(-c6ccc7c8ccccc8c8ccccc8c7c6)cc5)c5cccc(-c6ccc(-c7ccccc7)cc6)c5)cc4)c3)cc2c1]

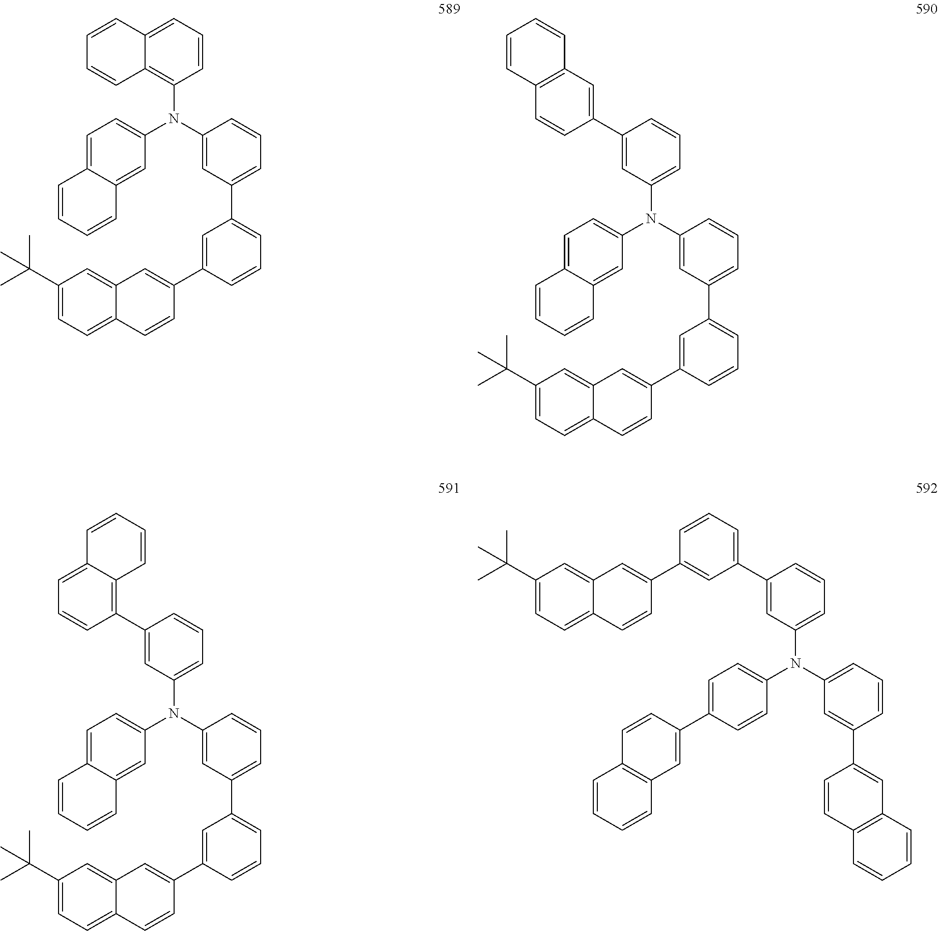 [SMILES: CC(C)(C)c1ccc2ccc(-c3cccc(-c4cccc(N(c5ccc6ccccc6c5)c5cccc6ccccc56)c4)c3)cc2c1 ; CC(C)(C)c1ccc2ccc(-c3cccc(-c4cccc(N(c5cccc(-c6ccc7ccccc7c6)c5)c5ccc6ccccc6c5)c4)c3)cc2c1 ; CC(C)(C)c1ccc2ccc(-c3cccc(-c4cccc(N(c5cccc(-c6cccc7ccccc67)c5)c5ccc6ccccc6c5)c4)c3)cc2c1 ; CC(C)(C)c1ccc2ccc(-c3cccc(-c4cccc(N(c5ccc(-c6ccc7ccccc7c6)cc5)c5cccc(-c6ccc7ccccc7c6)c5)c4)c3)cc2c1]

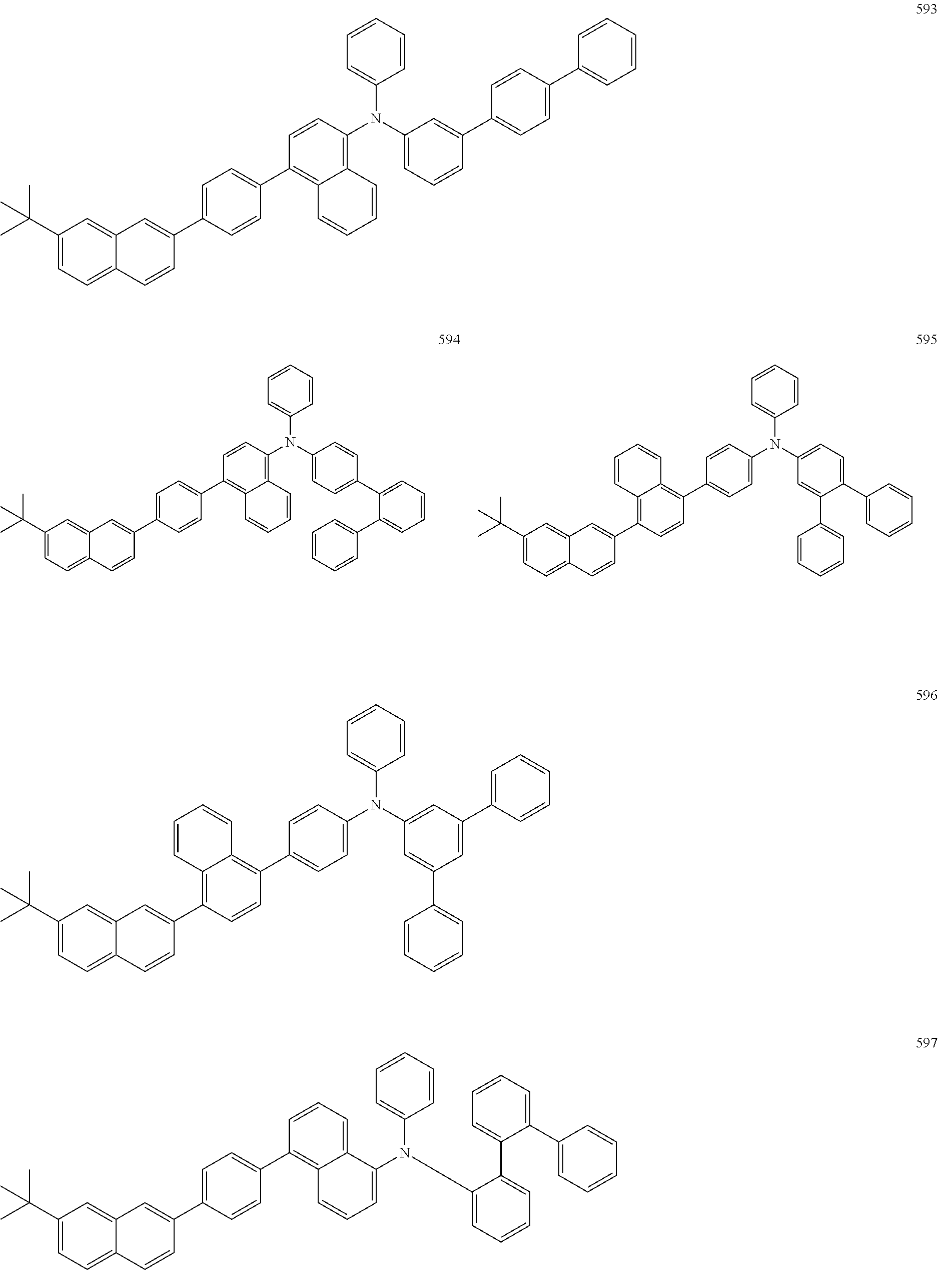 [SMILES: CC(C)(C)c1ccc2ccc(-c3ccc(-c4ccc(N(c5ccccc5)c5cccc(-c6ccc(-c7ccccc7)cc6)c5)c5ccccc45)cc3)cc2c1 ; CC(C)(C)c1ccc2ccc(-c3ccc(-c4ccc(N(c5ccccc5)c5ccc(-c6ccccc6-c6ccccc6)cc5)c5ccccc45)cc3)cc2c1 ; CC(C)(C)c1ccc2ccc(-c3ccc(-c4ccc(N(c5ccccc5)c5ccc(-c6ccccc6)c(-c6ccccc6)c5)cc4)c4ccccc34)cc2c1 ; CC(C)(C)c1ccc2ccc(-c3ccc(-c4ccc(N(c5ccccc5)c5cc(-c6ccccc6)cc(-c6ccccc6)c5)cc4)c4ccccc34)cc2c1 ; CC(C)(C)c1ccc2ccc(-c3ccc(-c4cccc5c(N(c6ccccc6)c6ccccc6-c6ccccc6-c6ccccc6)cccc45)cc3)cc2c1]

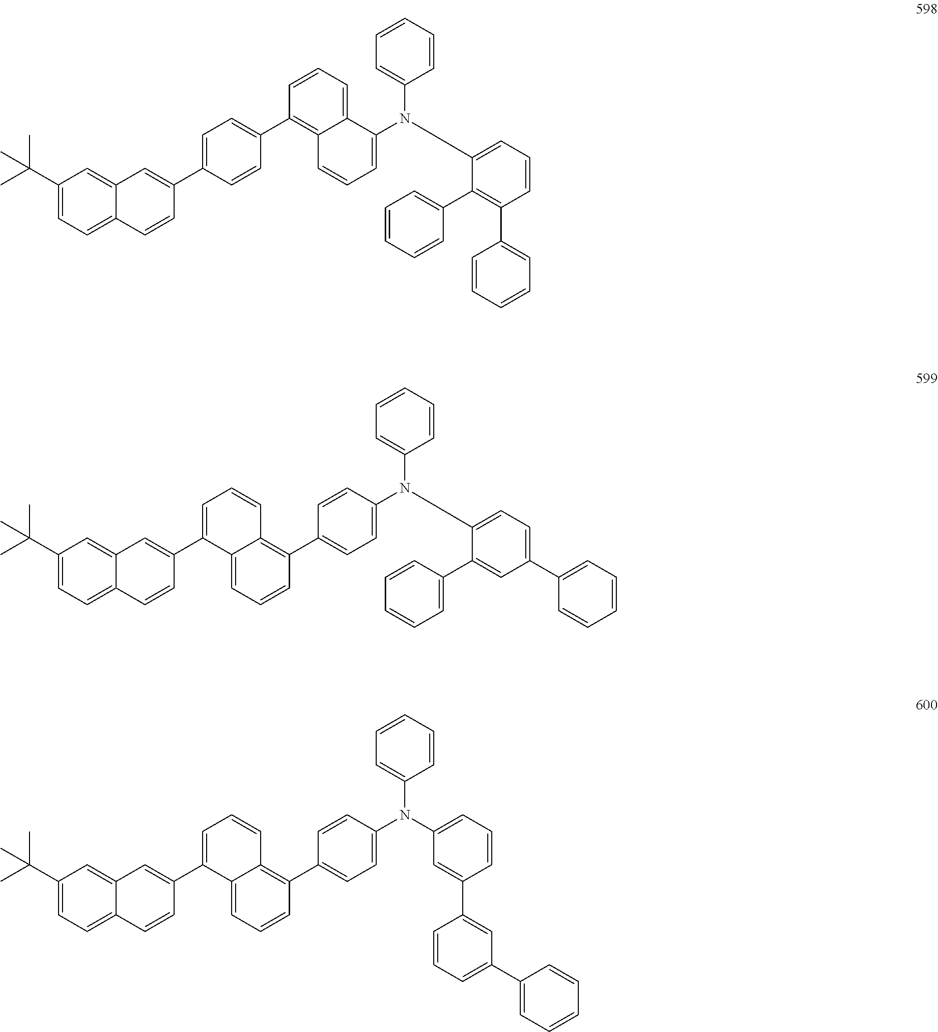 [SMILES: CC(C)(C)c1ccc2ccc(-c3ccc(-c4cccc5c(N(c6ccccc6)c6cccc(-c7ccccc7)c6-c6ccccc6)cccc45)cc3)cc2c1 ; CC(C)(C)c1ccc2ccc(-c3cccc4c(-c5ccc(N(c6ccccc6)c6ccc(-c7ccccc7)cc6-c6ccccc6)cc5)cccc34)cc2c1 ; CC(C)(C)c1ccc2ccc(-c3cccc4c(-c5ccc(N(c6ccccc6)c6cccc(-c7cccc(-c8ccccc8)c7)c6)cc5)cccc34)cc2c1]

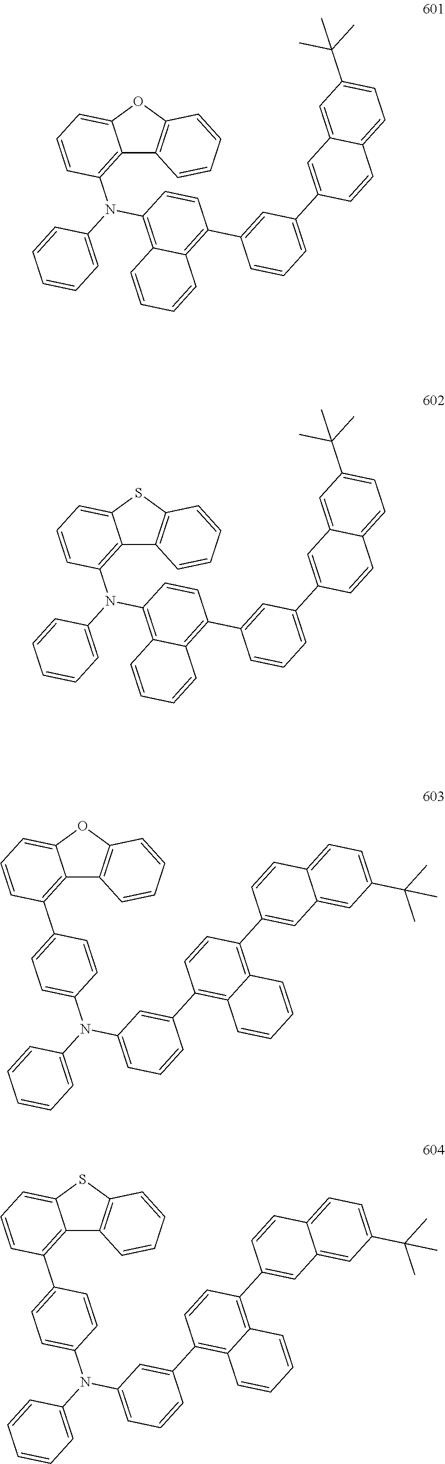 [SMILES: CC(C)(C)c1ccc2ccc(-c3cccc(-c4ccc(N(c5ccccc5)c5cccc6oc7ccccc7c56)c5ccccc45)c3)cc2c1 ; CC(C)(C)c1ccc2ccc(-c3cccc(-c4ccc(N(c5ccccc5)c5cccc6sc7ccccc7c56)c5ccccc45)c3)cc2c1 ; CC(C)(C)c1ccc2ccc(-c3ccc(-c4cccc(N(c5ccccc5)c5ccc(-c6cccc7oc8ccccc8c67)cc5)c4)c4ccccc34)cc2c1 ; CC(C)(C)c1ccc2ccc(-c3ccc(-c4cccc(N(c5ccccc5)c5ccc(-c6cccc7sc8ccccc8c67)cc5)c4)c4ccccc34)cc2c1]

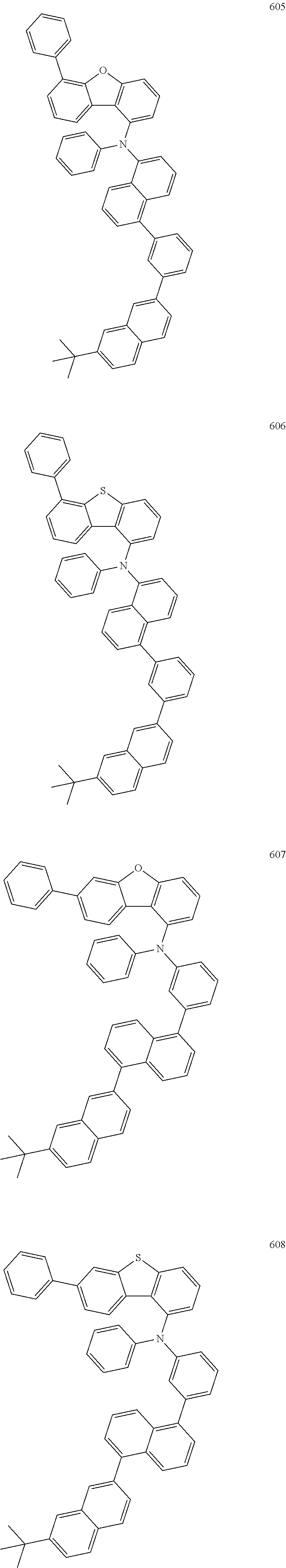 [SMILES: CC(C)(C)c1ccc2ccc(-c3cccc(-c4cccc5c(N(c6ccccc6)c6cccc7oc8c(-c9ccccc9)cccc8c67)cccc45)c3)cc2c1 ; CC(C)(C)c1ccc2ccc(-c3cccc(-c4cccc5c(N(c6ccccc6)c6cccc7sc8c(-c9ccccc9)cccc8c67)cccc45)c3)cc2c1 ; CC(C)(C)c1ccc2ccc(-c3cccc4c(-c5cccc(N(c6ccccc6)c6cccc7oc8cc(-c9ccccc9)ccc8c67)c5)cccc34)cc2c1 ; CC(C)(C)c1ccc2ccc(-c3cccc4c(-c5cccc(N(c6ccccc6)c6cccc7sc8cc(-c9ccccc9)ccc8c67)c5)cccc34)cc2c1]

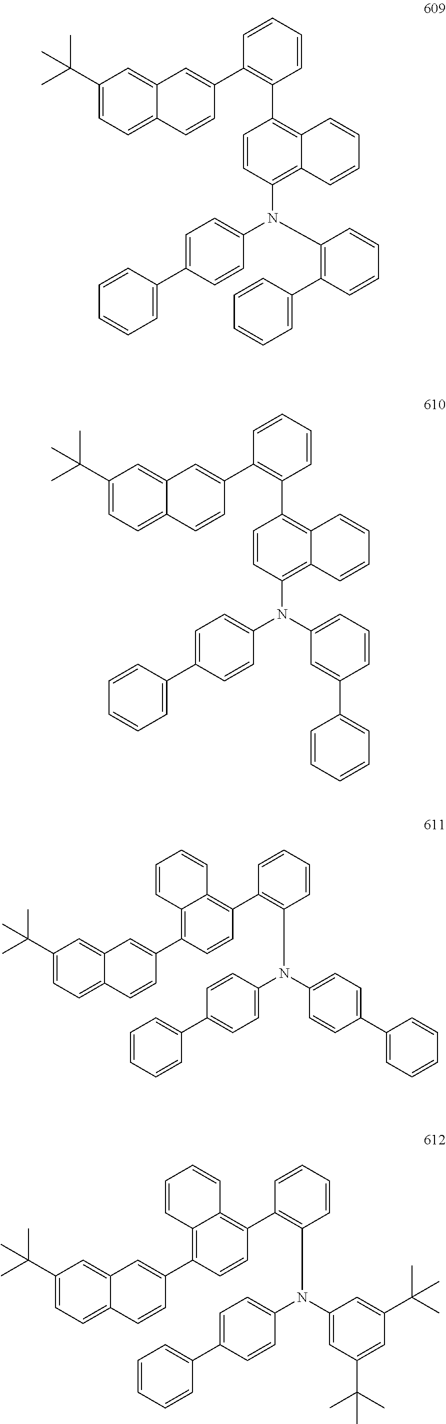 [SMILES: CC(C)(C)c1ccc2ccc(-c3ccccc3-c3ccc(N(c4ccc(-c5ccccc5)cc4)c4ccccc4-c4ccccc4)c4ccccc34)cc2c1 ; CC(C)(C)c1ccc2ccc(-c3ccccc3-c3ccc(N(c4ccc(-c5ccccc5)cc4)c4cccc(-c5ccccc5)c4)c4ccccc34)cc2c1 ; CC(C)(C)c1ccc2ccc(-c3ccc(-c4ccccc4N(c4ccc(-c5ccccc5)cc4)c4ccc(-c5ccccc5)cc4)c4ccccc34)cc2c1 ; CC(C)(C)c1cc(N(c2ccc(-c3ccccc3)cc2)c2ccccc2-c2ccc(-c3ccc4ccc(C(C)(C)C)cc4c3)c3ccccc23)cc(C(C)(C)C)c1]

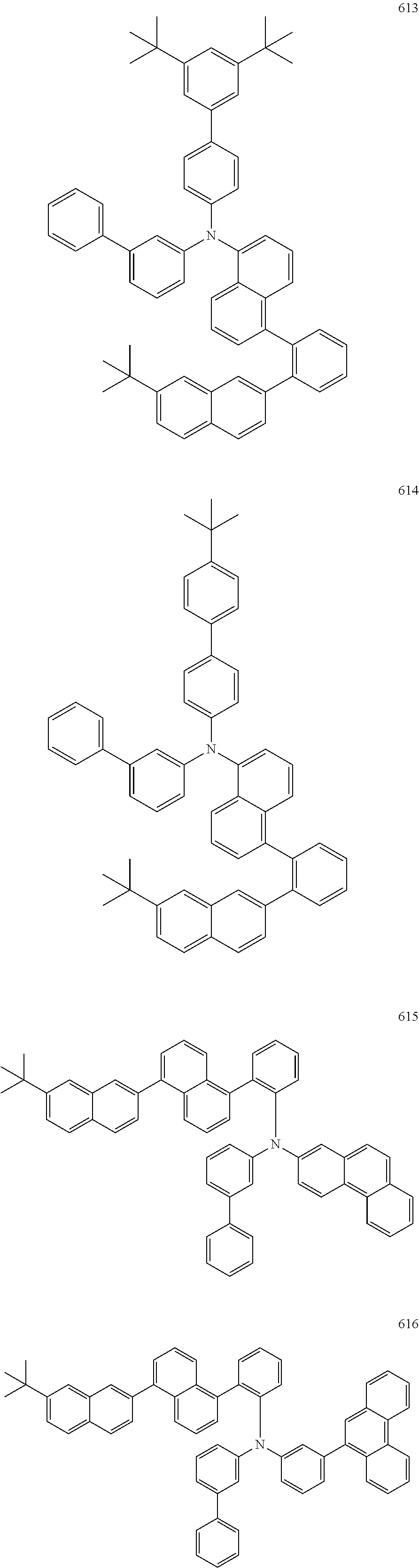 [SMILES: CC(C)(C)c1cc(-c2ccc(N(c3cccc(-c4ccccc4)c3)c3cccc4c(-c5ccccc5-c5ccc6ccc(C(C)(C)C)cc6c5)cccc34)cc2)cc(C(C)(C)C)c1 ; CC(C)(C)c1ccc(-c2ccc(N(c3cccc(-c4ccccc4)c3)c3cccc4c(-c5ccccc5-c5ccc6ccc(C(C)(C)C)cc6c5)cccc34)cc2)cc1 ; CC(C)(C)c1ccc2ccc(-c3cccc4c(-c5ccccc5N(c5cccc(-c6ccccc6)c5)c5ccc6c(ccc7ccccc76)c5)cccc34)cc2c1 ; CC(C)(C)c1ccc2ccc(-c3cccc4c(-c5ccccc5N(c5cccc(-c6ccccc6)c5)c5cccc(-c6cc7ccccc7c7ccccc67)c5)cccc34)cc2c1]

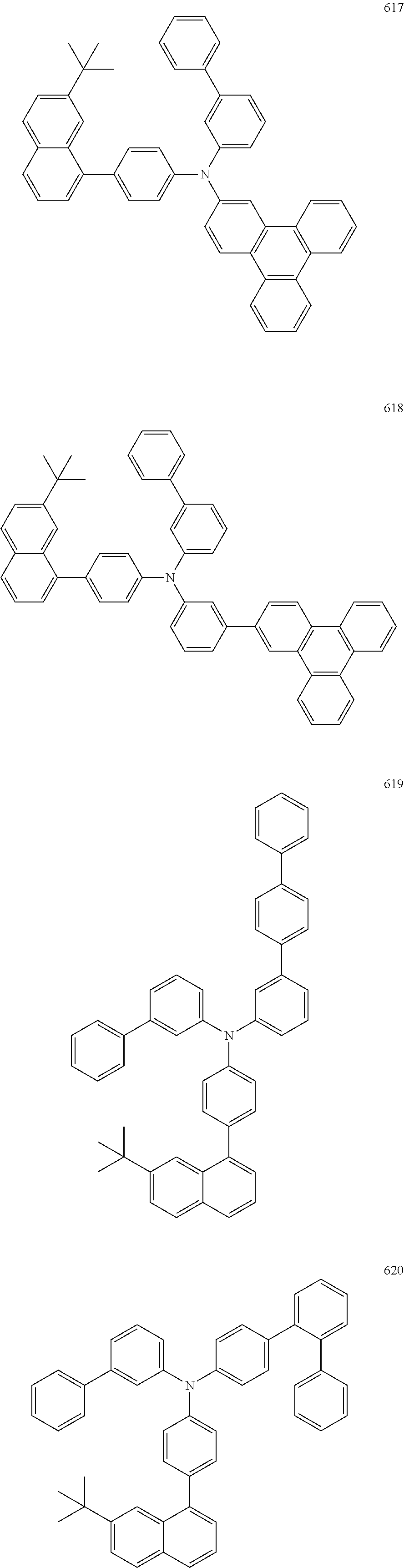 [SMILES: CC(C)(C)c1ccc2cccc(-c3ccc(N(c4cccc(-c5ccccc5)c4)c4ccc5c6ccccc6c6ccccc6c5c4)cc3)c2c1 ; CC(C)(C)c1ccc2cccc(-c3ccc(N(c4cccc(-c5ccccc5)c4)c4cccc(-c5ccc6c7ccccc7c7ccccc7c6c5)c4)cc3)c2c1 ; CC(C)(C)c1ccc2cccc(-c3ccc(N(c4cccc(-c5ccccc5)c4)c4cccc(-c5ccc(-c6ccccc6)cc5)c4)cc3)c2c1 ; CC(C)(C)c1ccc2cccc(-c3ccc(N(c4ccc(-c5ccccc5-c5ccccc5)cc4)c4cccc(-c5ccccc5)c4)cc3)c2c1]

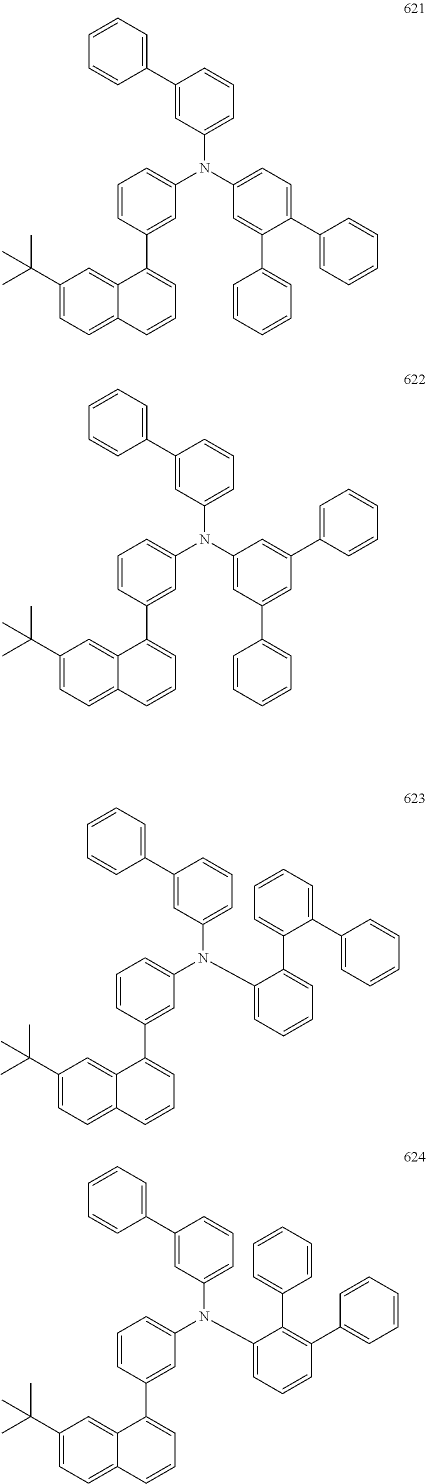 [SMILES: CC(C)(C)c1ccc2cccc(-c3cccc(N(c4cccc(-c5ccccc5)c4)c4ccc(-c5ccccc5)c(-c5ccccc5)c4)c3)c2c1 ; CC(C)(C)c1ccc2cccc(-c3cccc(N(c4cccc(-c5ccccc5)c4)c4cc(-c5ccccc5)cc(-c5ccccc5)c4)c3)c2c1 ; CC(C)(C)c1ccc2cccc(-c3cccc(N(c4cccc(-c5ccccc5)c4)c4ccccc4-c4ccccc4-c4ccccc4)c3)c2c1 ; CC(C)(C)c1ccc2cccc(-c3cccc(N(c4cccc(-c5ccccc5)c4)c4cccc(-c5ccccc5)c4-c4ccccc4)c3)c2c1]

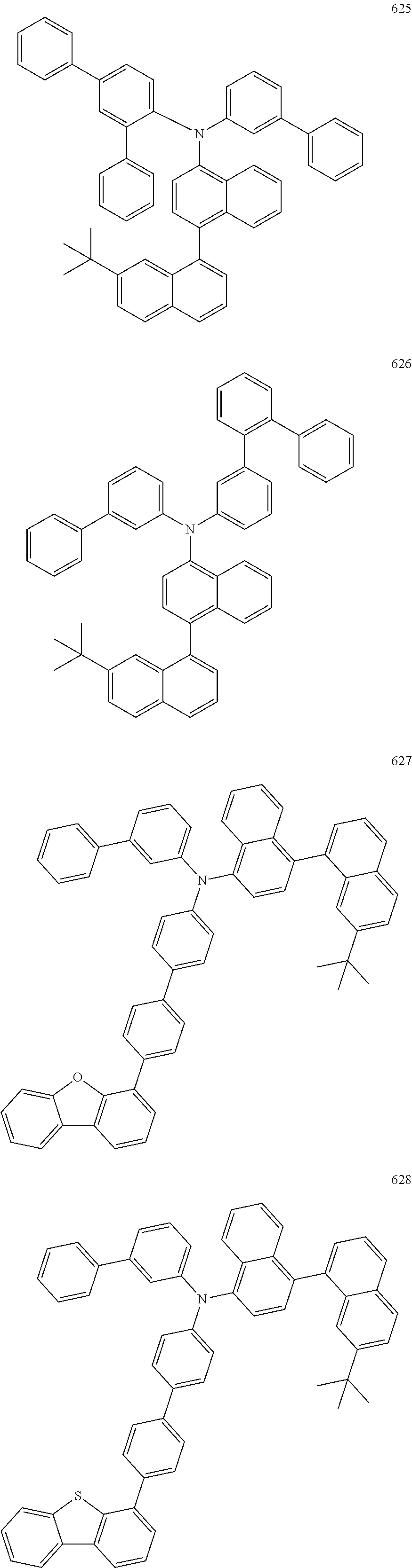 [SMILES: CC(C)(C)c1ccc2cccc(-c3ccc(N(c4cccc(-c5ccccc5)c4)c4ccc(-c5ccccc5)cc4-c4ccccc4)c4ccccc34)c2c1 ; CC(C)(C)c1ccc2cccc(-c3ccc(N(c4cccc(-c5ccccc5)c4)c4cccc(-c5ccccc5-c5ccccc5)c4)c4ccccc34)c2c1 ; CC(C)(C)c1ccc2cccc(-c3ccc(N(c4ccc(-c5ccc(-c6cccc7c6oc6ccccc67)cc5)cc4)c4cccc(-c5ccccc5)c4)c4ccccc34)c2c1 ; CC(C)(C)c1ccc2cccc(-c3ccc(N(c4ccc(-c5ccc(-c6cccc7c6sc6ccccc67)cc5)cc4)c4cccc(-c5ccccc5)c4)c4ccccc34)c2c1]

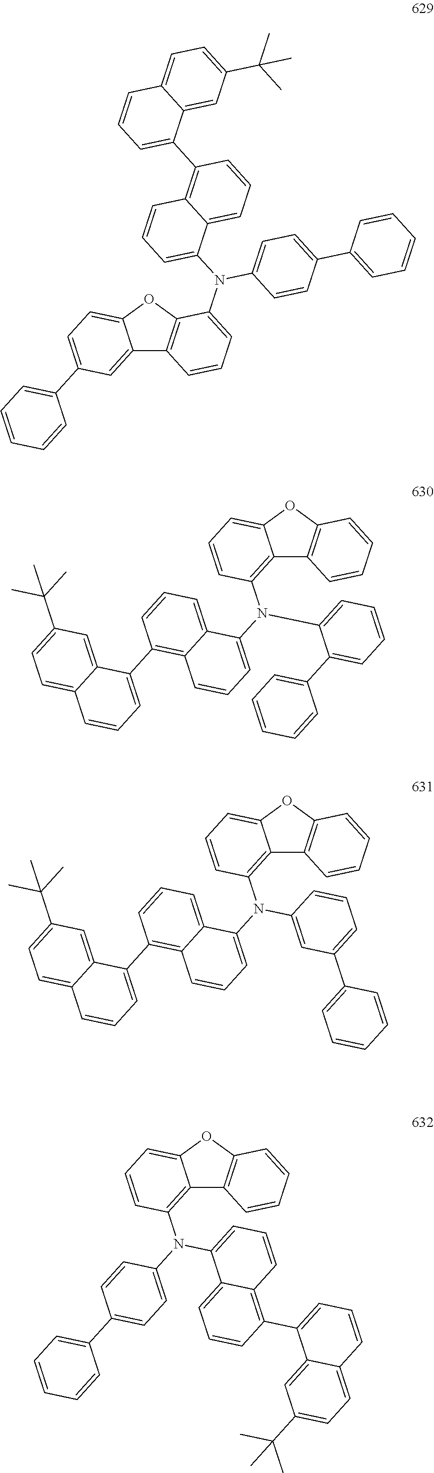 [SMILES: CC(C)(C)c1ccc2cccc(-c3cccc4c(N(c5ccc(-c6ccccc6)cc5)c5cccc6c5oc5ccc(-c7ccccc7)cc56)cccc34)c2c1 ; CC(C)(C)c1ccc2cccc(-c3cccc4c(N(c5ccccc5-c5ccccc5)c5cccc6oc7ccccc7c56)cccc34)c2c1 ; CC(C)(C)c1ccc2cccc(-c3cccc4c(N(c5cccc(-c6ccccc6)c5)c5cccc6oc7ccccc7c56)cccc34)c2c1 ; CC(C)(C)c1ccc2cccc(-c3cccc4c(N(c5ccc(-c6ccccc6)cc5)c5cccc6oc7ccccc7c56)cccc34)c2c1]

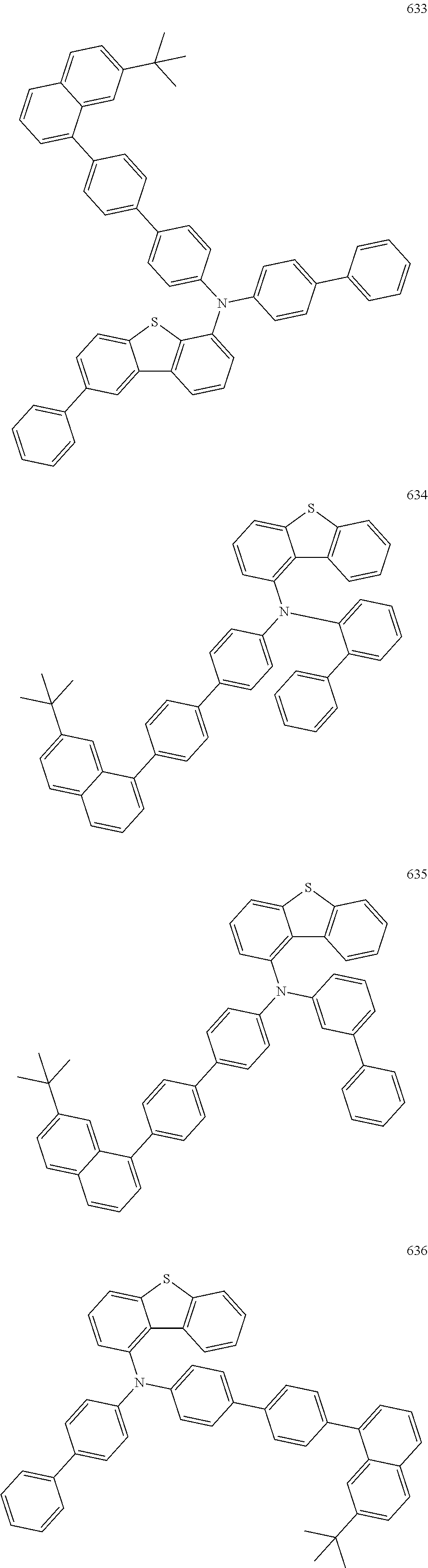 [SMILES: CC(C)(C)c1ccc2cccc(-c3ccc(-c4ccc(N(c5ccc(-c6ccccc6)cc5)c5cccc6c5sc5ccc(-c7ccccc7)cc56)cc4)cc3)c2c1 ; CC(C)(C)c1ccc2cccc(-c3ccc(-c4ccc(N(c5ccccc5-c5ccccc5)c5cccc6sc7ccccc7c56)cc4)cc3)c2c1 ; CC(C)(C)c1ccc2cccc(-c3ccc(-c4ccc(N(c5cccc(-c6ccccc6)c5)c5cccc6sc7ccccc7c56)cc4)cc3)c2c1 ; CC(C)(C)c1ccc2cccc(-c3ccc(-c4ccc(N(c5ccc(-c6ccccc6)cc5)c5cccc6sc7ccccc7c56)cc4)cc3)c2c1]

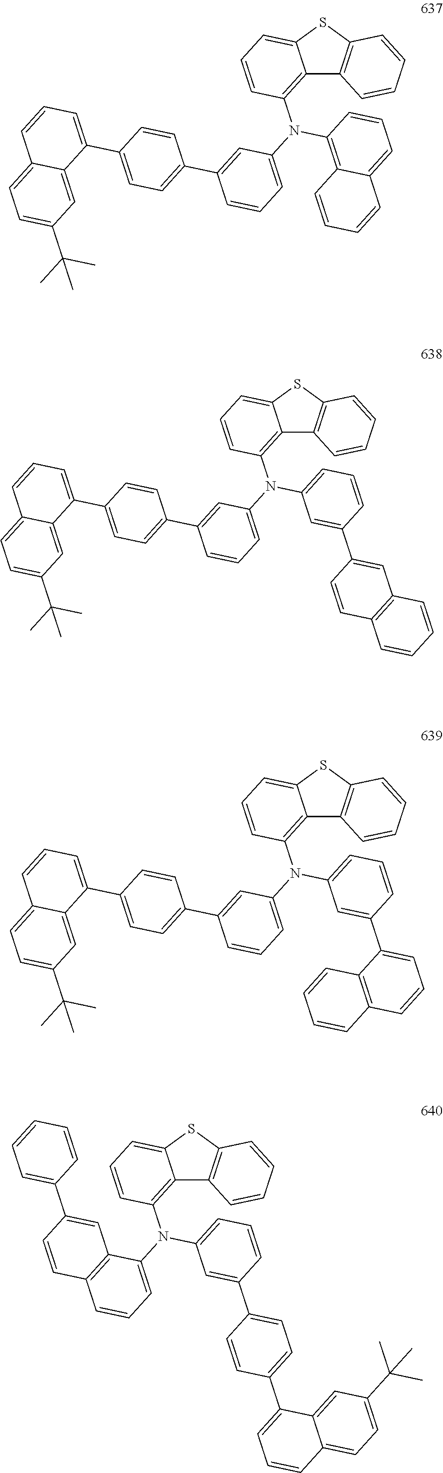 [SMILES: CC(C)(C)c1ccc2cccc(-c3ccc(-c4cccc(N(c5cccc6ccccc56)c5cccc6sc7ccccc7c56)c4)cc3)c2c1 ; CC(C)(C)c1ccc2cccc(-c3ccc(-c4cccc(N(c5cccc(-c6ccc7ccccc7c6)c5)c5cccc6sc7ccccc7c56)c4)cc3)c2c1 ; CC(C)(C)c1ccc2cccc(-c3ccc(-c4cccc(N(c5cccc(-c6cccc7ccccc67)c5)c5cccc6sc7ccccc7c56)c4)cc3)c2c1 ; CC(C)(C)c1ccc2cccc(-c3ccc(-c4cccc(N(c5cccc6ccc(-c7ccccc7)cc56)c5cccc6sc7ccccc7c56)c4)cc3)c2c1]

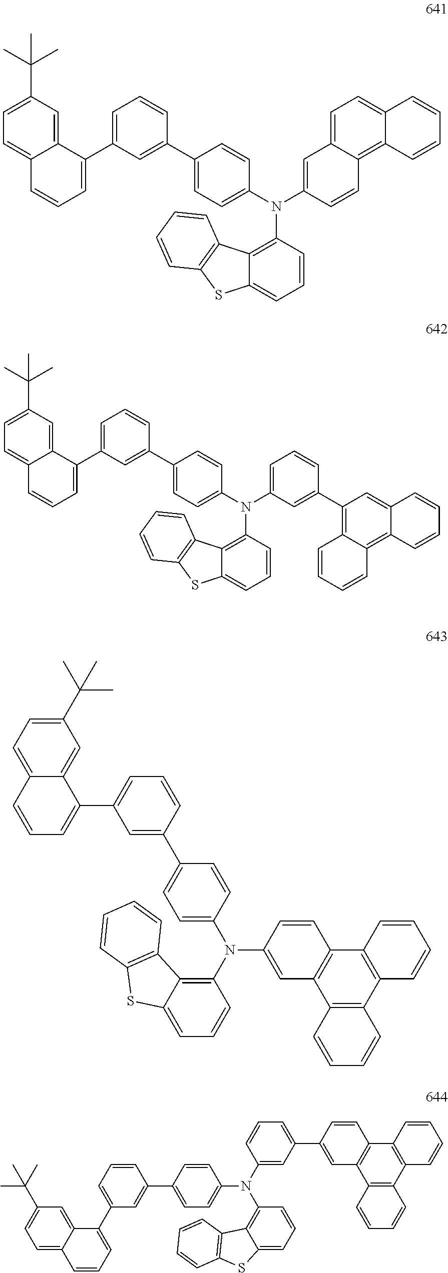 [SMILES: CC(C)(C)c1ccc2cccc(-c3cccc(-c4ccc(N(c5ccc6c(ccc7ccccc76)c5)c5cccc6sc7ccccc7c56)cc4)c3)c2c1 ; CC(C)(C)c1ccc2cccc(-c3cccc(-c4ccc(N(c5cccc(-c6cc7ccccc7c7ccccc67)c5)c5cccc6sc7ccccc7c56)cc4)c3)c2c1 ; CC(C)(C)c1ccc2cccc(-c3cccc(-c4ccc(N(c5ccc6c7ccccc7c7ccccc7c6c5)c5cccc6sc7ccccc7c56)cc4)c3)c2c1 ; CC(C)(C)c1ccc2cccc(-c3cccc(-c4ccc(N(c5cccc(-c6ccc7c8ccccc8c8ccccc8c7c6)c5)c5cccc6sc7ccccc7c56)cc4)c3)c2c1]

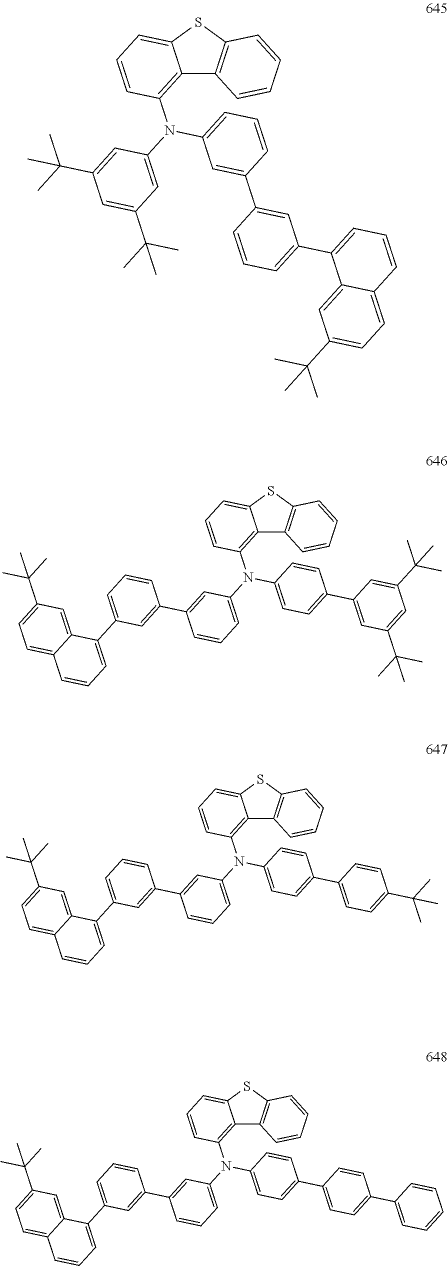

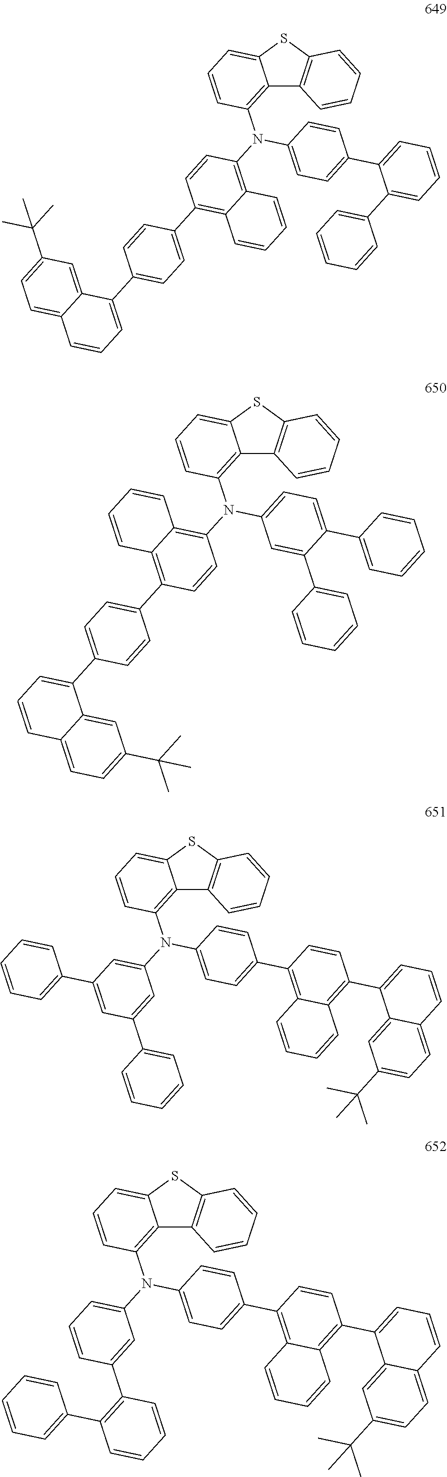 [SMILES: CC(C)(C)c1ccc2cccc(-c3ccc(-c4ccc(N(c5ccc(-c6ccccc6-c6ccccc6)cc5)c5cccc6sc7ccccc7c56)c5ccccc45)cc3)c2c1 ; CC(C)(C)c1ccc2cccc(-c3ccc(-c4ccc(N(c5ccc(-c6ccccc6)c(-c6ccccc6)c5)c5cccc6sc7ccccc7c56)c5ccccc45)cc3)c2c1 ; CC(C)(C)c1ccc2cccc(-c3ccc(-c4ccc(N(c5cc(-c6ccccc6)cc(-c6ccccc6)c5)c5cccc6sc7ccccc7c56)cc4)c4ccccc34)c2c1 ; CC(C)(C)c1ccc2cccc(-c3ccc(-c4ccc(N(c5cccc(-c6ccccc6-c6ccccc6)c5)c5cccc6sc7ccccc7c56)cc4)c4ccccc34)c2c1]

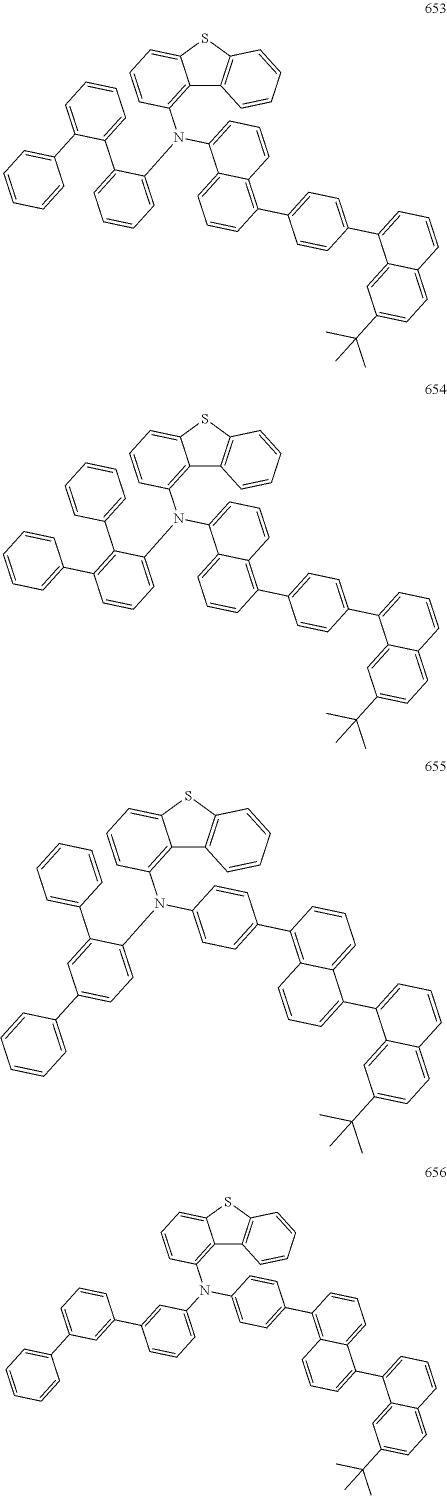

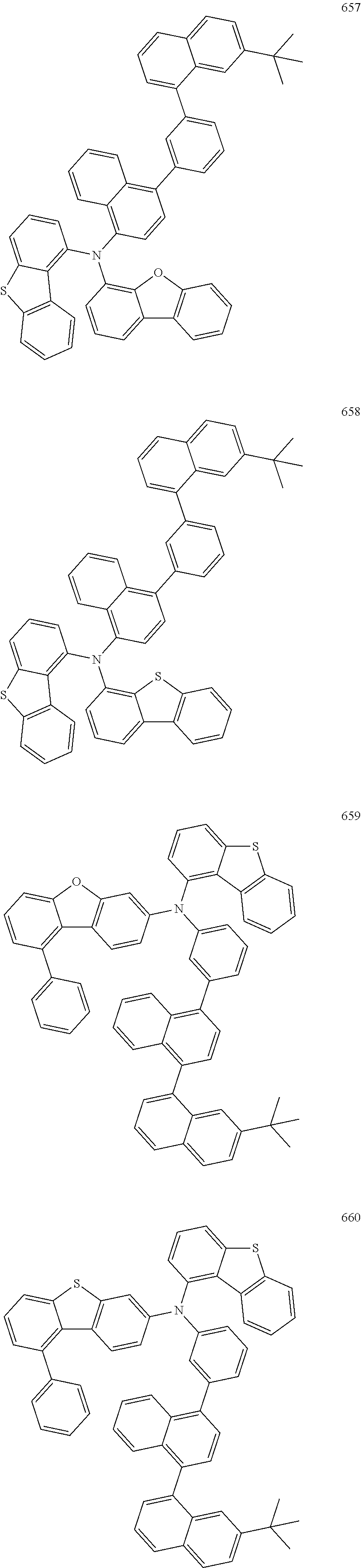 [SMILES: CC(C)(C)c1ccc2cccc(-c3cccc(-c4ccc(N(c5cccc6c5oc5ccccc56)c5cccc6sc7ccccc7c56)c5ccccc45)c3)c2c1 ; CC(C)(C)c1ccc2cccc(-c3cccc(-c4ccc(N(c5cccc6c5sc5ccccc56)c5cccc6sc7ccccc7c56)c5ccccc45)c3)c2c1 ; CC(C)(C)c1ccc2cccc(-c3ccc(-c4cccc(N(c5ccc6c(c5)oc5cccc(-c7ccccc7)c56)c5cccc6sc7ccccc7c56)c4)c4ccccc34)c2c1 ; CC(C)(C)c1ccc2cccc(-c3ccc(-c4cccc(N(c5ccc6c(c5)sc5cccc(-c7ccccc7)c56)c5cccc6sc7ccccc7c56)c4)c4ccccc34)c2c1]

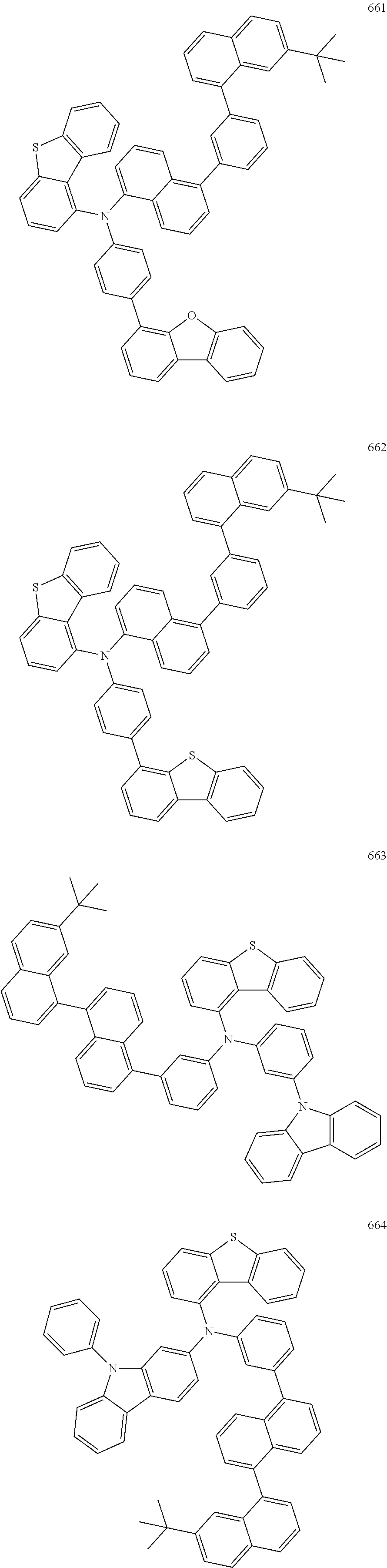 [SMILES: CC(C)(C)c1ccc2cccc(-c3cccc(-c4cccc5c(N(c6ccc(-c7cccc8c7oc7ccccc78)cc6)c6cccc7sc8ccccc8c67)cccc45)c3)c2c1 ; CC(C)(C)c1ccc2cccc(-c3cccc(-c4cccc5c(N(c6ccc(-c7cccc8c7sc7ccccc78)cc6)c6cccc7sc8ccccc8c67)cccc45)c3)c2c1 ; CC(C)(C)c1ccc2cccc(-c3cccc4c(-c5cccc(N(c6cccc(-n7c8ccccc8c8ccccc87)c6)c6cccc7sc8ccccc8c67)c5)cccc34)c2c1 ; CC(C)(C)c1ccc2cccc(-c3cccc4c(-c5cccc(N(c6ccc7c8ccccc8n(-c8ccccc8)c7c6)c6cccc7sc8ccccc8c67)c5)cccc34)c2c1]

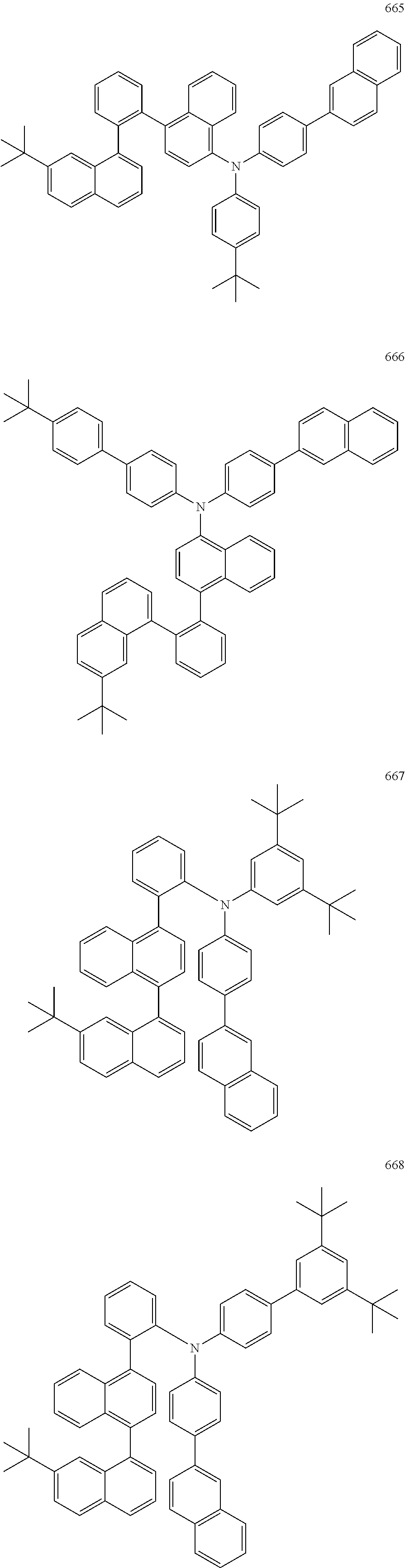

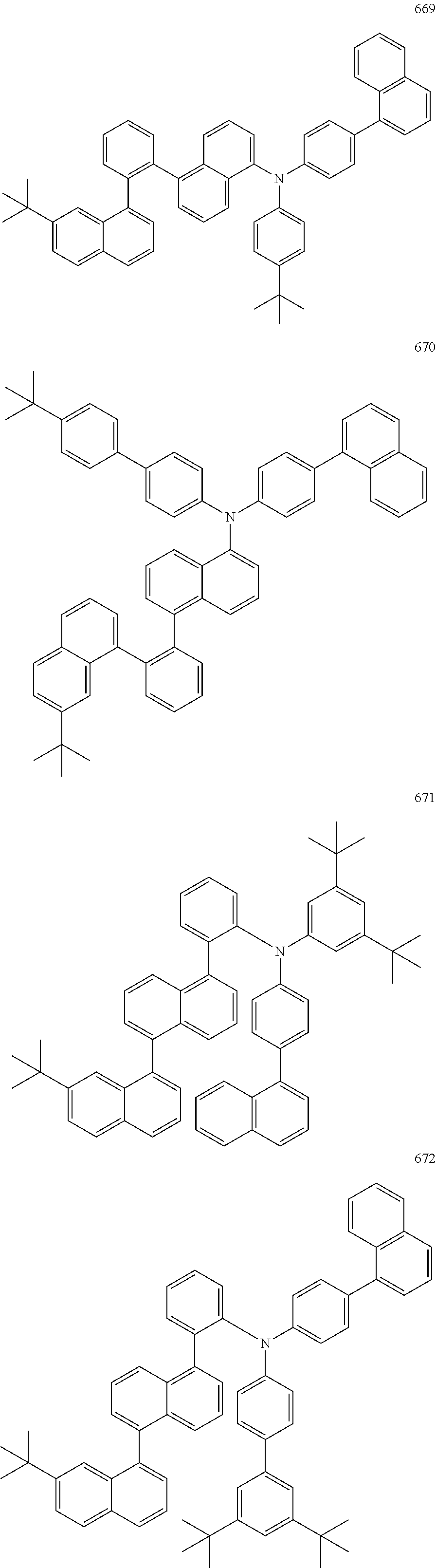

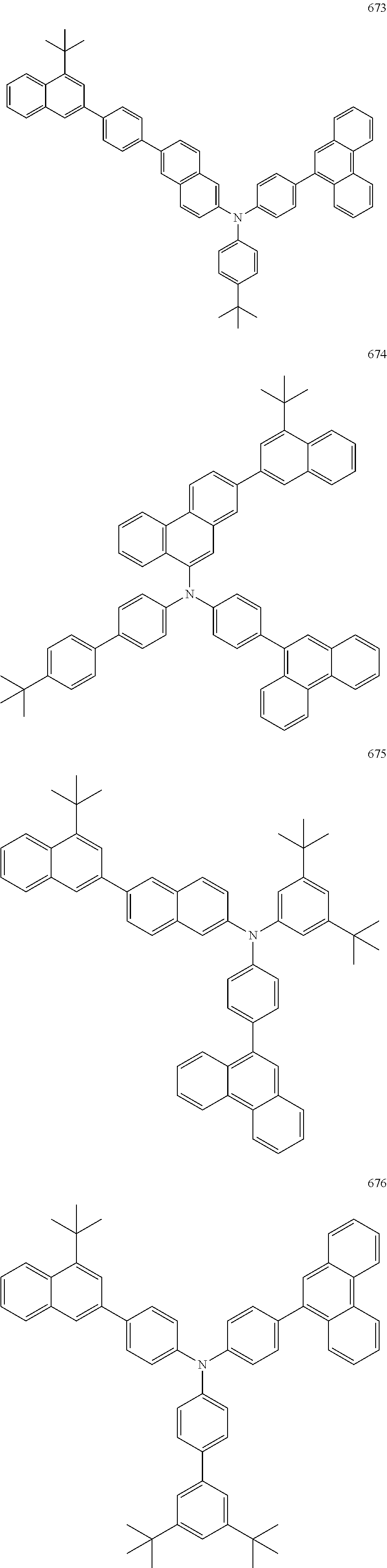 [SMILES: CC(C)(C)c1ccc(N(c2ccc(-c3cc4ccccc4c4ccccc34)cc2)c2ccc3cc(-c4ccc(-c5cc(C(C)(C)C)c6ccccc6c5)cc4)ccc3c2)cc1 ; CC(C)(C)c1ccc(-c2ccc(N(c3ccc(-c4cc5ccccc5c5ccccc45)cc3)c3cc4cc(-c5cc(C(C)(C)C)c6ccccc6c5)ccc4c4ccccc34)cc2)cc1 ; CC(C)(C)c1cc(N(c2ccc(-c3cc4ccccc4c4ccccc34)cc2)c2ccc3cc(-c4cc(C(C)(C)C)c5ccccc5c4)ccc3c2)cc(C(C)(C)C)c1 ; CC(C)(C)c1cc(-c2ccc(N(c3ccc(-c4cc(C(C)(C)C)c5ccccc5c4)cc3)c3ccc(-c4cc5ccccc5c5ccccc45)cc3)cc2)cc(C(C)(C)C)c1]

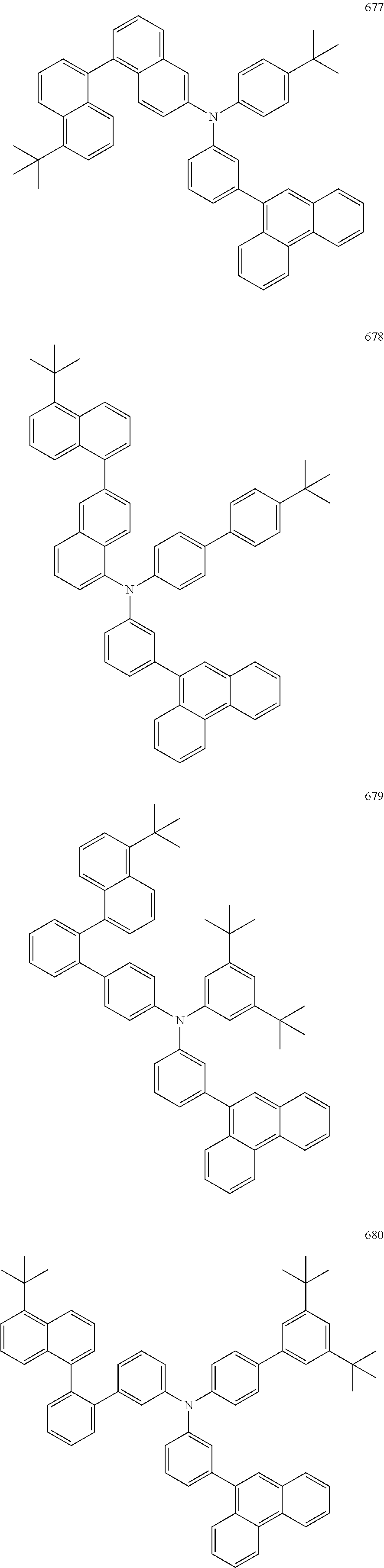 [SMILES: CC(C)(C)c1ccc(N(c2cccc(-c3cc4ccccc4c4ccccc34)c2)c2ccc3c(-c4cccc5c(C(C)(C)C)cccc45)cccc3c2)cc1 ; CC(C)(C)c1ccc(-c2ccc(N(c3cccc(-c4cc5ccccc5c5ccccc45)c3)c3cccc4cc(-c5cccc6c(C(C)(C)C)cccc56)ccc34)cc2)cc1 ; CC(C)(C)c1cc(N(c2ccc(-c3ccccc3-c3cccc4c(C(C)(C)C)cccc34)cc2)c2cccc(-c3cc4ccccc4c4ccccc34)c2)cc(C(C)(C)C)c1 ; CC(C)(C)c1cc(-c2ccc(N(c3cccc(-c4ccccc4-c4cccc5c(C(C)(C)C)cccc45)c3)c3cccc(-c4cc5ccccc5c5ccccc45)c3)cc2)cc(C(C)(C)C)c1]

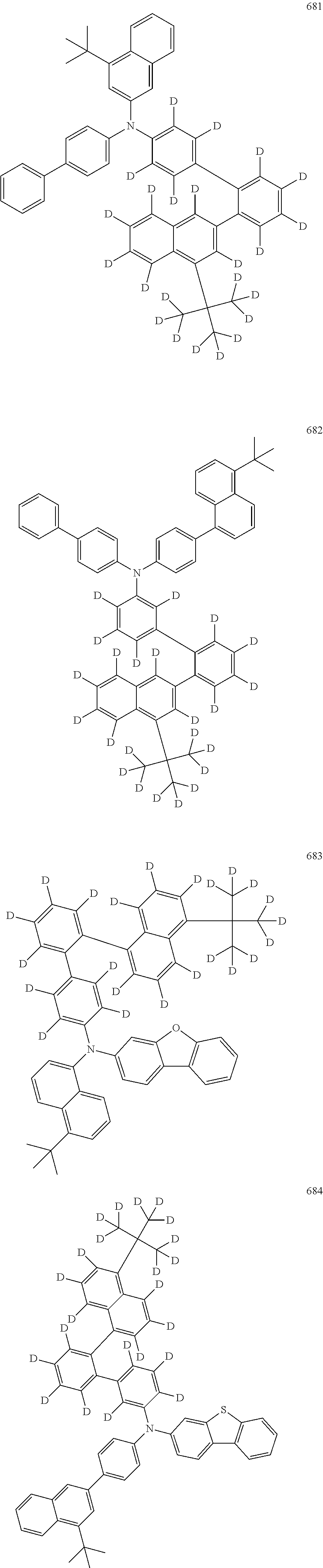

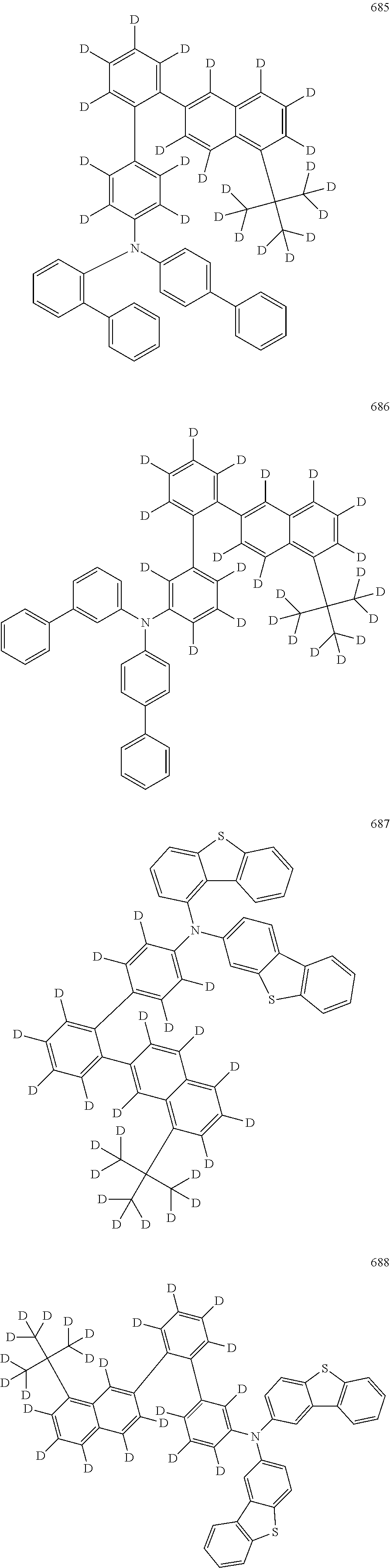

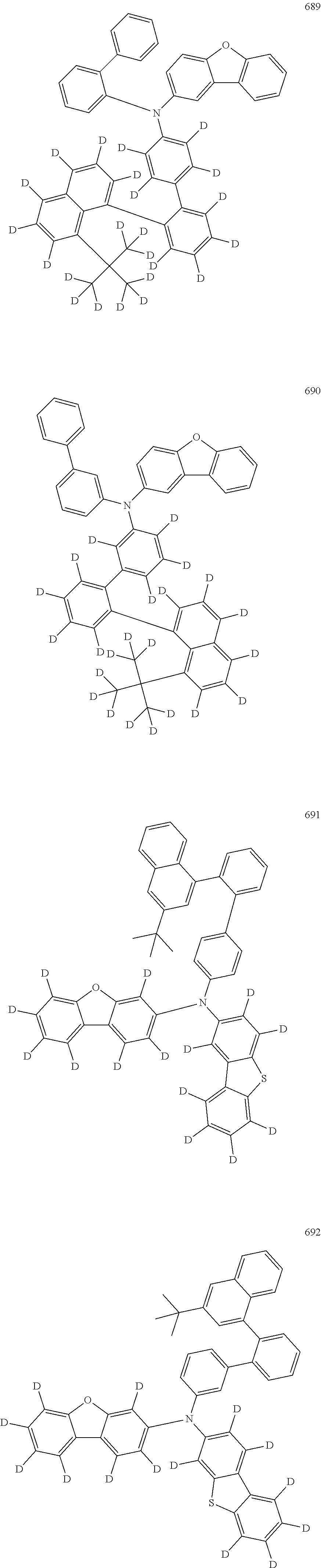

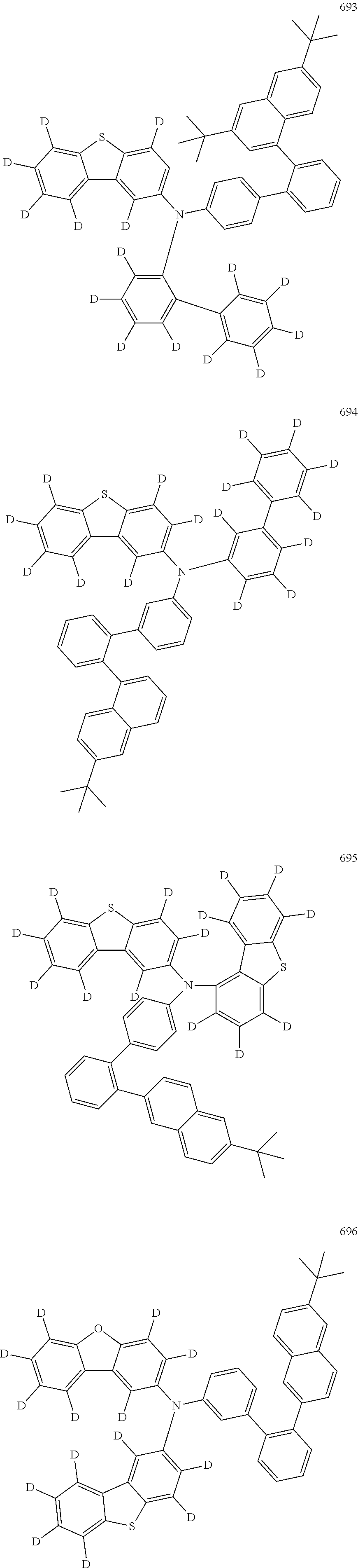

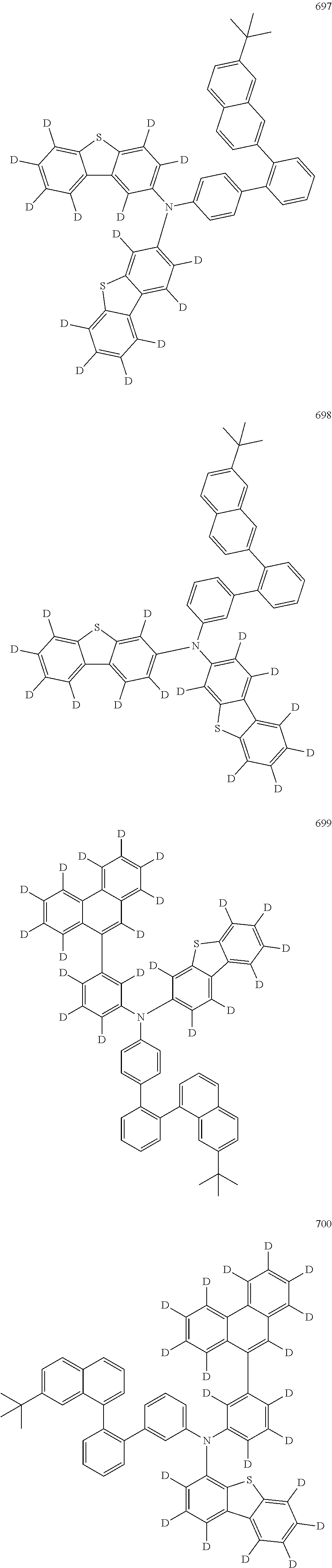

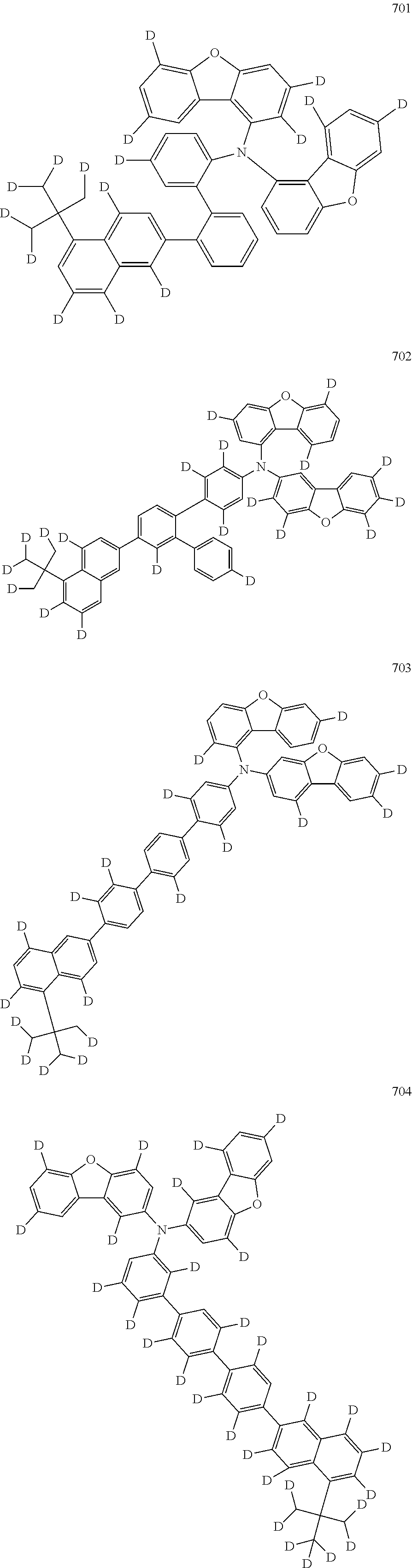

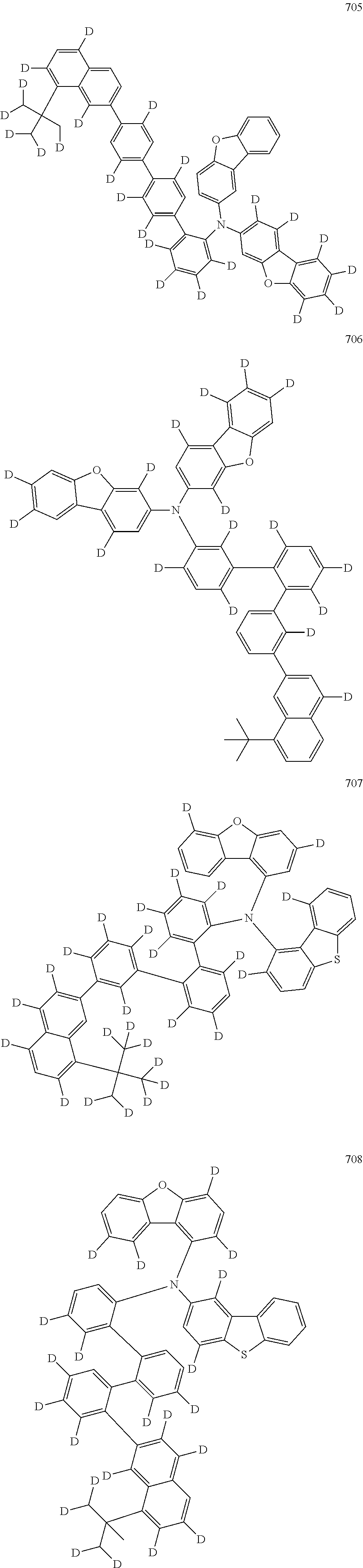

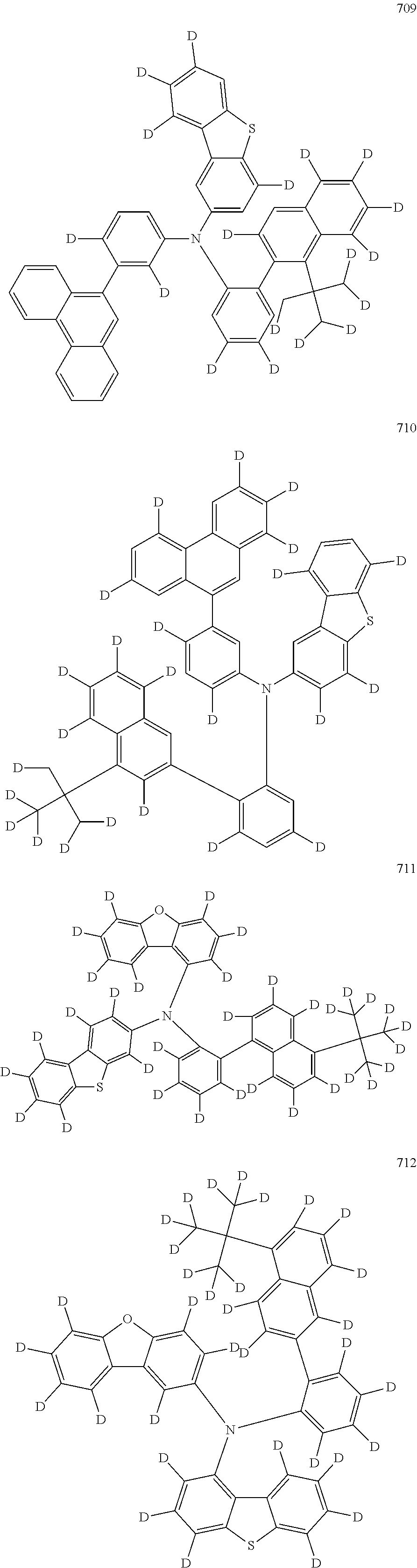

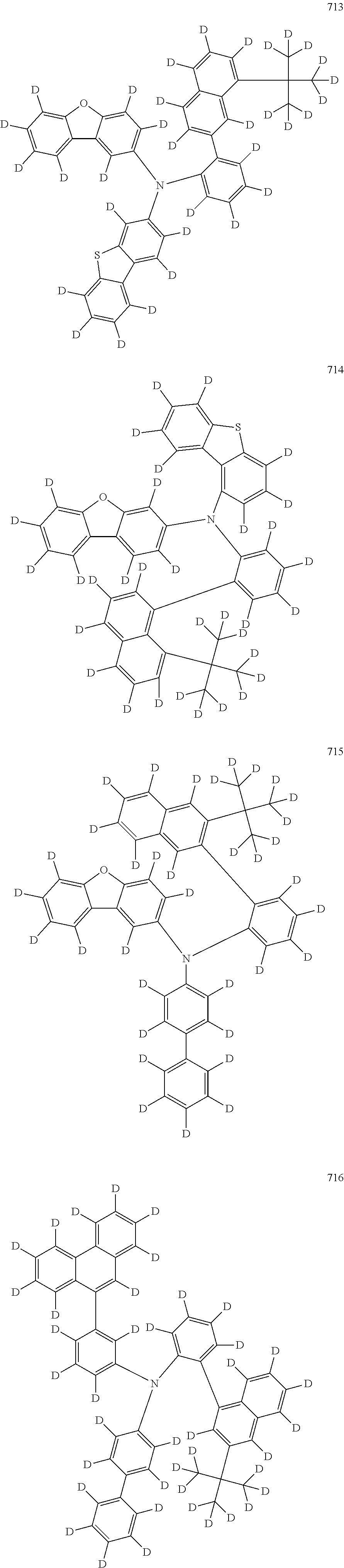

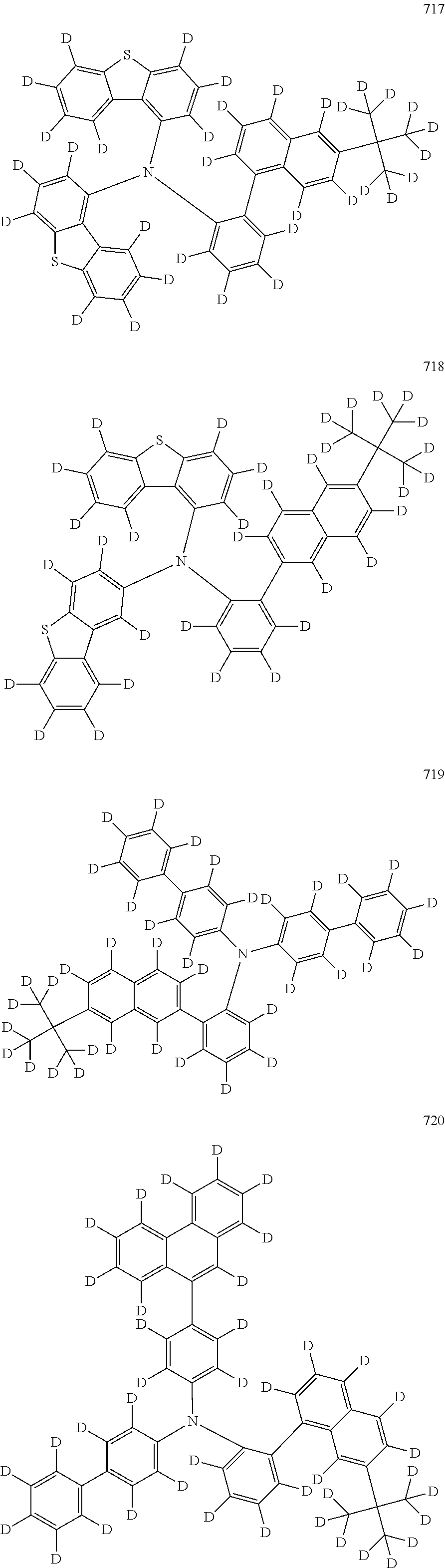

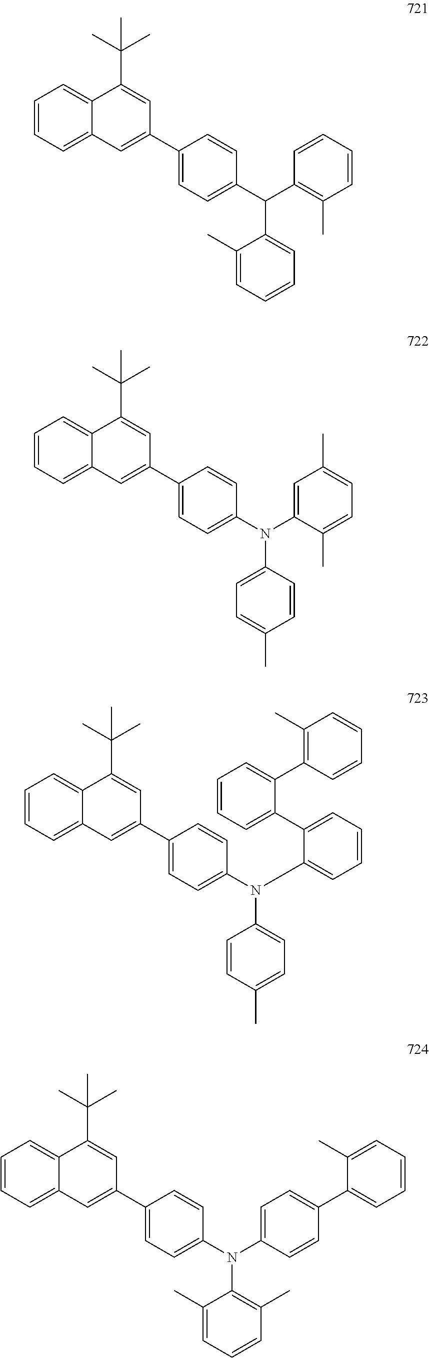

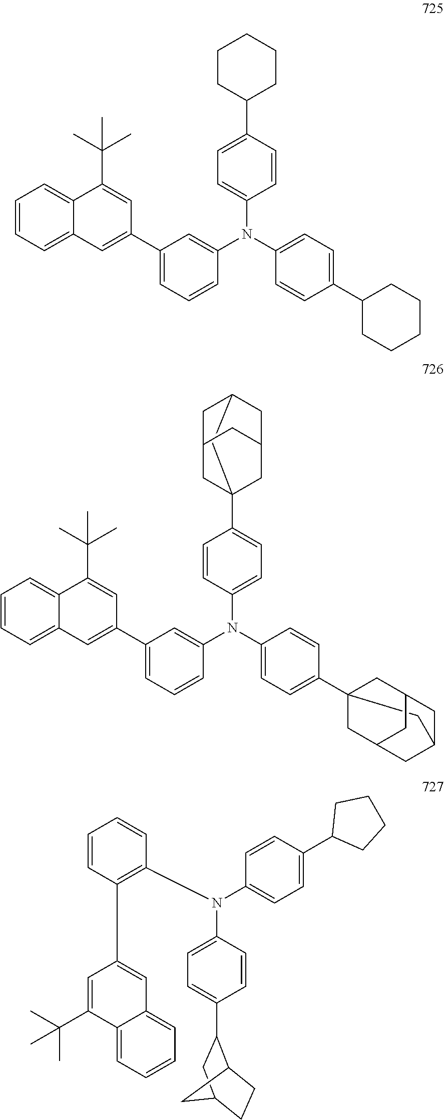 [SMILES: CC(C)(C)c1cc(-c2cccc(N(c3ccc(C4CCCCC4)cc3)c3ccc(C4CCCCC4)cc3)c2)cc2ccccc12 ; CC(C)(C)c1cc(-c2cccc(N(c3ccc(C45CC6CC(CC(C6)C4)C5)cc3)c3ccc(C45CC6CC(CC(C6)C4)C5)cc3)c2)cc2ccccc12 ; CC(C)(C)c1cc(-c2ccccc2N(c2ccc(C3CCCC3)cc2)c2ccc(C3CC4CCC3C4)cc2)cc2ccccc12]

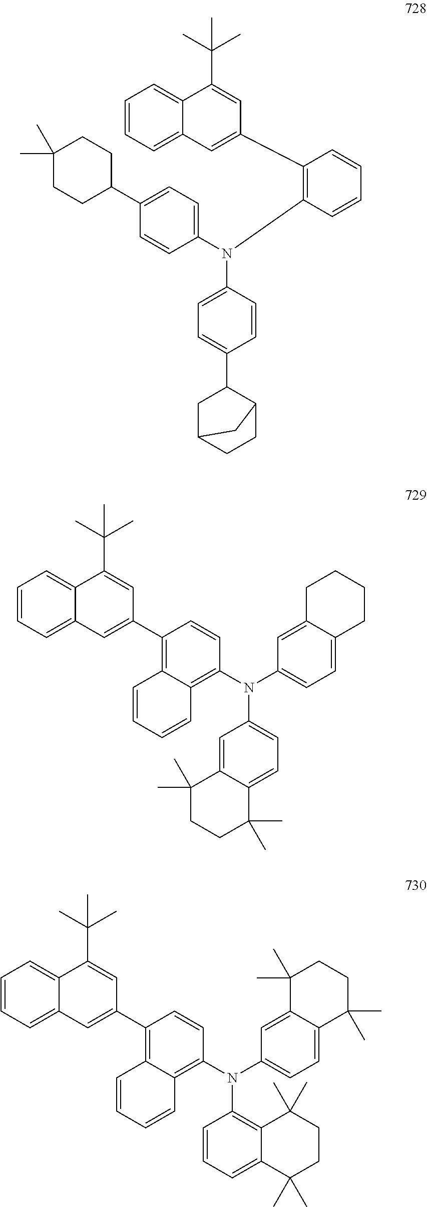 [SMILES: CC1(C)CCC(c2ccc(N(c3ccc(C4CC5CCC4C5)cc3)c3ccccc3-c3cc(C(C)(C)C)c4ccccc4c3)cc2)CC1 ; CC(C)(C)c1cc(-c2ccc(N(c3ccc4c(c3)CCCC4)c3ccc4c(c3)C(C)(C)CCC4(C)C)c3ccccc23)cc2ccccc12 ; CC(C)(C)c1cc(-c2ccc(N(c3ccc4c(c3)C(C)(C)CCC4(C)C)c3cccc4c3C(C)(C)CCC4(C)C)c3ccccc23)cc2ccccc12]

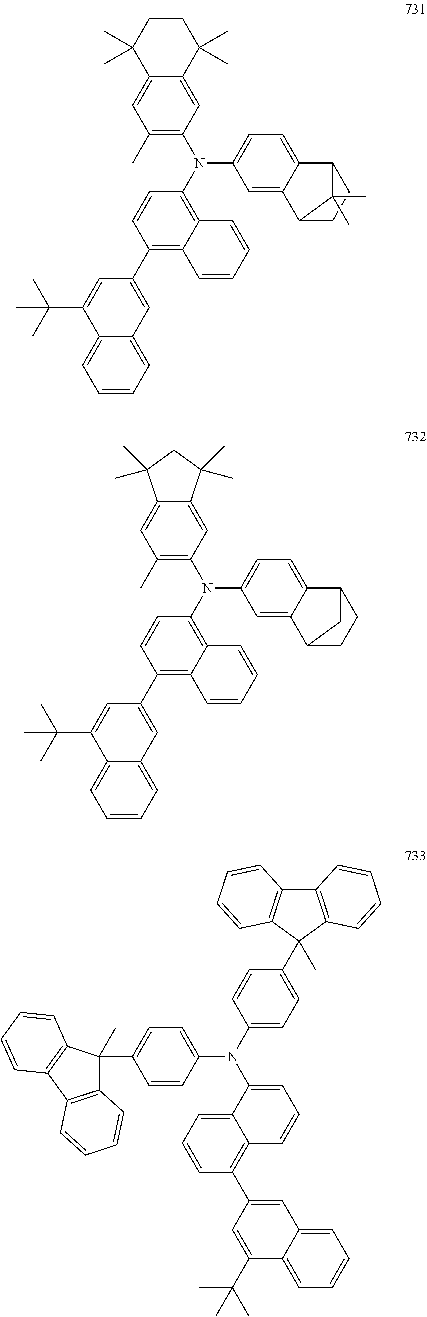

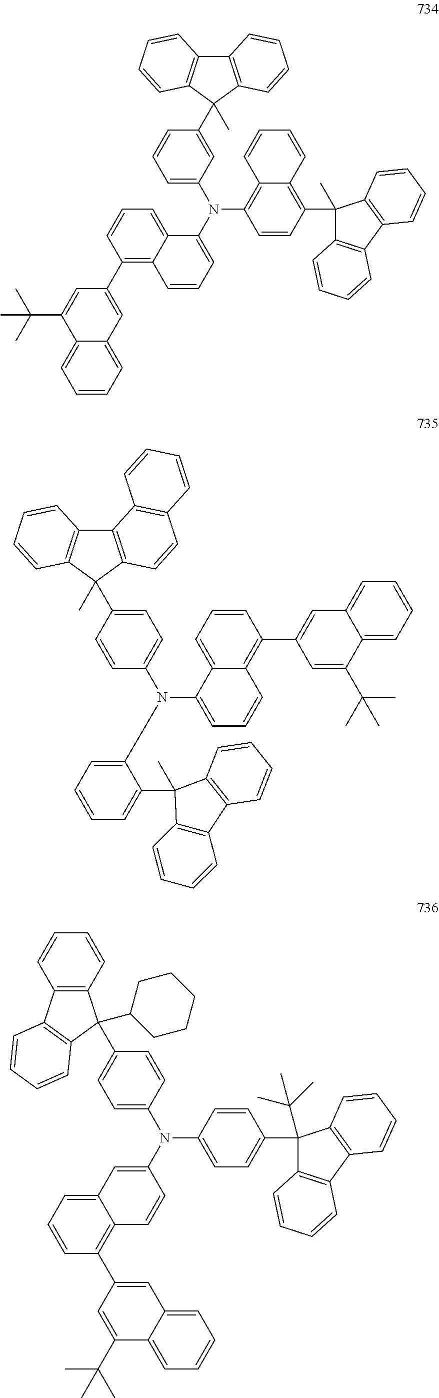 [SMILES: CC(C)(C)c1cc(-c2cccc3c(N(c4cccc(C5(C)c6ccccc6-c6ccccc65)c4)c4ccc(C5(C)c6ccccc6-c6ccccc65)c5ccccc45)cccc23)cc2ccccc12 ; CC(C)(C)c1cc(-c2cccc3c(N(c4ccc(C5(C)c6ccccc6-c6c5ccc5ccccc65)cc4)c4ccccc4C4(C)c5ccccc5-c5ccccc54)cccc23)cc2ccccc12 ; CC(C)(C)c1cc(-c2cccc3cc(N(c4ccc(C5(C6CCCCC6)c6ccccc6-c6ccccc65)cc4)c4ccc(C5(C(C)(C)C)c6ccccc6-c6ccccc65)cc4)ccc23)cc2ccccc12]

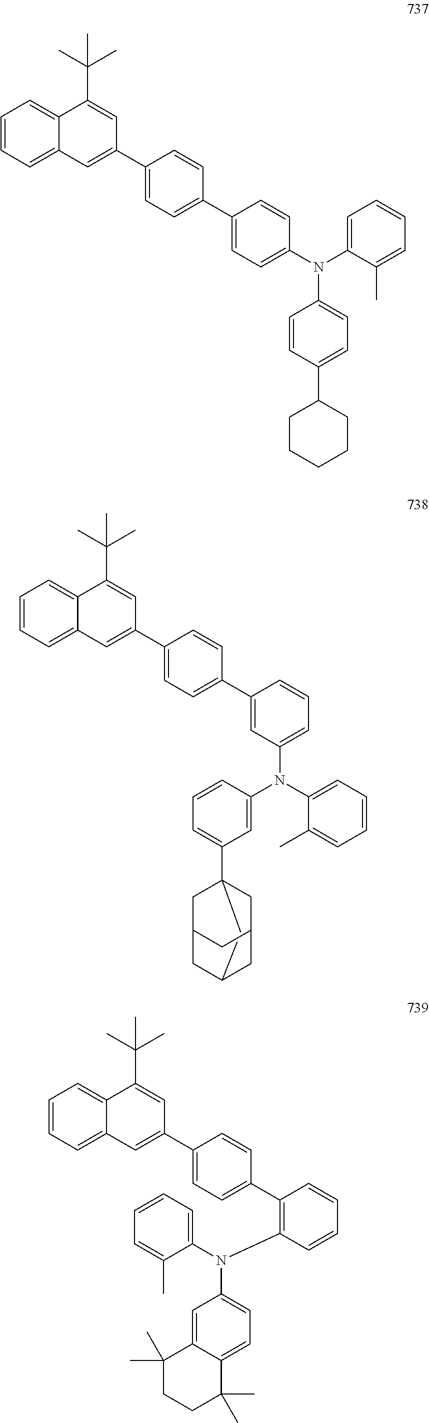 [SMILES: Cc1ccccc1N(c1ccc(-c2ccc(-c3cc(C(C)(C)C)c4ccccc4c3)cc2)cc1)c1ccc(C2CCCCC2)cc1 ; Cc1ccccc1N(c1cccc(-c2ccc(-c3cc(C(C)(C)C)c4ccccc4c3)cc2)c1)c1cccc(C23CC4CC(CC(C4)C2)C3)c1 ; Cc1ccccc1N(c1ccc2c(c1)C(C)(C)CCC2(C)C)c1ccccc1-c1ccc(-c2cc(C(C)(C)C)c3ccccc3c2)cc1]

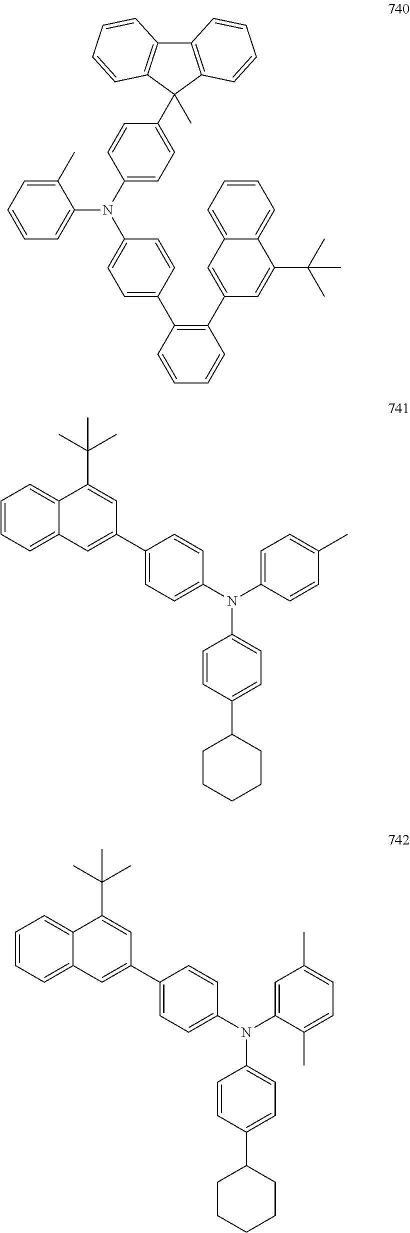 [SMILES: Cc1ccccc1N(c1ccc(-c2ccccc2-c2cc(C(C)(C)C)c3ccccc3c2)cc1)c1ccc(C2(C)c3ccccc3-c3ccccc32)cc1 ; Cc1ccc(N(c2ccc(-c3cc(C(C)(C)C)c4ccccc4c3)cc2)c2ccc(C3CCCCC3)cc2)cc1 ; Cc1ccc(C)c(N(c2ccc(-c3cc(C(C)(C)C)c4ccccc4c3)cc2)c2ccc(C3CCCCC3)cc2)c1]

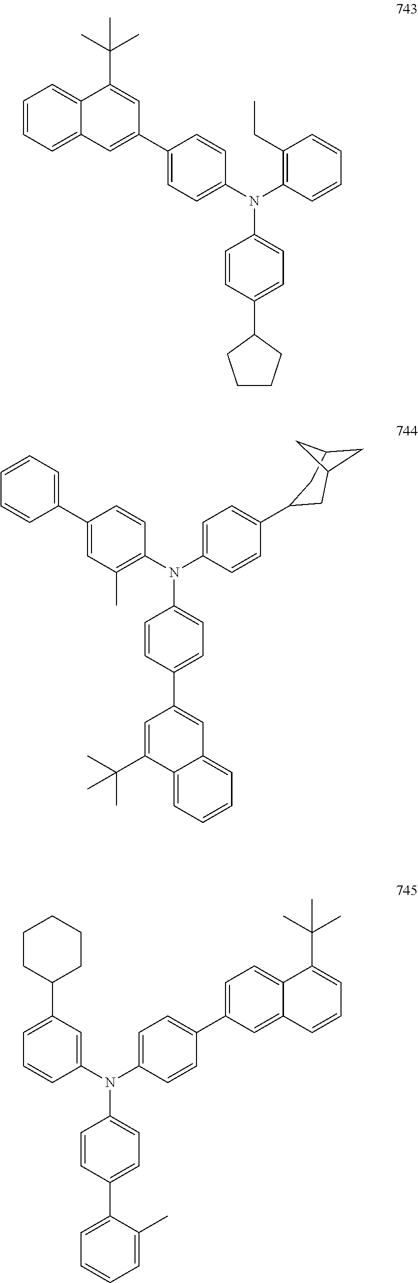 [SMILES: CCc1ccccc1N(c1ccc(-c2cc(C(C)(C)C)c3ccccc3c2)cc1)c1ccc(C2CCCC2)cc1 ; Cc1cc(-c2ccccc2)ccc1N(c1ccc(-c2cc(C(C)(C)C)c3ccccc3c2)cc1)c1ccc(C2CC3CC(C3)C2)cc1 ; Cc1ccccc1-c1ccc(N(c2ccc(-c3ccc4c(C(C)(C)C)cccc4c3)cc2)c2cccc(C3CCCCC3)c2)cc1]

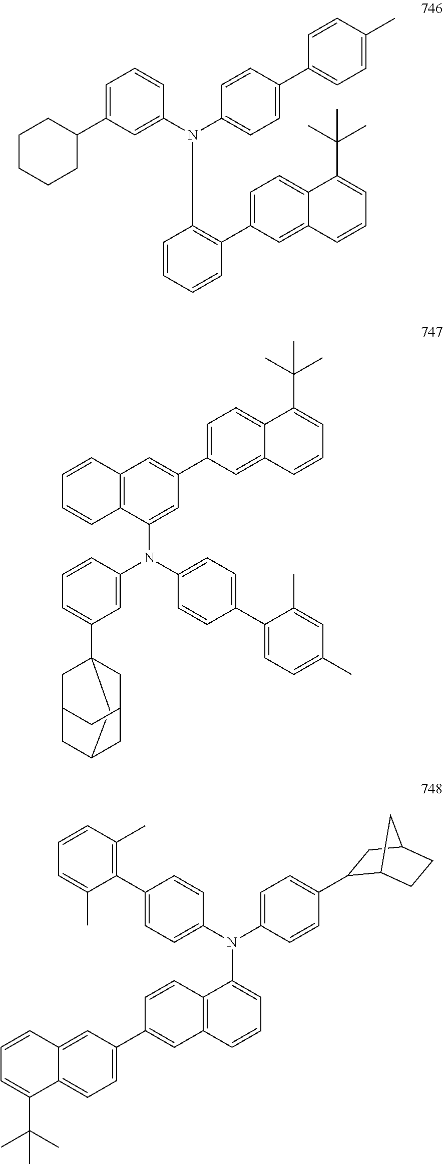 [SMILES: Cc1ccc(-c2ccc(N(c3cccc(C4CCCCC4)c3)c3ccccc3-c3ccc4c(C(C)(C)C)cccc4c3)cc2)cc1 ; Cc1ccc(-c2ccc(N(c3cccc(C45CC6CC(CC(C6)C4)C5)c3)c3cc(-c4ccc5c(C(C)(C)C)cccc5c4)cc4ccccc34)cc2)c(C)c1 ; Cc1cccc(C)c1-c1ccc(N(c2ccc(C3CC4CCC3C4)cc2)c2cccc3cc(-c4ccc5c(C(C)(C)C)cccc5c4)ccc23)cc1]

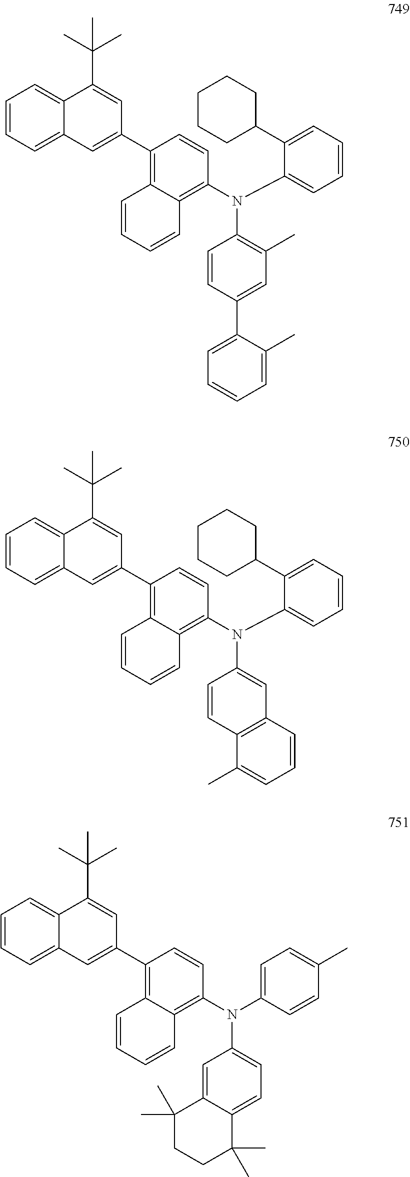 [SMILES: Cc1ccccc1-c1ccc(N(c2ccccc2C2CCCCC2)c2ccc(-c3cc(C(C)(C)C)c4ccccc4c3)c3ccccc23)c(C)c1 ; Cc1cccc2cc(N(c3ccccc3C3CCCCC3)c3ccc(-c4cc(C(C)(C)C)c5ccccc5c4)c4ccccc34)ccc12 ; Cc1ccc(N(c2ccc3c(c2)C(C)(C)CCC3(C)C)c2ccc(-c3cc(C(C)(C)C)c4ccccc4c3)c3ccccc23)cc1]

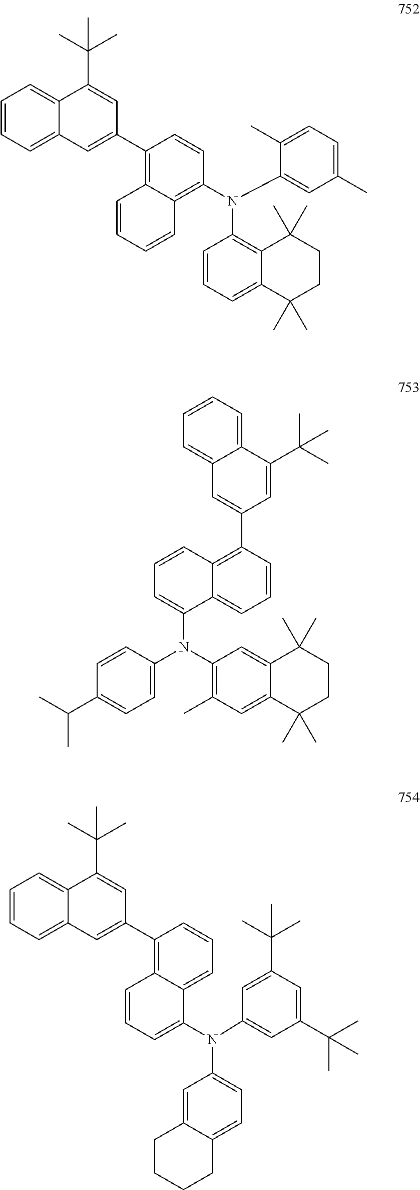

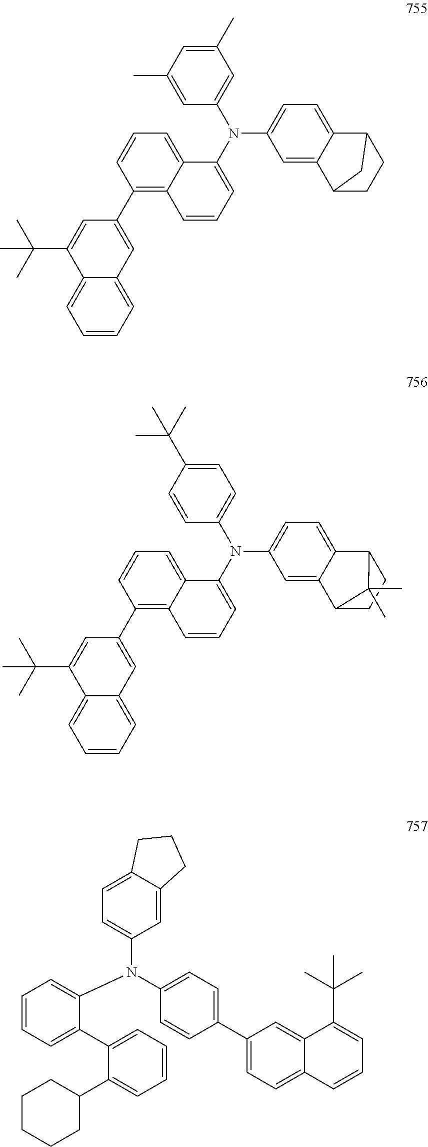 [SMILES: Cc1cc(C)cc(N(c2ccc3c(c2)C2CCC3C2)c2cccc3c(-c4cc(C(C)(C)C)c5ccccc5c4)cccc23)c1 ; CC(C)(C)c1ccc(N(c2ccc3c(c2)C2CCC3C2(C)C)c2cccc3c(-c4cc(C(C)(C)C)c5ccccc5c4)cccc23)cc1 ; CC(C)(C)c1cccc2ccc(-c3ccc(N(c4ccc5c(c4)CCC5)c4ccccc4-c4ccccc4C4CCCCC4)cc3)cc12]

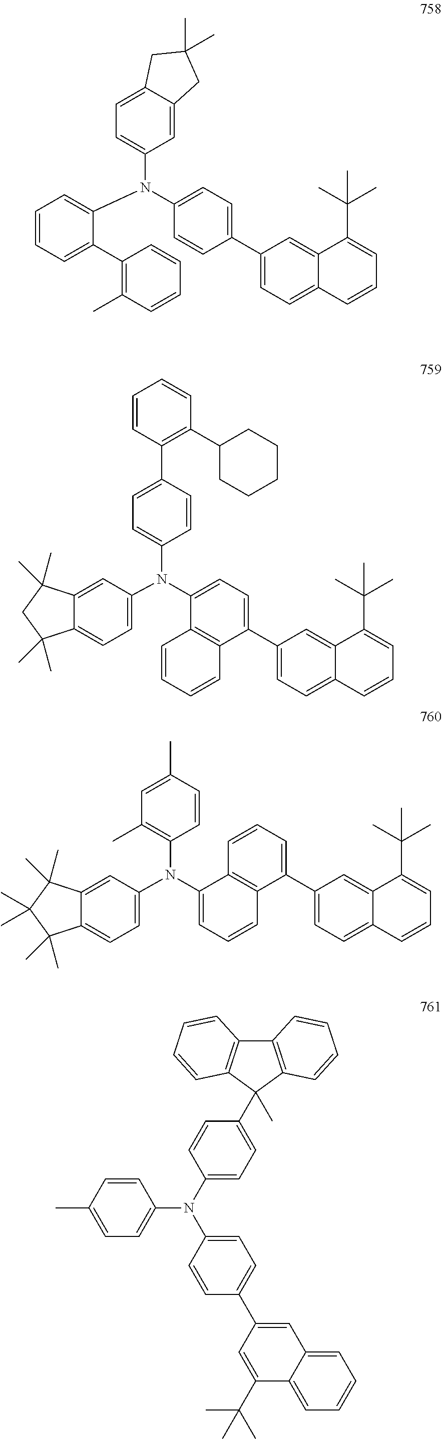

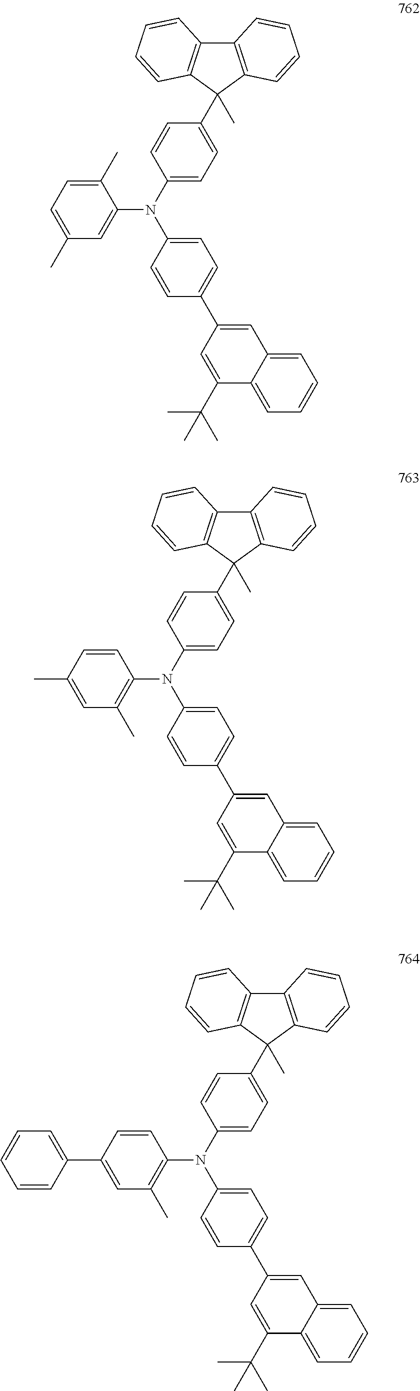 [SMILES: Cc1ccc(C)c(N(c2ccc(-c3cc(C(C)(C)C)c4ccccc4c3)cc2)c2ccc(C3(C)c4ccccc4-c4ccccc43)cc2)c1 ; Cc1ccc(N(c2ccc(-c3cc(C(C)(C)C)c4ccccc4c3)cc2)c2ccc(C3(C)c4ccccc4-c4ccccc43)cc2)c(C)c1 ; Cc1cc(-c2ccccc2)ccc1N(c1ccc(-c2cc(C(C)(C)C)c3ccccc3c2)cc1)c1ccc(C2(C)c3ccccc3-c3ccccc32)cc1]

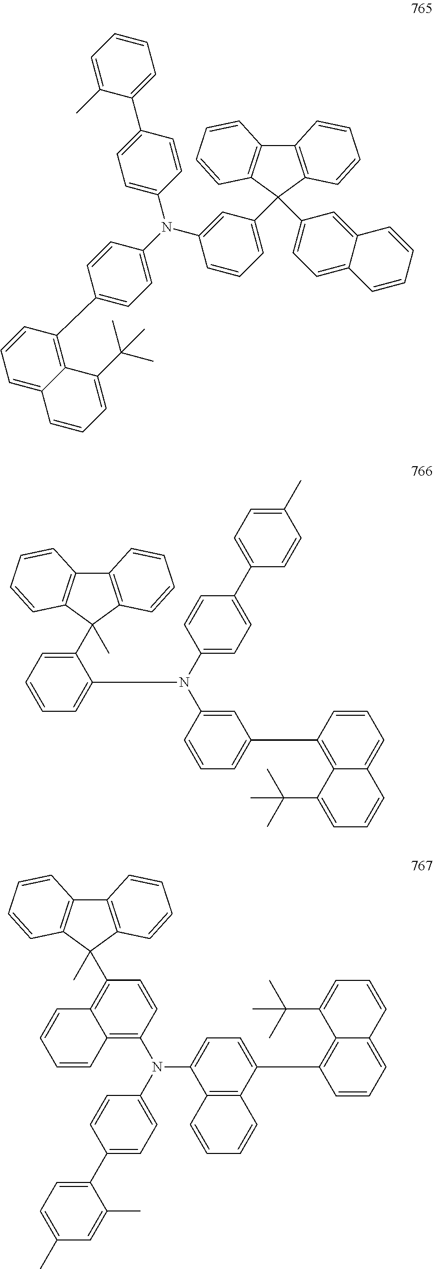 [SMILES: Cc1ccccc1-c1ccc(N(c2ccc(-c3cccc4cccc(C(C)(C)C)c34)cc2)c2cccc(C3(c4ccc5ccccc5c4)c4ccccc4-c4ccccc43)c2)cc1 ; Cc1ccc(-c2ccc(N(c3cccc(-c4cccc5cccc(C(C)(C)C)c45)c3)c3ccccc3C3(C)c4ccccc4-c4ccccc43)cc2)cc1 ; Cc1ccc(-c2ccc(N(c3ccc(-c4cccc5cccc(C(C)(C)C)c45)c4ccccc34)c3ccc(C4(C)c5ccccc5-c5ccccc54)c4ccccc34)cc2)c(C)c1]

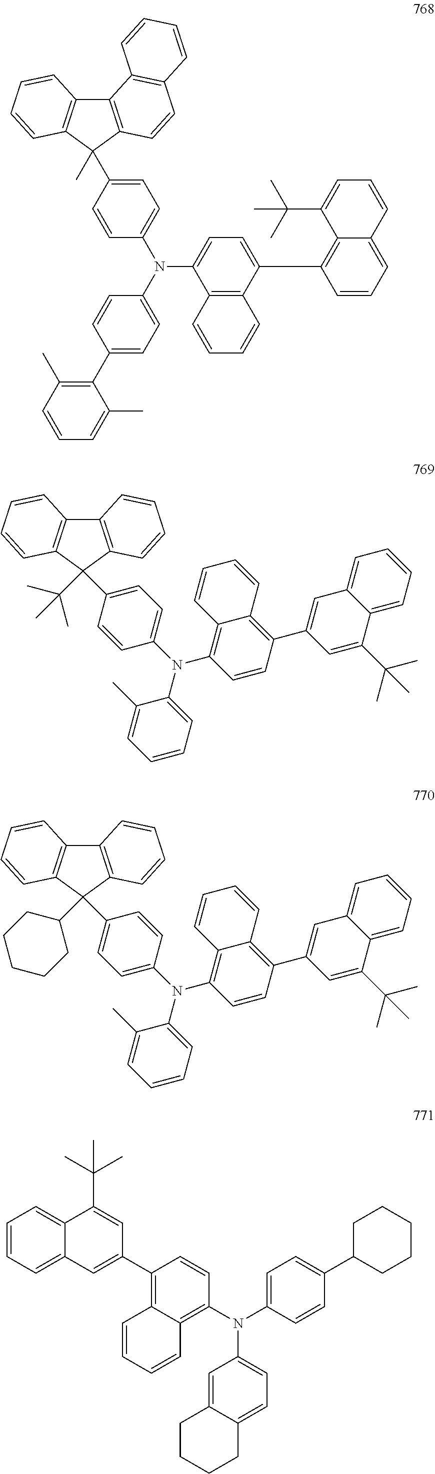 [SMILES: Cc1cccc(C)c1-c1ccc(N(c2ccc(C3(C)c4ccccc4-c4c3ccc3ccccc43)cc2)c2ccc(-c3cccc4cccc(C(C)(C)C)c34)c3ccccc23)cc1 ; Cc1ccccc1N(c1ccc(C2(C(C)(C)C)c3ccccc3-c3ccccc32)cc1)c1ccc(-c2cc(C(C)(C)C)c3ccccc3c2)c2ccccc12 ; Cc1ccccc1N(c1ccc(C2(C3CCCCC3)c3ccccc3-c3ccccc32)cc1)c1ccc(-c2cc(C(C)(C)C)c3ccccc3c2)c2ccccc12 ; CC(C)(C)c1cc(-c2ccc(N(c3ccc(C4CCCCC4)cc3)c3ccc4c(c3)CCCC4)c3ccccc23)cc2ccccc12]

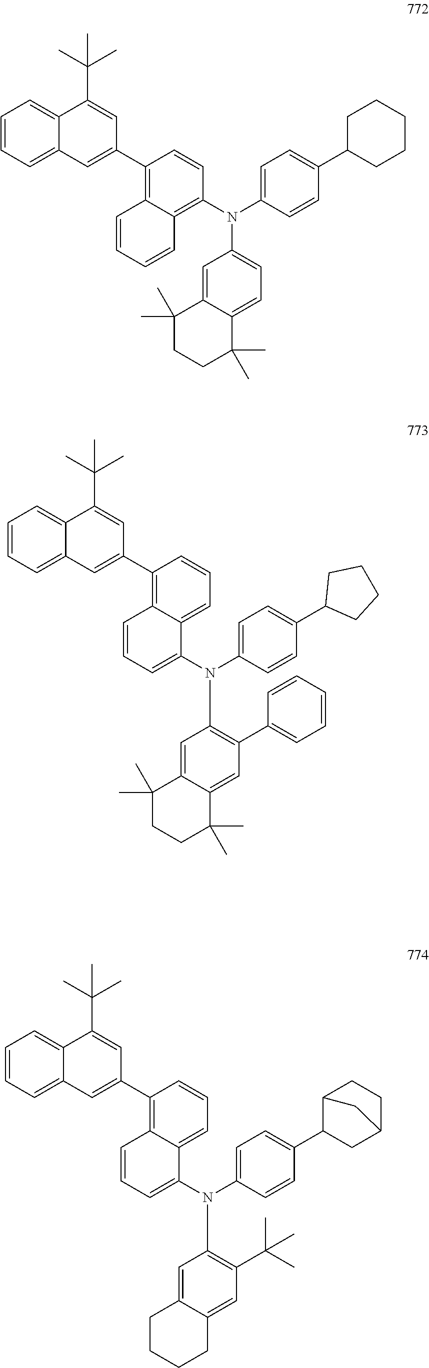 [SMILES: CC(C)(C)c1cc(-c2ccc(N(c3ccc(C4CCCCC4)cc3)c3ccc4c(c3)C(C)(C)CCC4(C)C)c3ccccc23)cc2ccccc12 ; CC(C)(C)c1cc(-c2cccc3c(N(c4ccc(C5CCCC5)cc4)c4cc5c(cc4-c4ccccc4)C(C)(C)CCC5(C)C)cccc23)cc2ccccc12 ; CC(C)(C)c1cc2c(cc1N(c1ccc(C3CC4CCC3C4)cc1)c1cccc3c(-c4cc(C(C)(C)C)c5ccccc5c4)cccc13)CCCC2]

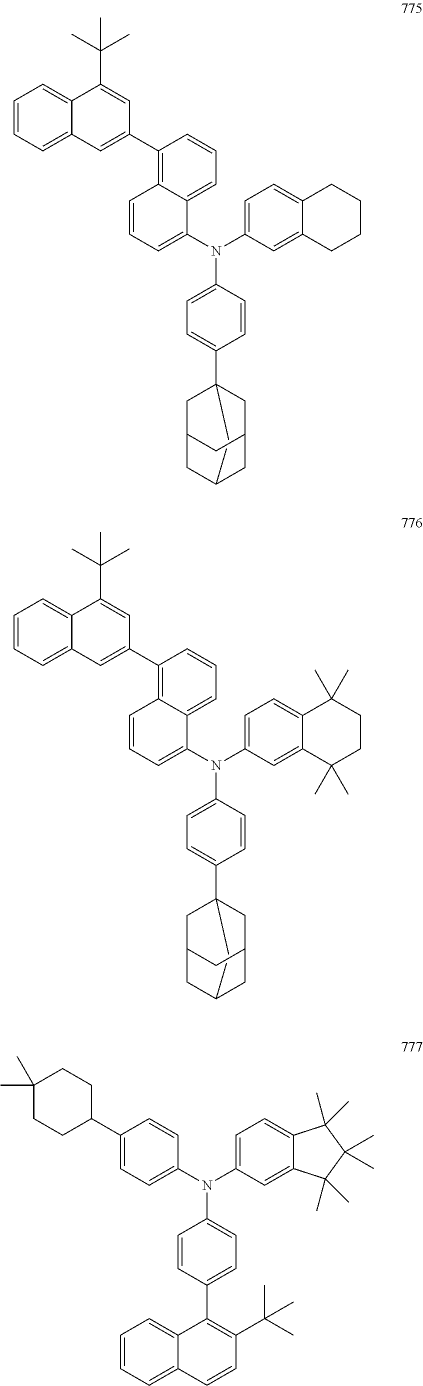

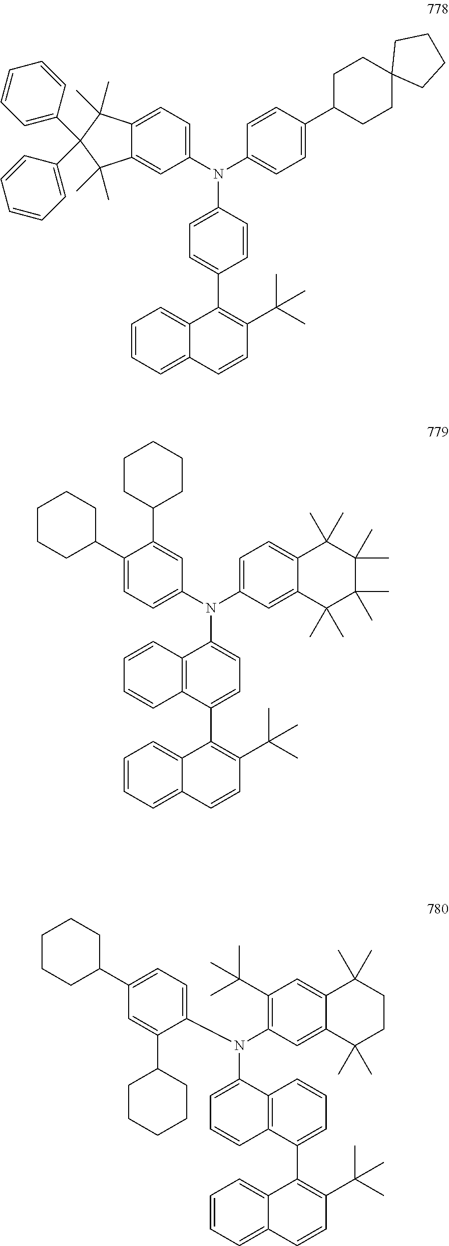 [SMILES: CC(C)(C)c1ccc2ccccc2c1-c1ccc(N(c2ccc(C3CCC4(CCCC4)CC3)cc2)c2ccc3c(c2)C(C)(C)C(c2ccccc2)(c2ccccc2)C3(C)C)cc1 ; CC(C)(C)c1ccc2ccccc2c1-c1ccc(N(c2ccc(C3CCCCC3)c(C3CCCCC3)c2)c2ccc3c(c2)C(C)(C)C(C)(C)C(C)(C)C3(C)C)c2ccccc12 ; CC(C)(C)c1cc2c(cc1N(c1ccc(C3CCCCC3)cc1C1CCCCC1)c1cccc3c(-c4c(C(C)(C)C)ccc5ccccc45)cccc13)C(C)(C)CCC2(C)C]

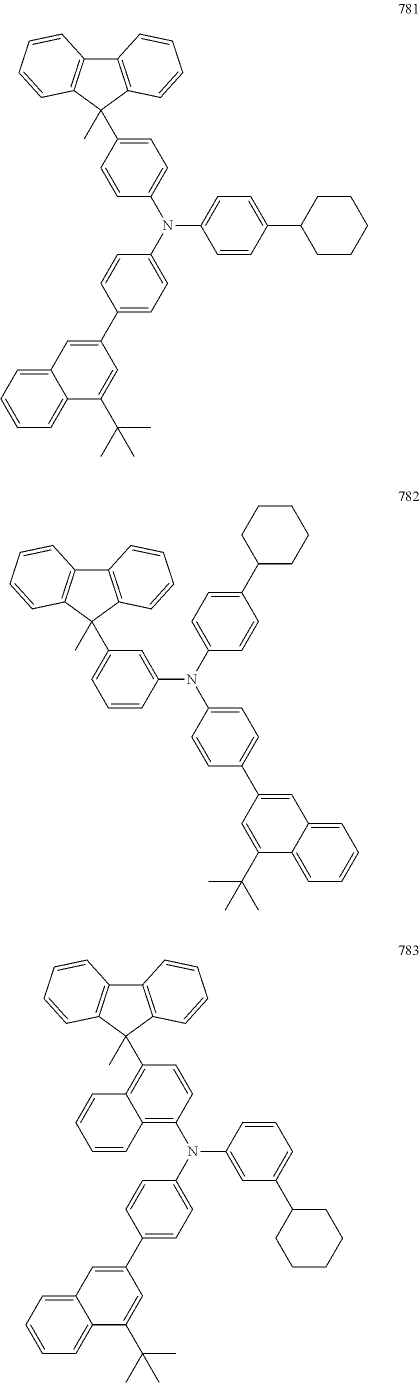 [SMILES: CC(C)(C)c1cc(-c2ccc(N(c3ccc(C4CCCCC4)cc3)c3ccc(C4(C)c5ccccc5-c5ccccc54)cc3)cc2)cc2ccccc12 ; CC(C)(C)c1cc(-c2ccc(N(c3ccc(C4CCCCC4)cc3)c3cccc(C4(C)c5ccccc5-c5ccccc54)c3)cc2)cc2ccccc12 ; CC(C)(C)c1cc(-c2ccc(N(c3cccc(C4CCCCC4)c3)c3ccc(C4(C)c5ccccc5-c5ccccc54)c4ccccc34)cc2)cc2ccccc12]

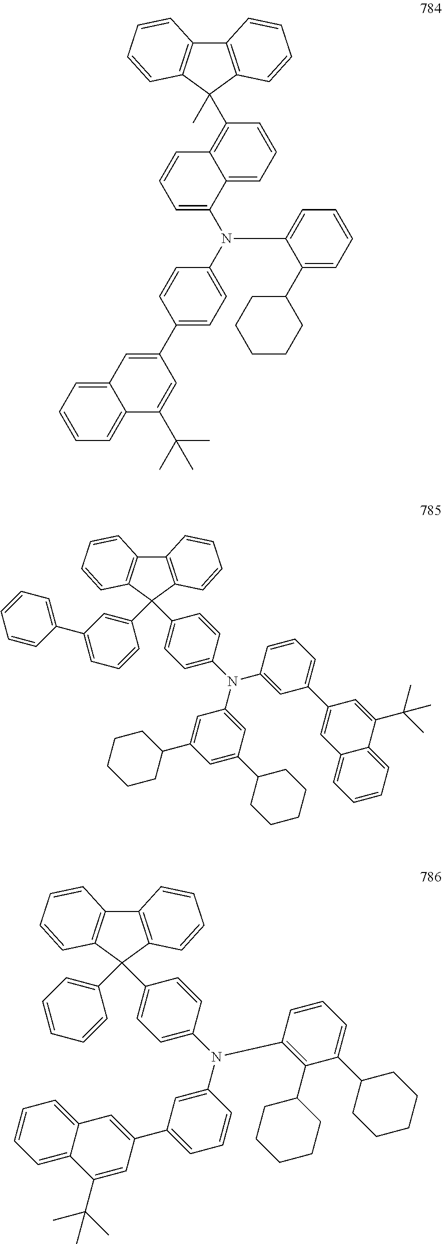 [SMILES: CC(C)(C)c1cc(-c2ccc(N(c3ccccc3C3CCCCC3)c3cccc4c(C5(C)c6ccccc6-c6ccccc65)cccc34)cc2)cc2ccccc12 ; CC(C)(C)c1cc(-c2cccc(N(c3ccc(C4(c5cccc(-c6ccccc6)c5)c5ccccc5-c5ccccc54)cc3)c3cc(C4CCCCC4)cc(C4CCCCC4)c3)c2)cc2ccccc12 ; CC(C)(C)c1cc(-c2cccc(N(c3ccc(C4(c5ccccc5)c5ccccc5-c5ccccc54)cc3)c3cccc(C4CCCCC4)c3C3CCCCC3)c2)cc2ccccc12]

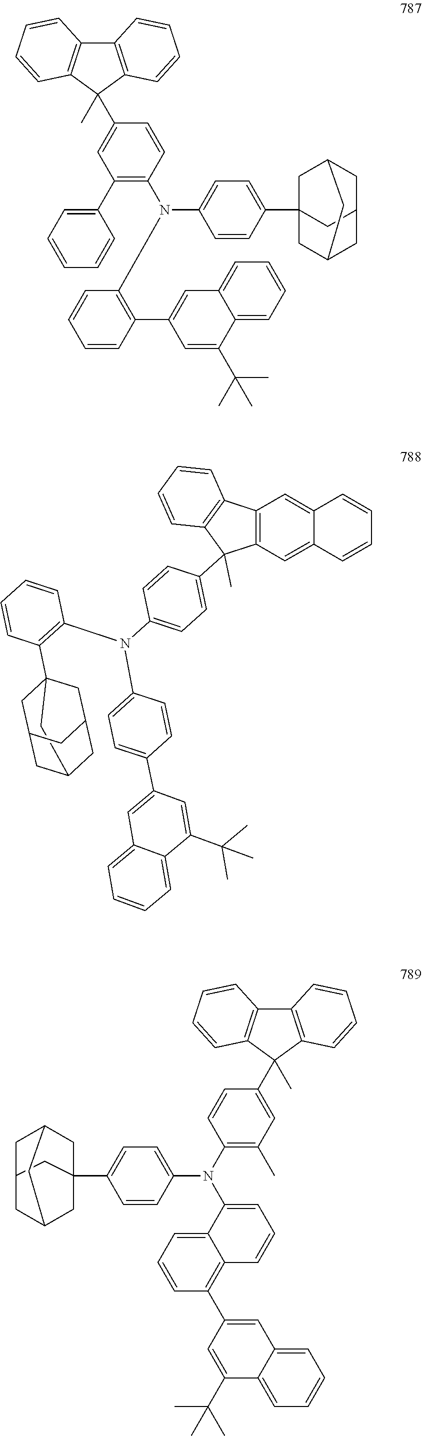 [SMILES: CC(C)(C)c1cc(-c2ccccc2N(c2ccc(C34CC5CC(CC(C5)C3)C4)cc2)c2ccc(C3(C)c4ccccc4-c4ccccc43)cc2-c2ccccc2)cc2ccccc12 ; CC(C)(C)c1cc(-c2ccc(N(c3ccc(C4(C)c5ccccc5-c5cc6ccccc6cc54)cc3)c3ccccc3C34CC5CC(CC(C5)C3)C4)cc2)cc2ccccc12 ; Cc1cc(C2(C)c3ccccc3-c3ccccc32)ccc1N(c1ccc(C23CC4CC(CC(C4)C2)C3)cc1)c1cccc2c(-c3cc(C(C)(C)C)c4ccccc4c3)cccc12]

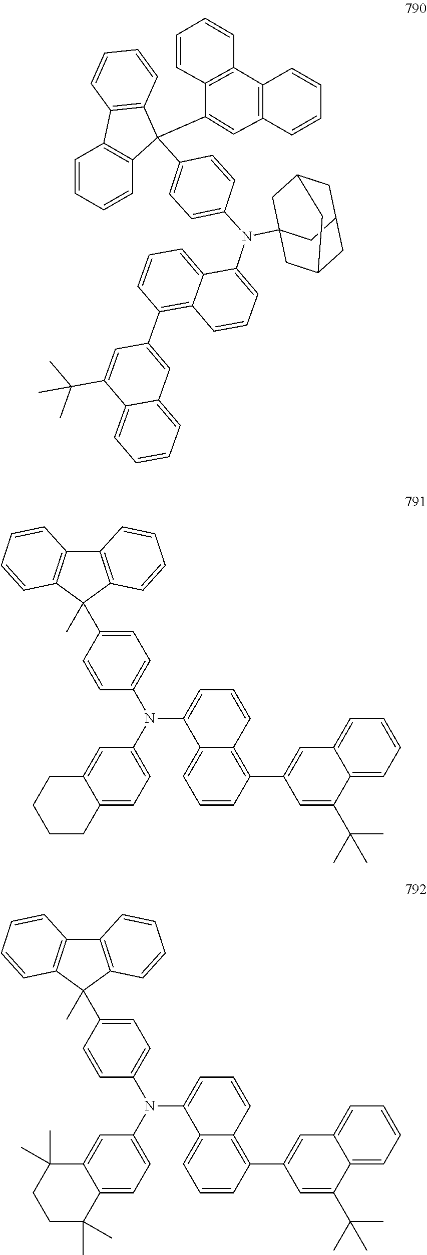 [SMILES: CC(C)(C)c1cc(-c2cccc3c(N(c4ccc(C5(c6cc7ccccc7c7ccccc67)c6ccccc6-c6ccccc65)cc4)C45CC6CC(CC(C6)C4)C5)cccc23)cc2ccccc12 ; CC(C)(C)c1cc(-c2cccc3c(N(c4ccc(C5(C)c6ccccc6-c6ccccc65)cc4)c4ccc5c(c4)CCCC5)cccc23)cc2ccccc12 ; CC(C)(C)c1cc(-c2cccc3c(N(c4ccc(C5(C)c6ccccc6-c6ccccc65)cc4)c4ccc5c(c4)C(C)(C)CCC5(C)C)cccc23)cc2ccccc12]

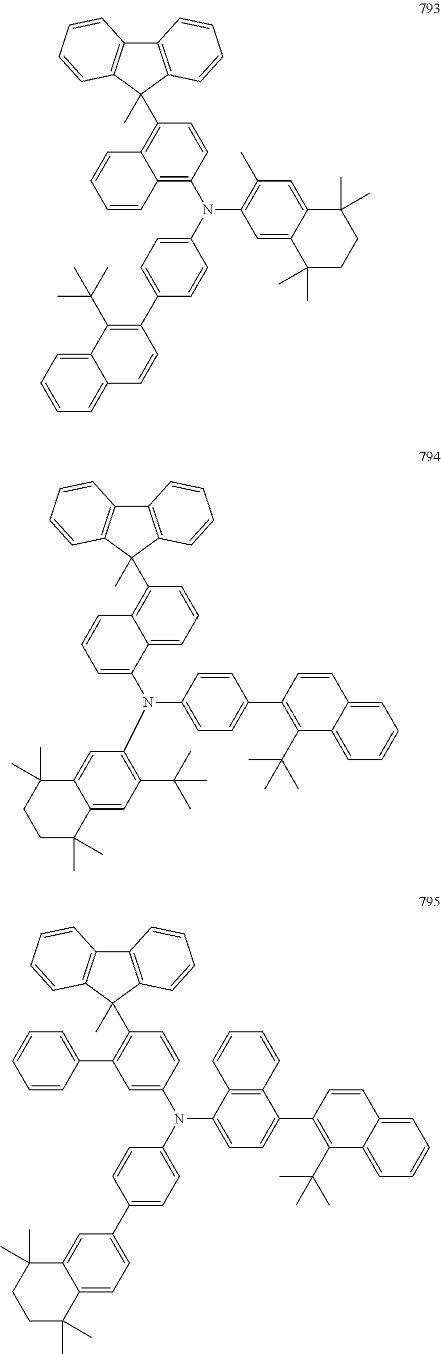

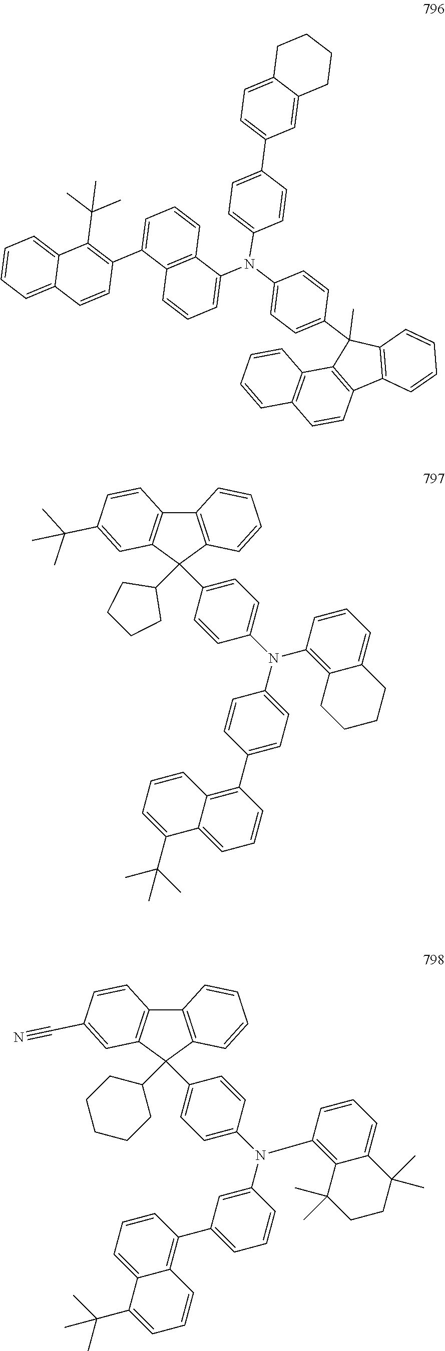 [SMILES: CC(C)(C)c1c(-c2cccc3c(N(c4ccc(-c5ccc6c(c5)CCCC6)cc4)c4ccc(C5(C)c6ccccc6-c6ccc7ccccc7c65)cc4)cccc23)ccc2ccccc12 ; CC(C)(C)c1ccc2c(c1)C(c1ccc(N(c3ccc(-c4cccc5c(C(C)(C)C)cccc45)cc3)c3cccc4c3CCCC4)cc1)(C1CCCC1)c1ccccc1-2 ; CC(C)(C)c1cccc2c(-c3cccc(N(c4ccc(C5(C6CCCCC6)c6ccccc6-c6ccc(C#N)cc65)cc4)c4cccc5c4C(C)(C)CCC5(C)C)c3)cccc12]

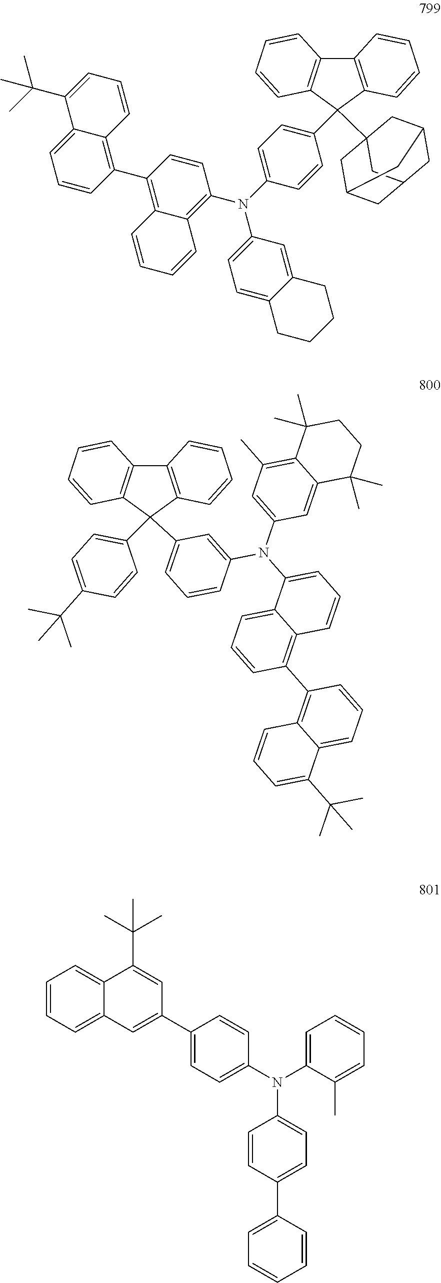 [SMILES: CC(C)(C)c1cccc2c(-c3ccc(N(c4ccc(C5(C67CC8CC(CC(C8)C6)C7)c6ccccc6-c6ccccc65)cc4)c4ccc5c(c4)CCCC5)c4ccccc34)cccc12 ; Cc1cc(N(c2cccc(C3(c4ccc(C(C)(C)C)cc4)c4ccccc4-c4ccccc43)c2)c2cccc3c(-c4cccc5c(C(C)(C)C)cccc45)cccc23)cc2c1C(C)(C)CCC2(C)C ; Cc1ccccc1N(c1ccc(-c2ccccc2)cc1)c1ccc(-c2cc(C(C)(C)C)c3ccccc3c2)cc1]

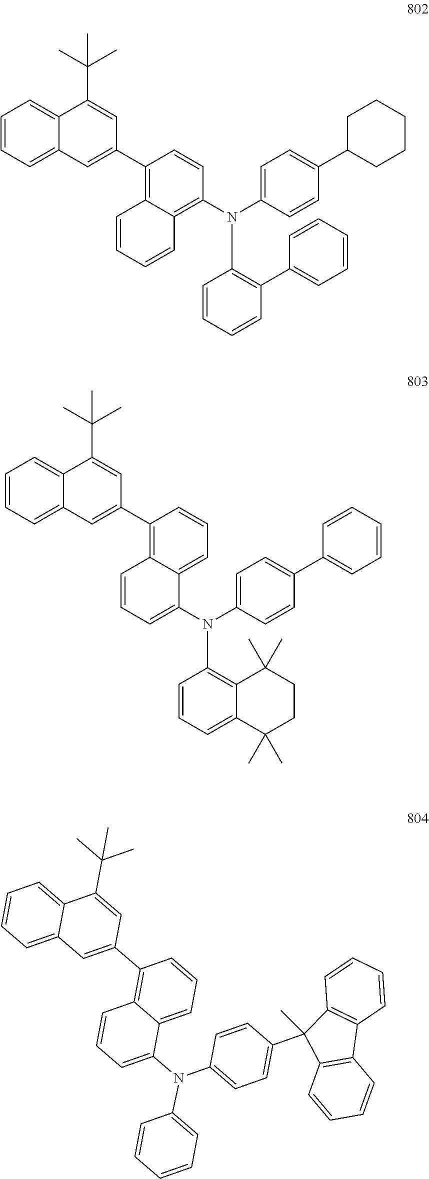 [SMILES: CC(C)(C)c1cc(-c2ccc(N(c3ccc(C4CCCCC4)cc3)c3ccccc3-c3ccccc3)c3ccccc23)cc2ccccc12 ; CC(C)(C)c1cc(-c2cccc3c(N(c4ccc(-c5ccccc5)cc4)c4cccc5c4C(C)(C)CCC5(C)C)cccc23)cc2ccccc12 ; CC(C)(C)c1cc(-c2cccc3c(N(c4ccccc4)c4ccc(C5(C)c6ccccc6-c6ccccc65)cc4)cccc23)cc2ccccc12]

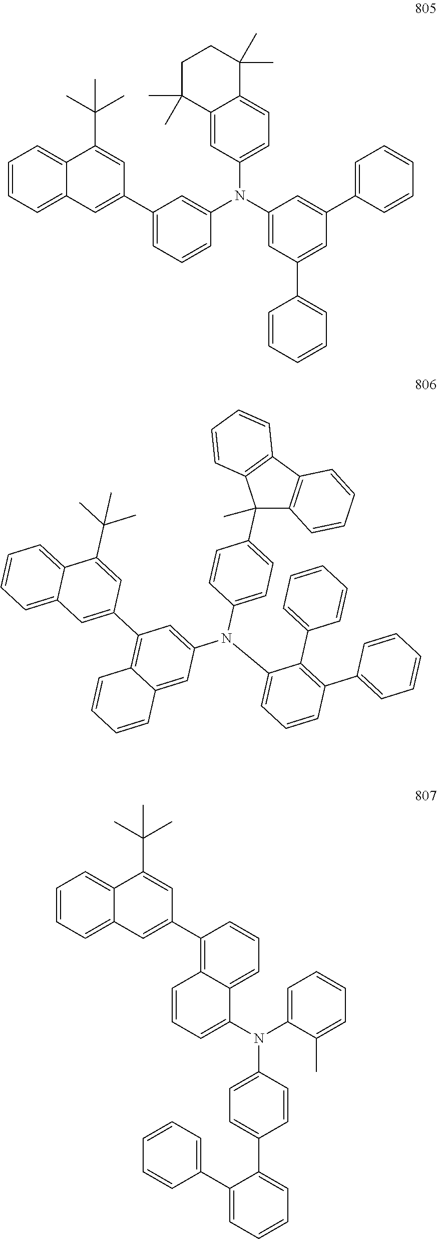 [SMILES: CC(C)(C)c1cc(-c2cccc(N(c3cc(-c4ccccc4)cc(-c4ccccc4)c3)c3ccc4c(c3)C(C)(C)CCC4(C)C)c2)cc2ccccc12 ; CC(C)(C)c1cc(-c2cc(N(c3ccc(C4(C)c5ccccc5-c5ccccc54)cc3)c3cccc(-c4ccccc4)c3-c3ccccc3)cc3ccccc23)cc2ccccc12 ; Cc1ccccc1N(c1ccc(-c2ccccc2-c2ccccc2)cc1)c1cccc2c(-c3cc(C(C)(C)C)c4ccccc4c3)cccc12]

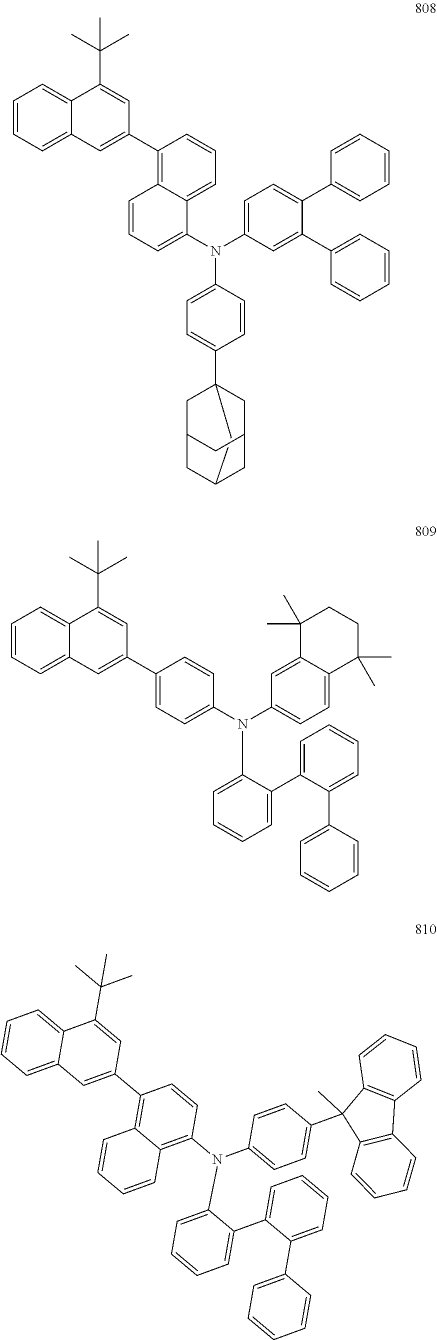 [SMILES: CC(C)(C)c1cc(-c2cccc3c(N(c4ccc(C56CC7CC(CC(C7)C5)C6)cc4)c4ccc(-c5ccccc5)c(-c5ccccc5)c4)cccc23)cc2ccccc12 ; CC(C)(C)c1cc(-c2ccc(N(c3ccc4c(c3)C(C)(C)CCC4(C)C)c3ccccc3-c3ccccc3-c3ccccc3)cc2)cc2ccccc12 ; CC(C)(C)c1cc(-c2ccc(N(c3ccc(C4(C)c5ccccc5-c5ccccc54)cc3)c3ccccc3-c3ccccc3-c3ccccc3)c3ccccc23)cc2ccccc12]

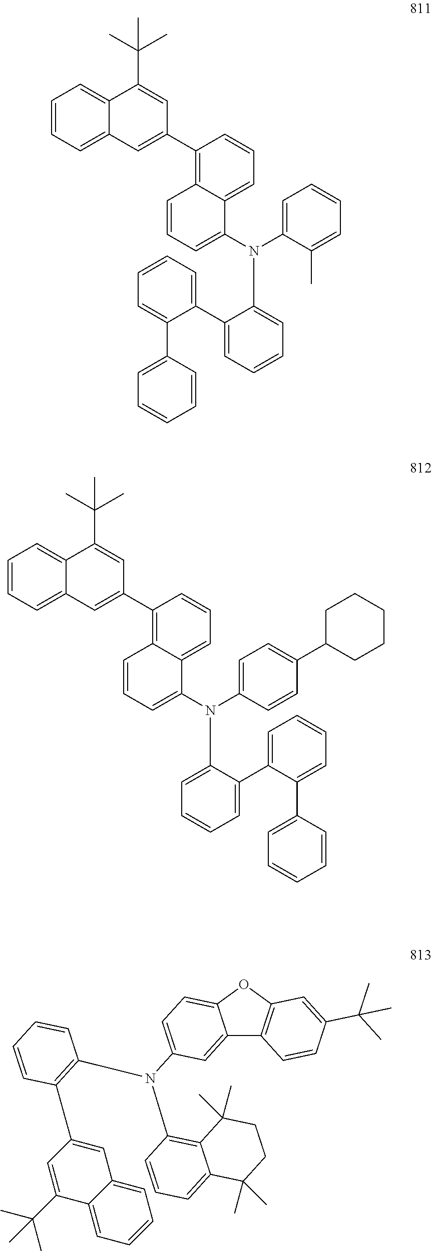 [SMILES: Cc1ccccc1N(c1ccccc1-c1ccccc1-c1ccccc1)c1cccc2c(-c3cc(C(C)(C)C)c4ccccc4c3)cccc12 ; CC(C)(C)c1cc(-c2cccc3c(N(c4ccc(C5CCCCC5)cc4)c4ccccc4-c4ccccc4-c4ccccc4)cccc23)cc2ccccc12 ; CC(C)(C)c1ccc2c(c1)oc1ccc(N(c3ccccc3-c3cc(C(C)(C)C)c4ccccc4c3)c3cccc4c3C(C)(C)CCC4(C)C)cc12]

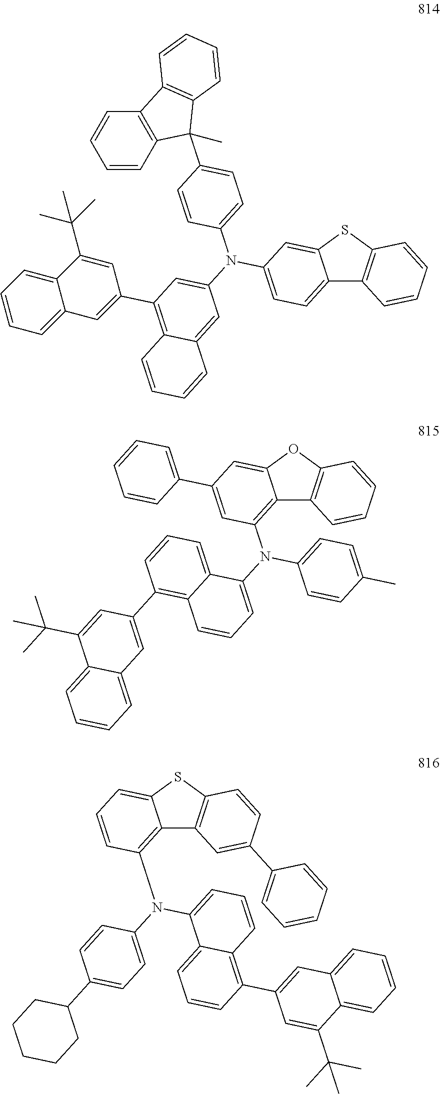 [SMILES: CC(C)(C)c1cc(-c2cc(N(c3ccc(C4(C)c5ccccc5-c5ccccc54)cc3)c3ccc4c(c3)sc3ccccc34)cc3ccccc23)cc2ccccc12 ; Cc1ccc(N(c2cccc3c(-c4cc(C(C)(C)C)c5ccccc5c4)cccc23)c2cc(-c3ccccc3)cc3oc4ccccc4c23)cc1 ; CC(C)(C)c1cc(-c2cccc3c(N(c4ccc(C5CCCCC5)cc4)c4cccc5sc6ccc(-c7ccccc7)cc6c45)cccc23)cc2ccccc12]

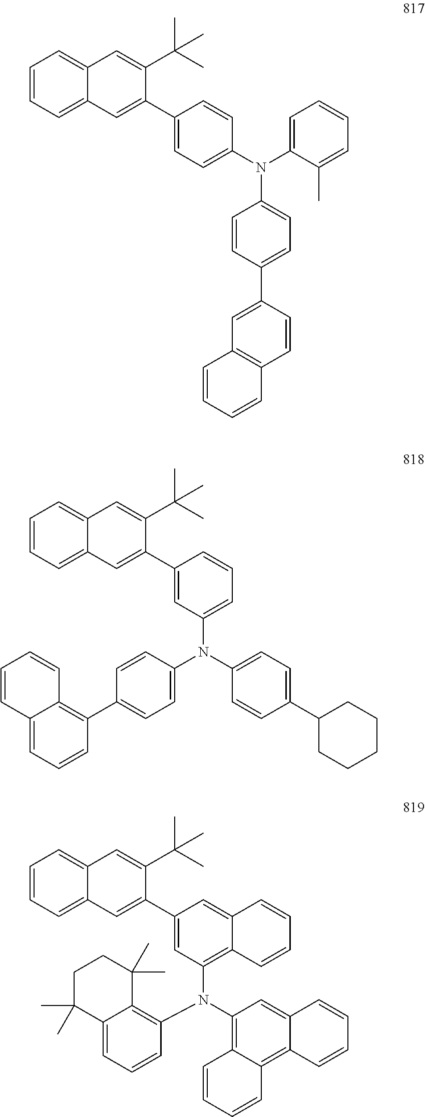 [SMILES: Cc1ccccc1N(c1ccc(-c2ccc3ccccc3c2)cc1)c1ccc(-c2cc3ccccc3cc2C(C)(C)C)cc1 ; CC(C)(C)c1cc2ccccc2cc1-c1cccc(N(c2ccc(-c3cccc4ccccc34)cc2)c2ccc(C3CCCCC3)cc2)c1 ; CC(C)(C)c1cc2ccccc2cc1-c1cc(N(c2cccc3c2C(C)(C)CCC3(C)C)c2cc3ccccc3c3ccccc23)c2ccccc2c1]

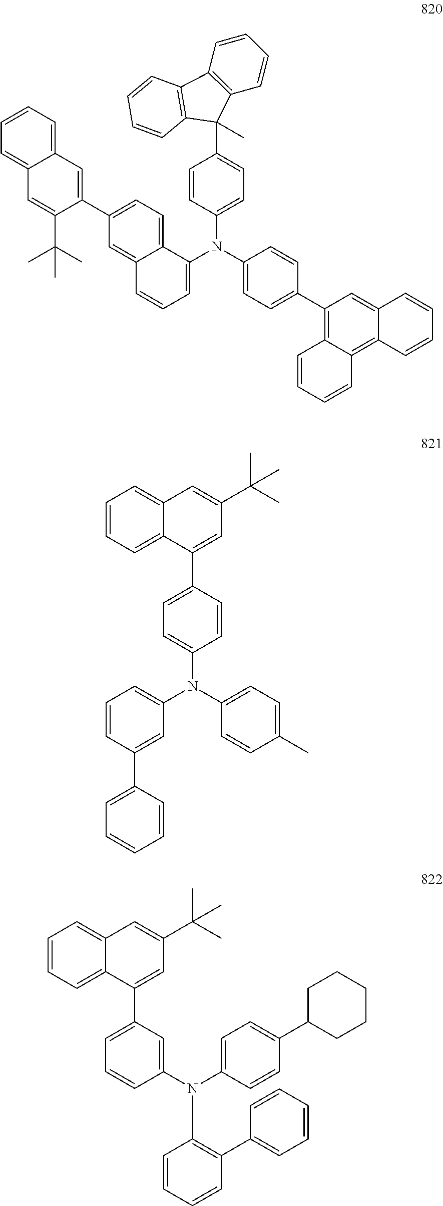 [SMILES: CC(C)(C)c1cc2ccccc2cc1-c1ccc2c(N(c3ccc(-c4cc5ccccc5c5ccccc45)cc3)c3ccc(C4(C)c5ccccc5-c5ccccc54)cc3)cccc2c1 ; Cc1ccc(N(c2ccc(-c3cc(C(C)(C)C)cc4ccccc34)cc2)c2cccc(-c3ccccc3)c2)cc1 ; CC(C)(C)c1cc(-c2cccc(N(c3ccc(C4CCCCC4)cc3)c3ccccc3-c3ccccc3)c2)c2ccccc2c1]

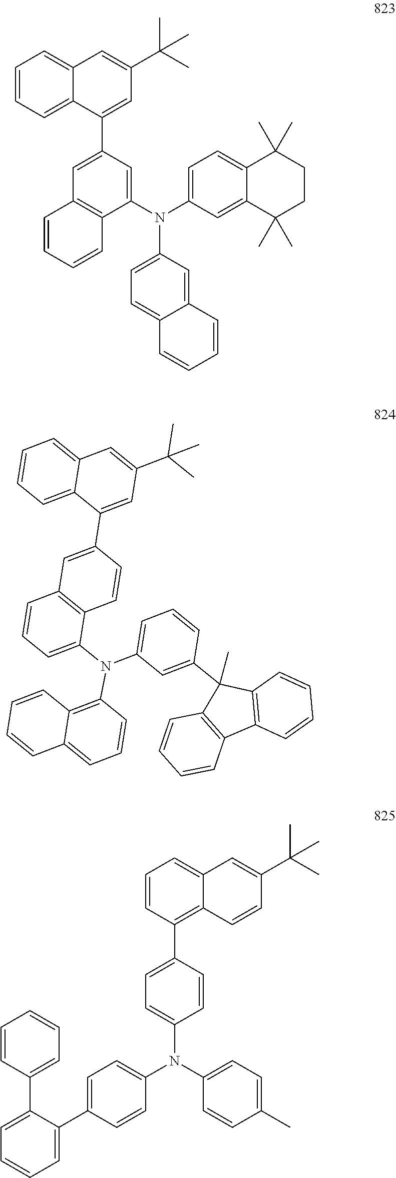 [SMILES: CC(C)(C)c1cc(-c2cc(N(c3ccc4c(c3)C(C)(C)CCC4(C)C)c3ccc4ccccc4c3)c3ccccc3c2)c2ccccc2c1 ; CC(C)(C)c1cc(-c2ccc3c(N(c4cccc(C5(C)c6ccccc6-c6ccccc65)c4)c4cccc5ccccc45)cccc3c2)c2ccccc2c1 ; Cc1ccc(N(c2ccc(-c3ccccc3-c3ccccc3)cc2)c2ccc(-c3cccc4cc(C(C)(C)C)ccc34)cc2)cc1]

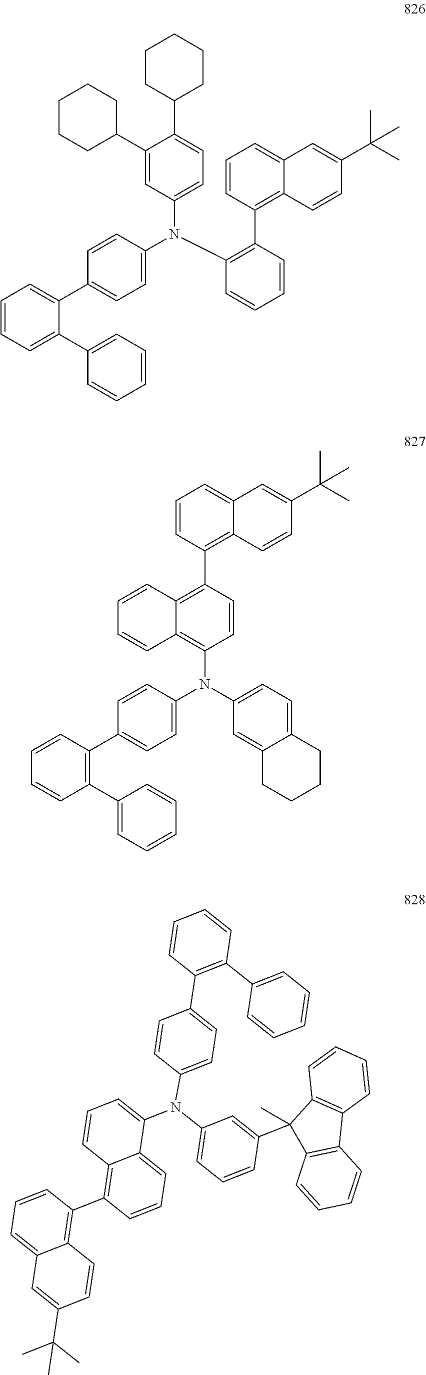 [SMILES: CC(C)(C)c1ccc2c(-c3ccccc3N(c3ccc(-c4ccccc4-c4ccccc4)cc3)c3ccc(C4CCCCC4)c(C4CCCCC4)c3)cccc2c1 ; CC(C)(C)c1ccc2c(-c3ccc(N(c4ccc(-c5ccccc5-c5ccccc5)cc4)c4ccc5c(c4)CCCC5)c4ccccc34)cccc2c1 ; CC(C)(C)c1ccc2c(-c3cccc4c(N(c5ccc(-c6ccccc6-c6ccccc6)cc5)c5cccc(C6(C)c7ccccc7-c7ccccc76)c5)cccc34)cccc2c1]

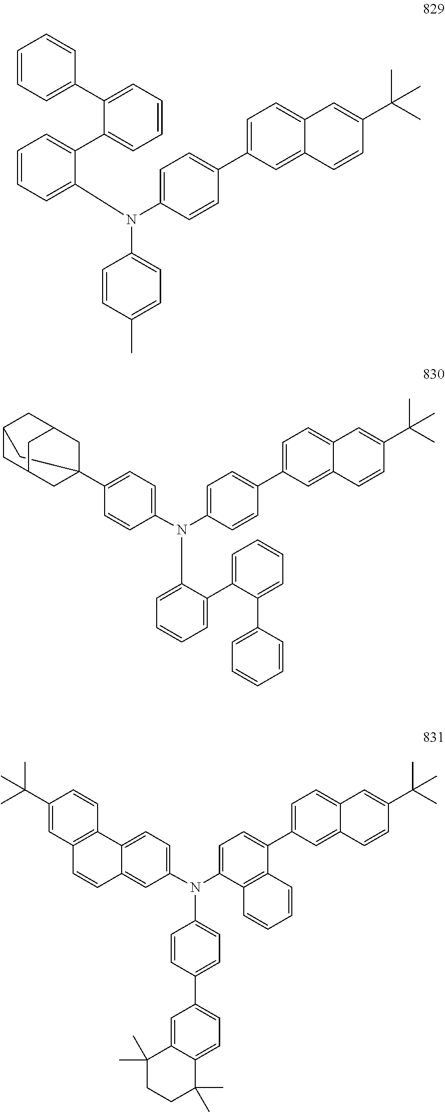

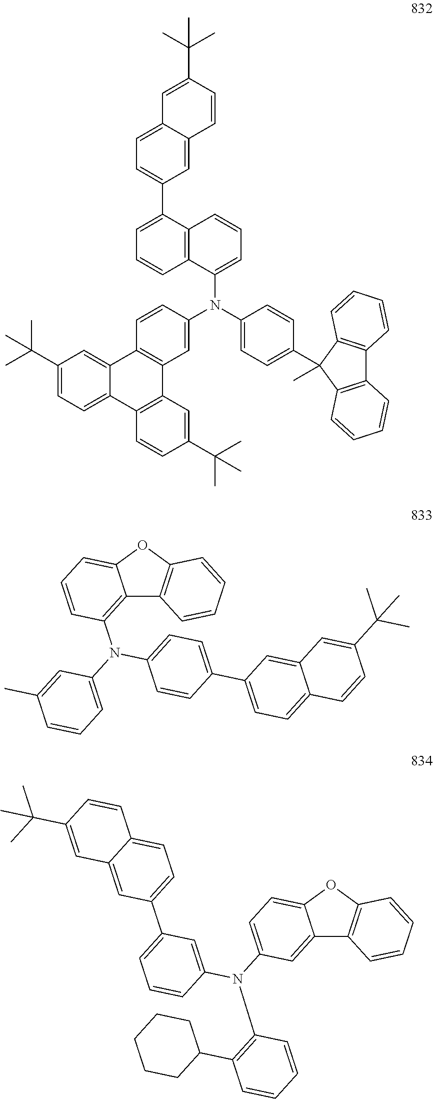

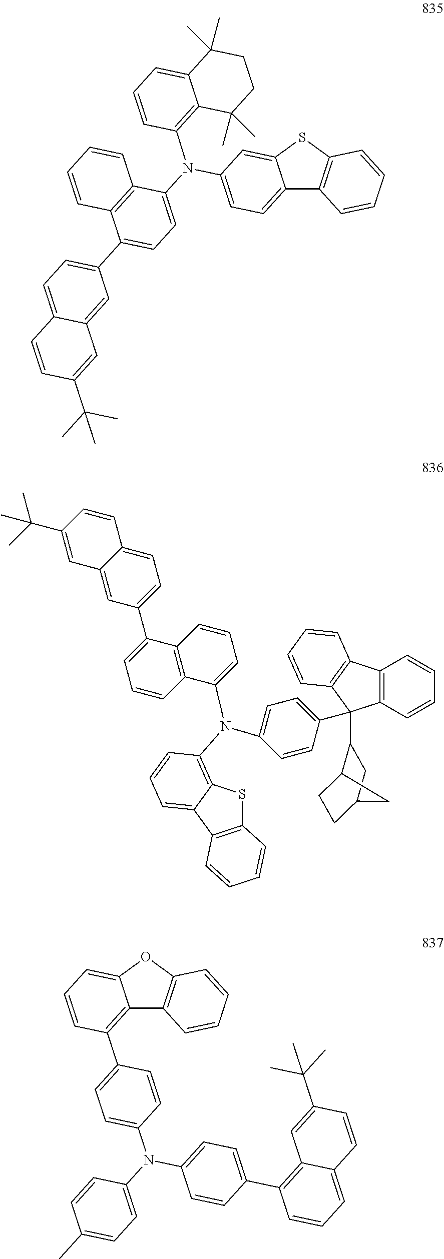 [SMILES: CC(C)(C)c1ccc2ccc(-c3ccc(N(c4ccc5c(c4)sc4ccccc45)c4cccc5c4C(C)(C)CCC5(C)C)c4ccccc34)cc2c1 ; CC(C)(C)c1ccc2ccc(-c3cccc4c(N(c5ccc(C6(C7CC8CCC7C8)c7ccccc7-c7ccccc76)cc5)c5cccc6c5sc5ccccc56)cccc34)cc2c1 ; Cc1ccc(N(c2ccc(-c3cccc4ccc(C(C)(C)C)cc34)cc2)c2ccc(-c3cccc4oc5ccccc5c34)cc2)cc1]

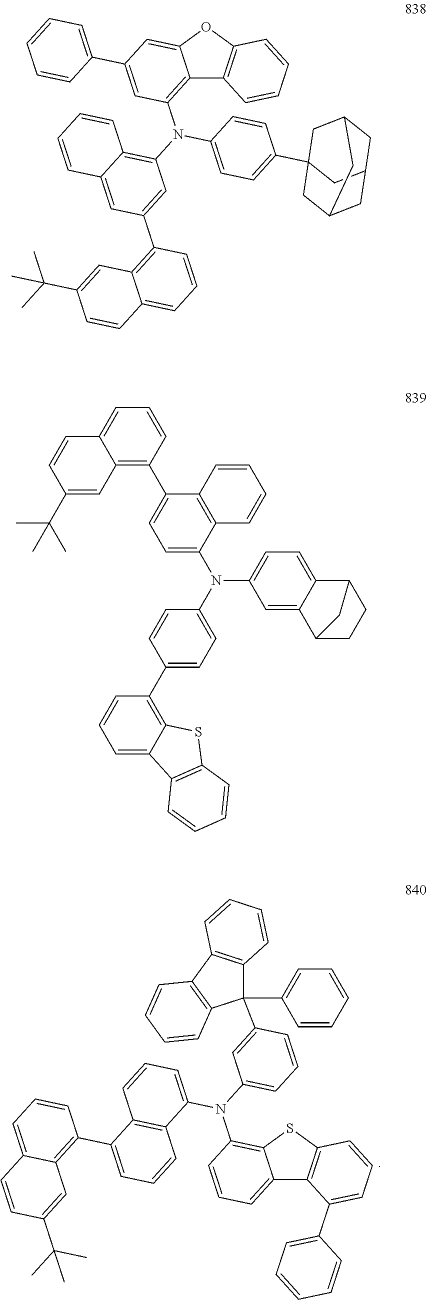 [SMILES: CC(C)(C)c1ccc2cccc(-c3cc(N(c4ccc(C56CC7CC(CC(C7)C5)C6)cc4)c4cc(-c5ccccc5)cc5oc6ccccc6c45)c4ccccc4c3)c2c1 ; CC(C)(C)c1ccc2cccc(-c3ccc(N(c4ccc(-c5cccc6c5sc5ccccc56)cc4)c4ccc5c(c4)C4CCC5C4)c4ccccc34)c2c1 ; CC(C)(C)c1ccc2cccc(-c3cccc4c(N(c5cccc(C6(c7ccccc7)c7ccccc7-c7ccccc76)c5)c5cccc6c5sc5cccc(-c7ccccc7)c56)cccc34)c2c1]

8. An organic light-emitting device comprising a first electrode; a second electrode facing the first electrode; and one or more organic layer provided between the first electrode and the second electrode, wherein one or more layer of the organic layer comprises the compound according to claim 1.

9. The organic light-emitting device according to claim 8, wherein the organic layer comprises one or more layer selected from a group consisting of a light-emitting layer, a hole injection layer, a hole transport layer, a hole transport auxiliary layer, an electron blocking layer, a hole blocking layer, an electron transport layer and an electron injection layer.

10. The organic light-emitting device according to claim 8, wherein the organic layer comprises a hole transport layer, and the hole transport layer comprises the compound.

11. The organic light-emitting device according to claim 8, wherein the organic layer comprises an electron blocking layer, and the electron blocking layer comprises the compound.
